SEQUENCE LISTING (842 sequences)

SEQ ID NO: 1 - RNA, 22 nt, Homo sapiens
  source 1..22: mol_type = transcribed RNA
ggggctgtgattgaccagcagg

SEQ ID NO: 2 - RNA, 23 nt, Homo sapiens
  source 1..23: mol_type = transcribed RNA
ccccggtgttggggcgcgtctgc

SEQ ID NO: 3 - RNA, 21 nt, Homo sapiens
  source 1..21: mol_type = transcribed RNA
gtgggcgggggcaggtgtgtg

SEQ ID NO: 4 - RNA, 17 nt, Homo sapiens
  source 1..17: mol_type = transcribed RNA
ccccggggagcccggcg

SEQ ID NO: 5 - RNA, 20 nt, Homo sapiens
  source 1..20: mol_type = transcribed RNA
ctaggtggggggcttgaagc

SEQ ID NO: 6 - RNA, 20 nt, Homo sapiens
  source 1..20: mol_type = transcribed RNA
gccggggctttgggtgaggg

SEQ ID NO: 7 - RNA, 20 nt, Homo sapiens
  source 1..20: mol_type = transcribed RNA
gtgggttggggcgggctctg

SEQ ID NO: 8 - RNA, 22 nt, Homo sapiens
  source 1..22: mol_type = transcribed RNA
actcggcgtggcgtcggtcgtg

SEQ ID NO: 9 - RNA, 22 nt, Homo sapiens
  source 1..22: mol_type = transcribed RNA
acctggcagcagggagcgtcgt

SEQ ID NO: 10 - RNA, 24 nt, Homo sapiens
  source 1..24: mol_type = transcribed RNA
tgaggggcctcagaccgagctttt

SEQ ID NO: 11 - RNA, 22 nt, Homo sapiens
  source 1..22: mol_type = transcribed RNA
agtgggaggccagggcacggca

SEQ ID NO: 12 - RNA, 20 nt, Homo sapiens
  source 1..20: mol_type = transcribed RNA
agagatgaagcgggggggcg

SEQ ID NO: 13 - RNA, 21 nt, Homo sapiens
  source 1..21: mol_type = transcribed RNA
atccagttctctgagggggct

SEQ ID NO: 14 - RNA, 22 nt, Homo sapiens
  source 1..22: mol_type = transcribed RNA
aaaagctgggttgagagggcaa

SEQ ID NO: 15 - RNA, 24 nt, Homo sapiens
  source 1..24: mol_type = transcribed RNA
tgggcgaggggtgggctctcagag

SEQ ID NO: 16 - RNA, 21 nt, Homo sapiens
  source 1..21: mol_type = transcribed RNA
gtaggtgacagtcaggggcgg

SEQ ID NO: 17 - RNA, 22 nt, Homo sapiens
  source 1..22: mol_type = transcribed RNA
ctcctggggcccgcactctcgc

SEQ ID NO: 18 - RNA, 23 nt, Homo sapiens
  source 1..23: mol_type = transcribed RNA
ctggcggagcccattccatgcca

SEQ ID NO: 19 - RNA, 25 nt, Homo sapiens
  source 1..25: mol_type = transcribed RNA
aggggggcactgcgcaagcaaagcc

SEQ ID NO: 20 - RNA, 22 nt, Homo sapiens
  source 1..22: mol_type = transcribed RNA
ttcagatcccagcggtgcctct

SEQ ID NO: 21 - RNA, 21 nt, Homo sapiens
  source 1..21: mol_type = transcribed RNA
cccggagccaggatgcagctc

SEQ ID NO: 22 - RNA, 23 nt, Homo sapiens
  source 1..23: mol_type = transcribed RNA
agggtggggctggaggtggggct

SEQ ID NO: 23 - RNA, 22 nt, Homo sapiens
  source 1..22: mol_type = transcribed RNA
actcaaaatgggggcgctttcc

SEQ ID NO: 24 - RNA, 18 nt, Homo sapiens
  source 1..18: mol_type = transcribed RNA
cgggcgtggtggtggggg

SEQ ID NO: 25 - RNA, 19 nt, Homo sapiens
  source 1..19: mol_type = transcribed RNA
atcccaccactgccaccat

SEQ ID NO: 26 - RNA, 17 nt, Homo sapiens
  source 1..17: mol_type = transcribed RNA
ccccgccaccgccttgg

SEQ ID NO: 27 - RNA, 22 nt, Homo sapiens
  source 1..22: mol_type = transcribed RNA
agggggcgcagtcactgacgtg

SEQ ID NO: 28 - RNA, 22 nt, Homo sapiens
  source 1..22: mol_type = transcribed RNA
ctcggcgcggggcgcgggctcc

SEQ ID NO: 29 - RNA, 22 nt, Homo sapiens
  source 1..22: mol_type = transcribed RNA
aggactggactcccggcagccc

SEQ ID NO: 30 - RNA, 22 nt, Homo sapiens
  source 1..22: mol_type = transcribed RNA
actgggtaggtggggctccagg

SEQ ID NO: 31 - RNA, 23 nt, Homo sapiens
  source 1..23: mol_type = transcribed RNA
gtgcgtggtggctcgaggcgggg

SEQ ID NO: 32 - RNA, 19 nt, Homo sapiens
  source 1..19: mol_type = transcribed RNA
gagccagttggacaggagc

SEQ ID NO: 33 - RNA, 22 nt, Homo sapiens
  source 1..22: mol_type = transcribed RNA
tagggggcggcttgtggagtgt

SEQ ID NO: 34 - RNA, 23 nt, Homo sapiens
  source 1..23: mol_type = transcribed RNA
gtgagtgggagccggtggggctg

SEQ ID NO: 35 - RNA, 23 nt, Homo sapiens
  source 1..23: mol_type = transcribed RNA
tgagcaccacacaggccgggcgc

SEQ ID NO: 36 - RNA, 18 nt, Homo sapiens
  source 1..18: mol_type = transcribed RNA
cccagcaggacgggagcg

SEQ ID NO: 37 - RNA, 23 nt, Homo sapiens
  source 1..23: mol_type = transcribed RNA
ccagggggatgggcgagcttggg

SEQ ID NO: 38 - RNA, 21 nt, Homo sapiens
  source 1..21: mol_type = transcribed RNA
cgggccggaggtcaagggcgt

SEQ ID NO: 39 - RNA, 22 nt, Homo sapiens
  source 1..22: mol_type = transcribed RNA
aggcagcggggtgtagtggata

SEQ ID NO: 40 - RNA, 21 nt, Homo sapiens
  source 1..21: mol_type = transcribed RNA
accactgcactccagcctgag

SEQ ID NO: 41 - RNA, 24 nt, Homo sapiens
  source 1..24: mol_type = transcribed RNA
gatgcgccgcccactgccccgcgc

SEQ ID NO: 42 - RNA, 20 nt, Homo sapiens
  source 1..20: mol_type = transcribed RNA
ggatccgagtcacggcacca

SEQ ID NO: 43 - RNA, 25 nt, Homo sapiens
  source 1..25: mol_type = transcribed RNA
agcggggaggaagtgggcgctgctt

SEQ ID NO: 44 - RNA, 22 nt, Homo sapiens
  source 1..22: mol_type = transcribed RNA
acgcccttcccccccttcttca

SEQ ID NO: 45 - RNA, 22 nt, Homo sapiens
  source 1..22: mol_type = transcribed RNA
gtgaacgggcgccatcccgagg

SEQ ID NO: 46 - RNA, 21 nt, Homo sapiens
  source 1..21: mol_type = transcribed RNA
gcggtggggccggaggggcgt

SEQ ID NO: 47 - RNA, 23 nt, Homo sapiens
  source 1..23: mol_type = transcribed RNA
gtgagtgggagccccagtgtgtg

SEQ ID NO: 48 - RNA, 22 nt, Homo sapiens
  source 1..22: mol_type = transcribed RNA
tcgggcctggggttgggggagc

SEQ ID NO: 49 - RNA, 22 nt, Homo sapiens
  source 1..22: mol_type = transcribed RNA
tgggcgagggcggctgagcggc

SEQ ID NO: 50 - RNA, 22 nt, Homo sapiens
  source 1..22: mol_type = transcribed RNA
tggggaggtgtggagtcagcat

SEQ ID NO: 51 - RNA, 22 nt, Homo sapiens
  source 1..22: mol_type = transcribed RNA
ggtggcccggccgtgcctgagg

SEQ ID NO: 52 - RNA, 22 nt, Homo sapiens
  source 1..22: mol_type = transcribed RNA
gtgaggaggggctggcagggac

SEQ ID NO: 53 - RNA, 22 nt, Homo sapiens
  source 1..22: mol_type = transcribed RNA
gagcaggcgaggctgggctgaa

SEQ ID NO: 54 - RNA, 25 nt, Homo sapiens
  source 1..25: mol_type = transcribed RNA
gtgaggcggggccaggagggtgtgt

SEQ ID NO: 55 - RNA, 18 nt, Homo sapiens
  source 1..18: mol_type = transcribed RNA
gggtgagggcaggtggtt

SEQ ID NO: 56 - RNA, 25 nt, Homo sapiens
  source 1..25: mol_type = transcribed RNA
tcggggcatgggggagggaggctgg

SEQ ID NO: 57 - RNA, 20 nt, Homo sapiens
  source 1..20: mol_type = transcribed RNA
actcaaactgtgggggcact

SEQ ID NO: 58 - RNA, 21 nt, Homo sapiens
  source 1..21: mol_type = transcribed RNA
tggggcggggcaggtccctgc

SEQ ID NO: 59 - RNA, 22 nt, Homo sapiens
  source 1..22: mol_type = transcribed RNA
gagggttgggtggaggctctcc

SEQ ID NO: 60 - RNA, 18 nt, Homo sapiens
  source 1..18: mol_type = transcribed RNA
ttcccagccaacgcacca

SEQ ID NO: 61 - RNA, 23 nt, Homo sapiens
  source 1..23: mol_type = transcribed RNA
ggatggttgggggcggtcggcgt

SEQ ID NO: 62 - RNA, 20 nt, Homo sapiens
  source 1..20: mol_type = transcribed RNA
tctcttcatctaccccccag

SEQ ID NO: 63 - RNA, 22 nt, Homo sapiens
  source 1..22: mol_type = transcribed RNA
cagccctcctcccgcacccaaa

SEQ ID NO: 64 - RNA, 22 nt, Homo sapiens
  source 1..22: mol_type = transcribed RNA
tggggagcggcccccgggtggg

SEQ ID NO: 65 - RNA, 21 nt, Homo sapiens
  source 1..21: mol_type = transcribed RNA
tgggggtgtggggagagagag

SEQ ID NO: 66 - RNA, 21 nt, Homo sapiens
  source 1..21: mol_type = transcribed RNA
ggggggatgtgcatgctggtt

SEQ ID NO: 67 - RNA, 21 nt, Homo sapiens
  source 1..21: mol_type = transcribed RNA
cctgcagagaggaagcccttc

SEQ ID NO: 68 - RNA, 22 nt, Homo sapiens
  source 1..22: mol_type = transcribed RNA
tggcgggggtagagctggctgc

SEQ ID NO: 69 - RNA, 23 nt, Homo sapiens
  source 1..23: mol_type = transcribed RNA
gaacgcctgttcttgccaggtgg

SEQ ID NO: 70 - RNA, 22 nt, Homo sapiens
  source 1..22: mol_type = transcribed RNA
ttgaggagacatggtgggggcc

SEQ ID NO: 71 - RNA, 22 nt, Homo sapiens
  source 1..22: mol_type = transcribed RNA
atatacagggggagactctcat

SEQ ID NO: 72 - RNA, 20 nt, Homo sapiens
  source 1..20: mol_type = transcribed RNA
cgggcgtggtggtgggggtg

SEQ ID NO: 73 - RNA, 20 nt, Homo sapiens
  source 1..20: mol_type = transcribed RNA
tcacacctgcctcgcccccc

SEQ ID NO: 74 - RNA, 22 nt, Homo sapiens
  source 1..22: mol_type = transcribed RNA
atatacagggggagactcttat

SEQ ID NO: 75 - RNA, 21 nt, Homo sapiens
  source 1..21: mol_type = transcribed RNA
aaggcagggcccccgctcccc

SEQ ID NO: 76 - RNA, 24 nt, Homo sapiens
  source 1..24: mol_type = transcribed RNA
tggggagctgaggctctgggggtg

SEQ ID NO: 77 - RNA, 22 nt, Homo sapiens
  source 1..22: mol_type = transcribed RNA
tagggatgggaggccaggatga

SEQ ID NO: 78 - RNA, 17 nt, Homo sapiens
  source 1..17: mol_type = transcribed RNA
gtgggggagaggctgtc

SEQ ID NO: 79 - RNA, 24 nt, Homo sapiens
  source 1..24: mol_type = transcribed RNA
agggctggactcagcggcggagct

SEQ ID NO: 80 - RNA, 23 nt, Homo sapiens
  source 1..23: mol_type = transcribed RNA
tcaataggaaagaggtgggacct

SEQ ID NO: 81 - RNA, 21 nt, Homo sapiens
  source 1..21: mol_type = transcribed RNA
tcacctggctggcccgcccag

SEQ ID NO: 82 - RNA, 22 nt, Homo sapiens
  source 1..22: mol_type = transcribed RNA
cttccccccagtaatcttcatc

SEQ ID NO: 83 - RNA, 21 nt, Homo sapiens
  source 1..21: mol_type = transcribed RNA
agctgtacctgaaaccaagca

SEQ ID NO: 84 - RNA, 17 nt, Homo sapiens
  source 1..17: mol_type = transcribed RNA
accccactcctggtacc

SEQ ID NO: 85 - RNA, 22 nt, Homo sapiens
  source 1..22: mol_type = transcribed RNA
ggggaactgtagatgaaaaggc

SEQ ID NO: 86 - RNA, 17 nt, Homo sapiens
  source 1..17: mol_type = transcribed RNA
ggtgggggctgttgttt

SEQ ID NO: 87 - RNA, 20 nt, Homo sapiens
  source 1..20: mol_type = transcribed RNA
ggctccttggtctaggggta

SEQ ID NO: 88 - RNA, 25 nt, Homo sapiens
  source 1..25: mol_type = transcribed RNA
ggcggagggaagtaggtccgttggt

SEQ ID NO: 89 - RNA, 22 nt, Homo sapiens
  source 1..22: mol_type = transcribed RNA
tgattgtcttcccccaccctca

SEQ ID NO: 90 - RNA, 23 nt, Homo sapiens
  source 1..23: mol_type = transcribed RNA
cgggtagagagggcagtgggagg

SEQ ID NO: 91 - RNA, 23 nt, Homo sapiens
  source 1..23: mol_type = transcribed RNA
tggggggacagatggagaggaca

SEQ ID NO: 92 - RNA, 22 nt, Homo sapiens
  source 1..22: mol_type = transcribed RNA
agtggggtgggacccagctgtt

SEQ ID NO: 93 - RNA, 21 nt, Homo sapiens
  source 1..21: mol_type = transcribed RNA
tcatccccctcgccctctcag

SEQ ID NO: 94 - RNA, 23 nt, Homo sapiens
  source 1..23: mol_type = transcribed RNA
ccggggcagattggtgtagggtg

SEQ ID NO: 95 - RNA, 18 nt, Homo sapiens
  source 1..18: mol_type = transcribed RNA
atcctagtcacggcacca

SEQ ID NO: 96 - RNA, 18 nt, Homo sapiens
  source 1..18: mol_type = transcribed RNA
aggctgggctgggacgga

SEQ ID NO: 97 - RNA, 23 nt, Homo sapiens
  source 1..23: mol_type = transcribed RNA
tcggggagtctggggtccggaat

SEQ ID NO: 98 - RNA, 22 nt, Homo sapiens
  source 1..22: mol_type = transcribed RNA
agcaaggcggcatctctctgat

SEQ ID NO: 99 - RNA, 19 nt, Homo sapiens
  source 1..19: mol_type = transcribed RNA
ggctggtcagatgggagtg

SEQ ID NO: 100 - RNA, 22 nt, Homo sapiens
  source 1..22: mol_type = transcribed RNA
tgagcccctgtgccgcccccag

SEQ ID NO: 101 - RNA, 19 nt, Homo sapiens
  source 1..19: mol_type = transcribed RNA
agcagggctggggattgca

SEQ ID NO: 102 - RNA, 22 nt, Homo sapiens
  source 1..22: mol_type = transcribed RNA
gctgcgggctgcggtcagggcg

SEQ ID NO: 103 - RNA, 22 nt, Homo sapiens
  source 1..22: mol_type = transcribed RNA
agctctgctgctcactggcagt

SEQ ID NO: 104 - RNA, 25 nt, Homo sapiens
  source 1..25: mol_type = transcribed RNA
agggatcgcgggcgggtggcggcct

SEQ ID NO: 105 - RNA, 22 nt, Homo sapiens
  source 1..22: mol_type = transcribed RNA
agcagaggcagagaggctcagg

SEQ ID NO: 106 - RNA, 23 nt, Homo sapiens
  source 1..23: mol_type = transcribed RNA
tgggaggggagaggcagcaagca

SEQ ID NO: 107 - RNA, 21 nt, Homo sapiens
  source 1..21: mol_type = transcribed RNA
gttggggtgcaggggtctgct

SEQ ID NO: 108 - RNA, 24 nt, Homo sapiens
  source 1..24: mol_type = transcribed RNA
gtaggggcgtcccgggcgcgcggg

SEQ ID NO: 109 - RNA, 22 nt, Homo sapiens
  source 1..22: mol_type = transcribed RNA
tcaaaatcaggagtcggggctt

SEQ ID NO: 110 - RNA, 20 nt, Homo sapiens
  source 1..20: mol_type = transcribed RNA
aaaagctgggctgagaggcg

SEQ ID NO: 111 - RNA, 22 nt, Homo sapiens
  source 1..22: mol_type = transcribed RNA
gcccaggactttgtgcggggtg

SEQ ID NO: 112 - RNA, 22 nt, Homo sapiens
  source 1..22: mol_type = transcribed RNA
caggaaggatttagggacaggc

SEQ ID NO: 113 - RNA, 23 nt, Homo sapiens
  source 1..23: mol_type = transcribed RNA
tggtgcggagagggcccacagtg

SEQ ID NO: 114 - RNA, 22 nt, Homo sapiens
  source 1..22: mol_type = transcribed RNA
gggacccagggagagacgtaag

SEQ ID NO: 115 - RNA, 21 nt, Homo sapiens
  source 1..21: mol_type = transcribed RNA
tgagggacccaggacaggaga

SEQ ID NO: 116 - RNA, 22 nt, Homo sapiens
  source 1..22: mol_type = transcribed RNA
ggctttctagtctcagctctcc

SEQ ID NO: 117 - RNA, 21 nt, Homo sapiens
  source 1..21: mol_type = transcribed RNA
cggcggggacggcgattggtc

SEQ ID NO: 118 - RNA, 21 nt, Homo sapiens
  source 1..21: mol_type = transcribed RNA
cgggagctggggtctgcaggt

SEQ ID NO: 119 - RNA, 22 nt, Homo sapiens
  source 1..22: mol_type = transcribed RNA
tctgccccctccgctgctgcca

SEQ ID NO: 120 - RNA, 20 nt, Homo sapiens
  source 1..20: mol_type = transcribed RNA
cggtggactggagtgggtgg

SEQ ID NO: 121 - RNA, 21 nt, Homo sapiens
  source 1..21: mol_type = transcribed RNA
agccgcggggatcgccgaggg

SEQ ID NO: 122 - RNA, 23 nt, Homo sapiens
  source 1..23: mol_type = transcribed RNA
tgtagagcagggagcaggaagct

SEQ ID NO: 123 - RNA, 22 nt, Homo sapiens
  source 1..22: mol_type = transcribed RNA
gcgggggtggcggcggcatccc

SEQ ID NO: 124 - RNA, 20 nt, Homo sapiens
  source 1..20: mol_type = transcribed RNA
gctcggactgagcaggtggg

SEQ ID NO: 125 - RNA, 22 nt, Homo sapiens
  source 1..22: mol_type = transcribed RNA
gctgggattacaggcatgagcc

SEQ ID NO: 126 - RNA, 20 nt, Homo sapiens
  source 1..20: mol_type = transcribed RNA
gtgtctgggcggacagctgc

SEQ ID NO: 127 - RNA, 21 nt, Homo sapiens
  source 1..21: mol_type = transcribed RNA
aggggtgctatctgtgattga

SEQ ID NO: 128 - RNA, 21 nt, Homo sapiens
  source 1..21: mol_type = transcribed RNA
cgtcccaccccccactcctgt

SEQ ID NO: 129 - RNA, 24 nt, Homo sapiens
  source 1..24: mol_type = transcribed RNA
cgggtgggagcagatcttattgag

SEQ ID NO: 130 - RNA, 23 nt, Homo sapiens
  source 1..23: mol_type = transcribed RNA
gccccggcgcgggcgggttctgg

SEQ ID NO: 131 - RNA, 21 nt, Homo sapiens
  source 1..21: mol_type = transcribed RNA
tctgtggagtggggtgcctgt

SEQ ID NO: 132 - RNA, 21 nt, Homo sapiens
  source 1..21: mol_type = transcribed RNA
cccatgcctcctgccgcggtc

SEQ ID NO: 133 - RNA, 23 nt, Homo sapiens
  source 1..23: mol_type = transcribed RNA
tggggaaggcttggcagggaaga

SEQ ID NO: 134 - RNA, 21 nt, Homo sapiens
  source 1..21: mol_type = transcribed RNA
aagggacagggagggtcgtgg

SEQ ID NO: 135 - RNA, 21 nt, Homo sapiens
  source 1..21: mol_type = transcribed RNA
ctaccccccatccccctgtag

SEQ ID NO: 136 - RNA, 20 nt, Homo sapiens
  source 1..20: mol_type = transcribed RNA
accaggaggctgaggcccct

SEQ ID NO: 137 - RNA, 23 nt, Homo sapiens
  source 1..23: mol_type = transcribed RNA
tgggggctgggatgggccatggt

SEQ ID NO: 138 - RNA, 22 nt, Homo sapiens
  source 1..22: mol_type = transcribed RNA
tgaggcccttggggcacagtgg

SEQ ID NO: 139 - RNA, 17 nt, Homo sapiens
  source 1..17: mol_type = transcribed RNA
gggagtctacagcaggg

SEQ ID NO: 140 - RNA, 23 nt, Homo sapiens
  source 1..23: mol_type = transcribed RNA
caggcagaagtggggctgacagg

SEQ ID NO: 141 - RNA, 22 nt, Homo sapiens
  source 1..22: mol_type = transcribed RNA
atggccagagctcacacagagg

SEQ ID NO: 142 - RNA, 22 nt, Homo sapiens
  source 1..22: mol_type = transcribed RNA
aaggcccgggctttcctcccag

SEQ ID NO: 143 - RNA, 21 nt, Homo sapiens
  source 1..21: mol_type = transcribed RNA
ccgccttctctcctcccccag

SEQ ID NO: 144 - RNA, 22 nt, Homo sapiens
  source 1..22: mol_type = transcribed RNA
gtgagtagtggcgcgcggcggc

SEQ ID NO: 145 - RNA, 18 nt, Homo sapiens
  source 1..18: mol_type = transcribed RNA
ctggcagggggagaggta

SEQ ID NO: 146 - RNA, 18 nt, Homo sapiens
  source 1..18: mol_type = transcribed RNA
atcccacctctgccacca

SEQ ID NO: 147 - RNA, 22 nt, Homo sapiens
  source 1..22: mol_type = transcribed RNA
agggccgaagggtggaagctgc

SEQ ID NO: 148 - RNA, 23 nt, Homo sapiens
  source 1..23: mol_type = transcribed RNA
tgggcaggggcttattgtaggag

SEQ ID NO: 149 - RNA, 23 nt, Homo sapiens
  source 1..23: mol_type = transcribed RNA
tgggctgagggcaggaggcctgt

SEQ ID NO: 150 - RNA, 24 nt, Homo sapiens
  source 1..24: mol_type = transcribed RNA
gtgggtagggtttgggggagagcg

SEQ ID NO: 151 - RNA, 21 nt, Homo sapiens
  source 1..21: mol_type = transcribed RNA
acaggagtgggggtgggacat

SEQ ID NO: 152 - RNA, 18 nt, Homo sapiens
  source 1..18: mol_type = transcribed RNA
cagcagtccctccccctg

SEQ ID NO: 153 - RNA, 18 nt, Homo sapiens
  source 1..18: mol_type = transcribed RNA
gaggctgaaggaagatgg

SEQ ID NO: 154 - RNA, 21 nt, Homo sapiens
  source 1..21: mol_type = transcribed RNA
ctgggctcgggacgcgcggct

SEQ ID NO: 155 - RNA, 21 nt, Homo sapiens
  source 1..21: mol_type = transcribed RNA
caggcaggtgtagggtggagc

SEQ ID NO: 156 - RNA, 21 nt, Homo sapiens
  source 1..21: mol_type = transcribed RNA
ctccccggcctctgcccccag

SEQ ID NO: 157 - RNA, 23 nt, Homo sapiens
  source 1..23: mol_type = transcribed RNA
cggggccatggagcagcctgtgt

SEQ ID NO: 158 - RNA, 23 nt, Homo sapiens
  source 1..23: mol_type = transcribed RNA
cgaggggtagaagagcacagggg

SEQ ID NO: 159 - RNA, 22 nt, Homo sapiens
  source 1..22: mol_type = transcribed RNA
agactgacggctggaggcccat

SEQ ID NO: 160 - RNA, 22 nt, Homo sapiens
  source 1..22: mol_type = transcribed RNA
ccgggagaaggaggtggcctgg

SEQ ID NO: 161 - RNA, 22 nt, Homo sapiens
  source 1..22: mol_type = transcribed RNA
tggtggaggaagagggcagctc

SEQ ID NO: 162 - RNA, 19 nt, Homo sapiens
  source 1..19: mol_type = transcribed RNA
aggcaggttatctgggctg

SEQ ID NO: 163 - RNA, 21 nt, Homo sapiens
  source 1..21: mol_type = transcribed RNA
cttccgccccgccgggcgtcg

SEQ ID NO: 164 - RNA, 22 nt, Homo sapiens
  source 1..22: mol_type = transcribed RNA
aggcgatgtggggatgtagaga

SEQ ID NO: 165 - RNA, 21 nt, Homo sapiens
  source 1..21: mol_type = transcribed RNA
cgtggaggacgaggaggaggc

SEQ ID NO: 166 - RNA, 20 nt, Homo sapiens
  source 1..20: mol_type = transcribed RNA
cggctctgggtctgtgggga

SEQ ID NO: 167 - RNA, 20 nt, Homo sapiens
  source 1..20: mol_type = transcribed RNA
cctgagcccgggccgcgcag

SEQ ID NO: 168 - RNA, 23 nt, Homo sapiens
  source 1..23: mol_type = transcribed RNA
caggggctggggtttcaggttct

SEQ ID NO: 169 - RNA, 21 nt, Homo sapiens
  source 1..21: mol_type = transcribed RNA
aggtgggtatggaggagccct

SEQ ID NO: 170 - RNA, 23 nt, Homo sapiens
  source 1..23: mol_type = transcribed RNA
gggatggtagaccggtgacgtgc

SEQ ID NO: 171 - RNA, 20 nt, Homo sapiens
  source 1..20: mol_type = transcribed RNA
acccgcccgtctccccacag

SEQ ID NO: 172 - RNA, 22 nt, Homo sapiens
  source 1..22: mol_type = transcribed RNA
gtgggtgctggtgggagccgtg

SEQ ID NO: 173 - RNA, 22 nt, Homo sapiens
  source 1..22: mol_type = transcribed RNA
ctgggacaggaggaggaggcag

SEQ ID NO: 174 - RNA, 21 nt, Homo sapiens
  source 1..21: mol_type = transcribed RNA
gaagctctcccctccccgcag

SEQ ID NO: 175 - RNA, 22 nt, Homo sapiens
  source 1..22: mol_type = transcribed RNA
ctcgggcggaggtggttgagtg

SEQ ID NO: 176 - RNA, 22 nt, Homo sapiens
  source 1..22: mol_type = transcribed RNA
tgggagggcgtggatgatggtg

SEQ ID NO: 177 - RNA, 20 nt, Homo sapiens
  source 1..20: mol_type = transcribed RNA
tcgcgccccggctcccgttc

SEQ ID NO: 178 - RNA, 20 nt, Homo sapiens
  source 1..20: mol_type = transcribed RNA
cgcccctcctgcccccacag

SEQ ID NO: 179 - RNA, 21 nt, Homo sapiens
  source 1..21: mol_type = transcribed RNA
cacctctcctggcatcgcccc

SEQ ID NO: 180 - RNA, 23 nt, Homo sapiens
  source 1..23: mol_type = transcribed RNA
ggcaggagggctgtgccaggttg

SEQ ID NO: 181 - RNA, 21 nt, Homo sapiens
  source 1..21: mol_type = transcribed RNA
agcggtgctcctgcgggccga

SEQ ID NO: 182 - RNA, 22 nt, Homo sapiens
  source 1..22: mol_type = transcribed RNA
tggggatttggagaagtggtga

SEQ ID NO: 183 - RNA, 24 nt, Homo sapiens
  source 1..24: mol_type = transcribed RNA
tggggggacaggatgagaggctgt

SEQ ID NO: 184 - RNA, 23 nt, Homo sapiens
  source 1..23: mol_type = transcribed RNA
agggacttttgggggcagatgtg

SEQ ID NO: 185 - RNA, 23 nt, Homo sapiens
  source 1..23: mol_type = transcribed RNA
tttcaagccagggggcgtttttc

SEQ ID NO: 186 - RNA, 25 nt, Homo sapiens
  source 1..25: mol_type = transcribed RNA
aggagggaaggggctgagaacagga

SEQ ID NO: 187 - RNA, 21 nt, Homo sapiens
  source 1..21: mol_type = transcribed RNA
gccctccgcccgtgcaccccg

SEQ ID NO: 188 - RNA, 23 nt, Homo sapiens
  source 1..23: mol_type = transcribed RNA
aggaggtggtactaggggccagc

SEQ ID NO: 189 - RNA, 24 nt, Homo sapiens
  source 1..24: mol_type = transcribed RNA
ccccgggaacgtcgagactggagc

SEQ ID NO: 190 - RNA, 22 nt, Homo sapiens
  source 1..22: mol_type = transcribed RNA
agggaaggggacgagggttggg

SEQ ID NO: 191 - RNA, 22 nt, Homo sapiens
  source 1..22: mol_type = transcribed RNA
gagcttttggcccgggttatac

SEQ ID NO: 192 - RNA, 22 nt, Homo sapiens
  source 1..22: mol_type = transcribed RNA
tgggggtggtctctagccaagg

SEQ ID NO: 193 - RNA, 22 nt, Homo sapiens
  source 1..22: mol_type = transcribed RNA
ctggtacaggcctgggggacag

SEQ ID NO: 194 - RNA, 17 nt, Homo sapiens
  source 1..17: mol_type = transcribed RNA
ggatggaggaggggtct

SEQ ID NO: 195 - RNA, 22 nt, Homo sapiens
  source 1..22: mol_type = transcribed RNA
ggctggagcgagtgcagtggtg

SEQ ID NO: 196 - RNA, 23 nt, Homo sapiens
  source 1..23: mol_type = transcribed RNA
tcgaggactggtggaagggcctt

SEQ ID NO: 197 - RNA, 18 nt, Homo sapiens
  source 1..18: mol_type = transcribed RNA
cggtgagcgctcgctggc

SEQ ID NO: 198 - RNA, 22 nt, Homo sapiens
  source 1..22: mol_type = transcribed RNA
cagcaggggagagagaggagtc

SEQ ID NO: 199 - RNA, 21 nt, Homo sapiens
  source 1..21: mol_type = transcribed RNA
gggagtgcagggcagggtttc

SEQ ID NO: 200 - RNA, 22 nt, Homo sapiens
  source 1..22: mol_type = transcribed RNA
gggaccatcctgcctgctgtgg

SEQ ID NO: 201 - RNA, 23 nt, Homo sapiens
  source 1..23: mol_type = transcribed RNA
aggaagccctggaggggctggag

SEQ ID NO: 202 - RNA, 22 nt, Homo sapiens
  source 1..22: mol_type = transcribed RNA
actggggagcagaaggagaacc

SEQ ID NO: 203 - RNA, 18 nt, Homo sapiens
  source 1..18: mol_type = transcribed RNA
aggctggagtgagcggag

SEQ ID NO: 204 - RNA, 17 nt, Homo sapiens
  source 1..17: mol_type = transcribed RNA
cgcgccgggcccgggtt

SEQ ID NO: 205 - RNA, 23 nt, Homo sapiens
  source 1..23: mol_type = transcribed RNA
tgaggatatggcagggaagggga

SEQ ID NO: 206 - RNA, 21 nt, Homo sapiens
  source 1..21: mol_type = transcribed RNA
agcatgacagaggagaggtgg

SEQ ID NO: 207 - RNA, 21 nt, Homo sapiens
  source 1..21: mol_type = transcribed RNA
ttggagggtgtggaagacatc

SEQ ID NO: 208 - RNA, 19 nt, Homo sapiens
  source 1..19: mol_type = transcribed RNA
tgcggcagagctggggtca

SEQ ID NO: 209 - RNA, 19 nt, Homo sapiens
  source 1..19: mol_type = transcribed RNA
cggcgcgaccggcccgggg

SEQ ID NO: 210 - RNA, 22 nt, Homo sapiens
  source 1..22: mol_type = transcribed RNA
ggctacaacacaggacccgggc

SEQ ID NO: 211 - RNA, 19 nt, Homo sapiens
  source 1..19: mol_type = transcribed RNA
ctggggagtggctggggag

SEQ ID NO: 212 - RNA, 21 nt, Homo sapiens
  source 1..21: mol_type = transcribed RNA
ccggccgccggctccgccccg

SEQ ID NO: 213 - RNA, 21 nt, Homo sapiens
  source 1..21: mol_type = transcribed RNA
ccgtcgccgccacccgagccg

SEQ ID NO: 214 - RNA, 25 nt, Homo sapiens
  source 1..25: mol_type = transcribed RNA
taggggtgggggaattcaggggtgt

SEQ ID NO: 215 - RNA, 22 nt, Homo sapiens
  source 1..22: mol_type = transcribed RNA
agggggatggcagagcaaaatt

SEQ ID NO: 216 - RNA, 19 nt, Homo sapiens
  source 1..19: mol_type = transcribed RNA
tgggggagatgggggttga

SEQ ID NO: 217 - RNA, 20 nt, Homo sapiens
  source 1..20: mol_type = transcribed RNA
gggaaaaggaagggggagga

SEQ ID NO: 218 - RNA, 24 nt, Homo sapiens
  source 1..24: mol_type = transcribed RNA
aggagggaggagatgggccaagtt

SEQ ID NO: 219 - RNA, 24 nt, Homo sapiens
  source 1..24: mol_type = transcribed RNA
ctgcaggcagaagtggggctgaca

SEQ ID NO: 220 - RNA, 24 nt, Homo sapiens
  source 1..24: mol_type = transcribed RNA
agcctggaagctggagcctgcagt

SEQ ID NO: 221 - RNA, 22 nt, Homo sapiens
  source 1..22: mol_type = transcribed RNA
atagtgggaagctggcagattc

SEQ ID NO: 222 - RNA, 22 nt, Homo sapiens
  source 1..22: mol_type = transcribed RNA
cagttgggtctaggggtcagga

SEQ ID NO: 223 - RNA, 21 nt, Homo sapiens
  source 1..21: mol_type = transcribed RNA
agcagacttgacctacaatta

SEQ ID NO: 224 - RNA, 22 nt, Homo sapiens
  source 1..22: mol_type = transcribed RNA
caggcacgggagctcaggtgag

SEQ ID NO: 225 - RNA, 21 nt, Homo sapiens
  source 1..21: mol_type = transcribed RNA
agccaagtggaagttacttta

SEQ ID NO: 226 - RNA, 22 nt, Homo sapiens
  source 1..22: mol_type = transcribed RNA
aggactgatcctctcgggcagg

SEQ ID NO: 227 - RNA, 23 nt, Homo sapiens
  source 1..23: mol_type = transcribed RNA
acggggagtcaggcagtggtgga

SEQ ID NO: 228 - RNA, 20 nt, Homo sapiens
  source 1..20: mol_type = transcribed RNA
cagggggactgggggtgagc

SEQ ID NO: 229 - RNA, 21 nt, Homo sapiens
  source 1..21: mol_type = transcribed RNA
tggctgttggagggggcaggc

SEQ ID NO: 230 - RNA, 22 nt, Homo sapiens
  source 1..22: mol_type = transcribed RNA
aaggggcagggacgggtggccc

SEQ ID NO: 231 - RNA, 22 nt, Homo sapiens
  source 1..22: mol_type = transcribed RNA
ctcgggagggcatgggccaggc

SEQ ID NO: 232 - RNA, 19 nt, Homo sapiens
  source 1..19: mol_type = transcribed RNA
gagggcgggtggaggagga

SEQ ID NO: 233 - RNA, 23 nt, Homo sapiens
  source 1..23: mol_type = transcribed RNA
ttagggagtagaagggtggggag

SEQ ID NO: 234 - RNA, 19 nt, Homo sapiens
  source 1..19: mol_type = transcribed RNA
gggggaagaaaaggtgggg

SEQ ID NO: 235 - RNA, 17 nt, Homo sapiens
  source 1..17: mol_type = transcribed RNA
gtggggccaggcggtgg

SEQ ID NO: 236 - RNA, 18 nt, Homo sapiens
  source 1..18: mol_type = transcribed RNA
ccagaggtggggactgag

SEQ ID NO: 237 - RNA, 20 nt, Homo sapiens
  source 1..20: mol_type = transcribed RNA
tcagggagtcaggggagggc

SEQ ID NO: 238 - RNA, 17 nt, Homo sapiens
  source 1..17: mol_type = transcribed RNA
gggagaagggtcggggc

SEQ ID NO: 239 - RNA, 20 nt, Homo sapiens
  source 1..20: mol_type = transcribed RNA
cggggtgggtgaggtcgggc

SEQ ID NO: 240 - RNA, 22 nt, Homo sapiens
  source 1..22: mol_type = transcribed RNA
tggggaaggcgtcagtgtcggg

SEQ ID NO: 241 - RNA, 20 nt, Homo sapiens
  source 1..20: mol_type = transcribed RNA
gcggaaggcggagcggcgga

SEQ ID NO: 242 - RNA, 20 nt, Homo sapiens
  source 1..20: mol_type = transcribed RNA
tagggggtggcaggctggcc

SEQ ID NO: 243 - RNA, 22 nt, Homo sapiens
  source 1..22: mol_type = transcribed RNA
cccctggggctgggcaggcgga

SEQ ID NO: 244 - RNA, 22 nt, Homo sapiens
  source 1..22: mol_type = transcribed RNA
ttggggtggagggccaaggagc

SEQ ID NO: 245 - RNA, 19 nt, Homo sapiens
  source 1..19: mol_type = transcribed RNA
taaggagggggatgagggg

SEQ ID NO: 246 - RNA, 21 nt, Homo sapiens
  source 1..21: mol_type = transcribed RNA
gtgtggccggcaggcgggtgg

SEQ ID NO: 247 - RNA, 21 nt, Homo sapiens
  source 1..21: mol_type = transcribed RNA
ctggggggaggagaccctgct

SEQ ID NO: 248 - RNA, 22 nt, Homo sapiens
  source 1..22: mol_type = transcribed RNA
aaaagctgggttgagagggcga

SEQ ID NO: 249 - RNA, 22 nt, Homo sapiens
  source 1..22: mol_type = transcribed RNA
aggcggggcgccgcgggaccgc

SEQ ID NO: 250 - RNA, 23 nt, Homo sapiens
  source 1..23: mol_type = transcribed RNA
gggggggcaggaggggctcaggg

SEQ ID NO: 251 - RNA, 22 nt, Homo sapiens
  source 1..22: mol_type = transcribed RNA
agacacatttggagagggaccc

SEQ ID NO: 252 - RNA, 23 nt, Homo sapiens
  source 1..23: mol_type = transcribed RNA
gggggccgatacactgtacgaga

SEQ ID NO: 253 - RNA, 22 nt, Homo sapiens
  source 1..22: mol_type = transcribed RNA
acaggtgaggttcttgggagcc

SEQ ID NO: 254 - RNA, 23 nt, Homo sapiens
  source 1..23: mol_type = transcribed RNA
caacggaatcccaaaagcagctg

SEQ ID NO: 255 - RNA, 22 nt, Homo sapiens
  source 1..22: mol_type = transcribed RNA
agggacgggacgcggtgcagtg

SEQ ID NO: 256 - RNA, 21 nt, Homo sapiens
  source 1..21: mol_type = transcribed RNA
agggccccccctcaatcctgt

SEQ ID NO: 257 - RNA, 19 nt, Homo sapiens
  source 1..19: mol_type = transcribed RNA
aatggatttttggagcagg

SEQ ID NO: 258 - RNA, 22 nt, Homo sapiens
  source 1..22: mol_type = transcribed RNA
gggtggggatttgttgcattac

SEQ ID NO: 259 - RNA, 23 nt, Homo sapiens
  source 1..23: mol_type = transcribed RNA
cggggccgtagcactgtctgaga

SEQ ID NO: 260 - RNA, 19 nt, Homo sapiens
  source 1..19: mol_type = transcribed RNA
tggatttttggatcaggga

SEQ ID NO: 261 - RNA, 21 nt, Homo sapiens
  source 1..21: mol_type = transcribed RNA
gcagggacagcaaaggggtgc

SEQ ID NO: 262 - RNA, 22 nt, Homo sapiens
  source 1..22: mol_type = transcribed RNA
tgcggggctagggctaacagca

SEQ ID NO: 263 - RNA, 22 nt, Homo sapiens
  source 1..22: mol_type = transcribed RNA
tatagggattggagccgtggcg

SEQ ID NO: 264 - RNA, 22 nt, Homo sapiens
  source 1..22: mol_type = transcribed RNA
aaaccgttaccattactgagtt

SEQ ID NO: 265 - RNA, 22 nt, Homo sapiens
  source 1..22: mol_type = transcribed RNA
gactatagaactttccccctca

SEQ ID NO: 266 - RNA, 22 nt, Homo sapiens
  source 1..22: mol_type = transcribed RNA
tattgcacttgtcccggcctgt

SEQ ID NO: 267 - RNA, 22 nt, Homo sapiens
  source 1..22: mol_type = transcribed RNA
actggacttagggtcagaaggc

SEQ ID NO: 268 - RNA, 22 nt, Homo sapiens
  source 1..22: mol_type = transcribed RNA
agacacatttggagagggaacc

SEQ ID NO: 269 - RNA, 22 nt, Homo sapiens
  source 1..22: mol_type = transcribed RNA
aagacgggaggaaagaagggag

SEQ ID NO: 270 - RNA, 25 nt, Homo sapiens
  source 1..25: mol_type = transcribed RNA
caaccctaggagagggtgccattca

SEQ ID NO: 271 - RNA, 22 nt, Homo sapiens
  source 1..22: mol_type = transcribed RNA
tggctcagttcagcaggaacag

SEQ ID NO: 272 - RNA, 21 nt, Homo sapiens
  source 1..21: mol_type = transcribed RNA
atcacattgccagggattacc

SEQ ID NO: 273 - RNA, 21 nt, Homo sapiens
  source 1..21: mol_type = transcribed RNA
atcacattgccagggatttcc

SEQ ID NO: 274 - RNA, 22 nt, Homo sapiens
  source 1..22: mol_type = transcribed RNA
tattgcactcgtcccggcctcc

SEQ ID NO: 275 - RNA, 22 nt, Homo sapiens
  source 1..22: mol_type = transcribed RNA
aagctgccagttgaagaactgt

SEQ ID NO: 276 - RNA, 77 nt, Homo sapiens
  source 1..77: mol_type = transcribed RNA
catgagaaatcctgctggtcaaccatagccctggtcagactctccggggctgtgattgaccagcaggacttctcatg

SEQ ID NO: 277 - RNA, 82 nt, Homo sapiens
  source 1..82: mol_type = transcribed RNA
gggaaagcggagggcgcgcccagctcccgggctgattgcgctaacagtggccccggtgttggggcgcgtctgccgctgcccc

SEQ ID NO: 278 - RNA, 73 nt, Homo sapiens
  source 1..73: mol_type = transcribed RNA
gtgggcgggggcaggtgtgtggtgggtggtggcctgcggtgagcagggccctcacacctgcctcgccccccag

SEQ ID NO: 279 - RNA, 51 nt, Homo sapiens
  source 1..51: mol_type = transcribed RNA
acagaccccggggagcccggcggtgaagctcctggtatcctgggtgtctga

SEQ ID NO: 280 - RNA, 65 nt, Homo sapiens
  source 1..65: mol_type = transcribed RNA
gagggctaggtggggggcttgaagccccgagatgcctcacgtcttcacccctctcacctaagcag

SEQ ID NO: 281 - RNA, 67 nt, Homo sapiens
  source 1..67: mol_type = transcribed RNA
tacaggccggggctttgggtgagggacccccggagtctgtcacggtctcaccccaactctgccccag

SEQ ID NO: 282 - RNA, 102 nt, Homo sapiens
  source 1..102: mol_type = transcribed RNA
gcttatcgaggaaaagatcgaggtgggttggggcgggctctggggatttggtctcacagcccggatcccagcccacttaccttggttactctccttccttct

SEQ ID NO: 283 - RNA, 149 nt, Homo sapiens
  source 1..149: mol_type = transcribed RNA
catcaagacccagctgagtcactgtcactgcctaccaatctcgaccggacctcgaccggctcgtctgtgttgccaatcgactcggcgtggcgtcggtcgtggtagataggcggtcatgcatacgaattttcagctcttgttctggtgac

SEQ ID NO: 284 - RNA, 72 nt, Homo sapiens
  source 1..72: mol_type = transcribed RNA
gatttcagtgacctggcagcagggagcgtcgtcagtgtttgactgtttatggtatgtcagggagctggttcc

SEQ ID NO: 285 - RNA, 75 nt, Homo sapiens
  source 1..75: mol_type = transcribed RNA
aagcaagactgaggggcctcagaccgagcttttggaaaatagaaaagtctcgctctctgcccctcagcctaactt

SEQ ID NO: 286 - RNA, 82 nt, Homo sapiens
  source 1..82: mol_type = transcribed RNA
gtgagtgggaggccagggcacggcagggggagctgcagggctatgggaggggccccagcgtctgagccctgtcctcccgcag

SEQ ID NO: 287 - RNA, 82 nt, Homo sapiens; identical to SEQ ID NO: 286
  source 1..82: mol_type = transcribed RNA

SEQ ID NO: 288 - RNA, 51 nt, Homo sapiens
  source 1..51: mol_type = transcribed RNA
agagatgaagcgggggggcggggtcttgctctattgcctacgctgatctca

SEQ ID NO: 289 - RNA, 115 nt, Homo sapiens
  source 1..115: mol_type = transcribed RNA
gagcaaaaaccagagaacaacatgggagcgttcctaacccctaaggcaactggatgggagacctgacccatccagttctctgagggggctcttgtgtgttctacaaggttgttca

SEQ ID NO: 290 - RNA, 79 nt, Homo sapiens
  source 1..79: mol_type = transcribed RNA
aattaatccctctctttctagttcttcctagagtgaggaaaagctgggttgagagggcaaacaaattaactaattaatt

SEQ ID NO: 291 - RNA, 138 nt, Homo sapiens
  source 1..138: mol_type = transcribed RNA
tgttattttttgtcttctacctaagaattctgtctcttaggctttctcttcccagatttcccaaagttgggaaaagctgggttgagagggcaaaaggaaaaaaaaagaattctgtctctgacataattagatagggaa

SEQ ID NO: 292 - RNA, 64 nt, Homo sapiens
  source 1..64: mol_type = transcribed RNA
tctgggcgaggggtgggctctcagaggggctggcagtactgctctgaggcctgcctctccccag

SEQ ID NO: 293 - RNA, 82 nt, Homo sapiens
  source 1..82: mol_type = transcribed RNA
acctgtaggtgacagtcaggggcggggtgtggtggggctggggctggccccctcctcacacctctcctggcatcgcccccag

SEQ ID NO: 294 - RNA, 84 nt, Homo sapiens
  source 1..84: mol_type = transcribed RNA
gctggcgtcggtgctggggagcggcccccgggtgggcctctgctctggcccctcctggggcccgcactctcgctctgggcccgc

SEQ ID NO: 295 - RNA, 76 nt, Homo sapiens
  source 1..76: mol_type = transcribed RNA
cgcaggcctctggcggagcccattccatgccagatgctgagcgatggctggtgtgtgctgctccacaggcctggtg

SEQ ID NO: 296 - RNA, 66 nt, Homo sapiens
  source 1..66: mol_type = transcribed RNA
cctggaggggggcactgcgcaagcaaagccagggaccctgagaggctttgcttcctgctcccctag

SEQ ID NO: 297 - RNA, 119 nt, Homo sapiens
  source 1..119: mol_type = transcribed RNA
ccatgaggagctggcagtgggatggcctgggggtaggagcgtggcttctggagctagaccacatgggttcagatcccagcggtgcctctaactggccacaggaccttgggcagtcagct

SEQ ID NO: 298 - RNA, 85 nt, Homo sapiens
  source 1..85: mol_type = transcribed RNA
tcctccccggagccaggatgcagctcaagccacagcagggtgtttagcgctcttcagtggctccagattgtggcgctggtgcagg

SEQ ID NO: 299 - RNA, 63 nt, Homo sapiens
  source 1..63: mol_type = transcribed RNA
accctagggtggggctggaggtggggctgaggctgagtcttcctccccttcctccctgcccag

SEQ ID NO: 300 - RNA, 69 nt, Homo sapiens
  source 1..69: mol_type = transcribed RNA
gggatactcaaaatgggggcgctttcctttttgtctgtactgggaagtgcttcgattttggggtgtccc

SEQ ID NO: 301 - RNA, 52 nt, Homo sapiens
  source 1..52: mol_type = transcribed RNA
tagccgggcgtggtggtgggggcctgtggtcccagctactttggaggctgag

SEQ ID NO: 302 - RNA, 89 nt, Homo sapiens
  source 1..89: mol_type = transcribed RNA
tctccgtttatcccaccactgccaccattattgctactgttcagcaggtgctgctggtggtgatggtgatagtctggtgggggcggtgg

SEQ ID NO: 303 - RNA, 91 nt, Homo sapiens
  source 1..91: mol_type = transcribed RNA
acgccccccgccccgccaccgccttggaggctgacctcttactttcggtcggtcttcttccctgggcttggtttgggggcgggggagtgtc

SEQ ID NO: 304 - RNA, 78 nt, Homo sapiens
  source 1..78: mol_type = transcribed RNA
gggcccagaagggggcgcagtcactgacgtgaagggaccacatcccgcttcatgtcagtgactcctgccccttggtct

SEQ ID NO: 305 - RNA, 47 nt, Homo sapiens
  source 1..47: mol_type = transcribed RNA
ctcggcgcggggcgcgggctccgggttggggcgagccaacgccgggg

SEQ ID NO: 306 - RNA, 81 nt, Homo sapiens
  source 1..81: mol_type = transcribed RNA
gcgggaggtgtaacaggactggactcccggcagccccagggcaggggcgtggggagctggtcctagctcagcgctcccgga

SEQ ID NO: 307 - RNA, 64 nt, Homo sapiens
  source 1..64: mol_type = transcribed RNA
gaggcactgggtaggtggggctccagggctcctgacacctggacctctcctccccaggcccaca

SEQ ID NO: 308 - RNA, 74 nt, Homo sapiens
  source 1..74: mol_type = transcribed RNA
gtgcgtggtggctcgaggcgggggtgggggcctcgccctgcttgggccctccctgacctctccgctccgcacag

SEQ ID NO: 309 - RNA, 94 nt, Homo sapiens
  source 1..94: mol_type = transcribed RNA
aattcagccctgccactggcttatgtcatgaccttgggctactcaggctgtctgcacaatgagccagttggacaggagcagtgccactcaactc

SEQ ID NO: 310 - RNA, 62 nt, Homo sapiens
  source 1..62: mol_type = transcribed RNA
tggcctagggggcggcttgtggagtgtatgggctgagccttgctctgctcccccgcccccag

SEQ ID NO: 311 - RNA, 71 nt, Homo sapiens
  source 1..71: mol_type = transcribed RNA
ggtgagtgggagccggtggggctggagtaagggcacgcccggggctgccccacctgctgaccaccctcccc

SEQ ID NO: 312 - RNA, 97 nt, Homo sapiens
  source 1..97: mol_type = transcribed RNA
cccgggaccttggtccaggcgctggtctgcgtggtgctcgggtggataagtctgatctgagcaccacacaggccgggcgccgggaccaagggggctc

SEQ ID NO: 313 - RNA, 56 nt, Homo sapiens
  source 1..56: mol_type = transcribed RNA
cgaccgcacccgcccgaagctgggtcaaggagcccagcaggacgggagcgcggcgc

SEQ ID NO: 314 - RNA, 67 nt, Homo sapiens
  source 1..67: mol_type = transcribed RNA
ggcagccagggggatgggcgagcttgggcccattcctttccttaccctaccccccatccccctgtag

SEQ ID NO: 315 - RNA, 64 nt, Homo sapiens
  source 1..64: mol_type = transcribed RNA
aaccccgggccggaggtcaagggcgtcgcttctccctaatgttgcctcttttccacggcctcag

SEQ ID NO: 316 - RNA, 74 nt, Homo sapiens
  source 1..74: mol_type = transcribed RNA
ccgcactctctccattacactaccctgcctcttctccatgagaggcagcggggtgtagtggatagagcacgggt

SEQ ID NO: 317 - RNA, 100 nt, Homo sapiens
  source 1..100: mol_type = transcribed RNA
gaggtgggaggattgcttgagtcagggtggttgaggctgcagtaagttgtgatcataccactgcactccagcctgagtgacagagcaagaccttgtctca

SEQ ID NO: 318 - RNA, 84 nt, Homo sapiens
  source 1..84: mol_type = transcribed RNA
cggtccagacgtggcgggggtggcggcggcatcccggacggcctgtgagggatgcgccgcccactgccccgcgccgcctgaccg

SEQ ID NO: 319 - RNA, 55 nt, Homo sapiens
  source 1..55: mol_type = transcribed RNA
ccggatccgagtcacggcaccaaatttcatgcgtgtccgtgtgaagagaccacca

SEQ ID NO: 320 - RNA, 82 nt, Homo sapiens
  source 1..82: mol_type = transcribed RNA
gctacggggagcggggaggaagtgggcgctgcttctgcgttatctggaaggagcagcccactcctgtcctgggctctgtggt

SEQ ID NO: 321 - RNA, 66 nt, Homo sapiens
  source 1..66: mol_type = transcribed RNA
gggaggagggaggagatgggccaagttccctctggctggaacgcccttcccccccttcttcacctg

SEQ ID NO: 322 - RNA, 79 nt, Homo sapiens
  source 1..79: mol_type = transcribed RNA
gtgcagatccttgggagccctgttagactctggattttacacttggagtgaacgggcgccatcccgaggctttgcacag

SEQ ID NO: 323 - RNA, 113 nt, Homo sapiens
  source 1..113: mol_type = transcribed RNA
gccgggtggggcggggcggcctcaggaggggcccagctcccctggatgtgctgcggtggggccggaggggcgtcacgtgcacccaagtgacgccccttctgattctgcctcag

SEQ ID NO: 324 - RNA, 83 nt, Homo sapiens
  source 1..83: mol_type = transcribed RNA
gtgagtgggagccccagtgtgtggttggggccatggcgggtgggcagcccagcctctgagccttcctcgtctgtctgccccag

SEQ ID NO: 325 - RNA, 69 nt, Homo sapiens
  source 1..69: mol_type = transcribed RNA
ggccctcgggcctggggttgggggagctctgtcctgtctcactcattgctcctcccctgcctggcccag

SEQ ID NO: 326 - RNA, 65 nt, Homo sapiens
  source 1..65: mol_type = transcribed RNA
gagggtgggcgagggcggctgagcggctccatcccccggcctgctcatccccctcgccctctcag

SEQ ID NO: 327 - RNA, 66 nt, Homo sapiens
  source 1..66: mol_type = transcribed RNA
gggcatggggaggtgtggagtcagcatggggctaggaggccccgcgctgacccgccttctccgcag

SEQ ID NO: 328 - RNA, 115 nt, Homo sapiens
  source 1..115: mol_type = transcribed RNA
ggtgccgagggccgtccggcatcctaggcgggtcgctgcggtacctccctcctgtctgtggcggtgggatcccgtggccgtgttttcctggtggcccggccgtgcctgaggtttc

SEQ ID NO: 329 - RNA, 67 nt, Homo sapiens
  source 1..67: mol_type = transcribed RNA
gtgaggaggggctggcagggacccctccaagttggggacggcagccagcccctgctcacccctcgcc

SEQ ID NO: 330 - RNA, 60 nt, Homo sapiens
  source 1..60: mol_type = transcribed RNA
gagcaggcgaggctgggctgaacccgtgggtgaggagtgcagcccagctgaggcctctgc

SEQ ID NO: 331 - RNA, 87 nt, Homo sapiens
  source 1..87: mol_type = transcribed RNA
gtgaggcggggccaggagggtgtgtggcgtgggtgctgcggggccgtcagggtgcctgcgggacgctcacctggctggcccgcccag

SEQ ID NO: 332 - RNA, 56 nt, Homo sapiens
  source 1..56: mol_type = transcribed RNA
gaccgagtggggtgagggcaggtggttcttcccgaagcagctctcgcctcttcgtc

SEQ ID NO: 333 - RNA, 69 nt, Homo sapiens
  source 1..69: mol_type = transcribed RNA
gaacctcggggcatgggggagggaggctggacaggagagggctcacccaggccctgtcctctgccccag

SEQ ID NO: 334 - RNA, 67 nt, Homo sapiens
  source 1..67: mol_type = transcribed RNA
gtggcactcaaactgtgggggcactttctgctctctggtgaaagtgccgccatcttttgagtgttac

SEQ ID NO: 335 - RNA, 66 nt, Homo sapiens
  source 1..66: mol_type = transcribed RNA
ccgagtggggcggggcaggtccctgcagggactgtgacactgaaggacctgcaccttcgcccacag

SEQ ID NO: 336 - RNA, 80 nt, Homo sapiens
  source 1..80: mol_type = transcribed RNA
aggacccttccagagggccccccctcaatcctgttgtgcctaattcagagggttgggtggaggctctcctgaagggctct

SEQ ID NO: 337 - RNA, 49 nt, Homo sapiens
  source 1..49: mol_type = transcribed RNA
ttcccagccaacgcaccaaaaatgatatgggtctgttgtctggagaaac

SEQ ID NO: 338 - RNA, 86 nt, Homo sapiens
  source 1..86: mol_type = transcribed RNA
cgcctgagcgtgcagcaggacatcttcctgacctggtaataattaggtgagaaggatggttgggggcggtcggcgtaactcaggga

SEQ ID NO: 339 - RNA, 86 nt, Homo sapiens; identical to SEQ ID NO: 338
  source 1..86: mol_type = transcribed RNA

SEQ ID NO: 340 - RNA, 57 nt, Homo sapiens
  source 1..57: mol_type = transcribed RNA
cattggagggtgtggaagacatctgggccaactctgatctcttcatctaccccccag

SEQ ID NO: 341 - RNA, 80 nt, Homo sapiens
  source 1..80: mol_type = transcribed RNA
acctgaggagccagccctcctcccgcacccaaacttggagcacttgacctttggctgttggagggggcaggctcgcgggt

SEQ ID NO: 342 - RNA, 86 nt, Homo sapiens
  source 1..86: mol_type = transcribed RNA
ggggctgggggtgtggggagagagagtgcacagccagctcagggattaaagctctttctctctctctctctcccacttccctgcag

SEQ ID NO: 343 - RNA, 75 nt, Homo sapiens
  source 1..75: mol_type = transcribed RNA
gtcagaggggggatgtgcatgctggttggggtgggctgcctgtggaccaatcagcgtgcacttccccaccctgaa

SEQ ID NO: 344 - RNA, 81 nt, Homo sapiens
  source 1..81: mol_type = transcribed RNA
attcaggccggtcctgcagagaggaagcccttctgcttacaggtattggaagggcttcctctctgcaggaccggcctgaat

SEQ ID NO: 345 - RNA, 81 nt, Homo sapiens
  source 1..81: mol_type = transcribed RNA
attcaggccggtcctgcagagaggaagcccttccaatacctgtaagcagaagggcttcctctctgcaggaccggcctgaat

SEQ ID NO: 346 - RNA, 61 nt, Homo sapiens
  source 1..61: mol_type = transcribed RNA
tcggctggcgggggtagagctggctgcaggcccggcccctctcagctgctgccctctccag

SEQ ID NO: 347 - RNA, 90 nt, Homo sapiens
  source 1..90: mol_type = transcribed RNA
tctaagaaacgcagtggtctctgaagcctgcaggggcaggccagccctgcactgaacgcctgttcttgccaggtggcagaaggttgctgc

SEQ ID NO: 348 - RNA, 70 nt, Homo sapiens
  source 1..70: mol_type = transcribed RNA
ggtttctccttgaggagacatggtgggggccggtcaggcagcccatgccatgtgtcctcatggagaggcc

SEQ ID NO: 349 - RNA, 86 nt, Homo sapiens
  source 1..86: mol_type = transcribed RNA
tttggtacttaaagagaggataccctttgtatgttcacttgattaatggcgaatatacagggggagactctcatttgcgtatcaaa

SEQ ID NO: 350 - RNA, 50 nt, Homo sapiens
  source 1..50: mol_type = transcribed RNA
acccgggcgtggtggtgggggtgggtgcctgtaattccagctagttggga

SEQ ID NO: 351 - RNA, 86 nt, Homo sapiens
  source 1..86: mol_type = transcribed RNA
tttggtacttgaagagaggataccctttgtatgttcacttgattaatggcgaatatacagggggagactcttatttgcgtatcaaa

SEQ ID NO: 352 - RNA, 94 nt, Homo sapiens
  source 1..94: mol_type = transcribed RNA
gtgaggtgtgggcccggccccaggagcggggcctgggcagccccgtgtgttgaggaaggaaggcagggcccccgctccccgggcctgaccccac

SEQ ID NO: 353 - RNA, 82 nt, Homo sapiens
  source 1..82: mol_type = transcribed RNA
tgtgggcagggccctggggagctgaggctctgggggtggccggggctgaccctgggcctctgctccccagtgtctgaccgcg

SEQ ID NO: 354 - RNA, 69 nt, Homo sapiens
  source 1..69: mol_type = transcribed RNA
gggcttagggatgggaggccaggatgaagattaatccctaatccccaacactggccttgctatccccag

SEQ ID NO: 355 - RNA, 80 nt, Homo sapiens
  source 1..80: mol_type = transcribed RNA
cctctgtgagaaagggtgtgggggagaggctgtcttgtgtctgtaagtatgccaaacttattttccccaaggcagaggga

SEQ ID NO: 356 - RNA, 100 nt, Homo sapiens
  source 1..100: mol_type = transcribed RNA
agctcagggcggctgcgcagagggctggactcagcggcggagctggctgctggcctcagttctgcctctgtccaggtccttgtgacccgcccgctctcct

SEQ ID NO: 357 - RNA, 98 nt, Homo sapiens
  source 1..98: mol_type = transcribed RNA
cttggtcaataggaaagaggtgggacctcctggcttttcctctgcagcatggctcggacctagtgcaatgtttaagctcccctctctttcctgttcag

SEQ ID NO: 358 - RNA, 68 nt, Homo sapiens
  source 1..68: mol_type = transcribed RNA
cgtggtgaggatatggcagggaaggggagtttccctctattcccttccccccagtaatcttcatcatg

SEQ ID NO: 359 - RNA, 51 nt, Homo sapiens
  source 1..51: mol_type = transcribed RNA
agctgtacctgaaaccaagcacctgtttgtgacttggcttcagttactagc

SEQ ID NO: 360 - RNA, 93 nt, Homo sapiens
  source 1..93: mol_type = transcribed RNA
tacttatggcaccccactcctggtaccatagtcataagttaggagatgttagagctgtgagtaccatgacttaagtgtggtggcttaaacatg

SEQ ID NO: 361 - RNA, 81 nt, Homo sapiens
  source 1..81: mol_type = transcribed RNA
tacaggtgcaggggaactgtagatgaaaaggcttggcacttgagggaaagcctcagttcattctcattttgctcacctgtt

SEQ ID NO: 362 - RNA, 91 nt, Homo sapiens
  source 1..91: mol_type = transcribed RNA
gttctagagcatggtttctcatcatttgcactactgatacttggggtcagataattgtttgtggtgggggctgttgtttgcattgtaggat

SEQ ID NO: 363 - RNA, 86 nt, Homo sapiens
  source 1..86: mol_type = transcribed RNA
aggagtgaccaaaagacaagagtgcgagccttctattatgcccagacagggccaccagagggctccttggtctaggggtaatgcca

SEQ ID NO: 364 - RNA, 100 nt, Homo sapiens
  source 1..100: mol_type = transcribed RNA
gctcggttgccgtggttgcgggccctgcccgcccgccagctcgctgacagcacgactcagggcggagggaagtaggtccgttggtcggtcgggaacgagg

SEQ ID NO: 365 - RNA, 72 nt, Homo sapiens
  source 1..72: mol_type = transcribed RNA
atgagcgggtgggagcagatcttattgagagttccttctcctgctcctgattgtcttcccccaccctcacag

SEQ ID NO: 366 - RNA, 75 nt, Homo sapiens
  source 1..75: mol_type = transcribed RNA
ggctgtgccgggtagagagggcagtgggaggtaagagctcttcacccttcaccaccttctccacccagcatggcc

SEQ ID NO: 367 - RNA, 65 nt, Homo sapiens
  source 1..65: mol_type = transcribed RNA
gagaatggggggacagatggagaggacacaggctggcactgaggtcccctccactttcctcctag

SEQ ID NO: 368 - RNA, 62 nt, Homo sapiens
  source 1..62: mol_type = transcribed RNA
gagggagtggggtgggacccagctgttggccatggcgacaacacctgggttgtcccctctag

SEQ ID NO: 369 - RNA, 85 nt, Homo sapiens
  source 1..85: mol_type = transcribed RNA
tctgaggtacccggggcagattggtgtagggtgcaaagcctgcccgccccctaagccttctgcccccaactccagcctgtcagga

SEQ ID NO: 370 - RNA, 68 nt, Homo sapiens
  source 1..68: mol_type = transcribed RNA
gtgcaaagagcaggaggacaggggatttatctcccaagggaggtcccctgatcctagtcacggcacca

SEQ ID NO: 371 - RNA, 73 nt, Homo sapiens
  source 1..73: mol_type = transcribed RNA
ggaggctgggctgggacggacacccggcctccactttctgtggcaggtacctcctccatgtcggcccgccttg

SEQ ID NO: 372 - RNA, 59 nt, Homo sapiens
  source 1..59: mol_type = transcribed RNA
ctgtgtcggggagtctggggtccggaattctccagagcctctgtgcccctacttcccag

SEQ ID NO: 373 - RNA, 67 nt, Homo sapiens
  source 1..67: mol_type = transcribed RNA
tttaggagagagatgccgccttgctccttgaacaggaggagcaaggcggcatctctctgatactaaa

SEQ ID NO: 374 - RNA, 109 nt, Homo sapiens
  source 1..109: mol_type = transcribed RNA
tcccgcattccctctgctttggtcaggtggtgccctccttccatgggtagagccagagatggtgggttctggctggtcagatgggagtggacagagacccggggtcctc

SEQ ID NO: 375 - RNA, 90 nt, Homo sapiens
  source 1..90: mol_type = transcribed RNA
gtgggtacggcccagtgggggggagagggacacgccctgggctctgcccagggtgcagccggactgactgagcccctgtgccgcccccag

SEQ ID NO: 376 - RNA, 109 nt, Homo sapiens
  source 1..109: mol_type = transcribed RNA
tgctattgtcttactgctacagcagggctggggattgcagtatccgctgttgctgctgctcccagtcctgcccctgctgctacctagtccagcctcaccgcatcccaga

SEQ ID NO: 377 - RNA, 70 nt, Homo sapiens
  source 1..70: mol_type = transcribed RNA
ctcgggcccgaccgcgccggcccgcacctcccggcccggagctgcgggctgcggtcagggcgatcccggg

SEQ ID NO: 378 - RNA, 73 nt, Homo sapiens
  source 1..73: mol_type = transcribed RNA
aggtggcagggccagccaccaggagggctgcgtgccacccgggcagctctgctgctcactggcagtgtcacct

SEQ ID NO: 379 - RNA, 100 nt, Homo sapiens
  source 1..100: mol_type = transcribed RNA
gtgagcgggcgcggcagggatcgcgggcgggtggcggcctagggcgcggagggcggaccgggaatggcgcgccgtgcgccgccggcgtaactgcggcgct

SEQ ID NO: 380 - RNA, 81 nt, Homo sapiens
  source 1..81: mol_type = transcribed RNA
ggacaggcacctgaggctctgttagccttggctctgggtcctgctccttagagcagaggcagagaggctcagggtctgtct

SEQ ID NO: 381 - RNA, 67 nt, Homo sapiens
  source 1..67: mol_type = transcribed RNA
gtgggaggggagaggcagcaagcacacagggcctgggactagcatgctgacctccctcctgccccag

SEQ ID NO: 382 - RNA, 137 nt, Homo sapiens
  source 1..137: mol_type = transcribed RNA
agccagacaagagggtcatggggagtcactgtcaacccagagcaggcactgcccctgcgaccagcctggggcatcggttggggtgcaggggtctgctggtgatgctttccatctctttgctttgtcctgattgtagc

SEQ ID NO: 383 - RNA, 98 nt, Homo sapiens
  source 1..98: mol_type = transcribed RNA
cgaggtaggggcgtcccgggcgcgcgggcgggtcccaggctgggcccctcggaggccgggtgctcactgccccgtcccggcgcccgtgtctcctccag

SEQ ID NO: 384 - RNA, 81 nt, Homo sapiens
  source 1..81: mol_type = transcribed RNA
tagaggcagtttcaacagatgtgtagacttttgatatgagaaattggtttcaaaatcaggagtcggggctttactgctttt

SEQ ID NO: 385 - RNA, 73 nt, Homo sapiens
  source 1..73: mol_type = transcribed RNA
agggagaaaagctgggctgagaggcgactggtgtctaatttgtttgtctctccaactcagactgcctggccca

SEQ ID NO: 386 - RNA, 71 nt, Homo sapiens
  source 1..71: mol_type = transcribed RNA
tgaccacccccgggcaaagacctgcagatcccctgttagagacgggcccaggactttgtgcggggtgccca

SEQ ID NO: 387 - RNA, 70 nt, Homo sapiens
  source 1..70: mol_type = transcribed RNA
aaaagcctgtccctaagtccctcccagccttccagagttggtgccaggaaggatttagggacaggctttg

SEQ ID NO: 388 - RNA, 73 nt, Homo sapiens
  source 1..73: mol_type = transcribed RNA
cccagggtctggtgcggagagggcccacagtggacttggtgacgctgtatgccctcaccgctcagcccctggg

SEQ ID NO: 389 - RNA, 76 nt, Homo sapiens
  source 1..76: mol_type = transcribed RNA
actgactttgagtctctcctcagggtgctgcaggcaaagctggggacccagggagagacgtaagtgaggggagatg

SEQ ID NO: 390 - RNA, 72 nt, Homo sapiens
  source 1..72: mol_type = transcribed RNA
gagtctgagggacccaggacaggagaaggcctatggtgatttgcattcttcctgccctggctccatcctcag

SEQ ID NO: 391 - RNA, 85 nt, Homo sapiens
  source 1..85: mol_type = transcribed RNA
ggacctgccctgggctttctagtctcagctctcctccagctcagctggtcaggagagctgagactagaaagcccagggcaggttc

SEQ ID NO: 392 - RNA, 81 nt, Homo sapiens
  source 1..81: mol_type = transcribed RNA
acctgccctgggctttctagtctcagctctcctgaccagctgagctggaggagagctgagactagaaagcccagggcaggt

SEQ ID NO: 393 - RNA, 80 nt, Homo sapiens
  source 1..80: mol_type = transcribed RNA
cgggaatgccgcggcggggacggcgattggtccgtatgtgtggtgccaccggccgccggctccgccccggcccccgcccc

SEQ ID NO: 394 - RNA, 61 nt, Homo sapiens
  source 1..61: mol_type = transcribed RNA
gggggcgggagctggggtctgcaggttcgcactgatgcctgctcgccctgtctcccgctag

SEQ ID NO: 395 - RNA, 80 nt, Homo sapiens
  source 1..80: mol_type = transcribed RNA
acctctacctcccggcagaggaggctgcagaggctggctttccaaaactctgccccctccgctgctgccaagtggctggt

SEQ ID NO: 396 - RNA, 65 nt, Homo sapiens
  source 1..65: mol_type = transcribed RNA
cggccacatggcccaggctcttctccgagtgatctcggtggactggagtgggtggtaggtggcag

SEQ ID NO: 397 - RNA, 65 nt, Homo sapiens; identical to SEQ ID NO: 396
  source 1..65: mol_type = transcribed RNA

SEQ ID NO: 398 - RNA, 180 nt, Homo sapiens
  source 1..180: mol_type = transcribed RNA
cgcgactgcggcggcggtggtggggggagccgcggggatcgccgagggccggtcggccgccccgggtgccgcgcggtgccgccggcggcggtgaggccccgcgcgtgtgtcccggctgcggtcggccgcgctcgaggggtccccgtggcgtccccttccccgccggccgcctttctcgcg

SEQ ID NO: 399 - RNA, 180 nt, Homo sapiens; identical to SEQ ID NO: 398
  source 1..180: mol_type = transcribed RNA

SEQ ID NO: 400 - RNA, 76 nt, Homo sapiens
  source 1..76: mol_type = transcribed RNA
gagggagctgtagagcagggagcaggaagctgtgtgtgtccagccctgacctgtcctgttctgcccccagcccctc

SEQ ID NO: 401 - RNA, 93 nt, Homo sapiens
  source 1..93: mol_type = transcribed RNA
ggcgcttttgtgcgcgcccgggtctgttggtgctcagagtgtggtcaggcggctcggactgagcaggtgggtgcggggctcggaggaggcggc

SEQ ID NO: 402 - RNA, 99 nt, Homo sapiens
  source 1..99: mol_type = transcribed RNA
cgcccacctcagcctcccaaaatgctgggattacaggcatgagccactgcggtcgaccatgacctggacatgtttgtgcccagtactgtcagtttgcag

SEQ ID NO: 403 - RNA, 92 nt, Homo sapiens
  source 1..92: mol_type = transcribed RNA
gtcagtgtctgggcggacagctgcaggaaagggaagaccaaggcttgctgtctgtccagtctgccaccctaccctgtctgttcttgccacag

SEQ ID NO: 404 - RNA, 99 nt, Homo sapiens
  source 1..99: mol_type = transcribed RNA
gaaactgggctcaaggtgaggggtgctatctgtgattgagggacatggttaatggaattgtctcacacagaaatcgcacccgtcaccttggcctactta

SEQ ID NO: 405 - RNA, 81 nt, Homo sapiens
  source 1..81: mol_type = transcribed RNA
catcctccttacgtcccaccccccactcctgtttctggtgaaatattcaaacaggagtgggggtgggacataaggaggata

SEQ ID NO: 406 - RNA, 80 nt, Homo sapiens
  source 1..80: mol_type = transcribed RNA
ggttccggagccccggcgcgggcgggttctggggtgtagacgctgctggccagcccgccccagccgaggttctcggcacc

SEQ ID NO: 407 - RNA, 61 nt, Homo sapiens
  source 1..61: mol_type = transcribed RNA
tccgctctgtggagtggggtgcctgtcccctgccactgggtgacccacccctctccaccag

SEQ ID NO: 408 - RNA, 62 nt, Homo sapiens
  source 1..62: mol_type = transcribed RNA
gtgggtctcgcatcaggaggcaaggccaggacccgctgacccatgcctcctgccgcggtcag

SEQ ID NO: 409 - RNA, 79 nt, Homo sapiens
  source 1..79: mol_type = transcribed RNA
cagcctggggaaggcttggcagggaagacacatgagcagtgcctccacttcacgcctctcccttgtctcctttccctag

SEQ ID NO: 410 - RNA, 99 nt, Homo sapiens
  source 1..99: mol_type = transcribed RNA
gcaagggacagggagggtcgtggcgacactcgcgccagctcccgggacggctgggctcgggctggtcgccgacctccgaccctccactagatgcctggc

SEQ ID NO: 411 - RNA, 72 nt, Homo sapiens
  source 1..72: mol_type = transcribed RNA
tctcctcgaggggtctctgcctctacccaggactctttcatgaccaggaggctgaggcccctcacaggcggc

SEQ ID NO: 412 - RNA, 70 nt, Homo sapiens
  source 1..70: mol_type = transcribed RNA
gtccctgggggctgggatgggccatggtgtgctctgatccccctgtggtctcttggcccccaggaactcc

SEQ ID NO: 413 - RNA, 94 nt, Homo sapiens
  source 1..94: mol_type = transcribed RNA
gggctgacccctagggtcaggtgaggcccttggggcacagtggtgccatctcccctgtgctcccagggcctcgcctgtcccttgaggtcggccc

SEQ ID NO: 414 - RNA, 76 nt, Homo sapiens
  source 1..76: mol_type = transcribed RNA
ccgatgcctcgggagtctacagcagggccatgtctgtgagggcccaagggtgcatgtgtctcccaggtttcggtgc

SEQ ID NO: 415 - RNA, 67 nt, Homo sapiens
  source 1..67: mol_type = transcribed RNA
cctgcaggcagaagtggggctgacagggcagagggttgcgccccctcaccatcccttctgcctgcag

SEQ ID NO: 416 - RNA, 67 nt, Homo sapiens; identical to SEQ ID NO: 415
  source 1..67: mol_type = transcribed RNA

SEQ ID NO: 417 - RNA, 67 nt, Homo sapiens; identical to SEQ ID NO: 415
  source 1..67: mol_type = transcribed RNA

SEQ ID NO: 418 - RNA, 67 nt, Homo sapiens; identical to SEQ ID NO: 415
  source 1..67: mol_type = transcribed RNA

SEQ ID NO: 419 - RNA, 80 nt, Homo sapiens
  source 1..80: mol_type = transcribed RNA
aggcagcaaatggccagagctcacacagagggatgagtgcacttcacctgcagtgtgactcagcaggccaacagatgcta

SEQ ID NO: 420 - RNA, 74 nt, Homo sapiens
  source 1..74: mol_type = transcribed RNA
gcaaatggccagagctcacacagagggatgagtgcacttcacctgcagtgtgactcagcaggccaacagatgct

SEQ ID NO: 421 - RNA, 54 nt, Homo sapiens
  source 1..54: mol_type = transcribed RNA
agggaaggaggcttggtcttagcacggggtctaaggcccgggctttcctcccag

SEQ ID NO: 422 - RNA, 62 nt, Homo sapiens
  source 1..62: mol_type = transcribed RNA
gagggtggtggaggaagagggcagctcccatgactgcctgaccgccttctctcctcccccag

SEQ ID NO: 423 - RNA, 62 nt, Homo sapiens
  source 1..62: mol_type = transcribed RNA
gtgagtagtggcgcgcggcggctcggagtacctctgccgccgcgcgcatcggctcagcatgc

SEQ ID NO: 424 - RNA, 94 nt, Homo sapiens
  source 1..94: mol_type = transcribed RNA
cacggtgtcccctggtggaacctggcagggggagaggtaaggtctttcagcctctccaaagcccatggtcaggtactcaggtgggggagccctg

SEQ ID NO: 425 - RNA, 73 nt, Homo sapiens
  source 1..73: mol_type = transcribed RNA
acctttccagctcatcccacctctgccaccaaaacactcatcgcggggtcagagggagtgccaaaaaaggtaa

SEQ ID NO: 426 - RNA, 64 nt, Homo sapiens
  source 1..64: mol_type = transcribed RNA
agttcagggccgaagggtggaagctgctggtgctcatctcagcctctgcccttggcctccccag

SEQ ID NO: 427 - RNA, 87 nt, Homo sapiens
  source 1..87: mol_type = transcribed RNA
ccctcatctctgggcaggggcttattgtaggagtctctgaagagagctgtggactgacctgctttaacccttccccaggttcccatt

SEQ ID NO: 428 - RNA, 75 nt, Homo sapiens
  source 1..75: mol_type = transcribed RNA
aggctggcgtgggctgagggcaggaggcctgtggccggtcccaggcctcctgcttcctgggctcaggctcggttt

SEQ ID NO: 429 - RNA, 69 nt, Homo sapiens
  source 1..69: mol_type = transcribed RNA
gtgggtagggtttgggggagagcgtgggctggggttcagggacaccctctcaccactgccctcccacag

SEQ ID NO: 430 - RNA, 91 nt, Homo sapiens
  source 1..91: mol_type = transcribed RNA
ggggcatttagggtaactgagctgctgccggggcctggcgctcctctaccttgtcaggtgacccagcagtccctccccctgcatggtgccc

SEQ ID NO: 431 - RNA, 68 nt, Homo sapiens
  source 1..68: mol_type = transcribed RNA
ctcaggctcagtggtgcatgcttatagtcccagccactctggaggctgaaggaagatggcttgagcct

SEQ ID NO: 432 - RNA, 87 nt, Homo sapiens
  source 1..87: mol_type = transcribed RNA
cccaggcgcccgctcccgacccacgccgcgccgccgggtccctcctccccggagaggctgggctcgggacgcgcggctcagctcggg

SEQ ID NO: 433 - RNA, 69 nt, Homo sapiens
  source 1..69: mol_type = transcribed RNA
ccgggcaggcaggtgtagggtggagcccactgtggctcctgactcagccctgctgccttcacctgccag

SEQ ID NO: 434 - RNA, 65 nt, Homo sapiens
  source 1..65: mol_type = transcribed RNA
ttctcctggggagtggctggggagcagacagacccaacctcatgctccccggcctctgcccccag

SEQ ID NO: 435 - RNA, 86 nt, Homo sapiens
  source 1..86: mol_type = transcribed RNA
agagccggggccatggagcagcctgtgtagacggggacctgccctgcatgggcaccccctcactggctgcttcccttggtctccag

SEQ ID NO: 436 - RNA, 64 nt, Homo sapiens
  source 1..64: mol_type = transcribed RNA
gaaggcgaggggtagaagagcacaggggttctgataaacccttctgcctgcattctactcccag

SEQ ID NO: 437 - RNA, 86 nt, Homo sapiens
  source 1..86: mol_type = transcribed RNA
attctaggtggggagactgacggctggaggcccataagctgtctaaaacttcggcccccagatttctggtctccccacttcagaac

SEQ ID NO: 438 - RNA, 63 nt, Homo sapiens
  source 1..63: mol_type = transcribed RNA
cttgcccgggagaaggaggtggcctggagagctgctgtctccagccgccgcctgtctccacag

SEQ ID NO: 439 - RNA, 47 nt, Homo sapiens
  source 1..47: mol_type = transcribed RNA
aggcaggttatctgggctgccatctcccactggctgcttgcctgcct

SEQ ID NO: 440 - RNA, 70 nt, Homo sapiens
  source 1..70: mol_type = transcribed RNA
ggccgcggcgcgcaagatggcggcgggcccgggcaccgccccttccgccccgccgggcgtcgcacgaggc

SEQ ID NO: 441 - RNA, 73 nt, Homo sapiens
  source 1..73: mol_type = transcribed RNA
ctggtgtttgaggcgatgtggggatgtagagacaacttcccagtctcatttcctcatcctgccaggccaccat

SEQ ID NO: 442 - RNA, 103 nt, Homo sapiens
  source 1..103: mol_type = transcribed RNA
gtgtcggctgtggcgtgactgtccctctgtgtcccccactaggcccactgctcagtggagcgtggaggacgaggaggaggccgtccacgagcaatgccagcat

SEQ ID NO: 443 - RNA, 80 nt, Homo sapiens
  source 1..80: mol_type = transcribed RNA
ggcgcgtcgcccccctcagtccaccagagcccggatacctcagaaattcggctctgggtctgtggggagcgaaatgcaac

SEQ ID NO: 444 - RNA, 119 nt, Homo sapiens
  source 1..119: mol_type = transcribed RNA
agcctgcgccggagccggggcctgagcccgggccgcgcaggccgtgaactcgtcgagctgcgcgtgcggccggtgctcaacctgccgggtcctggccccgcgctcccgcgcgccctgga

SEQ ID NO: 445 - RNA, 56 nt, Homo sapiens
  source 1..56: mol_type = transcribed RNA
gtaggcaggggctggggtttcaggttctcagtcagaaccttggcccctctccccag

SEQ ID NO: 446 - RNA, 73 nt, Homo sapiens
  source 1..73: mol_type = transcribed RNA
aggccaggtgggtatggaggagccctcatatggcagttggcgagggcccagtgagcccctctctgctctccag

SEQ ID NO: 447 - RNA, 78 nt, Homo sapiens
  source 1..78: mol_type = transcribed RNA
gtagctgaggggatggtagaccggtgacgtgcacttcatttacgatgtaggtcacccgtttgactatccaccagcgcc

SEQ ID NO: 448 - RNA, 87 nt, Homo sapiens
  source 1..87: mol_type = transcribed RNA
gtgtggccggcaggcgggtgggcgggggcggccggtgggaaccccgccccgccccgcgcccgcactcacccgcccgtctccccacag

SEQ ID NO: 449 - RNA, 63 nt, Homo sapiens
  source 1..63: mol_type = transcribed RNA
aatgggtgggtgctggtgggagccgtgccctggccactcattcggctctctccctcaccctag

SEQ ID NO: 450 - RNA, 73 nt, Homo sapiens
  source 1..73: mol_type = transcribed RNA
ggggaggtacctgggacaggaggaggaggcagccttgcctcagaaaccaaactgtcaaaagtgtaggttccac

SEQ ID NO: 451 - RNA, 62 nt, Homo sapiens
  source 1..62: mol_type = transcribed RNA
ttaccttgtggggttggagagctggctggtccagcccctcagaagctctcccctccccgcag

SEQ ID NO: 452 - RNA, 56 nt, Homo sapiens
  source 1..56: mol_type = transcribed RNA
cgctcgggcggaggtggttgagtgccgactggcgcctgacccaccccctcccgcag

SEQ ID NO: 453 - RNA, 81 nt, Homo sapiens
  source 1..81: mol_type = transcribed RNA
ctccctgggagggcgtggatgatggtgggagaggagccccactgtggaagtctgacccccacatcgccccaccttccccag

SEQ ID NO: 454 - RNA, 66 nt, Homo sapiens
  source 1..66: mol_type = transcribed RNA
cctgggaacgggttccggcagacgctgaggttgcgttgacgctcgcgccccggctcccgttccagg

SEQ ID NO: 455 - RNA, 61 nt, Homo sapiens
  source 1..61: mol_type = transcribed RNA
cctgcggggacaggccagggcatctaggctgtgcacagtgacgcccctcctgcccccacag

SEQ ID NO: 456 - RNA, 60 nt, Homo sapiens
  source 1..60: mol_type = transcribed RNA
ggcaggagggctgtgccaggttggctgggccaggcctgacctgccagcacctccctgcag

SEQ ID NO: 457 - RNA, 71 nt, Homo sapiens
  source 1..71: mol_type = transcribed RNA
gtgtctgtgccggtcccaggagaacctgcagaggcatcgggtcagcggtgctcctgcgggccgacactcac

SEQ ID NO: 458 - RNA, 65 nt, Homo sapiens
  source 1..65: mol_type = transcribed RNA
tgtctggggatttggagaagtggtgagcgcaggtctttggcaccatctcccctggtcccttggct

SEQ ID NO: 459 - RNA, 68 nt, Homo sapiens
  source 1..68: mol_type = transcribed RNA
agggttggggggacaggatgagaggctgtcttcattccctcttgaccacccctcgtttcttcccccag

SEQ ID NO: 460 - RNA, 87 nt, Homo sapiens
  source 1..87: mol_type = transcribed RNA
accgcagggaaaatgagggacttttgggggcagatgtgtttccattccactatcataatgcccctaaaaatccttattgctcttgca

SEQ ID NO: 461 - RNA, 124 nt, Homo sapiens
  source 1..124: mol_type = transcribed RNA
aaccctccttgggaagtgaagctcaggctgtgatttcaagccagggggcgtttttctataactggatgaaaagcacctccagagcttgaagctcacagtttgagagcaatcgtctaaggaagtt

SEQ ID NO: 462 - RNA, 72 nt, Homo sapiens
  source 1..72: mol_type = transcribed RNA
cagccaggagggaaggggctgagaacaggacctgtgctcactggggcctgcatgacccttccctccccacag

SEQ ID NO: 463 - RNA, 61 nt, Homo sapiens
  source 1..61: mol_type = transcribed RNA
gccctccgcccgtgcaccccggggcaggagaccccgcgggacgcgccgaggtaggggggac

SEQ ID NO: 464 - RNA, 67 nt, Homo sapiens
  source 1..67: mol_type = transcribed RNA
cagggaggaggtggtactaggggccagcaacctgattacccctctttggccctttgtacccctccag

SEQ ID NO: 465 - RNA, 136 nt, Homo sapiens
  source 1..136: mol_type = transcribed RNA
ccgcttgcctcgcccagcgcagccccggccgctgggcgcacccgtcccgttcgtccccggacgttgctctctaccccgggaacgtcgagactggagcgcccgaactgagccaccttcgcggaccccgagagcggcg

SEQ ID NO: 466 - RNA, 115 nt, Homo sapiens
  source 1..115: mol_type = transcribed RNA
tctgaggagacctgggctgtcagaggccagggaaggggacgagggttggggaacaggtggttagcacttcatcctcgtctccctcccaggttagaagggcccccctctctgaagg

SEQ ID NO: 467 - RNA, 71 nt, Homo sapiens
  source 1..71: mol_type = transcribed RNA
tgacgggcgagcttttggcccgggttatacctgatgctcacgtataagacgagcaaaaagcttgttggtca

SEQ ID NO: 468 - RNA, 65 nt, Homo sapiens
  source 1..65: mol_type = transcribed RNA
agccctgggggtggtctctagccaaggctctggggtctcacccttggctggtctctgctccgcag

SEQ ID NO: 469 - RNA, 84 nt, Homo sapiens
  source 1..84: mol_type = transcribed RNA
ctccccatggccctgtctcccaacccttgtaccagtgctgggctcagaccctggtacaggcctgggggacagggacctggggac

SEQ ID NO: 470 - RNA, 60 nt, Homo sapiens
  source 1..60: mol_type = transcribed RNA
tgtgaatgacccccttccagagccaaaatcaccagggatggaggaggggtcttgggtact

SEQ ID NO: 471 - RNA, 68 nt, Homo sapiens
  source 1..68: mol_type = transcribed RNA
tgcccaggctggagcgagtgcagtggtgcagtcagtcctagctcactgcagcctcgaactcctgggct

SEQ ID NO: 472 - RNA, 63 nt, Homo sapiens
  source 1..63: mol_type = transcribed RNA
gagtcgaggactggtggaagggcctttcccctcagaccaaggccctggccccagcttcttctc

SEQ ID NO: 473 - RNA, 74 nt, Homo sapiens
  source 1..74: mol_type = transcribed RNA
gcagcccggtgagcgctcgctggcctggcagtgcgtcggaagaacagggcgggtggggccgcgcacatctctgc

SEQ ID NO: 474 - RNA, 54 nt, Homo sapiens
  source 1..54: mol_type = transcribed RNA
agcagcaggggagagagaggagtcctctagacaccgactctgtctcctgcagat

SEQ ID NO: 475 - RNA, 83 nt, Homo sapiens
  source 1..83: mol_type = transcribed RNA
gctgctgttgggagaccctggtctgcactctatctgtattcttactgaagggagtgcagggcagggtttcccatacagagggc

SEQ ID NO: 476 - RNA, 83 nt, Homo sapiens
  source 1..83: mol_type = transcribed RNA
acggcatctttgcactcagcaggcaggctggtgcagcccgtggtgggggaccatcctgcctgctgtggggtaaggacggctgt

SEQ ID NO: 477 - RNA, 118 nt, Homo sapiens
  source 1..118: mol_type = transcribed RNA
gcaggtgaactggcaggccaggaagaggaggaagccctggaggggctggaggtgatggatgttttcctccggttctcagggctccacctctttcgggccgtagagccagggctggtgc

SEQ ID NO: 478 - RNA, 66 nt, Homo sapiens
  source 1..66: mol_type = transcribed RNA
tgactggggagcagaaggagaacccaagaaaagctgacttggaggtccctccttctgtccccacag

SEQ ID NO: 479 - RNA, 49 nt, Homo sapiens
  source 1..49: mol_type = transcribed RNA
gtgaggctggagtgagcggagatcgtaccactgcactccaacctggtga

SEQ ID NO: 480 - RNA, 84 nt, Homo sapiens
  source 1..84: mol_type = transcribed RNA
ccgcagccgccgcgccgggcccgggttggccgctgacccccgcggggcccccggcggccggggcgggggcgggggctgccccgg

SEQ ID NO: 481 - RNA, 113 nt, Homo sapiens
  source 1..113: mol_type = transcribed RNA
agcatgacagaggagaggtggaggtaggcgagagtaatataatttctccaggagaacatctgagaggggaagttgctttcctgccctggccctttcaccctcctgagtttggg

SEQ ID NO: 482 - RNA, 62 nt, Homo sapiens
  source 1..62: mol_type = transcribed RNA
ccttctgcggcagagctggggtcaccagccctcatgtacttgtgacttctcccctgccacag

SEQ ID NO: 483 - RNA, 54 nt, Homo sapiens
  source 1..54: mol_type = transcribed RNA
ggacaagggcggcgcgaccggcccggggctcttgggcggccgcgtttcccctcc

SEQ ID NO: 484 - RNA, 109 nt, Homo sapiens
  source 1..109: mol_type = transcribed RNA
ggtcgggctcaccatgacacagtgtgagacctcgggctacaacacaggacccgggcgctgctctgacccctcgtgtcttgtgttgcagccggagggacgcaggtccgca

SEQ ID NO: 485 - RNA, 81 nt, Homo sapiens
  source 1..81: mol_type = transcribed RNA
tccactgctgccgccgtcgccgccacccgagccggagcgggctgggccgccaaggcaagatggtggactacagcgtgtggg

SEQ ID NO: 486 - RNA, 69 nt, Homo sapiens
  source 1..69: mol_type = transcribed RNA
tggggtaggggtgggggaattcaggggtgtcgaactcatggctgccacctttgtgtccccatcctgcag

SEQ ID NO: 487 - RNA, 120 nt, Homo sapiens
  source 1..120: mol_type = transcribed RNA
gatccagggaaccctagagcagggggatggcagagcaaaattcatggcctacagctgcctcttgccaaactgcactggattttgtgtctcccattccccagagctgtctgaggtgctttg

SEQ ID NO: 488 - RNA, 60 nt, Homo sapiens
  source 1..60: mol_type = transcribed RNA
caaggtgggggagatgggggttgaacttcatttctcatgctcatccccatctcctttcag

SEQ ID NO: 489 - RNA, 85 nt, Homo sapiens
  source 1..85: mol_type = transcribed RNA
ggggaggtagggaaaaggaagggggaggagaaggtgagaccaatgtcctgggtgccactcctgcccagtgcctcccttcctcgtt

SEQ ID NO: 490 - RNA, 85 nt, Homo sapiens
  source 1..85: mol_type = transcribed RNA
gggacggggcctgcaggcagaagtggggctgacagggcagagggttgcgccccctcaccaccccttctgcctgcagcggtgggct

SEQ ID NO: 491 - RNA, 71 nt, Homo sapiens
  source 1..71: mol_type = transcribed RNA
ggggcctgcaggcagaagtggggctgacagggcagagggttgcgccccctcaccaccccttctgcctgcag

SEQ ID NO: 492 - RNA, 97 nt, Homo sapiens
  source 1..97: mol_type = transcribed RNA
ggtgggaggattgcttgagcctggaagctggagcctgcagtgaactatcattgtgccactgtactccagcctaggcaacaaaatgaaatcctgtcta

SEQ ID NO: 493 - RNA, 63 nt, Homo sapiens
  source 1..63: mol_type = transcribed RNA
ctgagcctggaagctggagcctgcagtgagctatgatcatgtccctgtactctagcctgggca

SEQ ID NO: 494 - RNA, 55 nt, Homo sapiens
  source 1..55: mol_type = transcribed RNA
aatctgccagcttccacagtggcagattttcccatagtgggaagctggcagattc

SEQ ID NO: 495 - RNA, 101 nt, Homo sapiens
  source 1..101: mol_type = transcribed RNA
gatgggccccttgtgtcctgaattgggtgggggctctgagtggggaaagtgggggcctaggggaggtcacagttgggtctaggggtcaggagggcccagga

SEQ ID NO: 496 - RNA, 74 nt, Homo sapiens
  source 1..74: mol_type = transcribed RNA
gctctagcctaattttagatctggtctgcttcagtttcactccaagcagacttgacctacaattagcctagagc

SEQ ID NO: 497 - RNA, 74 nt, Homo sapiens; identical to SEQ ID NO: 496
  source 1..74: mol_type = transcribed RNA

SEQ ID NO: 498 - RNA, 83 nt, Homo sapiens
  source 1..83: mol_type = transcribed RNA
aatagagggtgcacaggcacgggagctcaggtgaggcagggagctgagctcacctgacctcccatgcctgtgcaccctctatt

SEQ ID NO: 499 - RNA, 71 nt, Homo sapiens
  source 1..71: mol_type = transcribed RNA
gcagaggtgagttgacctccacagggccacccagggagtaagtagccaagtggaagttactttacctctgt

SEQ ID NO: 500 - RNA, 63 nt, Homo sapiens
  source 1..63: mol_type = transcribed RNA
gccaaggactgatcctctcgggcagggagtcagaggggaccgcccgagaggatccgtccctgc

SEQ ID NO: 501 - RNA, 66 nt, Homo sapiens
  source 1..66: mol_type = transcribed RNA
tcaagacggggagtcaggcagtggtggagatggagagccctgagcctccactctcctggcccccag

SEQ ID NO: 502 - RNA, 68 nt, Homo sapiens
  source 1..68: mol_type = transcribed RNA
gggcgcagggggactgggggtgagcaggcccagaacccagctcgtgctcactctcagtccctccctag

SEQ ID NO: 503 - RNA, 71 nt, Homo sapiens
  source 1..71: mol_type = transcribed RNA
gggtaaaggggcagggacgggtggccccaggaagaagggcctggtggagccgctcttctccctgcccacag

SEQ ID NO: 504 - RNA, 60 nt, Homo sapiens
  source 1..60: mol_type = transcribed RNA
ggtgcctcgggagggcatgggccaggccacataatgagccaaacccctgtctacccgcag

SEQ ID NO: 505 - RNA, 61 nt, Homo sapiens
  source 1..61: mol_type = transcribed RNA
tcacccggtgagggcgggtggaggaggagggtccccaccatcagccttcactgggacggga

SEQ ID NO: 506 - RNA, 82 nt, Homo sapiens
  source 1..82: mol_type = transcribed RNA
ctgacttttttagggagtagaagggtggggagcatgaacaatgtttctcactccctacccctccactccccaaaaaagtcag

SEQ ID NO: 507 - RNA, 67 nt, Homo sapiens
  source 1..67: mol_type = transcribed RNA
aaatctctctccatatctttcctgcagcccccaggtgggggggaagaaaaggtggggaattagattc

SEQ ID NO: 508 - RNA, 88 nt, Homo sapiens
  source 1..88: mol_type = transcribed RNA
gtggggccaggcggtggtgggcactgctggggtgggcacagcagccatgcagagcgggcatttgaccccgtgccacccttttccccag

SEQ ID NO: 509 - RNA, 86 nt, Homo sapiens
  source 1..86: mol_type = transcribed RNA
ggcttagaaacagtccctaggtaggatttggggaggagctaagaagcccctacagggcccagaggtggggactgagccttagttgg

SEQ ID NO: 510 - RNA, 70 nt, Homo sapiens
  source 1..70: mol_type = transcribed RNA
acaaatagcttcagggagtcaggggagggcagaaatagatggccttcccctgctgggaagaaagtgggtc

SEQ ID NO: 511 - RNA, 86 nt, Homo sapiens
  source 1..86: mol_type = transcribed RNA
agggagaagggtcggggcagggagggcagggcaggctctggggtggggggtctgtgagtcagccacggctctgcccacgtctcccc

SEQ ID NO: 512 - RNA, 73 nt, Homo sapiens
  source 1..73: mol_type = transcribed RNA
cggcgacggcggggtgggtgaggtcgggccccaagactcggggtttgccgggcgcctcagttcaccgcggccg

SEQ ID NO: 513 - RNA, 90 nt, Homo sapiens
  source 1..90: mol_type = transcribed RNA
gtgtctctctggagaccctgcagccttcccacccaccagggagctttccatgggctgtggggaaggcgtcagtgtcgggtgagggaacac

SEQ ID NO: 514 - RNA, 96 nt, Homo sapiens
  source 1..96: mol_type = transcribed RNA
gctctggggcgtgccgccgccgtcgctgccacctcccctaccgctagtggaagaagatggcggaaggcggagcggcggatctggacacccagcggt

SEQ ID NO: 515 - RNA, 60 nt, Homo sapiens
  source 1..60: mol_type = transcribed RNA
aggcctagggggtggcaggctggccatcagtgtgggctaaccctgtcctctccctcccag

SEQ ID NO: 516 - RNA, 67 nt, Homo sapiens
  source 1..67: mol_type = transcribed RNA
ccagacccctggggctgggcaggcggaaagaggtctgaactgcctctgcctccttggtctccggcag

SEQ ID NO: 517 - RNA, 61 nt, Homo sapiens
  source 1..61: mol_type = transcribed RNA
gagggttggggtggagggccaaggagctgggtggggtgccaagcctctgtccccaccccag

SEQ ID NO: 518 - RNA, 93 nt, Homo sapiens
  source 1..93: mol_type = transcribed RNA
gtaaggagggggatgaggggtcatatctcttctcagggaaagcaggagcccttcagcagggtcagggcccctcatcttcccctcctttcccag

SEQ ID NO: 519 - RNA, 65 nt, Homo sapiens
  source 1..65: mol_type = transcribed RNA
gtctcctggggggaggagaccctgctctccctggcagcaagcctctcctgcccttccagattagc

SEQ ID NO: 520 - RNA, 82 nt, Homo sapiens
  source 1..82: mol_type = transcribed RNA
gcttcgctcccctccgccttctcttcccggttcttcccggagtcgggaaaagctgggttgagagggcgaaaaaggatgaggt

SEQ ID NO: 521 - RNA, 93 nt, Homo sapiens
  source 1..93: mol_type = transcribed RNA
ccttccggcgtcccaggcggggcgccgcgggaccgccctcgtgtctgtggcggtgggatcccgcggccgtgttttcctggtggcccggccatg

SEQ ID NO: 522 - RNA, 75 nt, Homo sapiens
  source 1..75: mol_type = transcribed RNA
tggagtgggggggcaggaggggctcagggagaaagtgcatacagcccctggccctctctgcccttccgtcccctg

SEQ ID NO: 523 - RNA, 77 nt, Homo sapiens
  source 1..77: mol_type = transcribed RNA
gagttgggaggttccctctccaaatgtgtcttgatcccccaccccaagacacatttggagagggaccctcccaactc

SEQ ID NO: 524 - RNA, 84 nt, Homo sapiens
  source 1..84: mol_type = transcribed RNA
tgtgcagtgggaaggggggccgatacactgtacgagagtgagtagcaggtctcacagtgaaccggtctctttccctactgtgtc

SEQ ID NO: 525 - RNA, 86 nt, Homo sapiens
  source 1..86: mol_type = transcribed RNA
tgccagtctctaggtccctgagaccctttaacctgtgaggacatccagggtcacaggtgaggttcttgggagcctggcgtctggcc

SEQ ID NO: 526 - RNA, 92 nt, Homo sapiens
  source 1..92: mol_type = transcribed RNA
cggctggacagcgggcaacggaatcccaaaagcagctgttgtctccagagcattccagctgcgcttggatttcgtcccctgctctcctgcct

SEQ ID NO: 527 - RNA, 96 nt, Homo sapiens
  source 1..96: mol_type = transcribed RNA
cgggccccgggcgggcgggagggacgggacgcggtgcagtgttgttttttcccccgccaatattgcactcgtcccggcctccggcccccccggccc

SEQ ID NO: 528 - RNA, 73 nt, Homo sapiens
  source 1..73: mol_type = transcribed RNA
tgtatccttgaatggatttttggagcaggagtggacacctgacccaaaggaaatcaatccataggctagcaat

SEQ ID NO: 529 - RNA, 75 nt, Homo sapiens
  source 1..75: mol_type = transcribed RNA
tcatccctgggtggggatttgttgcattacttgtgttctatataaagtattgcacttgtcccggcctgtggaaga

SEQ ID NO: 530 - RNA, 82 nt, Homo sapiens
  source 1..82: mol_type = transcribed RNA
tgagctgttggattcggggccgtagcactgtctgagaggtttacatttctcacagtgaaccggtctctttttcagctgcttc

SEQ ID NO: 531 - RNA, 78 nt, Homo sapiens
  source 1..78: mol_type = transcribed RNA
gagcgtcacgttgacactcaaaaagtttcagattttggaacatttcggattttggatttttggatcagggatgctcaa

SEQ ID NO: 532 - RNA, 110 nt, Homo sapiens
  source 1..110: mol_type = transcribed RNA
tcacctggccatgtgacttgtgggcttccctttgtcatccttcgcctagggctctgagcagggcagggacagcaaaggggtgctcagttgtcacttcccacagcacggag

SEQ ID NO: 533 - RNA, 98 nt, Homo sapiens
  source 1..98: mol_type = transcribed RNA
ttgggcaaggtgcggggctagggctaacagcagtcttactgaaggtttcctggaaaccacgcacatgctgttgccactaacctcaaccttactcggtc

SEQ ID NO: 534 - RNA, 90 nt, Homo sapiens
  source 1..90: mol_type = transcribed RNA
aggcctcgctgttctctatggctttttattcctatgtgattctactgctcactcatatagggattggagccgtggcgcacggcggggaca

SEQ ID NO: 535 - RNA, 72 nt, Homo sapiens
  source 1..72: mol_type = transcribed RNA
cttgggaatggcaaggaaaccgttaccattactgagtttagtaatggtaatggttctcttgctatacccaga

SEQ ID NO: 536 - RNA, 85 nt, Homo sapiens
  source 1..85: mol_type = transcribed RNA
agggtagagggatgagggggaaagttctatagtcctgtaattagatctcaggactatagaactttccccctcatccctctgccct

SEQ ID NO: 537 - RNA, 78 nt, Homo sapiens
  source 1..78: mol_type = transcribed RNA
ctttctacacaggttgggatcggttgcaatgctgtgtttctgtatggtattgcacttgtcccggcctgttgagtttgg

SEQ ID NO: 538 - RNA, 90 nt, Homo sapiens
  source 1..90: mol_type = transcribed RNA
gagagaagcactggacttagggtcagaaggcctgagtctctctgctgcagatgggctctctgtccctgagccaagctttgtcctccctgg

SEQ ID NO: 539 - RNA, 97 nt, Homo sapiens
  source 1..97: mol_type = transcribed RNA
atctgagttgggagggtccctctccaaatgtgtcttggggtgggggatcaagacacatttggagagggaacctcccaactcggcctctgccatcatt

SEQ ID NO: 540 - RNA, 76 nt, Homo sapiens
  source 1..76: mol_type = transcribed RNA
gagggggaagacgggaggaaagaagggagtggttccatcacgcctcctcactcctctcctcccgtcttctcctctc

SEQ ID NO: 541 - RNA, 98 nt, Homo sapiens
  source 1..98: mol_type = transcribed RNA
acgaatggctatgcactgcacaaccctaggagagggtgccattcacatagactataattgaatggcgccactagggttgtgcagtgcacaacctacac

SEQ ID NO: 542 - RNA, 68 nt, Homo sapiens
  source 1..68: mol_type = transcribed RNA
ctccggtgcctactgagctgatatcagttctcattttacacactggctcagttcagcaggaacaggag

SEQ ID NO: 543 - RNA, 73 nt, Homo sapiens
  source 1..73: mol_type = transcribed RNA
ctctgcctcccgtgcctactgagctgaaacacagttggtttgtgtacactggctcagttcagcaggaacaggg

SEQ ID NO: 544 - RNA, 97 nt, Homo sapiens
  source 1..97: mol_type = transcribed RNA
ctcaggtgctctggctgcttgggttcctggcatgctgatttgtgacttaagattaaaatcacattgccagggattaccacgcaaccacgaccttggc

SEQ ID NO: 545 - RNA, 73 nt, Homo sapiens
  source 1..73: mol_type = transcribed RNA
ggccggctggggttcctggggatgggatttgcttcctgtcacaaatcacattgccagggatttccaaccgacc

SEQ ID NO: 546 - RNA, 85 nt, Homo sapiens
  source 1..85: mol_type = transcribed RNA
ggctgagccgcagtagttcttcagtggcaagctttatgtcctgacccagctaaagctgccagttgaagaactgttgccctctgcc

SEQ ID NO: 547 - RNA, 22 nt, Homo sapiens
  source 1..22: mol_type = transcribed RNA
ccccggtgttggggcgcgtctg

SEQ ID NO: 548 - RNA, 21 nt, Homo sapiens
  source 1..21: mol_type = transcribed RNA
cccggtgttggggcgcgtctg

SEQ ID NO: 549 - RNA, 22 nt, Homo sapiens
  source 1..22: mol_type = transcribed RNA
gtgggcgggggcaggtgtgtgg

SEQ ID NO: 550 - RNA, 15 nt, Homo sapiens
  source 1..15: mol_type = transcribed RNA
cgggggcaggtgtgt

SEQ ID NO: 551 - RNA, 20 nt, Homo sapiens
  source 1..20: mol_type = transcribed RNA
ccccggggagcccggcggtg

SEQ ID NO: 552 - RNA, 15 nt, Homo sapiens
  source 1..15: mol_type = transcribed RNA
accccggggagcccg

SEQ ID NO: 553 - RNA, 19 nt, Homo sapiens
  source 1..19: mol_type = transcribed RNA
gtgggttggggcgggctct

SEQ ID NO: 554 - RNA, 25 nt, Homo sapiens
  source 1..25: mol_type = transcribed RNA
actcggcgtggcgtcggtcgtggta

SEQ ID NO: 555 - RNA, 15 nt, Homo sapiens
  source 1..15: mol_type = transcribed RNA
actcggcgtggcgtc

SEQ ID NO: 556 - RNA, 19 nt, Homo sapiens
  source 1..19: mol_type = transcribed RNA
agtgggaggccagggcacg

SEQ ID NO: 557 - RNA, 15 nt, Homo sapiens
  source 1..15: mol_type = transcribed RNA
agggggagctgcagg

SEQ ID NO: 558 - RNA, 15 nt, Homo sapiens
  source 1..15: mol_type = transcribed RNA
tgaagcgggggggcg

SEQ ID NO: 559 - RNA, 21 nt, Homo sapiens
  source 1..21: mol_type = transcribed RNA
atccagttctctgagggggct

SEQ ID NO: 560 - RNA, 24 nt, Homo sapiens
  source 1..24: mol_type = transcribed RNA
gaaaagctgggttgagagggcaaa

SEQ ID NO: 561 - RNA, 15 nt, Homo sapiens
  source 1..15: mol_type = transcribed RNA
gaaaagctgggttga

SEQ ID NO: 562 - RNA, 15 nt, Homo sapiens
  source 1..15: mol_type = transcribed RNA
tctgggcgaggggtg

SEQ ID NO: 563 - RNA, 23 nt, Homo sapiens
  source 1..23: mol_type = transcribed RNA
ctcctggggcccgcactctcgct

SEQ ID NO: 564 - RNA, 18 nt, Homo sapiens
  source 1..18: mol_type = transcribed RNA
ctcctggggcccgcactc

SEQ ID NO: 565 - RNA, 22 nt, Homo sapiens
  source 1..22: mol_type = transcribed RNA
tggcggagcccattccatgcca

SEQ ID NO: 566 - RNA, 21 nt, Homo sapiens
  source 1..21: mol_type = transcribed RNA
ctggcggagcccattccatgc

SEQ ID NO: 567 - RNA, 17 nt, Homo sapiens
  source 1..17: mol_type = transcribed RNA
gatcccagcggtgcctc

SEQ ID NO: 568 - RNA, 15 nt, Homo sapiens
  source 1..15: mol_type = transcribed RNA
gatcccagcggtgcc

SEQ ID NO: 569 - RNA, 22 nt, Homo sapiens
  source 1..22: mol_type = transcribed RNA
actcaaaatgggggcgctttcc

SEQ ID NO: 570 - RNA, 21 nt, Homo sapiens
  source 1..21: mol_type = transcribed RNA
gccgggcgtggtggtgggggc

SEQ ID NO: 571 - RNA, 15 nt, Homo sapiens
  source 1..15: mol_type = transcribed RNA
tagccgggcgtggtg

SEQ ID NO: 572 - RNA, 20 nt, Homo sapiens
  source 1..20: mol_type = transcribed RNA
atcccaccactgccaccatt

SEQ ID NO: 573 - RNA, 15 nt, Homo sapiens
  source 1..15: mol_type = transcribed RNA
atcccaccactgcca

SEQ ID NO: 574 - RNA, 23 nt, Homo sapiens
  source 1..23: mol_type = transcribed RNA
aggactggactcccggcagcccc

SEQ ID NO: 575 - RNA, 18 nt, Homo sapiens
  source 1..18: mol_type = transcribed RNA
gcgtggggagctggtcct

SEQ ID NO: 576 - RNA, 24 nt, Homo sapiens
  source 1..24: mol_type = transcribed RNA
gtgagtgggagccggtggggctgg

SEQ ID NO: 577 - RNA, 15 nt, Homo sapiens
  source 1..15: mol_type = transcribed RNA
ggggctggagtaagg

SEQ ID NO: 578 - RNA, 21 nt, Homo sapiens
  source 1..21: mol_type = transcribed RNA
cccagcaggacgggagcgcgg

SEQ ID NO: 579 - RNA, 16 nt, Homo sapiens
  source 1..16: mol_type = transcribed RNA
aagctgggtcaaggag

SEQ ID NO: 580 - RNA, 22 nt, Homo sapiens
  source 1..22: mol_type = transcribed RNA
aggcagcggggtgtagtggata

SEQ ID NO: 581 - RNA, 21 nt, Homo sapiens
  source 1..21: mol_type = transcribed RNA
aggcagcggggtgtagtggat

SEQ ID NO: 582 - RNA, 21 nt, Homo sapiens
  source 1..21: mol_type = transcribed RNA
cagcctgagtgacagagcaag

SEQ ID NO: 583 - RNA, 15 nt, Homo sapiens
  source 1..15: mol_type = transcribed RNA
actgcactccagcct

SEQ ID NO: 584 - RNA, 24 nt, Homo sapiens
  source 1..24: mol_type = transcribed RNA
gatgcgccgcccactgccccgcgc

SEQ ID NO: 585 - RNA, 15 nt, Homo sapiens
  source 1..15: mol_type = transcribed RNA
gcccactgccccgcg

SEQ ID NO: 586 - RNA, 21 nt, Homo sapiens
  source 1..21: mol_type = transcribed RNA
cggatccgagtcacggcacca

SEQ ID NO: 587 - RNA, 15 nt, Homo sapiens
  source 1..15: mol_type = transcribed RNA
ggatccgagtcacgg

SEQ ID NO: 588 - RNA, 25 nt, Homo sapiens
  source 1..25: mol_type = transcribed RNA
agcggggaggaagtgggcgctgctt

SEQ ID NO: 589 - RNA, 21 nt, Homo sapiens
  source 1..21: mol_type = transcribed RNA
agcggggaggaagtgggcgct

SEQ ID NO: 590 - RNA, 24 nt, Homo sapiens
  source 1..24: mol_type = transcribed RNA
acgcccttcccccccttcttcacc

SEQ ID NO: 591 - RNA, 17 nt, Homo sapiens
  source 1..17: mol_type = transcribed RNA
acgcccttccccccctt

SEQ ID NO: 592 - RNA, 27 nt, Homo sapiens
  source 1..27: mol_type = transcribed RNA
gtgaacgggcgccatcccgaggctttg

SEQ ID NO: 593 - RNA, 16 nt, Homo sapiens
  source 1..16: mol_type = transcribed RNA
gtgaacgggcgccatc

SEQ ID NO: 594 - RNA, 23 nt, Homo sapiens
  source 1..23: mol_type = transcribed RNA
ggcccggccgtgcctgaggtttc

SEQ ID NO: 595 - RNA, 15 nt, Homo sapiens
  source 1..15: mol_type = transcribed RNA
tgtggcggtgggatc

SEQ ID NO: 596 - RNA, 19 nt, Homo sapiens
  source 1..19: mol_type = transcribed RNA
gcaggcgaggctgggctga

SEQ ID NO: 597 - RNA, 16 nt, Homo sapiens
  source 1..16: mol_type = transcribed RNA
aggcgaggctgggctg

SEQ ID NO: 598 - RNA, 18 nt, Homo sapiens
  source 1..18: mol_type = transcribed RNA
gggtgagggcaggtggtt

SEQ ID NO: 599 - RNA, 15 nt, Homo sapiens
  source 1..15: mol_type = transcribed RNA
gggtgagggcaggtg

SEQ ID NO: 600 - RNA, 22 nt, Homo sapiens
  source 1..22: mol_type = transcribed RNA
actcaaactgtgggggcacttt

SEQ ID NO: 601 - RNA, 19 nt, Homo sapiens
  source 1..19: mol_type = transcribed RNA
actcaaactgtgggggcac

SEQ ID NO: 602 - RNA, 22 nt, Homo sapiens
  source 1..22: mol_type = transcribed RNA
gagggttgggtggaggctctcc

SEQ ID NO: 603 - RNA, 15 nt, Homo sapiens
  source 1..15: mol_type = transcribed RNA
gagggttgggtggag

SEQ ID NO: 604 - RNA, 21 nt, Homo sapiens
  source 1..21: mol_type = transcribed RNA
caactctgatctcttcatcta

SEQ ID NO: 605 - RNA, 20 nt, Homo sapiens
  source 1..20: mol_type = transcribed RNA
tctcttcatctaccccccag

SEQ ID NO: 606 - RNA, 22 nt, Homo sapiens
  source 1..22: mol_type = transcribed RNA
cagccctcctcccgcacccaaa

SEQ ID NO: 607 - RNA, 21 nt, Homo sapiens
  source 1..21: mol_type = transcribed RNA
cagccctcctcccgcacccaa

SEQ ID NO: 608 - RNA, 23 nt, Homo sapiens
  source 1..23: mol_type = transcribed RNA
ggggggatgtgcatgctggttgg

SEQ ID NO: 609 - RNA, 15 nt, Homo sapiens
  source 1..15: mol_type = transcribed RNA
atcagcgtgcacttc

SEQ ID NO: 610 - RNA, 22 nt, Homo sapiens
  source 1..22: mol_type = transcribed RNA
tcctgcagagaggaagcccttc

SEQ ID NO: 611 - RNA, 18 nt, Homo sapiens
  source 1..18: mol_type = transcribed RNA
cctgcagagaggaagccc

SEQ ID NO: 612 - RNA, 17 nt, Homo sapiens
  source 1..17: mol_type = transcribed RNA
tgcaggggcaggccagc

SEQ ID NO: 613 - RNA, 15 nt, Homo sapiens
  source 1..15: mol_type = transcribed RNA
cctgttcttgccagg

SEQ ID NO: 614 - RNA, 21 nt, Homo sapiens
  source 1..21: mol_type = transcribed RNA
ttgaggagacatggtgggggc

SEQ ID NO: 615 - RNA, 15 nt, Homo sapiens
  source 1..15: mol_type = transcribed RNA
ttgaggagacatggt

SEQ ID NO: 616 - RNA, 24 nt, Homo sapiens
  source 1..24: mol_type = transcribed RNA
atatacagggggagactctcattt

SEQ ID NO: 617 - RNA, 15 nt, Homo sapiens
  source 1..15: mol_type = transcribed RNA
atatacagggggaga

SEQ ID NO: 618 - RNA, 24 nt, Homo sapiens
  source 1..24: mol_type = transcribed RNA
cgggcgtggtggtgggggtgggtg

SEQ ID NO: 619 - RNA, 15 nt, Homo sapiens
  source 1..15: mol_type = transcribed RNA
cgggcgtggtggtgg

SEQ ID NO: 620 - RNA, 22 nt, Homo sapiens
  source 1..22: mol_type = transcribed RNA
cctcacacctgcctcgcccccc

SEQ ID NO: 621 - RNA, 15 nt, Homo sapiens
  source 1..15: mol_type = transcribed RNA
tcacacctgcctcgc

SEQ ID NO: 622 - RNA, 23 nt, Homo sapiens
  source 1..23: mol_type = transcribed RNA
aatatacagggggagactcttat

SEQ ID NO: 623 - RNA, 25 nt, Homo sapiens
  source 1..25: mol_type = transcribed RNA
aaggcagggcccccgctccccgggc

SEQ ID NO: 624 - RNA, 15 nt, Homo sapiens
  source 1..15: mol_type = transcribed RNA
gtgtgttgaggaagg

SEQ ID NO: 625 - RNA, 24 nt, Homo sapiens
  source 1..24: mol_type = transcribed RNA
tggggagctgaggctctgggggtg

SEQ ID NO: 626 - RNA, 15 nt, Homo sapiens
  source 1..15: mol_type = transcribed RNA
ggccctggggagctg

SEQ ID NO: 627 - RNA, 23 nt, Homo sapiens
  source 1..23: mol_type = transcribed RNA
gtgggggagaggctgtcttgtgt

SEQ ID NO: 628 - RNA, 15 nt, Homo sapiens
  source 1..15: mol_type = transcribed RNA
gtgtgggggagaggc

SEQ ID NO: 629 - RNA, 26 nt, Homo sapiens
  source 1..26: mol_type = transcribed RNA
agggctggactcagcggcggagctgg

SEQ ID NO: 630 - RNA, 17 nt, Homo sapiens
  source 1..17: mol_type = transcribed RNA
gcggcggagctggctgc

SEQ ID NO: 631 - RNA, 21 nt, Homo sapiens
  source 1..21: mol_type = transcribed RNA
cttccccccagtaatcttcat

SEQ ID NO: 632 - RNA, 22 nt, Homo sapiens
  source 1..22: mol_type = transcribed RNA
accccactcctggtaccatagt

SEQ ID NO: 633 - RNA, 15 nt, Homo sapiens
  source 1..15: mol_type = transcribed RNA
accccactcctggta

SEQ ID NO: 634 - RNA, 20 nt, Homo sapiens
  source 1..20: mol_type = transcribed RNA
ggctccttggtctaggggta

SEQ ID NO: 635 - RNA, 15 nt, Homo sapiens
  source 1..15: mol_type = transcribed RNA
cttggtctaggggta

SEQ ID NO: 636 - RNA, 22 nt, Homo sapiens
  source 1..22: mol_type = transcribed RNA
gcacgactcagggcggagggaa

SEQ ID NO: 637 - RNA, 15 nt, Homo sapiens
  source 1..15: mol_type = transcribed RNA
agggcggagggaagt

SEQ ID NO: 638 - RNA, 26 nt, Homo sapiens
  source 1..26: mol_type = transcribed RNA
cgggtagagagggcagtgggaggtaa

SEQ ID NO: 639 - RNA, 15 nt, Homo sapiens
  source 1..15: mol_type = transcribed RNA
cgggtagagagggca

SEQ ID NO: 640 - RNA, 22 nt, Homo sapiens
  source 1..22: mol_type = transcribed RNA
tcccccggcctgctcatccccc

SEQ ID NO: 641 - RNA, 24 nt, Homo sapiens
  source 1..24: mol_type = transcribed RNA
cccggggcagattggtgtagggtg

SEQ ID NO: 642 - RNA, 17 nt, Homo sapiens
  source 1..17: mol_type = transcribed RNA
cggggcagattggtgta

SEQ ID NO: 643 - RNA, 17 nt, Homo sapiens
  source 1..17: mol_type = transcribed RNA
tcctagtcacggcacca

SEQ ID NO: 644 - RNA, 30 nt, Homo sapiens
  source 1..30: mol_type = transcribed RNA
gctgggctgggacggacacccggcctccac

SEQ ID NO: 645 - RNA, 19 nt, Homo sapiens
  source 1..19: mol_type = transcribed RNA
gaggctgggctgggacgga

SEQ ID NO: 646 - RNA, 22 nt, Homo sapiens
  source 1..22: mol_type = transcribed RNA
aggagcaaggcggcatctctct

SEQ ID NO: 647 - RNA, 18 nt, Homo sapiens
  source 1..18: mol_type = transcribed RNA
gagcaaggcggcatctct

SEQ ID NO: 648 - RNA, 20 nt, Homo sapiens
  source 1..20: mol_type = transcribed RNA
ggctggtcagatgggagtgg

SEQ ID NO: 649 - RNA, 24 nt, Homo sapiens
  source 1..24: mol_type = transcribed RNA
agcagggctggggattgcagtatc

SEQ ID NO: 650 - RNA, 19 nt, Homo sapiens
  source 1..19: mol_type = transcribed RNA
tgctgctcccagtcctgcc

SEQ ID NO: 651 - RNA, 25 nt, Homo sapiens
  source 1..25: mol_type = transcribed RNA
gctgcgggctgcggtcagggcgatc

SEQ ID NO: 652 - RNA, 20 nt, Homo sapiens
  source 1..20: mol_type = transcribed RNA
gctgcgggctgcggtcaggg

SEQ ID NO: 653 - RNA, 22 nt, Homo sapiens
  source 1..22: mol_type = transcribed RNA
agctctgctgctcactggcagt

SEQ ID NO: 654 - RNA, 20 nt, Homo sapiens
  source 1..20: mol_type = transcribed RNA
agctctgctgctcactggca

SEQ ID NO: 655 - RNA, 16 nt, Homo sapiens
  source 1..16: mol_type = transcribed RNA
ggcgcggagggcggac

SEQ ID NO: 656 - RNA, 15 nt, Homo sapiens
  source 1..15: mol_type = transcribed RNA
ggcgcggagggcgga

SEQ ID NO: 657 - RNA, 23 nt, Homo sapiens
  source 1..23: mol_type = transcribed RNA
agcagaggcagagaggctcaggg

SEQ ID NO: 658 - RNA, 15 nt, Homo sapiens
  source 1..15: mol_type = transcribed RNA
agcagaggcagagag

SEQ ID NO: 659 - RNA, 22 nt, Homo sapiens
  source 1..22: mol_type = transcribed RNA
tgggaggggagaggcagcaagc

SEQ ID NO: 660 - RNA, 16 nt, Homo sapiens
  source 1..16: mol_type = transcribed RNA
accagcctggggcatc

SEQ ID NO: 661 - RNA, 22 nt, Homo sapiens
  source 1..22: mol_type = transcribed RNA
aaaagctgggctgagaggcgac

SEQ ID NO: 662 - RNA, 15 nt, Homo sapiens
  source 1..15: mol_type = transcribed RNA
aaagctgggctgaga

SEQ ID NO: 663 - RNA, 25 nt, Homo sapiens
  source 1..25: mol_type = transcribed RNA
caggaaggatttagggacaggcttt

SEQ ID NO: 664 - RNA, 19 nt, Homo sapiens
  source 1..19: mol_type = transcribed RNA
caggaaggatttagggaca

SEQ ID NO: 665 - RNA, 23 nt, Homo sapiens
  source 1..23: mol_type = transcribed RNA
tggtgcggagagggcccacagtg

SEQ ID NO: 666 - RNA, 15 nt, Homo sapiens
  source 1..15: mol_type = transcribed RNA
gggtctggtgcggag

SEQ ID NO: 667 - RNA, 16 nt, Homo sapiens
  source 1..16: mol_type = transcribed RNA
ggacccagggagagac

SEQ ID NO: 668 - RNA, 22 nt, Homo sapiens
  source 1..22: mol_type = transcribed RNA
cgcggcggggacggcgattggt

SEQ ID NO: 669 - RNA, 17 nt, Homo sapiens
  source 1..17: mol_type = transcribed RNA
cggcggggacggcgatt

SEQ ID NO: 670 - RNA, 22 nt, Homo sapiens
  source 1..22: mol_type = transcribed RNA
ccggcagaggaggctgcagagg

SEQ ID NO: 671 - RNA, 19 nt, Homo sapiens
  source 1..19: mol_type = transcribed RNA
ccggcagaggaggctgcag

SEQ ID NO: 672 - RNA, 29 nt, Homo sapiens
  source 1..29: mol_type = transcribed RNA
gggagccgcggggatcgccgagggccggt

SEQ ID NO: 673 - RNA, 15 nt, Homo sapiens
  source 1..15: mol_type = transcribed RNA
ggcggcggtggtggg

SEQ ID NO: 674 - RNA, 23 nt, Homo sapiens
  source 1..23: mol_type = transcribed RNA
tgtagagcagggagcaggaagct

SEQ ID NO: 675 - RNA, 15 nt, Homo sapiens
  source 1..15: mol_type = transcribed RNA
cagggagcaggaagc

SEQ ID NO: 676 - RNA, 21 nt, Homo sapiens
  source 1..21: mol_type = transcribed RNA
ggcgggggtggcggcggcatc

SEQ ID NO: 677 - RNA, 15 nt, Homo sapiens
  source 1..15: mol_type = transcribed RNA
ggtggcggcggcatc

SEQ ID NO: 678 - RNA, 29 nt, Homo sapiens
  source 1..29: mol_type = transcribed RNA
ggtcaggcggctcggactgagcaggtggg

SEQ ID NO: 679 - RNA, 15 nt, Homo sapiens
  source 1..15: mol_type = transcribed RNA
agagtgtggtcaggc

SEQ ID NO: 680 - RNA, 28 nt, Homo sapiens
  source 1..28: mol_type = transcribed RNA
cagcctcccaaaatgctgggattacagg

SEQ ID NO: 681 - RNA, 15 nt, Homo sapiens
  source 1..15: mol_type = transcribed RNA
gcccacctcagcctc

SEQ ID NO: 682 - RNA, 28 nt, Homo sapiens
  source 1..28: mol_type = transcribed RNA
aggggtgctatctgtgattgagggacat

SEQ ID NO: 683 - RNA, 15 nt, Homo sapiens
  source 1..15: mol_type = transcribed RNA
gctatctgtgattga

SEQ ID NO: 684 - RNA, 19 nt, Homo sapiens
  source 1..19: mol_type = transcribed RNA
cgtcccaccccccactcct

SEQ ID NO: 685 - RNA, 23 nt, Homo sapiens
  source 1..23: mol_type = transcribed RNA
gccccggcgcgggcgggttctgg

SEQ ID NO: 686 - RNA, 16 nt, Homo sapiens
  source 1..16: mol_type = transcribed RNA
ggagccccggcgcggg

SEQ ID NO: 687 - RNA, 22 nt, Homo sapiens
  source 1..22: mol_type = transcribed RNA
accaggaggctgaggcccctca

SEQ ID NO: 688 - RNA, 15 nt, Homo sapiens
  source 1..15: mol_type = transcribed RNA
accaggaggctgagg

SEQ ID NO: 689 - RNA, 25 nt, Homo sapiens
  source 1..25: mol_type = transcribed RNA
tgcaggcagaagtggggctgacagg

SEQ ID NO: 690 - RNA, 20 nt, Homo sapiens
  source 1..20: mol_type = transcribed RNA
ctgcaggcagaagtggggct

SEQ ID NO: 691 - RNA, 22 nt, Homo sapiens
  source 1..22: mol_type = transcribed RNA
atggccagagctcacacagagg

SEQ ID NO: 692 - RNA, 21 nt, Homo sapiens
  source 1..21: mol_type = transcribed RNA
atggccagagctcacacagag

SEQ ID NO: 693 - RNA, 15 nt, Homo sapiens
  source 1..15: mol_type = transcribed RNA
ggcgcgcggcggctc

SEQ ID NO: 694 - RNA, 20 nt, Homo sapiens
  source 1..20: mol_type = transcribed RNA
atcccacctctgccaccaaa

SEQ ID NO: 695 - RNA, 15 nt, Homo sapiens
  source 1..15: mol_type = transcribed RNA
atcccacctctgcca

SEQ ID NO: 696 - RNA, 25 nt, Homo sapiens
  source 1..25: mol_type = transcribed RNA
tgggcaggggcttattgtaggagtc

SEQ ID NO: 697 - RNA, 19 nt, Homo sapiens
  source 1..19: mol_type = transcribed RNA
tgggcaggggcttattgta

SEQ ID NO: 698 - RNA, 23 nt, Homo sapiens
  source 1..23: mol_type = transcribed RNA
acaggagtgggggtgggacataa

SEQ ID NO: 699 - RNA, 20 nt, Homo sapiens
  source 1..20: mol_type = transcribed RNA
acaggagtgggggtgggaca

SEQ ID NO: 700 - RNA, 18 nt, Homo sapiens
  source 1..18: mol_type = transcribed RNA
gaggctgaaggaagatgg

SEQ ID NO: 701 - RNA, 15 nt, Homo sapiens
  source 1..15: mol_type = transcribed RNA
gaggctgaaggaaga

SEQ ID NO: 702 - RNA, 22 nt, Homo sapiens
  source 1..22: mol_type = transcribed RNA
ctgggctcgggacgcgcggctc

SEQ ID NO: 703 - RNA, 19 nt, Homo sapiens
  source 1..19: mol_type = transcribed RNA
ctgggctcgggacgcgcgg

SEQ ID NO: 704 - RNA, 18 nt, Homo sapiens
  source 1..18: mol_type = transcribed RNA
tctaggtggggagactga

SEQ ID NO: 705 - RNA, 16 nt, Homo sapiens
  source 1..16: mol_type = transcribed RNA
gtggggagactgacgg

SEQ ID NO: 706 - RNA, 15 nt, Homo sapiens
  source 1..15: mol_type = transcribed RNA
ggcggcgggcccggg

SEQ ID NO: 707 - RNA, 21 nt, Homo sapiens
  source 1..21: mol_type = transcribed RNA
gaggcgatgtggggatgtaga

SEQ ID NO: 708 - RNA, 20 nt, Homo sapiens
  source 1..20: mol_type = transcribed RNA
cccagtctcatttcctcatc

SEQ ID NO: 709 - RNA, 23 nt, Homo sapiens
  source 1..23: mol_type = transcribed RNA
tcggctctgggtctgtggggagc

SEQ ID NO: 710 - RNA, 15 nt, Homo sapiens
  source 1..15: mol_type = transcribed RNA
gcccggatacctcag

SEQ ID NO: 711 - RNA, 23 nt, Homo sapiens
  source 1..23: mol_type = transcribed RNA
gggatggtagaccggtgacgtgc

SEQ ID NO: 712 - RNA, 21 nt, Homo sapiens
  source 1..21: mol_type = transcribed RNA
taggtcacccgtttgactatc

SEQ ID NO: 713 - RNA, 15 nt, Homo sapiens
  source 1..15: mol_type = transcribed RNA
aggaggaggaggcag

SEQ ID NO: 714 - RNA, 24 nt, Homo sapiens
  source 1..24: mol_type = transcribed RNA
cgctcgggcggaggtggttgagtg

SEQ ID NO: 715 - RNA, 17 nt, Homo sapiens
  source 1..17: mol_type = transcribed RNA
tcgggcggaggtggttg

SEQ ID NO: 716 - RNA, 15 nt, Homo sapiens
  source 1..15: mol_type = transcribed RNA
acctctcctggcatc

SEQ ID NO: 717 - RNA, 23 nt, Homo sapiens
  source 1..23: mol_type = transcribed RNA
ggcaggagggctgtgccaggttg

SEQ ID NO: 718 - RNA, 17 nt, Homo sapiens
  source 1..17: mol_type = transcribed RNA
ggcaggagggctgtgcc

SEQ ID NO: 719 - RNA, 22 nt, Homo sapiens
  source 1..22: mol_type = transcribed RNA
tggggatttggagaagtggtga

SEQ ID NO: 720 - RNA, 27 nt, Homo sapiens
  source 1..27: mol_type = transcribed RNA
tgagggacttttgggggcagatgtgtt

SEQ ID NO: 721 - RNA, 17 nt, Homo sapiens
  source 1..17: mol_type = transcribed RNA
ggacttttgggggcaga

SEQ ID NO: 722 - RNA, 23 nt, Homo sapiens
  source 1..23: mol_type = transcribed RNA
tttcaagccagggggcgtttttc

SEQ ID NO: 723 - RNA, 18 nt, Homo sapiens
  source 1..18: mol_type = transcribed RNA
tttcaagccagggggcgt

SEQ ID NO: 724 - RNA, 15 nt, Homo sapiens
  source 1..15: mol_type = transcribed RNA
cccgcgggacgcgcc

SEQ ID NO: 725 - RNA, 22 nt, Homo sapiens
  source 1..22: mol_type = transcribed RNA
ccgggaacgtcgagactggagc

SEQ ID NO: 726 - RNA, 15 nt, Homo sapiens
  source 1..15: mol_type = transcribed RNA
cgggaacgtcgagac

SEQ ID NO: 727 - RNA, 25 nt, Homo sapiens
  source 1..25: mol_type = transcribed RNA
gggacgagggttggggaacaggtgg

SEQ ID NO: 728 - RNA, 15 nt, Homo sapiens
  source 1..15: mol_type = transcribed RNA
tggggaacaggtggt

SEQ ID NO: 729 - RNA, 24 nt, Homo sapiens
  source 1..24: mol_type = transcribed RNA
ctggtacaggcctgggggacaggg

SEQ ID NO: 730 - RNA, 18 nt, Homo sapiens
  source 1..18: mol_type = transcribed RNA
ctggtacaggcctggggg

SEQ ID NO: 731 - RNA, 21 nt, Homo sapiens
  source 1..21: mol_type = transcribed RNA
cccaggctggagcgagtgcag

SEQ ID NO: 732 - RNA, 15 nt, Homo sapiens
  source 1..15: mol_type = transcribed RNA
agctcactgcagcct

SEQ ID NO: 733 - RNA, 24 nt, Homo sapiens
  source 1..24: mol_type = transcribed RNA
tcgaggactggtggaagggccttt

SEQ ID NO: 734 - RNA, 16 nt, Homo sapiens
  source 1..16: mol_type = transcribed RNA
tcgaggactggtggaa

SEQ ID NO: 735 - RNA, 17 nt, Homo sapiens
  source 1..17: mol_type = transcribed RNA
ggtgagcgctcgctggc

SEQ ID NO: 736 - RNA, 15 nt, Homo sapiens
  source 1..15: mol_type = transcribed RNA
cggtgagcgctcgct

SEQ ID NO: 737 - RNA, 21 nt, Homo sapiens
  source 1..21: mol_type = transcribed RNA
cagcaggggagagagaggagt

SEQ ID NO: 738 - RNA, 20 nt, Homo sapiens
  source 1..20: mol_type = transcribed RNA
cagcaggggagagagaggag

SEQ ID NO: 739 - RNA, 22 nt, Homo sapiens
  source 1..22: mol_type = transcribed RNA
gggagtgcagggcagggtttcc

SEQ ID NO: 740 - RNA, 18 nt, Homo sapiens
  source 1..18: mol_type = transcribed RNA
agggagtgcagggcaggg

SEQ ID NO: 741 - RNA, 25 nt, Homo sapiens
  source 1..25: mol_type = transcribed RNA
aggaagccctggaggggctggaggt

SEQ ID NO: 742 - RNA, 15 nt, Homo sapiens
  source 1..15: mol_type = transcribed RNA
aggaagaggaggaag

SEQ ID NO: 743 - RNA, 24 nt, Homo sapiens
  source 1..24: mol_type = transcribed RNA
tgactggggagcagaaggagaacc

SEQ ID NO: 744 - RNA, 15 nt, Homo sapiens
  source 1..15: mol_type = transcribed RNA
gactggggagcagaa

SEQ ID NO: 745 - RNA, 19 nt, Homo sapiens
  source 1..19: mol_type = transcribed RNA
gaggctggagtgagcggag

SEQ ID NO: 746 - RNA, 15 nt, Homo sapiens
  source 1..15: mol_type = transcribed RNA
aggctggagtgagcg

SEQ ID NO: 747 - RNA, 17 nt, Homo sapiens
  source 1..17: mol_type = transcribed RNA
ggggcgggggcgggggc

SEQ ID NO: 748 - RNA, 15 nt, Homo sapiens
  source 1..15: mol_type = transcribed RNA
cgcgccgggcccggg

SEQ ID NO: 749 - RNA, 23 nt, Homo sapiens
  source 1..23: mol_type = transcribed RNA
tgaggatatggcagggaagggga

SEQ ID NO: 750 - RNA, 19 nt, Homo sapiens
  source 1..19: mol_type = transcribed RNA
tgaggatatggcagggaag

SEQ ID NO: 751 - RNA, 22 nt, Homo sapiens
  source 1..22: mol_type = transcribed RNA
attggagggtgtggaagacatc

SEQ ID NO: 752 - RNA, 16 nt, Homo sapiens
  source 1..16: mol_type = transcribed RNA
ttggagggtgtggaag

SEQ ID NO: 753 - RNA, 19 nt, Homo sapiens
  source 1..19: mol_type = transcribed RNA
cggcgcgaccggcccgggg

SEQ ID NO: 754 - RNA, 23 nt, Homo sapiens
  source 1..23: mol_type = transcribed RNA
ggctacaacacaggacccgggcg

SEQ ID NO: 755 - RNA, 21 nt, Homo sapiens
  source 1..21: mol_type = transcribed RNA
ggctacaacacaggacccggg

SEQ ID NO: 756 - RNA, 21 nt, Homo sapiens
  source 1..21: mol_type = transcribed RNA
ccggccgccggctccgccccg

SEQ ID NO: 757 - RNA, 17 nt, Homo sapiens
  source 1..17: mol_type = transcribed RNA
ccggccgccggctccgc

SEQ ID NO: 758 - RNA, 26 nt, Homo sapiens
  source 1..26: mol_type = transcribed RNA
caagatggtggactacagcgtgtggg

SEQ ID NO: 759 - RNA, 15 nt, Homo sapiens
  source 1..15: mol_type = transcribed RNA
aggcaagatggtgga

SEQ ID NO: 760 - RNA, 24 nt, Homo sapiens
  source 1..24: mol_type = transcribed RNA
cagggggatggcagagcaaaattc

SEQ ID NO: 761 - RNA, 17 nt, Homo sapiens
  source 1..17: mol_type = transcribed RNA
agggggatggcagagca

SEQ ID NO: 762 - RNA, 18 nt, Homo sapiens
  source 1..18: mol_type = transcribed RNA
aaaaggaagggggaggag

SEQ ID NO: 763 - RNA, 16 nt, Homo sapiens
  source 1..16: mol_type = transcribed RNA
aaggaagggggaggag

SEQ ID NO: 764 - RNA, 26 nt, Homo sapiens
  source 1..26: mol_type = transcribed RNA
aggagggaggagatgggccaagttcc

SEQ ID NO: 765 - RNA, 15 nt, Homo sapiens
  source 1..15: mol_type = transcribed RNA
gggaggagggaggag

SEQ ID NO: 766 - RNA, 25 nt, Homo sapiens
  source 1..25: mol_type = transcribed RNA
ctgcaggcagaagtggggctgacag

SEQ ID NO: 767 - RNA, 19 nt, Homo sapiens
  source 1..19: mol_type = transcribed RNA
caggcagaagtggggctga

SEQ ID NO: 768 - RNA, 27 nt, Homo sapiens
  source 1..27: mol_type = transcribed RNA
agcctggaagctggagcctgcagtgaa

SEQ ID NO: 769 - RNA, 15 nt, Homo sapiens
  source 1..15: mol_type = transcribed RNA
ggtgggaggattgct

SEQ ID NO: 770 - RNA, 22 nt, Homo sapiens
  source 1..22: mol_type = transcribed RNA
atagtgggaagctggcagattc

SEQ ID NO: 771 - RNA, 19 nt, Homo sapiens
  source 1..19: mol_type = transcribed RNA
atagtgggaagctggcaga

SEQ ID NO: 772 - RNA, 22 nt, Homo sapiens
  source 1..22: mol_type = transcribed RNA
taattttagatctggtctgctt

SEQ ID NO: 773 - RNA, 19 nt, Homo sapiens
  source 1..19: mol_type = transcribed RNA
aattttagatctggtctgc

SEQ ID NO: 774 - RNA, 22 nt, Homo sapiens
  source 1..22: mol_type = transcribed RNA
caggcacgggagctcaggtgag

SEQ ID NO: 775 - RNA, 17 nt, Homo sapiens
  source 1..17: mol_type = transcribed RNA
caggcacgggagctcag

SEQ ID NO: 776 - RNA, 20 nt, Homo sapiens
  source 1..20: mol_type = transcribed RNA
tggctgttggagggggcagg

SEQ ID NO: 777 - RNA, 15 nt, Homo sapiens
  source 1..15: mol_type = transcribed RNA
ggagggggcaggctc

SEQ ID NO: 778 - RNA, 19 nt, Homo sapiens
  source 1..19: mol_type = transcribed RNA
gagggcgggtggaggagga

SEQ ID NO: 779 - RNA, 15 nt, Homo sapiens
  source 1..15: mol_type = transcribed RNA
gcgggtggaggagga

SEQ ID NO: 780 - RNA, 23 nt, Homo sapiens
  source 1..23: mol_type = transcribed RNA
agggagtagaagggtggggagca

SEQ ID NO: 781 - RNA, 16 nt, Homo sapiens
  source 1..16: mol_type = transcribed RNA
tagggagtagaagggt

SEQ ID NO: 782 - RNA, 16 nt, Homo sapiens
  source 1..16: mol_type = transcribed RNA
tgggggggaagaaaag

SEQ ID NO: 783 - RNA, 23 nt, Homo sapiens
  source 1..23: mol_type = transcribed RNA
agggtcggggcagggagggcagg

SEQ ID NO: 784 - RNA, 15 nt, Homo sapiens
  source 1..15: mol_type = transcribed RNA
gggagaagggtcggg

SEQ ID NO: 785 - RNA, 23 nt, Homo sapiens
  source 1..23: mol_type = transcribed RNA
ggtgggtgaggtcgggccccaag

SEQ ID NO: 786 - RNA, 20 nt, Homo sapiens
  source 1..20: mol_type = transcribed RNA
cggggtgggtgaggtcgggc

SEQ ID NO: 787 - RNA, 23 nt, Homo sapiens
  source 1..23: mol_type = transcribed RNA
tggggaaggcgtcagtgtcgggt

SEQ ID NO: 788 - RNA, 16 nt, Homo sapiens
  source 1..16: mol_type = transcribed RNA
tggggaaggcgtcagt

SEQ ID NO: 789 - RNA, 23 nt, Homo sapiens
  source 1..23: mol_type = transcribed RNA
ctagtggaagaagatggcggaag

SEQ ID NO: 790 - RNA, 15 nt, Homo sapiens
  source 1..15: mol_type = transcribed RNA
tagtggaagaagatg

SEQ ID NO: 791 - RNA, 27 nt, Homo sapiens
  source 1..27: mol_type = transcribed RNA
gaaaagctgggttgagagggcgaaaaa

SEQ ID NO: 792 - RNA, 15 nt, Homo sapiens
  source 1..15: mol_type = transcribed RNA
cttctcttcccggtt

SEQ ID NO: 793 - RNA, 26 nt, Homo sapiens
  source 1..26: mol_type = transcribed RNA
cggtgggatcccgcggccgtgttttc

SEQ ID NO: 794 - RNA, 15 nt, Homo sapiens
  source 1..15: mol_type = transcribed RNA
ggggcgccgcgggac

SEQ ID NO: 795 - RNA, 22 nt, Homo sapiens
  source 1..22: mol_type = transcribed RNA
ggggggcaggaggggctcaggg

SEQ ID NO: 796 - RNA, 16 nt, Homo sapiens
  source 1..16: mol_type = transcribed RNA
gtgggggggcaggagg

SEQ ID NO: 797 - RNA, 20 nt, Homo sapiens
  source 1..20: mol_type = transcribed RNA
aagacacatttggagaggga

SEQ ID NO: 798 - RNA, 16 nt, Homo sapiens
  source 1..16: mol_type = transcribed RNA
agacacatttggagag

SEQ ID NO: 799 - RNA, 23 nt, Homo sapiens
  source 1..23: mol_type = transcribed RNA
gggggccgatacactgtacgaga

SEQ ID NO: 800 - RNA, 20 nt, Homo sapiens
  source 1..20: mol_type = transcribed RNA
gggggccgatacactgtacg

SEQ ID NO: 801 - RNA, 23 nt, Homo sapiens
  source 1..23: mol_type = transcribed RNA
cacaggtgaggttcttgggagcc

SEQ ID NO: 802 - RNA, 15 nt, Homo sapiens
  source 1..15: mol_type = transcribed RNA
acaggtgaggttctt

SEQ ID NO: 803 - RNA, 29 nt, Homo sapiens
  source 1..29: mol_type = transcribed RNA
caacggaatcccaaaagcagctgttgtct

SEQ ID NO: 804 - RNA, 15 nt, Homo sapiens
  source 1..15: mol_type = transcribed RNA
caacggaatcccaaa

SEQ ID NO: 805 - RNA, 26 nt, Homo sapiens
  source 1..26: mol_type = transcribed RNA
agggacgggacgcggtgcagtgttgt

SEQ ID NO: 806 - RNA, 15 nt, Homo sapiens
  source 1..15: mol_type = transcribed RNA
ggcgggcgggaggga

SEQ ID NO: 807 - RNA, 23 nt, Homo sapiens
  source 1..23: mol_type = transcribed RNA
gagggccccccctcaatcctgtt

SEQ ID NO: 808 - RNA, 15 nt, Homo sapiens
  source 1..15: mol_type = transcribed RNA
agggccccccctcaa

SEQ ID NO: 809 - RNA, 21 nt, Homo sapiens
  source 1..21: mol_type = transcribed RNA
gaatggatttttggagcagga

SEQ ID NO: 810 - RNA, 15 nt, Homo sapiens
  source 1..15: mol_type = transcribed RNA
gaatggatttttgga

SEQ ID NO: 811 - RNA, 25 nt, Homo sapiens
  source 1..25: mol_type = transcribed RNA
gggtggggatttgttgcattacttg

SEQ ID NO: 812 - RNA, 20 nt, Homo sapiens
  source 1..20: mol_type = transcribed RNA
gggtggggatttgttgcatt

SEQ ID NO: 813 - RNA, 23 nt, Homo sapiens
  source 1..23: mol_type = transcribed RNA
cggggccgtagcactgtctgaga

SEQ ID NO: 814 - RNA, 20 nt, Homo sapiens
  source 1..20: mol_type = transcribed RNA
cggggccgtagcactgtctg

SEQ ID NO: 815 - RNA, 20 nt, Homo sapiens
  source 1..20: mol_type = transcribed RNA
ggatttttggatcagggatg

SEQ ID NO: 816 - RNA, 15 nt, Homo sapiens
  source 1..15: mol_type = transcribed RNA
atttttggatcaggg

SEQ ID NO: 817 - RNA, 22 nt, Homo sapiens
  source 1..22: mol_type = transcribed RNA
ggcagggacagcaaaggggtgc

SEQ ID NO: 818 - RNA, 18 nt, Homo sapiens
  source 1..18: mol_type = transcribed RNA
gcagggacagcaaagggg

SEQ ID NO: 819 - RNA, 25 nt, Homo sapiens
  source 1..25: mol_type = transcribed RNA
tgcggggctagggctaacagcagtc

SEQ ID NO: 820 - RNA, 15 nt, Homo sapiens
  source 1..15: mol_type = transcribed RNA
tgcggggctagggct

SEQ ID NO: 821 - RNA, 22 nt, Homo sapiens
  source 1..22: mol_type = transcribed RNA
atatagggattggagccgtggc

SEQ ID NO: 822 - RNA, 20 nt, Homo sapiens
  source 1..20: mol_type = transcribed RNA
atatagggattggagccgtg

SEQ ID NO: 823 - RNA, 27 nt, Homo sapiens
  source 1..27: mol_type = transcribed RNA
aaaccgttaccattactgagtttagta

SEQ ID NO: 824 - RNA, 15 nt, Homo sapiens
  source 1..15: mol_type = transcribed RNA
gaaaccgttaccatt

SEQ ID NO: 825 - RNA, 26 nt, Homo sapiens
  source 1..26: mol_type = transcribed RNA
gactatagaactttccccctcatccc

SEQ ID NO: 826 - RNA, 15 nt, Homo sapiens
  source 1..15: mol_type = transcribed RNA
aactttccccctcat

SEQ ID NO: 827 - RNA, 28 nt, Homo sapiens
  source 1..28: mol_type = transcribed RNA
gtatggtattgcacttgtcccggcctgt

SEQ ID NO: 828 - RNA, 15 nt, Homo sapiens
  source 1..15: mol_type = transcribed RNA
tattgcacttgtccc

SEQ ID NO: 829 - RNA, 24 nt, Homo sapiens
  source 1..24: mol_type = transcribed RNA
agacacatttggagagggaacctc

SEQ ID NO: 830 - RNA, 24 nt, Homo sapiens
  source 1..24: mol_type = transcribed RNA
agacgggaggaaagaagggagtgg

SEQ ID NO: 831 - RNA, 15 nt, Homo sapiens
  source 1..15: mol_type = transcribed RNA
aagacgggaggaaag

SEQ ID NO: 832 - RNA, 26 nt, Homo sapiens
  source 1..26: mol_type = transcribed RNA
acaaccctaggagagggtgccattca

SEQ ID NO: 833 - RNA, 15 nt, Homo sapiens
  source 1..15: mol_type = transcribed RNA
acaaccctaggagag

SEQ ID NO: 834 - RNA, 24 nt, Homo sapiens
  source 1..24: mol_type = transcribed RNA
actggctcagttcagcaggaacag

SEQ ID NO: 835 - RNA, 15 nt, Homo sapiens
  source 1..15: mol_type = transcribed RNA
tggctcagttcagca

SEQ ID NO: 836 - RNA, 26 nt, Homo sapiens
  source 1..26: mol_type = transcribed RNA
aaaatcacattgccagggattaccac

SEQ ID NO: 837 - RNA, 15 nt, Homo sapiens
  source 1..15: mol_type = transcribed RNA
aatcacattgccagg

SEQ ID NO: 838 - RNA, 27 nt, Homo sapiens
  source 1..27: mol_type = transcribed RNA
atcacattgccagggatttccaaccga

SEQ ID NO: 839 - RNA, 24 nt, Homo sapiens
  source 1..24: mol_type = transcribed RNA
aatattgcactcgtcccggcctcc

SEQ ID NO: 840 - RNA, 15 nt, Homo sapiens
  source 1..15: mol_type = transcribed RNA
tattgcactcgtccc

SEQ ID NO: 841 - RNA, 25 nt, Homo sapiens
  source 1..25: mol_type = transcribed RNA
aagctgccagttgaagaactgttgc

SEQ ID NO: 842 - RNA, 15 nt, Homo sapiens
  source 1..15: mol_type = transcribed RNA
aagctgccagttgaa

(19) United States
(12) Patent Application Publication
(10) Pub. No.: US 2024/0425933 A1
(43) Pub. Date: Dec. 26, 2024

(54) KIT, DEVICE, AND METHOD FOR DETECTING OVARIAN TUMOR

(71) Applicants: TORAY INDUSTRIES, INC., Tokyo (JP); NATIONAL CANCER CENTER, Tokyo (JP)

(72) Inventors: Yuho KIDA, Kamakura-shi (JP); Satoko KOZONO, Kamakura-shi (JP); Hiroko SUDO, Kamakura-shi (JP); Satoshi KONDOU, Kamakura-shi (JP); Junpei KAWAUCHI, Kamakura-shi (JP); Hitoshi NOBUMASA, Kamakura-shi (JP); Takahiro OCHIYA, Tokyo (JP); Tomoyasu KATO, Tokyo (JP)

(73) Assignees: TORAY INDUSTRIES, INC., Tokyo (JP); NATIONAL CANCER CENTER, Tokyo (JP)

(21) Appl. No.: 18/821,252

(22) Filed: Aug. 30, 2024

Related U.S. Application Data
(62) Division of application No. 17/219,000, filed on Mar. 31, 2021, now Pat. No. 12,110,557.
Application No. 17/219,000 is a division of application No. 16/608,673, filed on Oct. 25, 2019, now Pat. No. 10,975,444.

(30) Foreign Application Priority Data
Apr. 28, 2017 (JP) 2017-090799

(51) Int. Cl. C12Q 1/6886; B01L 7/00; C12Q 1/6869

(52) U.S. Cl. CPC C12Q 1/6886; B01L 7/52; C12Q 1/6869; C12Q 2600/112; C12Q 2600/178; G01N 2800/7028

(57) ABSTRACT

This application provides a kit or a device for detection of ovarian tumor, comprising a nucleic acid(s) for detecting a miRNA(s) in a sample from a subject, and a method for detecting ovarian tumor, comprising measuring the miRNA(s) in vitro.

### CROSS-CITE TO RELATED APPLICATIONS

This application is a Divisional of copending U.S. application Ser. No. 17/219,000 filed Mar. 31, 2021, which is a Divisional of U.S. application Ser. No. 16/608,673, filed on Oct. 25, 2019 (now U.S. Pat. No. 10,975,444 issued Apr. 13, 2021), which is the National Phase under 35 U.S.C. § 371 of International Application No. PCT/JP2018/017125, filed on Apr. 27, 2018, which claims the benefit under 35 U.S.C. § 119(a) to Patent Application No. 2017-090799, filed in Japan on Apr. 28, 2017, all of which are hereby expressly incorporated by reference into the present application.

### REFERENCE TO ELECTRONIC SEQUENCE LISTING

The application contains a Sequence Listing which has been submitted electronically in .XML format and is hereby incorporated by reference in its entirety. Said .XML copy, created on Aug. 22, 2024, is named “PH-7323-PCT-US02.xml” and is 758,737 bytes in size. The sequence listing contained in this .XML file is part of the specification and is hereby incorporated by reference herein in its entirety.

### TECHNICAL FIELD

The present invention relates to a kit or a device for detection of ovarian tumor, comprising a nucleic acid capable of specifically binding to a particular miRNA or a complementary strand thereof, which is used for examining the presence or absence of ovarian tumor in a subject, and a method for detecting ovarian tumor, comprising measuring an expression level of the miRNA using the nucleic acid.

### BACKGROUND ART

The ovary is the female reproductive organ that produces an ovum and one each is found on both sides of the uterus. Fallopian tube is a tube through which an ovum, when released from the ovary, travels down into the uterus in which the fertilized ovum is implanted and grows into the fetus. The ovary further functions to secrete female hormones such as estrogen and progesterone. The ovary is considered an organ prone to develop tumors, which are roughly classified into surface epithelial-stromal tumors, sex cord-stromal tumor, and germ cell tumor based on the origin of tumorigenesis. The ovarian tumors are categorized into benign, borderline malignant, and malignant, and borderline malignant and malignant ovarian tumors are called ovarian cancers. Ovarian cancer incidence rate of 2012 in Japan disclosed by the Center for Cancer Control and Information Services, National Cancer Center was 9,384 people, in other words, 1 out of every 87 Japanese women had the cancer, causing the number of ovarian cancer deaths to climb to 4,840 people in 2014. In the United States, it is said that one in every 75 women develops ovarian cancer and the estimated number of people affected by ovarian cancer in 2017 climbed to 22,440 people, out of which about 14,080 people are expected to die.

A stage of progression of ovarian cancer and fallopian tube cancer is defined according to a tumor being unilateral or bilateral, the presence or absence of peritoneal dissemination, lymph node metastasis and distant metastasis, the size, and the like, and is classified into stages IA, IB, IC (IC1, IC2, IC3), IIA, IIB, IIIA1, IIIA2, IIIB, IIIC, IVA, and IVB. Five-year relative survival rate in ovarian cancer depends largely on a stage of progression of the cancer, and it is reported that the rate is 92% in the case of a localized cancer, 73% in the case of a peripheral region cancer, and 29% in the case of a distant metastatic cancer. Thus, early detection of ovarian cancer leads to improvement in survival rate and the provision of means for enabling the early detection is strongly required.

Treatment of ovarian cancer is surgical therapy in principal but drug therapy is concurrently used depending on a stage of progression, metastasis, systemic conditions, and the classification of ovarian cancer. Particularly, in the case that an ovarian cancer is detected early as stage I, a removal operation of an ovary or fallopian tube only on the side at which the cancer is detected may be sufficient instead of the total removal of the uterus, ovaries or fallopian tubes, whereby women can save a chance for future childbirth.

Early ovarian cancer and benign ovarian tumor are often asymptomatic, and even progressive ovarian cancer often only involves general symptoms which could be caused by other reasons such as abdominal bloating sensation and pains, because of which it is difficult to detect ovarian tumor based on subjective symptoms. Extensive studies on the screening test for ovarian cancer have been conducted but no substantial achievement is accomplished, and only two methods are actually usable: transvaginal ultrasound test and a CA-125 blood marker test. The secondary test for ovarian cancer includes imaging tests such as ultrasonography, CT scanning test, MRI, and PET/CT test.

As shown in Patent Literatures 1 to 4 and Non-Patent Literatures 1 to 3, there are reports, albeit at a research stage, on the detection of ovarian tumor using the expression levels of microRNAs (miRNAs) in biological samples including blood.

Specifically, Patent Literature 1 discloses a method for detecting ovarian cancer or predicting prognosis using miR-191, miR-24, miR-320, miR-328, miR-625-3p, miR-483-5p, miR-92a, miR-1290 and the like in plasma.
Patent Literature 2 discloses miR-135a-3 and the like in blood as biomarkers for gynecologic cancers.
Patent Literature 3 discloses miR-125a-3p, miR-211-3 and the like as biomarkers for ovarian cancers using human sample tissues.
Patent Literature 4 discloses a method for discriminating endometriosis-derived ovarian cancer and serous ovarian cancer from endometriosis using miR-191-5p, miR-652-5p, miR-744-5p, miR-1246 and the like in plasma.
Non-Patent Literature 1 discloses miR-92a, miR-128, miR-191-5p, miR-296-5p, miR-320a, miR-625-3p and the like in blood as circulating extracellular miRNAs involved in ovarian cancer.
Non-Patent Literature 2 discloses that there are differences in expression levels of miR-22, miR-45 and the like as a result of analyzing miRNAs from serum samples of ovarian cancer patients, benign disease patients and healthy subjects, using a next generation sequencer.
Non-Patent Literature 3 discloses that miR-23a/b, miR-92, miR-125a, miR-296-5p, miR-320, miR-422a, miR-663a and the like are present in the body fluid endoplasmic reticulum of ovarian cancer patients.
Non-Patent Literature 4 reports that the CA-125 blood marker had a sensitivity of 77.4% and a specificity of 70.8% for detecting ovarian cancer.

### PRIOR ART LITERATURE

### Patent Literature

Patent Literature 1: Published U.S. Patent Application No. 2016/0312301
Patent Literature 2: JP Patent Publication (Kokai) No. 2010-154843 A (2010)
Patent Literature 3: JP Patent Publication (Kohyo) No. 2010-538610 A (2010)
Patent Literature 4: International Publication No. WO 2013/148151

### Non-Patent Literature

Non-Patent Literature 1: Nakamura K. et al., 2016, Molecular Cancer. Vol. 15, (1):48
Non-Patent Literature 2: Ji T. et al., 2014, Asian Pacific Journal of Cancer Prevention. Vol. 15, (4):1739
Non-Patent Literature 3: Giusti I. et al., 2013, BioMed Research International. Vol. 2013, 703048 Non-Patent Literature 4: Kim Y W. et al., 2012, PLoS One. Vol. 7, (9): e44960

### SUMMARY OF INVENTION

### Problem to be Solved by Invention

An object of the present invention is to find novel tumor markers for ovarian tumor and to provide a method that can effectively detect ovarian tumor using nucleic acids for detecting the markers.

It is difficult to detect ovarian tumor based on subjective symptoms and the screening test method for ovarian tumor is said to be only two, transvaginal ultrasound test and a CA-125 tumor marker test. However, the transvaginal ultrasound test can detect a tumor using ultrasound from within the vagina but the ultrasound has a limited reachable range and thus may fail to detect a tumor present distant from the vagina. Additionally, CA-125 is a protein in blood and is said to increase in ovarian tumor patients but more often increases for different reasons from ovarian tumor, while a CA-125 level does not increase in some ovarian tumor patients thus making the CA-125 test not an effective screening test. Further, as described above, Non-Patent Literature 4 reports that the CA-125 had a sensitivity of 77.4% and a specificity of 70.8% for detecting ovarian cancer.

Additionally, it is known that the transvaginal ultrasound test and CA-125 tumor marker test, even when carried out, do not lead to the reduction in mortality by ovarian cancer.

Further, as described below, there are reports, albeit at a research stage, on the determination of ovarian tumor using the expression levels of miRNAs in biological samples including blood, none of which, however, have yet been brought into practical use.

Patent Literature 1 discloses a method for detecting ovarian cancer or predicting prognosis using miR-191, miR-24, miR-320, miR-328, miR-625-3p, miR-483-5p, miR-92a, miR-1290 and the like in plasma, but these miRNAs are expressed also in control groups and unsuitable for the method for detecting the presence or absence of ovarian cancer. Additionally, the number of samples subjected to the test is as few as several tens of cases and thus the marker has low reliability.

Patent Literature 2 discloses miRNA such as miR-135a-3 and the like in blood as biomarkers for gynecologic cancers, however, it discloses only miRNA expression differences relevant to uterine cancer and does not describe the specific detection performance, such as accuracy, sensitivity, or specificity, for determining ovarian tumor, making these miRNAs poor in industrially practical use.

Patent Literature 3 discloses miRNAs such as miR-125a-3p, miR-211-3 and the like as biomarkers for ovarian cancers using human sample tissues, however, obtaining tissue samples requires tissue resection by surgery, and this step causes a physical burden on a patient, hence not preferable as a test method. Additionally, the literature does not describe the specific detection performance, such as accuracy, sensitivity, or specificity, for determining ovarian cancer, making these miRNAs poor in industrially practical use.

Patent Literature 4 discloses a method for determining, endometriosis-derived ovarian cancer and serous ovarian cancer from endometriosis using miR-191-5p, miR-652-5p, miR-744-5p, miR-1246 and the like in plasma, however, it does not disclose the distinguishment of ovarian cancer from other cancers, and consequently there is a risk for mistakenly detecting other cancers for ovarian cancer. Additionally, the number of samples subjected to the test is as few as several tens of cases and thus the marker has low reliability.

Non-Patent Literature 1 discloses miR-92a, miR-128, miR-191-5p, miR-296-5p, miR-320a, miR-625-3p and the like in blood as circulating extracellular miRNAs involved in ovarian cancer. However, a detection sensitivity for epithelial ovarian cancer by a combination of miR-205 and let-7f is as low as 62.4%, concerning overlooking ovarian cancer. Additionally, the performance of the discriminant between ovarian cancer and healthy subjects in combination with other miRNAs is also partially disclosed but other cancer patients were not included in the research, risking misjudgement of other cancers for ovarian cancer, possibly leading to needless extra examination and a delay in detection of essential cancer, hence not desirable.

Non-Patent Literature 2 discloses that there are differences in expression levels of miRNAs such as miR-22, miR-45 and the like as a result of analyzing miRNAs from serum samples of ovarian cancer patients, benign disease patients and healthy subjects by a next generation sequencer, however, does not describe the specific detection performance, such as accuracy, sensitivity, or specificity, for determining ovarian cancer, making these miRNAs poor in industrially practical use.

Non-Patent Literature 3 discloses that miRNAs such as miR-23a/b, miR-92, miR-125a, miR-296-5p, miR-320, miR-422a, miR-663a and the like are present in the body fluid endoplasmic reticulum of ovarian cancer patients, however, does not describe the specific detection performance, such as accuracy, sensitivity, or specificity, for determining ovarian tumor, making these miRNAs poor in industrially practical use.

As mentioned above, the existing tumor markers exhibit low performance in the detection of ovarian tumor, or neither performance nor detection methods are specifically shown as to the markers at a research stage. Therefore, use of these markers might lead to carry out needless extra examination due to the false detection of healthy subjects as being ovarian tumor patients, or might waste therapeutic opportunity because of overlooking ovarian tumor patients. Furthermore, the collection of ovarian tissues for measuring the tumor markers is highly invasive to patients and is not favorable. Hence, there is a demand for a highly accurate ovarian cancer marker that is detectable from blood, which can be collected in less invasive manner, and is capable of correctly determining an ovarian tumor patient as an ovarian tumor patient and a healthy subject as a healthy subject. Particularly, a highly sensitive ovarian cancer marker capable of detecting ovarian cancer at an early stage of progression is desired because ovarian cancer can dramatically reduce a recurrence risk when detected and treated early, and women can save a chance for future childbirth if the treatment is only partial resection surgery. Furthermore, when a simpler primary screening test for ovarian tumor is provided, a consultation rate of the secondary test is expected to increase.

### Means for Solution of Problem

The present inventors have conducted diligent studies to attain the object and consequently completed the present invention by finding miRNA gene markers usable as markers for detection of ovarian tumor from blood, which can be collected with limited invasiveness, and finding that ovarian tumor can be significantly, preferably specifically, detected by using nucleic acids for detecting such markers, for example, at least one, or at least two, nucleic acids selected from probes capable of specifically binding to any of these markers and primers which amplify these markers.

### SUMMARY OF INVENTION

The present invention has the following features:

(1) A kit for detection of ovarian tumor, comprising a nucleic acid(s) capable of specifically binding to at least one polynucleotide selected from the following ovarian tumor markers: miR-4675, miR-4783-3p, miR-1228-5p, miR-4532, miR-6802-5p, miR-6784-5p, miR-3940-5p, miR-1307-3p, miR-8073, miR-3184-5p, miR-1233-5p, miR-6088, miR-5195-3p, miR-320b, miR-4649-5p, miR-6800-5p, miR-1343-3p, miR-4730, miR-6885-5p, miR-5100, miR-1203, miR-6756-5p, miR-373-5p, miR-1268a, miR-1260b, miR-4258, miR-4697-5p, miR-1469, miR-4515, miR-6861-5p, miR-6821-5p, miR-575, miR-6805-5p, miR-4758-5p, miR-3663-3p, miR-4530, miR-6798-5p, miR-6781-5p, miR-885-3p, miR-1273g-3p, miR-4787-3p, miR-4454, miR-4706, miR-1249-3p, miR-887-3p, miR-6786-5p, miR-1238-5p, miR-6749-5p, miR-6729-5p, miR-6825-5p, miR-663b, miR-6858-5p, miR-4690-5p, miR-6765-5p, miR-4710, miR-6775-5p, miR-371a-5p, miR-6816-5p, miR-296-3p, miR-7977, miR-8069, miR-6515-3p, miR-4687-5p, miR-1343-5p, miR-7110-5p, miR-4525, miR-3158-5p, miR-6787-5p, miR-614, miR-4689, miR-1185-2-3p, miR-1268b, miR-1228-3p, miR-1185-1-3p, miR-940, miR-939-5p, miR-6757-5p, miR-1275, miR-5001-5p, miR-6826-5p, miR-6765-3p, miR-3679-3p, miR-4718, miR-4286, miR-8059, miR-4447, miR-4448, miR-658, miR-6766-3p, miR-197-5p, miR-6887-5p, miR-6742-5p, miR-6729-3p, miR-5090, miR-7975, miR-4505, miR-6889-5p, miR-4708-3p, miR-6131, miR-1225-3p, miR-6132, miR-4734, miR-3194-3p, miR-638, miR-2467-3p, miR-4728-5p, miR-5572, miR-6789-5p, miR-8063, miR-4429, miR-6840-3p, miR-4476, miR-675-5p, miR-711, miR-6875-5p, miR-3160-5p, miR-1908-5p, miR-6726-5p, miR-1913, miR-8071, miR-3648, miR-4732-5p, miR-4787-5p, miR-3917, miR-619-5p, miR-1231, miR-342-5p, miR-4433a-5p, miR-6766-5p, miR-4707-5p, miR-7114-5p, miR-6872-3p, miR-6780b-5p, miR-7845-5p, miR-6798-3p, miR-665, miR-6848-5p, miR-5008-5p, miR-4294, miR-6511a-5p, miR-4435, miR-4747-3p, miR-6880-3p, miR-6869-5p, miR-7150, miR-1260a, miR-6877-5p, miR-6721-5p, miR-4656, miR-1229-5p, miR-4433a-3p, miR-4274, miR-4419b, miR-4674, miR-6893-5p, miR-6763-3p, miR-6762-5p, miR-6738-5p, miR-4513, miR-6746-5p, miR-6880-5p, miR-4736, miR-718, miR-6717-5p, miR-7847-3p, miR-760, miR-1199-5p, miR-6813-5p, miR-6769a-5p, miR-1193, miR-7108-3p, miR-6741-5p, miR-4298, miR-6796-3p, miR-4750-5p, miR-6785-5p, miR-1292-3p, miR-4749-3p, miR-6800-3p, miR-4722-5p, miR-4746-3p, miR-4450, miR-6795-5p, miR-365a-5p, miR-498, miR-6797-5p, miR-1470, miR-6851-5p, miR-1247-3p, miR-5196-5p, miR-208a-5p, miR-6842-5p, miR-150-3p, miR-4534, miR-3135b, miR-3131, miR-4792, miR-6510-5p, miR-504-3p, miR-3619-3p, miR-671-5p, miR-4667-5p, miR-4430, miR-3195, miR-3679-5p, miR-6076, miR-6515-5p, miR-6820-5p, miR-4634, miR-187-5p, miR-6763-5p, miR-1908-3p, miR-1181, miR-6782-5p, miR-5010-5p, miR-6870-5p, miR-6124, miR-1249-5p, miR-6511b-5p, miR-1254, miR-4727-3p, miR-4259, miR-4771, miR-3622a-5p, miR-4480, miR-4740-5p, miR-6777-5p, miR-6794-5p, miR-4687-3p, miR-6743-5p, miR-6771-5p, miR-3141, miR-3162-5p, miR-4271, miR-1227-5p, miR-4257, miR-4270, miR-4516, miR-4651, miR-4725-3p, miR-6125, miR-6732-5p, miR-6791-5p, miR-6819-5p, miR-6891-5p, miR-7108-5p, miR-7109-5p, miR-642b-3p, and miR-642a-3p, or to a complementary strand of the polynucleotide.

(2) The kit according to (1), wherein miR-4675 is hsa-miR-4675, miR-4783-3p is hsa-miR-4783-3p, miR-1228-5p is hsa-miR-1228-5p, miR-4532 is hsa-miR-4532, miR-6802-5p is hsa-miR-6802-5p, miR-6784-5p is hsa-miR-6784-5p, miR-3940-5p is hsa-miR-3940-5p, miR-1307-3p is hsa-miR-1307-3p, miR-8073 is hsa-miR-8073, miR-3184-5p is hsa-miR-3184-5p, miR-1233-5p is hsa-miR-1233-5p, miR-6088 is hsa-miR-6088, miR-5195-3p is hsa-miR-5195-3p, miR-320b is hsa-miR-320b, miR-4649-5p is hsa-miR-4649-5p, miR-6800-5p is hsa-miR-6800-5p, miR-1343-3p is hsa-miR-1343-3p, miR-4730 is hsa-miR-4730, miR-6885-5p is hsa-miR-6885-5p, miR-5100 is hsa-miR-5100, miR-1203 is hsa-miR-1203, miR-6756-5p is hsa-miR-6756-5p, miR-373-5p is hsa-miR-373-5p, miR-1268a is hsa-miR-1268a, miR-1260b is hsa-miR-1260b, miR-4258 is hsa-miR-4258, miR-4697-5p is hsa-miR-4697-5p, miR-1469 is hsa-miR-1469, miR-4515 is hsa-miR-4515, miR-6861-5p is hsa-miR-6861-5p, miR-6821-5p is hsa-miR-6821-5p, miR-575 is hsa-miR-575, miR-6805-5p is hsa-miR-6805-5p, miR-4758-5p is hsa-miR-4758-5p, miR-3663-3p is hsa-miR-3663-3p, miR-4530 is hsa-miR-4530, miR-6798-5p is hsa-miR-6798-5p, miR-6781-5p is hsa-miR-6781-5p, miR-885-3p is hsa-miR-885-3p, miR-1273g-3p is hsa-miR-1273g-3p, miR-4787-3p is hsa-miR-4787-3p, miR-4454 is hsa-miR-4454, miR-4706 is hsa-miR-4706, miR-1249-3p is hsa-miR-1249-3p, miR-887-3p is hsa-miR-887-3p, miR-6786-5p is hsa-miR-6786-5p, miR-1238-5p is hsa-miR-1238-5p, miR-6749-5p is hsa-miR-6749-5p, miR-6729-5p is hsa-miR-6729-5p, miR-6825-5p is hsa-miR-6825-5p, miR-663b is hsa-miR-663b, miR-6858-5p is hsa-miR-6858-5p, miR-4690-5p is hsa-miR-4690-5p, miR-6765-5p is hsa-miR-6765-5p, miR-4710 is hsa-miR-4710, miR-6775-5p is hsa-miR-6775-5p, miR-371a-5p is hsa-miR-371a-5p, miR-6816-5p is hsa-miR-6816-5p, miR-296-3p is hsa-miR-296-3p, miR-7977 is hsa-miR-7977, miR-8069 is hsa-miR-8069, miR-6515-3p is hsa-miR-6515-3p, miR-4687-5p is hsa-miR-4687-5p, miR-1343-5p is hsa-miR-1343-5p, miR-7110-5p is hsa-miR-7110-5p, miR-4525 is hsa-miR-4525, miR-3158-5p is hsa-miR-3158-5p, miR-6787-5p is hsa-miR-6787-5p, miR-614 is hsa-miR-614, miR-4689 is hsa-miR-4689, miR-1185-2-3p is hsa-miR-1185-2-3p, miR-1268b is hsa-miR-1268b, miR-1228-3p is hsa-miR-1228-3p, miR-1185-1-3p is hsa-miR-1185-1-3p, miR-940 is hsa-miR-940, miR-939-5p is hsa-miR-939-5p, miR-6757-5p is hsa-miR-6757-5p, miR-1275 is hsa-miR-1275, miR-5001-5p is hsa-miR-5001-5p, miR-6826-5p is hsa-miR-6826-5p, miR-6765-3p is hsa-miR-6765-3p, miR-3679-3p is hsa-miR-3679-3p, miR-4718 is hsa-miR-4718, miR-4286 is hsa-miR-4286, miR-8059 is hsa-miR-8059, miR-4447 is hsa-miR-4447, miR-4448 is hsa-miR-4448, miR-658 is hsa-miR-658, miR-6766-3p is hsa-miR-6766-3p, miR-197-5p is hsa-miR-197-5p, miR-6887-5p is hsa-miR-6887-5p, miR-6742-5p is hsa-miR-6742-5p, miR-6729-3p is hsa-miR-6729-3p, miR-5090 is hsa-miR-5090, miR-7975 is hsa-miR-7975, miR-4505 is hsa-miR-4505, miR-6889-5p is hsa-miR-6889-5p, miR-4708-3p is hsa-miR-4708-3p, miR-6131 is hsa-miR-6131, miR-1225-3p is hsa-miR-1225-3p, miR-6132 is hsa-miR-6132, miR-4734 is hsa-miR-4734, miR-3194-3p is hsa-miR-3194-3p, miR-638 is hsa-miR-638, miR-2467-3p is hsa-miR-2467-3p, miR-4728-5p is hsa-miR-4728-5p, miR-5572 is hsa-miR-5572, miR-6789-5p is hsa-miR-6789-5p, miR-8063 is hsa-miR-8063, miR-4429 is hsa-miR-4429, miR-6840-3p is hsa-miR-6840-3p, miR-4476 is hsa-miR-4476, miR-675-5p is hsa-miR-675-5p, miR-711 is hsa-miR-711, miR-6875-5p is hsa-miR-6875-5p, miR-3160-5p is hsa-miR-3160-5p, miR-1908-5p is hsa-miR-1908-5p, miR-6726-5p is hsa-miR-6726-5p, miR-1913 is hsa-miR-1913, miR-8071 is hsa-miR-8071, miR-3648 is hsa-miR-3648, miR-4732-5p is hsa-miR-4732-5p, miR-4787-5p is hsa-miR-4787-5p, miR-3917 is hsa-miR-3917, miR-619-5p is hsa-miR-619-5p, miR-1231 is hsa-miR-1231, miR-342-5p is hsa-miR-342-5p, miR-4433a-5p is hsa-miR-4433a-5p, miR-6766-5p is hsa-miR-6766-5p, miR-4707-5p is hsa-miR-4707-5p, miR-7114-5p is hsa-miR-7114-5p, miR-6872-3p is hsa-miR-6872-3p, miR-6780b-5p is hsa-miR-6780b-5p, miR-7845-5p is hsa-miR-7845-5p, miR-6798-3p is hsa-miR-6798-3p, miR-665 is hsa-miR-665, miR-6848-5p is hsa-miR-6848-5p, miR-5008-5p is hsa-miR-5008-5p, miR-4294 is hsa-miR-4294, miR-6511a-5p is hsa-miR-6511a-5p, miR-4435 is hsa-miR-4435, miR-4747-3p is hsa-miR-4747-3p, miR-6880-3p is hsa-miR-6880-3p, miR-6869-5p is hsa-miR-6869-5p, miR-7150 is hsa-miR-7150, miR-1260a is hsa-miR-1260a, miR-6877-5p is hsa-miR-6877-5p, miR-6721-5p is hsa-miR-6721-5p, miR-4656 is hsa-miR-4656, miR-1229-5p is hsa-miR-1229-5p, miR-4433a-3p is hsa-miR-4433a-3p, miR-4274 is hsa-miR-4274, miR-4419b is hsa-miR-4419b, miR-4674 is hsa-miR-4674, miR-6893-5p is hsa-miR-6893-5p, miR-6763-3p is hsa-miR-6763-3p, miR-6762-5p is hsa-miR-6762-5p, miR-6738-5p is hsa-miR-6738-5p, miR-4513 is hsa-miR-4513, miR-6746-5p is hsa-miR-6746-5p, miR-6880-5p is hsa-miR-6880-5p, miR-4736 is hsa-miR-4736, miR-718 is hsa-miR-718, miR-6717-5p is hsa-miR-6717-5p, miR-7847-3p is hsa-miR-7847-3p, miR-760 is hsa-miR-760, miR-1199-5p is hsa-miR-1199-5p, miR-6813-5p is hsa-miR-6813-5p, miR-6769a-5p is hsa-miR-6769a-5p, miR-1193 is hsa-miR-1193, miR-7108-3p is hsa-miR-7108-3p, miR-6741-5p is hsa-miR-6741-5p, miR-4298 is hsa-miR-4298, miR-6796-3p is hsa-miR-6796-3p, miR-4750-5p is hsa-miR-4750-5p, miR-6785-5p is hsa-miR-6785-5p, miR-1292-3p is hsa-miR-1292-3p, miR-4749-3p is hsa-miR-4749-3p, miR-6800-3p is hsa-miR-6800-3p, miR-722-5p is hsa-miR-4722-5p, miR-4746-3p is hsa-miR-4746-3p, miR-4450 is hsa-miR-4450, miR-6795-5p is hsa-miR-6795-5p, miR-365a-5p is hsa-miR-365a-5p, miR-498 is hsa-miR-498, miR-6797-5p is hsa-miR-6797-5p, miR-1470 is hsa-miR-1470, miR-6851-5p is hsa-miR-6851-5p, miR-1247-3p is hsa-miR-1247-3p, miR-5196-5p is hsa-miR-5196-5p, miR-208a-5p is hsa-miR-208a-5p, miR-6842-5p is hsa-miR-6842-5p, miR-150-3p is hsa-miR-150-3p, miR-4534 is hsa-miR-4534, miR-3135b is hsa-miR-3135b, miR-3131 is hsa-miR-3131, miR-4792 is hsa-miR-4792, miR-6510-5p is hsa-miR-6510-5p, miR-504-3p is hsa-miR-504-3p, miR-3619-3p is hsa-miR-3619-3p, miR-671-5p is hsa-miR-671-5p, miR-4667-5p is hsa-miR-4667-5p, miR-4430 is hsa-miR-4430, miR-3195 is hsa-miR-3195, miR-3679-5p is hsa-miR-3679-5p, miR-6076 is hsa-miR-6076, miR-6515-5p is hsa-miR-6515-5p, miR-6820-5p is hsa-miR-6820-5p, miR-4634 is hsa-miR-4634, miR-187-5p is hsa-miR-187-5p, miR-6763-5p is hsa-miR-6763-5p, miR-1908-3p is hsa-miR-1908-3p, miR-1181 is hsa-miR-1181, miR-6782-5p is hsa-miR-6782-5p, miR-5010-5p is hsa-miR-5010-5p, miR-6870-5p is hsa-miR-6870-5p, miR-6124 is hsa-miR-6124, miR-1249-5p is hsa-miR-1249-5p, miR-6511b-5p is hsa-miR-6511b-5p, miR-1254 is hsa-miR-1254, miR-4727-3p is hsa-miR-4727-3p, miR-4259 is hsa-miR-4259, miR-4771 is hsa-miR-4771, miR-3622a-5p is hsa-miR-3622a-5p, miR-4480 is hsa-miR-4480, miR-4740-5p is hsa-miR-4740-5p, miR-6777-5p is hsa-miR-6777-5p, miR-6794-5p is hsa-miR-6794-5p, miR-4687-3p is hsa-miR-4687-3p, miR-6743-5p is hsa-miR-6743-5p, miR-6771-5p is hsa-miR-6771-5p, miR-3141 is hsa-miR-3141, miR-3162-5p is hsa-miR-3162-5p, miR-4271 is hsa-miR-4271, miR-1227-5p is hsa-miR-1227-5p, miR-4257 is hsa-miR-4257, miR-4270 is hsa-miR-4270, miR-4516 is hsa-miR-4516, miR-4651 is hsa-miR-4651, miR-4725-3p is hsa-miR-4725-3p, miR-6125 is hsa-miR-6125, miR-6732-5p is hsa-miR-6732-5p, miR-6791-5p is hsa-miR-6791-5p, miR-6819-5p is hsa-miR-6819-5p, miR-6891-5p is hsa-miR-6891-5p, miR-7108-5p is hsa-miR-7108-5p, miR-7109-5p is hsa-miR-7109-5p, miR-642b-3p is hsa-miR-642b-3p, and miR-642a-3p is hsa-miR-642a-3p.

(3) The kit according to (1) or (2), wherein the nucleic acid(s) is a polynucleotide(s) selected from the group consisting of the following polynucleotides (a) to (e):
(a) a polynucleotide consisting of a nucleotide sequence represented by any of SEQ ID NOs: 1 to 247, 251, and 268 or a nucleotide sequence derived from the nucleotide sequence by the replacement of u with t, a variant thereof, a derivative thereof, or a fragment thereof comprising 15 or more consecutive nucleotides;
(b) a polynucleotide comprising a nucleotide sequence represented by any of SEQ ID NOs: 1 to 247, 251, and 268;
(c) a polynucleotide consisting of a nucleotide sequence complementary to a nucleotide sequence represented by any of SEQ ID NOs: 1 to 247, 251, and 268 or a nucleotide sequence derived from the nucleotide sequence by the replacement of u with t, a variant thereof, a derivative thereof, or a fragment thereof comprising 15 or more consecutive nucleotides;
(d) a polynucleotide comprising a nucleotide sequence complementary to a nucleotide sequence represented by any of SEQ ID NOs: 1 to 247, 251, and 268 or a nucleotide sequence derived from the nucleotide sequence by the replacement of u with t; and (e) a polynucleotide hybridizing under stringent conditions to any of the polynucleotides (a) to (d).

(4) The kit according to any of (1) to (3), wherein the kit further comprises a nucleic acid(s) capable of specifically binding to at least one polynucleotide selected from the group consisting of other ovarian tumor markers: miR-320a, miR-663a, miR-328-5p, miR-128-2-5p, miR-125a-3p, miR-191-5p, miR-92b-5p, miR-296-5p, miR-1246, miR-92a-2-5p, miR-128-1-5p, miR-1290, miR-211-3p, miR-744-5p, miR-135a-3p, miR-451a, miR-625-3p, miR-92a-3p, miR-422a, miR-483-5p, miR-652-5p, miR-24-3p, miR-23b-3p, miR-23a-3p, miR-92b-3p, and miR-22-3p, or to a complementary strand of the polynucleotide.

(5) The kit according to (4), wherein miR-320a is hsa-miR-320a, miR-663a is hsa-miR-663a, miR-328-5p is hsa-miR-328-5p, miR-128-2-5p is hsa-miR-128-2-5p, miR-125a-3p is hsa-miR-125a-3p, miR-191-5p is hsa-miR-191-5p, miR-92b-5p is hsa-miR-92b-5p, miR-296-5p is hsa-miR-296-5p, miR-1246 is hsa-miR-1246, miR-92a-2-5p is hsa-miR-92a-2-5p, miR-128-1-5p is hsa-miR-128-1-5p, miR-1290 is hsa-miR-1290, miR-211-3p is hsa-miR-211-3p, miR-744-5p is hsa-miR-744-5p, miR-135a-3p is hsa-miR-135a-3p, miR-451a is hsa-miR-451a, miR-625-3p is hsa-miR-625-3p, miR-92a-3p is hsa-miR-92a-3p, miR-422a is hsa-miR-422a, miR-483-5p is hsa-miR-483-5p, miR-652-5p is hsa-miR-652-5p, miR-24-3p is hsa-miR-24-3p, miR-23b-3p is hsa-miR-23b-3p, miR-23a-3p is hsa-miR-23a-3p, miR-92b-3p is hsa-miR-92b-3p, and miR-22-3p is hsa-miR-22-3p.

(6) The kit according to (4) or (5), wherein the nucleic acid(s) is a polynucleotide(s) selected from the group consisting of the following polynucleotides (f) to (j):
(f) a polynucleotide consisting of a nucleotide sequence represented by any of SEQ ID NOs: 248 to 250, 252 to 267, and 269 to 275 or a nucleotide sequence derived from the nucleotide sequence by the replacement of u with t, a variant thereof, a derivative thereof, or a fragment thereof comprising 15 or more consecutive nucleotides;
(g) a polynucleotide comprising a nucleotide sequence represented by any of SEQ ID NOs: 248 to 250, 252 to 267, and 269 to 275;
(h) a polynucleotide consisting of a nucleotide sequence complementary to a nucleotide sequence represented by any of SEQ ID NOs: 248 to 250, 252 to 267, and 269 to 275 or a nucleotide sequence derived from the nucleotide sequence by the replacement of u with t, a variant thereof, a derivative thereof, or a fragment thereof comprising 15 or more consecutive nucleotides;
(i) a polynucleotide comprising a nucleotide sequence complementary to a nucleotide sequence represented by any of SEQ ID NOs: 248 to 250, 252 to 267, and 269 to 275 or a nucleotide sequence derived from the nucleotide sequence by the replacement of u with t; and
j) a polynucleotide hybridizing under stringent conditions to any of the polynucleotides (f) to (i).

(7) The kit according to any of (1) to (6), wherein the kit comprises at least two nucleic acids capable of specifically binding to at least two polynucleotides selected from all of the ovarian tumor markers according to (1) or (2), or complementary strands of the polynucleotides, respectively.

(8) A device for detection of ovarian tumor, comprising a nucleic acid(s) capable of specifically binding to at least one polynucleotide selected from the following ovarian tumor markers: miR-4675, miR-4783-3p, miR-1228-5p, miR-4532, miR-6802-5p, miR-6784-5p, miR-3940-5p, miR-1307-3p, miR-8073, miR-3184-5p, miR-1233-5p, miR-6088, miR-5195-3p, miR-320b, miR-4649-5p, miR-6800-5p, miR-1343-3p, miR-4730, miR-6885-5p, miR-5100, miR-1203, miR-6756-5p, miR-373-5p, miR-1268a, miR-1260b, miR-4258, miR-4697-5p, miR-1469, miR-4515, miR-6861-5p, miR-6821-5p, miR-575, miR-6805-5p, miR-4758-5p, miR-3663-3p, miR-4530, miR-6798-5p, miR-6781-5p, miR-885-3p, miR-1273g-3p, miR-4787-3p, miR-4454, miR-4706, miR-1249-3p, miR-887-3p, miR-6786-5p, miR-1238-5p, miR-6749-5p, miR-6729-5p, miR-6825-5p, miR-663b, miR-6858-5p, miR-4690-5p, miR-6765-5p, miR-4710, miR-6775-5p, miR-371a-5p, miR-6816-5p, miR-296-3p, miR-7977, miR-8069, miR-6515-3p, miR-4687-5p, miR-1343-5p, miR-7110-5p, miR-4525, miR-3158-5p, miR-6787-5p, miR-614, miR-4689, miR-1185-2-3p, miR-1268b, miR-1228-3p, miR-1185-1-3p, miR-940, miR-939-5p, miR-6757-5p, miR-1275, miR-5001-5p, miR-6826-5p, miR-6765-3p, miR-3679-3p, miR-4718, miR-4286, miR-8059, miR-4447, miR-4448, miR-658, miR-6766-3p, miR-197-5p, miR-6887-5p, miR-6742-5p, miR-6729-3p, miR-5090, miR-7975, miR-4505, miR-6889-5p, miR-4708-3p, miR-6131, miR-1225-3p, miR-6132, miR-4734, miR-3194-3p, miR-638, miR-2467-3p, miR-4728-5p, miR-5572, miR-6789-5p, miR-8063, miR-4429, miR-6840-3p, miR-4476, miR-675-5p, miR-711, miR-6875-5p, miR-3160-5p, miR-1908-5p, miR-6726-5p, miR-1913, miR-8071, miR-3648, miR-4732-5p, miR-4787-5p, miR-3917, miR-619-5p, miR-1231, miR-342-5p, miR-4433a-5p, miR-6766-5p, miR-4707-5p, miR-7114-5p, miR-6872-3p, miR-6780b-5p, miR-7845-5p, miR-6798-3p, miR-665, miR-6848-5p, miR-5008-5p, miR-4294, miR-6511a-5p, miR-4435, miR-4747-3p, miR-6880-3p, miR-6869-5p, miR-7150, miR-1260a, miR-6877-5p, miR-6721-5p, miR-4656, miR-1229-5p, miR-4433a-3p, miR-4274, miR-4419b, miR-4674, miR-6893-5p, miR-6763-3p, miR-6762-5p, miR-6738-5p, miR-4513, miR-6746-5p, miR-6880-5p, miR-4736, miR-718, miR-6717-5p, miR-7847-3p, miR-760, miR-1199-5p, miR-6813-5p, miR-6769a-5p, miR-1193, miR-7108-3p, miR-6741-5p, miR-4298, miR-6796-3p, miR-4750-5p, miR-6785-5p, miR-1292-3p, miR-4749-3p, miR-6800-3p, miR-4722-5p, miR-4746-3p, miR-4450, miR-6795-5p, miR-365a-5p, miR-498, miR-6797-5p, miR-1470, miR-6851-5p, miR-1247-3p, miR-5196-5p, miR-208a-5p, miR-6842-5p, miR-150-3p, miR-4534, miR-3135b, miR-3131, miR-4792, miR-6510-5p, miR-504-3p, miR-3619-3p, miR-671-5p, miR-4667-5p, miR-4430, miR-3195, miR-3679-5p, miR-6076, miR-6515-5p, miR-6820-5p, miR-4634, miR-187-5p, miR-6763-5p, miR-1908-3p, miR-1181, miR-6782-5p, miR-5010-5p, miR-6870-5p, miR-6124, miR-1249-5p, miR-6511b-5p, miR-1254, miR-4727-3p, miR-4259, miR-4771, miR-3622a-5p, miR-4480, miR-4740-5p, miR-6777-5p, miR-6794-5p, miR-4687-3p, miR-6743-5p, miR-6771-5p, miR-3141, miR-3162-5p, miR-4271, miR-1227-5p, miR-4257, miR-4270, miR-4516, miR-4651, miR-4725-3p, miR-6125, miR-6732-5p, miR-6791-5p, miR-6819-5p, miR-6891-5p, miR-7108-5p, miR-7109-5p, miR-642b-3p, and miR-642a-3p, or to a complementary strand of the polynucleotide.

(9) The device according to (8), wherein miR-4675 is hsa-miR-4675, miR-4783-3p is hsa-miR-4783-3p, miR-1228-5p is hsa-miR-1228-5p, miR-4532 is hsa-miR-4532, miR-6802-5p is hsa-miR-6802-5p, miR-6784-5p is hsa-miR-6784-5p, miR-3940-5p is hsa-miR-3940-5p, miR-1307-3p is hsa-miR-1307-3p, miR-8073 is hsa-miR-8073, miR-3184-5p is hsa-miR-3184-5p, miR-1233-5p is hsa-miR-1233-5p, miR-6088 is hsa-miR-6088, miR-5195-3p is hsa-miR-5195-3p, miR-320b is hsa-miR-320b, miR-4649-5p is hsa-miR-4649-5p, miR-6800-5p is hsa-miR-6800-5p, miR-1343-3p is hsa-miR-1343-3p, miR-4730 is hsa-miR-4730, miR-6885-5p is hsa-miR-6885-5p, miR-5100 is hsa-miR-5100, miR-1203 is hsa-miR-1203, miR-6756-5p is hsa-miR-6756-5p, miR-373-5p is hsa-miR-373-5p, miR-1268a is hsa-miR-1268a, miR-1260b is hsa-miR-1260b, miR-4258 is hsa-miR-4258, miR-4697-5p is hsa-miR-4697-5p, miR-1469 is hsa-miR-1469, miR-4515 is hsa-miR-4515, miR-6861-5p is hsa-miR-6861-5p, miR-6821-5p is hsa-miR-6821-5p, miR-575 is hsa-miR-575, miR-6805-5p is hsa-miR-6805-5p, miR-4758-5p is hsa-miR-4758-5p, miR-3663-3p is hsa-miR-3663-3p, miR-4530 is hsa-miR-4530, miR-6798-5p is hsa-miR-6798-5p, miR-6781-5p is hsa-miR-6781-5p, miR-885-3p is hsa-miR-885-3p, miR-1273g-3p is hsa-miR-1273g-3p, miR-4787-3p is hsa-miR-4787-3p, miR-4454 is hsa-miR-4454, miR-4706 is hsa-miR-4706, miR-1249-3p is hsa-miR-1249-3p, miR-887-3p is hsa-miR-887-3p, miR-6786-5p is hsa-miR-6786-5p, miR-1238-5p is hsa-miR-1238-5p, miR-6749-5p is hsa-miR-6749-5p, miR-6729-5p is hsa-miR-6729-5p, miR-6825-5p is hsa-miR-6825-5p, miR-663b is hsa-miR-663b, miR-6858-5p is hsa-miR-6858-5p, miR-4690-5p is hsa-miR-4690-5p, miR-6765-5p is hsa-miR-6765-5p, miR-4710 is hsa-miR-4710, miR-6775-5p is hsa-miR-6775-5p, miR-371a-5p is hsa-miR-371a-5p, miR-6816-5p is hsa-miR-6816-5p, miR-296-3p is hsa-miR-296-3p, miR-7977 is hsa-miR-7977, miR-8069 is hsa-miR-8069, miR-6515-3p is hsa-miR-6515-3p, miR-4687-5p is hsa-miR-4687-5p, miR-1343-5p is hsa-miR-1343-5p, miR-7110-5p is hsa-miR-7110-5p, miR-4525 is hsa-miR-4525, miR-3158-5p is hsa-miR-3158-5p, miR-6787-5p is hsa-miR-6787-5p, miR-614 is hsa-miR-614, miR-4689 is hsa-miR-4689, miR-1185-2-3p is hsa-miR-1185-2-3p, miR-1268b is hsa-miR-1268b, miR-1228-3p is hsa-miR-1228-3p, miR-1185-1-3p is hsa-miR-1185-1-3p, miR-940 is hsa-miR-940, miR-939-5p is hsa-miR-939-5p, miR-6757-5p is hsa-miR-6757-5p, miR-1275 is hsa-miR-1275, miR-5001-5p is hsa-miR-5001-5p, miR-6826-5p is hsa-miR-6826-5p, miR-6765-3p is hsa-miR-6765-3p, miR-3679-3p is hsa-miR-3679-3p, miR-4718 is hsa-miR-4718, miR-4286 is hsa-miR-4286, miR-8059 is hsa-miR-8059, miR-4447 is hsa-miR-4447, miR-4448 is hsa-miR-4448, miR-658 is hsa-miR-658, miR-6766-3p is hsa-miR-6766-3p, miR-197-5p is hsa-miR-197-5p, miR-6887-5p is hsa-miR-6887-5p, miR-6742-5p is hsa-miR-6742-5p, miR-6729-3p is hsa-miR-6729-3p, miR-5090 is hsa-miR-5090, miR-7975 is hsa-miR-7975, miR-4505 is hsa-miR-4505, miR-6889-5p is hsa-miR-6889-5p, miR-4708-3p is hsa-miR-4708-3p, miR-6131 is hsa-miR-6131, miR-1225-3p is hsa-miR-1225-3p, miR-6132 is hsa-miR-6132, miR-4734 is hsa-miR-4734, miR-3194-3p is hsa-miR-3194-3p, miR-638 is hsa-miR-638, miR-2467-3p is hsa-miR-2467-3p, miR-4728-5p is hsa-miR-4728-5p, miR-5572 is hsa-miR-5572, miR-6789-5p is hsa-miR-6789-5p, miR-8063 is hsa-miR-8063, miR-4429 is hsa-miR-4429, miR-6840-3p is hsa-miR-6840-3p, miR-4476 is hsa-miR-4476, miR-675-5p is hsa-miR-675-5p, miR-711 is hsa-miR-711, miR-6875-5p is hsa-miR-6875-5p, miR-3160-5p is hsa-miR-3160-5p, miR-1908-5p is hsa-miR-1908-5p, miR-6726-5p is hsa-miR-6726-5p, miR-1913 is hsa-miR-1913, miR-8071 is hsa-miR-8071, miR-3648 is hsa-miR-3648, miR-4732-5p is hsa-miR-4732-5p, miR-4787-5p is hsa-miR-4787-5p, miR-3917 is hsa-miR-3917, miR-619-5p is hsa-miR-619-5p, miR-1231 is hsa-miR-1231, miR-342-5p is hsa-miR-342-5p, miR-4433a-5p is hsa-miR-4433a-5p, miR-6766-5p is hsa-miR-6766-5p, miR-4707-5p is hsa-miR-4707-5p, miR-7114-5p is hsa-miR-7114-5p, miR-6872-3p is hsa-miR-6872-3p, miR-6780b-5p is hsa-miR-6780b-5p, miR-7845-5p is hsa-miR-7845-5p, miR-6798-3p is hsa-miR-6798-3p, miR-665 is hsa-miR-665, miR-6848-5p is hsa-miR-6848-5p, miR-5008-5p is hsa-miR-5008-5p, miR-4294 is hsa-miR-4294, miR-6511a-5p is hsa-miR-6511a-5p, miR-4435 is hsa-miR-4435, miR-4747-3p is hsa-miR-4747-3p, miR-6880-3p is hsa-miR-6880-3p, miR-6869-5p is hsa-miR-6869-5p, miR-7150 is hsa-miR-7150, miR-1260a is hsa-miR-1260a, miR-6877-5p is hsa-miR-6877-5p, miR-6721-5p is hsa-miR-6721-5p, miR-4656 is hsa-miR-4656, miR-1229-5p is hsa-miR-1229-5p, miR-4433a-3p is hsa-miR-4433a-3p, miR-4274 is hsa-miR-4274, miR-4419b is hsa-miR-4419b, miR-4674 is hsa-miR-4674, miR-6893-5p is hsa-miR-6893-5p, miR-6763-3p is hsa-miR-6763-3p, miR-6762-5p is hsa-miR-6762-5p, miR-6738-5p is hsa-miR-6738-5p, miR-4513 is hsa-miR-4513, miR-6746-5p is hsa-miR-6746-5p, miR-6880-5p is hsa-miR-6880-5p, miR-4736 is hsa-miR-4736, miR-718 is hsa-miR-718, miR-6717-5p is hsa-miR-6717-5p, miR-7847-3p is hsa-miR-7847-3p, miR-760 is hsa-miR-760, miR-1199-5p is hsa-miR-1199-5p, miR-6813-5p is hsa-miR-6813-5p, miR-6769a-5p is hsa-miR-6769a-5p, miR-1193 is hsa-miR-1193, miR-7108-3p is hsa-miR-7108-3p, miR-6741-5p is hsa-miR-6741-5p, miR-4298 is hsa-miR-4298, miR-6796-3p is hsa-miR-6796-3p, miR-4750-5p is hsa-miR-4750-5p, miR-6785-5p is hsa-miR-6785-5p, miR-1292-3p is hsa-miR-1292-3p, miR-4749-3p is hsa-miR-4749-3p, miR-6800-3p is hsa-miR-6800-3p, miR-722-5p is hsa-miR-4722-5p, miR-4746-3p is hsa-miR-4746-3p, miR-4450 is hsa-miR-4450, miR-6795-5p is hsa-miR-6795-5p, miR-365a-5p is hsa-miR-365a-5p, miR-498 is hsa-miR-498, miR-6797-5p is hsa-miR-6797-5p, miR-1470 is hsa-miR-1470, miR-6851-5p is hsa-miR-6851-5p, miR-1247-3p is hsa-miR-1247-3p, miR-5196-5p is hsa-miR-5196-5p, miR-208a-5p is hsa-miR-208a-5p, miR-6842-5p is hsa-miR-6842-5p, miR-150-3p is hsa-miR-150-3p, miR-4534 is hsa-miR-4534, miR-3135b is hsa-miR-3135b, miR-3131 is hsa-miR-3131, miR-4792 is hsa-miR-4792, miR-6510-5p is hsa-miR-6510-5p, miR-504-3p is hsa-miR-504-3p, miR-3619-3p is hsa-miR-3619-3p, miR-671-5p is hsa-miR-671-5p, miR-4667-5p is hsa-miR-4667-5p, miR-4430 is hsa-miR-4430, miR-3195 is hsa-miR-3195, miR-3679-5p is hsa-miR-3679-5p, miR-6076 is hsa-miR-6076, miR-6515-5p is hsa-miR-6515-5p, miR-6820-5p is hsa-miR-6820-5p, miR-4634 is hsa-miR-4634, miR-187-5p is hsa-miR-187-5p, miR-6763-5p is hsa-miR-6763-5p, miR-1908-3p is hsa-miR-1908-3p, miR-1181 is hsa-miR-1181, miR-6782-5p is hsa-miR-6782-5p, miR-5010-5p is hsa-miR-5010-5p, miR-6870-5p is hsa-miR-6870-5p, miR-6124 is hsa-miR-6124, miR-1249-5p is hsa-miR-1249-5p, miR-6511b-5p is hsa-miR-6511b-5p, miR-1254 is hsa-miR-1254, miR-4727-3p is hsa-miR-4727-3p, miR-4259 is hsa-miR-4259, miR-4771 is hsa-miR-4771, miR-3622a-5p is hsa-miR-3622a-5p, miR-4480 is hsa-miR-4480, miR-4740-5p is hsa-miR-4740-5p, miR-6777-5p is hsa-miR-6777-5p, miR-6794-5p is hsa-miR-6794-5p, miR-4687-3p is hsa-miR-4687-3p, miR-6743-5p is hsa-miR-6743-5p, miR-6771-5p is hsa-miR-6771-5p, miR-3141 is hsa-miR-3141, miR-3162-5p is hsa-miR-3162-5p, miR-4271 is hsa-miR-4271, miR-1227-5p is hsa-miR-1227-5p, miR-4257 is hsa-miR-4257, miR-4270 is hsa-miR-4270, miR-4516 is hsa-miR-4516, miR-4651 is hsa-miR-4651, miR-4725-3p is hsa-miR-4725-3p, miR-6125 is hsa-miR-6125, miR-6732-5p is hsa-miR-6732-5p, miR-6791-5p is hsa-miR-6791-5p, miR-6819-5p is hsa-miR-6819-5p, miR-6891-5p is hsa-miR-6891-5p, miR-7108-5p is hsa-miR-7108-5p, miR-7109-5p is hsa-miR-7109-5p, miR-642b-3p is hsa-miR-642b-3p, and miR-642a-3p is hsa-miR-642a-3p.

(10) The device according to (8) or (9), wherein the nucleic acid(s) is a polynucleotide(s) selected from the group consisting of the following polynucleotides (a) to (e):
(a) a polynucleotide consisting of a nucleotide sequence represented by any of SEQ ID NOs: 1 to 247, 251, and 268 or a nucleotide sequence derived from the nucleotide sequence by the replacement of u with t, a variant thereof, a derivative thereof, or a fragment thereof comprising 15 or more consecutive nucleotides;
(b) a polynucleotide comprising a nucleotide sequence represented by any of SEQ ID NOs: 1 to 247, 251, and 268;
(c) a polynucleotide consisting of a nucleotide sequence complementary to a nucleotide sequence represented by any of SEQ ID NOs: 1 to 247, 251, and 268 or a nucleotide sequence derived from the nucleotide sequence by the replacement of u with t, a variant thereof, a derivative thereof, or a fragment thereof comprising 15 or more consecutive nucleotides;
(d) a polynucleotide comprising a nucleotide sequence complementary to a nucleotide sequence represented by any of SEQ ID NOs: 1 to 247, 251, and 268 or a nucleotide sequence derived from the nucleotide sequence by the replacement of u with t; and (e) a polynucleotide hybridizing under stringent conditions to any of the polynucleotides (a) to (d).

(11) The device according to any of (8) to (10), wherein the device further comprises a nucleic acid(s) capable of specifically binding to at least one polynucleotide selected from the group consisting of other ovarian tumor markers: miR-320a, miR-663a, miR-328-5p, miR-128-2-5p, miR-125a-3p, miR-191-5p, miR-92b-5p, miR-296-5p, miR-1246, miR-92a-2-5p, miR-128-1-5p, miR-1290, miR-211-3p, miR-744-5p, miR-135a-3p, miR-451a, miR-625-3p, miR-92a-3p, miR-422a, miR-483-5p, miR-652-5p, miR-24-3p, miR-23b-3p, miR-23a-3p, miR-92b-3p, and miR-22-3p, or to a complementary strand of the polynucleotide.

(12) The device according to (11), wherein miR-320a is hsa-miR-320a, miR-663a is hsa-miR-663a, miR-328-5p is hsa-miR-328-5p, miR-128-2-5p is hsa-miR-128-2-5p, miR-125a-3p is hsa-miR-125a-3p, miR-191-5p is hsa-miR-191-5p, miR-92b-5p is hsa-miR-92b-5p, miR-296-5p is hsa-miR-296-5p, miR-1246 is hsa-miR-1246, miR-92a-2-5p is hsa-miR-92a-2-5p, miR-128-1-5p is hsa-miR-128-1-5p, miR-1290 is hsa-miR-1290, miR-211-3p is hsa-miR-211-3p, miR-744-5p is hsa-miR-744-5p, miR-135a-3p is hsa-miR-135a-3p, miR-451a is hsa-miR-451a, miR-625-3p is hsa-miR-625-3p, miR-92a-3p is hsa-miR-92a-3p, miR-422a is hsa-miR-422a, miR-483-5p is hsa-miR-483-5p, miR-652-5p is hsa-miR-652-5p, miR-24-3p is hsa-miR-24-3p, miR-23b-3p is hsa-miR-23b-3p, miR-23a-3p is hsa-miR-23a-3p, miR-92b-3p is hsa-miR-92b-3p, and miR-22-3p is hsa-miR-22-3p.

(13) The device according to (11) or (12), wherein the nucleic acid(s) is a polynucleotide(s) selected from the group consisting of the following polynucleotides (f) to j):
(f) a polynucleotide consisting of a nucleotide sequence represented by any of SEQ ID NOs: 248 to 250, 252 to 267, and 269 to 275 or a nucleotide sequence derived from the nucleotide sequence by the replacement of u with t, a variant thereof, a derivative thereof, or a fragment thereof comprising 15 or more consecutive nucleotides;
(g) a polynucleotide comprising a nucleotide sequence represented by any of SEQ ID NOs: 248 to 250, 252 to 267, and 269 to 275;
(h) a polynucleotide consisting of a nucleotide sequence complementary to a nucleotide sequence represented by any of SEQ ID NOs: 248 to 250, 252 to 267, and 269 to 275 or a nucleotide sequence derived from the nucleotide sequence by the replacement of u with t, a variant thereof, a derivative thereof, or a fragment thereof comprising 15 or more consecutive nucleotides;
(i) a polynucleotide comprising a nucleotide sequence complementary to a nucleotide sequence represented by any of SEQ ID NOs: 248 to 250, 252 to 267, and 269 to 275 or a nucleotide sequence derived from the nucleotide sequence by the replacement of u with t; and
j) a polynucleotide hybridizing under stringent conditions to any of the polynucleotides (f) to (i).

(14) The device according to any of (8) to (13), wherein the device is for measurement based on a hybridization technique.

(15) The device according to (14), wherein the hybridization technique is a nucleic acid array technique.

(16) The device according to any of (8) to (15), wherein the device comprises at least two polynucleotides selected from all of the ovarian tumor markers according to (8) or (9), or at least two nucleic acids capable of specifically binding to a complementary strand of the polynucleotide, respectively.

(17) A method for detecting ovarian tumor, comprising: measuring an expression level(s) of at least one polynucleotide selected from the following ovarian tumor markers: miR-4675, miR-4783-3p, miR-1228-5p, miR-4532, miR-6802-5p, miR-6784-5p, miR-3940-5p, miR-1307-3p, miR-8073, miR-3184-5p, miR-1233-5p, miR-6088, miR-5195-3p, miR-320b, miR-4649-5p, miR-6800-5p, miR-1343-3p, miR-4730, miR-6885-5p, miR-5100, miR-1203, miR-6756-5p, miR-373-5p, miR-1268a, miR-1260b, miR-4258, miR-4697-5p, miR-1469, miR-4515, miR-6861-5p, miR-6821-5p, miR-575, miR-6805-5p, miR-4758-5p, miR-3663-3p, miR-4530, miR-6798-5p, miR-6781-5p, miR-885-3p, miR-1273g-3p, miR-4787-3p, miR-4454, miR-4706, miR-1249-3p, miR-887-3p, miR-6786-5p, miR-1238-5p, miR-6749-5p, miR-6729-5p, miR-6825-5p, miR-663b, miR-6858-5p, miR-4690-5p, miR-6765-5p, miR-4710, miR-6775-5p, miR-371a-5p, miR-6816-5p, miR-296-3p, miR-7977, miR-8069, miR-6515-3p, miR-4687-5p, miR-1343-5p, miR-7110-5p, miR-4525, miR-3158-5p, miR-6787-5p, miR-614, miR-4689, miR-1185-2-3p, miR-1268b, miR-1228-3p, miR-1185-1-3p, miR-940, miR-939-5p, miR-6757-5p, miR-1275, miR-5001-5p, miR-6826-5p, miR-6765-3p, miR-3679-3p, miR-4718, miR-4286, miR-8059, miR-4447, miR-4448, miR-658, miR-6766-3p, miR-197-5p, miR-6887-5p, miR-6742-5p, miR-6729-3p, miR-5090, miR-7975, miR-4505, miR-6889-5p, miR-4708-3p, miR-6131, miR-1225-3p, miR-6132, miR-4734, miR-3194-3p, miR-638, miR-2467-3p, miR-4728-5p, miR-5572, miR-6789-5p, miR-8063, miR-4429, miR-6840-3p, miR-4476, miR-675-5p, miR-711, miR-6875-5p, miR-3160-5p, miR-1908-5p, miR-6726-5p, miR-1913, miR-8071, miR-3648, miR-4732-5p, miR-4787-5p, miR-3917, miR-619-5p, miR-1231, miR-342-5p, miR-4433a-5p, miR-6766-5p, miR-4707-5p, miR-7114-5p, miR-6872-3p, miR-6780b-5p, miR-7845-5p, miR-6798-3p, miR-665, miR-6848-5p, miR-5008-5p, miR-4294, miR-6511a-5p, miR-4435, miR-4747-3p, miR-6880-3p, miR-6869-5p, miR-7150, miR-1260a, miR-6877-5p, miR-6721-5p, miR-4656, miR-1229-5p, miR-4433a-3p, miR-4274, miR-4419b, miR-4674, miR-6893-5p, miR-6763-3p, miR-6762-5p, miR-6738-5p, miR-4513, miR-6746-5p, miR-6880-5p, miR-4736, miR-718, miR-6717-5p, miR-7847-3p, miR-760, miR-1199-5p, miR-6813-5p, miR-6769a-5p, miR-1193, miR-7108-3p, miR-6741-5p, miR-4298, miR-6796-3p, miR-4750-5p, miR-6785-5p, miR-1292-3p, miR-4749-3p, miR-6800-3p, miR-4722-5p, miR-4746-3p, miR-4450, miR-6795-5p, miR-365a-5p, miR-498, miR-6797-5p, miR-1470, miR-6851-5p, miR-1247-3p, miR-5196-5p, miR-208a-5p, miR-6842-5p, miR-150-3p, miR-4534, miR-3135b, miR-3131, miR-4792, miR-6510-5p, miR-504-3p, miR-3619-3p, miR-671-5p, miR-4667-5p, miR-4430, miR-3195, miR-3679-5p, miR-6076, miR-6515-5p, miR-6820-5p, miR-4634, miR-187-5p, miR-6763-5p, miR-1908-3p, miR-1181, miR-6782-5p, miR-5010-5p, miR-6870-5p, miR-6124, miR-1249-5p, miR-6511b-5p, miR-1254, miR-4727-3p, miR-4259, miR-4771, miR-3622a-5p, miR-4480, miR-4740-5p, miR-6777-5p, miR-6794-5p, miR-4687-3p, miR-6743-5p, miR-6771-5p, miR-3141, miR-3162-5p, miR-4271, miR-1227-5p, miR-4257, miR-4270, miR-4516, miR-4651, miR-4725-3p, miR-6125, miR-6732-5p, miR-6791-5p, miR-6819-5p, miR-6891-5p, miR-7108-5p, miR-7109-5p, miR-642b-3p, and miR-642a-3p in a sample from a subject; and evaluating in vitro whether or not the subject has ovarian tumor using the measured expression level(s).

(18) A method for detecting ovarian tumor, comprising: measuring an expression level(s) of a target nucleic acid(s) in a sample from a subject using a kit according to any of (1) to (7) or a device according to any of (8) to (16), comprising a nucleic acid(s) capable of specifically binding to at least one or at least two polynucleotides selected from the following ovarian tumor markers: miR-4675, miR-4783-3p, miR-1228-5p, miR-4532, miR-6802-5p, miR-6784-5p, miR-3940-5p, miR-1307-3p, miR-8073, miR-3184-5p, miR-1233-5p, miR-6088, miR-5195-3p, miR-320b, miR-4649-5p, miR-6800-5p, miR-1343-3p, miR-4730, miR-6885-5p, miR-5100, miR-1203, miR-6756-5p, miR-373-5p, miR-1268a, miR-1260b, miR-4258, miR-4697-5p, miR-1469, miR-4515, miR-6861-5p, miR-6821-5p, miR-575, miR-6805-5p, miR-4758-5p, miR-3663-3p, miR-4530, miR-6798-5p, miR-6781-5p, miR-885-3p, miR-1273g-3p, miR-4787-3p, miR-4454, miR-4706, miR-1249-3p, miR-887-3p, miR-6786-5p, miR-1238-5p, miR-6749-5p, miR-6729-5p, miR-6825-5p, miR-663b, miR-6858-5p, miR-4690-5p, miR-6765-5p, miR-4710, miR-6775-5p, miR-371a-5p, miR-6816-5p, miR-296-3p, miR-7977, miR-8069, miR-6515-3p, miR-4687-5p, miR-1343-5p, miR-7110-5p, miR-4525, miR-3158-5p, miR-6787-5p, miR-614, miR-4689, miR-1185-2-3p, miR-1268b, miR-1228-3p, miR-1185-1-3p, miR-940, miR-939-5p, miR-6757-5p, miR-1275, miR-5001-5p, miR-6826-5p, miR-6765-3p, miR-3679-3p, miR-4718, miR-4286, miR-8059, miR-4447, miR-4448, miR-658, miR-6766-3p, miR-197-5p, miR-6887-5p, miR-6742-5p, miR-6729-3p, miR-5090, miR-7975, miR-4505, miR-6889-5p, miR-4708-3p, miR-6131, miR-1225-3p, miR-6132, miR-4734, miR-3194-3p, miR-638, miR-2467-3p, miR-4728-5p, miR-5572, miR-6789-5p, miR-8063, miR-4429, miR-6840-3p, miR-4476, miR-675-5p, miR-711, miR-6875-5p, miR-3160-5p, miR-1908-5p, miR-6726-5p, miR-1913, miR-8071, miR-3648, miR-4732-5p, miR-4787-5p, miR-3917, miR-619-5p, miR-1231, miR-342-5p, miR-4433a-5p, miR-6766-5p, miR-4707-5p, miR-7114-5p, miR-6872-3p, miR-6780b-5p, miR-7845-5p, miR-6798-3p, miR-665, miR-6848-5p, miR-5008-5p, miR-4294, miR-6511a-5p, miR-4435, miR-4747-3p, miR-6880-3p, miR-6869-5p, miR-7150, miR-1260a, miR-6877-5p, miR-6721-5p, miR-4656, miR-1229-5p, miR-4433a-3p, miR-4274, miR-4419b, miR-4674, miR-6893-5p, miR-6763-3p, miR-6762-5p, miR-6738-5p, miR-4513, miR-6746-5p, miR-6880-5p, miR-4736, miR-718, miR-6717-5p, miR-7847-3p, miR-760, miR-1199-5p, miR-6813-5p, miR-6769a-5p, miR-1193, miR-7108-3p, miR-6741-5p, miR-4298, miR-6796-3p, miR-4750-5p, miR-6785-5p, miR-1292-3p, miR-4749-3p, miR-6800-3p, miR-4722-5p, miR-4746-3p, miR-4450, miR-6795-5p, miR-365a-5p, miR-498, miR-6797-5p, miR-1470, miR-6851-5p, miR-1247-3p, miR-5196-5p, miR-208a-5p, miR-6842-5p, miR-150-3p, miR-4534, miR-3135b, miR-3131, miR-4792, miR-6510-5p, miR-504-3p, miR-3619-3p, miR-671-5p, miR-4667-5p, miR-4430, miR-3195, miR-3679-5p, miR-6076, miR-6515-5p, miR-6820-5p, miR-4634, miR-187-5p, miR-6763-5p, miR-1908-3p, miR-1181, miR-6782-5p, miR-5010-5p, miR-6870-5p, miR-6124, miR-1249-5p, miR-6511b-5p, miR-1254, miR-4727-3p, miR-4259, miR-4771, miR-3622a-5p, miR-4480, miR-4740-5p, miR-6777-5p, miR-6794-5p, miR-4687-3p, miR-6743-5p, miR-6771-5p, miR-3141, miR-3162-5p, miR-4271, miR-1227-5p, miR-4257, miR-4270, miR-4516, miR-4651, miR-4725-3p, miR-6125, miR-6732-5p, miR-6791-5p, miR-6819-5p, miR-6891-5p, miR-7108-5p, miR-7109-5p, miR-642b-3p, and miR-642a-3p, or to a complementary strand(s) of the polynucleotide(s), respectively; and evaluating in vitro whether or not the subject has ovarian tumor using both of the measured expression level(s) and a control expression level(s) from a healthy subject measured in the same way.

(19) A method for detecting ovarian tumor, comprising: measuring an expression level(s) of a target gene(s) in a sample from a subject using a kit according to any of (1) to (7) or a device according to any of (8) to (16), comprising a nucleic acid(s) capable of specifically binding to at least one or at least two polynucleotides selected from the following ovarian tumor markers: miR-4675, miR-4783-3p, miR-1228-5p, miR-4532, miR-6802-5p, miR-6784-5p, miR-3940-5p, miR-1307-3p, miR-8073, miR-3184-5p, miR-1233-5p, miR-6088, miR-5195-3p, miR-320b, miR-4649-5p, miR-6800-5p, miR-1343-3p, miR-4730, miR-6885-5p, miR-5100, miR-1203, miR-6756-5p, miR-373-5p, miR-1268a, miR-1260b, miR-4258, miR-4697-5p, miR-1469, miR-4515, miR-6861-5p, miR-6821-5p, miR-575, miR-6805-5p, miR-4758-5p, miR-3663-3p, miR-4530, miR-6798-5p, miR-6781-5p, miR-885-3p, miR-1273g-3p, miR-4787-3p, miR-4454, miR-4706, miR-1249-3p, miR-887-3p, miR-6786-5p, miR-1238-5p, miR-6749-5p, miR-6729-5p, miR-6825-5p, miR-663b, miR-6858-5p, miR-4690-5p, miR-6765-5p, miR-4710, miR-6775-5p, miR-371a-5p, miR-6816-5p, miR-296-3p, miR-7977, miR-8069, miR-6515-3p, miR-4687-5p, miR-1343-5p, miR-7110-5p, miR-4525, miR-3158-5p, miR-6787-5p, miR-614, miR-4689, miR-1185-2-3p, miR-1268b, miR-1228-3p, miR-1185-1-3p, miR-940, miR-939-5p, miR-6757-5p, miR-1275, miR-5001-5p, miR-6826-5p, miR-6765-3p, miR-3679-3p, miR-4718, miR-4286, miR-8059, miR-4447, miR-4448, miR-658, miR-6766-3p, miR-197-5p, miR-6887-5p, miR-6742-5p, miR-6729-3p, miR-5090, miR-7975, miR-4505, miR-6889-5p, miR-4708-3p, miR-6131, miR-1225-3p, miR-6132, miR-4734, miR-3194-3p, miR-638, miR-2467-3p, miR-4728-5p, miR-5572, miR-6789-5p, miR-8063, miR-4429, miR-6840-3p, miR-4476, miR-675-5p, miR-711, miR-6875-5p, miR-3160-5p, miR-1908-5p, miR-6726-5p, miR-1913, miR-8071, miR-3648, miR-4732-5p, miR-4787-5p, miR-3917, miR-619-5p, miR-1231, miR-342-5p, miR-4433a-5p, miR-6766-5p, miR-4707-5p, miR-7114-5p, miR-6872-3p, miR-6780b-5p, miR-7845-5p, miR-6798-3p, miR-665, miR-6848-5p, miR-5008-5p, miR-4294, miR-6511a-5p, miR-4435, miR-4747-3p, miR-6880-3p, miR-6869-5p, miR-7150, miR-1260a, miR-6877-5p, miR-6721-5p, miR-4656, miR-1229-5p, miR-4433a-3p, miR-4274, miR-4419b, miR-4674, miR-6893-5p, miR-6763-3p, miR-6762-5p, miR-6738-5p, miR-4513, miR-6746-5p, miR-6880-5p, miR-4736, miR-718, miR-6717-5p, miR-7847-3p, miR-760, miR-1199-5p, miR-6813-5p, miR-6769a-5p, miR-1193, miR-7108-3p, miR-6741-5p, miR-4298, miR-6796-3p, miR-4750-5p, miR-6785-5p, miR-1292-3p, miR-4749-3p, miR-6800-3p, miR-4722-5p, miR-4746-3p, miR-4450, miR-6795-5p, miR-365a-5p, miR-498, miR-6797-5p, miR-1470, miR-6851-5p, miR-1247-3p, miR-5196-5p, miR-208a-5p, miR-6842-5p, miR-150-3p, miR-4534, miR-3135b, miR-3131, miR-4792, miR-6510-5p, miR-504-3p, miR-3619-3p, miR-671-5p, miR-4667-5p, miR-4430, miR-3195, miR-3679-5p, miR-6076, miR-6515-5p, miR-6820-5p, miR-4634, miR-187-5p, miR-6763-5p, miR-1908-3p, miR-1181, miR-6782-5p, miR-5010-5p, miR-6870-5p, miR-6124, miR-1249-5p, miR-6511b-5p, miR-1254, miR-4727-3p, miR-4259, miR-4771, miR-3622a-5p, miR-4480, miR-4740-5p, miR-6777-5p, miR-6794-5p, miR-4687-3p, miR-6743-5p, miR-6771-5p, miR-3141, miR-3162-5p, miR-4271, miR-1227-5p, miR-4257, miR-4270, miR-4516, miR-4651, miR-4725-3p, miR-6125, miR-6732-5p, miR-6791-5p, miR-6819-5p, miR-6891-5p, miR-7108-5p, miR-7109-5p, miR-642b-3p, and miR-642a-3p, or to a complementary strand(s) of the polynucleotide(s), respectively; and assigning the expression level(s) of the target gene(s) in the sample from the subject to a discriminant, which is capable of discriminatorily determining the presence or absence of ovarian tumor and is prepared with gene expression levels in a sample(s) from a subject(s) known to have ovarian tumor and a sample(s) from a subject(s) without ovarian tumor as a training sample(s), and thereby evaluating in vitro the presence or absence of ovarian tumor.

(20) The method according to any of (17) to (19), wherein miR-4675 is hsa-miR-4675, miR-4783-3p is hsa-miR-4783-3p, miR-1228-5p is hsa-miR-1228-5p, miR-4532 is hsa-miR-4532, miR-6802-5p is hsa-miR-6802-5p, miR-6784-5p is hsa-miR-6784-5p, miR-3940-5p is hsa-miR-3940-5p, miR-1307-3p is hsa-miR-1307-3p, miR-8073 is hsa-miR-8073, miR-3184-5p is hsa-miR-3184-5p, miR-1233-5p is hsa-miR-1233-5p, miR-6088 is hsa-miR-6088, miR-5195-3p is hsa-miR-5195-3p, miR-320b is hsa-miR-320b, miR-4649-5p is hsa-miR-4649-5p, miR-6800-5p is hsa-miR-6800-5p, miR-1343-3p is hsa-miR-1343-3p, miR-4730 is hsa-miR-4730, miR-6885-5p is hsa-miR-6885-5p, miR-5100 is hsa-miR-5100, miR-1203 is hsa-miR-1203, miR-6756-5p is hsa-miR-6756-5p, miR-373-5p is hsa-miR-373-5p, miR-1268a is hsa-miR-1268a, miR-1260b is hsa-miR-1260b, miR-4258 is hsa-miR-4258, miR-4697-5p is hsa-miR-4697-5p, miR-1469 is hsa-miR-1469, miR-4515 is hsa-miR-4515, miR-6861-5p is hsa-miR-6861-5p, miR-6821-5p is hsa-miR-6821-5p, miR-575 is hsa-miR-575, miR-6805-5p is hsa-miR-6805-5p, miR-4758-5p is hsa-miR-4758-5p, miR-3663-3p is hsa-miR-3663-3p, miR-4530 is hsa-miR-4530, miR-6798-5p is hsa-miR-6798-5p, miR-6781-5p is hsa-miR-6781-5p, miR-885-3p is hsa-miR-885-3p, miR-1273g-3p is hsa-miR-1273g-3p, miR-4787-3p is hsa-miR-4787-3p, miR-4454 is hsa-miR-4454, miR-4706 is hsa-miR-4706, miR-1249-3p is hsa-miR-1249-3p, miR-887-3p is hsa-miR-887-3p, miR-6786-5p is hsa-miR-6786-5p, miR-1238-5p is hsa-miR-1238-5p, miR-6749-5p is hsa-miR-6749-5p, miR-6729-5p is hsa-miR-6729-5p, miR-6825-5p is hsa-miR-6825-5p, miR-663b is hsa-miR-663b, miR-6858-5p is hsa-miR-6858-5p, miR-4690-5p is hsa-miR-4690-5p, miR-6765-5p is hsa-miR-6765-5p, miR-4710 is hsa-miR-4710, miR-6775-5p is hsa-miR-6775-5p, miR-371a-5p is hsa-miR-371a-5p, miR-6816-5p is hsa-miR-6816-5p, miR-296-3p is hsa-miR-296-3p, miR-7977 is hsa-miR-7977, miR-8069 is hsa-miR-8069, miR-6515-3p is hsa-miR-6515-3p, miR-4687-5p is hsa-miR-4687-5p, miR-1343-5p is hsa-miR-1343-5p, miR-7110-5p is hsa-miR-7110-5p, miR-4525 is hsa-miR-4525, miR-3158-5p is hsa-miR-3158-5p, miR-6787-5p is hsa-miR-6787-5p, miR-614 is hsa-miR-614, miR-4689 is hsa-miR-4689, miR-1185-2-3p is hsa-miR-1185-2-3p, miR-1268b is hsa-miR-1268b, miR-1228-3p is hsa-miR-1228-3p, miR-1185-1-3p is hsa-miR-1185-1-3p, miR-940 is hsa-miR-940, miR-939-5p is hsa-miR-939-5p, miR-6757-5p is hsa-miR-6757-5p, miR-1275 is hsa-miR-1275, miR-5001-5p is hsa-miR-5001-5p, miR-6826-5p is hsa-miR-6826-5p, miR-6765-3p is hsa-miR-6765-3p, miR-3679-3p is hsa-miR-3679-3p, miR-4718 is hsa-miR-4718, miR-4286 is hsa-miR-4286, miR-8059 is hsa-miR-8059, miR-4447 is hsa-miR-4447, miR-4448 is hsa-miR-4448, miR-658 is hsa-miR-658, miR-6766-3p is hsa-miR-6766-3p, miR-197-5p is hsa-miR-197-5p, miR-6887-5p is hsa-miR-6887-5p, miR-6742-5p is hsa-miR-6742-5p, miR-6729-3p is hsa-miR-6729-3p, miR-5090 is hsa-miR-5090, miR-7975 is hsa-miR-7975, miR-4505 is hsa-miR-4505, miR-6889-5p is hsa-miR-6889-5p, miR-4708-3p is hsa-miR-4708-3p, miR-6131 is hsa-miR-6131, miR-1225-3p is hsa-miR-1225-3p, miR-6132 is hsa-miR-6132, miR-4734 is hsa-miR-4734, miR-3194-3p is hsa-miR-3194-3p, miR-638 is hsa-miR-638, miR-2467-3p is hsa-miR-2467-3p, miR-4728-5p is hsa-miR-4728-5p, miR-5572 is hsa-miR-5572, miR-6789-5p is hsa-miR-6789-5p, miR-8063 is hsa-miR-8063, miR-4429 is hsa-miR-4429, miR-6840-3p is hsa-miR-6840-3p, miR-4476 is hsa-miR-4476, miR-675-5p is hsa-miR-675-5p, miR-711 is hsa-miR-711, miR-6875-5p is hsa-miR-6875-5p, miR-3160-5p is hsa-miR-3160-5p, miR-1908-5p is hsa-miR-1908-5p, miR-6726-5p is hsa-miR-6726-5p, miR-1913 is hsa-miR-1913, miR-8071 is hsa-miR-8071, miR-3648 is hsa-miR-3648, miR-4732-5p is hsa-miR-4732-5p, miR-4787-5p is hsa-miR-4787-5p, miR-3917 is hsa-miR-3917, miR-619-5p is hsa-miR-619-5p, miR-1231 is hsa-miR-1231, miR-342-5p is hsa-miR-342-5p, miR-4433a-5p is hsa-miR-4433a-5p, miR-6766-5p is hsa-miR-6766-5p, miR-4707-5p is hsa-miR-4707-5p, miR-7114-5p is hsa-miR-7114-5p, miR-6872-3p is hsa-miR-6872-3p, miR-6780b-5p is hsa-miR-6780b-5p, miR-7845-5p is hsa-miR-7845-5p, miR-6798-3p is hsa-miR-6798-3p, miR-665 is hsa-miR-665, miR-6848-5p is hsa-miR-6848-5p, miR-5008-5p is hsa-miR-5008-5p, miR-4294 is hsa-miR-4294, miR-6511a-5p is hsa-miR-6511a-5p, miR-4435 is hsa-miR-4435, miR-4747-3p is hsa-miR-4747-3p, miR-6880-3p is hsa-miR-6880-3p, miR-6869-5p is hsa-miR-6869-5p, miR-7150 is hsa-miR-7150, miR-1260a is hsa-miR-1260a, miR-6877-5p is hsa-miR-6877-5p, miR-6721-5p is hsa-miR-6721-5p, miR-4656 is hsa-miR-4656, miR-1229-5p is hsa-miR-1229-5p, miR-4433a-3p is hsa-miR-4433a-3p, miR-4274 is hsa-miR-4274, miR-4419b is hsa-miR-4419b, miR-4674 is hsa-miR-4674, miR-6893-5p is hsa-miR-6893-5p, miR-6763-3p is hsa-miR-6763-3p, miR-6762-5p is hsa-miR-6762-5p, miR-6738-5p is hsa-miR-6738-5p, miR-4513 is hsa-miR-4513, miR-6746-5p is hsa-miR-6746-5p, miR-6880-5p is hsa-miR-6880-5p, miR-4736 is hsa-miR-4736, miR-718 is hsa-miR-718, miR-6717-5p is hsa-miR-6717-5p, miR-7847-3p is hsa-miR-7847-3p, miR-760 is hsa-miR-760, miR-1199-5p is hsa-miR-1199-5p, miR-6813-5p is hsa-miR-6813-5p, miR-6769a-5p is hsa-miR-6769a-5p, miR-1193 is hsa-miR-1193, miR-7108-3p is hsa-miR-7108-3p, miR-6741-5p is hsa-miR-6741-5p, miR-4298 is hsa-miR-4298, miR-6796-3p is hsa-miR-6796-3p, miR-4750-5p is hsa-miR-4750-5p, miR-6785-5p is hsa-miR-6785-5p, miR-1292-3p is hsa-miR-1292-3p, miR-4749-3p is hsa-miR-4749-3p, miR-6800-3p is hsa-miR-6800-3p, miR-722-5p is hsa-miR-4722-5p, miR-4746-3p is hsa-miR-4746-3p, miR-4450 is hsa-miR-4450, miR-6795-5p is hsa-miR-6795-5p, miR-365a-5p is hsa-miR-365a-5p, miR-498 is hsa-miR-498, miR-6797-5p is hsa-miR-6797-5p, miR-1470 is hsa-miR-1470, miR-6851-5p is hsa-miR-6851-5p, miR-1247-3p is hsa-miR-1247-3p, miR-5196-5p is hsa-miR-5196-5p, miR-208a-5p is hsa-miR-208a-5p, miR-6842-5p is hsa-miR-6842-5p, miR-150-3p is hsa-miR-150-3p, miR-4534 is hsa-miR-4534, miR-3135b is hsa-miR-3135b, miR-3131 is hsa-miR-3131, miR-4792 is hsa-miR-4792, miR-6510-5p is hsa-miR-6510-5p, miR-504-3p is hsa-miR-504-3p, miR-3619-3p is hsa-miR-3619-3p, miR-671-5p is hsa-miR-671-5p, miR-4667-5p is hsa-miR-4667-5p, miR-4430 is hsa-miR-4430, miR-3195 is hsa-miR-3195, miR-3679-5p is hsa-miR-3679-5p, miR-6076 is hsa-miR-6076, miR-6515-5p is hsa-miR-6515-5p, miR-6820-5p is hsa-miR-6820-5p, miR-4634 is hsa-miR-4634, miR-187-5p is hsa-miR-187-5p, miR-6763-5p is hsa-miR-6763-5p, miR-1908-3p is hsa-miR-1908-3p, miR-1181 is hsa-miR-1181, miR-6782-5p is hsa-miR-6782-5p, miR-5010-5p is hsa-miR-5010-5p, miR-6870-5p is hsa-miR-6870-5p, miR-6124 is hsa-miR-6124, miR-1249-5p is hsa-miR-1249-5p, miR-6511b-5p is hsa-miR-6511b-5p, miR-1254 is hsa-miR-1254, miR-4727-3p is hsa-miR-4727-3p, miR-4259 is hsa-miR-4259, miR-4771 is hsa-miR-4771, miR-3622a-5p is hsa-miR-3622a-5p, miR-4480 is hsa-miR-4480, miR-4740-5p is hsa-miR-4740-5p, miR-6777-5p is hsa-miR-6777-5p, miR-6794-5p is hsa-miR-6794-5p, miR-4687-3p is hsa-miR-4687-3p, miR-6743-5p is hsa-miR-6743-5p, miR-6771-5p is hsa-miR-6771-5p, miR-3141 is hsa-miR-3141, miR-3162-5p is hsa-miR-3162-5p, miR-4271 is hsa-miR-4271, miR-1227-5p is hsa-miR-1227-5p, miR-4257 is hsa-miR-4257, miR-4270 is hsa-miR-4270, miR-4516 is hsa-miR-4516, miR-4651 is hsa-miR-4651, miR-4725-3p is hsa-miR-4725-3p, miR-6125 is hsa-miR-6125, miR-6732-5p is hsa-miR-6732-5p, miR-6791-5p is hsa-miR-6791-5p, miR-6819-5p is hsa-miR-6819-5p, miR-6891-5p is hsa-miR-6891-5p, miR-7108-5p is hsa-miR-7108-5p, miR-7109-5p is hsa-miR-7109-5p, miR-642b-3p is hsa-miR-642b-3p, and miR-642a-3p is hsa-miR-642a-3p.

(21) The method according to any of (17) to (19), wherein the nucleic acid(s) is a polynucleotide(s) selected from the group consisting of the following polynucleotides (a) to (e):
(a) a polynucleotide consisting of a nucleotide sequence represented by any of SEQ ID NOs: 1 to 247, 251, and 268 or a nucleotide sequence derived from the nucleotide sequence by the replacement of u with t, a variant thereof, a derivative thereof, or a fragment thereof comprising 15 or more consecutive nucleotides;
(b) a polynucleotide comprising a nucleotide sequence represented by any of SEQ ID NOs: 1 to 247, 251, and 268;
(c) a polynucleotide consisting of a nucleotide sequence complementary to a nucleotide sequence represented by any of SEQ ID NOs: 1 to 247, 251, and 268 or a nucleotide sequence derived from the nucleotide sequence by the replacement of u with t, a variant thereof, a derivative thereof, or a fragment thereof comprising 15 or more consecutive nucleotides;
(d) a polynucleotide comprising a nucleotide sequence complementary to a nucleotide sequence represented by any of SEQ ID NOs: 1 to 247, 251, and 268 or a nucleotide sequence derived from the nucleotide sequence by the replacement of u with t; and
(e) a polynucleotide hybridizing under stringent conditions to any of the polynucleotides (a) to (d).

(22) The method according to any of (17) to (21), wherein the kit or device further comprises a nucleic acid(s) capable of specifically binding to at least one polynucleotide selected from the following other ovarian tumor markers: miR-320a, miR-663a, miR-328-5p, miR-128-2-5p, miR-125a-3p, miR-191-5p, miR-92b-5p, miR-296-5p, miR-1246, miR-92a-2-5p, miR-128-1-5p, miR-1290, miR-211-3p, miR-744-5p, miR-135a-3p, miR-451a, miR-625-3p, miR-92a-3p, miR-422a, miR-483-5p, miR-652-5p, miR-24-3p, miR-23b-3p, miR-23a-3p, miR-92b-3p, and miR-22-3p, or to a complementary strand of the polynucleotide.

(23) The method according to (22), wherein miR-320a is hsa-miR-320a, miR-663a is hsa-miR-663a, miR-328-5p is hsa-miR-328-5p, miR-128-2-5p is hsa-miR-128-2-5p, miR-125a-3p is hsa-miR-125a-3p, miR-191-5p is hsa-miR-191-5p, miR-92b-5p is hsa-miR-92b-5p, miR-296-5p is hsa-miR-296-5p, miR-1246 is hsa-miR-1246, miR-92a-2-5p is hsa-miR-92a-2-5p, miR-128-1-5p is hsa-miR-128-1-5p, miR-1290 is hsa-miR-1290, miR-211-3p is hsa-miR-211-3p, miR-744-5p is hsa-miR-744-5p, miR-135a-3p is hsa-miR-135a-3p, miR-451a is hsa-miR-451a, miR-625-3p is hsa-miR-625-3p, miR-92a-3p is hsa-miR-92a-3p, miR-422a is hsa-miR-422a, miR-483-5p is hsa-miR-483-5p, miR-652-5p is hsa-miR-652-5p, miR-24-3p is hsa-miR-24-3p, miR-23b-3p is hsa-miR-23b-3p, miR-23a-3p is hsa-miR-23a-3p, miR-92b-3p is hsa-miR-92b-3p, and miR-22-3p is hsa-miR-22-3p.

(24) The method according to (22) or (23), wherein the nucleic acid(s) is a polynucleotide(s) selected from the group consisting of the following polynucleotides (f) to (j):
(f) a polynucleotide consisting of a nucleotide sequence represented by any of SEQ ID NOs: 248 to 250, 252 to 267, and 269 to 275 or a nucleotide sequence derived from the nucleotide sequence by the replacement of u with t, a variant thereof, a derivative thereof, or a fragment thereof comprising 15 or more consecutive nucleotides;
(g) a polynucleotide comprising a nucleotide sequence represented by any of SEQ ID NOs: 248 to 250, 252 to 267, and 269 to 275;
(h) a polynucleotide consisting of a nucleotide sequence complementary to a nucleotide sequence represented by any of SEQ ID NOs: 248 to 250, 252 to 267, and 269 to 275 or a nucleotide sequence derived from the nucleotide sequence by the replacement of u with t, a variant thereof, a derivative thereof, or a fragment thereof comprising 15 or more consecutive nucleotides;
(i) a polynucleotide comprising a nucleotide sequence complementary to a nucleotide sequence represented by any of SEQ ID NOs: 248 to 250, 252 to 267, and 269 to 275 or a nucleotide sequence derived from the nucleotide sequence by the replacement of u with t; and
j) a polynucleotide hybridizing under stringent conditions to any of the polynucleotides (f) to (i).

(25) The method according to any one of (17) to (24), wherein the subject is a human.

(26) The method according to any one of (17) to (25), wherein the sample is blood, serum, or plasma.

### DEFINITION OF TERMS

The terms used herein are defined as described below.

Abbreviations or terms such as nucleotide, polynucleotide, DNA, and RNA abide by “Guidelines for the preparation of specification which contain nucleotide and/or amino acid sequences” (edited by Japan Patent Office) and common use in the art.

The term “polynucleotide” used herein refers to a nucleic acid including any of RNA, DNA, and RNA/DNA (chimera). The DNA includes any of cDNA, genomic DNA, and synthetic DNA. The RNA includes any of total RNA, mRNA, rRNA, miRNA, siRNA, snoRNA, snRNA, non-coding RNA and synthetic RNA. Here the “synthetic DNA” and the “synthetic RNA” refer to a DNA and an RNA artificially prepared using, for example, an automatic nucleic acid synthesizer, on the basis of predetermined nucleotide sequences (which may be any of natural and non-natural sequences). The “non-natural sequence” is intended to be used in a broad sense and includes, for example, a sequence comprising substitution, deletion, insertion, and/or addition of one or more nucleotides (i.e., a variant sequence) and a sequence comprising one or more modified nucleotides (i.e., a modified sequence), which are different from the natural sequence. Herein, the term “polynucleotide” is used interchangeably with the term “nucleic acid.”

The term “fragment” used herein is a polynucleotide having a nucleotide sequence that consists of a consecutive portion of a polynucleotide and desirably has a length of 15 or more nucleotides, preferably 17 or more nucleotides, more preferably 19 or more nucleotides.

The term “gene” used herein is intended to include not only RNA and double-stranded DNA but also each single-stranded DNA such as a plus(+) strand (or a sense strand) or a complementary strand (or an antisense strand) constituting the duplex. The gene is not particularly limited by its length. Thus, the “gene” used herein includes any of double-stranded DNA including human genomic DNA, single-stranded DNA (plus strand), single-stranded DNA having a sequence complementary to the plus strand (complementary strand), cDNA, microRNA (miRNA), their fragments, and human genome, and their transcripts, unless otherwise specified. The “gene” includes not only a “gene” represented by a particular nucleotide sequence (or SEQ ID NO) but “nucleic acids” encoding RNAs having biological functions equivalent to RNA encoded by the gene, for example, a congener (i.e., a homolog or an ortholog), a variant (e.g., a genetic polymorph), and a derivative. Specific examples of such a “nucleic acid” encoding a congener, a variant, or a derivative can include a “nucleic acid” having a nucleotide sequence hybridizing under stringent conditions described later to a complementary sequence of a nucleotide sequence represented by any of SEQ ID NOs: 1 to 842 or a nucleotide sequence derived from the nucleotide sequence by the replacement of u with t. Regardless whether or not there is a difference in functional region, the “gene” can comprise, for example, expression control regions, coding region, exons, or introns. The “gene” may be contained in a cell or may exist alone after being released from a cell. Alternatively, the “gene” may be in a state enclosed in a vesicle called exosome.

The term “exosome” used herein is a vesicle that is encapsulated by lipid bilayer and secreted from a cell. The exosome is derived from a multivesicular endosome and may incorporate biomaterials such as “genes” (e.g., RNA or DNA) or proteins when released into an extracellular environment. The exosome is known to be contained in a body fluid such as blood, serum, plasma, serum or lymph.

The term “transcript” used herein refers to an RNA synthesized from the DNA sequence of a gene as a template. RNA polymerase binds to a site called promoter located upstream of the gene and adds ribonucleotides complementary to the nucleotide sequence of the DNA to the 3′ end to synthesize an RNA. This RNA contains not only the gene itself but the whole sequence from a transcription initiation site to the end of a polyA sequence, including expression control regions, coding region, exons, or introns.

Unless otherwise specified, the term “microRNA (miRNA)” used herein is intended to mean a 15- to 25-nucleotide non-coding RNA that is transcribed as an RNA precursor having a hairpin-like structure, cleaved by a dsRNA-cleaving enzyme having RNase III cleavage activity, and integrated into a protein complex called RISC, and that is involved in the suppression of translation of mRNA. The term “miRNA” used herein includes not only a “miRNA” represented by a particular nucleotide sequence (or SEQ ID NO) but a “miRNA” comprising a precursor of the “miRNA” (pre-miRNA or pri-miRNA) and having biological functions equivalent to miRNAs encoded by these, for example, a “miRNA” encoding a congener (i.e., a homolog or an ortholog), a variant such as a genetic polymorph, and a derivative. Such a “miRNA” encoding a precursor, a congener, a variant, or a derivative can be specifically identified using miRBase Release 21 (http://www.mirbase.org/), and examples thereof can include a “miRNA” having a nucleotide sequence hybridizing under stringent conditions described later to a complementary sequence of any particular nucleotide sequence represented by any of SEQ ID NOs: 1 to 842. The term “miRNA” used herein may be a gene product of a miR gene. Such a gene product includes a mature miRNA (e.g., a 15- to 25-nucleotide or 19- to 25-nucleotide non-coding RNA involved in the suppression of translation of mRNA as described above) or a miRNA precursor (e.g., pre-miRNA or pri-miRNA as described above).

The term “probe” used herein includes a polynucleotide that is used for specifically detecting an RNA resulting from the expression of a gene or a polynucleotide derived from the RNA, and/or a polynucleotide complementary thereto.

The term “primer” used herein includes consecutive polynucleotides that specifically recognize and amplify an RNA resulting from the expression of a gene or a polynucleotide derived from the RNA, and/or a polynucleotide complementary thereto.

In this context, the complementary polynucleotide (complementary strand or reverse strand) means a polynucleotide in a complementary relationship based on A:T (U) and G:C base pairs with the full-length sequence of a polynucleotide consisting of a nucleotide sequence defined by any of SEQ ID NOs: 1 to 842 or a nucleotide sequence derived from the nucleotide sequence by the replacement of u with t, or a partial sequence thereof (here, this full-length or partial sequence is referred to as a plus strand for the sake of convenience). However, such a complementary strand is not limited to a sequence completely complementary to the nucleotide sequence of the target plus strand and may have a complementary relationship to an extent that permits hybridization under stringent conditions to the target plus strand.

The term “stringent conditions” used herein refers to conditions under which a nucleic acid probe hybridizes to its target sequence to a detectably larger extent (e.g., a measurement value equal to or larger than “(a mean of background measurement values)+(a standard deviation of the background measurement values)×2”) than that for other sequences. The stringent conditions are dependent on a sequence and differ depending on an environment where hybridization is performed. A target sequence complementary 100% to the nucleic acid probe can be identified by controlling the stringency of hybridization and/or washing conditions. Specific examples of the “stringent conditions” will be mentioned later.

The term “Tm value” used herein means a temperature at which the double-stranded moiety of a polynucleotide is denatured into single strands so that the double strands and the single strands exist at a ratio of 1:1.

The term “variant” used herein means, in the case of a nucleic acid, a natural variant attributed to polymorphism, mutation, or the like; a variant containing the deletion, substitution, addition, or insertion of 1, 2 or 3 or more (e.g., 1 to several) nucleotides in a nucleotide sequence represented by a SEQ ID NO or a nucleotide sequence derived from the nucleotide sequence by the replacement of u with t, or a partial sequence thereof; a variant containing the deletion, substitution, addition, or insertion of 1 or 2 or more nucleotides in a nucleotide sequence of a premature miRNA of the sequence of any of SEQ ID NOs 1 to 275 or a nucleotide sequence derived from the nucleotide sequence by the replacement of u with t, or a partial sequence thereof; a variant that exhibits percent (%) identity of approximately 90% or higher, approximately 95% or higher, approximately 97% or higher, approximately 98% or higher, approximately 99% or higher to each of these nucleotide sequences or the partial sequences thereof; or a nucleic acid hybridizing under the stringent conditions defined above to a polynucleotide or an oligonucleotide comprising each of these nucleotide sequences or the partial sequences thereof.

The term “several” used herein means an integer of approximately 10, 9, 8, 7, 6, 5, 4, 3, or 2.

The variant as used herein can be prepared by use of a well-known technique such as site-directed mutagenesis or mutagenesis using PCR.

The term “percent (%) identity” used herein can be determined with or without an introduced gap, using a protein or gene search system based on BLAST (https://blast.ncbi.nlm.nih.gov/Blast.cgi) or FASTA (http://www.genome.jp/tools/fasta/) (Zheng Zhang et al., 2000, J. Comput. Biol., Vol. 7, p. 203-214; Altschul, S. F. et al., 1990, Journal of Molecular Biology, Vol. 215, p. 403-410; and Pearson, W. R. et al., 1988, Proc. Natl. Acad. Sci. U.S.A, Vol. 85, p. 2444-2448).

The term “derivative” used herein is meant to include unlimitedly a modified nucleic acid, for example, a derivative labeled with a fluorophore or the like, a derivative containing a modified nucleotide (e.g., a nucleotide containing a group such as halogen, alkyl such as methyl, alkoxy such as methoxy, thio, or carboxymethyl, and a nucleotide that has undergone base rearrangement, double bond saturation, deamination, replacement of an oxygen molecule with a sulfur atom, etc.), PNA (peptide nucleic acid; Nielsen, P. E. et al., 1991, Science, Vol. 254, p. 1497-500), and LNA (locked nucleic acid; Obika, S. et al., 1998, Tetrahedron Lett., Vol. 39, p. 5401-5404).

As used herein, the “nucleic acid” capable of specifically binding to a polynucleotide selected from the ovarian tumor marker miRNAs described above or to a complementary strand of the polynucleotide, or the “nucleic acid” capable of detecting the polynucleotide, is a synthesized or prepared nucleic acid and, for example, includes a “nucleic acid probe” or a “primer”, and is utilized directly or indirectly for detecting the presence or absence of ovarian tumor in a subject, for diagnosing the presence or absence or the severity of ovarian tumor, the presence or absence or the degree of amelioration of ovarian tumor, or the therapeutic sensitivity of ovarian tumor, or for screening for a candidate substance useful in the prevention, amelioration, or treatment of ovarian tumor. The “nucleic acid” includes a nucleotide, an oligonucleotide, and a polynucleotide capable of specifically recognizing and binding to a transcript represented by any of SEQ ID NOs: 1 to 842 or a synthetic cDNA nucleic acid thereof in vivo, particularly, in a sample such as a body fluid (e.g., blood or urine), in relation to the development of ovarian tumor. The nucleotide, the oligonucleotide, and the polynucleotide can be effectively used as probes for detecting the aforementioned gene expressed in vivo, in tissues, in cells, or the like on the basis of the properties described above, or as primers for amplifying the aforementioned gene expressed in vivo.

The term “detection” used herein is interchangeable with the term “examination”, “measurement”, “detection”, or “decision support”. As used herein, the term “evaluation” is meant to include diagnosing- or evaluation-supporting on the basis of examination results or measurement results.

The term “subject” used herein means a mammal such as a primate including a human and a chimpanzee, a pet animal including a dog and a cat, a livestock animal including cattle, a horse, sheep, and a goat, a rodent including a mouse and a rat, and animals raised in a zoo. The subject is preferably a human. The term “subject” herein may be optionally referred to as “test subject”. The term “healthy subject” also means such a mammal, which is an animal or a subject without the cancer to be detected. The healthy subject is preferably a human.

The term “ovarian cancer” used herein is a malignant tumor which develops in the ovary and includes, for example, epithelial ovarian cancer which develops from the mucosal epithelium, and germ cell stromal ovarian cancers.

The term “ovarian benign tumor” used herein is a benign tumor which develops in the ovary and includes, for example, mucinous cystadenoma, serous cystadenoma, mature teratoma and fibroma.

The term “ovarian tumor” used herein is a term including the above “ovarian cancers” and “ovarian benign tumors”.

The term “P” or “P value” used herein refers to a probability at which a more extreme statistic than that actually calculated from data under null hypothesis is observed in a statistical test. Thus, smaller “P” or “P value” is regarded as being a more significant difference between subjects to be compared.

The term “sensitivity” used herein means a value of (the number of true positives)/(the number of true positives+the number of false negatives). High sensitivity allows ovarian tumor to be detected early, leading to the complete resection of cancer sites and reduction in the rate of recurrence.

The term “specificity” used herein means a value of (the number of true negatives)/(the number of true negatives+the number of false positives). High specificity prevents needless extra examination for healthy subjects misjudged as being ovarian tumor patients, leading to reduction in burden on patients and reduction in medical expense.

The term “accuracy” used herein means a value of (the number of true positives+the number of true negatives)/(the total number of cases). The accuracy indicates the ratio of samples that are identified correctly to all samples, and serves as a primary index for evaluating detection performance.

As used herein, the “sample” that is subjected to determination, detection, or diagnosis refers to a tissue and a biological material in which the expression of the gene of the present invention varies as ovarian tumor develops, as ovarian tumor progresses, or as therapeutic effects on ovarian tumor are exerted. Specifically, the sample refers to an ovarian tissue and fallopian tube, lymph node and a surrounding organ thereof, an organ suspected of having metastasis, the skin, a body fluid such as blood, urine, saliva, sweat, or tissue exudates, serum or plasma prepared from blood, feces, hair, and the like. The “sample” further refers to a biological sample extracted therefrom, specifically, a gene such as RNA or miRNA.

The term “hsa-miR-4675 gene” or “hsa-miR-4675” used herein includes the hsa-miR-4675 gene (miRBase Accession No. MIMAT0019757) described in SEQ ID NO: 1, a homolog or an ortholog of a different organism species, and the like. The hsa-miR-4675 gene can be obtained by a method described in Persson H et al., 2011, Cancer Res, Vol. 71, p. 78-86. Also, “hsa-mir-4675” (miRBase Accession No. M10017306, SEQ ID NO: 276) having a hairpin-like structure is known as a precursor of “hsa-miR-4675”.

The term “hsa-miR-4783-3p gene” or “hsa-miR-4783-3p” used herein includes the hsa-miR-4783-3p gene (miRBase Accession No. MIMAT0019947) described in SEQ ID NO: 2, a homolog or an ortholog of a different organism species, and the like. The hsa-miR-4783-3p gene can be obtained by a method described in Persson H et al., 2011, Cancer Res, Vol. 71, p. 78-86. Also, “hsa-mir-4783” (miRBase Accession No. MI0017428, SEQ ID NO: 277) having a hairpin-like structure is known as a precursor of “hsa-miR-4783-3p”.

The term “hsa-miR-1228-5p gene” or “hsa-miR-1228-5p” used herein includes the hsa-miR-1228-5p gene (miRBase Accession No. MIMAT0005582) described in SEQ ID NO: 3, a homolog or an ortholog of a different organism species, and the like. The hsa-miR-1228-5p gene can be obtained by a method described in Berezikov E et al., 2007, Mol Cell, Vol. 28, p. 328-336. Also, “hsa-mir-1228” (miRBase Accession No. MI0006318, SEQ ID NO: 278) having a hairpin-like structure is known as a precursor of “hsa-miR-1228-5p”.

The term “hsa-miR-4532 gene” or “hsa-miR-4532” used herein includes the hsa-miR-4532 gene (miRBase Accession No. MIMAT0019071) described in SEQ ID NO: 4, a homolog or an ortholog of a different organism species, and the like. The hsa-miR-4532 gene can be obtained by a method described in Jima D D et al., 2010, Blood, Vol. 116, e118-e127. Also, “hsa-mir-4532” (miRBase Accession No. MI0016899, SEQ ID NO: 279) having a hairpin-like structure is known as a precursor of “hsa-miR-4532”.

The term “hsa-miR-6802-5p gene” or “hsa-miR-6802-5p” used herein includes the hsa-miR-6802-5p gene (miRBase Accession No. MIMAT0027504) described in SEQ ID NO: 5, a homolog or an ortholog of a different organism species, and the like. The hsa-miR-6802-5p gene can be obtained by a method described in Ladewig E et al., 2012, Genome Res, Vol. 22, p. 1634-1645. Also, “hsa-mir-6802” (miRBase Accession No. MI0022647, SEQ ID NO: 280) having a hairpin-like structure is known as a precursor of “hsa-miR-6802-5p”.

The term “hsa-miR-6784-5p gene” or “hsa-miR-6784-5p” used herein includes the hsa-miR-6784-5p gene (miRBase Accession No. MIMAT0027468) described in SEQ ID NO: 6, a homolog or an ortholog of a different organism species, and the like. The hsa-miR-6784-5p gene can be obtained by a method described in Ladewig E et al., 2012, Genome Res, Vol. 22, p. 1634-1645. Also, “hsa-mir-6784” (miRBase Accession No. MI0022629, SEQ ID NO: 281) having a hairpin-like structure is known as a precursor of “hsa-miR-6784-5p”.

The term “hsa-miR-3940-5p gene” or “hsa-miR-3940-5p” used herein includes the hsa-miR-3940-5p gene (miRBase Accession No. MIMAT0019229) described in SEQ ID NO: 7, a homolog or an ortholog of a different organism species, and the like. The hsa-miR-3940-5p gene can be obtained by a method described in Liao J Y et al., 2010, PLoS One, Vol. 5, e10563. Also, “hsa-mir-3940” (miRBase Accession No. MI0016597, SEQ ID NO: 282) having a hairpin-like structure is known as a precursor of “hsa-miR-3940-5p”.

The term “hsa-miR-1307-3p gene” or “hsa-miR-1307-3p” used herein includes the hsa-miR-1307-3p gene (miRBase Accession No. MIMAT0005951) described in SEQ ID NO: 8, a homolog or an ortholog of a different organism species, and the like. The hsa-miR-1307-3p gene can be obtained by a method described in Morin R D et al., 2008, Genome Res, Vol. 18, p. 610-621. Also, “hsa-mir-1307” (miRBase Accession No. MI0006444, SEQ ID NO: 283) having a hairpin-like structure is known as a precursor of “hsa-miR-1307-3p”.

The term “hsa-miR-8073 gene” or “hsa-miR-8073” used herein includes the hsa-miR-8073 gene (miRBase Accession No. MIMAT0031000) described in SEQ ID NO: 9, a homolog or an ortholog of a different organism species, and the like. The hsa-miR-8073 gene can be obtained by a method described in Wang H J et al., 2013, Shock, Vol. 39, p. 480-487. Also, “hsa-mir-8073” (miRBase Accession No. MI0025909, SEQ ID NO: 284) having a hairpin-like structure is known as a precursor of “hsa-miR-8073”.

The term “hsa-miR-3184-5p gene” or “hsa-miR-3184-5p” used herein includes the hsa-miR-3184-5p gene (miRBase Accession No. MIMAT0015064) described in SEQ ID NO: 10, a homolog or an ortholog of a different organism species, and the like. The hsa-miR-3184-5p gene can be obtained by a method described in Stark M S et al., 2010, PLoS One, Vol. 5, e9685. Also, “hsa-mir-3184” (miRBase Accession No. MI0014226, SEQ ID NO: 285) having a hairpin-like structure is known as a precursor of “hsa-miR-3184-5p”.

The term “hsa-miR-1233-5p gene” or “hsa-miR-1233-5p” used herein includes the hsa-miR-1233-5p gene (miRBase Accession No. MIMAT0022943) described in SEQ ID NO: 11, a homolog or an ortholog of a different organism species, and the like. The hsa-miR-1233-5p gene can be obtained by a method described in Berezikov E et al., 2007, Mol Cell, Vol. 28, p. 328-336. Also, “hsa-mir-1233-1 and hsa-mir-1233-2” (miRBase Accession Nos. MI0006323 and MI0015973, SEQ ID NOs: 286 and 287) having a hairpin-like structure are known as precursors of “hsa-miR-1233-5p”.

The term “hsa-miR-6088 gene” or “hsa-miR-6088” used herein includes the hsa-miR-6088 gene (miRBase Accession No. MIMAT0023713) described in SEQ ID NO: 12, a homolog or an ortholog of a different organism species, and the like. The hsa-miR-6088 gene can be obtained by a method described in Yoo J K et al., 2012, Stem Cells Dev, Vol. 21, p. 2049-2057. Also, “hsa-mir-6088” (miRBase Accession No. MI0020365, SEQ ID NO: 288) having a hairpin-like structure is known as a precursor of “hsa-miR-6088”.

The term “hsa-miR-5195-3p gene” or “hsa-miR-5195-3p” used herein includes the hsa-miR-5195-3p gene (miRBase Accession No. MIMAT0021127) described in SEQ ID NO: 13, a homolog or an ortholog of a different organism species, and the like. The hsa-miR-5195-3p gene can be obtained by a method described in Schotte D et al., 2011, Leukemia, Vol. 25, p. 1389-1399. Also, “hsa-mir-5195” (miRBase Accession No. MI0018174, SEQ ID NO: 289) having a hairpin-like structure is known as a precursor of “hsa-miR-5195-3p”.

The term “hsa-miR-320b gene” or “hsa-miR-320b” used herein includes the hsa-miR-320b gene (miRBase Accession No. MIMAT0005792) described in SEQ ID NO: 14, a homolog or an ortholog of a different organism species, and the like. The hsa-miR-320b gene can be obtained by a method described in Berezikov E et al., 2006, Genome Res, Vol. 16, p. 1289-1298. Also, “hsa-mir-320b-1 and hsa-mir-320b-2” (miRBase Accession Nos. MI0003776 and MI0003839, SEQ ID NOs: 290 and 291) having a hairpin-like structure are known as precursors of “hsa-miR-320b”.

The term “hsa-miR-4649-5p gene” or “hsa-miR-4649-5p” used herein includes the hsa-miR-4649-5p gene (miRBase Accession No. MIMAT0019711) described in SEQ ID NO: 15, a homolog or an ortholog of a different organism species, and the like. The hsa-miR-4649-5p gene can be obtained by a method described in Persson H et al., 2011, Cancer Res, Vol. 71, p. 78-86. Also, “hsa-mir-4649” (miRBase Accession No. MI0017276, SEQ ID NO: 292) having a hairpin-like structure is known as a precursor of “hsa-miR-4649-5p”.

The term “hsa-miR-6800-5p gene” or “hsa-miR-6800-5p” used herein includes the hsa-miR-6800-5p gene (miRBase Accession No. MIMAT0027500) described in SEQ ID NO: 16, a homolog or an ortholog of a different organism species, and the like. The hsa-miR-6800-5p gene can be obtained by a method described in Ladewig E et al., 2012, Genome Res, Vol. 22, p. 1634-1645. Also, “hsa-mir-6800” (miRBase Accession No. MI0022645, SEQ ID NO: 293) having a hairpin-like structure is known as a precursor of “hsa-miR-6800-5p”.

The term “hsa-miR-1343-3p gene” or “hsa-miR-1343-3p” used herein includes the hsa-miR-1343-3p gene (miRBase Accession No. MIMAT0019776) described in SEQ ID NO: 17, a homolog or an ortholog of a different organism species, and the like. The hsa-miR-1343-3p gene can be obtained by a method described in Persson H et al., 2011, Cancer Res, Vol. 71, p. 78-86. Also, “hsa-mir-1343” (miRBase Accession No. MI0017320, SEQ ID NO: 294) having a hairpin-like structure is known as a precursor of “hsa-miR-1343-3p”.

The term “hsa-miR-4730 gene” or “hsa-miR-4730” used herein includes the hsa-miR-4730 gene (miRBase Accession No. MIMAT0019852) described in SEQ ID NO: 18, a homolog or an ortholog of a different organism species, and the like. The hsa-miR-4730 gene can be obtained by a method described in Persson H et al., 2011, Cancer Res, Vol. 71, p. 78-86. Also, “hsa-mir-4730” (miRBase Accession No. M10017367, SEQ ID NO: 295) having a hairpin-like structure is known as a precursor of “hsa-miR-4730”.

The term “hsa-miR-6885-5p gene” or “hsa-miR-6885-5p” used herein includes the hsa-miR-6885-5p gene (miRBase Accession No. MIMAT0027670) described in SEQ ID NO: 19, a homolog or an ortholog of a different organism species, and the like. The hsa-miR-6885-5p gene can be obtained by a method described in Ladewig E et al., 2012, Genome Res, Vol. 22, p. 1634-1645. Also, “hsa-mir-6885” (miRBase Accession No. MI0022732, SEQ ID NO: 296) having a hairpin-like structure is known as a precursor of “hsa-miR-6885-5p”.

The term “hsa-miR-5100 gene” or “hsa-miR-5100” used herein includes the hsa-miR-5100 gene (miRBase Accession No. MIMAT0022259) described in SEQ ID NO: 20, a homolog or an ortholog of a different organism species, and the like. The hsa-miR-5100 gene can be obtained by a method described in Tandon M et al., 2012, Oral Dis, Vol. 18, p. 127-131. Also, “hsa-mir-5100” (miRBase Accession No. MI0019116, SEQ ID NO: 297) having a hairpin-like structure is known as a precursor of “hsa-miR-5100”.

The term “hsa-miR-1203 gene” or “hsa-miR-1203” used herein includes the hsa-miR-1203 gene (miRBase Accession No. MIMAT0005866) described in SEQ ID NO: 21, a homolog or an ortholog of a different organism species, and the like. The hsa-miR-1203 gene can be obtained by a method described in Marton S et al., 2008, Leukemia, Vol. 22, p. 330-338. Also, “hsa-mir-1203” (miRBase Accession No. MI0006335, SEQ ID NO: 298) having a hairpin-like structure is known as a precursor of “hsa-miR-1203”.

The term “hsa-miR-6756-5p gene” or “hsa-miR-6756-5p” used herein includes the hsa-miR-6756-5p gene (miRBase Accession No. MIMAT0027412) described in SEQ ID NO: 22, a homolog or an ortholog of a different organism species, and the like. The hsa-miR-6756-5p gene can be obtained by a method described in Ladewig E et al., 2012, Genome Res, Vol. 22, p. 1634-1645. Also, “hsa-mir-6756” (miRBase Accession No. MI0022601, SEQ ID NO: 299) having a hairpin-like structure is known as a precursor of “hsa-miR-6756-5p”.

The term “hsa-miR-373-5p gene” or “hsa-miR-373-5p” used herein includes the hsa-miR-373-5p gene (miRBase Accession No. MIMAT0000725) described in SEQ ID NO: 23, a homolog or an ortholog of a different organism species, and the like. The hsa-miR-373-5p gene can be obtained by a method described in Suh M R et al., 2004, Dev Biol, Vol. 270, p. 488-498. Also, “hsa-mir-373” (miRBase Accession No. M10000781, SEQ ID NO: 300) having a hairpin-like structure is known as a precursor of “hsa-miR-373-5p”.

The term “hsa-miR-1268a gene” or “hsa-miR-1268a” used herein includes the hsa-miR-1268a gene (miRBase Accession No. MIMAT0005922) described in SEQ ID NO: 24, a homolog or an ortholog of a different organism species, and the like. The hsa-miR-1268a gene can be obtained by a method described in Morin R D et al., 2008, Genome Res, Vol. 18, p. 610-621. Also, “hsa-mir-1268a” (miRBase Accession No. MI0006405, SEQ ID NO: 301) having a hairpin-like structure is known as a precursor of “hsa-miR-1268a”.

The term “hsa-miR-1260b gene” or “hsa-miR-1260b” used herein includes the hsa-miR-1260b gene (miRBase Accession No. MIMAT0015041) described in SEQ ID NO: 25, a homolog or an ortholog of a different organism species, and the like. The hsa-miR-1260b gene can be obtained by a method described in Stark M S et al., 2010, PLoS One, Vol. 5, e9685. Also, “hsa-mir-1260b” (miRBase Accession No. MI0014197, SEQ ID NO: 302) having a hairpin-like structure is known as a precursor of “hsa-miR-1260b”.

The term “hsa-miR-4258 gene” or “hsa-miR-4258” used herein includes the hsa-miR-4258 gene (miRBase Accession No. MIMAT0016879) described in SEQ ID NO: 26, a homolog or an ortholog of a different organism species, and the like. The hsa-miR-4258 gene can be obtained by a method described in Goff L A et al., 2009, PLoS One, Vol. 4, e7192. Also, “hsa-mir-4258” (miRBase Accession No. MI0015857, SEQ ID NO: 303) having a hairpin-like structure is known as a precursor of “hsa-miR-4258”.

The term “hsa-miR-4697-5p gene” or “hsa-miR-4697-5p” used herein includes the hsa-miR-4697-5p gene (miRBase Accession No. MIMAT0019791) described in SEQ ID NO: 27, a homolog or an ortholog of a different organism species, and the like. The hsa-miR-4697-5p gene can be obtained by a method described in Persson H et al., 2011, Cancer Res, Vol. 71, p. 78-86. Also, “hsa-mir-4697” (miRBase Accession No. MI0017330, SEQ ID NO: 304) having a hairpin-like structure is known as a precursor of “hsa-miR-4697-5p”.

The term “hsa-miR-1469 gene” or “hsa-miR-1469” used herein includes the hsa-miR-1469 gene (miRBase Accession No. MIMAT0007347) described in SEQ ID NO: 28, a homolog or an ortholog of a different organism species, and the like. The hsa-miR-1469 gene can be obtained by a method described in Kawaji H et al., 2008, BMC Genomics, Vol. 9, p. 157. Also, “hsa-mir-1469” (miRBase Accession No. M10007074, SEQ ID NO: 305) having a hairpin-like structure is known as a precursor of “hsa-miR-1469”.

The term “hsa-miR-4515 gene” or “hsa-miR-4515” used herein includes the hsa-miR-4515 gene (miRBase Accession No. MIMAT0019052) described in SEQ ID NO: 29, a homolog or an ortholog of a different organism species, and the like. The hsa-miR-4515 gene can be obtained by a method described in Jima D D et al., 2010, Blood, Vol. 116, e118-e127. Also, “hsa-mir-4515” (miRBase Accession No. MI0016881, SEQ ID NO: 306) having a hairpin-like structure is known as a precursor of “hsa-miR-4515”.

The term “hsa-miR-6861-5p gene” or “hsa-miR-6861-5p” used herein includes the hsa-miR-6861-5p gene (miRBase Accession No. MIMAT0027623) described in SEQ ID NO: 30, a homolog or an ortholog of a different organism species, and the like. The hsa-miR-6861-5p gene can be obtained by a method described in Ladewig E et al., 2012, Genome Res, Vol. 22, p. 1634-1645. Also, “hsa-mir-6861” (miRBase Accession No. MI0022708, SEQ ID NO: 307) having a hairpin-like structure is known as a precursor of “hsa-miR-6861-5p”.

The term “hsa-miR-6821-5p gene” or “hsa-miR-6821-5p” used herein includes the hsa-miR-6821-5p gene (miRBase Accession No. MIMAT0027542) described in SEQ ID NO: 31, a homolog or an ortholog of a different organism species, and the like. The hsa-miR-6821-5p gene can be obtained by a method described in Ladewig E et al., 2012, Genome Res, Vol. 22, p. 1634-1645. Also, “hsa-mir-6821” (miRBase Accession No. MI0022666, SEQ ID NO: 308) having a hairpin-like structure is known as a precursor of “hsa-miR-6821-5p”.

The term “hsa-miR-575 gene” or “hsa-miR-575” used herein includes the hsa-miR-575 gene (miRBase Accession No. MIMAT0003240) described in SEQ ID NO: 32, a homolog or an ortholog of a different organism species, and the like. The hsa-miR-575 gene can be obtained by a method described in Cummins J M et al., 2006, Proc Natl Ac ad Sci USA, Vol. 103, p. 3687-3692. Also, “hsa-mir-575” (miRBase Accession No. MI0003582, SEQ ID NO: 309) having a hairpin-like structure is known as a precursor of “hsa-miR-575”.

The term “hsa-miR-6805-5p gene” or “hsa-miR-6805-5p” used herein includes the hsa-miR-6805-5p gene (miRBase Accession No. MIMAT0027510) described in SEQ ID NO: 33, a homolog or an ortholog of a different organism species, and the like. The hsa-miR-6805-5p gene can be obtained by a method described in Ladewig E et al., 2012, Genome Res, Vol. 22, p. 1634-1645. Also, “hsa-mir-6805” (miRBase Accession No. MI0022650, SEQ ID NO: 310) having a hairpin-like structure is known as a precursor of “hsa-miR-6805-5p”.

The term “hsa-miR-4758-5p gene” or “hsa-miR-4758-5p” used herein includes the hsa-miR-4758-5p gene (miRBase Accession No. MIMAT0019903) described in SEQ ID NO: 34, a homolog or an ortholog of a different organism species, and the like. The hsa-miR-4758-5p gene can be obtained by a method described in Persson H et al., 2011, Cancer Res, Vol. 71, p. 78-86. Also, “hsa-mir-4758” (miRBase Accession No. MI0017399, SEQ ID NO: 311) having a hairpin-like structure is known as a precursor of “hsa-miR-4758-5p”.

The term “hsa-miR-3663-3p gene” or “hsa-miR-3663-3p” used herein includes the hsa-miR-3663-3p gene (miRBase Accession No. MIMAT0018085) described in SEQ ID NO: 35, a homolog or an ortholog of a different organism species, and the like. The hsa-miR-3663-3p gene can be obtained by a method described in Liao J Y et al., 2010, PLoS One, Vol. 5, e10563. Also, “hsa-mir-3663” (miRBase Accession No. MI0016064, SEQ ID NO: 312) having a hairpin-like structure is known as a precursor of “hsa-miR-3663-3p”.

The term “hsa-miR-4530 gene” or “hsa-miR-4530” used herein includes the hsa-miR-4530 gene (miRBase Accession No. MIMAT0019069) described in SEQ ID NO: 36, a homolog or an ortholog of a different organism species, and the like. The hsa-miR-4530 gene can be obtained by a method described in Jima D D et al., 2010, Blood, Vol. 116, e118-e127. Also, “hsa-mir-4530” (miRBase Accession No. MI0016897, SEQ ID NO: 313) having a hairpin-like structure is known as a precursor of “hsa-miR-4530”.

The term “hsa-miR-6798-5p gene” or “hsa-miR-6798-5p” used herein includes the hsa-miR-6798-5p gene (miRBase Accession No. MIMAT0027496) described in SEQ ID NO: 37, a homolog or an ortholog of a different organism species, and the like. The hsa-miR-6798-5p gene can be obtained by a method described in Ladewig E et al., 2012, Genome Res, Vol. 22, p. 1634-1645. Also, “hsa-mir-6798” (miRBase Accession No. MI0022643, SEQ ID NO: 314) having a hairpin-like structure is known as a precursor of “hsa-miR-6798-5p”.

The term “hsa-miR-6781-5p gene” or “hsa-miR-6781-5p” used herein includes the hsa-miR-6781-5p gene (miRBase Accession No. MIMAT0027462) described in SEQ ID NO: 38, a homolog or an ortholog of a different organism species, and the like. The hsa-miR-6781-5p gene can be obtained by a method described in Ladewig E et al., 2012, Genome Res, Vol. 22, p. 1634-1645. Also, “hsa-mir-6781” (miRBase Accession No. MI0022626, SEQ ID NO: 315) having a hairpin-like structure is known as a precursor of “hsa-miR-6781-5p”.

The term “hsa-miR-885-3p gene” or “hsa-miR-885-3p” used herein includes the hsa-miR-885-3p gene (miRBase Accession No. MIMAT0004948) described in SEQ ID NO: 39, a homolog or an ortholog of a different organism species, and the like. The hsa-miR-885-3p gene can be obtained by a method described in Berezikov E et al., 2006, Genome Res, Vol. 16, p. 1289-1298. Also, “hsa-mir-885” (miRBase Accession No. MI0005560, SEQ ID NO: 316) having a hairpin-like structure is known as a precursor of “hsa-miR-885-3p”.

The term “hsa-miR-1273g-3p gene” or “hsa-miR-1273g-3p” used herein includes the hsa-miR-1273g-3p gene (miRBase Accession No. MIMAT0022742) described in SEQ ID NO: 40, a homolog or an ortholog of a different organism species, and the like. The hsa-miR-1273g-3p gene can be obtained by a method described in Reshmi G et al., 2011, Genomics, Vol. 97, p. 333-340. Also, “hsa-mir-1273g” (miRBase Accession No. MI0018003, SEQ ID NO: 317) having a hairpin-like structure is known as a precursor of “hsa-miR-1273g-3p”.

The term “hsa-miR-4787-3p gene” or “hsa-miR-4787-3p” used herein includes the hsa-miR-4787-3p gene (miRBase Accession No. MIMAT0019957) described in SEQ ID NO: 41, a homolog or an ortholog of a different organism species, and the like. The hsa-miR-4787-3p gene can be obtained by a method described in Persson H et al., 2011, Cancer Res, Vol. 71, p. 78-86. Also, “hsa-mir-4787” (miRBase Accession No. MI0017434, SEQ ID NO: 318) having a hairpin-like structure is known as a precursor of “hsa-miR-4787-3p”.

The term “hsa-miR-4454 gene” or “hsa-miR-4454” used herein includes the hsa-miR-4454 gene (miRBase Accession No. MIMAT0018976) described in SEQ ID NO: 42, a homolog or an ortholog of a different organism species, and the like. The hsa-miR-4454 gene can be obtained by a method described in Jima D D et al., 2010, Blood, Vol. 116, e118-e127. Also, “hsa-mir-4454” (miRBase Accession No. MI0016800, SEQ ID NO: 319) having a hairpin-like structure is known as a precursor of “hsa-miR-4454”.

The term “hsa-miR-4706 gene” or “hsa-miR-4706” used herein includes the hsa-miR-4706 gene (miRBase Accession No. MIMAT0019806) described in SEQ ID NO: 43, a homolog or an ortholog of a different organism species, and the like. The hsa-miR-4706 gene can be obtained by a method described in Persson H et al., 2011, Cancer Res, Vol. 71, p. 78-86. Also, “hsa-mir-4706” (miRBase Accession No. M10017339, SEQ ID NO: 320) having a hairpin-like structure is known as a precursor of “hsa-miR-4706”.

The term “hsa-miR-1249-3p gene” or “hsa-miR-1249-3p” used herein includes the hsa-miR-1249-3p gene (miRBase Accession No. MIMAT0005901) described in SEQ ID NO: 44, a homolog or an ortholog of a different organism species, and the like. The hsa-miR-1249-3p gene can be obtained by a method described in Morin R D et al., 2008, Genome Res, Vol. 18, p. 610-621. Also, “hsa-mir-1249” (miRBase Accession No. MI0006384, SEQ ID NO: 321) having a hairpin-like structure is known as a precursor of “hsa-miR-1249-3p”.

The term “hsa-miR-887-3p gene” or “hsa-miR-887-3p” used herein includes the hsa-miR-887-3p gene (miRBase Accession No. MIMAT0004951) described in SEQ ID NO: 45, a homolog or an ortholog of a different organism species, and the like. The hsa-miR-887-3p gene can be obtained by a method described in Berezikov E et al., 2006, Genome Res, Vol. 16, p. 1289-1298. Also, “hsa-mir-887” (miRBase Accession No. MI0005562, SEQ ID NO: 322) having a hairpin-like structure is known as a precursor of “hsa-miR-887-3p”.

The term “hsa-miR-6786-5p gene” or “hsa-miR-6786-5p” used herein includes the hsa-miR-6786-5p gene (miRBase Accession No. MIMAT0027472) described in SEQ ID NO: 46, a homolog or an ortholog of a different organism species, and the like. The hsa-miR-6786-5p gene can be obtained by a method described in Ladewig E et al., 2012, Genome Res, Vol. 22, p. 1634-1645. Also, “hsa-mir-6786” (miRBase Accession No. MI0022631, SEQ ID NO: 323) having a hairpin-like structure is known as a precursor of “hsa-miR-6786-5p”.

The term “hsa-miR-1238-5p gene” or “hsa-miR-1238-5p” used herein includes the hsa-miR-1238-5p gene (miRBase Accession No. MIMAT0022947) described in SEQ ID NO: 47, a homolog or an ortholog of a different organism species, and the like. The hsa-miR-1238-5p gene can be obtained by a method described in Berezikov E et al., 2007, Mol Cell, Vol. 28, p. 328-336. Also, “hsa-mir-1238” (miRBase Accession No. MI0006328, SEQ ID NO: 324) having a hairpin-like structure is known as a precursor of “hsa-miR-1238-5p”.

The term “hsa-miR-6749-5p gene” or “hsa-miR-6749-5p” used herein includes the hsa-miR-6749-5p gene (miRBase Accession No. MIMAT0027398) described in SEQ ID NO: 48, a homolog or an ortholog of a different organism species, and the like. The hsa-miR-6749-5p gene can be obtained by a method described in Ladewig E et al., 2012, Genome Res, Vol. 22, p. 1634-1645. Also, “hsa-mir-6749” (miRBase Accession No. MI0022594, SEQ ID NO: 325) having a hairpin-like structure is known as a precursor of “hsa-miR-6749-5p”.

The term “hsa-miR-6729-5p gene” or “hsa-miR-6729-5p” used herein includes the hsa-miR-6729-5p gene (miRBase Accession No. MIMAT0027359) described in SEQ ID NO: 49, a homolog or an ortholog of a different organism species, and the like. The hsa-miR-6729-5p gene can be obtained by a method described in Ladewig E et al., 2012, Genome Res, Vol. 22, p. 1634-1645. Also, “hsa-mir-6729” (miRBase Accession No. MI0022574, SEQ ID NO: 326) having a hairpin-like structure is known as a precursor of “hsa-miR-6729-5p”.

The term “hsa-miR-6825-5p gene” or “hsa-miR-6825-5p” used herein includes the hsa-miR-6825-5p gene (miRBase Accession No. MIMAT0027550) described in SEQ ID NO: 50, a homolog or an ortholog of a different organism species, and the like. The hsa-miR-6825-5p gene can be obtained by a method described in Ladewig E et al., 2012, Genome Res, Vol. 22, p. 1634-1645. Also, “hsa-mir-6825” (miRBase Accession No. MI0022670, SEQ ID NO: 327) having a hairpin-like structure is known as a precursor of “hsa-miR-6825-5p”.

The term “hsa-miR-663b gene” or “hsa-miR-663b” used herein includes the hsa-miR-663b gene (miRBase Accession No. MIMAT0005867) described in SEQ ID NO: 51, a homolog or an ortholog of a different organism species, and the like. The hsa-miR-663b gene can be obtained by a method described in Takada S et al., 2008, Leukemia, Vol. 22, p. 1274-1278. Also, “hsa-mir-663b” (miRBase Accession No. MI0006336, SEQ ID NO: 328) having a hairpin-like structure is known as a precursor of “hsa-miR-663b”.

The term “hsa-miR-6858-5p gene” or “hsa-miR-6858-5p” used herein includes the hsa-miR-6858-5p gene (miRBase Accession No. MIMAT0027616) described in SEQ ID NO: 52, a homolog or an ortholog of a different organism species, and the like. The hsa-miR-6858-5p gene can be obtained by a method described in Ladewig E et al., 2012, Genome Res, Vol. 22, p. 1634-1645. Also, “hsa-mir-6858” (miRBase Accession No. MI0022704, SEQ ID NO: 329) having a hairpin-like structure is known as a precursor of “hsa-miR-6858-5p”.

The term “hsa-miR-4690-5p gene” or “hsa-miR-4690-5p” used herein includes the hsa-miR-4690-5p gene (miRBase Accession No. MIMAT0019779) described in SEQ ID NO: 53, a homolog or an ortholog of a different organism species, and the like. The hsa-miR-4690-5p gene can be obtained by a method described in Persson H et al., 2011, Cancer Res, Vol. 71, p. 78-86. Also, “hsa-mir-4690” (miRBase Accession No. MI0017323, SEQ ID NO: 330) having a hairpin-like structure is known as a precursor of “hsa-miR-4690-5p”.

The term “hsa-miR-6765-5p gene” or “hsa-miR-6765-5p” used herein includes the hsa-miR-6765-5p gene (miRBase Accession No. MIMAT0027430) described in SEQ ID NO: 54, a homolog or an ortholog of a different organism species, and the like. The hsa-miR-6765-5p gene can be obtained by a method described in Ladewig E et al., 2012, Genome Res, Vol. 22, p. 1634-1645. Also, “hsa-mir-6765” (miRBase Accession No. MI0022610, SEQ ID NO: 331) having a hairpin-like structure is known as a precursor of “hsa-miR-6765-5p”.

The term “hsa-miR-4710 gene” or “hsa-miR-4710” used herein includes the hsa-miR-4710 gene (miRBase Accession No. MIMAT0019815) described in SEQ ID NO: 55, a homolog or an ortholog of a different organism species, and the like. The hsa-miR-4710 gene can be obtained by a method described in Persson H et al., 2011, Cancer Res, Vol. 71, p. 78-86. Also, “hsa-mir-4710” (miRBase Accession No. MI0017344, SEQ ID NO: 332) having a hairpin-like structure is known as a precursor of “hsa-miR-4710”.

The term “hsa-miR-6775-5p gene” or “hsa-miR-6775-5p” used herein includes the hsa-miR-6775-5p gene (miRBase Accession No. MIMAT0027450) described in SEQ ID NO: 56, a homolog or an ortholog of a different organism species, and the like. The hsa-miR-6775-5p gene can be obtained by a method described in Ladewig E et al., 2012, Genome Res, Vol. 22, p. 1634-1645. Also, “hsa-mir-6775” (miRBase Accession No. MI0022620, SEQ ID NO: 333) having a hairpin-like structure is known as a precursor of “hsa-miR-6775-5p”.

The term “hsa-miR-371a-5p gene” or “hsa-miR-371a-5p” used herein includes the hsa-miR-371a-5p gene (miRBase Accession No. MIMAT0004687) described in SEQ ID NO: 57, a homolog or an ortholog of a different organism species, and the like. The hsa-miR-371a-5p gene can be obtained by a method described in Suh M R et al., 2004, Dev Biol, Vol. 270, p. 488-498. Also, “hsa-mir-371a” (miRBase Accession No. MI0000779, SEQ ID NO: 334) having a hairpin-like structure is known as a precursor of “hsa-miR-371a-5p”.

The term “hsa-miR-6816-5p gene” or “hsa-miR-6816-5p” used herein includes the hsa-miR-6816-5p gene (miRBase Accession No. MIMAT0027532) described in SEQ ID NO: 58, a homolog or an ortholog of a different organism species, and the like. The hsa-miR-6816-5p gene can be obtained by a method described in Ladewig E et al., 2012, Genome Res, Vol. 22, p. 1634-1645. Also, “hsa-mir-6816” (miRBase Accession No. MI0022661, SEQ ID NO: 335) having a hairpin-like structure is known as a precursor of “hsa-miR-6816-5p”.

The term “hsa-miR-296-3p gene” or “hsa-miR-296-3p” used herein includes the hsa-miR-296-3p gene (miRBase Accession No. MIMAT0004679) described in SEQ ID NO: 59, a homolog or an ortholog of a different organism species, and the like. The hsa-miR-296-3p gene can be obtained by a method described in Houbaviy H B et al., 2003, Dev Cell, Vol. 5, p. 351-358. Also, “hsa-mir-296” (miRBase Accession No. MI0000747, SEQ ID NO: 336) having a hairpin-like structure is known as a precursor of “hsa-miR-296-3p”.

The term “hsa-miR-7977 gene” or “hsa-miR-7977” used herein includes the hsa-miR-7977 gene (miRBase Accession No. MIMAT0031180) described in SEQ ID NO: 60, a homolog or an ortholog of a different organism species, and the like. The hsa-miR-7977 gene can be obtained by a method described in Velthut-Meikas A et al., 2013, Mol Endocrinol, Vol. 27, p. 1128-1141. Also, “hsa-mir-7977” (miRBase Accession No. MI0025753, SEQ ID NO: 337) having a hairpin-like structure is known as a precursor of “hsa-miR-7977”.

The term “hsa-miR-8069 gene” or “hsa-miR-8069” used herein includes the hsa-miR-8069 gene (miRBase Accession No. MIMAT0030996) described in SEQ ID NO: 61, a homolog or an ortholog of a different organism species, and the like. The hsa-miR-8069 gene can be obtained by a method described in Wang H J et al., 2013, Shock, Vol. 39, p. 480-487. Also, “hsa-mir-8069-1 and hsa-mir-8069-2” (miRBase Accession Nos. MI0025905 and MI0031519, SEQ ID NOs: 338 and 339) having a hairpin-like structure are known as precursors of “hsa-miR-8069”.

The term “hsa-miR-6515-3p gene” or “hsa-miR-6515-3p” used herein includes the hsa-miR-6515-3p gene (miRBase Accession No. MIMAT0025487) described in SEQ ID NO: 62, a homolog or an ortholog of a different organism species, and the like. The hsa-miR-6515-3p gene can be obtained by a method described in Joyce C E et al., 2011, Hum Mol Genet, Vol. 20, p. 4025-4040. Also, “hsa-mir-6515” (miRBase Accession No. MI0022227, SEQ ID NO: 340) having a hairpin-like structure is known as a precursor of “hsa-miR-6515-3p”.

The term “hsa-miR-4687-5p gene” or “hsa-miR-4687-5p” used herein includes the hsa-miR-4687-5p gene (miRBase Accession No. MIMAT0019774) described in SEQ ID NO: 63, a homolog or an ortholog of a different organism species, and the like. The hsa-miR-4687-5p gene can be obtained by a method described in Persson H et al., 2011, Cancer Res, Vol. 71, p. 78-86. Also, “hsa-mir-4687” (miRBase Accession No. MI0017319, SEQ ID NO: 341) having a hairpin-like structure is known as a precursor of “hsa-miR-4687-5p”.

The term “hsa-miR-1343-5p gene” or “hsa-miR-1343-5p” used herein includes the hsa-miR-1343-5p gene (miRBase Accession No. MIMAT0027038) described in SEQ ID NO: 64, a homolog or an ortholog of a different organism species, and the like. The hsa-miR-1343-5p gene can be obtained by a method described in Persson H et al., 2011, Cancer Res, Vol. 71, p. 78-86. Also, “hsa-mir-1343” (miRBase Accession No. MI0017320, SEQ ID NO: 294) having a hairpin-like structure is known as a precursor of “hsa-miR-1343-5p”.

The term “hsa-miR-7110-5p gene” or “hsa-miR-7110-5p” used herein includes the hsa-miR-7110-5p gene (miRBase Accession No. MIMAT0028117) described in SEQ ID NO: 65, a homolog or an ortholog of a different organism species, and the like. The hsa-miR-7110-5p gene can be obtained by a method described in Ladewig E et al., 2012, Genome Res, Vol. 22, p. 1634-1645. Also, “hsa-mir-7110” (miRBase Accession No. MI0022961, SEQ ID NO: 342) having a hairpin-like structure is known as a precursor of “hsa-miR-7110-5p”.

The term “hsa-miR-4525 gene” or “hsa-miR-4525” used herein includes the hsa-miR-4525 gene (miRBase Accession No. MIMAT0019064) described in SEQ ID NO: 66, a homolog or an ortholog of a different organism species, and the like. The hsa-miR-4525 gene can be obtained by a method described in Jima D D et al., 2010, Blood, Vol. 116, e118-e127. Also, “hsa-mir-4525” (miRBase Accession No. MI0016892, SEQ ID NO: 343) having a hairpin-like structure is known as a precursor of “hsa-miR-4525”.

The term “hsa-miR-3158-5p gene” or “hsa-miR-3158-5p” used herein includes the hsa-miR-3158-5p gene (miRBase Accession No. MIMAT0019211) described in SEQ ID NO: 67, a homolog or an ortholog of a different organism species, and the like. The hsa-miR-3158-5p gene can be obtained by a method described in Creighton C J et al., 2010, PLoS One, Vol. 5, e9637. Also, “hsa-mir-3158-1 and hsa-mir-3158-2” (miRBase Accession Nos. MI0014186 and MI0014187, SEQ ID NOs: 344 and 345) having a hairpin-like structure are known as precursors of “hsa-miR-3158-5p”.

The term “hsa-miR-6787-5p gene” or “hsa-miR-6787-5p” used herein includes the hsa-miR-6787-5p gene (miRBase Accession No. MIMAT0027474) described in SEQ ID NO: 68, a homolog or an ortholog of a different organism species, and the like. The hsa-miR-6787-5p gene can be obtained by a method described in Ladewig E et al., 2012, Genome Res, Vol. 22, p. 1634-1645. Also, “hsa-mir-6787” (miRBase Accession No. MI0022632, SEQ ID NO: 346) having a hairpin-like structure is known as a precursor of “hsa-miR-6787-5p”.

The term “hsa-miR-614 gene” or “hsa-miR-614” used herein includes the hsa-miR-614 gene (miRBase Accession No. MIMAT0003282) described in SEQ ID NO: 69, a homolog or an ortholog of a different organism species, and the like. The hsa-miR-614 gene can be obtained by a method described in Cummins J M et al., 2006, Proc Natl Acad Sci USA, Vol. 103, p. 3687-3692. Also, “hsa-mir-614” (miRBase Accession No. MI0003627, SEQ ID NO: 347) having a hairpin-like structure is known as a precursor of “hsa-miR-614”.

The term “hsa-miR-4689 gene” or “hsa-miR-4689” used herein includes the hsa-miR-4689 gene (miRBase Accession No. MIMAT0019778) described in SEQ ID NO: 70, a homolog or an ortholog of a different organism species, and the like. The hsa-miR-4689 gene can be obtained by a method described in Persson H et al., 2011, Cancer Res, Vol. 71, p. 78-86. Also, “hsa-mir-4689” (miRBase Accession No. M10017322, SEQ ID NO: 348) having a hairpin-like structure is known as a precursor of “hsa-miR-4689”.

The term “hsa-miR-1185-2-3p gene” or “hsa-miR-1185-2-3p” used herein includes the hsa-miR-1185-2-3p gene (miRBase Accession No. MIMAT0022713) described in SEQ ID NO: 71, a homolog or an ortholog of a different organism species, and the like. The hsa-miR-1185-2-3p gene can be obtained by a method described in Berezikov E et al., 2006, Genome Res, Vol. 16, p. 1289-1298. Also, “hsa-mir-1185-2” (miRBase Accession No. MI0003821, SEQ ID NO: 349) having a hairpin-like structure is known as a precursor of “hsa-miR-1185-2-3p”.

The term “hsa-miR-1268b gene” or “hsa-miR-1268b” used herein includes the hsa-miR-1268b gene (miRBase Accession No. MIMAT0018925) described in SEQ ID NO: 72, a homolog or an ortholog of a different organism species, and the like. The hsa-miR-1268b gene can be obtained by a method described in Jima D D et al., 2010, Blood, Vol. 116, e118-e127. Also, “hsa-mir-1268b” (miRBase Accession No. MI0016748, SEQ ID NO: 350) having a hairpin-like structure is known as a precursor of “hsa-miR-1268b”.

The term “hsa-miR-1228-3p gene” or “hsa-miR-1228-3p” used herein includes the hsa-miR-1228-3p gene (miRBase Accession No. MIMAT0005583) described in SEQ ID NO: 73, a homolog or an ortholog of a different organism species, and the like. The hsa-miR-1228-3p gene can be obtained by a method described in Berezikov E et al., 2007, Mol Cell, Vol. 28, p. 328-336. Also, “hsa-mir-1228” (miRBase Accession No. MI0006318, SEQ ID NO: 278) having a hairpin-like structure is known as a precursor of “hsa-miR-1228-3p”.

The term “hsa-miR-1185-1-3p gene” or “hsa-miR-1185-1-3p” used herein includes the hsa-miR-1185-1-3p gene (miRBase Accession No. MIMAT0022838) described in SEQ ID NO: 74, a homolog or an ortholog of a different organism species, and the like. The hsa-miR-1185-1-3p gene can be obtained by a method described in Berezikov E et al., 2006, Genome Res, Vol. 16, p. 1289-1298. Also, “hsa-mir-1185-1” (miRBase Accession No. MI0003844, SEQ ID NO: 351) having a hairpin-like structure is known as a precursor of “hsa-miR-1185-1-3p”.

The term “hsa-miR-940 gene” or “hsa-miR-940” used herein includes the hsa-miR-940 gene (miRBase Accession No. MIMAT0004983) described in SEQ ID NO: 75, a homolog or an ortholog of a different organism species, and the like. The hsa-miR-940 gene can be obtained by a method described in Lui W O et al., 2007, Cancer Res, Vol. 67, p. 6031-6043. Also, “hsa-mir-940” (miRBase Accession No. MI0005762, SEQ ID NO: 352) having a hairpin-like structure is known as a precursor of “hsa-miR-940”.

The term “hsa-miR-939-5p gene” or “hsa-miR-939-5p” used herein includes the hsa-miR-939-5p gene (miRBase Accession No. MIMAT0004982) described in SEQ ID NO: 76, a homolog or an ortholog of a different organism species, and the like. The hsa-miR-939-5p gene can be obtained by a method described in Lui W O et al., 2007, Cancer Res, Vol. 67, p. 6031-6043. Also, “hsa-mir-939” (miRBase Accession No. MI0005761, SEQ ID NO: 353) having a hairpin-like structure is known as a precursor of “hsa-miR-939-5p”.

The term “hsa-miR-6757-5p gene” or “hsa-miR-6757-5p” used herein includes the hsa-miR-6757-5p gene (miRBase Accession No. MIMAT0027414) described in SEQ ID NO: 77, a homolog or an ortholog of a different organism species, and the like. The hsa-miR-6757-5p gene can be obtained by a method described in Ladewig E et al., 2012, Genome Res, Vol. 22, p. 1634-1645. Also, “hsa-mir-6757” (miRBase Accession No. MI0022602, SEQ ID NO: 354) having a hairpin-like structure is known as a precursor of “hsa-miR-6757-5p”.

The term “hsa-miR-1275 gene” or “hsa-miR-1275” used herein includes the hsa-miR-1275 gene (miRBase Accession No. MIMAT0005929) described in SEQ ID NO: 78, a homolog or an ortholog of a different organism species, and the like. The hsa-miR-1275 gene can be obtained by a method described in Morin R D et al., 2008, Genome Res, Vol. 18, p. 610-621. Also, “hsa-mir-1275” (miRBase Accession No. M10006415, SEQ ID NO: 355) having a hairpin-like structure is known as a precursor of “hsa-miR-1275”.

The term “hsa-miR-5001-5p gene” or “hsa-miR-5001-5p” used herein includes the hsa-miR-5001-5p gene (miRBase Accession No. MIMAT0021021) described in SEQ ID NO: 79, a homolog or an ortholog of a different organism species, and the like. The hsa-miR-5001-5p gene can be obtained by a method described in Hansen T B et al., 2011, RNA Biol, Vol. 8, p. 378-383. Also, “hsa-mir-5001” (miRBase Accession No. MI0017867, SEQ ID NO: 356) having a hairpin-like structure is known as a precursor of “hsa-miR-5001-5p”.

The term “hsa-miR-6826-5p gene” or “hsa-miR-6826-5p” used herein includes the hsa-miR-6826-5p gene (miRBase Accession No. MIMAT0027552) described in SEQ ID NO: 80, a homolog or an ortholog of a different organism species, and the like. The hsa-miR-6826-5p gene can be obtained by a method described in Ladewig E et al., 2012, Genome Res, Vol. 22, p. 1634-1645. Also, “hsa-mir-6826” (miRBase Accession No. MI0022671, SEQ ID NO: 357) having a hairpin-like structure is known as a precursor of “hsa-miR-6826-5p”.

The term “hsa-miR-6765-3p gene” or “hsa-miR-6765-3p” used herein includes the hsa-miR-6765-3p gene (miRBase Accession No. MIMAT0027431) described in SEQ ID NO: 81, a homolog or an ortholog of a different organism species, and the like. The hsa-miR-6765-3p gene can be obtained by a method described in Ladewig E et al., 2012, Genome Res, Vol. 22, p. 1634-1645. Also, “hsa-mir-6765” (miRBase Accession No. MI0022610, SEQ ID NO: 331) having a hairpin-like structure is known as a precursor of “hsa-miR-6765-3p”.

The term “hsa-miR-3679-3p gene” or “hsa-miR-3679-3p” used herein includes the hsa-miR-3679-3p gene (miRBase Accession No. MIMAT0018105) described in SEQ ID NO: 82, a homolog or an ortholog of a different organism species, and the like. The hsa-miR-3679-3p gene can be obtained by a method described in Creighton C J et al., 2010, PLoS One, Vol. 5, e9637. Also, “hsa-mir-3679” (miRBase Accession No. MI0016080, SEQ ID NO: 358) having a hairpin-like structure is known as a precursor of “hsa-miR-3679-3p”.

The term “hsa-miR-4718 gene” or “hsa-miR-4718” used herein includes the hsa-miR-4718 gene (miRBase Accession No. MIMAT0019831) described in SEQ ID NO: 83, a homolog or an ortholog of a different organism species, and the like. The hsa-miR-4718 gene can be obtained by a method described in Persson H et al., 2011, Cancer Res, Vol. 71, p. 78-86. Also, “hsa-mir-4718” (miRBase Accession No. M10017353, SEQ ID NO: 359) having a hairpin-like structure is known as a precursor of “hsa-miR-4718”.

The term “hsa-miR-4286 gene” or “hsa-miR-4286” used herein includes the hsa-miR-4286 gene (miRBase Accession No. MIMAT0016916) described in SEQ ID NO: 84, a homolog or an ortholog of a different organism species, and the like. The hsa-miR-4286 gene can be obtained by a method described in Goff L A et al., 2009, PLoS One, Vol. 4, e7192. Also, “hsa-mir-4286” (miRBase Accession No. MI0015894, SEQ ID NO: 360) having a hairpin-like structure is known as a precursor of “hsa-miR-4286”.

The term “hsa-miR-8059 gene” or “hsa-miR-8059” used herein includes the hsa-miR-8059 gene (miRBase Accession No. MIMAT0030986) described in SEQ ID NO: 85, a homolog or an ortholog of a different organism species, and the like. The hsa-miR-8059 gene can be obtained by a method described in Wang H J et al., 2013, Shock, Vol. 39, p. 480-487. Also, “hsa-mir-8059” (miRBase Accession No. MI0025895, SEQ ID NO: 361) having a hairpin-like structure is known as a precursor of “hsa-miR-8059”.

The term “hsa-miR-4447 gene” or “hsa-miR-4447” used herein includes the hsa-miR-4447 gene (miRBase Accession No. MIMAT0018966) described in SEQ ID NO: 86, a homolog or an ortholog of a different organism species, and the like. The hsa-miR-4447 gene can be obtained by a method described in Jima D D et al., 2010, Blood, Vol. 116, e118-e127. Also, “hsa-mir-4447” (miRBase Accession No. MI0016790, SEQ ID NO: 362) having a hairpin-like structure is known as a precursor of “hsa-miR-4447”.

The term “hsa-miR-4448 gene” or “hsa-miR-4448” used herein includes the hsa-miR-4448 gene (miRBase Accession No. MIMAT0018967) described in SEQ ID NO: 87, a homolog or an ortholog of a different organism species, and the like. The hsa-miR-4448 gene can be obtained by a method described in Jima D D et al., 2010, Blood, Vol. 116, e118-e127. Also, “hsa-mir-4448” (miRBase Accession No. MI0016791, SEQ ID NO: 363) having a hairpin-like structure is known as a precursor of “hsa-miR-4448”.

The term “hsa-miR-658 gene” or “hsa-miR-658” used herein includes the hsa-miR-658 gene (miRBase Accession No. MIMAT0003336) described in SEQ ID NO: 88, a homolog or an ortholog of a different organism species, and the like. The hsa-miR-658 gene can be obtained by a method described in Cummins J M et al., 2006, Proc Natl Acad Sci USA, Vol. 103, p. 3687-3692. Also, “hsa-mir-658” (miRBase Accession No. MI0003682, SEQ ID NO: 364) having a hairpin-like structure is known as a precursor of “hsa-miR-658”.

The term “hsa-miR-6766-3p gene” or “hsa-miR-6766-3p” used herein includes the hsa-miR-6766-3p gene (miRBase Accession No. MIMAT0027433) described in SEQ ID NO: 89, a homolog or an ortholog of a different organism species, and the like. The hsa-miR-6766-3p gene can be obtained by a method described in Ladewig E et al., 2012, Genome Res, Vol. 22, p. 1634-1645. Also, “hsa-mir-6766” (miRBase Accession No. MI0022611, SEQ ID NO: 365) having a hairpin-like structure is known as a precursor of “hsa-miR-6766-3p”.

The term “hsa-miR-197-5p gene” or “hsa-miR-197-5p” used herein includes the hsa-miR-197-5p gene (miRBase Accession No. MIMAT0022691) described in SEQ ID NO: 90, a homolog or an ortholog of a different organism species, and the like. The hsa-miR-197-5p gene can be obtained by a method described in Lagos-Quintana M et al., 2003, RNA, Vol. 9, p. 175-179. Also, “hsa-mir-197” (miRBase Accession No. MI0000239, SEQ ID NO: 366) having a hairpin-like structure is known as a precursor of “hsa-miR-197-5p”.

The term “hsa-miR-6887-5p gene” or “hsa-miR-6887-5p” used herein includes the hsa-miR-6887-5p gene (miRBase Accession No. MIMAT0027674) described in SEQ ID NO: 91, a homolog or an ortholog of a different organism species, and the like. The hsa-miR-6887-5p gene can be obtained by a method described in Ladewig E et al., 2012, Genome Res, Vol. 22, p. 1634-1645. Also, “hsa-mir-6887” (miRBase Accession No. MI0022734, SEQ ID NO: 367) having a hairpin-like structure is known as a precursor of “hsa-miR-6887-5p”.

The term “hsa-miR-6742-5p gene” or “hsa-miR-6742-5p” used herein includes the hsa-miR-6742-5p gene (miRBase Accession No. MIMAT0027385) described in SEQ ID NO: 92, a homolog or an ortholog of a different organism species, and the like. The hsa-miR-6742-5p gene can be obtained by a method described in Ladewig E et al., 2012, Genome Res, Vol. 22, p. 1634-1645. Also, “hsa-mir-6742” (miRBase Accession No. MI0022587, SEQ ID NO: 368) having a hairpin-like structure is known as a precursor of “hsa-miR-6742-5p”.

The term “hsa-miR-6729-3p gene” or “hsa-miR-6729-3p” used herein includes the hsa-miR-6729-3p gene (miRBase Accession No. MIMAT0027360) described in SEQ ID NO: 93, a homolog or an ortholog of a different organism species, and the like. The hsa-miR-6729-3p gene can be obtained by a method described in Ladewig E et al., 2012, Genome Res, Vol. 22, p. 1634-1645. Also, “hsa-mir-6729” (miRBase Accession No. MI0022574, SEQ ID NO: 326) having a hairpin-like structure is known as a precursor of “hsa-miR-6729-3p”.

The term “hsa-miR-5090 gene” or “hsa-miR-5090” used herein includes the hsa-miR-5090 gene (miRBase Accession No. MIMAT0021082) described in SEQ ID NO: 94, a homolog or an ortholog of a different organism species, and the like. The hsa-miR-5090 gene can be obtained by a method described in Ding N et al., 2011, J Radiat Res, Vol. 52, p. 425-432. Also, “hsa-mir-5090” (miRBase Accession No. MI0017979, SEQ ID NO: 369) having a hairpin-like structure is known as a precursor of “hsa-miR-5090”.

The term “hsa-miR-7975 gene” or “hsa-miR-7975” used herein includes the hsa-miR-7975 gene (miRBase Accession No. MIMAT0031178) described in SEQ ID NO: 95, a homolog or an ortholog of a different organism species, and the like. The hsa-miR-7975 gene can be obtained by a method described in Velthut-Meikas A et al., 2013, Mol Endocrinol, Vol. 27, p. 1128-1141. Also, “hsa-mir-7975” (miRBase Accession No. MI0025751, SEQ ID NO: 370) having a hairpin-like structure is known as a precursor of “hsa-miR-7975”.

The term “hsa-miR-4505 gene” or “hsa-miR-4505” used herein includes the hsa-miR-4505 gene (miRBase Accession No. MIMAT0019041) described in SEQ ID NO: 96, a homolog or an ortholog of a different organism species, and the like. The hsa-miR-4505 gene can be obtained by a method described in Jima D D et al., 2010, Blood, Vol. 116, e118-e127. Also, “hsa-mir-4505” (miRBase Accession No. MI0016868, SEQ ID NO: 371) having a hairpin-like structure is known as a precursor of “hsa-miR-4505”.

The term “hsa-miR-6889-5p gene” or “hsa-miR-6889-5p” used herein includes the hsa-miR-6889-5p gene (miRBase Accession No. MIMAT0027678) described in SEQ ID NO: 97, a homolog or an ortholog of a different organism species, and the like. The hsa-miR-6889-5p gene can be obtained by a method described in Ladewig E et al., 2012, Genome Res, Vol. 22, p. 1634-1645. Also, “hsa-mir-6889” (miRBase Accession No. MI0022736, SEQ ID NO: 372) having a hairpin-like structure is known as a precursor of “hsa-miR-6889-5p”.

The term “hsa-miR-4708-3p gene” or “hsa-miR-4708-3p” used herein includes the hsa-miR-4708-3p gene (miRBase Accession No. MIMAT0019810) described in SEQ ID NO: 98, a homolog or an ortholog of a different organism species, and the like. The hsa-miR-4708-3p gene can be obtained by a method described in Persson H et al., 2011, Cancer Res, Vol. 71, p. 78-86. Also, “hsa-mir-4708” (miRBase Accession No. MI0017341, SEQ ID NO: 373) having a hairpin-like structure is known as a precursor of “hsa-miR-4708-3p”.

The term “hsa-miR-6131 gene” or “hsa-miR-6131” used herein includes the hsa-miR-6131 gene (miRBase Accession No. MIMAT0024615) described in SEQ ID NO: 99, a homolog or an ortholog of a different organism species, and the like. The hsa-miR-6131 gene can be obtained by a method described in Dannemann M et al., 2012, Genome Biol Evol, Vol. 4, p. 552-564. Also, “hsa-mir-6131” (miRBase Accession No. MI0021276, SEQ ID NO: 374) having a hairpin-like structure is known as a precursor of “hsa-miR-6131”.

The term “hsa-miR-1225-3p gene” or “hsa-miR-1225-3p” used herein includes the hsa-miR-1225-3p gene (miRBase Accession No. MIMAT0005573) described in SEQ ID NO: 100, a homolog or an ortholog of a different organism species, and the like. The hsa-miR-1225-3p gene can be obtained by a method described in Berezikov E et al., 2007, Mol Cell, Vol. 28, p. 328-336. Also, “hsa-mir-1225” (miRBase Accession No. MI0006311, SEQ ID NO: 375) having a hairpin-like structure is known as a precursor of “hsa-miR-1225-3p”.

The term “hsa-miR-6132 gene” or “hsa-miR-6132” used herein includes the hsa-miR-6132 gene (miRBase Accession No. MIMAT0024616) described in SEQ ID NO: 101, a homolog or an ortholog of a different organism species, and the like. The hsa-miR-6132 gene can be obtained by a method described in Dannemann M et al., 2012, Genome Biol Evol, Vol. 4, p. 552-564. Also, “hsa-mir-6132” (miRBase Accession No. MI0021277, SEQ ID NO: 376) having a hairpin-like structure is known as a precursor of “hsa-miR-6132”.

The term “hsa-miR-4734 gene” or “hsa-miR-4734” used herein includes the hsa-miR-4734 gene (miRBase Accession No. MIMAT0019859) described in SEQ ID NO: 102, a homolog or an ortholog of a different organism species, and the like. The hsa-miR-4734 gene can be obtained by a method described in Persson H et al., 2011, Cancer Res, Vol. 71, p. 78-86. Also, “hsa-mir-4734” (miRBase Accession No. MI0017371, SEQ ID NO: 377) having a hairpin-like structure is known as a precursor of “hsa-miR-4734”.

The term “hsa-miR-3194-3p gene” or “hsa-miR-3194-3p” used herein includes the hsa-miR-3194-3p gene (miRBase Accession No. MIMAT0019218) described in SEQ ID NO: 103, a homolog or an ortholog of a different organism species, and the like. The hsa-miR-3194-3p gene can be obtained by a method described in Stark M S et al., 2010, PLoS One, Vol. 5, e9685. Also, “hsa-mir-3194” (miRBase Accession No. MI0014239, SEQ ID NO: 378) having a hairpin-like structure is known as a precursor of “hsa-miR-3194-3p”.

The term “hsa-miR-638 gene” or “hsa-miR-638” used herein includes the hsa-miR-638 gene (miRBase Accession No. MIMAT0003308) described in SEQ ID NO: 104, a homolog or an ortholog of a different organism species, and the like. The hsa-miR-638 gene can be obtained by a method described in Cummins J M et al., 2006, Proc Natl Acad Sci USA, Vol. 103, p. 3687-3692. Also, “hsa-mir-638” (miRBase Accession No. MI0003653, SEQ ID NO: 379) having a hairpin-like structure is known as a precursor of “hsa-miR-638”.

The term “hsa-miR-2467-3p gene” or “hsa-miR-2467-3p” used herein includes the hsa-miR-2467-3p gene (miRBase Accession No. MIMAT0019953) described in SEQ ID NO: 105, a homolog or an ortholog of a different organism species, and the like. The hsa-miR-2467-3p gene can be obtained by a method described in Persson H et al., 2011, Cancer Res, Vol. 71, p. 78-86. Also, “hsa-mir-2467” (miRBase Accession No. MI0017432, SEQ ID NO: 380) having a hairpin-like structure is known as a precursor of “hsa-miR-2467-3p”.

The term “hsa-miR-4728-5p gene” or “hsa-miR-4728-5p” used herein includes the hsa-miR-4728-5p gene (miRBase Accession No. MIMAT0019849) described in SEQ ID NO: 106, a homolog or an ortholog of a different organism species, and the like. The hsa-miR-4728-5p gene can be obtained by a method described in Persson H et al., 2011, Cancer Res, Vol. 71, p. 78-86. Also, “hsa-mir-4728” (miRBase Accession No. MI0017365, SEQ ID NO: 381) having a hairpin-like structure is known as a precursor of “hsa-miR-4728-5p”.

The term “hsa-miR-5572 gene” or “hsa-miR-5572” used herein includes the hsa-miR-5572 gene (miRBase Accession No. MIMAT0022260) described in SEQ ID NO: 107, a homolog or an ortholog of a different organism species, and the like. The hsa-miR-5572 gene can be obtained by a method described in Tandon M et al., 2012, Oral Dis, Vol. 18, p. 127-131. Also, “hsa-mir-5572” (miRBase Accession No. MI0019117, SEQ ID NO: 382) having a hairpin-like structure is known as a precursor of “hsa-miR-5572”.

The term “hsa-miR-6789-5p gene” or “hsa-miR-6789-5p” used herein includes the hsa-miR-6789-5p gene (miRBase Accession No. MIMAT0027478) described in SEQ ID NO: 108, a homolog or an ortholog of a different organism species, and the like. The hsa-miR-6789-5p gene can be obtained by a method described in Ladewig E et al., 2012, Genome Res, Vol. 22, p. 1634-1645. Also, “hsa-mir-6789” (miRBase Accession No. MI0022634, SEQ ID NO: 383) having a hairpin-like structure is known as a precursor of “hsa-miR-6789-5p”.

The term “hsa-miR-8063 gene” or “hsa-miR-8063” used herein includes the hsa-miR-8063 gene (miRBase Accession No. MIMAT0030990) described in SEQ ID NO: 109, a homolog or an ortholog of a different organism species, and the like. The hsa-miR-8063 gene can be obtained by a method described in Wang H J et al., 2013, Shock, Vol. 39, p. 480-487. Also, “hsa-mir-8063” (miRBase Accession No. MI0025899, SEQ ID NO: 384) having a hairpin-like structure is known as a precursor of “hsa-miR-8063”.

The term “hsa-miR-4429 gene” or “hsa-miR-4429” used herein includes the hsa-miR-4429 gene (miRBase Accession No. MIMAT0018944) described in SEQ ID NO: 110, a homolog or an ortholog of a different organism species, and the like. The hsa-miR-4429 gene can be obtained by a method described in Jima D D et al., 2010, Blood, Vol. 116, e118-e127. Also, “hsa-mir-4429” (miRBase Accession No. MI0016768, SEQ ID NO: 385) having a hairpin-like structure is known as a precursor of “hsa-miR-4429”.

The term “hsa-miR-6840-3p gene” or “hsa-miR-6840-3p” used herein includes the hsa-miR-6840-3p gene (miRBase Accession No. MIMAT0027583) described in SEQ ID NO: 111, a homolog or an ortholog of a different organism species, and the like. The hsa-miR-6840-3p gene can be obtained by a method described in Ladewig E et al., 2012, Genome Res, Vol. 22, p. 1634-1645. Also, “hsa-mir-6840” (miRBase Accession No. MI0022686, SEQ ID NO: 386) having a hairpin-like structure is known as a precursor of “hsa-miR-6840-3p”.

The term “hsa-miR-4476 gene” or “hsa-miR-4476” used herein includes the hsa-miR-4476 gene (miRBase Accession No. MIMAT0019003) described in SEQ ID NO: 112, a homolog or an ortholog of a different organism species, and the like. The hsa-miR-4476 gene can be obtained by a method described in Jima D D et al., 2010, Blood, Vol. 116, e118-e127. Also, “hsa-mir-4476” (miRBase Accession No. MI0016828, SEQ ID NO: 387) having a hairpin-like structure is known as a precursor of “hsa-miR-4476”.

The term “hsa-miR-675-5p gene” or “hsa-miR-675-5p” used herein includes the hsa-miR-675-5p gene (miRBase Accession No. MIMAT0004284) described in SEQ ID NO: 113, a homolog or an ortholog of a different organism species, and the like. The hsa-miR-675-5p gene can be obtained by a method described in Cai X et al., 2007, RNA, Vol. 13, p. 313-316. Also, “hsa-mir-675” (miRBase Accession No. MI0005416, SEQ ID NO: 388) having a hairpin-like structure is known as a precursor of “hsa-miR-675-5p”.

The term “hsa-miR-711 gene” or “hsa-miR-711” used herein includes the hsa-miR-711 gene (miRBase Accession No. MIMAT0012734) described in SEQ ID NO: 114, a homolog or an ortholog of a different organism species, and the like. The hsa-miR-711 gene can be obtained by a method described in Artzi S et al., 2008, BMC Bioinformatics, Vol. 9, p. 39. Also, “hsa-mir-711” (miRBase Accession No. MI0012488, SEQ ID NO: 389) having a hairpin-like structure is known as a precursor of “hsa-miR-711”.

The term “hsa-miR-6875-5p gene” or “hsa-miR-6875-5p” used herein includes the hsa-miR-6875-5p gene (miRBase Accession No. MIMAT0027650) described in SEQ ID NO: 115, a homolog or an ortholog of a different organism species, and the like. The hsa-miR-6875-5p gene can be obtained by a method described in Ladewig E et al., 2012, Genome Res, Vol. 22, p. 1634-1645. Also, “hsa-mir-6875” (miRBase Accession No. MI0022722, SEQ ID NO: 390) having a hairpin-like structure is known as a precursor of “hsa-miR-6875-5p”.

The term “hsa-miR-3160-5p gene” or “hsa-miR-3160-5p” used herein includes the hsa-miR-3160-5p gene (miRBase Accession No. MIMAT0019212) described in SEQ ID NO: 116, a homolog or an ortholog of a different organism species, and the like. The hsa-miR-3160-5p gene can be obtained by a method described in Stark M S et al., 2010, PLoS One, Vol. 5, e9685. Also, “hsa-mir-3160-1 and hsa-mir-3160-2” (miRBase Accession Nos. MI0014189 and MI0014190, SEQ ID NOs: 391 and 392) having a hairpin-like structure are known as precursors of “hsa-miR-3160-5p”.

The term “hsa-miR-1908-5p gene” or “hsa-miR-1908-5p” used herein includes the hsa-miR-1908-5p gene (miRBase Accession No. MIMAT0007881) described in SEQ ID NO: 117, a homolog or an ortholog of a different organism species, and the like. The hsa-miR-1908-5p gene can be obtained by a method described in Bar M et al., 2008, Stem Cells, Vol. 26, p. 2496-2505. Also, “hsa-mir-1908” (miRBase Accession No. MI0008329, SEQ ID NO: 393) having a hairpin-like structure is known as a precursor of “hsa-miR-1908-5p”.

The term “hsa-miR-6726-5p gene” or “hsa-miR-6726-5p” used herein includes the hsa-miR-6726-5p gene (miRBase Accession No. MIMAT0027353) described in SEQ ID NO: 118, a homolog or an ortholog of a different organism species, and the like. The hsa-miR-6726-5p gene can be obtained by a method described in Ladewig E et al., 2012, Genome Res, Vol. 22, p. 1634-1645. Also, “hsa-mir-6726” (miRBase Accession No. MI0022571, SEQ ID NO: 394) having a hairpin-like structure is known as a precursor of “hsa-miR-6726-5p”.

The term “hsa-miR-1913 gene” or “hsa-miR-1913” used herein includes the hsa-miR-1913 gene (miRBase Accession No. MIMAT0007888) described in SEQ ID NO: 119, a homolog or an ortholog of a different organism species, and the like. The hsa-miR-1913 gene can be obtained by a method described in Bar M et al., 2008, Stem Cells, Vol. 26, p. 2496-2505. Also, “hsa-mir-1913” (miRBase Accession No. MI0008334, SEQ ID NO: 395) having a hairpin-like structure is known as a precursor of “hsa-miR-1913”.

The term “hsa-miR-8071 gene” or “hsa-miR-8071” used herein includes the hsa-miR-8071 gene (miRBase Accession No. MIMAT0030998) described in SEQ ID NO: 120, a homolog or an ortholog of a different organism species, and the like. The hsa-miR-8071 gene can be obtained by a method described in Wang H J et al., 2013, Shock, Vol. 39, p. 480-487. Also, “hsa-mir-8071-1 and hsa-mir-8071-2” (miRBase Accession Nos. MI0025907 and MI0026417, SEQ ID NOs: 396 and 397) having a hairpin-like structure are known as precursors of “hsa-miR-8071”.

The term “hsa-miR-3648 gene” or “hsa-miR-3648” used herein includes the hsa-miR-3648 gene (miRBase Accession No. MIMAT0018068) described in SEQ ID NO: 121, a homolog or an ortholog of a different organism species, and the like. The hsa-miR-3648 gene can be obtained by a method described in Meiri E et al., 2010, Nucleic Acids Res, Vol. 38, p. 6234-6246. Also, “hsa-mir-3648-1 and hsa-mir-3648-2” (miRBase Accession Nos. MI0016048 and MI0031512, SEQ ID NOs: 398 and 399) having a hairpin-like structure are known as precursors of “hsa-miR-3648”.

The term “hsa-miR-4732-5p gene” or “hsa-miR-4732-5p” used herein includes the hsa-miR-4732-5p gene (miRBase Accession No. MIMAT0019855) described in SEQ ID NO: 122, a homolog or an ortholog of a different organism species, and the like. The hsa-miR-4732-5p gene can be obtained by a method described in Persson H et al., 2011, Cancer Res, Vol. 71, p. 78-86. Also, “hsa-mir-4732” (miRBase Accession No. MI0017369, SEQ ID NO: 400) having a hairpin-like structure is known as a precursor of “hsa-miR-4732-5p”.

The term “hsa-miR-4787-5p gene” or “hsa-miR-4787-5p” used herein includes the hsa-miR-4787-5p gene (miRBase Accession No. MIMAT0019956) described in SEQ ID NO: 123, a homolog or an ortholog of a different organism species, and the like. The hsa-miR-4787-5p gene can be obtained by a method described in Persson H et al., 2011, Cancer Res, Vol. 71, p. 78-86. Also, “hsa-mir-4787” (miRBase Accession No. MI0017434, SEQ ID NO: 318) having a hairpin-like structure is known as a precursor of “hsa-miR-4787-5p”.

The term “hsa-miR-3917 gene” or “hsa-miR-3917” used herein includes the hsa-miR-3917 gene (miRBase Accession No. MIMAT0018191) described in SEQ ID NO: 124, a homolog or an ortholog of a different organism species, and the like. The hsa-miR-3917 gene can be obtained by a method described in Creighton C J et al., 2010, PLoS One, Vol. 5, e9637. Also, “hsa-mir-3917” (miRBase Accession No. MI0016423, SEQ ID NO: 401) having a hairpin-like structure is known as a precursor of “hsa-miR-3917”.

The term “hsa-miR-619-5p gene” or “hsa-miR-619-5p” used herein includes the hsa-miR-619-5p gene (miRBase Accession No. MIMAT0026622) described in SEQ ID NO: 125, a homolog or an ortholog of a different organism species, and the like. The hsa-miR-619-5p gene can be obtained by a method described in Cummins J M et al., 2006, Proc Natl Acad Sci USA, Vol. 103, p. 3687-3692. Also, “hsa-mir-619” (miRBase Accession No. MI0003633, SEQ ID NO: 402) having a hairpin-like structure is known as a precursor of “hsa-miR-619-5p”.

The term “hsa-miR-1231 gene” or “hsa-miR-1231” used herein includes the hsa-miR-1231 gene (miRBase Accession No. MIMAT0005586) described in SEQ ID NO: 126, a homolog or an ortholog of a different organism species, and the like. The hsa-miR-1231 gene can be obtained by a method described in Berezikov E et al., 2007, Mol Cell, Vol. 28, p. 328-336. Also, “hsa-mir-1231” (miRBase Accession No. MI0006321, SEQ ID NO: 403) having a hairpin-like structure is known as a precursor of “hsa-miR-1231”.

The term “hsa-miR-342-5p gene” or “hsa-miR-342-5p” used herein includes the hsa-miR-342-5p gene (miRBase Accession No. MIMAT0004694) described in SEQ ID NO: 127, a homolog or an ortholog of a different organism species, and the like. The hsa-miR-342-5p gene can be obtained by a method described in Kim J et al., 2004, Proc Natl Acad Sci USA, Vol. 101, p. 360-365. Also, “hsa-mir-342” (miRBase Accession No. MI0000805, SEQ ID NO: 404) having a hairpin-like structure is known as a precursor of “hsa-miR-342-5p”.

The term “hsa-miR-4433a-5p gene” or “hsa-miR-4433a-5p” used herein includes the hsa-miR-4433a-5p gene (miRBase Accession No. MIMAT0020956) described in SEQ ID NO: 128, a homolog or an ortholog of a different organism species, and the like. The hsa-miR-4433a-5p gene can be obtained by a method described in Jima D D et al., 2010, Blood, Vol. 116, e118-e127. Also, “hsa-mir-4433a” (miRBase Accession No. MI0016773, SEQ ID NO: 405) having a hairpin-like structure is known as a precursor of “hsa-miR-4433a-5p”.

The term “hsa-miR-6766-5p gene” or “hsa-miR-6766-5p” used herein includes the hsa-miR-6766-5p gene (miRBase Accession No. MIMAT0027432) described in SEQ ID NO: 129, a homolog or an ortholog of a different organism species, and the like. The hsa-miR-6766-5p gene can be obtained by a method described in Ladewig E et al., 2012, Genome Res, Vol. 22, p. 1634-1645. Also, “hsa-mir-6766” (miRBase Accession No. MI0022611, SEQ ID NO: 365) having a hairpin-like structure is known as a precursor of “hsa-miR-6766-5p”.

The term “hsa-miR-4707-5p gene” or “hsa-miR-4707-5p” used herein includes the hsa-miR-4707-5p gene (miRBase Accession No. MIMAT0019807) described in SEQ ID NO: 130, a homolog or an ortholog of a different organism species, and the like. The hsa-miR-4707-5p gene can be obtained by a method described in Persson H et al., 2011, Cancer Res, Vol. 71, p. 78-86. Also, “hsa-mir-4707” (miRBase Accession No. MI0017340, SEQ ID NO: 406) having a hairpin-like structure is known as a precursor of “hsa-miR-4707-5p”.

The term “hsa-miR-7114-5p gene” or “hsa-miR-7114-5p” used herein includes the hsa-miR-7114-5p gene (miRBase Accession No. MIMAT0028125) described in SEQ ID NO: 131, a homolog or an ortholog of a different organism species, and the like. The hsa-miR-7114-5p gene can be obtained by a method described in Ladewig E et al., 2012, Genome Res, Vol. 22, p. 1634-1645. Also, “hsa-mir-7114” (miRBase Accession No. MI0022965, SEQ ID NO: 407) having a hairpin-like structure is known as a precursor of “hsa-miR-7114-5p”.

The term “hsa-miR-6872-3p gene” or “hsa-miR-6872-3p” used herein includes the hsa-miR-6872-3p gene (miRBase Accession No. MIMAT0027645) described in SEQ ID NO: 132, a homolog or an ortholog of a different organism species, and the like. The hsa-miR-6872-3p gene can be obtained by a method described in Ladewig E et al., 2012, Genome Res, Vol. 22, p. 1634-1645. Also, “hsa-mir-6872” (miRBase Accession No. MI0022719, SEQ ID NO: 408) having a hairpin-like structure is known as a precursor of “hsa-miR-6872-3p”.

The term “hsa-miR-6780b-5p gene” or “hsa-miR-6780b-5p” used herein includes the hsa-miR-6780b-5p gene (miRBase Accession No. MIMAT0027572) described in SEQ ID NO: 133, a homolog or an ortholog of a different organism species, and the like. The hsa-miR-6780b-5p gene can be obtained by a method described in Ladewig E et al., 2012, Genome Res, Vol. 22, p. 1634-1645. Also, “hsa-mir-6780b” (miRBase Accession No. MI0022681, SEQ ID NO: 409) having a hairpin-like structure is known as a precursor of “hsa-miR-6780b-5p”.

The term “hsa-miR-7845-5p gene” or “hsa-miR-7845-5p” used herein includes the hsa-miR-7845-5p gene (miRBase Accession No. MIMAT0030420) described in SEQ ID NO: 134, a homolog or an ortholog of a different organism species, and the like. The hsa-miR-7845-5p gene can be obtained by a method described in Ple H et al., 2012, PLoS One, Vol. 7, e50746. Also, “hsa-mir-7845” (miRBase Accession No. M10025515, SEQ ID NO: 410) having a hairpin-like structure is known as a precursor of “hsa-miR-7845-5p”.

The term “hsa-miR-6798-3p gene” or “hsa-miR-6798-3p” used herein includes the hsa-miR-6798-3p gene (miRBase Accession No. MIMAT0027497) described in SEQ ID NO: 135, a homolog or an ortholog of a different organism species, and the like. The hsa-miR-6798-3p gene can be obtained by a method described in Ladewig E et al., 2012, Genome Res, Vol. 22, p. 1634-1645. Also, “hsa-mir-6798” (miRBase Accession No. MI0022643, SEQ ID NO: 314) having a hairpin-like structure is known as a precursor of “hsa-miR-6798-3p”.

The term “hsa-miR-665 gene” or “hsa-miR-665” used herein includes the hsa-miR-665 gene (miRBase Accession No. MIMAT0004952) described in SEQ ID NO: 136, a homolog or an ortholog of a different organism species, and the like. The hsa-miR-665 gene can be obtained by a method described in Berezikov E et al., 2006, Genome Res, Vol. 16, p. 1289-1298. Also, “hsa-mir-665” (miRBase Accession No. MI0005563, SEQ ID NO: 411) having a hairpin-like structure is known as a precursor of “hsa-miR-665”.

The term “hsa-miR-6848-5p gene” or “hsa-miR-6848-5p” used herein includes the hsa-miR-6848-5p gene (miRBase Accession No. MIMAT0027596) described in SEQ ID NO: 137, a homolog or an ortholog of a different organism species, and the like. The hsa-miR-6848-5p gene can be obtained by a method described in Ladewig E et al., 2012, Genome Res, Vol. 22, p. 1634-1645. Also, “hsa-mir-6848” (miRBase Accession No. MI0022694, SEQ ID NO: 412) having a hairpin-like structure is known as a precursor of “hsa-miR-6848-5p”.

The term “hsa-miR-5008-5p gene” or “hsa-miR-5008-5p” used herein includes the hsa-miR-5008-5p gene (miRBase Accession No. MIMAT0021039) described in SEQ ID NO: 138, a homolog or an ortholog of a different organism species, and the like. The hsa-miR-5008-5p gene can be obtained by a method described in Hansen T B et al., 2011, RNA Biol, Vol. 8, p. 378-383. Also, “hsa-mir-5008” (miRBase Accession No. MI0017876, SEQ ID NO: 413) having a hairpin-like structure is known as a precursor of “hsa-miR-5008-5p”.

The term “hsa-miR-4294 gene” or “hsa-miR-4294” used herein includes the hsa-miR-4294 gene (miRBase Accession No. MIMAT0016849) described in SEQ ID NO: 139, a homolog or an ortholog of a different organism species, and the like. The hsa-miR-4294 gene can be obtained by a method described in Goff L A et al., 2009, PLoS One, Vol. 4, e7192. Also, “hsa-mir-4294” (miRBase Accession No. MI0015827, SEQ ID NO: 414) having a hairpin-like structure is known as a precursor of “hsa-miR-4294”.

The term “hsa-miR-6511a-5p gene” or “hsa-miR-6511a-5p” used herein includes the hsa-miR-6511a-5p gene (miRBase Accession No. MIMAT0025478) described in SEQ ID NO: 140, a homolog or an ortholog of a different organism species, and the like. The hsa-miR-6511a-5p gene can be obtained by a method described in Joyce C E et al., 2011, Hum Mol Genet, Vol. 20, p. 4025-4040. Also, “hsa-mir-6511a-1, hsa-mir-6511a-2, hsa-mir-6511a-3 and hsa-mir-6511a-4” (miRBase Accession Nos. MI0022223, MI0023564, MI0023565 and MI0023566, SEQ ID NOs: 415, 416, 417 and 418) having a hairpin-like structure are known as precursors of “hsa-miR-6511a-5p”.

The term “hsa-miR-4435 gene” or “hsa-miR-4435” used herein includes the hsa-miR-4435 gene (miRBase Accession No. MIMAT0018951) described in SEQ ID NO: 141, a homolog or an ortholog of a different organism species, and the like. The hsa-miR-4435 gene can be obtained by a method described in Jima D D et al., 2010, Blood, Vol. 116, e118-e127. Also, “hsa-mir-4435-1 and hsa-mir-4435-2” (miRBase Accession Nos. MI0016775 and MI0016777, SEQ ID NOs: 419 and 420) having a hairpin-like structure are known as precursors of “hsa-miR-4435”.

The term “hsa-miR-4747-3p gene” or “hsa-miR-4747-3p” used herein includes the hsa-miR-4747-3p gene (miRBase Accession No. MIMAT0019883) described in SEQ ID NO: 142, a homolog or an ortholog of a different organism species, and the like. The hsa-miR-4747-3p gene can be obtained by a method described in Persson H et al., 2011, Cancer Res, Vol. 71, p. 78-86. Also, “hsa-mir-4747” (miRBase Accession No. MI0017386, SEQ ID NO: 421) having a hairpin-like structure is known as a precursor of “hsa-miR-4747-3p”.

The term “hsa-miR-6880-3p gene” or “hsa-miR-6880-3p” used herein includes the hsa-miR-6880-3p gene (miRBase Accession No. MIMAT0027661) described in SEQ ID NO: 143, a homolog or an ortholog of a different organism species, and the like. The hsa-miR-6880-3p gene can be obtained by a method described in Ladewig E et al., 2012, Genome Res, Vol. 22, p. 1634-1645. Also, “hsa-mir-6880” (miRBase Accession No. MI0022727, SEQ ID NO: 422) having a hairpin-like structure is known as a precursor of “hsa-miR-6880-3p”.

The term “hsa-miR-6869-5p gene” or “hsa-miR-6869-5p” used herein includes the hsa-miR-6869-5p gene (miRBase Accession No. MIMAT0027638) described in SEQ ID NO: 144, a homolog or an ortholog of a different organism species, and the like. The hsa-miR-6869-5p gene can be obtained by a method described in Ladewig E et al., 2012, Genome Res, Vol. 22, p. 1634-1645. Also, “hsa-mir-6869” (miRBase Accession No. MI0022716, SEQ ID NO: 423) having a hairpin-like structure is known as a precursor of “hsa-miR-6869-5p”.

The term “hsa-miR-7150 gene” or “hsa-miR-7150” used herein includes the hsa-miR-7150 gene (miRBase Accession No. MIMAT0028211) described in SEQ ID NO: 145, a homolog or an ortholog of a different organism species, and the like. The hsa-miR-7150 gene can be obtained by a method described in Oulas A et al., 2009, Nucleic Acids Res, Vol. 37, p. 3276-3287. Also, “hsa-mir-7150” (miRBase Accession No. MI0023610, SEQ ID NO: 424) having a hairpin-like structure is known as a precursor of “hsa-miR-7150”.

The term “hsa-miR-1260a gene” or “hsa-miR-1260a” used herein includes the hsa-miR-1260a gene (miRBase Accession No. MIMAT0005911) described in SEQ ID NO: 146, a homolog or an ortholog of a different organism species, and the like. The hsa-miR-1260a gene can be obtained by a method described in Morin R D et al., 2008, Genome Res, Vol. 18, p. 610-621. Also, “hsa-mir-1260a” (miRBase Accession No. MI0006394, SEQ ID NO: 425) having a hairpin-like structure is known as a precursor of “hsa-miR-1260a”.

The term “hsa-miR-6877-5p gene” or “hsa-miR-6877-5p” used herein includes the hsa-miR-6877-5p gene (miRBase Accession No. MIMAT0027654) described in SEQ ID NO: 147, a homolog or an ortholog of a different organism species, and the like. The hsa-miR-6877-5p gene can be obtained by a method described in Ladewig E et al., 2012, Genome Res, Vol. 22, p. 1634-1645. Also, “hsa-mir-6877” (miRBase Accession No. MI0022724, SEQ ID NO: 426) having a hairpin-like structure is known as a precursor of “hsa-miR-6877-5p”.

The term “hsa-miR-6721-5p gene” or “hsa-miR-6721-5p” used herein includes the hsa-miR-6721-5p gene (miRBase Accession No. MIMAT0025852) described in SEQ ID NO: 148, a homolog or an ortholog of a different organism species, and the like. The hsa-miR-6721-5p gene can be obtained by a method described in Li Y et al., 2012, Gene, Vol. 497, p. 330-335. Also, “hsa-mir-6721” (miRBase Accession No. MI0022556, SEQ ID NO: 427) having a hairpin-like structure is known as a precursor of “hsa-miR-6721-5p”.

The term “hsa-miR-4656 gene” or “hsa-miR-4656” used herein includes the hsa-miR-4656 gene (miRBase Accession No. MIMAT0019723) described in SEQ ID NO: 149, a homolog or an ortholog of a different organism species, and the like. The hsa-miR-4656 gene can be obtained by a method described in Persson H et al., 2011, Cancer Res, Vol. 71, p. 78-86. Also, “hsa-mir-4656” (miRBase Accession No. MI0017284, SEQ ID NO: 428) having a hairpin-like structure is known as a precursor of “hsa-miR-4656”.

The term “hsa-miR-1229-5p gene” or “hsa-miR-1229-5p” used herein includes the hsa-miR-1229-5p gene (miRBase Accession No. MIMAT0022942) described in SEQ ID NO: 150, a homolog or an ortholog of a different organism species, and the like. The hsa-miR-1229-5p gene can be obtained by a method described in Berezikov E et al., 2007, Mol Cell, Vol. 28, p. 328-336. Also, “hsa-mir-1229” (miRBase Accession No. MI0006319, SEQ ID NO: 429) having a hairpin-like structure is known as a precursor of “hsa-miR-1229-5p”.

The term “hsa-miR-4433a-3p gene” or “hsa-miR-4433a-3p” used herein includes the hsa-miR-4433a-3p gene (miRBase Accession No. MIMAT0018949) described in SEQ ID NO: 151, a homolog or an ortholog of a different organism species, and the like. The hsa-miR-4433a-3p gene can be obtained by a method described in Jima D D et al., 2010, Blood, Vol. 116, e118-e127. Also, “hsa-mir-4433a” (miRBase Accession No. MI0016773, SEQ ID NO: 405) having a hairpin-like structure is known as a precursor of “hsa-miR-4433a-3p”.

The term “hsa-miR-4274 gene” or “hsa-miR-4274” used herein includes the hsa-miR-4274 gene (miRBase Accession No. MIMAT0016906) described in SEQ ID NO: 152, a homolog or an ortholog of a different organism species, and the like. The hsa-miR-4274 gene can be obtained by a method described in Goff L A et al., 2009, PLoS One, Vol. 4, e7192. Also, “hsa-mir-4274” (miRBase Accession No. MI0015884, SEQ ID NO: 430) having a hairpin-like structure is known as a precursor of “hsa-miR-4274”.

The term “hsa-miR-4419b gene” or “hsa-miR-4419b” used herein includes the hsa-miR-4419b gene (miRBase Accession No. MIMAT0019034) described in SEQ ID NO: 153, a homolog or an ortholog of a different organism species, and the like. The hsa-miR-4419b gene can be obtained by a method described in Jima D D et al., 2010, Blood, Vol. 116, e118-e127. Also, “hsa-mir-4419b” (miRBase Accession No. MI0016861, SEQ ID NO: 431) having a hairpin-like structure is known as a precursor of “hsa-miR-4419b”.

The term “hsa-miR-4674 gene” or “hsa-miR-4674” used herein includes the hsa-miR-4674 gene (miRBase Accession No. MIMAT0019756) described in SEQ ID NO: 154, a homolog or an ortholog of a different organism species, and the like. The hsa-miR-4674 gene can be obtained by a method described in Persson H et al., 2011, Cancer Res, Vol. 71, p. 78-86. Also, “hsa-mir-4674” (miRBase Accession No. MI0017305, SEQ ID NO: 432) having a hairpin-like structure is known as a precursor of “hsa-miR-4674”.

The term “hsa-miR-6893-5p gene” or “hsa-miR-6893-5p” used herein includes the hsa-miR-6893-5p gene (miRBase Accession No. MIMAT0027686) described in SEQ ID NO: 155, a homolog or an ortholog of a different organism species, and the like. The hsa-miR-6893-5p gene can be obtained by a method described in Ladewig E et al., 2012, Genome Res, Vol. 22, p. 1634-1645. Also, “hsa-mir-6893” (miRBase Accession No. MI0022740, SEQ ID NO: 433) having a hairpin-like structure is known as a precursor of “hsa-miR-6893-5p”.

The term “hsa-miR-6763-3p gene” or “hsa-miR-6763-3p” used herein includes the hsa-miR-6763-3p gene (miRBase Accession No. MIMAT0027427) described in SEQ ID NO: 156, a homolog or an ortholog of a different organism species, and the like. The hsa-miR-6763-3p gene can be obtained by a method described in Ladewig E et al., 2012, Genome Res, Vol. 22, p. 1634-1645. Also, “hsa-mir-6763” (miRBase Accession No. MI0022608, SEQ ID NO: 434) having a hairpin-like structure is known as a precursor of “hsa-miR-6763-3p”.

The term “hsa-miR-6762-5p gene” or “hsa-miR-6762-5p” used herein includes the hsa-miR-6762-5p gene (miRBase Accession No. MIMAT0027424) described in SEQ ID NO: 157, a homolog or an ortholog of a different organism species, and the like. The hsa-miR-6762-5p gene can be obtained by a method described in Ladewig E et al., 2012, Genome Res, Vol. 22, p. 1634-1645. Also, “hsa-mir-6762” (miRBase Accession No. MI0022607, SEQ ID NO: 435) having a hairpin-like structure is known as a precursor of “hsa-miR-6762-5p”.

The term “hsa-miR-6738-5p gene” or “hsa-miR-6738-5p” used herein includes the hsa-miR-6738-5p gene (miRBase Accession No. MIMAT0027377) described in SEQ ID NO: 158, a homolog or an ortholog of a different organism species, and the like. The hsa-miR-6738-5p gene can be obtained by a method described in Ladewig E et al., 2012, Genome Res, Vol. 22, p. 1634-1645. Also, “hsa-mir-6738” (miRBase Accession No. MI0022583, SEQ ID NO: 436) having a hairpin-like structure is known as a precursor of “hsa-miR-6738-5p”.

The term “hsa-miR-4513 gene” or “hsa-miR-4513” used herein includes the hsa-miR-4513 gene (miRBase Accession No. MIMAT0019050) described in SEQ ID NO: 159, a homolog or an ortholog of a different organism species, and the like. The hsa-miR-4513 gene can be obtained by a method described in Jima D D et al., 2010, Blood, Vol. 116, e118-e127. Also, “hsa-mir-4513” (miRBase Accession No. MI0016879, SEQ ID NO: 437) having a hairpin-like structure is known as a precursor of “hsa-miR-4513”.

The term “hsa-miR-6746-5p gene” or “hsa-miR-6746-5p” used herein includes the hsa-miR-6746-5p gene (miRBase Accession No. MIMAT0027392) described in SEQ ID NO: 160, a homolog or an ortholog of a different organism species, and the like. The hsa-miR-6746-5p gene can be obtained by a method described in Ladewig E et al., 2012, Genome Res, Vol. 22, p. 1634-1645. Also, “hsa-mir-6746” (miRBase Accession No. MI0022591, SEQ ID NO: 438) having a hairpin-like structure is known as a precursor of “hsa-miR-6746-5p”.

The term “hsa-miR-6880-5p gene” or “hsa-miR-6880-5p” used herein includes the hsa-miR-6880-5p gene (miRBase Accession No. MIMAT0027660) described in SEQ ID NO: 161, a homolog or an ortholog of a different organism species, and the like. The hsa-miR-6880-5p gene can be obtained by a method described in Ladewig E et al., 2012, Genome Res, Vol. 22, p. 1634-1645. Also, “hsa-mir-6880” (miRBase Accession No. MI0022727, SEQ ID NO: 422) having a hairpin-like structure is known as a precursor of “hsa-miR-6880-5p”.

The term “hsa-miR-4736 gene” or “hsa-miR-4736” used herein includes the hsa-miR-4736 gene (miRBase Accession No. MIMAT0019862) described in SEQ ID NO: 162, a homolog or an ortholog of a different organism species, and the like. The hsa-miR-4736 gene can be obtained by a method described in Persson H et al., 2011, Cancer Res, Vol. 71, p. 78-86. Also, “hsa-mir-4736” (miRBase Accession No. MI0017373, SEQ ID NO: 439) having a hairpin-like structure is known as a precursor of “hsa-miR-4736”.

The term “hsa-miR-718 gene” or “hsa-miR-718” used herein includes the hsa-miR-718 gene (miRBase Accession No. MIMAT0012735) described in SEQ ID NO: 163, a homolog or an ortholog of a different organism species, and the like. The hsa-miR-718 gene can be obtained by a method described in Artzi S et al., 2008, BMC Bioinformatics, Vol. 9, p. 39. Also, “hsa-mir-718” (miRBase Accession No. MI0012489, SEQ ID NO: 440) having a hairpin-like structure is known as a precursor of “hsa-miR-718”.

The term “hsa-miR-6717-5p gene” or “hsa-miR-6717-5p” used herein includes the hsa-miR-6717-5p gene (miRBase Accession No. MIMAT0025846) described in SEQ ID NO: 164, a homolog or an ortholog of a different organism species, and the like. The hsa-miR-6717-5p gene can be obtained by a method described in Li Y et al., 2012, Gene, Vol. 497, p. 330-335. Also, “hsa-mir-6717” (miRBase Accession No. M10022551, SEQ ID NO: 441) having a hairpin-like structure is known as a precursor of “hsa-miR-6717-5p”.

The term “hsa-miR-7847-3p gene” or “hsa-miR-7847-3p” used herein includes the hsa-miR-7847-3p gene (miRBase Accession No. MIMAT0030422) described in SEQ ID NO: 165, a homolog or an ortholog of a different organism species, and the like. The hsa-miR-7847-3p gene can be obtained by a method described in Ple H et al., 2012, PLoS One, Vol. 7, e50746. Also, “hsa-mir-7847” (miRBase Accession No. M10025517, SEQ ID NO: 442) having a hairpin-like structure is known as a precursor of “hsa-miR-7847-3p”.

The term “hsa-miR-760 gene” or “hsa-miR-760” used herein includes the hsa-miR-760 gene (miRBase Accession No. MIMAT0004957) described in SEQ ID NO: 166, a homolog or an ortholog of a different organism species, and the like. The hsa-miR-760 gene can be obtained by a method described in Berezikov E et al., 2006, Genome Res, Vol. 16, p. 1289-1298. Also, “hsa-mir-760” (miRBase Accession No. MI0005567, SEQ ID NO: 443) having a hairpin-like structure is known as a precursor of “hsa-miR-760”.

The term “hsa-miR-1199-5p gene” or “hsa-miR-1199-5p” used herein includes the hsa-miR-1199-5p gene (miRBase Accession No. MIMAT0031119) described in SEQ ID NO: 167, a homolog or an ortholog of a different organism species, and the like. The hsa-miR-1199-5p gene can be obtained by a method described in Salvi A et al., 2013, Int J Oncol, Vol. 42, p. 391-402. Also, “hsa-mir-1199” (miRBase Accession No. MI0020340, SEQ ID NO: 444) having a hairpin-like structure is known as a precursor of “hsa-miR-1199-5p”.

The term “hsa-miR-6813-5p gene” or “hsa-miR-6813-5p” used herein includes the hsa-miR-6813-5p gene (miRBase Accession No. MIMAT0027526) described in SEQ ID NO: 168, a homolog or an ortholog of a different organism species, and the like. The hsa-miR-6813-5p gene can be obtained by a method described in Ladewig E et al., 2012, Genome Res, Vol. 22, p. 1634-1645. Also, “hsa-mir-6813” (miRBase Accession No. MI0022658, SEQ ID NO: 445) having a hairpin-like structure is known as a precursor of “hsa-miR-6813-5p”.

The term “hsa-miR-6769a-5p gene” or “hsa-miR-6769a-5p” used herein includes the hsa-miR-6769a-5p gene (miRBase Accession No. MIMAT0027438) described in SEQ ID NO: 169, a homolog or an ortholog of a different organism species, and the like. The hsa-miR-6769a-5p gene can be obtained by a method described in Ladewig E et al., 2012, Genome Res, Vol. 22, p. 1634-1645. Also, “hsa-mir-6769a” (miRBase Accession No. MI0022614, SEQ ID NO: 446) having a hairpin-like structure is known as a precursor of “hsa-miR-6769a-5p”.

The term “hsa-miR-1193 gene” or “hsa-miR-1193” used herein includes the hsa-miR-1193 gene (miRBase Accession No. MIMAT0015049) described in SEQ ID NO: 170, a homolog or an ortholog of a different organism species, and the like. The hsa-miR-1193 gene can be obtained by a method described in Stark M S et al., 2010, PLoS One, Vol. 5, e9685. Also, “hsa-mir-1193” (miRBase Accession No. MI0014205, SEQ ID NO: 447) having a hairpin-like structure is known as a precursor of “hsa-miR-1193”.

The term “hsa-miR-7108-3p gene” or “hsa-miR-7108-3p” used herein includes the hsa-miR-7108-3p gene (miRBase Accession No. MIMAT0028114) described in SEQ ID NO: 171, a homolog or an ortholog of a different organism species, and the like. The hsa-miR-7108-3p gene can be obtained by a method described in Ladewig E et al., 2012, Genome Res, Vol. 22, p. 1634-1645. Also, “hsa-mir-7108” (miRBase Accession No. MI0022959, SEQ ID NO: 448) having a hairpin-like structure is known as a precursor of “hsa-miR-7108-3p”.

The term “hsa-miR-6741-5p gene” or “hsa-miR-6741-5p” used herein includes the hsa-miR-6741-5p gene (miRBase Accession No. MIMAT0027383) described in SEQ ID NO: 172, a homolog or an ortholog of a different organism species, and the like. The hsa-miR-6741-5p gene can be obtained by a method described in Ladewig E et al., 2012, Genome Res, Vol. 22, p. 1634-1645. Also, “hsa-mir-6741” (miRBase Accession No. MI0022586, SEQ ID NO: 449) having a hairpin-like structure is known as a precursor of “hsa-miR-6741-5p”.

The term “hsa-miR-4298 gene” or “hsa-miR-4298” used herein includes the hsa-miR-4298 gene (miRBase Accession No. MIMAT0016852) described in SEQ ID NO: 173, a homolog or an ortholog of a different organism species, and the like. The hsa-miR-4298 gene can be obtained by a method described in Goff L A et al., 2009, PLoS One, Vol. 4, e7192. Also, “hsa-mir-4298” (miRBase Accession No. MI0015830, SEQ ID NO: 450) having a hairpin-like structure is known as a precursor of “hsa-miR-4298”.

The term “hsa-miR-6796-3p gene” or “hsa-miR-6796-3p” used herein includes the hsa-miR-6796-3p gene (miRBase Accession No. MIMAT0027493) described in SEQ ID NO: 174, a homolog or an ortholog of a different organism species, and the like. The hsa-miR-6796-3p gene can be obtained by a method described in Ladewig E et al., 2012, Genome Res, Vol. 22, p. 1634-1645. Also, “hsa-mir-6796” (miRBase Accession No. MI0022641, SEQ ID NO: 451) having a hairpin-like structure is known as a precursor of “hsa-miR-6796-3p”.

The term “hsa-miR-4750-5p gene” or “hsa-miR-4750-5p” used herein includes the hsa-miR-4750-5p gene (miRBase Accession No. MIMAT0019887) described in SEQ ID NO: 175, a homolog or an ortholog of a different organism species, and the like. The hsa-miR-4750-5p gene can be obtained by a method described in Persson H et al., 2011, Cancer Res, Vol. 71, p. 78-86. Also, “hsa-mir-4750” (miRBase Accession No. MI0017389, SEQ ID NO: 452) having a hairpin-like structure is known as a precursor of “hsa-miR-4750-5p”.

The term “hsa-miR-6785-5p gene” or “hsa-miR-6785-5p” used herein includes the hsa-miR-6785-5p gene (miRBase Accession No. MIMAT0027470) described in SEQ ID NO: 176, a homolog or an ortholog of a different organism species, and the like. The hsa-miR-6785-5p gene can be obtained by a method described in Ladewig E et al., 2012, Genome Res, Vol. 22, p. 1634-1645. Also, “hsa-mir-6785” (miRBase Accession No. MI0022630, SEQ ID NO: 453) having a hairpin-like structure is known as a precursor of “hsa-miR-6785-5p”.

The term “hsa-miR-1292-3p gene” or “hsa-miR-1292-3p” used herein includes the hsa-miR-1292-3p gene (miRBase Accession No. MIMAT0022948) described in SEQ ID NO: 177, a homolog or an ortholog of a different organism species, and the like. The hsa-miR-1292-3p gene can be obtained by a method described in Morin R D et al., 2008, Genome Res, Vol. 18, p. 610-621. Also, “hsa-mir-1292” (miRBase Accession No. MI0006433, SEQ ID NO: 454) having a hairpin-like structure is known as a precursor of “hsa-miR-1292-3p”.

The term “hsa-miR-4749-3p gene” or “hsa-miR-4749-3p” used herein includes the hsa-miR-4749-3p gene (miRBase Accession No. MIMAT0019886) described in SEQ ID NO: 178, a homolog or an ortholog of a different organism species, and the like. The hsa-miR-4749-3p gene can be obtained by a method described in Persson H et al., 2011, Cancer Res, Vol. 71, p. 78-86. Also, “hsa-mir-4749” (miRBase Accession No. MI0017388, SEQ ID NO: 455) having a hairpin-like structure is known as a precursor of “hsa-miR-4749-3p”.

The term “hsa-miR-6800-3p gene” or “hsa-miR-6800-3p” used herein includes the hsa-miR-6800-3p gene (miRBase Accession No. MIMAT0027501) described in SEQ ID NO: 179, a homolog or an ortholog of a different organism species, and the like. The hsa-miR-6800-3p gene can be obtained by a method described in Ladewig E et al., 2012, Genome Res, Vol. 22, p. 1634-1645. Also, “hsa-mir-6800” (miRBase Accession No. MI0022645, SEQ ID NO: 293) having a hairpin-like structure is known as a precursor of “hsa-miR-6800-3p”.

The term “hsa-miR-4722-5p gene” or “hsa-miR-4722-5p” used herein includes the hsa-miR-4722-5p gene (miRBase Accession No. MIMAT0019836) described in SEQ ID NO: 180, a homolog or an ortholog of a different organism species, and the like. The hsa-miR-4722-5p gene can be obtained by a method described in Persson H et al., 2011, Cancer Res, Vol. 71, p. 78-86. Also, “hsa-mir-4722” (miRBase Accession No. MI0017357, SEQ ID NO: 456) having a hairpin-like structure is known as a precursor of “hsa-miR-4722-5p”.

The term “hsa-miR-4746-3p gene” or “hsa-miR-4746-3p” used herein includes the hsa-miR-4746-3p gene (miRBase Accession No. MIMAT0019881) described in SEQ ID NO: 181, a homolog or an ortholog of a different organism species, and the like. The hsa-miR-4746-3p gene can be obtained by a method described in Persson H et al., 2011, Cancer Res, Vol. 71, p. 78-86. Also, “hsa-mir-4746” (miRBase Accession No. MI0017385, SEQ ID NO: 457) having a hairpin-like structure is known as a precursor of “hsa-miR-4746-3p”.

The term “hsa-miR-4450 gene” or “hsa-miR-4450” used herein includes the hsa-miR-4450 gene (miRBase Accession No. MIMAT0018971) described in SEQ ID NO: 182, a homolog or an ortholog of a different organism species, and the like. The hsa-miR-4450 gene can be obtained by a method described in Jima D D et al., 2010, Blood, Vol. 116, e118-e127. Also, “hsa-mir-4450” (miRBase Accession No. MI0016795, SEQ ID NO: 458) having a hairpin-like structure is known as a precursor of “hsa-miR-4450”.

The term “hsa-miR-6795-5p gene” or “hsa-miR-6795-5p” used herein includes the hsa-miR-6795-5p gene (miRBase Accession No. MIMAT0027490) described in SEQ ID NO: 183, a homolog or an ortholog of a different organism species, and the like. The hsa-miR-6795-5p gene can be obtained by a method described in Ladewig E et al., 2012, Genome Res, Vol. 22, p. 1634-1645. Also, “hsa-mir-6795” (miRBase Accession No. MI0022640, SEQ ID NO: 459) having a hairpin-like structure is known as a precursor of “hsa-miR-6795-5p”.

The term “hsa-miR-365a-5p gene” or “hsa-miR-365a-5p” used herein includes the hsa-miR-365a-5p gene (miRBase Accession No. MIMAT0009199) described in SEQ ID NO: 184, a homolog or an ortholog of a different organism species, and the like. The hsa-miR-365a-5p gene can be obtained by a method described in Xie X et al., 2005, Nature, Vol. 434, p. 338-345. Also, “hsa-mir-365a” (miRBase Accession No. MI0000767, SEQ ID NO: 460) having a hairpin-like structure is known as a precursor of “hsa-miR-365a-5p”.

The term “hsa-miR-498 gene” or “hsa-miR-498” used herein includes the hsa-miR-498 gene (miRBase Accession No. MIMAT0002824) described in SEQ ID NO: 185, a homolog or an ortholog of a different organism species, and the like. The hsa-miR-498 gene can be obtained by a method described in Bentwich I et al., 2005, Nat Genet, Vol. 37, p. 766-770. Also, “hsa-mir-498” (miRBase Accession No. MI0003142, SEQ ID NO: 461) having a hairpin-like structure is known as a precursor of “hsa-miR-498”.

The term “hsa-miR-6797-5p gene” or “hsa-miR-6797-5p” used herein includes the hsa-miR-6797-5p gene (miRBase Accession No. MIMAT0027494) described in SEQ ID NO: 186, a homolog or an ortholog of a different organism species, and the like. The hsa-miR-6797-5p gene can be obtained by a method described in Ladewig E et al., 2012, Genome Res, Vol. 22, p. 1634-1645. Also, “hsa-mir-6797” (miRBase Accession No. MI0022642, SEQ ID NO: 462) having a hairpin-like structure is known as a precursor of “hsa-miR-6797-5p”.

The term “hsa-miR-1470 gene” or “hsa-miR-1470” used herein includes the hsa-miR-1470 gene (miRBase Accession No. MIMAT0007348) described in SEQ ID NO: 187, a homolog or an ortholog of a different organism species, and the like. The hsa-miR-1470 gene can be obtained by a method described in Kawaji H et al., 2008, BMC Genomics, Vol. 9, p. 157. Also, “hsa-mir-1470” (miRBase Accession No. MI0007075, SEQ ID NO: 463) having a hairpin-like structure is known as a precursor of “hsa-miR-1470”.

The term “hsa-miR-6851-5p gene” or “hsa-miR-6851-5p” used herein includes the hsa-miR-6851-5p gene (miRBase Accession No. MIMAT0027602) described in SEQ ID NO: 188, a homolog or an ortholog of a different organism species, and the like. The hsa-miR-6851-5p gene can be obtained by a method described in Ladewig E et al., 2012, Genome Res, Vol. 22, p. 1634-1645. Also, “hsa-mir-6851” (miRBase Accession No. MI0022697, SEQ ID NO: 464) having a hairpin-like structure is known as a precursor of “hsa-miR-6851-5p”.

The term “hsa-miR-1247-3p gene” or “hsa-miR-1247-3p” used herein includes the hsa-miR-1247-3p gene (miRBase Accession No. MIMAT0022721) described in SEQ ID NO: 189, a homolog or an ortholog of a different organism species, and the like. The hsa-miR-1247-3p gene can be obtained by a method described in Morin R D et al., 2008, Genome Res, Vol. 18, p. 610-621. Also, “hsa-mir-1247” (miRBase Accession No. MI0006382, SEQ ID NO: 465) having a hairpin-like structure is known as a precursor of “hsa-miR-1247-3p”.

The term “hsa-miR-5196-5p gene” or “hsa-miR-5196-5p” used herein includes the hsa-miR-5196-5p gene (miRBase Accession No. MIMAT0021128) described in SEQ ID NO: 190, a homolog or an ortholog of a different organism species, and the like. The hsa-miR-5196-5p gene can be obtained by a method described in Schotte D et al., 2011, Leukemia, Vol. 25, p. 1389-1399. Also, “hsa-mir-5196” (miRBase Accession No. MI0018175, SEQ ID NO: 466) having a hairpin-like structure is known as a precursor of “hsa-miR-5196-5p”.

The term “hsa-miR-208a-5p gene” or “hsa-miR-208a-5p” used herein includes the hsa-miR-208a-5p gene (miRBase Accession No. MIMAT0026474) described in SEQ ID NO: 191, a homolog or an ortholog of a different organism species, and the like. The hsa-miR-208a-5p gene can be obtained by a method described in Lagos-Quintana M et al., 2003, RNA, Vol. 9, p. 175-179. Also, “hsa-mir-208a” (miRBase Accession No. MI0000251, SEQ ID NO: 467) having a hairpin-like structure is known as a precursor of “hsa-miR-208a-5p”.

The term “hsa-miR-6842-5p gene” or “hsa-miR-6842-5p” used herein includes the hsa-miR-6842-5p gene (miRBase Accession No. MIMAT0027586) described in SEQ ID NO: 192, a homolog or an ortholog of a different organism species, and the like. The hsa-miR-6842-5p gene can be obtained by a method described in Ladewig E et al., 2012, Genome Res, Vol. 22, p. 1634-1645. Also, “hsa-mir-6842” (miRBase Accession No. MI0022688, SEQ ID NO: 468) having a hairpin-like structure is known as a precursor of “hsa-miR-6842-5p”.

The term “hsa-miR-150-3p gene” or “hsa-miR-150-3p” used herein includes the hsa-miR-150-3p gene (miRBase Accession No. MIMAT0004610) described in SEQ ID NO: 193, a homolog or an ortholog of a different organism species, and the like. The hsa-miR-150-3p gene can be obtained by a method described in Lagos-Quintana M et al., 2002, Curr Biol, Vol. 12, p. 735-739. Also, “hsa-mir-150” (miRBase Accession No. MI0000479, SEQ ID NO: 469) having a hairpin-like structure is known as a precursor of “hsa-miR-150-3p”.

The term “hsa-miR-4534 gene” or “hsa-miR-4534” used herein includes the hsa-miR-4534 gene (miRBase Accession No. MIMAT0019073) described in SEQ ID NO: 194, a homolog or an ortholog of a different organism species, and the like. The hsa-miR-4534 gene can be obtained by a method described in Jima D D et al., 2010, Blood, Vol. 116, e118-e127. Also, “hsa-mir-4534” (miRBase Accession No. MI0016901, SEQ ID NO: 470) having a hairpin-like structure is known as a precursor of “hsa-miR-4534”.

The term “hsa-miR-3135b gene” or “hsa-miR-3135b” used herein includes the hsa-miR-3135b gene (miRBase Accession No. MIMAT0018985) described in SEQ ID NO: 195, a homolog or an ortholog of a different organism species, and the like. The hsa-miR-3135b gene can be obtained by a method described in Jima D D et al., 2010, Blood, Vol. 116, e118-e127. Also, “hsa-mir-3135b” (miRBase Accession No. MI0016809, SEQ ID NO: 471) having a hairpin-like structure is known as a precursor of “hsa-miR-3135b”.

The term “hsa-miR-3131 gene” or “hsa-miR-3131” used herein includes the hsa-miR-3131 gene (miRBase Accession No. MIMAT0014996) described in SEQ ID NO: 196, a homolog or an ortholog of a different organism species, and the like. The hsa-miR-3131 gene can be obtained by a method described in Stark M S et al., 2010, PLoS One, Vol. 5, e9685. Also, “hsa-mir-3131” (miRBase Accession No. MI0014151, SEQ ID NO: 472) having a hairpin-like structure is known as a precursor of “hsa-miR-3131”.

The term “hsa-miR-4792 gene” or “hsa-miR-4792” used herein includes the hsa-miR-4792 gene (miRBase Accession No. MIMAT0019964) described in SEQ ID NO: 197, a homolog or an ortholog of a different organism species, and the like. The hsa-miR-4792 gene can be obtained by a method described in Persson H et al., 2011, Cancer Res, Vol. 71, p. 78-86. Also, “hsa-mir-4792” (miRBase Accession No. MI0017439, SEQ ID NO: 473) having a hairpin-like structure is known as a precursor of “hsa-miR-4792”.

The term “hsa-miR-6510-5p gene” or “hsa-miR-6510-5p” used herein includes the hsa-miR-6510-5p gene (miRBase Accession No. MIMAT0025476) described in SEQ ID NO: 198, a homolog or an ortholog of a different organism species, and the like. The hsa-miR-6510-5p gene can be obtained by a method described in Joyce C E et al., 2011, Hum Mol Genet, Vol. 20, p. 4025-4040. Also, “hsa-mir-6510” (miRBase Accession No. MI0022222, SEQ ID NO: 474) having a hairpin-like structure is known as a precursor of “hsa-miR-6510-5p”.

The term “hsa-miR-504-3p gene” or “hsa-miR-504-3p” used herein includes the hsa-miR-504-3p gene (miRBase Accession No. MIMAT0026612) described in SEQ ID NO: 199, a homolog or an ortholog of a different organism species, and the like. The hsa-miR-504-3p gene can be obtained by a method described in Bentwich I et al., 2005, Nat Genet, Vol. 37, p. 766-770. Also, “hsa-mir-504” (miRBase Accession No. MI0003189, SEQ ID NO: 475) having a hairpin-like structure is known as a precursor of “hsa-miR-504-3p”.

The term “hsa-miR-3619-3p gene” or “hsa-miR-3619-3p” used herein includes the hsa-miR-3619-3p gene (miRBase Accession No. MIMAT0019219) described in SEQ ID NO: 200, a homolog or an ortholog of a different organism species, and the like. The hsa-miR-3619-3p gene can be obtained by a method described in Witten D et al., 2010, BMC Biol, Vol. 8, p. 58. Also, “hsa-mir-3619” (miRBase Accession No. MI0016009, SEQ ID NO: 476) having a hairpin-like structure is known as a precursor of “hsa-miR-3619-3p”.

The term “hsa-miR-671-5p gene” or “hsa-miR-671-5p” used herein includes the hsa-miR-671-5p gene (miRBase Accession No. MIMAT0003880) described in SEQ ID NO: 201, a homolog or an ortholog of a different organism species, and the like. The hsa-miR-671-5p gene can be obtained by a method described in Berezikov E et al., 2006, Genome Res, Vol. 16, p. 1289-1298. Also, “hsa-mir-671” (miRBase Accession No. MI0003760, SEQ ID NO: 477) having a hairpin-like structure is known as a precursor of “hsa-miR-671-5p”.

The term “hsa-miR-4667-5p gene” or “hsa-miR-4667-5p” used herein includes the hsa-miR-4667-5p gene (miRBase Accession No. MIMAT0019743) described in SEQ ID NO: 202, a homolog or an ortholog of a different organism species, and the like. The hsa-miR-4667-5p gene can be obtained by a method described in Persson H et al., 2011, Cancer Res, Vol. 71, p. 78-86. Also, “hsa-mir-4667” (miRBase Accession No. MI0017297, SEQ ID NO: 478) having a hairpin-like structure is known as a precursor of “hsa-miR-4667-5p”.

The term “hsa-miR-4430 gene” or “hsa-miR-4430” used herein includes the hsa-miR-4430 gene (miRBase Accession No. MIMAT0018945) described in SEQ ID NO: 203, a homolog or an ortholog of a different organism species, and the like. The hsa-miR-4430 gene can be obtained by a method described in Jima D D et al., 2010, Blood, Vol. 116, e118-e127. Also, “hsa-mir-4430” (miRBase Accession No. MI0016769, SEQ ID NO: 479) having a hairpin-like structure is known as a precursor of “hsa-miR-4430”.

The term “hsa-miR-3195 gene” or “hsa-miR-3195” used herein includes the hsa-miR-3195 gene (miRBase Accession No. MIMAT0015079) described in SEQ ID NO: 204, a homolog or an ortholog of a different organism species, and the like. The hsa-miR-3195 gene can be obtained by a method described in Stark M S et al., 2010, PLoS One, Vol. 5, e9685. Also, “hsa-mir-3195” (miRBase Accession No. MI0014240, SEQ ID NO: 480) having a hairpin-like structure is known as a precursor of “hsa-miR-3195”.

The term “hsa-miR-3679-5p gene” or “hsa-miR-3679-5p” used herein includes the hsa-miR-3679-5p gene (miRBase Accession No. MIMAT0018104) described in SEQ ID NO: 205, a homolog or an ortholog of a different organism species, and the like. The hsa-miR-3679-5p gene can be obtained by a method described in Creighton C J et al., 2010, PLoS One, Vol. 5, e9637. Also, “hsa-mir-3679” (miRBase Accession No. MI0016080, SEQ ID NO: 358) having a hairpin-like structure is known as a precursor of “hsa-miR-3679-5p”.

The term “hsa-miR-6076 gene” or “hsa-miR-6076” used herein includes the hsa-miR-6076 gene (miRBase Accession No. MIMAT0023701) described in SEQ ID NO: 206, a homolog or an ortholog of a different organism species, and the like. The hsa-miR-6076 gene can be obtained by a method described in Voellenkle C et al., 2012, RNA, Vol. 18, p. 472-484. Also, “hsa-mir-6076” (miRBase Accession No. MI0020353, SEQ ID NO: 481) having a hairpin-like structure is known as a precursor of “hsa-miR-6076”.

The term “hsa-miR-6515-5p gene” or “hsa-miR-6515-5p” used herein includes the hsa-miR-6515-5p gene (miRBase Accession No. MIMAT0025486) described in SEQ ID NO: 207, a homolog or an ortholog of a different organism species, and the like. The hsa-miR-6515-5p gene can be obtained by a method described in Joyce C E et al., 2011, Hum Mol Genet, Vol. 20, p. 4025-4040. Also, “hsa-mir-6515” (miRBase Accession No. MI0022227, SEQ ID NO: 340) having a hairpin-like structure is known as a precursor of “hsa-miR-6515-5p”.

The term “hsa-miR-6820-5p gene” or “hsa-miR-6820-5p” used herein includes the hsa-miR-6820-5p gene (miRBase Accession No. MIMAT0027540) described in SEQ ID NO: 208, a homolog or an ortholog of a different organism species, and the like. The hsa-miR-6820-5p gene can be obtained by a method described in Ladewig E et al., 2012, Genome Res, Vol. 22, p. 1634-1645. Also, “hsa-mir-6820” (miRBase Accession No. MI0022665, SEQ ID NO: 482) having a hairpin-like structure is known as a precursor of “hsa-miR-6820-5p”.

The term “hsa-miR-4634 gene” or “hsa-miR-4634” used herein includes the hsa-miR-4634 gene (miRBase Accession No. MIMAT0019691) described in SEQ ID NO: 209, a homolog or an ortholog of a different organism species, and the like. The hsa-miR-4634 gene can be obtained by a method described in Persson H et al., 2011, Cancer Res, Vol. 71, p. 78-86. Also, “hsa-mir-4634” (miRBase Accession No. MI0017261, SEQ ID NO: 483) having a hairpin-like structure is known as a precursor of “hsa-miR-4634”.

The term “hsa-miR-187-5p gene” or “hsa-miR-187-5p” used herein includes the hsa-miR-187-5p gene (miRBase Accession No. MIMAT0004561) described in SEQ ID NO: 210, a homolog or an ortholog of a different organism species, and the like. The hsa-miR-187-5p gene can be obtained by a method described in Lim L P et al., 2003, Science, Vol. 299, p. 1540. Also, “hsa-mir-187” (miRBase Accession No. MI0000274, SEQ ID NO: 484) having a hairpin-like structure is known as a precursor of “hsa-miR-187-5p”.

The term “hsa-miR-6763-5p gene” or “hsa-miR-6763-5p” used herein includes the hsa-miR-6763-5p gene (miRBase Accession No. MIMAT0027426) described in SEQ ID NO: 211, a homolog or an ortholog of a different organism species, and the like. The hsa-miR-6763-5p gene can be obtained by a method described in Ladewig E et al., 2012, Genome Res, Vol. 22, p. 1634-1645. Also, “hsa-mir-6763” (miRBase Accession No. MI0022608, SEQ ID NO: 434) having a hairpin-like structure is known as a precursor of “hsa-miR-6763-5p”.

The term “hsa-miR-1908-3p gene” or “hsa-miR-1908-3p” used herein includes the hsa-miR-1908-3p gene (miRBase Accession No. MIMAT0026916) described in SEQ ID NO: 212, a homolog or an ortholog of a different organism species, and the like. The hsa-miR-1908-3p gene can be obtained by a method described in Bar M et al., 2008, Stem Cells, Vol. 26, p. 2496-2505. Also, “hsa-mir-1908” (miRBase Accession No. MI0008329, SEQ ID NO: 393) having a hairpin-like structure is known as a precursor of “hsa-miR-1908-3p”.

The term “hsa-miR-1181 gene” or “hsa-miR-1181” used herein includes the hsa-miR-1181 gene (miRBase Accession No. MIMAT0005826) described in SEQ ID NO: 213, a homolog or an ortholog of a different organism species, and the like. The hsa-miR-1181 gene can be obtained by a method described in Subramanian S et al., 2008, Oncogene, Vol. 27, p. 2015-2026. Also, “hsa-mir-1181” (miRBase Accession No. MI0006274, SEQ ID NO: 485) having a hairpin-like structure is known as a precursor of “hsa-miR-1181”.

The term “hsa-miR-6782-5p gene” or “hsa-miR-6782-5p” used herein includes the hsa-miR-6782-5p gene (miRBase Accession No. MIMAT0027464) described in SEQ ID NO: 214, a homolog or an ortholog of a different organism species, and the like. The hsa-miR-6782-5p gene can be obtained by a method described in Ladewig E et al., 2012, Genome Res, Vol. 22, p. 1634-1645. Also, “hsa-mir-6782” (miRBase Accession No. MI0022627, SEQ ID NO: 486) having a hairpin-like structure is known as a precursor of “hsa-miR-6782-5p”.

The term “hsa-miR-5010-5p gene” or “hsa-miR-5010-5p” used herein includes the hsa-miR-5010-5p gene (miRBase Accession No. MIMAT0021043) described in SEQ ID NO: 215, a homolog or an ortholog of a different organism species, and the like. The hsa-miR-5010-5p gene can be obtained by a method described in Hansen T B et al., 2011, RNA Biol, Vol. 8, p. 378-383. Also, “hsa-mir-5010” (miRBase Accession No. MI0017878, SEQ ID NO: 487) having a hairpin-like structure is known as a precursor of “hsa-miR-5010-5p”.

The term “hsa-miR-6870-5p gene” or “hsa-miR-6870-5p” used herein includes the hsa-miR-6870-5p gene (miRBase Accession No. MIMAT0027640) described in SEQ ID NO: 216, a homolog or an ortholog of a different organism species, and the like. The hsa-miR-6870-5p gene can be obtained by a method described in Ladewig E et al., 2012, Genome Res, Vol. 22, p. 1634-1645. Also, “hsa-mir-6870” (miRBase Accession No. MI0022717, SEQ ID NO: 488) having a hairpin-like structure is known as a precursor of “hsa-miR-6870-5p”.

The term “hsa-miR-6124 gene” or “hsa-miR-6124” used herein includes the hsa-miR-6124 gene (miRBase Accession No. MIMAT0024597) described in SEQ ID NO: 217, a homolog or an ortholog of a different organism species, and the like. The hsa-miR-6124 gene can be obtained by a method described in Smith J L et al., 2012, J Virol, Vol. 86, p. 5278-5287. Also, “hsa-mir-6124” (miRBase Accession No. MI0021258, SEQ ID NO: 489) having a hairpin-like structure is known as a precursor of “hsa-miR-6124”.

The term “hsa-miR-1249-5p gene” or “hsa-miR-1249-5p” used herein includes the hsa-miR-1249-5p gene (miRBase Accession No. MIMAT0032029) described in SEQ ID NO: 218, a homolog or an ortholog of a different organism species, and the like. The hsa-miR-1249-5p gene can be obtained by a method described in Morin R D et al., 2008, Genome Res, Vol. 18, p. 610-621. Also, “hsa-mir-1249” (miRBase Accession No. MI0006384, SEQ ID NO: 321) having a hairpin-like structure is known as a precursor of “hsa-miR-1249-5p”.

The term “hsa-miR-6511b-5p gene” or “hsa-miR-6511b-5p” used herein includes the hsa-miR-6511b-5p gene (miRBase Accession No. MIMAT0025847) described in SEQ ID NO: 219, a homolog or an ortholog of a different organism species, and the like. The hsa-miR-6511b-5p gene can be obtained by a method described in Li Y et al., 2012, Gene, Vol. 497, p. 330-335. Also, “hsa-mir-6511b-1 and hsa-mir-6511b-2” (miRBase Accession Nos. MI0022552 and MI0023431, SEQ ID NOs: 490 and 491) having a hairpin-like structure are known as precursors of “hsa-miR-6511b-5p”.

The term “hsa-miR-1254 gene” or “hsa-miR-1254” used herein includes the hsa-miR-1254 gene (miRBase Accession No. MIMAT0005905) described in SEQ ID NO: 220, a homolog or an ortholog of a different organism species, and the like. The hsa-miR-1254 gene can be obtained by a method described in Morin R D et al., 2008, Genome Res, Vol. 18, p. 610-621. Also, “hsa-mir-1254-1 and hsa-mir-1254-2” (miRBase Accession Nos. MI0006388 and MI0016747, SEQ ID NOs: 492 and 493) having a hairpin-like structure are known as precursors of “hsa-miR-1254”.

The term “hsa-miR-4727-3p gene” or “hsa-miR-4727-3p” used herein includes the hsa-miR-4727-3p gene (miRBase Accession No. MIMAT0019848) described in SEQ ID NO: 221, a homolog or an ortholog of a different organism species, and the like. The hsa-miR-4727-3p gene can be obtained by a method described in Persson H et al., 2011, Cancer Res, Vol. 71, p. 78-86. Also, “hsa-mir-4727” (miRBase Accession No. MI0017364, SEQ ID NO: 494) having a hairpin-like structure is known as a precursor of “hsa-miR-4727-3p”.

The term “hsa-miR-4259 gene” or “hsa-miR-4259” used herein includes the hsa-miR-4259 gene (miRBase Accession No. MIMAT0016880) described in SEQ ID NO: 222, a homolog or an ortholog of a different organism species, and the like. The hsa-miR-4259 gene can be obtained by a method described in Goff L A et al., 2009, PLoS One, Vol. 4, e7192. Also, “hsa-mir-4259” (miRBase Accession No. MI0015858, SEQ ID NO: 495) having a hairpin-like structure is known as a precursor of “hsa-miR-4259”.

The term “hsa-miR-4771 gene” or “hsa-miR-4771” used herein includes the hsa-miR-4771 gene (miRBase Accession No. MIMAT0019925) described in SEQ ID NO: 223, a homolog or an ortholog of a different organism species, and the like. The hsa-miR-4771 gene can be obtained by a method described in Persson H et al., 2011, Cancer Res, Vol. 71, p. 78-86. Also, “hsa-mir-4771-1 and hsa-mir-4771-2” (miRBase Accession Nos. MI0017412 and MI0017413, SEQ ID NOs: 496 and 497) having a hairpin-like structure are known as precursors of “hsa-miR-4771”.

The term “hsa-miR-3622a-5p gene” or “hsa-miR-3622a-5p” used herein includes the hsa-miR-3622a-5p gene (miRBase Accession No. MIMAT0018003) described in SEQ ID NO: 224, a homolog or an ortholog of a different organism species, and the like. The hsa-miR-3622a-5p gene can be obtained by a method described in Witten D et al., 2010, BMC Biol, Vol. 8, p. 58. Also, “hsa-mir-3622a” (miRBase Accession No. MI0016013, SEQ ID NO: 498) having a hairpin-like structure is known as a precursor of “hsa-miR-3622a-5p”.

The term “hsa-miR-4480 gene” or “hsa-miR-4480” used herein includes the hsa-miR-4480 gene (miRBase Accession No. MIMAT0019014) described in SEQ ID NO: 225, a homolog or an ortholog of a different organism species, and the like. The hsa-miR-4480 gene can be obtained by a method described in Jima D D et al., 2010, Blood, Vol. 116, e118-e127. Also, “hsa-mir-4480” (miRBase Accession No. MI0016841, SEQ ID NO: 499) having a hairpin-like structure is known as a precursor of “hsa-miR-4480”.

The term “hsa-miR-4740-5p gene” or “hsa-miR-4740-5p” used herein includes the hsa-miR-4740-5p gene (miRBase Accession No. MIMAT0019869) described in SEQ ID NO: 226, a homolog or an ortholog of a different organism species, and the like. The hsa-miR-4740-5p gene can be obtained by a method described in Persson H et al., 2011, Cancer Res, Vol. 71, p. 78-86. Also, “hsa-mir-4740” (miRBase Accession No. MI0017378, SEQ ID NO: 500) having a hairpin-like structure is known as a precursor of “hsa-miR-4740-5p”.

The term “hsa-miR-6777-5p gene” or “hsa-miR-6777-5p” used herein includes the hsa-miR-6777-5p gene (miRBase Accession No. MIMAT0027454) described in SEQ ID NO: 227, a homolog or an ortholog of a different organism species, and the like. The hsa-miR-6777-5p gene can be obtained by a method described in Ladewig E et al., 2012, Genome Res, Vol. 22, p. 1634-1645. Also, “hsa-mir-6777” (miRBase Accession No. MI0022622, SEQ ID NO: 501) having a hairpin-like structure is known as a precursor of “hsa-miR-6777-5p”.

The term “hsa-miR-6794-5p gene” or “hsa-miR-6794-5p” used herein includes the hsa-miR-6794-5p gene (miRBase Accession No. MIMAT0027488) described in SEQ ID NO: 228, a homolog or an ortholog of a different organism species, and the like. The hsa-miR-6794-5p gene can be obtained by a method described in Ladewig E et al., 2012, Genome Res, Vol. 22, p. 1634-1645. Also, “hsa-mir-6794” (miRBase Accession No. MI0022639, SEQ ID NO: 502) having a hairpin-like structure is known as a precursor of “hsa-miR-6794-5p”.

The term “hsa-miR-4687-3p gene” or “hsa-miR-4687-3p” used herein includes the hsa-miR-4687-3p gene (miRBase Accession No. MIMAT0019775) described in SEQ ID NO: 229, a homolog or an ortholog of a different organism species, and the like. The hsa-miR-4687-3p gene can be obtained by a method described in Persson H et al., 2011, Cancer Res, Vol. 71, p. 78-86. Also, “hsa-mir-4687” (miRBase Accession No. MI0017319, SEQ ID NO: 341) having a hairpin-like structure is known as a precursor of “hsa-miR-4687-3p”.

The term “hsa-miR-6743-5p gene” or “hsa-miR-6743-5p” used herein includes the hsa-miR-6743-5p gene (miRBase Accession No. MIMAT0027387) described in SEQ ID NO: 230, a homolog or an ortholog of a different organism species, and the like. The hsa-miR-6743-5p gene can be obtained by a method described in Ladewig E et al., 2012, Genome Res, Vol. 22, p. 1634-1645. Also, “hsa-mir-6743” (miRBase Accession No. MI0022588, SEQ ID NO: 503) having a hairpin-like structure is known as a precursor of “hsa-miR-6743-5p”.

The term “hsa-miR-6771-5p gene” or “hsa-miR-6771-5p” used herein includes the hsa-miR-6771-5p gene (miRBase Accession No. MIMAT0027442) described in SEQ ID NO: 231, a homolog or an ortholog of a different organism species, and the like. The hsa-miR-6771-5p gene can be obtained by a method described in Ladewig E et al., 2012, Genome Res, Vol. 22, p. 1634-1645. Also, “hsa-mir-6771” (miRBase Accession No. MI0022616, SEQ ID NO: 504) having a hairpin-like structure is known as a precursor of “hsa-miR-6771-5p”.

The term “hsa-miR-3141 gene” or “hsa-miR-3141” used herein includes the hsa-miR-3141 gene (miRBase Accession No. MIMAT0015010) described in SEQ ID NO: 232, a homolog or an ortholog of a different organism species, and the like. The hsa-miR-3141 gene can be obtained by a method described in Stark M S et al., 2010, PLoS One, Vol. 5, e9685. Also, “hsa-mir-3141” (miRBase Accession No. MI0014165, SEQ ID NO: 505) having a hairpin-like structure is known as a precursor of “hsa-miR-3141”.

The term “hsa-miR-3162-5p gene” or “hsa-miR-3162-5p” used herein includes the hsa-miR-3162-5p gene (miRBase Accession No. MIMAT0015036) described in SEQ ID NO: 233, a homolog or an ortholog of a different organism species, and the like. The hsa-miR-3162-5p gene can be obtained by a method described in Stark M S et al., 2010, PLoS One, Vol. 5, e9685. Also, “hsa-mir-3162” (miRBase Accession No. MI0014192, SEQ ID NO: 506) having a hairpin-like structure is known as a precursor of “hsa-miR-3162-5p”.

The term “hsa-miR-4271 gene” or “hsa-miR-4271” used herein includes the hsa-miR-4271 gene (miRBase Accession No. MIMAT0016901) described in SEQ ID NO: 234, a homolog or an ortholog of a different organism species, and the like. The hsa-miR-4271 gene can be obtained by a method described in Goff L A et al., 2009, PLoS One, Vol. 4, e7192. Also, “hsa-mir-4271” (miRBase Accession No. MI0015879, SEQ ID NO: 507) having a hairpin-like structure is known as a precursor of “hsa-miR-4271”.

The term “hsa-miR-1227-5p gene” or “hsa-miR-1227-5p” used herein includes the hsa-miR-1227-5p gene (miRBase Accession No. MIMAT0022941) described in SEQ ID NO: 235, a homolog or an ortholog of a different organism species, and the like. The hsa-miR-1227-5p gene can be obtained by a method described in Berezikov E et al., 2007, Mol Cell, Vol. 28, p. 328-336. Also, “hsa-mir-1227” (miRBase Accession No. MI0006316, SEQ ID NO: 508) having a hairpin-like structure is known as a precursor of “hsa-miR-1227-5p”.

The term “hsa-miR-4257 gene” or “hsa-miR-4257” used herein includes the hsa-miR-4257 gene (miRBase Accession No. MIMAT0016878) described in SEQ ID NO: 236, a homolog or an ortholog of a different organism species, and the like. The hsa-miR-4257 gene can be obtained by a method described in Goff L A et al., 2009, PLoS One, Vol. 4, e7192. Also, “hsa-mir-4257” (miRBase Accession No. MI0015856, SEQ ID NO: 509) having a hairpin-like structure is known as a precursor of “hsa-miR-4257”.

The term “hsa-miR-4270 gene” or “hsa-miR-4270” used herein includes the hsa-miR-4270 gene (miRBase Accession No. MIMAT0016900) described in SEQ ID NO: 237, a homolog or an ortholog of a different organism species, and the like. The hsa-miR-4270 gene can be obtained by a method described in Goff L A et al., 2009, PLoS One, Vol. 4, e7192. Also, “hsa-mir-4270” (miRBase Accession No. MI0015878, SEQ ID NO: 510) having a hairpin-like structure is known as a precursor of “hsa-miR-4270”.

The term “hsa-miR-4516 gene” or “hsa-miR-4516” used herein includes the hsa-miR-4516 gene (miRBase Accession No. MIMAT0019053) described in SEQ ID NO: 238, a homolog or an ortholog of a different organism species, and the like. The hsa-miR-4516 gene can be obtained by a method described in Jima D D et al., 2010, Blood, Vol. 116, e118-e127. Also, “hsa-mir-4516” (miRBase Accession No. MI0016882, SEQ ID NO: 511) having a hairpin-like structure is known as a precursor of “hsa-miR-4516”.

The term “hsa-miR-4651 gene” or “hsa-miR-4651” used herein includes the hsa-miR-4651 gene (miRBase Accession No. MIMAT0019715) described in SEQ ID NO: 239, a homolog or an ortholog of a different organism species, and the like. The hsa-miR-4651 gene can be obtained by a method described in Persson H et al., 2011, Cancer Res, Vol. 71, p. 78-86. Also, “hsa-mir-4651” (miRBase Accession No. MI0017279, SEQ ID NO: 512) having a hairpin-like structure is known as a precursor of “hsa-miR-4651”.

The term “hsa-miR-4725-3p gene” or “hsa-miR-4725-3p” used herein includes the hsa-miR-4725-3p gene (miRBase Accession No. MIMAT0019844) described in SEQ ID NO: 240, a homolog or an ortholog of a different organism species, and the like. The hsa-miR-4725-3p gene can be obtained by a method described in Persson H et al., 2011, Cancer Res, Vol. 71, p. 78-86. Also, “hsa-mir-4725” (miRBase Accession No. MI0017362, SEQ ID NO: 513) having a hairpin-like structure is known as a precursor of “hsa-miR-4725-3p”.

The term “hsa-miR-6125 gene” or “hsa-miR-6125” used herein includes the hsa-miR-6125 gene (miRBase Accession No. MIMAT0024598) described in SEQ ID NO: 241, a homolog or an ortholog of a different organism species, and the like. The hsa-miR-6125 gene can be obtained by a method described in Smith J L et al., 2012, J Virol, Vol. 86, p. 5278-5287. Also, “hsa-mir-6125” (miRBase Accession No. MI0021259, SEQ ID NO: 514) having a hairpin-like structure is known as a precursor of “hsa-miR-6125”.

The term “hsa-miR-6732-5p gene” or “hsa-miR-6732-5p” used herein includes the hsa-miR-6732-5p gene (miRBase Accession No. MIMAT0027365) described in SEQ ID NO: 242, a homolog or an ortholog of a different organism species, and the like. The hsa-miR-6732-5p gene can be obtained by a method described in Ladewig E et al., 2012, Genome Res, Vol. 22, p. 1634-1645. Also, “hsa-mir-6732” (miRBase Accession No. MI0022577, SEQ ID NO: 515) having a hairpin-like structure is known as a precursor of “hsa-miR-6732-5p”.

The term “hsa-miR-6791-5p gene” or “hsa-miR-6791-5p” used herein includes the hsa-miR-6791-5p gene (miRBase Accession No. MIMAT0027482) described in SEQ ID NO: 243, a homolog or an ortholog of a different organism species, and the like. The hsa-miR-6791-5p gene can be obtained by a method described in Ladewig E et al., 2012, Genome Res, Vol. 22, p. 1634-1645. Also, “hsa-mir-6791” (miRBase Accession No. MI0022636, SEQ ID NO: 516) having a hairpin-like structure is known as a precursor of “hsa-miR-6791-5p”.

The term “hsa-miR-6819-5p gene” or “hsa-miR-6819-5p” used herein includes the hsa-miR-6819-5p gene (miRBase Accession No. MIMAT0027538) described in SEQ ID NO: 244, a homolog or an ortholog of a different organism species, and the like. The hsa-miR-6819-5p gene can be obtained by a method described in Ladewig E et al., 2012, Genome Res, Vol. 22, p. 1634-1645. Also, “hsa-mir-6819” (miRBase Accession No. MI0022664, SEQ ID NO: 517) having a hairpin-like structure is known as a precursor of “hsa-miR-6819-5p”.

The term “hsa-miR-6891-5p gene” or “hsa-miR-6891-5p” used herein includes the hsa-miR-6891-5p gene (miRBase Accession No. MIMAT0027682) described in SEQ ID NO: 245, a homolog or an ortholog of a different organism species, and the like. The hsa-miR-6891-5p gene can be obtained by a method described in Ladewig E et al., 2012, Genome Res, Vol. 22, p. 1634-1645. Also, “hsa-mir-6891” (miRBase Accession No. MI0022738, SEQ ID NO: 518) having a hairpin-like structure is known as a precursor of “hsa-miR-6891-5p”.

The term “hsa-miR-7108-5p gene” or “hsa-miR-7108-5p” used herein includes the hsa-miR-7108-5p gene (miRBase Accession No. MIMAT0028113) described in SEQ ID NO: 246, a homolog or an ortholog of a different organism species, and the like. The hsa-miR-7108-5p gene can be obtained by a method described in Ladewig E et al., 2012, Genome Res, Vol. 22, p. 1634-1645. Also, “hsa-mir-7108” (miRBase Accession No. MI0022959, SEQ ID NO: 448) having a hairpin-like structure is known as a precursor of “hsa-miR-7108-5p”.

The term “hsa-miR-7109-5p gene” or “hsa-miR-7109-5p” used herein includes the hsa-miR-7109-5p gene (miRBase Accession No. MIMAT0028115) described in SEQ ID NO: 247, a homolog or an ortholog of a different organism species, and the like. The hsa-miR-7109-5p gene can be obtained by a method described in Ladewig E et al., 2012, Genome Res, Vol. 22, p. 1634-1645. Also, “hsa-mir-7109” (miRBase Accession No. MI0022960, SEQ ID NO: 519) having a hairpin-like structure is known as a precursor of “hsa-miR-7109-5p”.

The term “hsa-miR-320a gene” or “hsa-miR-320a” used herein includes the hsa-miR-320a gene (miRBase Accession No. MIMAT0000510) described in SEQ ID NO: 248, a homolog or an ortholog of a different organism species, and the like. The hsa-miR-320a gene can be obtained by a method described in Michael M Z et al., 2003, Mol Cancer Res, Vol. 1, p. 882-891. Also, “hsa-mir-320a” (miRBase Accession No. MI0000542, SEQ ID NO: 520) having a hairpin-like structure is known as a precursor of “hsa-miR-320a”.

The term “hsa-miR-663a gene” or “hsa-miR-663a” used herein includes the hsa-miR-663a gene (miRBase Accession No. MIMAT0003326) described in SEQ ID NO: 249, a homolog or an ortholog of a different organism species, and the like. The hsa-miR-663a gene can be obtained by a method described in Cummins J M et al., 2006, Proc Natl Acad Sci USA, Vol. 103, p. 3687-3692. Also, “hsa-mir-663a” (miRBase Accession No. MI0003672, SEQ ID NO: 521) having a hairpin-like structure is known as a precursor of “hsa-miR-663a”.

The term “hsa-miR-328-5p gene” or “hsa-miR-328-5p” used herein includes the hsa-miR-328-5p gene (miRBase Accession No. MIMAT0026486) described in SEQ ID NO: 250, a homolog or an ortholog of a different organism species, and the like. The hsa-miR-328-5p gene can be obtained by a method described in Kim J et al., 2004, Proc Natl Acad Sci USA, Vol. 101, p. 360-365. Also, “hsa-mir-328” (miRBase Accession No. MI0000804, SEQ ID NO: 522) having a hairpin-like structure is known as a precursor of “hsa-miR-328-5p”.

The term “hsa-miR-642b-3p gene” or “hsa-miR-642b-3p” used herein includes the hsa-miR-642b-3p gene (miRBase Accession No. MIMAT0018444) described in SEQ ID NO: 251, a homolog or an ortholog of a different organism species, and the like. The hsa-miR-642b-3p gene can be obtained by a method described in Witten D et al., 2010, BMC Biol, Vol. 8, p. 58. Also, “hsa-mir-642b” (miRBase Accession No. MI0016685, SEQ ID NO: 523) having a hairpin-like structure is known as a precursor of “hsa-miR-642b-3p”.

The term “hsa-miR-128-2-5p gene” or “hsa-miR-128-2-5p” used herein includes the hsa-miR-128-2-5p gene (miRBase Accession No. MIMAT0031095) described in SEQ ID NO: 252, a homolog or an ortholog of a different organism species, and the like. The hsa-miR-128-2-5p gene can be obtained by a method described in Lagos-Quintana M et al., 2002, Curr Biol, Vol. 12, p. 735-739. Also, “hsa-mir-128-2” (miRBase Accession No. MI0000727, SEQ ID NO: 524) having a hairpin-like structure is known as a precursor of “hsa-miR-128-2-5p”.

The term “hsa-miR-125a-3p gene” or “hsa-miR-125a-3p” used herein includes the hsa-miR-125a-3p gene (miRBase Accession No. MIMAT0004602) described in SEQ ID NO: 253, a homolog or an ortholog of a different organism species, and the like. The hsa-miR-125a-3p gene can be obtained by a method described in Lagos-Quintana M et al., 2002, Curr Biol, Vol. 12, p. 735-739. Also, “hsa-mir-125a” (miRBase Accession No. MI0000469, SEQ ID NO: 525) having a hairpin-like structure is known as a precursor of “hsa-miR-125a-3p”.

The term “hsa-miR-191-5p gene” or “hsa-miR-191-5p” used herein includes the hsa-miR-191-5p gene (miRBase Accession No. MIMAT0000440) described in SEQ ID NO: 254, a homolog or an ortholog of a different organism species, and the like. The hsa-miR-191-5p gene can be obtained by a method described in Lagos-Quintana M et al., 2003, RNA, Vol. 9, p. 175-179. Also, “hsa-mir-191” (miRBase Accession No. MI0000465, SEQ ID NO: 526) having a hairpin-like structure is known as a precursor of “hsa-miR-191-5p”.

The term “hsa-miR-92b-5p gene” or “hsa-miR-92b-5p” used herein includes the hsa-miR-92b-5p gene (miRBase Accession No. MIMAT0004792) described in SEQ ID NO: 255, a homolog or an ortholog of a different organism species, and the like. The hsa-miR-92b-5p gene can be obtained by a method described in Cummins J M et al., 2006, Proc Natl Acad Sci USA, Vol. 103, p. 3687-3692. Also, “hsa-mir-92b” (miRBase Accession No. MI0003560, SEQ ID NO: 527) having a hairpin-like structure is known as a precursor of “hsa-miR-92b-5p”.

The term “hsa-miR-296-5p gene” or “hsa-miR-296-5p” used herein includes the hsa-miR-296-5p gene (miRBase Accession No. MIMAT0000690) described in SEQ ID NO: 256, a homolog or an ortholog of a different organism species, and the like. The hsa-miR-296-5p gene can be obtained by a method described in Houbaviy H B et al., 2003, Dev Cell, Vol. 5, p. 351-358. Also, “hsa-mir-296” (miRBase Accession No. MI0000747, SEQ ID NO: 336) having a hairpin-like structure is known as a precursor of “hsa-miR-296-5p”.

The term “hsa-miR-1246 gene” or “hsa-miR-1246” used herein includes the hsa-miR-1246 gene (miRBase Accession No. MIMAT0005898) described in SEQ ID NO: 257, a homolog or an ortholog of a different organism species, and the like. The hsa-miR-1246 gene can be obtained by a method described in Morin R D et al., 2008, Genome Res, Vol. 18, p. 610-621. Also, “hsa-mir-1246” (miRBase Accession No. MI0006381, SEQ ID NO: 528) having a hairpin-like structure is known as a precursor of “hsa-miR-1246”.

The term “hsa-miR-92a-2-5p gene” or “hsa-miR-92a-2-5p” used herein includes the hsa-miR-92a-2-5p gene (miRBase Accession No. MIMAT0004508) described in SEQ ID NO: 258, a homolog or an ortholog of a different organism species, and the like. The hsa-miR-92a-2-5p gene can be obtained by a method described in Mourelatos Z et al., 2002, Genes Dev, Vol. 16, p. 720-728. Also, “hsa-mir-92a-2” (miRBase Accession No. MI0000094, SEQ ID NO: 529) having a hairpin-like structure is known as a precursor of “hsa-miR-92a-2-5p”.

The term “hsa-miR-128-1-5p gene” or “hsa-miR-128-1-5p” used herein includes the hsa-miR-128-1-5p gene (miRBase Accession No. MIMAT0026477) described in SEQ ID NO: 259, a homolog or an ortholog of a different organism species, and the like. The hsa-miR-128-1-5p gene can be obtained by a method described in Lagos-Quintana M et al., 2002, Curr Biol, Vol. 12, p. 735-739. Also, “hsa-mir-128-1” (miRBase Accession No. MI0000447, SEQ ID NO: 530) having a hairpin-like structure is known as a precursor of “hsa-miR-128-1-5p”.

The term “hsa-miR-1290 gene” or “hsa-miR-1290” used herein includes the hsa-miR-1290 gene (miRBase Accession No. MIMAT0005880) described in SEQ ID NO: 260, a homolog or an ortholog of a different organism species, and the like. The hsa-miR-1290 gene can be obtained by a method described in Morin R D et al., 2008, Genome Res, Vol. 18, p. 610-621. Also, “hsa-mir-1290” (miRBase Accession No. MI0006352, SEQ ID NO: 531) having a hairpin-like structure is known as a precursor of “hsa-miR-1290”.

The term “hsa-miR-211-3p gene” or “hsa-miR-211-3p” used herein includes the hsa-miR-211-3p gene (miRBase Accession No. MIMAT0022694) described in SEQ ID NO: 261, a homolog or an ortholog of a different organism species, and the like. The hsa-miR-211-3p gene can be obtained by a method described in Lim L P et al., 2003, Science, Vol. 299, p. 1540. Also, “hsa-mir-211” (miRBase Accession No. MI0000287, SEQ ID NO: 532) having a hairpin-like structure is known as a precursor of “hsa-miR-211-3p”.

The term “hsa-miR-744-5p gene” or “hsa-miR-744-5p” used herein includes the hsa-miR-744-5p gene (miRBase Accession No. MIMAT0004945) described in SEQ ID NO: 262, a homolog or an ortholog of a different organism species, and the like. The hsa-miR-744-5p gene can be obtained by a method described in Berezikov E et al., 2006, Genome Res, Vol. 16, p. 1289-1298. Also, “hsa-mir-744” (miRBase Accession No. MI0005559, SEQ ID NO: 533) having a hairpin-like structure is known as a precursor of “hsa-miR-744-5p”.

The term “hsa-miR-135a-3p gene” or “hsa-miR-135a-3p” used herein includes the hsa-miR-135a-3p gene (miRBase Accession No. MIMAT0004595) described in SEQ ID NO: 263, a homolog or an ortholog of a different organism species, and the like. The hsa-miR-135a-3p gene can be obtained by a method described in Lagos-Quintana M et al., 2002, Curr Biol, Vol. 12, p. 735-739. Also, “hsa-mir-135a-1” (miRBase Accession No. MI0000452, SEQ ID NO: 534) having a hairpin-like structure is known as a precursor of “hsa-miR-135a-3p”.

The term “hsa-miR-451a gene” or “hsa-miR-451a” used herein includes the hsa-miR-451a gene (miRBase Accession No. MIMAT0001631) described in SEQ ID NO: 264, a homolog or an ortholog of a different organism species, and the like. The hsa-miR-451a gene can be obtained by a method described in Altuvia Y et al., 2005, Nucleic Acids Res, Vol. 33, p. 2697-2706. Also, “hsa-mir-451a” (miRBase Accession No. MI0001729, SEQ ID NO: 535) having a hairpin-like structure is known as a precursor of “hsa-miR-451a”.

The term “hsa-miR-625-3p gene” or “hsa-miR-625-3p” used herein includes the hsa-miR-625-3p gene (miRBase Accession No. MIMAT0004808) described in SEQ ID NO: 265, a homolog or an ortholog of a different organism species, and the like. The hsa-miR-625-3p gene can be obtained by a method described in Cummins J M et al., 2006, Proc Natl Acad Sci USA, Vol. 103, p. 3687-3692. Also, “hsa-mir-625” (miRBase Accession No. MI0003639, SEQ ID NO: 536) having a hairpin-like structure is known as a precursor of “hsa-miR-625-3p”.

The term “hsa-miR-92a-3p gene” or “hsa-miR-92a-3p” used herein includes the hsa-miR-92a-3p gene (miRBase Accession No. MIMAT0000092) described in SEQ ID NO: 266, a homolog or an ortholog of a different organism species, and the like. The hsa-miR-92a-3p gene can be obtained by a method described in Mourelatos Z et al., 2002, Genes Dev, Vol. 16, p. 720-728. Also, “hsa-mir-92a-1 and hsa-mir-92a-2” (miRBase Accession Nos. MI0000093 and MI0000094, SEQ ID NOs: 537 and 529) having a hairpin-like structure are known as precursors of “hsa-miR-92a-3p”.

The term “hsa-miR-422a gene” or “hsa-miR-422a” used herein includes the hsa-miR-422a gene (miRBase Accession No. MIMAT0001339) described in SEQ ID NO: 267, a homolog or an ortholog of a different organism species, and the like. The hsa-miR-422a gene can be obtained by a method described in Kasashima K et al., 2004, Biochem Biophys Res Commun, Vol. 322, p. 403-410. Also, “hsa-mir-422a” (miRBase Accession No. MI0001444, SEQ ID NO: 538) having a hairpin-like structure is known as a precursor of “hsa-miR-422a”.

The term “hsa-miR-642a-3p gene” or “hsa-miR-642a-3p” used herein includes the hsa-miR-642a-3p gene (miRBase Accession No. MIMAT0020924) described in SEQ ID NO: 268, a homolog or an ortholog of a different organism species, and the like. The hsa-miR-642a-3p gene can be obtained by a method described in Cummins J M et al., 2006, Proc Natl Acad Sci USA, Vol. 103, p. 3687-3692. Also, “hsa-mir-642a” (miRBase Accession No. MI0003657, SEQ ID NO: 539) having a hairpin-like structure is known as a precursor of “hsa-miR-642a-3p”.

The term “hsa-miR-483-5p gene” or “hsa-miR-483-5p” used herein includes the hsa-miR-483-5p gene (miRBase Accession No. MIMAT0004761) described in SEQ ID NO: 269, a homolog or an ortholog of a different organism species, and the like. The hsa-miR-483-5p gene can be obtained by a method described in Fu H et al., 2005, FEBS Lett, Vol. 579, p. 3849-3854. Also, “hsa-mir-483” (miRBase Accession No. MI0002467, SEQ ID NO: 540) having a hairpin-like structure is known as a precursor of “hsa-miR-483-5p”.

The term “hsa-miR-652-5p gene” or “hsa-miR-652-5p” used herein includes the hsa-miR-652-5p gene (miRBase Accession No. MIMAT0022709) described in SEQ ID NO: 270, a homolog or an ortholog of a different organism species, and the like. The hsa-miR-652-5p gene can be obtained by a method described in Cummins J M et al., 2006, Proc Natl Acad Sci USA, Vol. 103, p. 3687-3692. Also, “hsa-mir-652” (miRBase Accession No. MI0003667, SEQ ID NO: 541) having a hairpin-like structure is known as a precursor of “hsa-miR-652-5p”.

The term “hsa-miR-24-3p gene” or “hsa-miR-24-3p” used herein includes the hsa-miR-24-3p gene (miRBase Accession No. MIMAT0000080) described in SEQ ID NO: 271, a homolog or an ortholog of a different organism species, and the like. The hsa-miR-24-3p gene can be obtained by a method described in Lagos-Quintana M et al., 2001, Science, Vol. 294, p. 853-858. Also, “hsa-mir-24-1 and hsa-mir-24-2” (miRBase Accession Nos. MI0000080 and MI0000081, SEQ ID NOs: 542 and 543) having a hairpin-like structure are known as precursors of “hsa-miR-24-3p”.

The term “hsa-miR-23b-3p gene” or “hsa-miR-23b-3p” used herein includes the hsa-miR-23b-3p gene (miRBase Accession No. MIMAT0000418) described in SEQ ID NO: 272, a homolog or an ortholog of a different organism species, and the like. The hsa-miR-23b-3p gene can be obtained by a method described in Lagos-Quintana M et al., 2002, Curr Biol, Vol. 12, p. 735-739. Also, “hsa-mir-23b” (miRBase Accession No. MI0000439, SEQ ID NO: 544) having a hairpin-like structure is known as a precursor of “hsa-miR-23b-3p”.

The term “hsa-miR-23a-3p gene” or “hsa-miR-23a-3p” used herein includes the hsa-miR-23a-3p gene (miRBase Accession No. MIMAT0000078) described in SEQ ID NO: 273, a homolog or an ortholog of a different organism species, and the like. The hsa-miR-23a-3p gene can be obtained by a method described in Lagos-Quintana M et al., 2001, Science, Vol. 294, p. 853-858. Also, “hsa-mir-23a” (miRBase Accession No. MI0000079, SEQ ID NO: 545) having a hairpin-like structure is known as a precursor of “hsa-miR-23a-3p”.

The term “hsa-miR-92b-3p gene” or “hsa-miR-92b-3p” used herein includes the hsa-miR-92b-3p gene (miRBase Accession No. MIMAT0003218) described in SEQ ID NO: 274, a homolog or an ortholog of a different organism species, and the like. The hsa-miR-92b-3p gene can be obtained by a method described in Cummins J M et al., 2006, Proc Natl Acad Sci USA, Vol. 103, p. 3687-3692. Also, “hsa-mir-92b” (miRBase Accession No. MI0003560, SEQ ID NO: 527) having a hairpin-like structure is known as a precursor of “hsa-miR-92b-3p”.

The term “hsa-miR-22-3p gene” or “hsa-miR-22-3p” used herein includes the hsa-miR-22-3p gene (miRBase Accession No. MIMAT0000077) described in SEQ ID NO: 275, a homolog or an ortholog of a different organism species, and the like. The hsa-miR-22-3p gene can be obtained by a method described in Lagos-Quintana M et al., 2001, Science, Vol. 294, p. 853-858. Also, “hsa-mir-22” (miRBase Accession No. MI0000078, SEQ ID NO: 546) having a hairpin-like structure is known as a precursor of “hsa-miR-22-3p”.

A mature miRNA may become a variant due to the sequence cleaved shorter or longer by one to several flanking nucleotides, or due to substitution of nucleotides, when cut out as the mature miRNA from its RNA precursor having a hairpin-like structure. This variant is called isomiR (Morin R D. et al., 2008, Genome Res., Vol. 18, p. 610-621). The miRBase Release 21 shows the nucleotide sequences represented by SEQ ID NOs: 1 to 275 as well as a large number of the nucleotide sequence variants and fragments represented by SEQ ID NOs: 547 to 842, called isomiRs. These variants can also be obtained as miRNAs having a nucleotide sequence represented by any of SEQ ID NOs: 1 to 275. Specifically, among the variants of polynucleotides consisting of the nucleotide sequence represented by any of SEQ ID NOs: 2, 3, 4, 8, 11, 14, 17, 18, 20, 24, 25, 29, 34, 36, 39, 40, 41, 42, 43, 44, 45, 51, 53, 55, 57, 59, 62, 63, 66, 67, 69, 70, 71, 72, 73, 75, 76, 78, 79, 84, 87, 88, 90, 94, 96, 98, 101, 102, 103, 104, 105, 110, 112, 113, 117, 119, 121, 122, 123, 124, 125, 127, 130, 136, 140, 141, 146, 148, 151, 153, 154, 159, 164, 166, 170, 175, 180, 184, 185, 189, 190, 193, 195, 196, 197, 198, 199, 201, 202, 203, 204, 205, 207, 210, 212, 213, 215, 217, 218, 219, 220, 221, 223, 224, 229, 232, 233, 238, 239, 240, 241, 248, 249, 250, 251, 252, 253, 254, 255, 256, 257, 258, 259, 260, 261, 262, 263, 264, 265, 266, 269, 270, 271, 272, 274, and 275 or a nucleotide sequence derived from the nucleotide sequence by the replacement of u with t according to the present invention, examples of the longest variants registered in miRBase Release 21 include polynucleotides represented by SEQ ID NOs: 547, 549, 551, 554, 556, 560, 563, 565, 567, 570, 572, 574, 576, 578, 580, 582, 584, 586, 588, 590, 592, 594, 596, 598, 600, 602, 604, 606, 608, 610, 612, 614, 616, 618, 620, 623, 625, 627, 629, 632, 634, 636, 638, 641, 644, 646, 649, 651, 653, 655, 657, 661, 663, 665, 668, 670, 672, 674, 676, 678, 680, 682, 685, 687, 689, 691, 694, 696, 698, 700, 702, 704, 707, 709, 711, 714, 717, 720, 722, 725, 727, 729, 731, 733, 735, 737, 739, 741, 743, 745, 747, 749, 751, 754, 756, 758, 760, 762, 764, 766, 768, 770, 772, 774, 776, 778, 780, 783, 785, 787, 789, 791, 793, 795, 797, 799, 801, 803, 805, 807, 809, 811, 813, 815, 817, 819, 821, 823, 825, 827, 830, 832, 834, 836, 839, and 841, respectively. Also, among the variants of polynucleotides consisting of a nucleotide sequence represented by any of SEQ ID NOs: 2, 3, 4, 8, 11, 14, 17, 18, 20, 24, 25, 29, 34, 36, 39, 40, 41, 42, 43, 44, 45, 51, 53, 55, 57, 59, 62, 63, 66, 67, 69, 70, 71, 72, 73, 75, 76, 78, 79, 84, 87, 88, 90, 94, 96, 98, 101, 102, 103, 104, 105, 110, 112, 113, 117, 119, 121, 122, 123, 124, 125, 127, 130, 136, 140, 141, 146, 148, 151, 153, 154, 159, 164, 166, 170, 175, 180, 184, 185, 189, 190, 193, 195, 196, 197, 198, 199, 201, 202, 203, 204, 205, 207, 210, 212, 213, 215, 217, 218, 219, 220, 221, 223, 224, 229, 232, 233, 238, 239, 240, 241, 248, 249, 250, 251, 252, 253, 254, 255, 256, 257, 258, 259, 260, 261, 262, 263, 264, 265, 266, 269, 270, 271, 272, 274, and 275 or a nucleotide sequence derived from the nucleotide sequence by the replacement of u with t according to the present invention, examples of shortest variants registered in the miRBase Release 21 include polynucleotides having sequences represented by SEQ ID NOs: 548, 550, 552, 555, 557, 561, 564, 566, 568, 571, 573, 575, 577, 579, 581, 583, 585, 587, 589, 591, 593, 595, 597, 599, 601, 603, 605, 607, 609, 611, 613, 615, 617, 619, 621, 624, 626, 628, 630, 633, 635, 637, 639, 642, 645, 647, 650, 652, 654, 656, 658, 662, 664, 666, 669, 671, 673, 675, 677, 679, 681, 683, 686, 688, 690, 692, 695, 697, 699, 701, 703, 705, 708, 710, 712, 715, 718, 721, 723, 726, 728, 730, 732, 734, 736, 738, 740, 742, 744, 746, 748, 750, 752, 755, 757, 759, 761, 763, 765, 767, 769, 771, 773, 775, 777, 779, 781, 784, 786, 788, 790, 792, 794, 796, 798, 800, 802, 804, 806, 808, 810, 812, 814, 816, 818, 820, 822, 824, 826, 828, 831, 833, 835, 837, 840, and 842, respectively. In addition to these variants and fragments, examples thereof include a large number of isomiR polynucleotides of SEQ ID NOs: 1 to 275 registered in the miRBase. Examples of the polynucleotide comprising a nucleotide sequence represented by any of SEQ ID NOs: 1 to 275 include a polynucleotide represented by any of SEQ ID NOs: 276 to 546, which are their respective precursors.

The names and miRBase Accession Nos. (registration numbers) of the genes represented by SEQ ID NOs: 1 to 842 are shown in Table 1.

As used herein, the term “capable of specifically binding” means that nucleic acids such as the nucleic acid probe or the primer used in the present invention binds to a particular target nucleic acid and cannot substantially bind to other nucleic acids.

TABLE 1

| SEQ | | |
|---|---|---|
| ID | | Accession No. |
| NO. | Name of gene | of miRBase |
| 1 | hsa-miR-4675 | MIMAT0019757 |
| 2 | hsa-miR-4783-3p | MIMAT0019947 |
| 3 | hsa-miR-1228-5p | MIMAT0005582 |
| 4 | hsa-miR-4532 | MIMAT0019071 |
| 5 | hsa-miR-6802-5p | MIMAT0027504 |
| 6 | hsa-miR-6784-5p | MIMAT0027468 |
| 7 | hsa-miR-3940-5p | MIMAT0019229 |
| 8 | hsa-miR-1307-3p | MIMAT0005951 |
| 9 | hsa-miR-8073 | MIMAT0031000 |
| 10 | hsa-miR-3184-5p | MIMAT0015064 |
| 11 | hsa-miR-1233-5p | MIMAT0022943 |
| 12 | hsa-miR-6088 | MIMAT0023713 |
| 13 | hsa-miR-5195-3p | MIMAT0021127 |
| 14 | hsa-miR-320b | MIMAT0005792 |
| 15 | hsa-miR-4649-5p | MIMAT0019711 |
| 16 | hsa-miR-6800-5p | MIMAT0027500 |
| 17 | hsa-miR-1343-3p | MIMAT0019776 |
| 18 | hsa-miR-4730 | MIMAT0019852 |
| 19 | hsa-miR-6885-5p | MIMAT0027670 |
| 20 | hsa-miR-5100 | MIMAT0022259 |
| 21 | hsa-miR-1203 | MIMAT0005866 |
| 22 | hsa-miR-6756-5p | MIMAT0027412 |
| 23 | hsa-miR-373-5p | MIMAT0000725 |
| 24 | hsa-miR-1268a | MIMAT0005922 |
| 25 | hsa-miR-1260b | MIMAT0015041 |
| 26 | hsa-miR-4258 | MIMAT0016879 |
| 27 | hsa-miR-4697-5p | MIMAT0019791 |
| 28 | hsa-miR-1469 | MIMAT0007347 |
| 29 | hsa-miR-4515 | MIMAT0019052 |
| 30 | hsa-miR-6861-5p | MIMAT0027623 |
| 31 | hsa-miR-6821-5p | MIMAT0027542 |
| 32 | hsa-miR-575 | MIMAT0003240 |
| 33 | hsa-miR-6805-5p | MIMAT0027510 |
| 34 | hsa-miR-4758-5p | MIMAT0019903 |
| 35 | hsa-miR-3663-3p | MIMAT0018085 |
| 36 | hsa-miR-4530 | MIMAT0019069 |
| 37 | hsa-miR-6798-5p | MIMAT0027496 |
| 38 | hsa-miR-6781-5p | MIMAT0027462 |
| 39 | hsa-miR-885-3p | MIMAT0004948 |
| 40 | hsa-miR-1273g-3p | MIMAT0022742 |
| 41 | hsa-miR-4787-3p | MIMAT0019957 |
| 42 | hsa-miR-4454 | MIMAT0018976 |
| 43 | hsa-miR-4706 | MIMAT0019806 |
| 44 | hsa-miR-1249-3p | MIMAT0005901 |
| 45 | hsa-miR-887-3p | MIMAT0004951 |
| 46 | hsa-miR-6786-5p | MIMAT0027472 |
| 47 | hsa-miR-1238-5p | MIMAT0022947 |
| 48 | hsa-miR-6749-5p | MIMAT0027398 |
| 49 | hsa-miR-6729-5p | MIMAT0027359 |
| 50 | hsa-miR-6825-5p | MIMAT0027550 |
| 51 | hsa-miR-663b | MIMAT0005867 |
| 52 | hsa-miR-6858-5p | MIMAT0027616 |
| 53 | hsa-miR-4690-5p | MIMAT0019779 |
| 54 | hsa-miR-6765-5p | MIMAT0027430 |
| 55 | hsa-miR-4710 | MIMAT0019815 |
| 56 | hsa-miR-6775-5p | MIMAT0027450 |
| 57 | hsa-miR-371a-5p | MIMAT0004687 |
| 58 | hsa-miR-6816-5p | MIMAT0027532 |
| 59 | hsa-miR-296-3p | MIMAT0004679 |
| 60 | hsa-miR-7977 | MIMAT0031180 |
| 61 | hsa-miR-8069 | MIMAT0030996 |
| 62 | hsa-miR-6515-3p | MIMAT0025487 |
| 63 | hsa-miR-4687-5p | MIMAT0019774 |
| 64 | hsa-miR-1343-5p | MIMAT0027038 |
| 65 | hsa-miR-7110-5p | MIMAT0028117 |
| 66 | hsa-miR-4525 | MIMAT0019064 |
| 67 | hsa-miR-3158-5p | MIMAT0019211 |
| 68 | hsa-miR-6787-5p | MIMAT0027474 |
| 69 | hsa-miR-614 | MIMAT0003282 |
| 70 | hsa-miR-4689 | MIMAT0019778 |
| 71 | hsa-miR-1185-2-3p | MIMAT0022713 |
| 72 | hsa-miR-1268b | MIMAT0018925 |
| 73 | hsa-miR-1228-3p | MIMAT0005583 |
| 74 | hsa-miR-1185-1-3p | MIMAT0022838 |
| 75 | hsa-miR-940 | MIMAT0004983 |
| 76 | hsa-miR-939-5p | MIMAT0004982 |
| 77 | hsa-miR-6757-5p | MIMAT0027414 |
| 78 | hsa-miR-1275 | MIMAT0005929 |
| 79 | hsa-miR-5001-5p | MIMAT0021021 |
| 80 | hsa-miR-6826-5p | MIMAT0027552 |
| 81 | hsa-miR-6765-3p | MIMAT0027431 |
| 82 | hsa-miR-3679-3p | MIMAT0018105 |
| 83 | hsa-miR-4718 | MIMAT0019831 |
| 84 | hsa-miR-4286 | MIMAT0016916 |
| 85 | hsa-miR-8059 | MIMAT0030986 |
| 86 | hsa-miR-4447 | MIMAT0018966 |
| 87 | hsa-miR-4448 | MIMAT0018967 |
| 88 | hsa-miR-658 | MIMAT0003336 |
| 89 | hsa-miR-6766-3p | MIMAT0027433 |
| 90 | hsa-miR-197-5p | MIMAT0022691 |
| 91 | hsa-miR-6887-5p | MIMAT0027674 |
| 92 | hsa-miR-6742-5p | MIMAT0027385 |
| 93 | hsa-miR-6729-3p | MIMAT0027360 |
| 94 | hsa-miR-5090 | MIMAT0021082 |
| 95 | hsa-miR-7975 | MIMAT0031178 |
| 96 | hsa-miR-4505 | MIMAT0019041 |
| 97 | hsa-miR-6889-5p | MIMAT0027678 |
| 98 | hsa-miR-4708-3p | MIMAT0019810 |
| 99 | hsa-miR-6131 | MIMAT0024615 |
| 100 | hsa-miR-1225-3p | MIMAT0005573 |
| 101 | hsa-miR-6132 | MIMAT0024616 |
| 102 | hsa-miR-4734 | MIMAT0019859 |
| 103 | hsa-miR-3194-3p | MIMAT0019218 |
| 104 | hsa-miR-638 | MIMAT0003308 |
| 105 | hsa-miR-2467-3p | MIMAT0019953 |
| 106 | hsa-miR-4728-5p | MIMAT0019849 |
| 107 | hsa-miR-5572 | MIMAT0022260 |
| 108 | hsa-miR-6789-5p | MIMAT0027478 |
| 109 | hsa-miR-8063 | MIMAT0030990 |
| 110 | hsa-miR-4429 | MIMAT0018944 |
| 111 | hsa-miR-6840-3p | MIMAT0027583 |
| 112 | hsa-miR-4476 | MIMAT0019003 |
| 113 | hsa-miR-675-5p | MIMAT0004284 |
| 114 | hsa-miR-711 | MIMAT0012734 |
| 115 | hsa-miR-6875-5p | MIMAT0027650 |
| 116 | hsa-miR-3160-5p | MIMAT0019212 |
| 117 | hsa-miR-1908-5p | MIMAT0007881 |
| 118 | hsa-miR-6726-5p | MIMAT0027353 |
| 119 | hsa-miR-1913 | MIMAT0007888 |
| 120 | hsa-miR-8071 | MIMAT0030998 |
| 121 | hsa-miR-3648 | MIMAT0018068 |
| 122 | hsa-miR-4732-5p | MIMAT0019855 |
| 123 | hsa-miR-4787-5p | MIMAT0019956 |
| 124 | hsa-miR-3917 | MIMAT0018191 |
| 125 | hsa-miR-619-5p | MIMAT0026622 |
| 126 | hsa-miR-1231 | MIMAT0005586 |
| 127 | hsa-miR-342-5p | MIMAT0004694 |
| 128 | hsa-miR-4433a-5p | MIMAT0020956 |
| 129 | hsa-miR-6766-5p | MIMAT0027432 |
| 130 | hsa-miR-4707-5p | MIMAT0019807 |
| 131 | hsa-miR-7114-5p | MIMAT0028125 |
| 132 | hsa-miR-6872-3p | MIMAT0027645 |
| 133 | hsa-miR-6780b-5p | MIMAT0027572 |
| 134 | hsa-miR-7845-5p | MIMAT0030420 |
| 135 | hsa-miR-6798-3p | MIMAT0027497 |
| 136 | hsa-miR-665 | MIMAT0004952 |
| 137 | hsa-miR-6848-5p | MIMAT0027596 |
| 138 | hsa-miR-5008-5p | MIMAT0021039 |
| 139 | hsa-miR-4294 | MIMAT0016849 |
| 140 | hsa-miR-6511a-5p | MIMAT0025478 |
| 141 | hsa-miR-4435 | MIMAT0018951 |
| 142 | hsa-miR-4747-3p | MIMAT0019883 |
| 143 | hsa-miR-6880-3p | MIMAT0027661 |
| 144 | hsa-miR-6869-5p | MIMAT0027638 |
| 145 | hsa-miR-7150 | MIMAT0028211 |
| 146 | hsa-miR-1260a | MIMAT0005911 |
| 147 | hsa-miR-6877-5p | MIMAT0027654 |
| 148 | hsa-miR-6721-5p | MIMAT0025852 |
| 149 | hsa-miR-4656 | MIMAT0019723 |
| 150 | hsa-miR-1229-5p | MIMAT0022942 |
| 151 | hsa-miR-4433a-3p | MIMAT0018949 |
| 152 | hsa-miR-4274 | MIMAT0016906 |
| 153 | hsa-miR-4419b | MIMAT0019034 |
| 154 | hsa-miR-4674 | MIMAT0019756 |
| 155 | hsa-miR-6893-5p | MIMAT0027686 |
| 156 | hsa-miR-6763-3p | MIMAT0027427 |
| 157 | hsa-miR-6762-5p | MIMAT0027424 |
| 158 | hsa-miR-6738-5p | MIMAT0027377 |
| 159 | hsa-miR-4513 | MIMAT0019050 |
| 160 | hsa-miR-6746-5p | MIMAT0027392 |
| 161 | hsa-miR-6880-5p | MIMAT0027660 |
| 162 | hsa-miR-4736 | MIMAT0019862 |
| 163 | hsa-miR-718 | MIMAT0012735 |
| 164 | hsa-miR-6717-5p | MIMAT0025846 |
| 165 | hsa-miR-7847-3p | MIMAT0030422 |
| 166 | hsa-miR-760 | MIMAT0004957 |
| 167 | hsa-miR-1199-5p | MIMAT0031119 |
| 168 | hsa-miR-6813-5p | MIMAT0027526 |
| 169 | hsa-miR-6769a-5p | MIMAT0027438 |
| 170 | hsa-miR-1193 | MIMAT0015049 |
| 171 | hsa-miR-7108-3p | MIMAT0028114 |
| 172 | hsa-miR-6741-5p | MIMAT0027383 |
| 173 | hsa-miR-4298 | MIMAT0016852 |
| 174 | hsa-miR-6796-3p | MIMAT0027493 |
| 175 | hsa-miR-4750-5p | MIMAT0019887 |
| 176 | hsa-miR-6785-5p | MIMAT0027470 |
| 177 | hsa-miR-1292-3p | MIMAT0022948 |
| 178 | hsa-miR-4749-3p | MIMAT0019886 |
| 179 | hsa-miR-6800-3p | MIMAT0027501 |
| 180 | hsa-miR-4722-5p | MIMAT0019836 |
| 181 | hsa-miR-4746-3p | MIMAT0019881 |
| 182 | hsa-miR-4450 | MIMAT0018971 |
| 183 | hsa-miR-6795-5p | MIMAT0027490 |
| 184 | hsa-miR-365a-5p | MIMAT0009199 |
| 185 | hsa-miR-498 | MIMAT0002824 |
| 186 | hsa-miR-6797-5p | MIMAT0027494 |
| 187 | hsa-miR-1470 | MIMAT0007348 |
| 188 | hsa-miR-6851-5p | MIMAT0027602 |
| 189 | hsa-miR-1247-3p | MIMAT0022721 |
| 190 | hsa-miR-5196-5p | MIMAT0021128 |
| 191 | hsa-miR-208a-5p | MIMAT0026474 |
| 192 | hsa-miR-6842-5p | MIMAT0027586 |
| 193 | hsa-miR-150-3p | MIMAT0004610 |
| 194 | hsa-miR-4534 | MIMAT0019073 |
| 195 | hsa-miR-3135b | MIMAT0018985 |
| 196 | hsa-miR-3131 | MIMAT0014996 |
| 197 | hsa-miR-4792 | MIMAT0019964 |
| 198 | hsa-miR-6510-5p | MIMAT0025476 |
| 199 | hsa-miR-504-3p | MIMAT0026612 |
| 200 | hsa-miR-3619-3p | MIMAT0019219 |
| 201 | hsa-miR-671-5p | MIMAT0003880 |
| 202 | hsa-miR-4667-5p | MIMAT0019743 |
| 203 | hsa-miR-4430 | MIMAT0018945 |
| 204 | hsa-miR-3195 | MIMAT0015079 |
| 205 | hsa-miR-3679-5p | MIMAT0018104 |
| 206 | hsa-miR-6076 | MIMAT0023701 |
| 207 | hsa-miR-6515-5p | MIMAT0025486 |
| 208 | hsa-miR-6820-5p | MIMAT0027540 |
| 209 | hsa-miR-4634 | MIMAT0019691 |
| 210 | hsa-miR-187-5p | MIMAT0004561 |
| 211 | hsa-miR-6763-5p | MIMAT0027426 |
| 212 | hsa-miR-1908-3p | MIMAT0026916 |
| 213 | hsa-miR-1181 | MIMAT0005826 |
| 214 | hsa-miR-6782-5p | MIMAT0027464 |
| 215 | hsa-miR-5010-5p | MIMAT0021043 |
| 216 | hsa-miR-6870-5p | MIMAT0027640 |
| 217 | hsa-miR-6124 | MIMAT0024597 |
| 218 | hsa-miR-1249-5p | MIMAT0032029 |
| 219 | hsa-miR-6511b-5p | MIMAT0025847 |
| 220 | hsa-miR-1254 | MIMAT0005905 |
| 221 | hsa-miR-4727-3p | MIMAT0019848 |
| 222 | hsa-miR-4259 | MIMAT0016880 |
| 223 | hsa-miR-4771 | MIMAT0019925 |
| 224 | hsa-miR-3622a-5p | MIMAT0018003 |
| 225 | hsa-miR-4480 | MIMAT0019014 |
| 226 | hsa-miR-4740-5p | MIMAT0019869 |
| 227 | hsa-miR-6777-5p | MIMAT0027454 |
| 228 | hsa-miR-6794-5p | MIMAT0027488 |
| 229 | hsa-miR-4687-3p | MIMAT0019775 |
| 230 | hsa-miR-6743-5p | MIMAT0027387 |
| 231 | hsa-miR-6771-5p | MIMAT0027442 |
| 232 | hsa-miR-3141 | MIMAT0015010 |
| 233 | hsa-miR-3162-5p | MIMAT0015036 |
| 234 | hsa-miR-4271 | MIMAT0016901 |
| 235 | hsa-miR-1227-5p | MIMAT0022941 |
| 236 | hsa-miR-4257 | MIMAT0016878 |
| 237 | hsa-miR-4270 | MIMAT0016900 |
| 238 | hsa-miR-4516 | MIMAT0019053 |
| 239 | hsa-miR-4651 | MIMAT0019715 |
| 240 | hsa-miR-4725-3p | MIMAT0019844 |
| 241 | hsa-miR-6125 | MIMAT0024598 |
| 242 | hsa-miR-6732-5p | MIMAT0027365 |
| 243 | hsa-miR-6791-5p | MIMAT0027482 |
| 244 | hsa-miR-6819-5p | MIMAT0027538 |
| 245 | hsa-miR-6891-5p | MIMAT0027682 |
| 246 | hsa-miR-7108-5p | MIMAT0028113 |
| 247 | hsa-miR-7109-5p | MIMAT0028115 |
| 248 | hsa-miR-320a | MIMAT0000510 |
| 249 | hsa-miR-663a | MIMAT0003326 |
| 250 | hsa-miR-328-5p | MIMAT0026486 |
| 251 | hsa-miR-642b-3p | MIMAT0018444 |
| 252 | hsa-miR-128-2-5p | MIMAT0031095 |
| 253 | hsa-miR-125a-3p | MIMAT0004602 |
| 254 | hsa-miR-191-5p | MIMAT0000440 |
| 255 | hsa-miR-92b-5p | MIMAT0004792 |
| 256 | hsa-miR-296-5p | MIMAT0000690 |
| 257 | hsa-miR-1246 | MIMAT0005898 |
| 258 | hsa-miR-92a-2-5p | MIMAT0004508 |
| 259 | hsa-miR-128-1-5p | MIMAT0026477 |
| 260 | hsa-miR-1290 | MIMAT0005880 |
| 261 | hsa-miR-211-3p | MIMAT0022694 |
| 262 | hsa-miR-744-5p | MIMAT0004945 |
| 263 | hsa-miR-135a-3p | MIMAT0004595 |
| 264 | hsa-miR-451a | MIMAT0001631 |
| 265 | hsa-miR-625-3p | MIMAT0004808 |
| 266 | hsa-miR-92a-3p | MIMAT0000092 |
| 267 | hsa-miR-422a | MIMAT0001339 |
| 268 | hsa-miR-642a-3p | MIMAT0020924 |
| 269 | hsa-miR-483-5p | MIMAT0004761 |
| 270 | hsa-miR-652-5p | MIMAT0022709 |
| 271 | hsa-miR-24-3p | MIMAT0000080 |
| 272 | hsa-miR-23b-3p | MIMAT0000418 |
| 273 | hsa-miR-23a-3p | MIMAT0000078 |
| 274 | hsa-miR-92b-3p | MIMAT0003218 |
| 275 | hsa-miR-22-3p | MIMAT0000077 |
| 276 | hsa-mir-4675 | MI0017306 |
| 277 | hsa-mir-4783 | MI0017428 |
| 278 | hsa-mir-1228 | MI0006318 |
| 279 | hsa-mir-4532 | MI0016899 |
| 280 | hsa-mir-6802 | MI0022647 |
| 281 | hsa-mir-6784 | MI0022629 |
| 282 | hsa-mir-3940 | MI0016597 |
| 283 | hsa-mir-1307 | MI0006444 |
| 284 | hsa-mir-8073 | MI0025909 |
| 285 | hsa-mir-3184 | MI0014226 |
| 286 | hsa-mir-1233-1 | MI0006323 |
| 287 | hsa-mir-1233-2 | MI0015973 |
| 288 | hsa-mir-6088 | MI0020365 |
| 289 | hsa-mir-5195 | MI0018174 |
| 290 | hsa-mir-320b-1 | MI0003776 |
| 291 | hsa-mir-320b-2 | MI0003839 |
| 292 | hsa-mir-4649 | MI0017276 |
| 293 | hsa-mir-6800 | MI0022645 |
| 294 | hsa-mir-1343 | MI0017320 |
| 295 | hsa-mir-4730 | MI0017367 |
| 296 | hsa-mir-6885 | MI0022732 |
| 297 | hsa-mir-5100 | MI0019116 |
| 298 | hsa-mir-1203 | MI0006335 |
| 299 | hsa-mir-6756 | MI0022601 |
| 300 | hsa-mir-373 | MI0000781 |
| 301 | hsa-mir-1268a | MI0006405 |
| 302 | hsa-mir-1260b | MI0014197 |
| 303 | hsa-mir-4258 | MI0015857 |
| 304 | hsa-mir-4697 | MI0017330 |
| 305 | hsa-mir-1469 | MI0007074 |
| 306 | hsa-mir-4515 | MI0016881 |
| 307 | hsa-mir-6861 | MI0022708 |
| 308 | hsa-mir-6821 | MI0022666 |
| 309 | hsa-mir-575 | MI0003582 |
| 310 | hsa-mir-6805 | MI0022650 |
| 311 | hsa-mir-4758 | MI0017399 |
| 312 | hsa-mir-3663 | MI0016064 |
| 313 | hsa-mir-4530 | MI0016897 |
| 314 | hsa-mir-6798 | MI0022643 |
| 315 | hsa-mir-6781 | MI0022626 |
| 316 | hsa-mir-885 | MI0005560 |
| 317 | hsa-mir-1273g | MI0018003 |
| 318 | hsa-mir-4787 | MI0017434 |
| 319 | hsa-mir-4454 | MI0016800 |
| 320 | hsa-mir-4706 | MI0017339 |
| 321 | hsa-mir-1249 | MI0006384 |
| 322 | hsa-mir-887 | MI0005562 |
| 323 | hsa-mir-6786 | MI0022631 |
| 324 | hsa-mir-1238 | MI0006328 |
| 325 | hsa-mir-6749 | MI0022594 |
| 326 | hsa-mir-6729 | MI0022574 |
| 327 | hsa-mir-6825 | MI0022670 |
| 328 | hsa-mir-663b | MI0006336 |
| 329 | hsa-mir-6858 | MI0022704 |
| 330 | hsa-mir-4690 | MI0017323 |
| 331 | hsa-mir-6765 | MI0022610 |
| 332 | hsa-mir-4710 | MI0017344 |
| 333 | hsa-mir-6775 | MI0022620 |
| 334 | hsa-mir-371a | MI0000779 |
| 335 | hsa-mir-6816 | MI0022661 |
| 336 | hsa-mir-296 | MI0000747 |
| 337 | hsa-mir-7977 | MI0025753 |
| 338 | hsa-mir-8069-1 | MI0025905 |
| 339 | hsa-mir-8069-2 | MI0031519 |
| 340 | hsa-mir-6515 | MI0022227 |
| 341 | hsa-mir-4687 | MI0017319 |
| 342 | hsa-mir-7110 | MI0022961 |
| 343 | hsa-mir-4525 | MI0016892 |
| 344 | hsa-mir-3158-1 | MI0014186 |
| 345 | hsa-mir-3158-2 | MI0014187 |
| 346 | hsa-mir-6787 | MI0022632 |
| 347 | hsa-mir-614 | MI0003627 |
| 348 | hsa-mir-4689 | MI0017322 |
| 349 | hsa-mir-1185-2 | MI0003821 |
| 350 | hsa-mir-1268b | MI0016748 |
| 351 | hsa-mir-1185-1 | MI0003844 |
| 352 | hsa-mir-940 | MI0005762 |
| 353 | hsa-mir-939 | MI0005761 |
| 354 | hsa-mir-6757 | MI0022602 |
| 355 | hsa-mir-1275 | MI0006415 |
| 356 | hsa-mir-5001 | MI0017867 |
| 357 | hsa-mir-6826 | MI0022671 |
| 358 | hsa-mir-3679 | MI0016080 |
| 359 | hsa-mir-4718 | MI0017353 |
| 360 | hsa-mir-4286 | MI0015894 |
| 361 | hsa-mir-8059 | MI0025895 |
| 362 | hsa-mir-4447 | MI0016790 |
| 363 | hsa-mir-4448 | MI0016791 |
| 364 | hsa-mir-658 | MI0003682 |
| 365 | hsa-mir-6766 | MI0022611 |
| 366 | hsa-mir-197 | MI0000239 |
| 367 | hsa-mir-6887 | MI0022734 |
| 368 | hsa-mir-6742 | MI0022587 |
| 369 | hsa-mir-5090 | MI0017979 |
| 370 | hsa-mir-7975 | MI0025751 |
| 371 | hsa-mir-4505 | MI0016868 |
| 372 | hsa-mir-6889 | MI0022736 |
| 373 | hsa-mir-4708 | MI0017341 |
| 374 | hsa-mir-6131 | MI0021276 |
| 375 | hsa-mir-1225 | MI0006311 |
| 376 | hsa-mir-6132 | MI0021277 |
| 377 | hsa-mir-4734 | MI0017371 |
| 378 | hsa-mir-3194 | MI0014239 |
| 379 | hsa-mir-638 | MI0003653 |
| 380 | hsa-mir-2467 | MI0017432 |
| 381 | hsa-mir-4728 | MI0017365 |
| 382 | hsa-mir-5572 | MI0019117 |
| 383 | hsa-mir-6789 | MI0022634 |
| 384 | hsa-mir-8063 | MI0025899 |
| 385 | hsa-mir-4429 | MI0016768 |
| 386 | hsa-mir-6840 | MI0022686 |
| 387 | hsa-mir-4476 | MI0016828 |
| 388 | hsa-mir-675 | MI0005416 |
| 389 | hsa-mir-711 | MI0012488 |
| 390 | hsa-mir-6875 | MI0022722 |
| 391 | hsa-mir-3160-1 | MI0014189 |
| 392 | hsa-mir-3160-2 | MI0014190 |
| 393 | hsa-mir-1908 | MI0008329 |
| 394 | hsa-mir-6726 | MI0022571 |
| 395 | hsa-mir-1913 | MI0008334 |
| 396 | hsa-mir-8071-1 | MI0025907 |
| 397 | hsa-mir-8071-2 | MI0026417 |
| 398 | hsa-mir-3648-1 | MI0016048 |
| 399 | hsa-mir-3648-2 | MI0031512 |
| 400 | hsa-mir-4732 | MI0017369 |
| 401 | hsa-mir-3917 | MI0016423 |
| 402 | hsa-mir-619 | MI0003633 |
| 403 | hsa-mir-1231 | MI0006321 |
| 404 | hsa-mir-342 | MI0000805 |
| 405 | hsa-mir-4433a | MI0016773 |
| 406 | hsa-mir-4707 | MI0017340 |
| 407 | hsa-mir-7114 | MI0022965 |
| 408 | hsa-mir-6872 | MI0022719 |
| 409 | hsa-mir-6780b | MI0022681 |
| 410 | hsa-mir-7845 | MI0025515 |
| 411 | hsa-mir-665 | MI0005563 |
| 412 | hsa-mir-6848 | MI0022694 |
| 413 | hsa-mir-5008 | MI0017876 |
| 414 | hsa-mir-4294 | MI0015827 |
| 415 | hsa-mir-6511a-1 | MI0022223 |
| 416 | hsa-mir-6511a-2 | MI0023564 |
| 417 | hsa-mir-6511a-3 | MI0023565 |
| 418 | hsa-mir-6511a-4 | MI0023566 |
| 419 | hsa-mir-4435-1 | MI0016775 |
| 420 | hsa-mir-4435-2 | MI0016777 |
| 421 | hsa-mir-4747 | MI0017386 |
| 422 | hsa-mir-6880 | MI0022727 |
| 423 | hsa-mir-6869 | MI0022716 |
| 424 | hsa-mir-7150 | MI0023610 |
| 425 | hsa-mir-1260a | MI0006394 |
| 426 | hsa-mir-6877 | MI0022724 |
| 427 | hsa-mir-6721 | MI0022556 |
| 428 | hsa-mir-4656 | MI0017284 |
| 429 | hsa-mir-1229 | MI0006319 |
| 430 | hsa-mir-4274 | MI0015884 |
| 431 | hsa-mir-4419b | MI0016861 |
| 432 | hsa-mir-4674 | MI0017305 |
| 433 | hsa-mir-6893 | MI0022740 |
| 434 | hsa-mir-6763 | MI0022608 |
| 435 | hsa-mir-6762 | MI0022607 |
| 436 | hsa-mir-6738 | MI0022583 |
| 437 | hsa-mir-4513 | MI0016879 |
| 438 | hsa-mir-6746 | MI0022591 |
| 439 | hsa-mir-4736 | MI0017373 |
| 440 | hsa-mir-718 | MI0012489 |
| 441 | hsa-mir-6717 | MI0022551 |
| 442 | hsa-mir-7847 | MI0025517 |
| 443 | hsa-mir-760 | MI0005567 |
| 444 | hsa-mir-1199 | MI0020340 |
| 445 | hsa-mir-6813 | MI0022658 |
| 446 | hsa-mir-6769a | MI0022614 |
| 447 | hsa-mir-1193 | MI0014205 |
| 448 | hsa-mir-7108 | MI0022959 |
| 449 | hsa-mir-6741 | MI0022586 |
| 450 | hsa-mir-4298 | MI0015830 |
| 451 | hsa-mir-6796 | MI0022641 |
| 452 | hsa-mir-4750 | MI0017389 |
| 453 | hsa-mir-6785 | MI0022630 |
| 454 | hsa-mir-1292 | MI0006433 |
| 455 | hsa-mir-4749 | MI0017388 |
| 456 | hsa-mir-4722 | MI0017357 |
| 457 | hsa-mir-4746 | MI0017385 |
| 458 | hsa-mir-4450 | MI0016795 |
| 459 | hsa-mir-6795 | MI0022640 |
| 460 | hsa-mir-365a | MI0000767 |
| 461 | hsa-mir-498 | MI0003142 |
| 462 | hsa-mir-6797 | MI0022642 |
| 463 | hsa-mir-1470 | MI0007075 |
| 464 | hsa-mir-6851 | MI0022697 |
| 465 | hsa-mir-1247 | MI0006382 |
| 466 | hsa-mir-5196 | MI0018175 |
| 467 | hsa-mir-208a | MI0000251 |
| 468 | hsa-mir-6842 | MI0022688 |
| 469 | hsa-mir-150 | MI0000479 |
| 470 | hsa-mir-4534 | MI0016901 |
| 471 | hsa-mir-3135b | MI0016809 |
| 472 | hsa-mir-3131 | MI0014151 |
| 473 | hsa-mir-4792 | MI0017439 |
| 474 | hsa-mir-6510 | MI0022222 |
| 475 | hsa-mir-504 | MI0003189 |
| 476 | hsa-mir-3619 | MI0016009 |
| 477 | hsa-mir-671 | MI0003760 |
| 478 | hsa-mir-4667 | MI0017297 |
| 479 | hsa-mir-4430 | MI0016769 |
| 480 | hsa-mir-3195 | MI0014240 |
| 481 | hsa-mir-6076 | MI0020353 |
| 482 | hsa-mir-6820 | MI0022665 |
| 483 | hsa-mir-4634 | MI0017261 |
| 484 | hsa-mir-187 | MI0000274 |
| 485 | hsa-mir-1181 | MI0006274 |
| 486 | hsa-mir-6782 | MI0022627 |
| 487 | hsa-mir-5010 | MI0017878 |
| 488 | hsa-mir-6870 | MI0022717 |
| 489 | hsa-mir-6124 | MI0021258 |
| 490 | hsa-mir-6511b-1 | MI0022552 |
| 491 | hsa-mir-6511b-2 | MI0023431 |
| 492 | hsa-mir-1254-1 | MI0006388 |
| 493 | hsa-mir-1254-2 | MI0016747 |
| 494 | hsa-mir-4727 | MI0017364 |
| 495 | hsa-mir-4259 | MI0015858 |
| 496 | hsa-mir-4771-1 | MI0017412 |
| 497 | hsa-mir-4771-2 | MI0017413 |
| 498 | hsa-mir-3622a | MI0016013 |
| 499 | hsa-mir-4480 | MI0016841 |
| 500 | hsa-mir-4740 | MI0017378 |
| 501 | hsa-mir-6777 | MI0022622 |
| 502 | hsa-mir-6794 | MI0022639 |
| 503 | hsa-mir-6743 | MI0022588 |
| 504 | hsa-mir-6771 | MI0022616 |
| 505 | hsa-mir-3141 | MI0014165 |
| 506 | hsa-mir-3162 | MI0014192 |
| 507 | hsa-mir-4271 | MI0015879 |
| 508 | hsa-mir-1227 | MI0006316 |
| 509 | hsa-mir-4257 | MI0015856 |
| 510 | hsa-mir-4270 | MI0015878 |
| 511 | hsa-mir-4516 | MI0016882 |
| 512 | hsa-mir-4651 | MI0017279 |
| 513 | hsa-mir-4725 | MI0017362 |
| 514 | hsa-mir-6125 | MI0021259 |
| 515 | hsa-mir-6732 | MI0022577 |
| 516 | hsa-mir-6791 | MI0022636 |
| 517 | hsa-mir-6819 | MI0022664 |
| 518 | hsa-mir-6891 | MI0022738 |
| 519 | hsa-mir-7109 | MI0022960 |
| 520 | hsa-mir-320a | MI0000542 |
| 521 | hsa-mir-663a | MI0003672 |
| 522 | hsa-mir-328 | MI0000804 |
| 523 | hsa-mir-642b | MI0016685 |
| 524 | hsa-mir-128-2 | MI0000727 |
| 525 | hsa-mir-125a | MI0000469 |
| 526 | hsa-mir-191 | MI0000465 |
| 527 | hsa-mir-92b | MI0003560 |
| 528 | hsa-mir-1246 | MI0006381 |
| 529 | hsa-mir-92a-2 | MI0000094 |
| 530 | hsa-mir-128-1 | MI0000447 |
| 531 | hsa-mir-1290 | MI0006352 |
| 532 | hsa-mir-211 | MI0000287 |
| 533 | hsa-mir-744 | MI0005559 |
| 534 | hsa-mir-135a-1 | MI0000452 |
| 535 | hsa-mir-451a | MI0001729 |
| 536 | hsa-mir-625 | MI0003639 |
| 537 | hsa-mir-92a-1 | MI0000093 |
| 538 | hsa-mir-422a | MI0001444 |
| 539 | hsa-mir-642a | MI0003657 |
| 540 | hsa-mir-483 | MI0002467 |
| 541 | hsa-mir-652 | MI0003667 |
| 542 | hsa-mir-24-1 | MI0000080 |
| 543 | hsa-mir-24-2 | MI0000081 |
| 544 | hsa-mir-23b | MI0000439 |
| 545 | hsa-mir-23a | MI0000079 |
| 546 | hsa-mir-22 | MI0000078 |
| 547 | isomiR Example 1 of SEQ ID NO: 2 | — |
| 548 | isomiR Example 2 of SEQ ID NO: 2 | — |
| 549 | isomiR Example 1 of SEQ ID NO: 3 | — |
| 550 | isomiR Example 2 of SEQ ID NO: 3 | — |
| 551 | isomiR Example 1 of SEQ ID NO: 4 | — |
| 552 | isomiR Example 2 of SEQ ID NO: 4 | — |
| 553 | isomiR Example 1 of SEQ ID NO: 7 | — |
| 554 | isomiR Example 1 of SEQ ID NO: 8 | — |
| 555 | isomiR Example 2 of SEQ ID NO: 8 | — |
| 556 | isomiR Example 1 of SEQ ID NO: 11 | — |
| 557 | isomiR Example 2 of SEQ ID NO: 11 | — |
| 558 | isomiR Example 1 of SEQ ID NO: 12 | — |
| 559 | isomiR Example 1 of SEQ ID NO: 13 | — |
| 560 | isomiR Example 1 of SEQ ID NO: 14 | — |
| 561 | isomiR Example 2 of SEQ ID NO: 14 | — |
| 562 | isomiR Example 1 of SEQ ID NO: 15 | — |
| 563 | isomiR Example 1 of SEQ ID NO: 17 | — |
| 564 | isomiR Example 2 of SEQ ID NO: 17 | — |
| 565 | isomiR Example 1 of SEQ ID NO: 18 | — |
| 566 | isomiR Example 2 of SEQ ID NO: 18 | — |
| 567 | isomiR Example 1 of SEQ ID NO: 20 | — |
| 568 | isomiR Example 2 of SEQ ID NO: 20 | — |
| 569 | isomiR Example 1 of SEQ ID NO: 23 | — |
| 570 | isomiR Example 1 of SEQ ID NO: 24 | — |
| 571 | isomiR Example 2 of SEQ ID NO: 24 | — |
| 572 | isomiR Example 1 of SEQ ID NO: 25 | — |
| 573 | isomiR Example 2 of SEQ ID NO: 25 | — |
| 574 | isomiR Example 1 of SEQ ID NO: 29 | — |
| 575 | isomiR Example 2 of SEQ ID NO: 29 | — |
| 576 | isomiR Example 1 of SEQ ID NO: 34 | — |
| 577 | isomiR Example 2 of SEQ ID NO: 34 | — |
| 578 | isomiR Example 1 of SEQ ID NO: 36 | — |
| 579 | isomiR Example 2 of SEQ ID NO: 36 | — |
| 580 | isomiR Example 1 of SEQ ID NO: 39 | — |
| 581 | isomiR Example 2 of SEQ ID NO: 39 | — |
| 582 | isomiR Example 1 of SEQ ID NO: 40 | — |
| 583 | isomiR Example 2 of SEQ ID NO: 40 | — |
| 584 | isomiR Example 1 of SEQ ID NO: 41 | — |
| 585 | isomiR Example 2 of SEQ ID NO: 41 | — |
| 586 | isomiR Example 1 of SEQ ID NO: 42 | — |
| 587 | isomiR Example 2 of SEQ ID NO: 42 | — |
| 588 | isomiR Example 1 of SEQ ID NO: 43 | — |
| 589 | isomiR Example 2 of SEQ ID NO: 43 | — |
| 590 | isomiR Example 1 of SEQ ID NO: 44 | — |
| 591 | isomiR Example 2 of SEQ ID NO: 44 | — |
| 592 | isomiR Example 1 of SEQ ID NO: 45 | — |
| 593 | isomiR Example 2 of SEQ ID NO: 45 | — |
| 594 | isomiR Example 1 of SEQ ID NO: 51 | — |
| 595 | isomiR Example 2 of SEQ ID NO: 51 | — |
| 596 | isomiR Example 1 of SEQ ID NO: 53 | — |
| 597 | isomiR Example 2 of SEQ ID NO: 53 | — |
| 598 | isomiR Example 1 of SEQ ID NO: 55 | — |
| 599 | isomiR Example 2 of SEQ ID NO: 55 | — |
| 600 | isomiR Example 1 of SEQ ID NO: 57 | — |
| 601 | isomiR Example 2 of SEQ ID NO: 57 | — |
| 602 | isomiR Example 1 of SEQ ID NO: 59 | — |
| 603 | isomiR Example 2 of SEQ ID NO: 59 | — |
| 604 | isomiR Example 1 of SEQ ID NO: 62 | — |
| 605 | isomiR Example 2 of SEQ ID NO: 62 | — |
| 606 | isomiR Example 1 of SEQ ID NO: 63 | — |
| 607 | isomiR Example 2 of SEQ ID NO: 63 | — |
| 608 | isomiR Example 1 of SEQ ID NO: 66 | — |
| 609 | isomiR Example 2 of SEQ ID NO: 66 | — |
| 610 | isomiR Example 1 of SEQ ID NO: 67 | — |
| 611 | isomiR Example 2 of SEQ ID NO: 67 | — |
| 612 | isomiR Example 1 of SEQ ID NO: 69 | — |
| 613 | isomiR Example 2 of SEQ ID NO: 69 | — |
| 614 | isomiR Example 1 of SEQ ID NO: 70 | — |
| 615 | isomiR Example 2 of SEQ ID NO: 70 | — |
| 616 | isomiR Example 1 of SEQ ID NO: 71 | — |
| 617 | isomiR Example 2 of SEQ ID NO: 71 | — |
| 618 | isomiR Example 1 of SEQ ID NO: 72 | — |
| 619 | isomiR Example 2 of SEQ ID NO: 72 | — |
| 620 | isomiR Example 1 of SEQ ID NO: 73 | — |
| 621 | isomiR Example 2 of SEQ ID NO: 73 | — |
| 622 | isomiR Example 1 of SEQ ID NO: 74 | — |
| 623 | isomiR Example 1 of SEQ ID NO: 75 | — |
| 624 | isomiR Example 2 of SEQ ID NO: 75 | — |
| 625 | isomiR Example 1 of SEQ ID NO: 76 | — |
| 626 | isomiR Example 2 of SEQ ID NO: 76 | — |
| 627 | isomiR Example 1 of SEQ ID NO: 78 | — |
| 628 | isomiR Example 2 of SEQ ID NO: 78 | — |
| 629 | isomiR Example 1 of SEQ ID NO: 79 | — |
| 630 | isomiR Example 2 of SEQ ID NO: 79 | — |
| 631 | isomiR Example 1 of SEQ ID NO: 82 | — |
| 632 | isomiR Example 1 of SEQ ID NO: 84 | — |
| 633 | isomiR Example 2 of SEQ ID NO: 84 | — |
| 634 | isomiR Example 1 of SEQ ID NO: 87 | — |
| 635 | isomiR Example 2 of SEQ ID NO: 87 | — |
| 636 | isomiR Example 1 of SEQ ID NO: 88 | — |
| 637 | isomiR Example 2 of SEQ ID NO: 88 | — |
| 638 | isomiR Example 1 of SEQ ID NO: 90 | — |
| 639 | isomiR Example 2 of SEQ ID NO: 90 | — |
| 640 | isomiR Example 1 of SEQ ID NO: 93 | — |
| 641 | isomiR Example 1 of SEQ ID NO: 94 | — |
| 642 | isomiR Example 2 of SEQ ID NO: 94 | — |
| 643 | isomiR Example 1 of SEQ ID NO: 95 | — |
| 644 | isomiR Example 1 of SEQ ID NO: 96 | — |
| 645 | isomiR Example 2 of SEQ ID NO: 96 | — |
| 646 | isomiR Example 1 of SEQ ID NO: 98 | — |
| 647 | isomiR Example 2 of SEQ ID NO: 98 | — |
| 648 | isomiR Example 1 of SEQ ID NO: 99 | — |
| 649 | isomiR Example 1 of SEQ ID NO: 101 | — |
| 650 | isomiR Example 2 of SEQ ID NO: 101 | — |
| 651 | isomiR Example 1 of SEQ ID NO: 102 | — |
| 652 | isomiR Example 2 of SEQ ID NO: 102 | — |
| 653 | isomiR Example 1 of SEQ ID NO: 103 | — |
| 654 | isomiR Example 2 of SEQ ID NO: 103 | — |
| 655 | isomiR Example 1 of SEQ ID NO: 104 | — |
| 656 | isomiR Example 2 of SEQ ID NO: 104 | — |
| 657 | isomiR Example 1 of SEQ ID NO: 105 | — |
| 658 | isomiR Example 2 of SEQ ID NO: 105 | — |
| 659 | isomiR Example 1 of SEQ ID NO: 106 | — |
| 660 | isomiR Example 1 of SEQ ID NO: 107 | — |
| 661 | isomiR Example 1 of SEQ ID NO: 110 | — |
| 662 | isomiR Example 2 of SEQ ID NO: 110 | — |
| 663 | isomiR Example 1 of SEQ ID NO: 112 | — |
| 664 | isomiR Example 2 of SEQ ID NO: 112 | — |
| 665 | isomiR Example 1 of SEQ ID NO: 113 | — |
| 666 | isomiR Example 2 of SEQ ID NO: 113 | — |
| 667 | isomiR Example 1 of SEQ ID NO: 114 | — |
| 668 | isomiR Example 1 of SEQ ID NO: 117 | — |
| 669 | isomiR Example 2 of SEQ ID NO: 117 | — |
| 670 | isomiR Example 1 of SEQ ID NO: 119 | — |
| 671 | isomiR Example 2 of SEQ ID NO: 119 | — |
| 672 | isomiR Example 1 of SEQ ID NO: 121 | — |
| 673 | isomiR Example 2 of SEQ ID NO: 121 | — |
| 674 | isomiR Example 1 of SEQ ID NO: 122 | — |
| 675 | isomiR Example 2 of SEQ ID NO: 122 | — |
| 676 | isomiR Example 1 of SEQ ID NO: 123 | — |
| 677 | isomiR Example 2 of SEQ ID NO: 123 | — |
| 678 | isomiR Example 1 of SEQ ID NO: 124 | — |
| 679 | isomiR Example 2 of SEQ ID NO: 124 | — |
| 680 | isomiR Example 1 of SEQ ID NO: 125 | — |
| 681 | isomiR Example 2 of SEQ ID NO: 125 | — |
| 682 | isomiR Example 1 of SEQ ID NO: 127 | — |
| 683 | isomiR Example 2 of SEQ ID NO: 127 | — |
| 684 | isomiR Example 1 of SEQ ID NO: 128 | — |
| 685 | isomiR Example 1 of SEQ ID NO: 130 | — |
| 686 | isomiR Example 2 of SEQ ID NO: 130 | — |
| 687 | isomiR Example 1 of SEQ ID NO: 136 | — |
| 688 | isomiR Example 2 of SEQ ID NO: 136 | — |
| 689 | isomiR Example 1 of SEQ ID NO: 140 | — |
| 690 | isomiR Example 2 of SEQ ID NO: 140 | — |
| 691 | isomiR Example 1 of SEQ ID NO: 141 | — |
| 692 | isomiR Example 2 of SEQ ID NO: 141 | — |
| 693 | isomiR Example 1 of SEQ ID NO: 144 | — |
| 694 | isomiR Example 1 of SEQ ID NO: 146 | — |
| 695 | isomiR Example 2 of SEQ ID NO: 146 | — |
| 696 | isomiR Example 1 of SEQ ID NO: 148 | — |
| 697 | isomiR Example 2 of SEQ ID NO: 148 | — |
| 698 | isomiR Example 1 of SEQ ID NO: 151 | — |
| 699 | isomiR Example 2 of SEQ ID NO: 151 | — |
| 700 | isomiR Example 1 of SEQ ID NO: 153 | — |
| 701 | isomiR Example 2 of SEQ ID NO: 153 | — |
| 702 | isomiR Example 1 of SEQ ID NO: 154 | — |
| 703 | isomiR Example 2 of SEQ ID NO: 154 | — |
| 704 | isomiR Example 1 of SEQ ID NO: 159 | — |
| 705 | isomiR Example 2 of SEQ ID NO: 159 | — |
| 706 | isomiR Example 1 of SEQ ID NO: 163 | — |
| 707 | isomiR Example 1 of SEQ ID NO: 164 | — |
| 708 | isomiR Example 2 of SEQ ID NO: 164 | — |
| 709 | isomiR Example 1 of SEQ ID NO: 166 | — |
| 710 | isomiR Example 2 of SEQ ID NO: 166 | — |
| 711 | isomiR Example 1 of SEQ ID NO: 170 | — |
| 712 | isomiR Example 2 of SEQ ID NO: 170 | — |
| 713 | isomiR Example 1 of SEQ ID NO: 173 | — |
| 714 | isomiR Example 1 of SEQ ID NO: 175 | — |
| 715 | isomiR Example 2 of SEQ ID NO: 175 | — |
| 716 | isomiR Example 1 of SEQ ID NO: 179 | — |
| 717 | isomiR Example 1 of SEQ ID NO: 180 | — |
| 718 | isomiR Example 2 of SEQ ID NO: 180 | — |
| 719 | isomiR Example 1 of SEQ ID NO: 182 | — |
| 720 | isomiR Example 1 of SEQ ID NO: 184 | — |
| 721 | isomiR Example 2 of SEQ ID NO: 184 | — |
| 722 | isomiR Example 1 of SEQ ID NO: 185 | — |
| 723 | isomiR Example 2 of SEQ ID NO: 185 | — |
| 724 | isomiR Example 1 of SEQ ID NO: 187 | — |
| 725 | isomiR Example 1 of SEQ ID NO: 189 | — |
| 726 | isomiR Example 2 of SEQ ID NO: 189 | — |
| 727 | isomiR Example 1 of SEQ ID NO: 190 | — |
| 728 | isomiR Example 2 of SEQ ID NO: 190 | — |
| 729 | isomiR Example 1 of SEQ ID NO: 193 | — |
| 730 | isomiR Example 2 of SEQ ID NO: 193 | — |
| 731 | isomiR Example 1 of SEQ ID NO: 195 | — |
| 732 | isomiR Example 2 of SEQ ID NO: 195 | — |
| 733 | isomiR Example 1 of SEQ ID NO: 196 | — |
| 734 | isomiR Example 2 of SEQ ID NO: 196 | — |
| 735 | isomiR Example 1 of SEQ ID NO: 197 | — |
| 736 | isomiR Example 2 of SEQ ID NO: 197 | — |
| 737 | isomiR Example 1 of SEQ ID NO: 198 | — |
| 738 | isomiR Example 2 of SEQ ID NO: 198 | — |
| 739 | isomiR Example 1 of SEQ ID NO: 199 | — |
| 740 | isomiR Example 2 of SEQ ID NO: 199 | — |
| 741 | isomiR Example 1 of SEQ ID NO: 201 | — |
| 742 | isomiR Example 2 of SEQ ID NO: 201 | — |
| 743 | isomiR Example 1 of SEQ ID NO: 202 | — |
| 744 | isomiR Example 2 of SEQ ID NO: 202 | — |
| 745 | isomiR Example 1 of SEQ ID NO: 203 | — |
| 746 | isomiR Example 2 of SEQ ID NO: 203 | — |
| 747 | isomiR Example 1 of SEQ ID NO: 204 | — |
| 748 | isomiR Example 2 of SEQ ID NO: 204 | — |
| 749 | isomiR Example 1 of SEQ ID NO: 205 | — |
| 750 | isomiR Example 2 of SEQ ID NO: 205 | — |
| 751 | isomiR Example 1 of SEQ ID NO: 207 | — |
| 752 | isomiR Example 2 of SEQ ID NO: 207 | — |
| 753 | isomiR Example 1 of SEQ ID NO: 209 | — |
| 754 | isomiR Example 1 of SEQ ID NO: 210 | — |
| 755 | isomiR Example 2 of SEQ ID NO: 210 | — |
| 756 | isomiR Example 1 of SEQ ID NO: 212 | — |
| 757 | isomiR Example 2 of SEQ ID NO: 212 | — |
| 758 | isomiR Example 1 of SEQ ID NO: 213 | — |
| 759 | isomiR Example 2 of SEQ ID NO: 213 | — |
| 760 | isomiR Example 1 of SEQ ID NO: 215 | — |
| 761 | isomiR Example 2 of SEQ ID NO: 215 | — |
| 762 | isomiR Example 1 of SEQ ID NO: 217 | — |
| 763 | isomiR Example 2 of SEQ ID NO: 217 | — |
| 764 | isomiR Example 1 of SEQ ID NO: 218 | — |
| 765 | isomiR Example 2 of SEQ ID NO: 218 | — |
| 766 | isomiR Example 1 of SEQ ID NO: 219 | — |
| 767 | isomiR Example 2 of SEQ ID NO: 219 | — |
| 768 | isomiR Example 1 of SEQ ID NO: 220 | — |
| 769 | isomiR Example 2 of SEQ ID NO: 220 | — |
| 770 | isomiR Example 1 of SEQ ID NO: 221 | — |
| 771 | isomiR Example 2 of SEQ ID NO: 221 | — |
| 772 | isomiR Example 1 of SEQ ID NO: 223 | — |
| 773 | isomiR Example 2 of SEQ ID NO: 223 | — |
| 774 | isomiR Example 1 of SEQ ID NO: 224 | — |
| 775 | isomiR Example 2 of SEQ ID NO: 224 | — |
| 776 | isomiR Example 1 of SEQ ID NO: 229 | — |
| 777 | isomiR Example 2 of SEQ ID NO: 229 | — |
| 778 | isomiR Example 1 of SEQ ID NO: 232 | — |
| 779 | isomiR Example 2 of SEQ ID NO: 232 | — |
| 780 | isomiR Example 1 of SEQ ID NO: 233 | — |
| 781 | isomiR Example 2 of SEQ ID NO: 233 | — |
| 782 | isomiR Example 1 of SEQ ID NO: 234 | — |
| 783 | isomiR Example 1 of SEQ ID NO: 238 | — |
| 784 | isomiR Example 2 of SEQ ID NO: 238 | — |
| 785 | isomiR Example 1 of SEQ ID NO: 239 | — |
| 786 | isomiR Example 2 of SEQ ID NO: 239 | — |
| 787 | isomiR Example 1 of SEQ ID NO: 240 | — |
| 788 | isomiR Example 2 of SEQ ID NO: 240 | — |
| 789 | isomiR Example 1 of SEQ ID NO: 241 | — |
| 790 | isomiR Example 2 of SEQ ID NO: 241 | — |
| 791 | isomiR Example 1 of SEQ ID NO: 248 | — |
| 792 | isomiR Example 2 of SEQ ID NO: 248 | — |
| 793 | isomiR Example 1 of SEQ ID NO: 249 | — |
| 794 | isomiR Example 2 of SEQ ID NO: 249 | — |
| 795 | isomiR Example 1 of SEQ ID NO: 250 | — |
| 796 | isomiR Example 2 of SEQ ID NO: 250 | — |
| 797 | isomiR Example 1 of SEQ ID NO: 251 | — |
| 798 | isomiR Example 2 of SEQ ID NO: 251 | — |
| 799 | isomiR Example 1 of SEQ ID NO: 252 | — |
| 800 | isomiR Example 2 of SEQ ID NO: 252 | — |
| 801 | isomiR Example 1 of SEQ ID NO: 253 | — |
| 802 | isomiR Example 2 of SEQ ID NO: 253 | — |
| 803 | isomiR Example 1 of SEQ ID NO: 254 | — |
| 804 | isomiR Example 2 of SEQ ID NO: 254 | — |
| 805 | isomiR Example 1 of SEQ ID NO: 255 | — |
| 806 | isomiR Example 2 of SEQ ID NO: 255 | — |
| 807 | isomiR Example 1 of SEQ ID NO: 256 | — |
| 808 | isomiR Example 2 of SEQ ID NO: 256 | — |
| 809 | isomiR Example 1 of SEQ ID NO: 257 | — |
| 810 | isomiR Example 2 of SEQ ID NO: 257 | — |
| 811 | isomiR Example 1 of SEQ ID NO: 258 | — |
| 812 | isomiR Example 2 of SEQ ID NO: 258 | — |
| 813 | isomiR Example 1 of SEQ ID NO: 259 | — |
| 814 | isomiR Example 2 of SEQ ID NO: 259 | — |
| 815 | isomiR Example 1 of SEQ ID NO: 260 | — |
| 816 | isomiR Example 2 of SEQ ID NO: 260 | — |
| 817 | isomiR Example 1 of SEQ ID NO: 261 | — |
| 818 | isomiR Example 2 of SEQ ID NO: 261 | — |
| 819 | isomiR Example 1 of SEQ ID NO: 262 | — |
| 820 | isomiR Example 2 of SEQ ID NO: 262 | — |
| 821 | isomiR Example 1 of SEQ ID NO: 263 | — |
| 822 | isomiR Example 2 of SEQ ID NO: 263 | — |
| 823 | isomiR Example 1 of SEQ ID NO: 264 | — |
| 824 | isomiR Example 2 of SEQ ID NO: 264 | — |
| 825 | isomiR Example 1 of SEQ ID NO: 265 | — |
| 826 | isomiR Example 2 of SEQ ID NO: 265 | — |
| 827 | isomiR Example 1 of SEQ ID NO: 266 | — |
| 828 | isomiR Example 2 of SEQ ID NO: 266 | — |
| 829 | isomiR Example 1 of SEQ ID NO: 268 | — |
| 830 | isomiR Example 1 of SEQ ID NO: 269 | — |
| 831 | isomiR Example 2 of SEQ ID NO: 269 | — |
| 832 | isomiR Example 1 of SEQ ID NO: 270 | — |
| 833 | isomiR Example 2 of SEQ ID NO: 270 | — |
| 834 | isomiR Example 1 of SEQ ID NO: 271 | — |
| 835 | isomiR Example 2 of SEQ ID NO: 271 | — |
| 836 | isomiR Example 1 of SEQ ID NO: 272 | — |
| 837 | isomiR Example 2 of SEQ ID NO: 272 | — |
| 838 | isomiR Example 1 of SEQ ID NO: 273 | — |
| 839 | isomiR Example 1 of SEQ ID NO: 274 | — |
| 840 | isomiR Example 2 of SEQ ID NO: 274 | — |
| 841 | isomiR Example 1 of SEQ ID NO: 275 | — |
| 842 | isomiR Example 2 of SEQ ID NO: 275 | — |

### Advantageous Effect of Invention

According to the present invention, ovarian tumor can be detected easily and in high accuracy. For example, the presence or absence of ovarian tumor in patients can be easily detected by using, as indicators, the determined expression levels of one to several miRNAs in blood, serum, and/or plasma of the patients, which can be collected with limited invasiveness.

The present specification encompasses the contents disclosed in Japanese Patent Application No. 2017-090799 from which the present application claims priority.

### BRIEF DESCRIPTION OF THE DRAWINGS

FIG. 1 This figure shows the relationship between the nucleotide sequences of hsa-miR-4433a-5p represented by SEQ ID NO: 128 and hsa-miR-4433a-3p represented by SEQ ID NO: 151, which are produced from the precursor hsa-mir-4433a represented by SEQ ID NO: 405.

FIG. 2 Left panel: the measured expression level values of hsa-miR-4675 (SEQ ID NO: 1) in sera from healthy subjects (280 people) and ovarian tumor patients (303 people) selected as a training cohort were each plotted on the ordinate. The horizontal line in the panel depicts a threshold (7.75) that was optimized by Fisher's discriminant analysis and discriminated between the two groups. Right panel: the measured expression level values of hsa-miR-4675 (SEQ ID NO: 1) in healthy subjects (120 people) and ovarian tumor patients (131 people) selected as a validation cohort were each plotted on the ordinate. The horizontal line in the panel depicts the threshold (7.75) that was set in the training cohort and discriminated between the two groups.

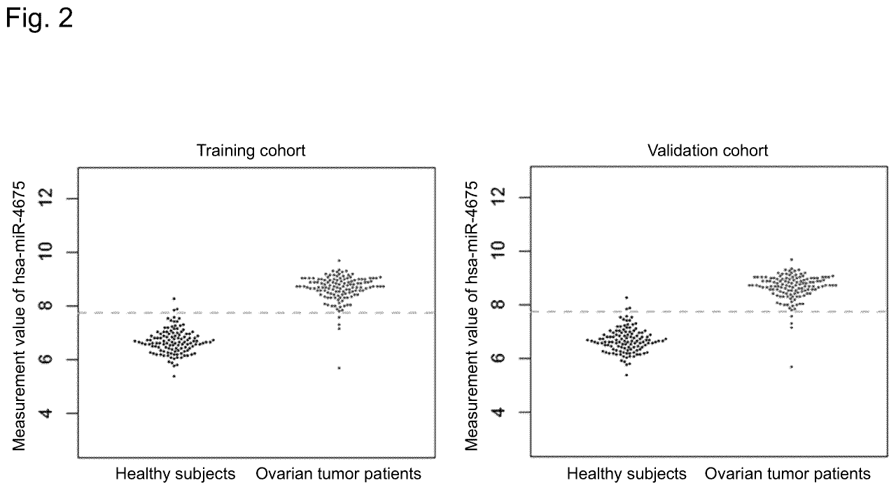

FIG. 3 Upper panel: a discriminant (1.390×hsa-miR-296-5p-1.095×hsa-miR-3131+0.201×hsa-miR-1290+0.695×hsa-miR-642a-3p+0.306×hsa-miR-128-2-5p-13.186) was prepared by use of Fisher's discriminant analysis from the measured expression level values of hsa-miR-296-5p (SEQ ID NO: 256), hsa-miR-3131 (SEQ ID NO: 196), hsa-miR-1290 (SEQ ID NO: 260), hsa-miR-642a-3p (SEQ ID NO: 268), and hsa-miR-128-2-5p (SEQ ID NO: 252) in sera of subjects without ovarian tumor (total 817 people including 280 healthy subjects, 257 benign bone and soft tissue tumor patients and benign breast disease patients, 280 patients having a cancer other than ovarian cancer) and ovarian tumor patients (303 people) selected as a training cohort, and discriminant scores obtained from the discriminant were plotted on the ordinate against the sample groups on the abscissa. The dotted line in the panel depicts a discriminant boundary that offered a discriminant score of 0 and discriminated between the two groups. Lower panel: discriminant scores obtained from assignment to the discriminant (1.390×hsa-miR-296-5p-1.095×hsa-miR-3131+0.201×hsa-miR-1290+0.695×hsa-miR-642a-3p+0.306×hsa-miR-128-2-5p-13.186) prepared from the training cohort as to the expression level measurement values of hsa-miR-296-5p (SEQ ID NO: 256), hsa-miR-3131 (SEQ ID NO: 196), hsa-miR-1290 (SEQ ID NO: 260), hsa-miR-642a-3p (SEQ ID NO: 268), and hsa-miR-128-2-5p (SEQ ID NO: 252) in sera of subjects without ovarian tumor (total 351 people including 120 healthy subjects, 111 benign bone and soft tissue tumor patients and benign breast disease patients, 120 patients having a cancer other than ovarian cancer) and ovarian tumor patients (131 people) selected as a validation cohort were plotted on the ordinate against the sample groups on the abscissa. The dotted line in the panel depicts the discriminant boundary that offered a discriminant score of 0 and discriminated between the two groups.

### MODES FOR CARRYING OUT THE INVENTION

Hereinafter, the present invention will be further described in detail.

### 1. Target Nucleic Acid for Ovarian Tumor

Primary target nucleic acids, as ovarian tumor markers, for detecting the presence and/or absence of ovarian tumor or ovarian tumor cells using the nucleic acids such as the nucleic acid probes or the primers for the detection of ovarian tumor defined above according to the present invention comprise at least one miRNA selected from the group consisting of the following miRNAs: hsa-miR-4675, hsa-miR-4783-3p, hsa-miR-1228-5p, hsa-miR-4532, hsa-miR-6802-5p, hsa-miR-6784-5p, hsa-miR-3940-5p, hsa-miR-1307-3p, hsa-miR-8073, hsa-miR-3184-5p, hsa-miR-1233-5p, hsa-miR-6088, hsa-miR-5195-3p, hsa-miR-320b, hsa-miR-4649-5p, hsa-miR-6800-5p, hsa-miR-1343-3p, hsa-miR-4730, hsa-miR-6885-5p, hsa-miR-5100, hsa-miR-1203, hsa-miR-6756-5p, hsa-miR-373-5p, hsa-miR-1268a, hsa-miR-1260b, hsa-miR-4258, hsa-miR-4697-5p, hsa-miR-1469, hsa-miR-4515, hsa-miR-6861-5p, hsa-miR-6821-5p, hsa-miR-575, hsa-miR-6805-5p, hsa-miR-4758-5p, hsa-miR-3663-3p, hsa-miR-4530, hsa-miR-6798-5p, hsa-miR-6781-5p, hsa-miR-885-3p, hsa-miR-1273g-3p, hsa-miR-4787-3p, hsa-miR-4454, hsa-miR-4706, hsa-miR-1249-3p, hsa-miR-887-3p, hsa-miR-6786-5p, hsa-miR-1238-5p, hsa-miR-6749-5p, hsa-miR-6729-5p, hsa-miR-6825-5p, hsa-miR-663b, hsa-miR-6858-5p, hsa-miR-4690-5p, hsa-miR-6765-5p, hsa-miR-4710, hsa-miR-6775-5p, hsa-miR-371a-5p, hsa-miR-6816-5p, hsa-miR-296-3p, hsa-miR-7977, hsa-miR-8069, hsa-miR-6515-3p, hsa-miR-4687-5p, hsa-miR-1343-5p, hsa-miR-7110-5p, hsa-miR-4525, hsa-miR-3158-5p, hsa-miR-6787-5p, hsa-miR-614, hsa-miR-4689, hsa-miR-1185-2-3p, hsa-miR-1268b, hsa-miR-1228-3p, hsa-miR-1185-1-3p, hsa-miR-940, hsa-miR-939-5p, hsa-miR-6757-5p, hsa-miR-1275, hsa-miR-5001-5p, hsa-miR-6826-5p, hsa-miR-6765-3p, hsa-miR-3679-3p, hsa-miR-4718, hsa-miR-4286, hsa-miR-8059, hsa-miR-4447, hsa-miR-4448, hsa-miR-658, hsa-miR-6766-3p, hsa-miR-197-5p, hsa-miR-6887-5p, hsa-miR-6742-5p, hsa-miR-6729-3p, hsa-miR-5090, hsa-miR-7975, hsa-miR-4505, hsa-miR-6889-5p, hsa-miR-4708-3p, hsa-miR-6131, hsa-miR-1225-3p, hsa-miR-6132, hsa-miR-4734, hsa-miR-3194-3p, hsa-miR-638, hsa-miR-2467-3p, hsa-miR-4728-5p, hsa-miR-5572, hsa-miR-6789-5p, hsa-miR-8063, hsa-miR-4429, hsa-miR-6840-3p, hsa-miR-4476, hsa-miR-675-5p, hsa-miR-711, hsa-miR-6875-5p, hsa-miR-3160-5p, hsa-miR-1908-5p, hsa-miR-6726-5p, hsa-miR-1913, hsa-miR-8071, hsa-miR-3648, hsa-miR-4732-5p, hsa-miR-4787-5p, hsa-miR-3917, hsa-miR-619-5p, hsa-miR-1231, hsa-miR-342-5p, hsa-miR-4433a-5p, hsa-miR-6766-5p, hsa-miR-4707-5p, hsa-miR-7114-5p, hsa-miR-6872-3p, hsa-miR-6780b-5p, hsa-miR-7845-5p, hsa-miR-6798-3p, hsa-miR-665, hsa-miR-6848-5p, hsa-miR-5008-5p, hsa-miR-4294, hsa-miR-6511a-5p, hsa-miR-4435, hsa-miR-4747-3p, hsa-miR-6880-3p, hsa-miR-6869-5p, hsa-miR-7150, hsa-miR-1260a, hsa-miR-6877-5p, hsa-miR-6721-5p, hsa-miR-4656, hsa-miR-1229-5p, hsa-miR-4433a-3p, hsa-miR-4274, hsa-miR-4419b, hsa-miR-4674, hsa-miR-6893-5p, hsa-miR-6763-3p, hsa-miR-6762-5p, hsa-miR-6738-5p, hsa-miR-4513, hsa-miR-6746-5p, hsa-miR-6880-5p, hsa-miR-4736, hsa-miR-718, hsa-miR-6717-5p, hsa-miR-7847-3p, hsa-miR-760, hsa-miR-1199-5p, hsa-miR-6813-5p, hsa-miR-6769a-5p, hsa-miR-1193, hsa-miR-7108-3p, hsa-miR-6741-5p, hsa-miR-4298, hsa-miR-6796-3p, hsa-miR-4750-5p, hsa-miR-6785-5p, hsa-miR-1292-3p, hsa-miR-4749-3p, hsa-miR-6800-3p, hsa-miR-4722-5p, hsa-miR-4746-3p, hsa-miR-4450, hsa-miR-6795-5p, hsa-miR-365a-5p, hsa-miR-498, hsa-miR-6797-5p, hsa-miR-1470, hsa-miR-6851-5p, hsa-miR-1247-3p, hsa-miR-5196-5p, hsa-miR-208a-5p, hsa-miR-6842-5p, hsa-miR-150-3p, hsa-miR-4534, hsa-miR-3135b, hsa-miR-3131, hsa-miR-4792, hsa-miR-6510-5p, hsa-miR-504-3p, hsa-miR-3619-3p, hsa-miR-671-5p, hsa-miR-4667-5p, hsa-miR-4430, hsa-miR-3195, hsa-miR-3679-5p, hsa-miR-6076, hsa-miR-6515-5p, hsa-miR-6820-5p, hsa-miR-4634, hsa-miR-187-5p, hsa-miR-6763-5p, hsa-miR-1908-3p, hsa-miR-1181, hsa-miR-6782-5p, hsa-miR-5010-5p, hsa-miR-6870-5p, hsa-miR-6124, hsa-miR-1249-5p, hsa-miR-6511b-5p, hsa-miR-1254, hsa-miR-4727-3p, hsa-miR-4259, hsa-miR-4771, hsa-miR-3622a-5p, hsa-miR-4480, hsa-miR-4740-5p, hsa-miR-6777-5p, hsa-miR-6794-5p, hsa-miR-4687-3p, hsa-miR-6743-5p, hsa-miR-6771-5p, hsa-miR-3141, hsa-miR-3162-5p, hsa-miR-4271, hsa-miR-1227-5p, hsa-miR-4257, hsa-miR-4270, hsa-miR-4516, hsa-miR-4651, hsa-miR-4725-3p, hsa-miR-6125, hsa-miR-6732-5p, hsa-miR-6791-5p, hsa-miR-6819-5p, hsa-miR-6891-5p, hsa-miR-7108-5p, hsa-miR-7109-5p, hsa-miR-642b-3p, and hsa-miR-642a-3p. Furthermore, at least one miRNA selected from the group consisting of the following other ovarian tumor markers that can be combined with these miRNAs, i.e., hsa-miR-320a, hsa-miR-663a, hsa-miR-328-5p, hsa-miR-128-2-5p, hsa-miR-125a-3p, hsa-miR-191-5p, hsa-miR-92b-5p, hsa-miR-296-5p, hsa-miR-1246, hsa-miR-92a-2-5p, hsa-miR-128-1-5p, hsa-miR-1290, hsa-miR-211-3p, hsa-miR-744-5p, hsa-miR-135a-3p, hsa-miR-451a, hsa-miR-625-3p, hsa-miR-92a-3p, hsa-miR-422a, hsa-miR-483-5p, hsa-miR-652-5p, hsa-miR-24-3p, hsa-miR-23b-3p, hsa-miR-23a-3p, hsa-miR-92b-3p, and hsa-miR-22-3p can also be preferably used as target nucleic acids.

These miRNAs include, for example, a human gene comprising a nucleotide sequence represented by any of SEQ ID NOs: 1 to 275 (i.e., hsa-miR-4675, hsa-miR-4783-3p, hsa-miR-1228-5p, hsa-miR-4532, hsa-miR-6802-5p, hsa-miR-6784-5p, hsa-miR-3940-5p, hsa-miR-1307-3p, hsa-miR-8073, hsa-miR-3184-5p, hsa-miR-1233-5p, hsa-miR-6088, hsa-miR-5195-3p, hsa-miR-320b, hsa-miR-4649-5p, hsa-miR-6800-5p, hsa-miR-1343-3p, hsa-miR-4730, hsa-miR-6885-5p, hsa-miR-5100, hsa-miR-1203, hsa-miR-6756-5p, hsa-miR-373-5p, hsa-miR-1268a, hsa-miR-1260b, hsa-miR-4258, hsa-miR-4697-5p, hsa-miR-1469, hsa-miR-4515, hsa-miR-6861-5p, hsa-miR-6821-5p, hsa-miR-575, hsa-miR-6805-5p, hsa-miR-4758-5p, hsa-miR-3663-3p, hsa-miR-4530, hsa-miR-6798-5p, hsa-miR-6781-5p, hsa-miR-885-3p, hsa-miR-1273g-3p, hsa-miR-4787-3p, hsa-miR-4454, hsa-miR-4706, hsa-miR-1249-3p, hsa-miR-887-3p, hsa-miR-6786-5p, hsa-miR-1238-5p, hsa-miR-6749-5p, hsa-miR-6729-5p, hsa-miR-6825-5p, hsa-miR-663b, hsa-miR-6858-5p, hsa-miR-4690-5p, hsa-miR-6765-5p, hsa-miR-4710, hsa-miR-6775-5p, hsa-miR-371a-5p, hsa-miR-6816-5p, hsa-miR-296-3p, hsa-miR-7977, hsa-miR-8069, hsa-miR-6515-3p, hsa-miR-4687-5p, hsa-miR-1343-5p, hsa-miR-7110-5p, hsa-miR-4525, hsa-miR-3158-5p, hsa-miR-6787-5p, hsa-miR-614, hsa-miR-4689, hsa-miR-1185-2-3p, hsa-miR-1268b, hsa-miR-1228-3p, hsa-miR-1185-1-3p, hsa-miR-940, hsa-miR-939-5p, hsa-miR-6757-5p, hsa-miR-1275, hsa-miR-5001-5p, hsa-miR-6826-5p, hsa-miR-6765-3p, hsa-miR-3679-3p, hsa-miR-4718, hsa-miR-4286, hsa-miR-8059, hsa-miR-4447, hsa-miR-4448, hsa-miR-658, hsa-miR-6766-3p, hsa-miR-197-5p, hsa-miR-6887-5p, hsa-miR-6742-5p, hsa-miR-6729-3p, hsa-miR-5090, hsa-miR-7975, hsa-miR-4505, hsa-miR-6889-5p, hsa-miR-4708-3p, hsa-miR-6131, hsa-miR-1225-3p, hsa-miR-6132, hsa-miR-4734, hsa-miR-3194-3p, hsa-miR-638, hsa-miR-2467-3p, hsa-miR-4728-5p, hsa-miR-5572, hsa-miR-6789-5p, hsa-miR-8063, hsa-miR-4429, hsa-miR-6840-3p, hsa-miR-4476, hsa-miR-675-5p, hsa-miR-711, hsa-miR-6875-5p, hsa-miR-3160-5p, hsa-miR-1908-5p, hsa-miR-6726-5p, hsa-miR-1913, hsa-miR-8071, hsa-miR-3648, hsa-miR-4732-5p, hsa-miR-4787-5p, hsa-miR-3917, hsa-miR-619-5p, hsa-miR-1231, hsa-miR-342-5p, hsa-miR-4433a-5p, hsa-miR-6766-5p, hsa-miR-4707-5p, hsa-miR-7114-5p, hsa-miR-6872-3p, hsa-miR-6780b-5p, hsa-miR-7845-5p, hsa-miR-6798-3p, hsa-miR-665, hsa-miR-6848-5p, hsa-miR-5008-5p, hsa-miR-4294, hsa-miR-6511a-5p, hsa-miR-4435, hsa-miR-4747-3p, hsa-miR-6880-3p, hsa-miR-6869-5p, hsa-miR-7150, hsa-miR-1260a, hsa-miR-6877-5p, hsa-miR-6721-5p, hsa-miR-4656, hsa-miR-1229-5p, hsa-miR-4433a-3p, hsa-miR-4274, hsa-miR-4419b, hsa-miR-4674, hsa-miR-6893-5p, hsa-miR-6763-3p, hsa-miR-6762-5p, hsa-miR-6738-5p, hsa-miR-4513, hsa-miR-6746-5p, hsa-miR-6880-5p, hsa-miR-4736, hsa-miR-718, hsa-miR-6717-5p, hsa-miR-7847-3p, hsa-miR-760, hsa-miR-1199-5p, hsa-miR-6813-5p, hsa-miR-6769a-5p, hsa-miR-1193, hsa-miR-7108-3p, hsa-miR-6741-5p, hsa-miR-4298, hsa-miR-6796-3p, hsa-miR-4750-5p, hsa-miR-6785-5p, hsa-miR-1292-3p, hsa-miR-4749-3p, hsa-miR-6800-3p, hsa-miR-4722-5p, hsa-miR-4746-3p, hsa-miR-4450, hsa-miR-6795-5p, hsa-miR-365a-5p, hsa-miR-498, hsa-miR-6797-5p, hsa-miR-1470, hsa-miR-6851-5p, hsa-miR-1247-3p, hsa-miR-5196-5p, hsa-miR-208a-5p, hsa-miR-6842-5p, hsa-miR-150-3p, hsa-miR-4534, hsa-miR-3135b, hsa-miR-3131, hsa-miR-4792, hsa-miR-6510-5p, hsa-miR-504-3p, hsa-miR-3619-3p, hsa-miR-671-5p, hsa-miR-4667-5p, hsa-miR-4430, hsa-miR-3195, hsa-miR-3679-5p, hsa-miR-6076, hsa-miR-6515-5p, hsa-miR-6820-5p, hsa-miR-4634, hsa-miR-187-5p, hsa-miR-6763-5p, hsa-miR-1908-3p, hsa-miR-1181, hsa-miR-6782-5p, hsa-miR-5010-5p, hsa-miR-6870-5p, hsa-miR-6124, hsa-miR-1249-5p, hsa-miR-6511b-5p, hsa-miR-1254, hsa-miR-4727-3p, hsa-miR-4259, hsa-miR-4771, hsa-miR-3622a-5p, hsa-miR-4480, hsa-miR-4740-5p, hsa-miR-6777-5p, hsa-miR-6794-5p, hsa-miR-4687-3p, hsa-miR-6743-5p, hsa-miR-6771-5p, hsa-miR-3141, hsa-miR-3162-5p, hsa-miR-4271, hsa-miR-1227-5p, hsa-miR-4257, hsa-miR-4270, hsa-miR-4516, hsa-miR-4651, hsa-miR-4725-3p, hsa-miR-6125, hsa-miR-6732-5p, hsa-miR-6791-5p, hsa-miR-6819-5p, hsa-miR-6891-5p, hsa-miR-7108-5p, hsa-miR-642b-3p, hsa-miR-642a-3p, hsa-miR-7109-5p, hsa-miR-320a, hsa-miR-663a, hsa-miR-328-5p, hsa-miR-128-2-5p, hsa-miR-125a-3p, hsa-miR-191-5p, hsa-miR-92b-5p, hsa-miR-296-5p, hsa-miR-1246, hsa-miR-92a-2-5p, hsa-miR-128-1-5p, hsa-miR-1290, hsa-miR-211-3p, hsa-miR-744-5p, hsa-miR-135a-3p, hsa-miR-451a, hsa-miR-625-3p, hsa-miR-92a-3p, hsa-miR-422a, hsa-miR-483-5p, hsa-miR-652-5p, hsa-miR-24-3p, hsa-miR-23b-3p, hsa-miR-23a-3p, hsa-miR-92b-3p and hsa-miR-22-3p respectively), a congener thereof, a transcript thereof, or/and a variant or a derivative thereof. In this context, the gene, the congener, the transcript, the variant, and the derivative are as defined above.

The target nucleic acid is preferably a human gene comprising a nucleotide sequence represented by any of SEQ ID NOs: 1 to 842 or a transcript thereof, more preferably the transcript, i.e., a miRNA or its precursor RNA, pri-miRNA or pre-miRNA.

The first target gene is the hsa-miR-4675 gene, a congener thereof, a transcript thereof, or a variant or a derivative thereof. None of the previously known report shows that change in the expression of the gene or the transcript thereof can serve as a marker for ovarian tumor.

The second target gene is the hsa-miR-4783-3p gene, a congener thereof, a transcript thereof, or a variant or a derivative thereof. None of the previously known report shows that change in the expression of the gene or the transcript thereof can serve as a marker for ovarian tumor.

The third target gene is the hsa-miR-1228-5p gene, a congener thereof, a transcript thereof, or a variant or a derivative thereof. None of the previously known report shows that change in the expression of the gene or the transcript thereof can serve as a marker for ovarian tumor.

The fourth target gene is the hsa-miR-4532 gene, a congener thereof, a transcript thereof, or a variant or a derivative thereof. None of the previously known report shows that change in the expression of the gene or the transcript thereof can serve as a marker for ovarian tumor.

The fifth target gene is the hsa-miR-6802-5p gene, a congener thereof, a transcript thereof, or a variant or a derivative thereof. None of the previously known report shows that change in the expression of the gene or the transcript thereof can serve as a marker for ovarian tumor.

The sixth target gene is the hsa-miR-6784-5p gene, a congener thereof, a transcript thereof, or a variant or a derivative thereof. None of the previously known report shows that change in the expression of the gene or the transcript thereof can serve as a marker for ovarian tumor.

The seventh target gene is the hsa-miR-3940-5p gene, a congener thereof, a transcript thereof, or a variant or a derivative thereof. None of the previously known report shows that change in the expression of the gene or the transcript thereof can serve as a marker for ovarian tumor.

The eighth target gene is the hsa-miR-1307-3p gene, a congener thereof, a transcript thereof, or a variant or a derivative thereof. None of the previously known report shows that change in the expression of the gene or the transcript thereof can serve as a marker for ovarian tumor.

The ninth target gene is the hsa-miR-8073 gene, a congener thereof, a transcript thereof, or a variant or a derivative thereof. None of the previously known report shows that change in the expression of the gene or the transcript thereof can serve as a marker for ovarian tumor.

The 10th target gene is the hsa-miR-3184-5p gene, a congener thereof, a transcript thereof, or a variant or a derivative thereof. None of the previously known report shows that change in the expression of the gene or the transcript thereof can serve as a marker for ovarian tumor.

The 11th target gene is the hsa-miR-1233-5p gene, a congener thereof, a transcript thereof, or a variant or a derivative thereof. None of the previously known report shows that change in the expression of the gene or the transcript thereof can serve as a marker for ovarian tumor.

The 12th target gene is the hsa-miR-6088 gene, a congener thereof, a transcript thereof, or a variant or a derivative thereof. None of the previously known report shows that change in the expression of the gene or the transcript thereof can serve as a marker for ovarian tumor.

The 13th target gene is the hsa-miR-5195-3p gene, a congener thereof, a transcript thereof, or a variant or a derivative thereof. None of the previously known report shows that change in the expression of the gene or the transcript thereof can serve as a marker for ovarian tumor.

The 14th target gene is the hsa-miR-320b gene, a congener thereof, a transcript thereof, or a variant or a derivative thereof. None of the previously known report shows that change in the expression of the gene or the transcript thereof can serve as a marker for ovarian tumor.

The 15th target gene is the hsa-miR-4649-5p gene, a congener thereof, a transcript thereof, or a variant or a derivative thereof. None of the previously known report shows that change in the expression of the gene or the transcript thereof can serve as a marker for ovarian tumor.

The 16th target gene is the hsa-miR-6800-5p gene, a congener thereof, a transcript thereof, or a variant or a derivative thereof. None of the previously known report shows that change in the expression of the gene or the transcript thereof can serve as a marker for ovarian tumor.

The 17th target gene is the hsa-miR-1343-3p gene, a congener thereof, a transcript thereof, or a variant or a derivative thereof. None of the previously known report shows that change in the expression of the gene or the transcript thereof can serve as a marker for ovarian tumor.

The 18th target gene is the hsa-miR-4730 gene, a congener thereof, a transcript thereof, or a variant or a derivative thereof. None of the previously known report shows that change in the expression of the gene or the transcript thereof can serve as a marker for ovarian tumor.

The 19th target gene is the hsa-miR-6885-5p gene, a congener thereof, a transcript thereof, or a variant or a derivative thereof. None of the previously known report shows that change in the expression of the gene or the transcript thereof can serve as a marker for ovarian tumor.

The 20th target gene is the hsa-miR-5100 gene, a congener thereof, a transcript thereof, or a variant or a derivative thereof. None of the previously known report shows that change in the expression of the gene or the transcript thereof can serve as a marker for ovarian tumor.

The 21st target gene is the hsa-miR-1203 gene, a congener thereof, a transcript thereof, or a variant or a derivative thereof. None of the previously known report shows that change in the expression of the gene or the transcript thereof can serve as a marker for ovarian tumor.

The 22nd target gene is the hsa-miR-6756-5p gene, a congener thereof, a transcript thereof, or a variant or a derivative thereof. None of the previously known report shows that change in the expression of the gene or the transcript thereof can serve as a marker for ovarian tumor.

The 23rd target gene is the hsa-miR-373-5p gene, a congener thereof, a transcript thereof, or a variant or a derivative thereof. None of the previously known report shows that change in the expression of the gene or the transcript thereof can serve as a marker for ovarian tumor.

The 24th target gene is the hsa-miR-1268a gene, a congener thereof, a transcript thereof, or a variant or a derivative thereof. None of the previously known report shows that change in the expression of the gene or the transcript thereof can serve as a marker for ovarian tumor.

The 25th target gene is the hsa-miR-1260b gene, a congener thereof, a transcript thereof, or a variant or a derivative thereof. None of the previously known report shows that change in the expression of the gene or the transcript thereof can serve as a marker for ovarian tumor.

The 26th target gene is the hsa-miR-4258 gene, a congener thereof, a transcript thereof, or a variant or a derivative thereof. None of the previously known report shows that change in the expression of the gene or the transcript thereof can serve as a marker for ovarian tumor.

The 27th target gene is the hsa-miR-4697-5p gene, a congener thereof, a transcript thereof, or a variant or a derivative thereof. None of the previously known report shows that change in the expression of the gene or the transcript thereof can serve as a marker for ovarian tumor.

The 28th target gene is the hsa-miR-1469 gene, a congener thereof, a transcript thereof, or a variant or a derivative thereof. None of the previously known report shows that change in the expression of the gene or the transcript thereof can serve as a marker for ovarian tumor.

The 29th target gene is the hsa-miR-4515 gene, a congener thereof, a transcript thereof, or a variant or a derivative thereof. None of the previously known report shows that change in the expression of the gene or the transcript thereof can serve as a marker for ovarian tumor.

The 30th target gene is the hsa-miR-6861-5p gene, a congener thereof, a transcript thereof, or a variant or a derivative thereof. None of the previously known report shows that change in the expression of the gene or the transcript thereof can serve as a marker for ovarian tumor.

The 31st target gene is the hsa-miR-6821-5p gene, a congener thereof, a transcript thereof, or a variant or a derivative thereof. None of the previously known report shows that change in the expression of the gene or the transcript thereof can serve as a marker for ovarian tumor.

The 32nd target gene is the hsa-miR-575 gene, a congener thereof, a transcript thereof, or a variant or a derivative thereof. None of the previously known report shows that change in the expression of the gene or the transcript thereof can serve as a marker for ovarian tumor.

The 33rd target gene is the hsa-miR-6805-5p gene, a congener thereof, a transcript thereof, or a variant or a derivative thereof. None of the previously known report shows that change in the expression of the gene or the transcript thereof can serve as a marker for ovarian tumor.

The 34th target gene is the hsa-miR-4758-5p gene, a congener thereof, a transcript thereof, or a variant or a derivative thereof. None of the previously known report shows that change in the expression of the gene or the transcript thereof can serve as a marker for ovarian tumor.

The 35th target gene is the hsa-miR-3663-3p gene, a congener thereof, a transcript thereof, or a variant or a derivative thereof. None of the previously known report shows that change in the expression of the gene or the transcript thereof can serve as a marker for ovarian tumor.

The 36th target gene is the hsa-miR-4530 gene, a congener thereof, a transcript thereof, or a variant or a derivative thereof. None of the previously known report shows that change in the expression of the gene or the transcript thereof can serve as a marker for ovarian tumor.

The 37th target gene is the hsa-miR-6798-5p gene, a congener thereof, a transcript thereof, or a variant or a derivative thereof. None of the previously known report shows that change in the expression of the gene or the transcript thereof can serve as a marker for ovarian tumor.

The 38th target gene is the hsa-miR-6781-5p gene, a congener thereof, a transcript thereof, or a variant or a derivative thereof. None of the previously known report shows that change in the expression of the gene or the transcript thereof can serve as a marker for ovarian tumor.

The 39th target gene is the hsa-miR-885-3p gene, a congener thereof, a transcript thereof, or a variant or a derivative thereof. None of the previously known report shows that change in the expression of the gene or the transcript thereof can serve as a marker for ovarian tumor.

The 40th target gene is the hsa-miR-1273g-3p gene, a congener thereof, a transcript thereof, or a variant or a derivative thereof. None of the previously known report shows that change in the expression of the gene or the transcript thereof can serve as a marker for ovarian tumor.

The 41st target gene is the hsa-miR-4787-3p gene, a congener thereof, a transcript thereof, or a variant or a derivative thereof. None of the previously known report shows that change in the expression of the gene or the transcript thereof can serve as a marker for ovarian tumor.

The 42nd target gene is the hsa-miR-4454 gene, a congener thereof, a transcript thereof, or a variant or a derivative thereof. None of the previously known report shows that change in the expression of the gene or the transcript thereof can serve as a marker for ovarian tumor.

The 43rd target gene is the hsa-miR-4706 gene, a congener thereof, a transcript thereof, or a variant or a derivative thereof. None of the previously known report shows that change in the expression of the gene or the transcript thereof can serve as a marker for ovarian tumor.

The 44th target gene is the hsa-miR-1249-3p gene, a congener thereof, a transcript thereof, or a variant or a derivative thereof. None of the previously known report shows that change in the expression of the gene or the transcript thereof can serve as a marker for ovarian tumor.

The 45th target gene is the hsa-miR-887-3p gene, a congener thereof, a transcript thereof, or a variant or a derivative thereof. None of the previously known report shows that change in the expression of the gene or the transcript thereof can serve as a marker for ovarian tumor.

The 46th target gene is the hsa-miR-6786-5p gene, a congener thereof, a transcript thereof, or a variant or a derivative thereof. None of the previously known report shows that change in the expression of the gene or the transcript thereof can serve as a marker for ovarian tumor.

The 47th target gene is the hsa-miR-1238-5p gene, a congener thereof, a transcript thereof, or a variant or a derivative thereof. None of the previously known report shows that change in the expression of the gene or the transcript thereof can serve as a marker for ovarian tumor.

The 48th target gene is the hsa-miR-6749-5p gene, a congener thereof, a transcript thereof, or a variant or a derivative thereof. None of the previously known report shows that change in the expression of the gene or the transcript thereof can serve as a marker for ovarian tumor.

The 49th target gene is the hsa-miR-6729-5p gene, a congener thereof, a transcript thereof, or a variant or a derivative thereof. None of the previously known report shows that change in the expression of the gene or the transcript thereof can serve as a marker for ovarian tumor.

The 50th target gene is the hsa-miR-6825-5p gene, a congener thereof, a transcript thereof, or a variant or a derivative thereof. None of the previously known report shows that change in the expression of the gene or the transcript thereof can serve as a marker for ovarian tumor.

The 51st target gene is the hsa-miR-663b gene, a congener thereof, a transcript thereof, or a variant or a derivative thereof. None of the previously known report shows that change in the expression of the gene or the transcript thereof can serve as a marker for ovarian tumor.

The 52nd target gene is the hsa-miR-6858-5p gene, a congener thereof, a transcript thereof, or a variant or a derivative thereof. None of the previously known report shows that change in the expression of the gene or the transcript thereof can serve as a marker for ovarian tumor.

The 53rd target gene is the hsa-miR-4690-5p gene, a congener thereof, a transcript thereof, or a variant or a derivative thereof. None of the previously known report shows that change in the expression of the gene or the transcript thereof can serve as a marker for ovarian tumor.

The 54th target gene is the hsa-miR-6765-5p gene, a congener thereof, a transcript thereof, or a variant or a derivative thereof. None of the previously known report shows that change in the expression of the gene or the transcript thereof can serve as a marker for ovarian tumor.

The 55th target gene is the hsa-miR-4710 gene, a congener thereof, a transcript thereof, or a variant or a derivative thereof. None of the previously known report shows that change in the expression of the gene or the transcript thereof can serve as a marker for ovarian tumor.

The 56th target gene is the hsa-miR-6775-5p gene, a congener thereof, a transcript thereof, or a variant or a derivative thereof. None of the previously known report shows that change in the expression of the gene or the transcript thereof can serve as a marker for ovarian tumor.

The 57th target gene is the hsa-miR-371a-5p gene, a congener thereof, a transcript thereof, or a variant or a derivative thereof. None of the previously known report shows that change in the expression of the gene or the transcript thereof can serve as a marker for ovarian tumor.

The 58th target gene is the hsa-miR-6816-5p gene, a congener thereof, a transcript thereof, or a variant or a derivative thereof. None of the previously known report shows that change in the expression of the gene or the transcript thereof can serve as a marker for ovarian tumor.

The 59th target gene is the hsa-miR-296-3p gene, a congener thereof, a transcript thereof, or a variant or a derivative thereof. None of the previously known report shows that change in the expression of the gene or the transcript thereof can serve as a marker for ovarian tumor.

The 60th target gene is the hsa-miR-7977 gene, a congener thereof, a transcript thereof, or a variant or a derivative thereof. None of the previously known report shows that change in the expression of the gene or the transcript thereof can serve as a marker for ovarian tumor.

The 61st target gene is the hsa-miR-8069 gene, a congener thereof, a transcript thereof, or a variant or a derivative thereof. None of the previously known report shows that change in the expression of the gene or the transcript thereof can serve as a marker for ovarian tumor.

The 62nd target gene is the hsa-miR-6515-3p gene, a congener thereof, a transcript thereof, or a variant or a derivative thereof. None of the previously known report shows that change in the expression of the gene or the transcript thereof can serve as a marker for ovarian tumor.

The 63rd target gene is the hsa-miR-4687-5p gene, a congener thereof, a transcript thereof, or a variant or a derivative thereof. None of the previously known report shows that change in the expression of the gene or the transcript thereof can serve as a marker for ovarian tumor.

The 64th target gene is the hsa-miR-1343-5p gene, a congener thereof, a transcript thereof, or a variant or a derivative thereof. None of the previously known report shows that change in the expression of the gene or the transcript thereof can serve as a marker for ovarian tumor.

The 65th target gene is the hsa-miR-7110-5p gene, a congener thereof, a transcript thereof, or a variant or a derivative thereof. None of the previously known report shows that change in the expression of the gene or the transcript thereof can serve as a marker for ovarian tumor.

The 66th target gene is the hsa-miR-4525 gene, a congener thereof, a transcript thereof, or a variant or a derivative thereof. None of the previously known report shows that change in the expression of the gene or the transcript thereof can serve as a marker for ovarian tumor.

The 67th target gene is the hsa-miR-3158-5p gene, a congener thereof, a transcript thereof, or a variant or a derivative thereof. None of the previously known report shows that change in the expression of the gene or the transcript thereof can serve as a marker for ovarian tumor.

The 68th target gene is the hsa-miR-6787-5p gene, a congener thereof, a transcript thereof, or a variant or a derivative thereof. None of the previously known report shows that change in the expression of the gene or the transcript thereof can serve as a marker for ovarian tumor.

The 69th target gene is the hsa-miR-614 gene, a congener thereof, a transcript thereof, or a variant or a derivative thereof. None of the previously known report shows that change in the expression of the gene or the transcript thereof can serve as a marker for ovarian tumor.

The 70th target gene is the hsa-miR-4689 gene, a congener thereof, a transcript thereof, or a variant or a derivative thereof. None of the previously known report shows that change in the expression of the gene or the transcript thereof can serve as a marker for ovarian tumor.

The 71st target gene is the hsa-miR-1185-2-3p gene, a congener thereof, a transcript thereof, or a variant or a derivative thereof. None of the previously known report shows that change in the expression of the gene or the transcript thereof can serve as a marker for ovarian tumor.

The 72nd target gene is the hsa-miR-1268b gene, a congener thereof, a transcript thereof, or a variant or a derivative thereof. None of the previously known report shows that change in the expression of the gene or the transcript thereof can serve as a marker for ovarian tumor.

The 73rd target gene is the hsa-miR-1228-3p gene, a congener thereof, a transcript thereof, or a variant or a derivative thereof. None of the previously known report shows that change in the expression of the gene or the transcript thereof can serve as a marker for ovarian tumor.

The 74th target gene is the hsa-miR-1185-1-3p gene, a congener thereof, a transcript thereof, or a variant or a derivative thereof. None of the previously known report shows that change in the expression of the gene or the transcript thereof can serve as a marker for ovarian tumor.

The 75th target gene is the hsa-miR-940 gene, a congener thereof, a transcript thereof, or a variant or a derivative thereof. None of the previously known report shows that change in the expression of the gene or the transcript thereof can serve as a marker for ovarian tumor.

The 76th target gene is the hsa-miR-939-5p gene, a congener thereof, a transcript thereof, or a variant or a derivative thereof. None of the previously known report shows that change in the expression of the gene or the transcript thereof can serve as a marker for ovarian tumor.

The 77th target gene is the hsa-miR-6757-5p gene, a congener thereof, a transcript thereof, or a variant or a derivative thereof. None of the previously known report shows that change in the expression of the gene or the transcript thereof can serve as a marker for ovarian tumor.

The 78th target gene is the hsa-miR-1275 gene, a congener thereof, a transcript thereof, or a variant or a derivative thereof. None of the previously known report shows that change in the expression of the gene or the transcript thereof can serve as a marker for ovarian tumor.

The 79th target gene is the hsa-miR-5001-5p gene, a congener thereof, a transcript thereof, or a variant or a derivative thereof. None of the previously known report shows that change in the expression of the gene or the transcript thereof can serve as a marker for ovarian tumor.

The 80th target gene is the hsa-miR-6826-5p gene, a congener thereof, a transcript thereof, or a variant or a derivative thereof. None of the previously known report shows that change in the expression of the gene or the transcript thereof can serve as a marker for ovarian tumor.

The 81st target gene is the hsa-miR-6765-3p gene, a congener thereof, a transcript thereof, or a variant or a derivative thereof. None of the previously known report shows that change in the expression of the gene or the transcript thereof can serve as a marker for ovarian tumor.

The 82nd target gene is the hsa-miR-3679-3p gene, a congener thereof, a transcript thereof, or a variant or a derivative thereof. None of the previously known report shows that change in the expression of the gene or the transcript thereof can serve as a marker for ovarian tumor.

The 83rd target gene is the hsa-miR-4718 gene, a congener thereof, a transcript thereof, or a variant or a derivative thereof. None of the previously known report shows that change in the expression of the gene or the transcript thereof can serve as a marker for ovarian tumor.

The 84th target gene is the hsa-miR-4286 gene, a congener thereof, a transcript thereof, or a variant or a derivative thereof. None of the previously known report shows that change in the expression of the gene or the transcript thereof can serve as a marker for ovarian tumor.

The 85th target gene is the hsa-miR-8059 gene, a congener thereof, a transcript thereof, or a variant or a derivative thereof. None of the previously known report shows that change in the expression of the gene or the transcript thereof can serve as a marker for ovarian tumor.

The 86th target gene is the hsa-miR-4447 gene, a congener thereof, a transcript thereof, or a variant or a derivative thereof. None of the previously known report shows that change in the expression of the gene or the transcript thereof can serve as a marker for ovarian tumor.

The 87th target gene is the hsa-miR-4448 gene, a congener thereof, a transcript thereof, or a variant or a derivative thereof. None of the previously known report shows that change in the expression of the gene or the transcript thereof can serve as a marker for ovarian tumor.

The 88th target gene is the hsa-miR-658 gene, a congener thereof, a transcript thereof, or a variant or a derivative thereof. None of the previously known report shows that change in the expression of the gene or the transcript thereof can serve as a marker for ovarian tumor.

The 89th target gene is the hsa-miR-6766-3p gene, a congener thereof, a transcript thereof, or a variant or a derivative thereof. None of the previously known report shows that change in the expression of the gene or the transcript thereof can serve as a marker for ovarian tumor.

The 90th target gene is the hsa-miR-197-5p gene, a congener thereof, a transcript thereof, or a variant or a derivative thereof. None of the previously known report shows that change in the expression of the gene or the transcript thereof can serve as a marker for ovarian tumor.

The 91st target gene is the hsa-miR-6887-5p gene, a congener thereof, a transcript thereof, or a variant or a derivative thereof. None of the previously known report shows that change in the expression of the gene or the transcript thereof can serve as a marker for ovarian tumor.

The 92nd target gene is the hsa-miR-6742-5p gene, a congener thereof, a transcript thereof, or a variant or a derivative thereof. None of the previously known report shows that change in the expression of the gene or the transcript thereof can serve as a marker for ovarian tumor.

The 93rd target gene is the hsa-miR-6729-3p gene, a congener thereof, a transcript thereof, or a variant or a derivative thereof. None of the previously known report shows that change in the expression of the gene or the transcript thereof can serve as a marker for ovarian tumor.

The 94th target gene is the hsa-miR-5090 gene, a congener thereof, a transcript thereof, or a variant or a derivative thereof. None of the previously known report shows that change in the expression of the gene or the transcript thereof can serve as a marker for ovarian tumor.

The 95th target gene is the hsa-miR-7975 gene, a congener thereof, a transcript thereof, or a variant or a derivative thereof. None of the previously known report shows that change in the expression of the gene or the transcript thereof can serve as a marker for ovarian tumor.

The 96th target gene is the hsa-miR-4505 gene, a congener thereof, a transcript thereof, or a variant or a derivative thereof. None of the previously known report shows that change in the expression of the gene or the transcript thereof can serve as a marker for ovarian tumor.

The 97th target gene is the hsa-miR-6889-5p gene, a congener thereof, a transcript thereof, or a variant or a derivative thereof. None of the previously known report shows that change in the expression of the gene or the transcript thereof can serve as a marker for ovarian tumor.

The 98th target gene is the hsa-miR-4708-3p gene, a congener thereof, a transcript thereof, or a variant or a derivative thereof. None of the previously known report shows that change in the expression of the gene or the transcript thereof can serve as a marker for ovarian tumor.

The 99th target gene is the hsa-miR-6131 gene, a congener thereof, a transcript thereof, or a variant or a derivative thereof. None of the previously known report shows that change in the expression of the gene or the transcript thereof can serve as a marker for ovarian tumor.

The 100th target gene is the hsa-miR-1225-3p gene, a congener thereof, a transcript thereof, or a variant or a derivative thereof. None of the previously known report shows that change in the expression of the gene or the transcript thereof can serve as a marker for ovarian tumor.

The 101st target gene is the hsa-miR-6132 gene, a congener thereof, a transcript thereof, or a variant or a derivative thereof. None of the previously known report shows that change in the expression of the gene or the transcript thereof can serve as a marker for ovarian tumor.

The 102nd target gene is the hsa-miR-4734 gene, a congener thereof, a transcript thereof, or a variant or a derivative thereof. None of the previously known report shows that change in the expression of the gene or the transcript thereof can serve as a marker for ovarian tumor.

The 103rd target gene is the hsa-miR-3194-3p gene, a congener thereof, a transcript thereof, or a variant or a derivative thereof. None of the previously known report shows that change in the expression of the gene or the transcript thereof can serve as a marker for ovarian tumor.

The 104th target gene is the hsa-miR-638 gene, a congener thereof, a transcript thereof, or a variant or a derivative thereof. None of the previously known report shows that change in the expression of the gene or the transcript thereof can serve as a marker for ovarian tumor.

The 105th target gene is the hsa-miR-2467-3p gene, a congener thereof, a transcript thereof, or a variant or a derivative thereof. None of the previously known report shows that change in the expression of the gene or the transcript thereof can serve as a marker for ovarian tumor.

The 106th target gene is the hsa-miR-4728-5p gene, a congener thereof, a transcript thereof, or a variant or a derivative thereof. None of the previously known report shows that change in the expression of the gene or the transcript thereof can serve as a marker for ovarian tumor.

The 107th target gene is the hsa-miR-5572 gene, a congener thereof, a transcript thereof, or a variant or a derivative thereof. None of the previously known report shows that change in the expression of the gene or the transcript thereof can serve as a marker for ovarian tumor.

The 108th target gene is the hsa-miR-6789-5p gene, a congener thereof, a transcript thereof, or a variant or a derivative thereof. None of the previously known report shows that change in the expression of the gene or the transcript thereof can serve as a marker for ovarian tumor.

The 109th target gene is the hsa-miR-8063 gene, a congener thereof, a transcript thereof, or a variant or a derivative thereof. None of the previously known report shows that change in the expression of the gene or the transcript thereof can serve as a marker for ovarian tumor.

The 110th target gene is the hsa-miR-4429 gene, a congener thereof, a transcript thereof, or a variant or a derivative thereof. None of the previously known report shows that change in the expression of the gene or the transcript thereof can serve as a marker for ovarian tumor.

The 111th target gene is the hsa-miR-6840-3p gene, a congener thereof, a transcript thereof, or a variant or a derivative thereof. None of the previously known report shows that change in the expression of the gene or the transcript thereof can serve as a marker for ovarian tumor.

The 112th target gene is the hsa-miR-4476 gene, a congener thereof, a transcript thereof, or a variant or a derivative thereof. None of the previously known report shows that change in the expression of the gene or the transcript thereof can serve as a marker for ovarian tumor.

The 113th target gene is the hsa-miR-675-5p gene, a congener thereof, a transcript thereof, or a variant or a derivative thereof. None of the previously known report shows that change in the expression of the gene or the transcript thereof can serve as a marker for ovarian tumor.

The 114th target gene is the hsa-miR-711 gene, a congener thereof, a transcript thereof, or a variant or a derivative thereof. None of the previously known report shows that change in the expression of the gene or the transcript thereof can serve as a marker for ovarian tumor.

The 115th target gene is the hsa-miR-6875-5p gene, a congener thereof, a transcript thereof, or a variant or a derivative thereof. None of the previously known report shows that change in the expression of the gene or the transcript thereof can serve as a marker for ovarian tumor.

The 116th target gene is the hsa-miR-3160-5p gene, a congener thereof, a transcript thereof, or a variant or a derivative thereof. None of the previously known report shows that change in the expression of the gene or the transcript thereof can serve as a marker for ovarian tumor.

The 117th target gene is the hsa-miR-1908-5p gene, a congener thereof, a transcript thereof, or a variant or a derivative thereof. None of the previously known report shows that change in the expression of the gene or the transcript thereof can serve as a marker for ovarian tumor.

The 118th target gene is the hsa-miR-6726-5p gene, a congener thereof, a transcript thereof, or a variant or a derivative thereof. None of the previously known report shows that change in the expression of the gene or the transcript thereof can serve as a marker for ovarian tumor.

The 119th target gene is the hsa-miR-1913 gene, a congener thereof, a transcript thereof, or a variant or a derivative thereof. None of the previously known report shows that change in the expression of the gene or the transcript thereof can serve as a marker for ovarian tumor.

The 120th target gene is the hsa-miR-8071 gene, a congener thereof, a transcript thereof, or a variant or a derivative thereof. None of the previously known report shows that change in the expression of the gene or the transcript thereof can serve as a marker for ovarian tumor.

The 121st target gene is the hsa-miR-3648 gene, a congener thereof, a transcript thereof, or a variant or a derivative thereof. None of the previously known report shows that change in the expression of the gene or the transcript thereof can serve as a marker for ovarian tumor.

The 122nd target gene is the hsa-miR-4732-5p gene, a congener thereof, a transcript thereof, or a variant or a derivative thereof. None of the previously known report shows that change in the expression of the gene or the transcript thereof can serve as a marker for ovarian tumor.

The 123rd target gene is the hsa-miR-4787-5p gene, a congener thereof, a transcript thereof, or a variant or a derivative thereof. None of the previously known report shows that change in the expression of the gene or the transcript thereof can serve as a marker for ovarian tumor.

The 124th target gene is the hsa-miR-3917 gene, a congener thereof, a transcript thereof, or a variant or a derivative thereof. None of the previously known report shows that change in the expression of the gene or the transcript thereof can serve as a marker for ovarian tumor.

The 125th target gene is the hsa-miR-619-5p gene, a congener thereof, a transcript thereof, or a variant or a derivative thereof. None of the previously known report shows that change in the expression of the gene or the transcript thereof can serve as a marker for ovarian tumor.

The 126th target gene is the hsa-miR-1231 gene, a congener thereof, a transcript thereof, or a variant or a derivative thereof. None of the previously known report shows that change in the expression of the gene or the transcript thereof can serve as a marker for ovarian tumor.

The 127th target gene is the hsa-miR-342-5p gene, a congener thereof, a transcript thereof, or a variant or a derivative thereof. None of the previously known report shows that change in the expression of the gene or the transcript thereof can serve as a marker for ovarian tumor.

The 128th target gene is the hsa-miR-4433a-5p gene, a congener thereof, a transcript thereof, or a variant or a derivative thereof. None of the previously known report shows that change in the expression of the gene or the transcript thereof can serve as a marker for ovarian tumor.

The 129th target gene is the hsa-miR-6766-5p gene, a congener thereof, a transcript thereof, or a variant or a derivative thereof. None of the previously known report shows that change in the expression of the gene or the transcript thereof can serve as a marker for ovarian tumor.

The 130th target gene is the hsa-miR-4707-5p gene, a congener thereof, a transcript thereof, or a variant or a derivative thereof. None of the previously known report shows that change in the expression of the gene or the transcript thereof can serve as a marker for ovarian tumor.

The 131st target gene is the hsa-miR-7114-5p gene, a congener thereof, a transcript thereof, or a variant or a derivative thereof. None of the previously known report shows that change in the expression of the gene or the transcript thereof can serve as a marker for ovarian tumor.

The 132nd target gene is the hsa-miR-6872-3p gene, a congener thereof, a transcript thereof, or a variant or a derivative thereof. None of the previously known report shows that change in the expression of the gene or the transcript thereof can serve as a marker for ovarian tumor.

The 133rd target gene is the hsa-miR-6780b-5p gene, a congener thereof, a transcript thereof, or a variant or a derivative thereof. None of the previously known report shows that change in the expression of the gene or the transcript thereof can serve as a marker for ovarian tumor.

The 134th target gene is the hsa-miR-7845-5p gene, a congener thereof, a transcript thereof, or a variant or a derivative thereof. None of the previously known report shows that change in the expression of the gene or the transcript thereof can serve as a marker for ovarian tumor.

The 135th target gene is the hsa-miR-6798-3p gene, a congener thereof, a transcript thereof, or a variant or a derivative thereof. None of the previously known report shows that change in the expression of the gene or the transcript thereof can serve as a marker for ovarian tumor.

The 136th target gene is the hsa-miR-665 gene, a congener thereof, a transcript thereof, or a variant or a derivative thereof. None of the previously known report shows that change in the expression of the gene or the transcript thereof can serve as a marker for ovarian tumor.

The 137th target gene is the hsa-miR-6848-5p gene, a congener thereof, a transcript thereof, or a variant or a derivative thereof. None of the previously known report shows that change in the expression of the gene or the transcript thereof can serve as a marker for ovarian tumor.

The 138th target gene is the hsa-miR-5008-5p gene, a congener thereof, a transcript thereof, or a variant or a derivative thereof. None of the previously known report shows that change in the expression of the gene or the transcript thereof can serve as a marker for ovarian tumor.

The 139th target gene is the hsa-miR-4294 gene, a congener thereof, a transcript thereof, or a variant or a derivative thereof. None of the previously known report shows that change in the expression of the gene or the transcript thereof can serve as a marker for ovarian tumor.

The 140th target gene is the hsa-miR-6511a-5p gene, a congener thereof, a transcript thereof, or a variant or a derivative thereof. None of the previously known report shows that change in the expression of the gene or the transcript thereof can serve as a marker for ovarian tumor.

The 141st target gene is the hsa-miR-4435 gene, a congener thereof, a transcript thereof, or a variant or a derivative thereof. None of the previously known report shows that change in the expression of the gene or the transcript thereof can serve as a marker for ovarian tumor.

The 142nd target gene is the hsa-miR-4747-3p gene, a congener thereof, a transcript thereof, or a variant or a derivative thereof. None of the previously known report shows that change in the expression of the gene or the transcript thereof can serve as a marker for ovarian tumor.

The 143rd target gene is the hsa-miR-6880-3p gene, a congener thereof, a transcript thereof, or a variant or a derivative thereof. None of the previously known report shows that change in the expression of the gene or the transcript thereof can serve as a marker for ovarian tumor.

The 144th target gene is the hsa-miR-6869-5p gene, a congener thereof, a transcript thereof, or a variant or a derivative thereof. None of the previously known report shows that change in the expression of the gene or the transcript thereof can serve as a marker for ovarian tumor.

The 145th target gene is the hsa-miR-7150 gene, a congener thereof, a transcript thereof, or a variant or a derivative thereof. None of the previously known report shows that change in the expression of the gene or the transcript thereof can serve as a marker for ovarian tumor.

The 146th target gene is the hsa-miR-1260a gene, a congener thereof, a transcript thereof, or a variant or a derivative thereof. None of the previously known report shows that change in the expression of the gene or the transcript thereof can serve as a marker for ovarian tumor.

The 147th target gene is the hsa-miR-6877-5p gene, a congener thereof, a transcript thereof, or a variant or a derivative thereof. None of the previously known report shows that change in the expression of the gene or the transcript thereof can serve as a marker for ovarian tumor.

The 148th target gene is the hsa-miR-6721-5p gene, a congener thereof, a transcript thereof, or a variant or a derivative thereof. None of the previously known report shows that change in the expression of the gene or the transcript thereof can serve as a marker for ovarian tumor.

The 149th target gene is the hsa-miR-4656 gene, a congener thereof, a transcript thereof, or a variant or a derivative thereof. None of the previously known report shows that change in the expression of the gene or the transcript thereof can serve as a marker for ovarian tumor.

The 150th target gene is the hsa-miR-1229-5p gene, a congener thereof, a transcript thereof, or a variant or a derivative thereof. None of the previously known report shows that change in the expression of the gene or the transcript thereof can serve as a marker for ovarian tumor.

The 151st target gene is the hsa-miR-4433a-3p gene, a congener thereof, a transcript thereof, or a variant or a derivative thereof. None of the previously known report shows that change in the expression of the gene or the transcript thereof can serve as a marker for ovarian tumor.

The 152nd target gene is the hsa-miR-4274 gene, a congener thereof, a transcript thereof, or a variant or a derivative thereof. None of the previously known report shows that change in the expression of the gene or the transcript thereof can serve as a marker for ovarian tumor.

The 153rd target gene is the hsa-miR-4419b gene, a congener thereof, a transcript thereof, or a variant or a derivative thereof. None of the previously known report shows that change in the expression of the gene or the transcript thereof can serve as a marker for ovarian tumor.

The 154th target gene is the hsa-miR-4674 gene, a congener thereof, a transcript thereof, or a variant or a derivative thereof. None of the previously known report shows that change in the expression of the gene or the transcript thereof can serve as a marker for ovarian tumor.

The 155th target gene is the hsa-miR-6893-5p gene, a congener thereof, a transcript thereof, or a variant or a derivative thereof. None of the previously known report shows that change in the expression of the gene or the transcript thereof can serve as a marker for ovarian tumor.

The 156th target gene is the hsa-miR-6763-3p gene, a congener thereof, a transcript thereof, or a variant or a derivative thereof. None of the previously known report shows that change in the expression of the gene or the transcript thereof can serve as a marker for ovarian tumor.

The 157th target gene is the hsa-miR-6762-5p gene, a congener thereof, a transcript thereof, or a variant or a derivative thereof. None of the previously known report shows that change in the expression of the gene or the transcript thereof can serve as a marker for ovarian tumor.

The 158th target gene is the hsa-miR-6738-5p gene, a congener thereof, a transcript thereof, or a variant or a derivative thereof. None of the previously known report shows that change in the expression of the gene or the transcript thereof can serve as a marker for ovarian tumor.

The 159th target gene is the hsa-miR-4513 gene, a congener thereof, a transcript thereof, or a variant or a derivative thereof. None of the previously known report shows that change in the expression of the gene or the transcript thereof can serve as a marker for ovarian tumor.

The 160th target gene is the hsa-miR-6746-5p gene, a congener thereof, a transcript thereof, or a variant or a derivative thereof. None of the previously known report shows that change in the expression of the gene or the transcript thereof can serve as a marker for ovarian tumor.

The 161st target gene is the hsa-miR-6880-5p gene, a congener thereof, a transcript thereof, or a variant or a derivative thereof. None of the previously known report shows that change in the expression of the gene or the transcript thereof can serve as a marker for ovarian tumor.

The 162nd target gene is the hsa-miR-4736 gene, a congener thereof, a transcript thereof, or a variant or a derivative thereof. None of the previously known report shows that change in the expression of the gene or the transcript thereof can serve as a marker for ovarian tumor.

The 163rd target gene is the hsa-miR-718 gene, a congener thereof, a transcript thereof, or a variant or a derivative thereof. None of the previously known report shows that change in the expression of the gene or the transcript thereof can serve as a marker for ovarian tumor.

The 164th target gene is the hsa-miR-6717-5p gene, a congener thereof, a transcript thereof, or a variant or a derivative thereof. None of the previously known report shows that change in the expression of the gene or the transcript thereof can serve as a marker for ovarian tumor.

The 165th target gene is the hsa-miR-7847-3p gene, a congener thereof, a transcript thereof, or a variant or a derivative thereof. None of the previously known report shows that change in the expression of the gene or the transcript thereof can serve as a marker for ovarian tumor.

The 166th target gene is the hsa-miR-760 gene, a congener thereof, a transcript thereof, or a variant or a derivative thereof. None of the previously known report shows that change in the expression of the gene or the transcript thereof can serve as a marker for ovarian tumor.

The 167th target gene is the hsa-miR-1199-5p gene, a congener thereof, a transcript thereof, or a variant or a derivative thereof. None of the previously known report shows that change in the expression of the gene or the transcript thereof can serve as a marker for ovarian tumor.

The 168th target gene is the hsa-miR-6813-5p gene, a congener thereof, a transcript thereof, or a variant or a derivative thereof. None of the previously known report shows that change in the expression of the gene or the transcript thereof can serve as a marker for ovarian tumor.

The 169th target gene is the hsa-miR-6769a-5p gene, a congener thereof, a transcript thereof, or a variant or a derivative thereof. None of the previously known report shows that change in the expression of the gene or the transcript thereof can serve as a marker for ovarian tumor.

The 170th target gene is the hsa-miR-1193 gene, a congener thereof, a transcript thereof, or a variant or a derivative thereof. None of the previously known report shows that change in the expression of the gene or the transcript thereof can serve as a marker for ovarian tumor.

The 171st target gene is the hsa-miR-7108-3p gene, a congener thereof, a transcript thereof, or a variant or a derivative thereof. None of the previously known report shows that change in the expression of the gene or the transcript thereof can serve as a marker for ovarian tumor.

The 172nd target gene is the hsa-miR-6741-5p gene, a congener thereof, a transcript thereof, or a variant or a derivative thereof. None of the previously known report shows that change in the expression of the gene or the transcript thereof can serve as a marker for ovarian tumor.

The 173rd target gene is the hsa-miR-4298 gene, a congener thereof, a transcript thereof, or a variant or a derivative thereof. None of the previously known report shows that change in the expression of the gene or the transcript thereof can serve as a marker for ovarian tumor.

The 174th target gene is the hsa-miR-6796-3p gene, a congener thereof, a transcript thereof, or a variant or a derivative thereof. None of the previously known report shows that change in the expression of the gene or the transcript thereof can serve as a marker for ovarian tumor.

The 175th target gene is the hsa-miR-4750-5p gene, a congener thereof, a transcript thereof, or a variant or a derivative thereof. None of the previously known report shows that change in the expression of the gene or the transcript thereof can serve as a marker for ovarian tumor.

The 176th target gene is the hsa-miR-6785-5p gene, a congener thereof, a transcript thereof, or a variant or a derivative thereof. None of the previously known report shows that change in the expression of the gene or the transcript thereof can serve as a marker for ovarian tumor.

The 177th target gene is the hsa-miR-1292-3p gene, a congener thereof, a transcript thereof, or a variant or a derivative thereof. None of the previously known report shows that change in the expression of the gene or the transcript thereof can serve as a marker for ovarian tumor.

The 178th target gene is the hsa-miR-4749-3p gene, a congener thereof, a transcript thereof, or a variant or a derivative thereof. None of the previously known report shows that change in the expression of the gene or the transcript thereof can serve as a marker for ovarian tumor.

The 179th target gene is the hsa-miR-6800-3p gene, a congener thereof, a transcript thereof, or a variant or a derivative thereof. None of the previously known report shows that change in the expression of the gene or the transcript thereof can serve as a marker for ovarian tumor.

The 180th target gene is the hsa-miR-4722-5p gene, a congener thereof, a transcript thereof, or a variant or a derivative thereof. None of the previously known report shows that change in the expression of the gene or the transcript thereof can serve as a marker for ovarian tumor.

The 181st target gene is the hsa-miR-4746-3p gene, a congener thereof, a transcript thereof, or a variant or a derivative thereof. None of the previously known report shows that change in the expression of the gene or the transcript thereof can serve as a marker for ovarian tumor.

The 182nd target gene is the hsa-miR-4450 gene, a congener thereof, a transcript thereof, or a variant or a derivative thereof. None of the previously known report shows that change in the expression of the gene or the transcript thereof can serve as a marker for ovarian tumor.

The 183rd target gene is the hsa-miR-6795-5p gene, a congener thereof, a transcript thereof, or a variant or a derivative thereof. None of the previously known report shows that change in the expression of the gene or the transcript thereof can serve as a marker for ovarian tumor.

The 184th target gene is the hsa-miR-365a-5p gene, a congener thereof, a transcript thereof, or a variant or a derivative thereof. None of the previously known report shows that change in the expression of the gene or the transcript thereof can serve as a marker for ovarian tumor.

The 185th target gene is the hsa-miR-498 gene, a congener thereof, a transcript thereof, or a variant or a derivative thereof. None of the previously known report shows that change in the expression of the gene or the transcript thereof can serve as a marker for ovarian tumor.

The 186th target gene is the hsa-miR-6797-5p gene, a congener thereof, a transcript thereof, or a variant or a derivative thereof. None of the previously known report shows that change in the expression of the gene or the transcript thereof can serve as a marker for ovarian tumor.

The 187th target gene is the hsa-miR-1470 gene, a congener thereof, a transcript thereof, or a variant or a derivative thereof. None of the previously known report shows that change in the expression of the gene or the transcript thereof can serve as a marker for ovarian tumor.

The 188th target gene is the hsa-miR-6851-5p gene, a congener thereof, a transcript thereof, or a variant or a derivative thereof. None of the previously known report shows that change in the expression of the gene or the transcript thereof can serve as a marker for ovarian tumor.

The 189th target gene is the hsa-miR-1247-3p gene, a congener thereof, a transcript thereof, or a variant or a derivative thereof. None of the previously known report shows that change in the expression of the gene or the transcript thereof can serve as a marker for ovarian tumor.

The 190th target gene is the hsa-miR-5196-5p gene, a congener thereof, a transcript thereof, or a variant or a derivative thereof. None of the previously known report shows that change in the expression of the gene or the transcript thereof can serve as a marker for ovarian tumor.

The 191st target gene is the hsa-miR-208a-5p gene, a congener thereof, a transcript thereof, or a variant or a derivative thereof. None of the previously known report shows that change in the expression of the gene or the transcript thereof can serve as a marker for ovarian tumor.

The 192nd target gene is the hsa-miR-6842-5p gene, a congener thereof, a transcript thereof, or a variant or a derivative thereof. None of the previously known report shows that change in the expression of the gene or the transcript thereof can serve as a marker for ovarian tumor.

The 193rd target gene is the hsa-miR-150-3p gene, a congener thereof, a transcript thereof, or a variant or a derivative thereof. None of the previously known report shows that change in the expression of the gene or the transcript thereof can serve as a marker for ovarian tumor.

The 194th target gene is the hsa-miR-4534 gene, a congener thereof, a transcript thereof, or a variant or a derivative thereof. None of the previously known report shows that change in the expression of the gene or the transcript thereof can serve as a marker for ovarian tumor.

The 195th target gene is the hsa-miR-3135b gene, a congener thereof, a transcript thereof, or a variant or a derivative thereof. None of the previously known report shows that change in the expression of the gene or the transcript thereof can serve as a marker for ovarian tumor.

The 196th target gene is the hsa-miR-3131 gene, a congener thereof, a transcript thereof, or a variant or a derivative thereof. None of the previously known report shows that change in the expression of the gene or the transcript thereof can serve as a marker for ovarian tumor.

The 197th target gene is the hsa-miR-4792 gene, a congener thereof, a transcript thereof, or a variant or a derivative thereof. None of the previously known report shows that change in the expression of the gene or the transcript thereof can serve as a marker for ovarian tumor.

The 198th target gene is the hsa-miR-6510-5p gene, a congener thereof, a transcript thereof, or a variant or a derivative thereof. None of the previously known report shows that change in the expression of the gene or the transcript thereof can serve as a marker for ovarian tumor.

The 199th target gene is the hsa-miR-504-3p gene, a congener thereof, a transcript thereof, or a variant or a derivative thereof. None of the previously known report shows that change in the expression of the gene or the transcript thereof can serve as a marker for ovarian tumor.

The 200th target gene is the hsa-miR-3619-3p gene, a congener thereof, a transcript thereof, or a variant or a derivative thereof. None of the previously known report shows that change in the expression of the gene or the transcript thereof can serve as a marker for ovarian tumor.

The 201st target gene is the hsa-miR-671-5p gene, a congener thereof, a transcript thereof, or a variant or a derivative thereof. None of the previously known report shows that change in the expression of the gene or the transcript thereof can serve as a marker for ovarian tumor.

The 202nd target gene is the hsa-miR-4667-5p gene, a congener thereof, a transcript thereof, or a variant or a derivative thereof. None of the previously known report shows that change in the expression of the gene or the transcript thereof can serve as a marker for ovarian tumor.

The 203rd target gene is the hsa-miR-4430 gene, a congener thereof, a transcript thereof, or a variant or a derivative thereof. None of the previously known report shows that change in the expression of the gene or the transcript thereof can serve as a marker for ovarian tumor.

The 204th target gene is the hsa-miR-3195 gene, a congener thereof, a transcript thereof, or a variant or a derivative thereof. None of the previously known report shows that change in the expression of the gene or the transcript thereof can serve as a marker for ovarian tumor.

The 205th target gene is the hsa-miR-3679-5p gene, a congener thereof, a transcript thereof, or a variant or a derivative thereof. None of the previously known report shows that change in the expression of the gene or the transcript thereof can serve as a marker for ovarian tumor.

The 206th target gene is the hsa-miR-6076 gene, a congener thereof, a transcript thereof, or a variant or a derivative thereof. None of the previously known report shows that change in the expression of the gene or the transcript thereof can serve as a marker for ovarian tumor.

The 207th target gene is the hsa-miR-6515-5p gene, a congener thereof, a transcript thereof, or a variant or a derivative thereof. None of the previously known report shows that change in the expression of the gene or the transcript thereof can serve as a marker for ovarian tumor.

The 208th target gene is the hsa-miR-6820-5p gene, a congener thereof, a transcript thereof, or a variant or a derivative thereof. None of the previously known report shows that change in the expression of the gene or the transcript thereof can serve as a marker for ovarian tumor.

The 209th target gene is the hsa-miR-4634 gene, a congener thereof, a transcript thereof, or a variant or a derivative thereof. None of the previously known report shows that change in the expression of the gene or the transcript thereof can serve as a marker for ovarian tumor.

The 210th target gene is the hsa-miR-187-5p gene, a congener thereof, a transcript thereof, or a variant or a derivative thereof. None of the previously known report shows that change in the expression of the gene or the transcript thereof can serve as a marker for ovarian tumor.

The 211th target gene is the hsa-miR-6763-5p gene, a congener thereof, a transcript thereof, or a variant or a derivative thereof. None of the previously known report shows that change in the expression of the gene or the transcript thereof can serve as a marker for ovarian tumor.

The 212th target gene is the hsa-miR-1908-3p gene, a congener thereof, a transcript thereof, or a variant or a derivative thereof. None of the previously known report shows that change in the expression of the gene or the transcript thereof can serve as a marker for ovarian tumor.

The 213th target gene is the hsa-miR-1181 gene, a congener thereof, a transcript thereof, or a variant or a derivative thereof. None of the previously known report shows that change in the expression of the gene or the transcript thereof can serve as a marker for ovarian tumor.

The 214th target gene is the hsa-miR-6782-5p gene, a congener thereof, a transcript thereof, or a variant or a derivative thereof. None of the previously known report shows that change in the expression of the gene or the transcript thereof can serve as a marker for ovarian tumor.

The 215th target gene is the hsa-miR-5010-5p gene, a congener thereof, a transcript thereof, or a variant or a derivative thereof. None of the previously known report shows that change in the expression of the gene or the transcript thereof can serve as a marker for ovarian tumor.

The 216th target gene is the hsa-miR-6870-5p gene, a congener thereof, a transcript thereof, or a variant or a derivative thereof. None of the previously known report shows that change in the expression of the gene or the transcript thereof can serve as a marker for ovarian tumor.

The 217th target gene is the hsa-miR-6124 gene, a congener thereof, a transcript thereof, or a variant or a derivative thereof. None of the previously known report shows that change in the expression of the gene or the transcript thereof can serve as a marker for ovarian tumor.

The 218th target gene is the hsa-miR-1249-5p gene, a congener thereof, a transcript thereof, or a variant or a derivative thereof. None of the previously known report shows that change in the expression of the gene or the transcript thereof can serve as a marker for ovarian tumor.

The 219th target gene is the hsa-miR-6511b-5p gene, a congener thereof, a transcript thereof, or a variant or a derivative thereof. None of the previously known report shows that change in the expression of the gene or the transcript thereof can serve as a marker for ovarian tumor.

The 220th target gene is the hsa-miR-1254 gene, a congener thereof, a transcript thereof, or a variant or a derivative thereof. None of the previously known report shows that change in the expression of the gene or the transcript thereof can serve as a marker for ovarian tumor.

The 221st target gene is the hsa-miR-4727-3p gene, a congener thereof, a transcript thereof, or a variant or a derivative thereof. None of the previously known report shows that change in the expression of the gene or the transcript thereof can serve as a marker for ovarian tumor.

The 222nd target gene is the hsa-miR-4259 gene, a congener thereof, a transcript thereof, or a variant or a derivative thereof. None of the previously known report shows that change in the expression of the gene or the transcript thereof can serve as a marker for ovarian tumor.

The 223rd target gene is the hsa-miR-4771 gene, a congener thereof, a transcript thereof, or a variant or a derivative thereof. None of the previously known report shows that change in the expression of the gene or the transcript thereof can serve as a marker for ovarian tumor.

The 224th target gene is the hsa-miR-3622a-5p gene, a congener thereof, a transcript thereof, or a variant or a derivative thereof. None of the previously known report shows that change in the expression of the gene or the transcript thereof can serve as a marker for ovarian tumor.

The 225th target gene is the hsa-miR-4480 gene, a congener thereof, a transcript thereof, or a variant or a derivative thereof. None of the previously known report shows that change in the expression of the gene or the transcript thereof can serve as a marker for ovarian tumor.

The 226th target gene is the hsa-miR-4740-5p gene, a congener thereof, a transcript thereof, or a variant or a derivative thereof. None of the previously known report shows that change in the expression of the gene or the transcript thereof can serve as a marker for ovarian tumor.

The 227th target gene is the hsa-miR-6777-5p gene, a congener thereof, a transcript thereof, or a variant or a derivative thereof. None of the previously known report shows that change in the expression of the gene or the transcript thereof can serve as a marker for ovarian tumor.

The 228th target gene is the hsa-miR-6794-5p gene, a congener thereof, a transcript thereof, or a variant or a derivative thereof. None of the previously known report shows that change in the expression of the gene or the transcript thereof can serve as a marker for ovarian tumor.

The 229th target gene is the hsa-miR-4687-3p gene, a congener thereof, a transcript thereof, or a variant or a derivative thereof. None of the previously known report shows that change in the expression of the gene or the transcript thereof can serve as a marker for ovarian tumor.

The 230th target gene is the hsa-miR-6743-5p gene, a congener thereof, a transcript thereof, or a variant or a derivative thereof. None of the previously known report shows that change in the expression of the gene or the transcript thereof can serve as a marker for ovarian tumor.

The 231st target gene is the hsa-miR-6771-5p gene, a congener thereof, a transcript thereof, or a variant or a derivative thereof. None of the previously known report shows that change in the expression of the gene or the transcript thereof can serve as a marker for ovarian tumor.

The 232nd target gene is the hsa-miR-3141 gene, a congener thereof, a transcript thereof, or a variant or a derivative thereof. None of the previously known report shows that change in the expression of the gene or the transcript thereof can serve as a marker for ovarian tumor.

The 233rd target gene is the hsa-miR-3162-5p gene, a congener thereof, a transcript thereof, or a variant or a derivative thereof. None of the previously known report shows that change in the expression of the gene or the transcript thereof can serve as a marker for ovarian tumor.

The 234th target gene is the hsa-miR-4271 gene, a congener thereof, a transcript thereof, or a variant or a derivative thereof. None of the previously known report shows that change in the expression of the gene or the transcript thereof can serve as a marker for ovarian tumor.

The 235th target gene is the hsa-miR-1227-5p gene, a congener thereof, a transcript thereof, or a variant or a derivative thereof. None of the previously known report shows that change in the expression of the gene or the transcript thereof can serve as a marker for ovarian tumor.

The 236th target gene is the hsa-miR-4257 gene, a congener thereof, a transcript thereof, or a variant or a derivative thereof. None of the previously known report shows that change in the expression of the gene or the transcript thereof can serve as a marker for ovarian tumor.

The 237th target gene is the hsa-miR-4270 gene, a congener thereof, a transcript thereof, or a variant or a derivative thereof. None of the previously known report shows that change in the expression of the gene or the transcript thereof can serve as a marker for ovarian tumor.

The 238th target gene is the hsa-miR-4516 gene, a congener thereof, a transcript thereof, or a variant or a derivative thereof. None of the previously known report shows that change in the expression of the gene or the transcript thereof can serve as a marker for ovarian tumor.

The 239th target gene is the hsa-miR-4651 gene, a congener thereof, a transcript thereof, or a variant or a derivative thereof. None of the previously known report shows that change in the expression of the gene or the transcript thereof can serve as a marker for ovarian tumor.

The 240th target gene is the hsa-miR-4725-3p gene, a congener thereof, a transcript thereof, or a variant or a derivative thereof. None of the previously known report shows that change in the expression of the gene or the transcript thereof can serve as a marker for ovarian tumor.

The 241st target gene is the hsa-miR-6125 gene, a congener thereof, a transcript thereof, or a variant or a derivative thereof. None of the previously known report shows that change in the expression of the gene or the transcript thereof can serve as a marker for ovarian tumor.

The 242nd target gene is the hsa-miR-6732-5p gene, a congener thereof, a transcript thereof, or a variant or a derivative thereof. None of the previously known report shows that change in the expression of the gene or the transcript thereof can serve as a marker for ovarian tumor.

The 243rd target gene is the hsa-miR-6791-5p gene, a congener thereof, a transcript thereof, or a variant or a derivative thereof. None of the previously known report shows that change in the expression of the gene or the transcript thereof can serve as a marker for ovarian tumor.

The 244th target gene is the hsa-miR-6819-5p gene, a congener thereof, a transcript thereof, or a variant or a derivative thereof. None of the previously known report shows that change in the expression of the gene or the transcript thereof can serve as a marker for ovarian tumor.

The 245th target gene is the hsa-miR-6891-5p gene, a congener thereof, a transcript thereof, or a variant or a derivative thereof. None of the previously known report shows that change in the expression of the gene or the transcript thereof can serve as a marker for ovarian tumor.

The 246th target gene is the hsa-miR-7108-5p gene, a congener thereof, a transcript thereof, or a variant or a derivative thereof. None of the previously known report shows that change in the expression of the gene or the transcript thereof can serve as a marker for ovarian tumor.

The 247th target gene is the hsa-miR-7109-5p gene, a congener thereof, a transcript thereof, or a variant or a derivative thereof. None of the previously known report shows that change in the expression of the gene or the transcript thereof can serve as a marker for ovarian tumor.

The 248th target gene is the hsa-miR-320a gene, a congener thereof, a transcript thereof, or a variant or a derivative thereof. The previously known report shows that change in the expression of the gene or the transcript thereof can serve as a marker for ovarian tumor (Patent Literature 1).

The 249th target gene is the hsa-miR-663a gene, a congener thereof, a transcript thereof, or a variant or a derivative thereof. The previously known report shows that change in the expression of the gene or the transcript thereof can serve as a marker for ovarian tumor (Non-Patent Literature 3).

The 250th target gene is the hsa-miR-328-5p gene, a congener thereof, a transcript thereof, or a variant or a derivative thereof. The previously known report shows that change in the expression of the gene or the transcript thereof can serve as a marker for ovarian tumor (Patent Literature 1).

The 251st target gene is the hsa-miR-642b-3p gene, a congener thereof, a transcript thereof, or a variant or a derivative thereof. None of the previously known report shows that change in the expression of the gene or the transcript thereof can serve as a marker for ovarian tumor.

The 252nd target gene is the hsa-miR-128-2-5p gene, a congener thereof, a transcript thereof, or a variant or a derivative thereof. The previously known report shows that change in the expression of the gene or the transcript thereof can serve as a marker for ovarian tumor (Non-Patent Literature 1).

The 253rd target gene is the hsa-miR-125a-3p gene, a congener thereof, a transcript thereof, or a variant or a derivative thereof. The previously known report shows that change in the expression of the gene or the transcript thereof can serve as a marker for ovarian tumor (Patent Literature 3).

The 254th target gene is the hsa-miR-191-5p gene, a congener thereof, a transcript thereof, or a variant or a derivative thereof. The previously known report shows that change in the expression of the gene or the transcript thereof can serve as a marker for ovarian tumor (Patent Literature 1).

The 255th target gene is the hsa-miR-92b-5p gene, a congener thereof, a transcript thereof, or a variant or a derivative thereof. The previously known report shows that change in the expression of the gene or the transcript thereof can serve as a marker for ovarian tumor (Non-Patent Literature 3).

The 256th target gene is the hsa-miR-296-5p gene, a congener thereof, a transcript thereof, or a variant or a derivative thereof. The previously known report shows that change in the expression of the gene or the transcript thereof can serve as a marker for ovarian tumor (Non-Patent Literature 3).

The 257th target gene is the hsa-miR-1246 gene, a congener thereof, a transcript thereof, or a variant or a derivative thereof. The previously known report shows that change in the expression of the gene or the transcript thereof can serve as a marker for ovarian tumor (Patent Literature 4).

The 258th target gene is the hsa-miR-92a-2-5p gene, a congener thereof, a transcript thereof, or a variant or a derivative thereof. The previously known report shows that change in the expression of the gene or the transcript thereof can serve as a marker for ovarian tumor (Non-Patent Literature 1).

The 259th target gene is the hsa-miR-128-1-5p gene, a congener thereof, a transcript thereof, or a variant or a derivative thereof. The previously known report shows that change in the expression of the gene or the transcript thereof can serve as a marker for ovarian tumor (Non-Patent Literature 1).

The 260th target gene is the hsa-miR-1290 gene, a congener thereof, a transcript thereof, or a variant or a derivative thereof. The previously known report shows that change in the expression of the gene or the transcript thereof can serve as a marker for ovarian tumor (Patent Literature 1).

The 261st target gene is the hsa-miR-211-3p gene, a congener thereof, a transcript thereof, or a variant or a derivative thereof. The previously known report shows that change in the expression of the gene or the transcript thereof can serve as a marker for ovarian tumor (Patent Literature 3).

The 262nd target gene is the hsa-miR-744-5p gene, a congener thereof, a transcript thereof, or a variant or a derivative thereof. The previously known report shows that change in the expression of the gene or the transcript thereof can serve as a marker for ovarian tumor (Patent Literature 4).

The 263rd target gene is the hsa-miR-135a-3p gene, a congener thereof, a transcript thereof, or a variant or a derivative thereof. The previously known report shows that change in the expression of the gene or the transcript thereof can serve as a marker for ovarian tumor (Patent Literature 2).

The 264th target gene is the hsa-miR-451a gene, a congener thereof, a transcript thereof, or a variant or a derivative thereof. The previously known report shows that change in the expression of the gene or the transcript thereof can serve as a marker for ovarian tumor (Non-Patent Literature 2).

The 265th target gene is the hsa-miR-625-3p gene, a congener thereof, a transcript thereof, or a variant or a derivative thereof. The previously known report shows that change in the expression of the gene or the transcript thereof can serve as a marker for ovarian tumor (Patent Literature 1).

The 266th target gene is the hsa-miR-92a-3p gene, a congener thereof, a transcript thereof, or a variant or a derivative thereof. The previously known report shows that change in the expression of the gene or the transcript thereof can serve as a marker for ovarian tumor (Patent Literature 1).

The 267th target gene is the hsa-miR-422a gene, a congener thereof, a transcript thereof, or a variant or a derivative thereof. The previously known report shows that change in the expression of the gene or the transcript thereof can serve as a marker for ovarian tumor (Non-Patent Literature 3).

The 268th target gene is the hsa-miR-642a-3p gene, a congener thereof, a transcript thereof, or a variant or a derivative thereof. None of the previously known report shows that change in the expression of the gene or the transcript thereof can serve as a marker for ovarian tumor.

The 269th target gene is the hsa-miR-483-5p gene, a congener thereof, a transcript thereof, or a variant or a derivative thereof. The previously known report shows that change in the expression of the gene or the transcript thereof can serve as a marker for ovarian tumor (Patent Literature 1).

The 270th target gene is the hsa-miR-652-5p gene, a congener thereof, a transcript thereof, or a variant or a derivative thereof. The previously known report shows that change in the expression of the gene or the transcript thereof can serve as a marker for ovarian tumor (Patent Literature 4).

The 271st target gene is the hsa-miR-24-3p gene, a congener thereof, a transcript thereof, or a variant or a derivative thereof. The previously known report shows that change in the expression of the gene or the transcript thereof can serve as a marker for ovarian tumor (Patent Literature 1).

The 272nd target gene is the hsa-miR-23b-3p gene, a congener thereof, a transcript thereof, or a variant or a derivative thereof. The previously known report shows that change in the expression of the gene or the transcript thereof can serve as a marker for ovarian tumor (Non-Patent Literature 3).

The 273rd target gene is the hsa-miR-23a-3p gene, a congener thereof, a transcript thereof, or a variant or a derivative thereof. The previously known report shows that change in the expression of the gene or the transcript thereof can serve as a marker for ovarian tumor (Non-Patent Literature 3).

The 274th target gene is the hsa-miR-92b-3p gene, a congener thereof, a transcript thereof, or a variant or a derivative thereof. The previously known report shows that change in the expression of the gene or the transcript thereof can serve as a marker for ovarian tumor (Non-Patent Literature 3).

The 275th target gene is the hsa-miR-22-3p gene, a congener thereof, a transcript thereof, or a variant or a derivative thereof. The previously known report shows that change in the expression of the gene or the transcript thereof can serve as a marker for ovarian tumor (Non-Patent Literature 2).

In one aspect, the present invention relates to a marker containing at least one of the target nucleic acids described above for detecting ovarian tumor or for diagnosing ovarian tumor.

In one aspect, the present invention relates to use of at least one of the target nucleic acids described above for detecting ovarian tumor or for diagnosing ovarian tumor.

### 2. Nucleic Acid for Detection of Ovarian Tumor

In the present invention, the nucleic acids for detecting ovarian tumor, e.g., nucleic acid probes or primers that can be used for diagnosing ovarian tumor enable qualitative and/or quantitative measurement of the presence, expression levels, or existing amounts (abundance) of: human-derived hsa-miR-4675, hsa-miR-4783-3p, hsa-miR-1228-5p, hsa-miR-4532, hsa-miR-6802-5p, hsa-miR-6784-5p, hsa-miR-3940-5p, hsa-miR-1307-3p, hsa-miR-8073, hsa-miR-3184-5p, hsa-miR-1233-5p, hsa-miR-6088, hsa-miR-5195-3p, hsa-miR-320b, hsa-miR-4649-5p, hsa-miR-6800-5p, hsa-miR-1343-3p, hsa-miR-4730, hsa-miR-6885-5p, hsa-miR-5100, hsa-miR-1203, hsa-miR-6756-5p, hsa-miR-373-5p, hsa-miR-1268a, hsa-miR-1260b, hsa-miR-4258, hsa-miR-4697-5p, hsa-miR-1469, hsa-miR-4515, hsa-miR-6861-5p, hsa-miR-6821-5p, hsa-miR-575, hsa-miR-6805-5p, hsa-miR-4758-5p, hsa-miR-3663-3p, hsa-miR-4530, hsa-miR-6798-5p, hsa-miR-6781-5p, hsa-miR-885-3p, hsa-miR-1273g-3p, hsa-miR-4787-3p, hsa-miR-4454, hsa-miR-4706, hsa-miR-1249-3p, hsa-miR-887-3p, hsa-miR-6786-5p, hsa-miR-1238-5p, hsa-miR-6749-5p, hsa-miR-6729-5p, hsa-miR-6825-5p, hsa-miR-663b, hsa-miR-6858-5p, hsa-miR-4690-5p, hsa-miR-6765-5p, hsa-miR-4710, hsa-miR-6775-5p, hsa-miR-371a-5p, hsa-miR-6816-5p, hsa-miR-296-3p, hsa-miR-7977, hsa-miR-8069, hsa-miR-6515-3p, hsa-miR-4687-5p, hsa-miR-1343-5p, hsa-miR-7110-5p, hsa-miR-4525, hsa-miR-3158-5p, hsa-miR-6787-5p, hsa-miR-614, hsa-miR-4689, hsa-miR-1185-2-3p, hsa-miR-1268b, hsa-miR-1228-3p, hsa-miR-1185-1-3p, hsa-miR-940, hsa-miR-939-5p, hsa-miR-6757-5p, hsa-miR-1275, hsa-miR-5001-5p, hsa-miR-6826-5p, hsa-miR-6765-3p, hsa-miR-3679-3p, hsa-miR-4718, hsa-miR-4286, hsa-miR-8059, hsa-miR-4447, hsa-miR-4448, hsa-miR-658, hsa-miR-6766-3p, hsa-miR-197-5p, hsa-miR-6887-5p, hsa-miR-6742-5p, hsa-miR-6729-3p, hsa-miR-5090, hsa-miR-7975, hsa-miR-4505, hsa-miR-6889-5p, hsa-miR-4708-3p, hsa-miR-6131, hsa-miR-1225-3p, hsa-miR-6132, hsa-miR-4734, hsa-miR-3194-3p, hsa-miR-638, hsa-miR-2467-3p, hsa-miR-4728-5p, hsa-miR-5572, hsa-miR-6789-5p, hsa-miR-8063, hsa-miR-4429, hsa-miR-6840-3p, hsa-miR-4476, hsa-miR-675-5p, hsa-miR-711, hsa-miR-6875-5p, hsa-miR-3160-5p, hsa-miR-1908-5p, hsa-miR-6726-5p, hsa-miR-1913, hsa-miR-8071, hsa-miR-3648, hsa-miR-4732-5p, hsa-miR-4787-5p, hsa-miR-3917, hsa-miR-619-5p, hsa-miR-1231, hsa-miR-342-5p, hsa-miR-4433a-5p, hsa-miR-6766-5p, hsa-miR-4707-5p, hsa-miR-7114-5p, hsa-miR-6872-3p, hsa-miR-6780b-5p, hsa-miR-7845-5p, hsa-miR-6798-3p, hsa-miR-665, hsa-miR-6848-5p, hsa-miR-5008-5p, hsa-miR-4294, hsa-miR-6511a-5p, hsa-miR-4435, hsa-miR-4747-3p, hsa-miR-6880-3p, hsa-miR-6869-5p, hsa-miR-7150, hsa-miR-1260a, hsa-miR-6877-5p, hsa-miR-6721-5p, hsa-miR-4656, hsa-miR-1229-5p, hsa-miR-4433a-3p, hsa-miR-4274, hsa-miR-4419b, hsa-miR-4674, hsa-miR-6893-5p, hsa-miR-6763-3p, hsa-miR-6762-5p, hsa-miR-6738-5p, hsa-miR-4513, hsa-miR-6746-5p, hsa-miR-6880-5p, hsa-miR-4736, hsa-miR-718, hsa-miR-6717-5p, hsa-miR-7847-3p, hsa-miR-760, hsa-miR-1199-5p, hsa-miR-6813-5p, hsa-miR-6769a-5p, hsa-miR-1193, hsa-miR-7108-3p, hsa-miR-6741-5p, hsa-miR-4298, hsa-miR-6796-3p, hsa-miR-4750-5p, hsa-miR-6785-5p, hsa-miR-1292-3p, hsa-miR-4749-3p, hsa-miR-6800-3p, hsa-miR-4722-5p, hsa-miR-4746-3p, hsa-miR-4450, hsa-miR-6795-5p, hsa-miR-365a-5p, hsa-miR-498, hsa-miR-6797-5p, hsa-miR-1470, hsa-miR-6851-5p, hsa-miR-1247-3p, hsa-miR-5196-5p, hsa-miR-208a-5p, hsa-miR-6842-5p, hsa-miR-150-3p, hsa-miR-4534, hsa-miR-3135b, hsa-miR-3131, hsa-miR-4792, hsa-miR-6510-5p, hsa-miR-504-3p, hsa-miR-3619-3p, hsa-miR-671-5p, hsa-miR-4667-5p, hsa-miR-4430, hsa-miR-3195, hsa-miR-3679-5p, hsa-miR-6076, hsa-miR-6515-5p, hsa-miR-6820-5p, hsa-miR-4634, hsa-miR-187-5p, hsa-miR-6763-5p, hsa-miR-1908-3p, hsa-miR-1181, hsa-miR-6782-5p, hsa-miR-5010-5p, hsa-miR-6870-5p, hsa-miR-6124, hsa-miR-1249-5p, hsa-miR-6511b-5p, hsa-miR-1254, hsa-miR-4727-3p, hsa-miR-4259, hsa-miR-4771, hsa-miR-3622a-5p, hsa-miR-4480, hsa-miR-4740-5p, hsa-miR-6777-5p, hsa-miR-6794-5p, hsa-miR-4687-3p, hsa-miR-6743-5p, hsa-miR-6771-5p, hsa-miR-3141, hsa-miR-3162-5p, hsa-miR-4271, hsa-miR-1227-5p, hsa-miR-4257, hsa-miR-4270, hsa-miR-4516, hsa-miR-4651, hsa-miR-4725-3p, hsa-miR-6125, hsa-miR-6732-5p, hsa-miR-6791-5p, hsa-miR-6819-5p, hsa-miR-6891-5p, hsa-miR-7108-5p, hsa-miR-7109-5p, hsa-miR-642b-3p and hsa-miR-642a-3p, as target nucleic acids for ovarian tumor, or a combination thereof; and hsa-miR-320a, hsa-miR-663a, hsa-miR-328-5p, hsa-miR-128-2-5p, hsa-miR-125a-3p, hsa-miR-191-5p, hsa-miR-92b-5p, hsa-miR-296-5p, hsa-miR-1246, hsa-miR-92a-2-5p, hsa-miR-128-1-5p, hsa-miR-1290, hsa-miR-211-3p, hsa-miR-744-5p, hsa-miR-135a-3p, hsa-miR-451a, hsa-miR-625-3p, hsa-miR-92a-3p, hsa-miR-422a, hsa-miR-483-5p, hsa-miR-652-5p, hsa-miR-24-3p, hsa-miR-23b-3p, hsa-miR-23a-3p, hsa-miR-92b-3p, hsa-miR-22-3p, or combinations thereof; congeners thereof: transcripts thereof: or variants or derivatives thereof.

The expression levels of the target nucleic acids described above are increased or decreased (hereinafter, referred to as “increased/decreased”) depending on the types of the target nucleic acids in subjects having ovarian tumor as compared with healthy subjects, benign bone and soft tissue tumor patients and benign breast disease patients (or diseased animals), and subjects having a cancer other than ovarian cancer. Hence, the kit or device of the present invention can be effectively used for measuring expression levels of the target nucleic acids in body fluids from subjects (e.g., humans) suspected of having ovarian tumor and body fluids from healthy subjects, benign bone and soft tissue tumor patients and benign breast disease patients (or diseased animals), and patients (or cancer animals) having a cancer other than ovarian cancer, and thereby detecting ovarian tumor through the comparison thereof.

The nucleic acid probe or primer(s) that can be used in the present invention is, for example, a nucleic acid probe capable of specifically binding to a polynucleotide consisting of a nucleotide sequence represented by at least one of SEQ ID NOs: 1 to 247, 251, and 268, or to a complementary strand of the polynucleotide; or a primer(s) for amplifying a polynucleotide consisting of a nucleotide sequence represented by at least one of SEQ ID NOs: 1 to 247, 251, and 268.

The nucleic acid probe or primer(s) that can be used in the present invention may further comprise, for example, a nucleic acid probe capable of specifically binding to a polynucleotide consisting of a nucleotide sequence represented by at least one of SEQ ID NOs: 248 to 250, 252 to 267, and 269 to 275, or to a complementary strand of the polynucleotide; or a primer(s) for amplifying a polynucleotide consisting of a nucleotide sequence represented by at least one of SEQ ID NOs: 248 to 250, 252 to 267, and 269 to 275.

In a preferred embodiment of the present invention, specifically, these nucleic acid probes or primers comprise a combination of one or more polynucleotides selected from: a group of polynucleotides comprising nucleotide sequences represented by any of SEQ ID NOs: 1 to 842 or nucleotide sequences derived from the nucleotide sequences by the replacement of u with t, and a group of complementary polynucleotides thereof; a group of polynucleotides respectively hybridizing under stringent conditions (mentioned later) to DNAs consisting of nucleotide sequences complementary to these nucleotide sequences, and a group of complementary polynucleotides thereof; and a group of polynucleotides comprising 15 or more, preferably 17 or more consecutive nucleotides and being from the nucleotide sequences of these polynucleotide groups. These polynucleotides can be used as nucleic acid probes and primers for detecting the ovarian tumor markers as target nucleic acids.

More specifically, examples of the nucleic acid probes or the primers that can be used in the present invention include one or more polynucleotides selected from the group consisting of the following polynucleotides (a) to (e):
(a) a polynucleotide consisting of a nucleotide sequence represented by any of SEQ ID NOs: 1 to 247, 251, and 268 or a nucleotide sequence derived from the nucleotide sequence by the replacement of u with t, a variant thereof, a derivative thereof, or a fragment thereof comprising 15 or more consecutive nucleotides;
(b) a polynucleotide comprising a nucleotide sequence represented by any of SEQ ID NOs: 1 to 247, 251, and 268;
(c) a polynucleotide consisting of a nucleotide sequence complementary to a nucleotide sequence represented by any of SEQ ID NOs: 1 to 247, 251, and 268 or a nucleotide sequence derived from the nucleotide sequence by the replacement of u with t, a variant thereof, a derivative thereof, or a fragment thereof comprising 15 or more consecutive nucleotides;
(d) a polynucleotide comprising a nucleotide sequence complementary to a nucleotide sequence represented by any of SEQ ID NOs: 1 to 247, 251, and 268 or a nucleotide sequence derived from the nucleotide sequence by the replacement of u with t; and
(e) a polynucleotide hybridizing under stringent conditions to any of the polynucleotides (a) to (d).

In addition to at least one polynucleotide selected from any of the polynucleotides (a) to (e), the nucleic acid probes or the primers that can be used in the present invention may further comprise any of the following polynucleotides (f) to j):
(f) a polynucleotide consisting of a nucleotide sequence represented by any of SEQ ID NOs: 248 to 250, 252 to 267, and 269 to 275 or a nucleotide sequence derived from the nucleotide sequence by the replacement of u with t, a variant thereof, a derivative thereof, or a fragment thereof comprising 15 or more consecutive nucleotides;
(g) a polynucleotide comprising a nucleotide sequence represented by any of SEQ ID NOs: 248 to 250, 252 to 267, and 269 to 275;
(h) a polynucleotide consisting of a nucleotide sequence complementary to a nucleotide sequence represented by any of SEQ ID NOs: 248 to 250, 252 to 267, and 269 to 275 or a nucleotide sequence derived from the nucleotide sequence by the replacement of u with t, a variant thereof, a derivative thereof, or a fragment thereof comprising 15 or more consecutive nucleotides;
(i) a polynucleotide comprising a nucleotide sequence complementary to a nucleotide sequence represented by any of SEQ ID NOs: 248 to 250, 252 to 267, and 269 to 275 or a nucleotide sequence derived from the nucleotide sequence by the replacement of u with t; and
j) a polynucleotide hybridizing under stringent conditions to any of the polynucleotides (f) to (i).

These polynucleotides or fragments thereof used in the present invention may each be DNA or may each be RNA.

The polynucleotides that can be used in the present invention can be prepared by use of a general technique such as a DNA recombination technique, a PCR method, or a method using an automatic DNA/RNA synthesizer.

The DNA recombination technique and the PCR method may employ techniques described in, for example, Ausubel et al., Current Protocols in Molecular Biology, John Willey & Sons, US (1993); and Sambrook et al., Molecular Cloning-A Laboratory Manual, Cold Spring Harbor Laboratory Press, US (1989).

The human-derived hsa-miR-4675, hsa-miR-4783-3p, hsa-miR-1228-5p, hsa-miR-4532, hsa-miR-6802-5p, hsa-miR-6784-5p, hsa-miR-3940-5p, hsa-miR-1307-3p, hsa-miR-8073, hsa-miR-3184-5p, hsa-miR-1233-5p, hsa-miR-6088, hsa-miR-5195-3p, hsa-miR-320b, hsa-miR-4649-5p, hsa-miR-6800-5p, hsa-miR-1343-3p, hsa-miR-4730, hsa-miR-6885-5p, hsa-miR-5100, hsa-miR-1203, hsa-miR-6756-5p, hsa-miR-373-5p, hsa-miR-1268a, hsa-miR-1260b, hsa-miR-4258, hsa-miR-4697-5p, hsa-miR-1469, hsa-miR-4515, hsa-miR-6861-5p, hsa-miR-6821-5p, hsa-miR-575, hsa-miR-6805-5p, hsa-miR-4758-5p, hsa-miR-3663-3p, hsa-miR-4530, hsa-miR-6798-5p, hsa-miR-6781-5p, hsa-miR-885-3p, hsa-miR-1273g-3p, hsa-miR-4787-3p, hsa-miR-4454, hsa-miR-4706, hsa-miR-1249-3p, hsa-miR-887-3p, hsa-miR-6786-5p, hsa-miR-1238-5p, hsa-miR-6749-5p, hsa-miR-6729-5p, hsa-miR-6825-5p, hsa-miR-663b, hsa-miR-6858-5p, hsa-miR-4690-5p, hsa-miR-6765-5p, hsa-miR-4710, hsa-miR-6775-5p, hsa-miR-371a-5p, hsa-miR-6816-5p, hsa-miR-296-3p, hsa-miR-7977, hsa-miR-8069, hsa-miR-6515-3p, hsa-miR-4687-5p, hsa-miR-1343-5p, hsa-miR-7110-5p, hsa-miR-4525, hsa-miR-3158-5p, hsa-miR-6787-5p, hsa-miR-614, hsa-miR-4689, hsa-miR-1185-2-3p, hsa-miR-1268b, hsa-miR-1228-3p, hsa-miR-1185-1-3p, hsa-miR-940, hsa-miR-939-5p, hsa-miR-6757-5p, hsa-miR-1275, hsa-miR-5001-5p, hsa-miR-6826-5p, hsa-miR-6765-3p, hsa-miR-3679-3p, hsa-miR-4718, hsa-miR-4286, hsa-miR-8059, hsa-miR-4447, hsa-miR-4448, hsa-miR-658, hsa-miR-6766-3p, hsa-miR-197-5p, hsa-miR-6887-5p, hsa-miR-6742-5p, hsa-miR-6729-3p, hsa-miR-5090, hsa-miR-7975, hsa-miR-4505, hsa-miR-6889-5p, hsa-miR-4708-3p, hsa-miR-6131, hsa-miR-1225-3p, hsa-miR-6132, hsa-miR-4734, hsa-miR-3194-3p, hsa-miR-638, hsa-miR-2467-3p, hsa-miR-4728-5p, hsa-miR-5572, hsa-miR-6789-5p, hsa-miR-8063, hsa-miR-4429, hsa-miR-6840-3p, hsa-miR-4476, hsa-miR-675-5p, hsa-miR-711, hsa-miR-6875-5p, hsa-miR-3160-5p, hsa-miR-1908-5p, hsa-miR-6726-5p, hsa-miR-1913, hsa-miR-8071, hsa-miR-3648, hsa-miR-4732-5p, hsa-miR-4787-5p, hsa-miR-3917, hsa-miR-619-5p, hsa-miR-1231, hsa-miR-342-5p, hsa-miR-4433a-5p, hsa-miR-6766-5p, hsa-miR-4707-5p, hsa-miR-7114-5p, hsa-miR-6872-3p, hsa-miR-6780b-5p, hsa-miR-7845-5p, hsa-miR-6798-3p, hsa-miR-665, hsa-miR-6848-5p, hsa-miR-5008-5p, hsa-miR-4294, hsa-miR-6511a-5p, hsa-miR-4435, hsa-miR-4747-3p, hsa-miR-6880-3p, hsa-miR-6869-5p, hsa-miR-7150, hsa-miR-1260a, hsa-miR-6877-5p, hsa-miR-6721-5p, hsa-miR-4656, hsa-miR-1229-5p, hsa-miR-4433a-3p, hsa-miR-4274, hsa-miR-4419b, hsa-miR-4674, hsa-miR-6893-5p, hsa-miR-6763-3p, hsa-miR-6762-5p, hsa-miR-6738-5p, hsa-miR-4513, hsa-miR-6746-5p, hsa-miR-6880-5p, hsa-miR-4736, hsa-miR-718, hsa-miR-6717-5p, hsa-miR-7847-3p, hsa-miR-760, hsa-miR-1199-5p, hsa-miR-6813-5p, hsa-miR-6769a-5p, hsa-miR-1193, hsa-miR-7108-3p, hsa-miR-6741-5p, hsa-miR-4298, hsa-miR-6796-3p, hsa-miR-4750-5p, hsa-miR-6785-5p, hsa-miR-1292-3p, hsa-miR-4749-3p, hsa-miR-6800-3p, hsa-miR-4722-5p, hsa-miR-4746-3p, hsa-miR-4450, hsa-miR-6795-5p, hsa-miR-365a-5p, hsa-miR-498, hsa-miR-6797-5p, hsa-miR-1470, hsa-miR-6851-5p, hsa-miR-1247-3p, hsa-miR-5196-5p, hsa-miR-208a-5p, hsa-miR-6842-5p, hsa-miR-150-3p, hsa-miR-4534, hsa-miR-3135b, hsa-miR-3131, hsa-miR-4792, hsa-miR-6510-5p, hsa-miR-504-3p, hsa-miR-3619-3p, hsa-miR-671-5p, hsa-miR-4667-5p, hsa-miR-4430, hsa-miR-3195, hsa-miR-3679-5p, hsa-miR-6076, hsa-miR-6515-5p, hsa-miR-6820-5p, hsa-miR-4634, hsa-miR-187-5p, hsa-miR-6763-5p, hsa-miR-1908-3p, hsa-miR-1181, hsa-miR-6782-5p, hsa-miR-5010-5p, hsa-miR-6870-5p, hsa-miR-6124, hsa-miR-1249-5p, hsa-miR-6511b-5p, hsa-miR-1254, hsa-miR-4727-3p, hsa-miR-4259, hsa-miR-4771, hsa-miR-3622a-5p, hsa-miR-4480, hsa-miR-4740-5p, hsa-miR-6777-5p, hsa-miR-6794-5p, hsa-miR-4687-3p, hsa-miR-6743-5p, hsa-miR-6771-5p, hsa-miR-3141, hsa-miR-3162-5p, hsa-miR-4271, hsa-miR-1227-5p, hsa-miR-4257, hsa-miR-4270, hsa-miR-4516, hsa-miR-4651, hsa-miR-4725-3p, hsa-miR-6125, hsa-miR-6732-5p, hsa-miR-6791-5p, hsa-miR-6819-5p, hsa-miR-6891-5p, hsa-miR-7108-5p, hsa-miR-642b-3p, hsa-miR-642a-3p, hsa-miR-7109-5p, hsa-miR-320a, hsa-miR-663a, hsa-miR-328-5p, hsa-miR-128-2-5p, hsa-miR-125a-3p, hsa-miR-191-5p, hsa-miR-92b-5p, hsa-miR-296-5p, hsa-miR-1246, hsa-miR-92a-2-5p, hsa-miR-128-1-5p, hsa-miR-1290, hsa-miR-211-3p, hsa-miR-744-5p, hsa-miR-135a-3p, hsa-miR-451a, hsa-miR-625-3p, hsa-miR-92a-3p, hsa-miR-422a, hsa-miR-483-5p, hsa-miR-652-5p, hsa-miR-24-3p, hsa-miR-23b-3p, hsa-miR-23a-3p, hsa-miR-92b-3p, and hsa-miR-22-3p represented by SEQ ID NOs: 1 to 275 are known in the art, and their obtainment methods are also known as mentioned above. Therefore, each polynucleotide that can be used as a nucleic acid probe or a primer in the present invention can be prepared by cloning the gene.

Such nucleic acid probes or primers can be chemically synthesized using an automatic DNA synthesizer. In general, the phosphoramidite method is used in this synthesis, and single-stranded DNA up to approximately 100 nucleotides can be automatically synthesized by this method. The automatic DNA synthesizer is commercially available from, for example, Polygen GmbH, ABI, or Applied Biosystems, Inc.

Alternatively, the polynucleotides of the present invention can also be prepared by cDNA cloning methods. The cDNA cloning technique may employ, for example, microRNA Cloning Kit Wako.

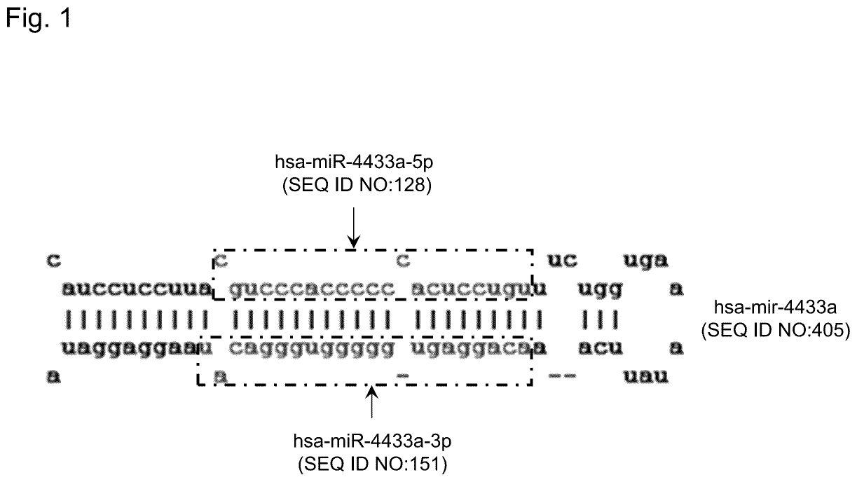

In this context, the sequences of the nucleic acid probes and the primers for detecting the polynucleotide consisting of a nucleotide sequence represented by any of SEQ ID NOs: 1 to 275 do not exist as miRNAs or precursors thereof in the living body or in vivo. For example, the nucleotide sequences represented by SEQ ID NO: 128 and SEQ ID NO: 151 are produced from the precursor represented by SEQ ID NO: 405. This precursor has a hairpin-like structure as shown in FIG. 1, and the nucleotide sequences represented by SEQ ID NO: 128 and SEQ ID NO: 151 have mismatch sequences with each other. As such, a nucleotide sequence completely complementary to the nucleotide sequence represented by SEQ ID NO: 121 or SEQ ID NO: 151 does not naturally occur in vivo. Therefore, the nucleic acid probes and the primers for detecting the nucleotide sequence represented by any of SEQ ID NOs: 1 to 275 can have artificial nucleotide sequences that do not exist in the living body or in vivo.

### 3. Kit or Device for Detection of Ovarian Tumor

The present invention also provides a kit or a device for detection of ovarian tumor, comprising one or more polynucleotides (which may include a variant, a fragment, or a derivative thereof) that can be used as nucleic acid probes or primers in the present invention for measuring target nucleic acids as ovarian tumor markers.

The target nucleic acids as ovarian tumor markers according to the present invention are preferably selected from the following group A:

### Group A:

hsa-miR-4675, hsa-miR-4783-3p, hsa-miR-1228-5p, hsa-miR-4532, hsa-miR-6802-5p, hsa-miR-6784-5p, hsa-miR-3940-5p, hsa-miR-1307-3p, hsa-miR-8073, hsa-miR-3184-5p, hsa-miR-1233-5p, hsa-miR-6088, hsa-miR-5195-3p, hsa-miR-320b, hsa-miR-4649-5p, hsa-miR-6800-5p, hsa-miR-1343-3p, hsa-miR-4730, hsa-miR-6885-5p, hsa-miR-5100, hsa-miR-1203, hsa-miR-6756-5p, hsa-miR-373-5p, hsa-miR-1268a, hsa-miR-1260b, hsa-miR-4258, hsa-miR-4697-5p, hsa-miR-1469, hsa-miR-4515, hsa-miR-6861-5p, hsa-miR-6821-5p, hsa-miR-575, hsa-miR-6805-5p, hsa-miR-4758-5p, hsa-miR-3663-3p, hsa-miR-4530, hsa-miR-6798-5p, hsa-miR-6781-5p, hsa-miR-885-3p, hsa-miR-1273g-3p, hsa-miR-4787-3p, hsa-miR-4454, hsa-miR-4706, hsa-miR-1249-3p, hsa-miR-887-3p, hsa-miR-6786-5p, hsa-miR-1238-5p, hsa-miR-6749-5p, hsa-miR-6729-5p, hsa-miR-6825-5p, hsa-miR-663b, hsa-miR-6858-5p, hsa-miR-4690-5p, hsa-miR-6765-5p, hsa-miR-4710, hsa-miR-6775-5p, hsa-miR-371a-5p, hsa-miR-6816-5p, hsa-miR-296-3p, hsa-miR-7977, hsa-miR-8069, hsa-miR-6515-3p, hsa-miR-4687-5p, hsa-miR-1343-5p, hsa-miR-7110-5p, hsa-miR-4525, hsa-miR-3158-5p, hsa-miR-6787-5p, hsa-miR-614, hsa-miR-4689, hsa-miR-1185-2-3p, hsa-miR-1268b, hsa-miR-1228-3p, hsa-miR-1185-1-3p, hsa-miR-940, hsa-miR-939-5p, hsa-miR-6757-5p, hsa-miR-1275, hsa-miR-5001-5p, hsa-miR-6826-5p, hsa-miR-6765-3p, hsa-miR-3679-3p, hsa-miR-4718, hsa-miR-4286, hsa-miR-8059, hsa-miR-4447, hsa-miR-4448, hsa-miR-658, hsa-miR-6766-3p, hsa-miR-197-5p, hsa-miR-6887-5p, hsa-miR-6742-5p, hsa-miR-6729-3p, hsa-miR-5090, hsa-miR-7975, hsa-miR-4505, hsa-miR-6889-5p, hsa-miR-4708-3p, hsa-miR-6131, hsa-miR-1225-3p, hsa-miR-6132, hsa-miR-4734, hsa-miR-3194-3p, hsa-miR-638, hsa-miR-2467-3p, hsa-miR-4728-5p, hsa-miR-5572, hsa-miR-6789-5p, hsa-miR-8063, hsa-miR-4429, hsa-miR-6840-3p, hsa-miR-4476, hsa-miR-675-5p, hsa-miR-711, hsa-miR-6875-5p, hsa-miR-3160-5p, hsa-miR-1908-5p, hsa-miR-6726-5p, hsa-miR-1913, hsa-miR-8071, hsa-miR-3648, hsa-miR-4732-5p, hsa-miR-4787-5p, hsa-miR-3917, hsa-miR-619-5p, hsa-miR-1231, hsa-miR-342-5p, hsa-miR-4433a-5p, hsa-miR-6766-5p, hsa-miR-4707-5p, hsa-miR-7114-5p, hsa-miR-6872-3p, hsa-miR-6780b-5p, hsa-miR-7845-5p, hsa-miR-6798-3p, hsa-miR-665, hsa-miR-6848-5p, hsa-miR-5008-5p, hsa-miR-4294, hsa-miR-6511a-5p, hsa-miR-4435, hsa-miR-4747-3p, hsa-miR-6880-3p, hsa-miR-6869-5p, hsa-miR-7150, hsa-miR-1260a, hsa-miR-6877-5p, hsa-miR-6721-5p, hsa-miR-4656, hsa-miR-1229-5p, hsa-miR-4433a-3p, hsa-miR-4274, hsa-miR-4419b, hsa-miR-4674, hsa-miR-6893-5p, hsa-miR-6763-3p, hsa-miR-6762-5p, hsa-miR-6738-5p, hsa-miR-4513, hsa-miR-6746-5p, hsa-miR-6880-5p, hsa-miR-4736, hsa-miR-718, hsa-miR-6717-5p, hsa-miR-7847-3p, hsa-miR-760, hsa-miR-1199-5p, hsa-miR-6813-5p, hsa-miR-6769a-5p, hsa-miR-1193, hsa-miR-7108-3p, hsa-miR-6741-5p, hsa-miR-4298, hsa-miR-6796-3p, hsa-miR-4750-5p, hsa-miR-6785-5p, hsa-miR-1292-3p, hsa-miR-4749-3p, hsa-miR-6800-3p, hsa-miR-4722-5p, hsa-miR-4746-3p, hsa-miR-4450, hsa-miR-6795-5p, hsa-miR-365a-5p, hsa-miR-498, hsa-miR-6797-5p, hsa-miR-1470, hsa-miR-6851-5p, hsa-miR-1247-3p, hsa-miR-5196-5p, hsa-miR-208a-5p, hsa-miR-6842-5p, hsa-miR-150-3p, hsa-miR-4534, hsa-miR-3135b, hsa-miR-3131, hsa-miR-4792, hsa-miR-6510-5p, hsa-miR-504-3p, hsa-miR-3619-3p, hsa-miR-671-5p, hsa-miR-4667-5p, hsa-miR-4430, hsa-miR-3195, hsa-miR-3679-5p, hsa-miR-6076, hsa-miR-6515-5p, hsa-miR-6820-5p, hsa-miR-4634, hsa-miR-187-5p, hsa-miR-6763-5p, hsa-miR-1908-3p, hsa-miR-1181, hsa-miR-6782-5p, hsa-miR-5010-5p, hsa-miR-6870-5p, hsa-miR-6124, hsa-miR-1249-5p, hsa-miR-6511b-5p, hsa-miR-1254, hsa-miR-4727-3p, hsa-miR-4259, hsa-miR-4771, hsa-miR-3622a-5p, hsa-miR-4480, hsa-miR-4740-5p, hsa-miR-6777-5p, hsa-miR-6794-5p, hsa-miR-4687-3p, hsa-miR-6743-5p, hsa-miR-6771-5p, hsa-miR-3141, hsa-miR-3162-5p, hsa-miR-4271, hsa-miR-1227-5p, hsa-miR-4257, hsa-miR-4270, hsa-miR-4516, hsa-miR-4651, hsa-miR-4725-3p, hsa-miR-6125, hsa-miR-6732-5p, hsa-miR-6791-5p, hsa-miR-6819-5p, hsa-miR-6891-5p, hsa-miR-7108-5p, hsa-miR-7109-5p, hsa-miR-642b-3p, and hsa-miR-642a-3p.

Additional target nucleic acids that may be optionally used in the measurement are preferably selected from the following group B:

### Group B:

hsa-miR-320a, hsa-miR-663a, hsa-miR-328-5p, hsa-miR-128-2-5p, hsa-miR-125a-3p, hsa-miR-191-5p, hsa-miR-92b-5p, hsa-miR-296-5p, hsa-miR-1246, hsa-miR-92a-2-5p, hsa-miR-128-1-5p, hsa-miR-1290, hsa-miR-211-3p, hsa-miR-744-5p, hsa-miR-135a-3p, hsa-miR-451a, hsa-miR-625-3p, hsa-miR-92a-3p, hsa-miR-422a, hsa-miR-483-5p, hsa-miR-652-5p, hsa-miR-24-3p, hsa-miR-23b-3p, hsa-miR-23a-3p, hsa-miR-92b-3p, hsa-miR-22-3p.

The kit or the device of the present invention comprises one or more nucleic acids capable of specifically binding to any of the target nucleic acids as the ovarian tumor markers described above or nucleic acids for detecting the target nucleic acids, preferably one or more polynucleotides selected from the polynucleotides described in the preceding Section 2, or variants thereof.

Specifically, the kit or the device of the present invention can comprise at least one polynucleotide comprising (or consisting of), for example, a nucleotide sequence represented by any of SEQ ID NOs: 1 to 247, 251, and 268 or a nucleotide sequence derived from the nucleotide sequence by the replacement of u with t, polynucleotide(s) comprising (or consisting of) a complementary sequence thereof, a polynucleotide(s) hybridizing under stringent conditions to any of these polynucleotides, or a variant(s) or a fragment(s) comprising 15 or more consecutive nucleotides of any of these polynucleotide sequences.

The kit or the device of the present invention can further comprise one or more polynucleotides comprising (or consisting of), for example, a nucleotide sequence represented by any of SEQ ID NOs: 248 to 250, 252 to 267, and 269 to 275 or a nucleotide sequence derived from the nucleotide sequence by the replacement of u with t, a polynucleotide(s) comprising (or consisting of) a complementary sequence thereof, a polynucleotide(s) hybridizing under stringent conditions to any of these polynucleotides, a variant(s), or a fragment(s) comprising 15 or more consecutive nucleotides of any of these polynucleotide sequences.

The fragment or fragments that can be comprised in the kit or the device of the present invention is/are, for example, one or more polynucleotides, preferably two or more polynucleotides, selected from the group consisting of the following polynucleotides (1) and (2):
(1) a polynucleotide comprising 15 or more consecutive nucleotides that are from a nucleotide sequence derived from a nucleotide sequence represented by any of SEQ ID NOs: 1 to 247, 251, and 268 by the replacement of u with t, or a complementary sequence thereof; and
(2) a polynucleotide comprising 15 or more consecutive nucleotides that are from a nucleotide sequence derived from a nucleotide sequence represented by any of SEQ ID NOs: 248 to 250, 252 to 267, and 269 to 275 by the replacement of u with t, or a complementary sequence thereof.

In a preferred embodiment, the polynucleotide is a polynucleotide consisting of a nucleotide sequence represented by any of SEQ ID NOs: 1 to 247, 251, and 268 or a nucleotide sequence derived from the nucleotide sequence by the replacement of u with t, a polynucleotide consisting of a complementary sequence thereof, a polynucleotide hybridizing under stringent conditions to any of these polynucleotides, or a variant thereof comprising 15 or more, preferably 17 or more, more preferably 19 or more consecutive nucleotides.

In a preferred embodiment, the polynucleotide is a polynucleotide consisting of a nucleotide sequence represented by any of SEQ ID NOs: 248 to 250, 252 to 267, and 269 to 275 or a nucleotide sequence derived from the nucleotide sequence by the replacement of u with t, a polynucleotide consisting of a complementary sequence thereof, a polynucleotide hybridizing under stringent conditions to any of these polynucleotides, or a variant thereof comprising 15 or more, preferably 17 or more, more preferably 19 or more consecutive nucleotides.

In a preferred embodiment, the fragment can be a polynucleotide comprising 15 or more, preferably 17 or more, more preferably 19 or more consecutive nucleotides.

In the present invention, the size of the polynucleotide fragment is the number of nucleotides in the range from, for example, 15 consecutive nucleotides to less than the total number of nucleotides of the sequence, from 17 consecutive nucleotides to less than the total number of nucleotides of the sequence, or from 19 consecutive nucleotides to less than the total number of nucleotides of the sequence, in the nucleotide sequence of each polynucleotide.

Examples of the combination of the above-mentioned polynucleotides as target nucleic acids in the kit or the device of the present invention can include a single (one) polynucleotide or combinations of 2, 3, 4, 5, 6, 7, 8, 9, 10 or more of the above-mentioned polynucleotides consisting of the nucleotide sequences represented by SEQ ID NOs: 1 to 275 as shown in Table 1 above. However, these are given merely for illustrative purposes, and all of various other possible combinations are included in the present invention.

Examples of the combinations of target nucleic acids in the kit or the device for discriminating ovarian tumor patients from healthy subjects according to the present invention can include, for example, combinations of two or more of the above-mentioned polynucleotides consisting of the nucleotide sequences represented by SEQ ID NOs shown in Table 1. More preferably, the examples can include combination of two or more of the above-mentioned polynucleotides consisting of the nucleotide sequences represented by SEQ ID NOs as shown in Table 2. For example, any two or more of the above-mentioned polynucleotides consisting of nucleotide sequences represented by SEQ ID NOs: 1 to 203 and 248 to 268 can be combined. Among them, at least one polynucleotide of the newly found polynucleotides consisting of the nucleotide sequences represented by SEQ ID NOs: 1 to 203, 251, and 268 is preferably selected.

Additionally, examples of the combinations of target nucleic acids in the kit or the device for discriminating ovarian tumor patients from benign bone and soft tissue tumor patients and benign breast disease patients according to the present invention can include combinations of two or more of the above-mentioned polynucleotides consisting of the nucleotide sequences represented by SEQ ID NOs as shown in Table 1. More preferably, the examples can include combinations of two or more of the above-mentioned polynucleotides consisting of the nucleotide sequences represented by SEQ ID NOs as shown in Table 6. For example, any two of the polynucleotides selected from the group consisting of the nucleotide sequences represented by SEQ ID NOs: 4, 9, 14, 17, 18, 20, 29, 40, 47, 62, 66, 69, 78, 82, 83, 86, 87, 89, 116, 125, 127, 132, 135, 136, 147, 163, 164, 167, 174, 177, 184, 193, 204 to 215, 248, 256, 260, 264, 265, 268, 269, and 270 can be combined. Among them, at least one polynucleotide of the newly found polynucleotides consisting of the nucleotide sequences represented by SEQ ID NOs: 4, 9, 14, 17, 18, 20, 29, 40, 47, 62, 66, 69, 78, 82, 83, 86, 87, 89, 116, 125, 127, 132, 135, 136, 147, 163, 164, 167, 174, 177, 184, 193, 204 to 215, and 268 is preferably selected.

Moreover, examples of the combinations of target nucleic acids in the kit or the device for discriminating ovarian tumor patients from patients having a cancer other than ovarian cancer according to the present invention can include combinations of two or more of the above-mentioned polynucleotides consisting of the nucleotide sequences represented by SEQ ID NOs as shown in Table 1. More preferably, two or more of the above-mentioned polynucleotides consisting of the nucleotide sequences represented by SEQ ID NOs as shown in Table 10 can be combined. For example, any two of the polynucleotides selected from the group consisting of the nucleotide sequences represented by SEQ ID NOs: 4, 11, 17, 68, 69, 76, 78, 82, 85, 89, 99, 103, 105, 109, 127, 128, 135, 145, 160, 164, 169, 196, 198, 207, 211, 216 to 227, 264, 266, 267, 268, 271, 272, 273, 274, and 275 can be combined. Among them, at least one polynucleotide of the newly found polynucleotides selected from the group consisting of the nucleotide sequences represented by SEQ ID NOs: 4, 11, 17, 68, 69, 76, 78, 82, 85, 89, 99, 103, 105, 109, 127, 128, 135, 145, 160, 164, 169, 196, 198, 207, 211, 216 to 227, and 268 is preferably selected.

Besides, the combination of polynucleotides as target nucleic acids with specificity capable of discriminating an ovarian tumor patient from subjects such as healthy subjects, benign bone and soft tissue tumor patients and benign breast disease patients, and patients having a cancer other than ovarian cancer is preferably, for example, a combination of multiple polynucleotides comprising: at least one polynucleotide selected from the group consisting of polynucleotides consisting of the nucleotide sequences represented by SEQ ID NOs: 2, 3, 4, 9, 11, 13, 15, 20, 33, 34, 38, 40, 44, 47, 56, 62, 68, 77, 78, 80, 82, 86, 89, 90, 91, 102, 104, 109, 117, 118, 135, 136, 145, 150, 157, 160, 161, 164, 169, 172, 196, 199, 211, 216, 217, 218, 220, 228, 229, 230, 231, 232, 233, 234, 235, 236, 237, 238, 239, 240, 241, 242, 243, 244, 245, 246, 247, 250, 252, 256, 260, 267, 268 (hereinafter, this group is referred to as “specific polynucleotide group 1”); and any of the polynucleotides consisting of the nucleotide sequences represented by the other SEQ ID NOs.

Furthermore, all of the combinations of polynucleotides as target nucleic acids with cancer type specificity capable of discriminating an ovarian tumor patient not only from a healthy subject but also from a cancer patient other than ovarian cancer are more preferably combinations of multiple polynucleotides (i.e., two or more) selected from the specific polynucleotide group 1.

Moreover, the combination of polynucleotides as target nucleic acids with cancer type specificity capable of discriminating a ovarian tumor patient not only from a healthy subject but also from a cancer patient other than ovarian cancer is more preferably a combination comprising at least one polynucleotide selected from, for example, the group consisting of polynucleotides of SEQ ID NOs: 228, 9, 196, 229, 145, and 164 (hereinafter, this group is referred to as “specific polynucleotide group 2”), among the combinations of multiple polynucleotides selected from the specific polynucleotide group 1.
1. The number of the polynucleotides as target nucleic acids, with cancer type specificity may be s single polynucleotide or any one of 2, 3, 4, 5, 6, 7, 8, 9, 10 or more in the combination. It is preferably 2 or more in the combination.

Non-limiting examples of the combination of the polynucleotide consisting of the nucleotide sequence represented by SEQ ID NO: 228 or a complementary sequence thereof, with polynucleotides selected from the specific polynucleotide group 1 or polynucleotides consisting of complementary sequences thereof are listed below as combinations of target nucleic acids:
(1) a combination of SEQ ID NOs: 256, and 228;
(2) a combination of SEQ ID NOs: 47, and 228;
(3) a combination of SEQ ID NOs: 228, 9, and 229;
(4) a combination of SEQ ID NOs: 228, 9, and 78;
(5) a combination of SEQ ID NOs: 228, 9, and 82;
(6) a combination of SEQ ID NOs: 228, 9, and 268;
(7) a combination of SEQ ID NOs: 229, 260, and 228;
(8) a combination of SEQ ID NOs: 228, 9, and 247;
(9) a combination of SEQ ID NOs: 228, 9, and 62;
(10) a combination of SEQ ID NOs: 228, 9, and 217;
(11) a combination of SEQ ID NOs: 256, 228, and 9;
(12) a combination of SEQ ID NOs: 256, 228, and 260;
(13) a combination of SEQ ID NOs: 228, 9, and 234;
(14) a combination of SEQ ID NOs: 229, 20, and 228;
(15) a combination of SEQ ID NOs: 47, 228, and 82;
(16) a combination of SEQ ID NOs: 145, 260, and 228;
(17) a combination of SEQ ID NOs: 228, 9, and 145;
(18) a combination of SEQ ID NOs: 228, 9, 268, and 160;
(19) a combination of SEQ ID NOs: 228, 9, 82, and 268;
(20) a combination of SEQ ID NOs: 228, 9, 268, and 196;
(21) a combination of SEQ ID NOs: 145, 260, 228, and 250;
(22) a combination of SEQ ID NOs: 145, 260, 228, and 62;
(23) a combination of SEQ ID NOs: 228, 9, 268, and 230;
(24) a combination of SEQ ID NOs: 229, 260, 228, and 2;
(25) a combination of SEQ ID NOs: 228, 9, 268, and 62;
(26) a combination of SEQ ID NOs: 229, 260, 228, and 172;
(27) a combination of SEQ ID NOs: 229, 260, 228, and 4;
(28) a combination of SEQ ID NOs: 229, 260, 228, and 34;
(29) a combination of SEQ ID NOs: 229, 260, 228, and 56;
(30) a combination of SEQ ID NOs: 229, 20, 228, and 4;
(31) a combination of SEQ ID NOs: 229, 20, 228, and 102;
(32) a combination of SEQ ID NOs: 145, 260, 228, and 89;
(33) a combination of SEQ ID NOs: 228, 9, 229, and 82;
(34) a combination of SEQ ID NOs: 228, 9, 78, and 62;
(35) a combination of SEQ ID NOs: 229, 260, 228, and 157;
(36) a combination of SEQ ID NOs: 229, 260, 228, and 231;
(37) a combination of SEQ ID NOs: 256, 228, 260, and 232;
(38) a combination of SEQ ID NOs: 228, 9, 229, and 117;
(39) a combination of SEQ ID NOs: 228, 9, 229, and 62;
(40) a combination of SEQ ID NOs: 228, 9, 78, and 172;
(41) a combination of SEQ ID NOs: 228, 9, 78, and 3;
(42) a combination of SEQ ID NOs: 228, 9, 82, 268, and 196;
(43) a combination of SEQ ID NOs: 229, 20, 228, 4, and 172;
(44) a combination of SEQ ID NOs: 228, 9, 82, 268, and 233;
(45) a combination of SEQ ID NOs: 228, 9, 268, 196, and 89;
(46) a combination of SEQ ID NOs: 228, 9, 268, 160, and 82;
(47) a combination of SEQ ID NOs: 228, 9, 268, 196, and 256;
(48) a combination of SEQ ID NOs: 228, 9, 78, 62, and 238;
(49) a combination of SEQ ID NOs: 228, 9, 268, 196, and 172;
(50) a combination of SEQ ID NOs: 256, 228, 260, 232, and 196;
(51) a combination of SEQ ID NOs: 228, 9, 268, 160, and 230;
(52) a combination of SEQ ID NOs: 256, 196, 260, 268, and 228;
(53) a combination of SEQ ID NOs: 228, 9, 268, 196, and 62;
(54) a combination of SEQ ID NOs: 228, 9, 268, 196, and 244;
(55) a combination of SEQ ID NOs: 228, 9, 268, 196, and 135;
(56) a combination of SEQ ID NOs: 229, 260, 228, 2, and 217;
(57) a combination of SEQ ID NOs: 228, 9, 268, 62, and 233;
(58) a combination of SEQ ID NOs: 229, 260, 228, 172, and 230;
(59) a combination of SEQ ID NOs: 229, 260, 228, 56, and 33;
(60) a combination of SEQ ID NOs: 229, 260, 228, 56, and 231;
(61) a combination of SEQ ID NOs: 229, 260, 228, 231, and 91;
(62) a combination of SEQ ID NOs: 228, 9, 268, 160, and 245;
(63) a combination of SEQ ID NOs: 228, 9, 82, 268, and 237; and
(64) a combination of SEQ ID NOs: 228, 9, 82, 268, and 236.

Non-limiting examples of the combination of, for example, the polynucleotide consisting of the nucleotide sequence represented by SEQ ID NO: 9 or a polynucleotide consisting of a complementary sequence thereof, with polynucleotides selected from the specific polynucleotide group 1 or polynucleotides consisting of complementary sequences thereof are further listed below as combinations of target nucleic acids:
(1) a combination of SEQ ID NOs: 228, 9, and 229;
(2) a combination of SEQ ID NOs: 228, 9, and 78;
(3) a combination of SEQ ID NOs: 228, 9, and 82;
(4) a combination of SEQ ID NOs: 228, 9, and 268;
(5) a combination of SEQ ID NOs: 228, 9, and 247;
(6) a combination of SEQ ID NOs: 228, 9, and 62;
(7) a combination of SEQ ID NOs: 228, 9, and 217;
(8) a combination of SEQ ID NOs: 256, 228, and 9;
(9) a combination of SEQ ID NOs: 228, 9, and 234;
(10) a combination of SEQ ID NOs: 228, 9, and 145;
(11) a combination of SEQ ID NOs: 256, 196, and 9;
(12) a combination of SEQ ID NOs: 228, 9, 268, and 160;
(13) a combination of SEQ ID NOs: 256, 196, 9, and 68;
(14) a combination of SEQ ID NOs: 228, 9, 82, and 268;
(15) a combination of SEQ ID NOs: 228, 9, 268, and 196;
(16) a combination of SEQ ID NOs: 228, 9, 268, and 230;
(17) a combination of SEQ ID NOs: 228, 9, 268, and 62;
(18) a combination of SEQ ID NOs: 228, 9, 229, and 82;
(19) a combination of SEQ ID NOs: 228, 9, 78, and 62;
(20) a combination of SEQ ID NOs: 228, 9, 229, and 117;
(21) a combination of SEQ ID NOs: 228, 9, 229, and 62;
(22) a combination of SEQ ID NOs: 228, 9, 78, and 172;
(23) a combination of SEQ ID NOs: 228, 9, 78, and 3;
(24) a combination of SEQ ID NOs: 228, 9, 82, 268, and 196;
(25) a combination of SEQ ID NOs: 228, 9, 82, 268, and 233;
(26) a combination of SEQ ID NOs: 228, 9, 268, 196, and 89;
(27) a combination of SEQ ID NOs: 228, 9, 268, 160, and 82;
(28) a combination of SEQ ID NOs: 256, 196, 9, 68, and 86;
(29) a combination of SEQ ID NOs: 228, 9, 268, 196, and 256;
(30) a combination of SEQ ID NOs: 228, 9, 78, 62, and 238;
(31) a combination of SEQ ID NOs: 228, 9, 268, 196, and 172;
(32) a combination of SEQ ID NOs: 228, 9, 268, 160, and 230;
(33) a combination of SEQ ID NOs: 228, 9, 268, 196, and 62;
(34) a combination of SEQ ID NOs: 228, 9, 268, 196, and 244;
(35) a combination of SEQ ID NOs: 228, 9, 268, 196, and 135;
(36) a combination of SEQ ID NOs: 228, 9, 268, 62, and 233;
(37) a combination of SEQ ID NOs: 228, 9, 268, 160, and 245;
(38) a combination of SEQ ID NOs: 256, 196, 9, 68, and 268;
(39) a combination of SEQ ID NOs: 228, 9, 82, 268, and 237; and
(40) a combination of SEQ ID NOs: 228, 9, 82, 268, and 236.

Non-limiting examples of the combination of, for example, the polynucleotide consisting of the nucleotide sequence represented by SEQ ID NO: 196 or a complementary sequence thereof, with polynucleotides selected from the specific polynucleotide group 1 or polynucleotides consisting of complementary sequences thereof are further listed below as combinations of target nucleic acids:
(1) a combination of SEQ ID NOs: 256, and 196;
(2) a combination of SEQ ID NOs: 256, 196, and 260;
(3) a combination of SEQ ID NOs: 256, 196, and 172;
(4) a combination of SEQ ID NOs: 256, 196, and 47;
(5) a combination of SEQ ID NOs: 256, 196, and 11;
(6) a combination of SEQ ID NOs: 256, 196, and 136;
(7) a combination of SEQ ID NOs: 256, 196, and 80;
(8) a combination of SEQ ID NOs: 256, 196, and 9;
(9) a combination of SEQ ID NOs: 256, 196, 260, and 268;
(10) a combination of SEQ ID NOs: 256, 196, 9, and 68;
(11) a combination of SEQ ID NOs: 256, 196, 260, and 216;
(12) a combination of SEQ ID NOs: 256, 196, 260, and 11;
(13) a combination of SEQ ID NOs: 228, 9, 268, and 196;
(14) a combination of SEQ ID NOs: 256, 196, 260, and 217;
(15) a combination of SEQ ID NOs: 228, 9, 82, 268, and 196;
(16) a combination of SEQ ID NOs: 228, 9, 268, 196, and 89;
(17) a combination of SEQ ID NOs: 256, 196, 9, 68, and 86;
(18) a combination of SEQ ID NOs: 228, 9, 268, 196, and 256;
(19) a combination of SEQ ID NOs: 256, 196, 260, 268, and 252;
(20) a combination of SEQ ID NOs: 228, 9, 268, 196, and 172;
(21) a combination of SEQ ID NOs: 256, 228, 260, 232, and 196;
(22) a combination of SEQ ID NOs: 256, 196, 260, 268, and 228;
(23) a combination of SEQ ID NOs: 256, 196, 260, 268, and 218;
(24) a combination of SEQ ID NOs: 256, 196, 260, 268, and 267;
(25) a combination of SEQ ID NOs: 228, 9, 268, 196, and 62;
(26) a combination of SEQ ID NOs: 228, 9, 268, 196, and 244;
(27) a combination of SEQ ID NOs: 228, 9, 268, 196, and 135;
(28) a combination of SEQ ID NOs: 256, 196, 260, 217, and 199;
(29) a combination of SEQ ID NOs: 256, 196, 260, 268, and 161; and
(30) a combination of SEQ ID NOs: 256, 196, 9, 68, and 268.

Non-limiting examples of the combination of, for example, the polynucleotide consisting of the nucleotide sequence represented by SEQ ID NO: 229 or a complementary sequence thereof, with polynucleotides selected from the specific polynucleotide group 1 or polynucleotides consisting of complementary sequences thereof are further listed below as combinations of target nucleic acids:
(1) a combination of SEQ ID NOs: 228, 9, and 229;
(2) a combination of SEQ ID NOs: 229, 260, and 228;
(3) a combination of SEQ ID NOs: 229, 260, and 230;
(4) a combination of SEQ ID NOs: 229, 260, and 169;
(5) a combination of SEQ ID NOs: 229, 260, and 160;
(6) a combination of SEQ ID NOs: 229, 20, and 228;
(7) a combination of SEQ ID NOs: 229, 260, 228, and 2;
(8) a combination of SEQ ID NOs: 229, 260, 169, and 2;
(9) a combination of SEQ ID NOs: 229, 260, 228, and 172;
(10) a combination of SEQ ID NOs: 229, 260, 228, and 4;
(11) a combination of SEQ ID NOs: 229, 260, 228, and 34;
(12) a combination of SEQ ID NOs: 229, 260, 228, and 56;
(13) a combination of SEQ ID NOs: 229, 20, 228, and 4;
(14) a combination of SEQ ID NOs: 229, 20, 228, and 102;
(15) a combination of SEQ ID NOs: 228, 9, 229, and 82;
(16) a combination of SEQ ID NOs: 229, 260, 228, and 157;
(17) a combination of SEQ ID NOs: 229, 260, 228, and 231;
(18) a combination of SEQ ID NOs: 228, 9, 229, and 117;
(19) a combination of SEQ ID NOs: 228, 9, 229, and 62;
(20) a combination of SEQ ID NOs: 229, 20, 228, 4, and 172;
(21) a combination of SEQ ID NOs: 229, 260, 228, 2, and 217;
(22) a combination of SEQ ID NOs: 229, 260, 228, 172, and 230;
(23) a combination of SEQ ID NOs: 229, 260, 228, 56, and 33;
(24) a combination of SEQ ID NOs: 229, 260, 228, 56, and 231; and
(25) a combination of SEQ ID NOs: 229, 260, 228, 231, and 91.

Non-limiting examples of the combination of, for example, the polynucleotide consisting of the nucleotide sequence represented by SEQ ID NO: 145 or a complementary sequence thereof, with polynucleotides selected from the specific polynucleotide group 1 or polynucleotides consisting of complementary sequences thereof are further listed below as combinations of target nucleic acids:
(1) a combination of SEQ ID NOs: 145, 260, and 164;
(2) a combination of SEQ ID NOs: 145, 260, and 230;
(3) a combination of SEQ ID NOs: 145, 260, and 211;
(4) a combination of SEQ ID NOs: 145, 260, and 228;
(5) a combination of SEQ ID NOs: 228, 9, and 145;
(6) a combination of SEQ ID NOs: 145, 260, 228, and 250;
(7) a combination of SEQ ID NOs: 145, 260, 228, and 62; and
(8) a combination of SEQ ID NOs: 145, 260, 228, and 89.

Non-limiting examples of the combination of, for example, the polynucleotide consisting of the nucleotide sequence represented by SEQ ID NO: 164 or a complementary sequence thereof, with polynucleotides selected from the specific polynucleotide group 1 or polynucleotides consisting of complementary sequences thereof are further listed below as combinations of target nucleic acids:
(1) a combination of SEQ ID NOs: 11, 164, and 260;
(2) a combination of SEQ ID NOs: 11, 164, and 20; and
(3) a combination of SEQ ID NOs: 145, 260, and 164.

The kit or device of the present invention can also comprise polynucleotide(s) which can detect ovarian tumor and are known in the art or will be found in the future in addition to the polynucleotide(s) (that can comprise variant(s), fragments, or derivative(s)) according to the present invention as described above.

The kit or device of the present invention can also comprise an antibody for measuring a marker or markers for ovarian tumor examination known in the art, such as CA-125, in addition to the polynucleotide(s) according to the present invention as described above, and a variant or variants thereof or a fragment or fragments thereof.

These polynucleotides and variants thereof or fragments thereof contained in the kit of the present invention may be packaged in different containers either individually or in any combination.

The kit of the present invention may comprise a kit for extracting nucleic acids (e.g., total RNA) from body fluids, cells, or tissues, a fluorescent material for labeling, an enzyme and a medium for nucleic acid amplification, an instruction manual, etc.

The device of the present invention is a device for measurement of cancer markers in which nucleic acids such as the polynucleotides according to the present invention described above, variants thereof, derivatives thereof, or fragments thereof are bonded or attached to, for example, a solid phase. Examples of the material for the solid phase include plastics, paper, glass, and silicon. The material for the solid phase is preferably a plastic from the viewpoint of easy processability. The solid phase has any shape and is, for example, square, round, reed-shaped, or film-shaped. The device of the present invention includes, for example, a device for measurement by a hybridization technique. Specific examples thereof include blotting devices and nucleic acid arrays (e.g., microarrays, DNA chips, and RNA chips).

The nucleic acid array technique is a technique which involves bonding or attaching the nucleic acids one by one by use of a method [e.g., a method of spotting the nucleic acids using a high-density dispenser called spotter or arrayer onto the surface of the solid phase surface-treated, if necessary, by coating with L-lysine or the introduction of a functional group such as an amino group or a carboxyl group, a method of spraying the nucleic acids onto the solid phase using an inkjet which injects very small liquid droplets by a piezoelectric element or the like from a nozzle, or a method of sequentially synthesizing nucleotides on the solid phase] to prepare an array such as a chip and measuring target nucleic acids through the use of hybridization using this array.

The kit or the device of the present invention comprises nucleic acids capable of specifically binding to the polynucleotides of at least one, preferably at least two, more preferably at least three, most preferably at least five to all of the ovarian tumor marker miRNAs, respectively, of the group A described above, or to a complementary strand(s) of the polynucleotide(s). The kit or the device of the present invention can optionally further comprise nucleic acids capable of specifically binding to the polynucleotides of at least one, preferably at least two, more preferably at least three, most preferably at least five to all of the ovarian tumor marker miRNAs, respectively, of the group B described above, or to a complementary strand(s) of the polynucleotide(s).

The kit or the device of the present invention can be used for detecting ovarian tumor as described in Section 4 below.

### 4. Method for Detecting Ovarian Tumor

The present invention further provides a method for detecting ovarian tumor, comprising using the above-mentioned nucleic acid(s) that can be used in the present invention (alternatively, e.g., the kit or the device of the present invention as described in Section 3 above) to measure: one or more expression levels of ovarian tumor-derived genes represented by: hsa-miR-4675, hsa-miR-4783-3p, hsa-miR-1228-5p, hsa-miR-4532, hsa-miR-6802-5p, hsa-miR-6784-5p, hsa-miR-3940-5p, hsa-miR-1307-3p, hsa-miR-8073, hsa-miR-3184-5p, hsa-miR-1233-5p, hsa-miR-6088, hsa-miR-5195-3p, hsa-miR-320b, hsa-miR-4649-5p, hsa-miR-6800-5p, hsa-miR-1343-3p, hsa-miR-4730, hsa-miR-6885-5p, hsa-miR-5100, hsa-miR-1203, hsa-miR-6756-5p, hsa-miR-373-5p, hsa-miR-1268a, hsa-miR-1260b, hsa-miR-4258, hsa-miR-4697-5p, hsa-miR-1469, hsa-miR-4515, hsa-miR-6861-5p, hsa-miR-6821-5p, hsa-miR-575, hsa-miR-6805-5p, hsa-miR-4758-5p, hsa-miR-3663-3p, hsa-miR-4530, hsa-miR-6798-5p, hsa-miR-6781-5p, hsa-miR-885-3p, hsa-miR-1273g-3p, hsa-miR-4787-3p, hsa-miR-4454, hsa-miR-4706, hsa-miR-1249-3p, hsa-miR-887-3p, hsa-miR-6786-5p, hsa-miR-1238-5p, hsa-miR-6749-5p, hsa-miR-6729-5p, hsa-miR-6825-5p, hsa-miR-663b, hsa-miR-6858-5p, hsa-miR-4690-5p, hsa-miR-6765-5p, hsa-miR-4710, hsa-miR-6775-5p, hsa-miR-371a-5p, hsa-miR-6816-5p, hsa-miR-296-3p, hsa-miR-7977, hsa-miR-8069, hsa-miR-6515-3p, hsa-miR-4687-5p, hsa-miR-1343-5p, hsa-miR-7110-5p, hsa-miR-4525, hsa-miR-3158-5p, hsa-miR-6787-5p, hsa-miR-614, hsa-miR-4689, hsa-miR-1185-2-3p, hsa-miR-1268b, hsa-miR-1228-3p, hsa-miR-1185-1-3p, hsa-miR-940, hsa-miR-939-5p, hsa-miR-6757-5p, hsa-miR-1275, hsa-miR-5001-5p, hsa-miR-6826-5p, hsa-miR-6765-3p, hsa-miR-3679-3p, hsa-miR-4718, hsa-miR-4286, hsa-miR-8059, hsa-miR-4447, hsa-miR-4448, hsa-miR-658, hsa-miR-6766-3p, hsa-miR-197-5p, hsa-miR-6887-5p, hsa-miR-6742-5p, hsa-miR-6729-3p, hsa-miR-5090, hsa-miR-7975, hsa-miR-4505, hsa-miR-6889-5p, hsa-miR-4708-3p, hsa-miR-6131, hsa-miR-1225-3p, hsa-miR-6132, hsa-miR-4734, hsa-miR-3194-3p, hsa-miR-638, hsa-miR-2467-3p, hsa-miR-4728-5p, hsa-miR-5572, hsa-miR-6789-5p, hsa-miR-8063, hsa-miR-4429, hsa-miR-6840-3p, hsa-miR-4476, hsa-miR-675-5p, hsa-miR-711, hsa-miR-6875-5p, hsa-miR-3160-5p, hsa-miR-1908-5p, hsa-miR-6726-5p, hsa-miR-1913, hsa-miR-8071, hsa-miR-3648, hsa-miR-4732-5p, hsa-miR-4787-5p, hsa-miR-3917, hsa-miR-619-5p, hsa-miR-1231, hsa-miR-342-5p, hsa-miR-4433a-5p, hsa-miR-6766-5p, hsa-miR-4707-5p, hsa-miR-7114-5p, hsa-miR-6872-3p, hsa-miR-6780b-5p, hsa-miR-7845-5p, hsa-miR-6798-3p, hsa-miR-665, hsa-miR-6848-5p, hsa-miR-5008-5p, hsa-miR-4294, hsa-miR-6511a-5p, hsa-miR-4435, hsa-miR-4747-3p, hsa-miR-6880-3p, hsa-miR-6869-5p, hsa-miR-7150, hsa-miR-1260a, hsa-miR-6877-5p, hsa-miR-6721-5p, hsa-miR-4656, hsa-miR-1229-5p, hsa-miR-4433a-3p, hsa-miR-4274, hsa-miR-4419b, hsa-miR-4674, hsa-miR-6893-5p, hsa-miR-6763-3p, hsa-miR-6762-5p, hsa-miR-6738-5p, hsa-miR-4513, hsa-miR-6746-5p, hsa-miR-6880-5p, hsa-miR-4736, hsa-miR-718, hsa-miR-6717-5p, hsa-miR-7847-3p, hsa-miR-760, hsa-miR-1199-5p, hsa-miR-6813-5p, hsa-miR-6769a-5p, hsa-miR-1193, hsa-miR-7108-3p, hsa-miR-6741-5p, hsa-miR-4298, hsa-miR-6796-3p, hsa-miR-4750-5p, hsa-miR-6785-5p, hsa-miR-1292-3p, hsa-miR-4749-3p, hsa-miR-6800-3p, hsa-miR-4722-5p, hsa-miR-4746-3p, hsa-miR-4450, hsa-miR-6795-5p, hsa-miR-365a-5p, hsa-miR-498, hsa-miR-6797-5p, hsa-miR-1470, hsa-miR-6851-5p, hsa-miR-1247-3p, hsa-miR-5196-5p, hsa-miR-208a-5p, hsa-miR-6842-5p, hsa-miR-150-3p, hsa-miR-4534, hsa-miR-3135b, hsa-miR-3131, hsa-miR-4792, hsa-miR-6510-5p, hsa-miR-504-3p, hsa-miR-3619-3p, hsa-miR-671-5p, hsa-miR-4667-5p, hsa-miR-4430, hsa-miR-3195, hsa-miR-3679-5p, hsa-miR-6076, hsa-miR-6515-5p, hsa-miR-6820-5p, hsa-miR-4634, hsa-miR-187-5p, hsa-miR-6763-5p, hsa-miR-1908-3p, hsa-miR-1181, hsa-miR-6782-5p, hsa-miR-5010-5p, hsa-miR-6870-5p, hsa-miR-6124, hsa-miR-1249-5p, hsa-miR-6511b-5p, hsa-miR-1254, hsa-miR-4727-3p, hsa-miR-4259, hsa-miR-4771, hsa-miR-3622a-5p, hsa-miR-4480, hsa-miR-4740-5p, hsa-miR-6777-5p, hsa-miR-6794-5p, hsa-miR-4687-3p, hsa-miR-6743-5p, hsa-miR-6771-5p, hsa-miR-3141, hsa-miR-3162-5p, hsa-miR-4271, hsa-miR-1227-5p, hsa-miR-4257, hsa-miR-4270, hsa-miR-4516, hsa-miR-4651, hsa-miR-4725-3p, hsa-miR-6125, hsa-miR-6732-5p, hsa-miR-6791-5p, hsa-miR-6819-5p, hsa-miR-6891-5p, hsa-miR-7108-5p, hsa-miR-7109-5p, hsa-miR-642b-3p, and hsa-miR-642a-3p; and optionally one or more expression levels of ovarian tumor-derived genes represented by: hsa-miR-320a, hsa-miR-663a, hsa-miR-328-5p, hsa-miR-128-2-5p, hsa-miR-125a-3p, hsa-miR-191-5p, hsa-miR-92b-5p, hsa-miR-296-5p, hsa-miR-1246, hsa-miR-92a-2-5p, hsa-miR-128-1-5p, hsa-miR-1290, hsa-miR-211-3p, hsa-miR-744-5p, hsa-miR-135a-3p, hsa-miR-451a, hsa-miR-625-3p, hsa-miR-92a-3p, hsa-miR-422a, hsa-miR-483-5p, hsa-miR-652-5p, hsa-miR-24-3p, hsa-miR-23b-3p, hsa-miR-23a-3p, hsa-miR-92b-3p, hsa-miR-22-3p, in a sample in vitro (e.g., expression profiles), and evaluating in vitro whether the subject has ovarian tumor or not with the expression levels measured (and control expression levels of healthy subjects optionally measured in the same way as above). In the method, for example, the expression levels of the genes in the sample, such as blood, serum, or plasma, collected from a subject suspected of having ovarian tumor and subjects without ovarian tumor, and control expression levels in the samples collected from subjects without ovarian tumor can be used (e.g., for comparing both expression levels) to evaluate the subject as having ovarian tumor when the expression level(s) of the target nucleic acid(s) is different between the samples.

This method of the present invention enables a limitedly invasive, early diagnosis of the cancer with high sensitivity and high specificity and thereby brings about early treatment and improved prognosis. In addition, exacerbation of the disease or the effectiveness of surgical, radiotherapeutic, and chemotherapeutic treatments can be monitored by the present invention.

The method for extracting the ovarian tumor-derived gene(s) from the sample such as blood, serum, or plasma according to the present invention is particularly preferably prepared by the addition of a reagent for RNA extraction in 3D-Gene(™) RNA extraction reagent from liquid sample kit (Toray Industries, Inc., Japan). A general acidic phenol method (acid guanidinium-phenol-chloroform (AGPC)) may be used, or Trizol(™) (Life Technologies Corp.) may be used. The ovarian tumor-derived gene(s) may be prepared by the addition of a reagent for RNA extraction containing acidic phenol, such as Trizol (Life Technologies Corp.) or Isogen (Nippon Gene Co., Ltd., Japan). Alternatively, a kit such as miRNeasy(™) Mini Kit (Qiagen N.V.) may be used, though the method is not limited thereto.

The present invention also provides use of the kit or the device of the present invention for detecting in vitro an expression product(s) of an ovarian tumor-derived miRNA gene(s) in a sample from a subject.

In the method of the present invention, the kit or the device described above comprises a single polynucleotide or any possible combination of polynucleotides that can be used in the present invention as described above.

In the detection or (genetic) diagnosis of ovarian tumor according to the present invention, each polynucleotide contained in the kit or the device of the present invention can be used as a probe or a primer. In the case of using the polynucleotides as primers, TaqMan(™) MicroRNA Assays from Life Technologies Corp., miScript PCR System from Qiagen N.V., or the like can be used, though the method is not limited thereto.

In the method of the present invention, measurement of the gene expression levels can be performed using the above-mentioned primers or probes according to a routine method in a method known in the art for specifically detecting the particular genes, for example, a hybridization technique such as Northern blot, Southern blot, in situ hybridization, Northern hybridization, or Southern hybridization, a quantitative amplification technique such as quantitative RT-PCR, or a method with a next-generation sequencer. A body fluid such as blood, serum, plasma, or urine from a subject is collected as a sample to be assayed according to the type of the detection method used. Alternatively, total RNA prepared from such a body fluid by the method described above may be used, and various polynucleotides including cDNA prepared on the basis of the RNA may be used.

The method, the kit or the device of the present invention is useful for the diagnosis of ovarian tumor or the detection of the presence or absence of ovarian tumor. Specifically, the detection of ovarian tumor using the method, the kit or the device can be performed by detecting in vitro an expression level(s) of a gene(s) which is detected by the method or detected using the nucleic acid probe(s) or the primer(s) contained in the kit or the device, in a sample such as blood, serum, plasma, or urine from a subject suspected of having ovarian tumor. The subject suspected of having ovarian tumor can be evaluated as having ovarian tumor when the expression level(s) of a polynucleotide(s) consisting of a nucleotide sequence(s) represented by at least one of, for example, SEQ ID NOs: 1 to 247, 251, and 268 and optionally a nucleotide sequence(s) represented by one or more of, for example, SEQ ID NOs: 248 to 250, 252 to 267, and 269 to 275, as target nucleic acids, in the sample such as blood, serum, plasma, or urine of the subject, is statistically significantly high compared to an expression level(s) thereof in the sample such as blood, serum, or plasma, or urine of a subject without ovarian tumor (i.e., also referred to as a control animal).

In the method of the present invention, or the method using the kit or the device of the present invention, the method for detecting the absence of ovarian tumor or the presence of ovarian tumor in a sample from a subject comprises collecting a body fluid such as blood, serum, plasma, or urine of a subject, and measuring the expression level(s) of the target gene(s) (or target nucleic acid(s)) contained therein using one or more polynucleotides (including a variant(s), a fragment(s), or a derivative(s)) selected from the groups of polynucleotides of the present invention, to evaluate the presence or absence of ovarian tumor or to detect ovarian tumor. The method for detecting ovarian tumor according to the present invention can also be used to evaluate or diagnose, for example, the presence or absence of amelioration of the disease or the degree of amelioration thereof in a ovarian tumor patient in the case that an ovarian cancer-related therapeutic drug which is known in the art or on a development stage (including cisplatin, cyclophosphamide, doxorubicin, etoposide, carboplatin, paclitaxel, and combination drugs thereof as non-limiting examples) is administered to the patient for treatment or amelioration of the disease.

The method of the present invention can comprise, for example, the following steps (a), (b), and (c):
(a) a step of contacting in vitro a sample from a subject with a polynucleotide(s) contained in the kit or the device of the present invention;
(b) a step of measuring an expression level(s) of the target nucleic acid(s) in the sample using the polynucleotide(s) as a nucleic acid probe(s) or primer(s); and
(c) a step of evaluating the presence or absence of ovarian tumor (cells) in the subject on the basis of the measurement results in the step (b).

In one embodiment, the present invention provides a method for detecting ovarian tumor, comprising: measuring an expression level(s) of a target nucleic acid(s) in a sample of a subject using a nucleic acid(s) capable of specifically binding to at least one, preferably at least two polynucleotides selected from the following miRNAs: miR-4675, miR-4783-3p, miR-1228-5p, miR-4532, miR-6802-5p, miR-6784-5p, miR-3940-5p, miR-1307-3p, miR-8073, miR-3184-5p, miR-1233-5p, miR-6088, miR-5195-3p, miR-320b, miR-4649-5p, miR-6800-5p, miR-1343-3p, miR-4730, miR-6885-5p, miR-5100, miR-1203, miR-6756-5p, miR-373-5p, miR-1268a, miR-1260b, miR-4258, miR-4697-5p, miR-1469, miR-4515, miR-6861-5p, miR-6821-5p, miR-575, miR-6805-5p, miR-4758-5p, miR-3663-3p, miR-4530, miR-6798-5p, miR-6781-5p, miR-885-3p, miR-1273g-3p, miR-4787-3p, miR-4454, miR-4706, miR-1249-3p, miR-887-3p, miR-6786-5p, miR-1238-5p, miR-6749-5p, miR-6729-5p, miR-6825-5p, miR-663b, miR-6858-5p, miR-4690-5p, miR-6765-5p, miR-4710, miR-6775-5p, miR-371a-5p, miR-6816-5p, miR-296-3p, miR-7977, miR-8069, miR-6515-3p, miR-4687-5p, miR-1343-5p, miR-7110-5p, miR-4525, miR-3158-5p, miR-6787-5p, miR-614, miR-4689, miR-1185-2-3p, miR-1268b, miR-1228-3p, miR-1185-1-3p, miR-940, miR-939-5p, miR-6757-5p, miR-1275, miR-5001-5p, miR-6826-5p, miR-6765-3p, miR-3679-3p, miR-4718, miR-4286, miR-8059, miR-4447, miR-4448, miR-658, miR-6766-3p, miR-197-5p, miR-6887-5p, miR-6742-5p, miR-6729-3p, miR-5090, miR-7975, miR-4505, miR-6889-5p, miR-4708-3p, miR-6131, miR-1225-3p, miR-6132, miR-4734, miR-3194-3p, miR-638, miR-2467-3p, miR-4728-5p, miR-5572, miR-6789-5p, miR-8063, miR-4429, miR-6840-3p, miR-4476, miR-675-5p, miR-711, miR-6875-5p, miR-3160-5p, miR-1908-5p, miR-6726-5p, miR-1913, miR-8071, miR-3648, miR-4732-5p, miR-4787-5p, miR-3917, miR-619-5p, miR-1231, miR-342-5p, miR-4433a-5p, miR-6766-5p, miR-4707-5p, miR-7114-5p, miR-6872-3p, miR-6780b-5p, miR-7845-5p, miR-6798-3p, miR-665, miR-6848-5p, miR-5008-5p, miR-4294, miR-6511a-5p, miR-4435, miR-4747-3p, miR-6880-3p, miR-6869-5p, miR-7150, miR-1260a, miR-6877-5p, miR-6721-5p, miR-4656, miR-1229-5p, miR-4433a-3p, miR-4274, miR-4419b, miR-4674, miR-6893-5p, miR-6763-3p, miR-6762-5p, miR-6738-5p, miR-4513, miR-6746-5p, miR-6880-5p, miR-4736, miR-718, miR-6717-5p, miR-7847-3p, miR-760, miR-1199-5p, miR-6813-5p, miR-6769a-5p, miR-1193, miR-7108-3p, miR-6741-5p, miR-4298, miR-6796-3p, miR-4750-5p, miR-6785-5p, miR-1292-3p, miR-4749-3p, miR-6800-3p, miR-4722-5p, miR-4746-3p, miR-4450, miR-6795-5p, miR-365a-5p, miR-498, miR-6797-5p, miR-1470, miR-6851-5p, miR-1247-3p, miR-5196-5p, miR-208a-5p, miR-6842-5p, miR-150-3p, miR-4534, miR-3135b, miR-3131, miR-4792, miR-6510-5p, miR-504-3p, miR-3619-3p, miR-671-5p, miR-4667-5p, miR-4430, miR-3195, miR-3679-5p, miR-6076, miR-6515-5p, miR-6820-5p, miR-4634, miR-187-5p, miR-6763-5p, miR-1908-3p, miR-1181, miR-6782-5p, miR-5010-5p, miR-6870-5p, miR-6124, miR-1249-5p, miR-6511b-5p, miR-1254, miR-4727-3p, miR-4259, miR-4771, miR-3622a-5p, miR-4480, miR-4740-5p, miR-6777-5p, miR-6794-5p, miR-4687-3p, miR-6743-5p, miR-6771-5p, miR-3141, miR-3162-5p, miR-4271, miR-1227-5p, miR-4257, miR-4270, miR-4516, miR-4651, miR-4725-3p, miR-6125, miR-6732-5p, miR-6791-5p, miR-6819-5p, miR-6891-5p, miR-7108-5p, miR-7109-5p, miR-642b-3p, and miR-642a-3p, or to a complementary strand(s) of the polynucleotide(s); or a nucleic acid(s) for detecting the polynucleotide(s); and evaluating in vitro whether or not the subject has ovarian tumor using the above-measured expression levels and control expression levels of a healthy subject(s) (see the definition described above) measured in the same way as above.

As used herein, the term “evaluating” is evaluation support based on results of in vitro examination, not physician's judgment.

As described above, in the method of the present invention, specifically, miR-4675 is hsa-miR-4675, miR-4783-3p is hsa-miR-4783-3p, miR-1228-5p is hsa-miR-1228-5p, miR-4532 is hsa-miR-4532, miR-6802-5p is hsa-miR-6802-5p, miR-6784-5p is hsa-miR-6784-5p, miR-3940-5p is hsa-miR-3940-5p, miR-1307-3p is hsa-miR-1307-3p, miR-8073 is hsa-miR-8073, miR-3184-5p is hsa-miR-3184-5p, miR-1233-5p is hsa-miR-1233-5p, miR-6088 is hsa-miR-6088, miR-5195-3p is hsa-miR-5195-3p, miR-320b is hsa-miR-320b, miR-4649-5p is hsa-miR-4649-5p, miR-6800-5p is hsa-miR-6800-5p, miR-1343-3p is hsa-miR-1343-3p, miR-4730 is hsa-miR-4730, miR-6885-5p is hsa-miR-6885-5p, miR-5100 is hsa-miR-5100, miR-1203 is hsa-miR-1203, miR-6756-5p is hsa-miR-6756-5p, miR-373-5p is hsa-miR-373-5p, miR-1268a is hsa-miR-1268a, miR-1260b is hsa-miR-1260b, miR-4258 is hsa-miR-4258, miR-4697-5p is hsa-miR-4697-5p, miR-1469 is hsa-miR-1469, miR-4515 is hsa-miR-4515, miR-6861-5p is hsa-miR-6861-5p, miR-6821-5p is hsa-miR-6821-5p, miR-575 is hsa-miR-575, miR-6805-5p is hsa-miR-6805-5p, miR-4758-5p is hsa-miR-4758-5p, miR-3663-3p is hsa-miR-3663-3p, miR-4530 is hsa-miR-4530, miR-6798-5p is hsa-miR-6798-5p, miR-6781-5p is hsa-miR-6781-5p, miR-885-3p is hsa-miR-885-3p, miR-1273g-3p is hsa-miR-1273g-3p, miR-4787-3p is hsa-miR-4787-3p, miR-4454 is hsa-miR-4454, miR-4706 is hsa-miR-4706, miR-1249-3p is hsa-miR-1249-3p, miR-887-3p is hsa-miR-887-3p, miR-6786-5p is hsa-miR-6786-5p, miR-1238-5p is hsa-miR-1238-5p, miR-6749-5p is hsa-miR-6749-5p, miR-6729-5p is hsa-miR-6729-5p, miR-6825-5p is hsa-miR-6825-5p, miR-663b is hsa-miR-663b, miR-6858-5p is hsa-miR-6858-5p, miR-4690-5p is hsa-miR-4690-5p, miR-6765-5p is hsa-miR-6765-5p, miR-4710 is hsa-miR-4710, miR-6775-5p is hsa-miR-6775-5p, miR-371a-5p is hsa-miR-371a-5p, miR-6816-5p is hsa-miR-6816-5p, miR-296-3p is hsa-miR-296-3p, miR-7977 is hsa-miR-7977, miR-8069 is hsa-miR-8069, miR-6515-3p is hsa-miR-6515-3p, miR-4687-5p is hsa-miR-4687-5p, miR-1343-5p is hsa-miR-1343-5p, miR-7110-5p is hsa-miR-7110-5p, miR-4525 is hsa-miR-4525, miR-3158-5p is hsa-miR-3158-5p, miR-6787-5p is hsa-miR-6787-5p, miR-614 is hsa-miR-614, miR-4689 is hsa-miR-4689, miR-1185-2-3p is hsa-miR-1185-2-3p, miR-1268b is hsa-miR-1268b, miR-1228-3p is hsa-miR-1228-3p, miR-1185-1-3p is hsa-miR-1185-1-3p, miR-940 is hsa-miR-940, miR-939-5p is hsa-miR-939-5p, miR-6757-5p is hsa-miR-6757-5p, miR-1275 is hsa-miR-1275, miR-5001-5p is hsa-miR-5001-5p, miR-6826-5p is hsa-miR-6826-5p, miR-6765-3p is hsa-miR-6765-3p, miR-3679-3p is hsa-miR-3679-3p, miR-4718 is hsa-miR-4718, miR-4286 is hsa-miR-4286, miR-8059 is hsa-miR-8059, miR-4447 is hsa-miR-4447, miR-4448 is hsa-miR-4448, miR-658 is hsa-miR-658, miR-6766-3p is hsa-miR-6766-3p, miR-197-5p is hsa-miR-197-5p, miR-6887-5p is hsa-miR-6887-5p, miR-6742-5p is hsa-miR-6742-5p, miR-6729-3p is hsa-miR-6729-3p, miR-5090 is hsa-miR-5090, miR-7975 is hsa-miR-7975, miR-4505 is hsa-miR-4505, miR-6889-5p is hsa-miR-6889-5p, miR-4708-3p is hsa-miR-4708-3p, miR-6131 is hsa-miR-6131, miR-1225-3p is hsa-miR-1225-3p, miR-6132 is hsa-miR-6132, miR-4734 is hsa-miR-4734, miR-3194-3p is hsa-miR-3194-3p, miR-638 is hsa-miR-638, miR-2467-3p is hsa-miR-2467-3p, miR-4728-5p is hsa-miR-4728-5p, miR-5572 is hsa-miR-5572, miR-6789-5p is hsa-miR-6789-5p, miR-8063 is hsa-miR-8063, miR-4429 is hsa-miR-4429, miR-6840-3p is hsa-miR-6840-3p, miR-4476 is hsa-miR-4476, miR-675-5p is hsa-miR-675-5p, miR-711 is hsa-miR-711, miR-6875-5p is hsa-miR-6875-5p, miR-3160-5p is hsa-miR-3160-5p, miR-1908-5p is hsa-miR-1908-5p, miR-6726-5p is hsa-miR-6726-5p, miR-1913 is hsa-miR-1913, miR-8071 is hsa-miR-8071, miR-3648 is hsa-miR-3648, miR-4732-5p is hsa-miR-4732-5p, miR-4787-5p is hsa-miR-4787-5p, miR-3917 is hsa-miR-3917, miR-619-5p is hsa-miR-619-5p, miR-1231 is hsa-miR-1231, miR-342-5p is hsa-miR-342-5p, miR-4433a-5p is hsa-miR-4433a-5p, miR-6766-5p is hsa-miR-6766-5p, miR-4707-5p is hsa-miR-4707-5p, miR-7114-5p is hsa-miR-7114-5p, miR-6872-3p is hsa-miR-6872-3p, miR-6780b-5p is hsa-miR-6780b-5p, miR-7845-5p is hsa-miR-7845-5p, miR-6798-3p is hsa-miR-6798-3p, miR-665 is hsa-miR-665, miR-6848-5p is hsa-miR-6848-5p, miR-5008-5p is hsa-miR-5008-5p, miR-4294 is hsa-miR-4294, miR-6511a-5p is hsa-miR-6511a-5p, miR-4435 is hsa-miR-4435, miR-4747-3p is hsa-miR-4747-3p, miR-6880-3p is hsa-miR-6880-3p, miR-6869-5p is hsa-miR-6869-5p, miR-7150 is hsa-miR-7150, miR-1260a is hsa-miR-1260a, miR-6877-5p is hsa-miR-6877-5p, miR-6721-5p is hsa-miR-6721-5p, miR-4656 is hsa-miR-4656, miR-1229-5p is hsa-miR-1229-5p, miR-4433a-3p is hsa-miR-4433a-3p, miR-4274 is hsa-miR-4274, miR-4419b is hsa-miR-4419b, miR-4674 is hsa-miR-4674, miR-6893-5p is hsa-miR-6893-5p, miR-6763-3p is hsa-miR-6763-3p, miR-6762-5p is hsa-miR-6762-5p, miR-6738-5p is hsa-miR-6738-5p, miR-4513 is hsa-miR-4513, miR-6746-5p is hsa-miR-6746-5p, miR-6880-5p is hsa-miR-6880-5p, miR-4736 is hsa-miR-4736, miR-718 is hsa-miR-718, miR-6717-5p is hsa-miR-6717-5p, miR-7847-3p is hsa-miR-7847-3p, miR-760 is hsa-miR-760, miR-1199-5p is hsa-miR-1199-5p, miR-6813-5p is hsa-miR-6813-5p, miR-6769a-5p is hsa-miR-6769a-5p, miR-1193 is hsa-miR-1193, miR-7108-3p is hsa-miR-7108-3p, miR-6741-5p is hsa-miR-6741-5p, miR-4298 is hsa-miR-4298, miR-6796-3p is hsa-miR-6796-3p, miR-4750-5p is hsa-miR-4750-5p, miR-6785-5p is hsa-miR-6785-5p, miR-1292-3p is hsa-miR-1292-3p, miR-4749-3p is hsa-miR-4749-3p, miR-6800-3p is hsa-miR-6800-3p, miR-4722-5p is hsa-miR-4722-5p, miR-4746-3p is hsa-miR-4746-3p, miR-4450 is hsa-miR-4450, miR-6795-5p is hsa-miR-6795-5p, miR-365a-5p is hsa-miR-365a-5p, miR-498 is hsa-miR-498, miR-6797-5p is hsa-miR-6797-5p, miR-1470 is hsa-miR-1470, miR-6851-5p is hsa-miR-6851-5p, miR-1247-3p is hsa-miR-1247-3p, miR-5196-5p is hsa-miR-5196-5p, miR-208a-5p is hsa-miR-208a-5p, miR-6842-5p is hsa-miR-6842-5p, miR-150-3p is hsa-miR-150-3p, miR-4534 is hsa-miR-4534, miR-3135b is hsa-miR-3135b, miR-3131 is hsa-miR-3131, miR-4792 is hsa-miR-4792, miR-6510-5p is hsa-miR-6510-5p, miR-504-3p is hsa-miR-504-3p, miR-3619-3p is hsa-miR-3619-3p, miR-671-5p is hsa-miR-671-5p, miR-4667-5p is hsa-miR-4667-5p, miR-4430 is hsa-miR-4430, miR-3195 is hsa-miR-3195, miR-3679-5p is hsa-miR-3679-5p, miR-6076 is hsa-miR-6076, miR-6515-5p is hsa-miR-6515-5p, miR-6820-5p is hsa-miR-6820-5p, miR-4634 is hsa-miR-4634, miR-187-5p is hsa-miR-187-5p, miR-6763-5p is hsa-miR-6763-5p, miR-1908-3p is hsa-miR-1908-3p, miR-1181 is hsa-miR-1181, miR-6782-5p is hsa-miR-6782-5p, miR-5010-5p is hsa-miR-5010-5p, miR-6870-5p is hsa-miR-6870-5p, miR-6124 is hsa-miR-6124, miR-1249-5p is hsa-miR-1249-5p, miR-6511b-5p is hsa-miR-6511b-5p, miR-1254 is hsa-miR-1254, miR-4727-3p is hsa-miR-4727-3p, miR-4259 is hsa-miR-4259, miR-4771 is hsa-miR-4771, miR-3622a-5p is hsa-miR-3622a-5p, miR-4480 is hsa-miR-4480, miR-4740-5p is hsa-miR-4740-5p, miR-6777-5p is hsa-miR-6777-5p, miR-6794-5p is hsa-miR-6794-5p, miR-4687-3p is hsa-miR-4687-3p, miR-6743-5p is hsa-miR-6743-5p, miR-6771-5p is hsa-miR-6771-5p, miR-3141 is hsa-miR-3141, miR-3162-5p is hsa-miR-3162-5p, miR-4271 is hsa-miR-4271, miR-1227-5p is hsa-miR-1227-5p, miR-4257 is hsa-miR-4257, miR-4270 is hsa-miR-4270, miR-4516 is hsa-miR-4516, miR-4651 is hsa-miR-4651, miR-4725-3p is hsa-miR-4725-3p, miR-6125 is hsa-miR-6125, miR-6732-5p is hsa-miR-6732-5p, miR-6791-5p is hsa-miR-6791-5p, miR-6819-5p is hsa-miR-6819-5p, miR-6891-5p is hsa-miR-6891-5p, miR-7108-5p is hsa-miR-7108-5p, miR-7109-5p is hsa-miR-7109-5p, miR-642b-3p is hsa-miR-642b-3p, and miR-642a-3p is hsa-miR-642a-3p.

Additionally, in one embodiment, the nucleic acid(s) (e.g., a probe(s) or a primer(s)) in the method of the present invention is selected from the group consisting of, for example, the following polynucleotides (a) to (e):
(a) a polynucleotide consisting of a nucleotide sequence represented by any of SEQ ID NOs: 1 to 247, 251 and 268 or a nucleotide sequence derived from the nucleotide sequence by the replacement of u with t, a variant thereof, a derivative thereof, or a fragment thereof comprising 15 or more consecutive nucleotides;
(b) a polynucleotide comprising a nucleotide sequence represented by any of SEQ ID NOs: 1 to 247, 251 and 268;
(c) a polynucleotide consisting of a nucleotide sequence complementary to a nucleotide sequence represented by any of SEQ ID NOs: 1 to 247, 251 and 268 or a nucleotide sequence derived from the nucleotide sequence by the replacement of u with t, a variant thereof, a derivative thereof, or a fragment thereof comprising 15 or more consecutive nucleotides;
(d) a polynucleotide comprising a nucleotide sequence complementary to a nucleotide sequence represented by any of SEQ ID NOs: 1 to 247, 251 and 268 or a nucleotide sequence derived from the nucleotide sequence by the replacement of u with t; and
(e) a polynucleotide hybridizing under stringent conditions to any of the polynucleotides (a) to (d).

The nucleic acid(s) used in the method of the present invention can further comprise a nucleic acid(s) capable of specifically binding to at least one polynucleotide selected from the following miRNAs: miR-320a, miR-663a, miR-328-5p, miR-128-2-5p, miR-125a-3p, miR-191-5p, miR-92b-5p, miR-296-5p, miR-1246, miR-92a-2-5p, miR-128-1-5p, miR-1290, miR-211-3p, miR-744-5p, miR-135a-3p, miR-451a, miR-625-3p, miR-92a-3p, miR-422a, miR-483-5p, miR-652-5p, miR-24-3p, miR-23b-3p, miR-23a-3p, miR-92b-3p, miR-22-3p, or to a complementary strand of the polynucleotide.

Specifically, miR-320a is hsa-miR-320a, miR-663a is hsa-miR-663a, miR-328-5p is hsa-miR-328-5p, miR-128-2-5p is hsa-miR-128-2-5p, miR-125a-3p is hsa-miR-125a-3p, miR-191-5p is hsa-miR-191-5p, miR-92b-5p is hsa-miR-92b-5p, miR-296-5p is hsa-miR-296-5p, miR-1246 is hsa-miR-1246, miR-92a-2-5p is hsa-miR-92a-2-5p, miR-128-1-5p is hsa-miR-128-1-5p, miR-1290 is hsa-miR-1290, miR-211-3p is hsa-miR-211-3p, miR-744-5p is hsa-miR-744-5p, miR-135a-3p is hsa-miR-135a-3p, miR-451a is hsa-miR-451a, miR-625-3p is hsa-miR-625-3p, miR-92a-3p is hsa-miR-92a-3p, miR-422a is hsa-miR-422a, miR-483-5p is hsa-miR-483-5p, miR-652-5p is hsa-miR-652-5p, miR-24-3p is hsa-miR-24-3p, miR-23b-3p is hsa-miR-23b-3p, miR-23a-3p is hsa-miR-23a-3p, miR-92b-3p is hsa-miR-92b-3p, and miR-22-3p is hsa-miR-22-3.

In one embodiment, the nucleic acid(s) may further be selected from, for example, the group consisting of the following polynucleotides (f) to j):
(f) a polynucleotide consisting of a nucleotide sequence represented by any of SEQ ID NOs: 248 to 250, 252 to 267, and 269 to 275 or a nucleotide sequence derived from the nucleotide sequence by the replacement of u with t, a variant thereof, a derivative thereof, or a fragment thereof comprising 15 or more consecutive nucleotides;
(g) a polynucleotide comprising a nucleotide sequence represented by any of SEQ ID NOs: 248 to 250, 252 to 267, and 269 to 275;
(h) a polynucleotide consisting of a nucleotide sequence complementary to a nucleotide sequence represented by any of SEQ ID NOs: 248 to 250, 252 to 267, and 269 to 275 or a nucleotide sequence derived from the nucleotide sequence by the replacement of u with t, a variant thereof, a derivative thereof, or a fragment thereof comprising 15 or more consecutive nucleotides;
(i) a polynucleotide comprising a nucleotide sequence complementary to a nucleotide sequence represented by any of SEQ ID NOs: 248 to 250, 252 to 267, and 269 to 275 or a nucleotide sequence derived from the nucleotide sequence by the replacement of u with t; and
j) a polynucleotide hybridizing under stringent conditions to any of the polynucleotides (f) to (i).

Examples of the sample used in the method of the present invention can include samples prepared from living tissues (preferably ovarian tissues or fallopian tube tissues) or body fluids such as blood, serum, plasma, and urine from subjects. Specifically, for example, an RNA-containing sample prepared from the tissue, a polynucleotide-containing sample further prepared therefrom, a body fluid such as blood, serum, plasma, or urine, a portion or the whole of a living tissue collected from the subject by biopsy or the like, or a living tissue excised by surgery can be used, and the sample for measurement can be prepared therefrom.

As used herein, the subject refers to a mammal, for example, a human, a monkey, a mouse, or a rat, without any limitation, and is preferably a human.

The steps of the method of the present invention can be changed according to the type of the sample to be measured.

In the case of using RNA as an analyte, the method for detecting ovarian tumor (cells) can comprise, for example, the following steps (a), (b), and (c):
(a) a step of binding RNA prepared from a sample from a subject (wherein, for example, the 3′ end of the RNA may be polyadenylated for quantitative RT-PCR in step
(b)) or complementary polynucleotides (cDNAs) transcribed from the RNA to a polynucleotide(s) in the kit of the present invention;
(b) a step of measuring the sample-derived RNA or the cDNAs synthesized from the RNA, which is/are bound to the polynucleotide(s), by hybridization using the polynucleotide(s) as a nucleic acid probe(s) or by quantitative RT-PCR using the polynucleotide(s) as a primer(s); and
(c) a step of evaluating the presence or absence of ovarian tumor (or ovarian tumor-derived gene) on the basis of the measurement results of the step (b).

For example, various hybridization methods can be used for measuring the expression level(s) of a target gene(s), or detecting, examining, evaluating, or diagnosing ovarian tumor (or ovarian tumor-derived gene) in vitro according to the present invention. For example, Northern blot, Southern blot, DNA chip analysis, in situ hybridization, Northern hybridization, or Southern hybridization can be used as such a hybridization method. PCR such as quantitative RT-PCR can also be used in combination with hybridization method, or as an alternative thereof.

In the case of using the Northern blot, the presence or absence of expression of each gene or the expression level thereof in the RNA can be detected or measured by use of, for example, the nucleic acid probe(s) that can be used in the present invention. Specific examples thereof can include a method which comprises labeling the nucleic acid probe (or a complementary strand) with a radioisotope (³²P, ³³P, ³⁵S, etc.), a fluorescent material, or the like, hybridizing the labeled product with the tissue-derived RNA from a subject, which is transferred to a nylon membrane or the like according to a routine method, and then detecting and measuring a signal derived from the label (radioisotope or fluorescent material) on the formed DNA/RNA duplex using a radiation detector (examples thereof can include BAS-1800 II (Fujifilm Corp.)) or a fluorescence detector (examples thereof can include STORM 865 (GE Healthcare Japan Corp.)).

In the case of using the quantitative RT-PCR, the presence or absence of expression of each gene or the expression level thereof in the RNA can be detected or measured by use of, for example, the primer that can be used in the present invention. Specific examples thereof can include a method which comprises recovering the tissue-derived RNA from a subject, preparing cDNAs according to reverse transcription using 3′-end polyadenylation treatment, specific primers, and the like, hybridizing a pair of primers (consisting of a plus strand and a reverse strand binding to the cDNA) prepared from the kit for detection of the present invention with the cDNA such that the region of each target gene marker can be amplified with the cDNA as a template, and performing PCR according to a routine method to detect the obtained single-stranded or double-stranded DNA. The method for detecting the single-stranded or double-stranded DNA can include a method of performing the PCR using the primers labeled in advance with a radioisotope or a fluorescent material, a method of electrophoresing the PCR product on an agarose gel and staining the double-stranded DNA with ethidium bromide or the like for detection, and a method of transferring the produced single-stranded or double-stranded DNA to a nylon membrane or the like according to a routine method and hybridizing the single-stranded or double-stranded DNA to a labeled nucleic acid probe for detection.

In the case of using the nucleic acid array analysis, an RNA chip or a DNA chip in which the kit or device for detection of the present invention is attached as nucleic acid probes (single-stranded or double-stranded) to a substrate (solid phase), for example, is used. Regions having the attached nucleic acid probes are referred to as probe spots, and regions having no attached nucleic acid probe are referred to as blank spots. A group of genes immobilized on a solid-phase substrate is generally called a nucleic acid chip, a nucleic acid array, a microarray, or the like. The DNA or RNA array includes a DNA or RNA macroarray and a DNA or RNA microarray. In the present specification, the term “chip” includes these arrays. 3D-Gene(™) Human miRNA Oligo chip (Toray Industries, Inc., Japan) can be used as the DNA chip, though the DNA chip is not limited thereto.

Examples of the measurement using the DNA chip can include, but are not limited to, a method of detecting and measuring a signal derived from the label on the kit or device for detection using an image detector (examples thereof can include Typhoon 9410 (GE Healthcare) and 3D-Gene(™) scanner (Toray Industries, Inc., Japan)).

The “stringent conditions” used herein are, as mentioned above, conditions under which a nucleic acid probe hybridizes to its target sequence to a detectably larger extent (e.g., a measurement value equal to or larger than “(a mean of background measurement values)+(a standard error of the background measurement values)×2)”) than that for other sequences.

The stringent conditions are defined by hybridization and subsequent washing. Examples of the hybridization conditions include, but not limited to, 30° C. to 60° C. for 1 to 24 hours in a solution containing SSC, a surfactant, formamide, dextran sulfate, a blocking agent(s), etc. In this context, 1×SSC is an aqueous solution (pH 7.0) containing 150 mM sodium chloride and 15 mM sodium citrate. The surfactant includes, for example, SDS (sodium dodecyl sulfate), Triton, or Tween. The hybridization conditions more preferably comprise 3-10×SSC and 0.1-1% SDS. Examples of the conditions for the washing, following the hybridization, which is another condition to define the stringent conditions, can include conditions comprising continuous washing at 30° C. in a solution containing 0.5×SSC and 0.1% SDS, at 30° C. in a solution containing 0.2×SSC and 0.1% SDS, and at 30° C. in a 0.05×SSC solution. It is desirable that the complementary strand should maintain its hybridized state with a target plus strand even by washing under such conditions. Specifically, examples of such a complementary strand can include a strand consisting of a nucleotide sequence in a completely complementary relationship with the nucleotide sequence of the target plus (+) strand, and a strand consisting of a nucleotide sequence having at least 80%, preferably at least 85%, more preferably at least 90% or at least 95% identity to the strand.

Other examples of the “stringent conditions” for the hybridization are described in, for example, Sambrook, J. & Russel, D., Molecular Cloning, A LABORATORY MANUAL, Cold Spring Harbor Laboratory Press, published on Jan. 15, 2001, Vol. 1, 7.42 to 7.45 and Vol. 2, 8.9 to 8.17, and can be used in the present invention.

Examples of the conditions for carrying out PCR using polynucleotide fragments in the kit of the present invention as primers include treatment for approximately 15 seconds to 1 minute at 5 to 10° C. plus a Tm value calculated from the sequences of the primers, using a PCR buffer having composition such as 10 mM Tris-HCL (pH 8.3), 50 mM KCL, and 1 to 2 mM MgCl₂. Examples of the method for calculating such a Tm value include Tm value=2×(the number of adenine residues+the number of thymine residues)+4×(the number of guanine residues+the number of cytosine residues).

In the case of using the quantitative RT-PCR, a commercially available kit for measurement specially designed for quantitatively measuring miRNA, such as TaqMan(™) MicroRNA Assays (Life Technologies Corp.), LNA(™)—based MicroRNA PCR (Exiqon), or Ncode(™) miRNA qRT-PCT kit (Invitrogen Corp.) may be used.

In the method of the present invention, measurement of the gene expression level(s) may be performed with a sequencer, in addition to hybridization methods described above. In use of a sequencer, any of DNA sequencers of the first generation based on Sanger method, the second generation with shorter read size, and the third generation with longer read size can be used (herein referred to as “next-generation sequencer”, including sequencers of the second generation and the third generation). For example, a commercially available measurement kit specifically designed for measuring miRNA using Miseq, Hiseq, or NexSeq (Illumina, Inc.); Ion Proton, Ion PGM, or Ion S5/S5 XL (Thermo Fisher Scientific Inc.); PacBio RS II or Sequel (Pacific Biosciences of California, Inc.); MinION (Oxford Nanopore Technologies Ltd.) exemplified in use of a Nanopore sequencer; or the like may be used.

Next-generation sequencing is a method of obtaining sequence information using a next-generation sequencer, and characterized by being capable of simultaneously performing a huge number of sequencing reactions compared to Sanger method (e.g., Rick Kamps et al., Int. J. Mol. Sci., 2017, 18(2), p. 308 and Int. Neurourol. J., 2016, 20 (Suppl.2), S76-83). Examples of next-generation sequencing steps for miRNA include, but not limited to, the following steps: at first, adaptor sequences having predetermined nucleotide sequences are attached, and all RNAs are reverse-transcribed into cDNAs before or after attachment of the sequences. After the reverse transcription, cDNAs derived from specific target miRNAs may be enriched or concentrated by PCR or the like or with a probe or the like, for analyzing the target miRNA before sequencing steps. Subsequent sequencing steps varies in detail depending on the type of a next-generation sequencer, but typically, a sequencing reaction is performed by linking to a substrate via an adaptor sequence and further using the adaptor sequence as a priming site. See details of the sequencing reaction, for example, in Rick Kamps et al. (see supra). Finally, the data are outputted. This step provides a collection of sequence information (reads) obtained by the sequencing reaction. For example, next-generation sequencing can identify a target miRNA(s) based on the sequence information, and measure the expression level thereof based on the number of reads having the sequences of the target miRNA(s).

For the calculation of gene expression levels, statistical treatment described in, for example, Statistical analysis of gene expression microarray data (Speed T., Chapman and Hall/CRC), and A beginner's guide Microarray gene expression data analysis (Causton H. C. et al., Blackwell publishing) can be used in the present invention, though the calculation method is not limited thereto. For example, twice, preferably 3 times, more preferably 6 times the standard deviation of the measurement values of the blank spots are added to the average measurement value of the blank spots on the DNA chip, and probe spots having a signal value equal to or larger than the resulting value can be regarded as detection spots. Alternatively, the average measurement value of the blank spots is regarded as a background and can be subtracted from the measurement values of the probe spots to determine gene expression levels. A missing value for a gene expression level can be excluded from the analyte, preferably replaced with the smallest value of the gene expression level in each DNA chip, or more preferably replaced with a value obtained by subtracting 0.1 from a logarithmic value of the smallest value of the gene expression level. In order to eliminate low-signal genes, only a gene having a gene expression level of 2⁶, preferably 2⁸, more preferably 2¹⁰ or larger in 20% or more, preferably 50% or more, more preferably 80% or more of the number of measurement samples can be selected as the analyte. Examples of the normalization of the gene expression level include, but are not limited to, global normalization and quantile normalization (Bolstad, B. M. et al., 2003, Bioinformatics, Vol. 19, p. 185-193).

The present invention also provides a method of detecting an ovarian tumor (or assisting detection thereof) in a subject, comprising measuring target genes or gene expression levels in a sample from the subject using the gene markers (or target nucleic acids) of the present invention, the nucleic acids (or polynucleotides for detection or diagnosis), the kit, or the device (e.g., chip) for detecting the gene marker or a combination thereof; and assigning the expression levels of the target genes in a sample from the subject to a discriminant (discriminant function), which is prepared using gene expression levels of a sample(s) from a subject(s) (for example, a patient(s)) known to have an ovarian tumor and a sample(s) from a subject(s) (also referred to as control animal) having no ovarian tumor, as a training sample(s), and which can distinguishably discriminate the presence or absence of an ovarian tumor, thereby evaluating the presence or absence of the ovarian tumor, for example.

Specifically, the present invention further provides the method comprising a first step of measuring in vitro expression levels of target genes, which are known to determine or evaluate that a subject has an ovarian tumor and/or not, in a plurality of samples, using the gene marker (or target nucleic acid) of the present invention, the nucleic acids (or polynucleotides for detection or diagnosis), the kit, the device (e.g., chip) for detecting the gene marker or a combination thereof; a second step of preparing a discriminant with the measurement values of the expression levels of the target genes obtained in the first step as training samples; a third step of measuring in vitro the expression levels of the target genes in a sample from the subject in the same manner as in the first step; and a fourth step of assigning the measurement values of the expression levels of the target genes obtained in the third step to the discriminant obtained in the second step, and determining or evaluating whether the subject has an ovarian tumor or not on the basis of the results obtained from the discriminant, for example. The above target genes are those that can be detected, for example, by the polynucleotides for detection or diagnosis, the polynucleotides contained in the kit or device, and variants thereof or fragments thereof.

The discriminant herein can be prepared by use of any discriminant analysis method, based on which a discriminant that distinguishably discriminates the presence or absence of an ovarian tumor can be prepared, such as Fisher's discriminant analysis, nonlinear discriminant analysis based on the Mahalanobis' distance, neural network or Support Vector Machine (SVM), though the analysis method is not limited to these specific examples.

When a clustering boundary is a straight line or a hyperplane, the linear discriminant analysis is a method for determining the belonging of a cluster using Formula 1 as a discriminant. In Formula 1, x represents an explanatory variable, w represents a coefficient of the explanatory variable, and wo represents a constant term.

$$f ( x ) = {w}_{0} + {\sum }_{i = 1}^{n} {w}_{i} {x}_{i} \qquad Formula 1$$

Values obtained from the discriminant are referred to as discriminant scores. The measurement values of a newly offered data set can be assigned as explanatory variables to the discriminant to determine clusters by the signs of the discriminant scores.

The Fisher's discriminant analysis, a type of linear discriminant analysis, is a dimensionality reduction method for selecting a dimension suitable for discriminating classes, and constructs a highly discriminating synthetic variable by focusing on the variance of the synthetic variables and minimizing the variance of data having the same label (Venables, W. N. et al., Modern Applied Statistics with S. Fourth edition. Springer, 2002). In the Fisher's discriminant analysis, direction w of projection is determined so as to maximize Formula 2. In this formula, μ represents an average input, ng represents the number of data belonging to class g, and μg represents an average input of the data belonging to class g. The numerator and the denominator are the interclass variance and the intraclass variance, respectively, when each of data is projected in the direction of the vector w. Discriminant coefficient wᵢ is determined by maximizing this ratio (Takafumi Kanamori et al., “Pattern Recognition”, KYORITSU SHUPPAN CO., LTD. (Tokyo, Japan) (2009); Richard O. et al., Pattern Classification, Second Edition., Wiley-Interscience, 2000).

$$J ( w ) = \frac{{\sum }_{g = 1}^{G} {n}_{g} ( {w}^{T} {μ}_{g} - {w}^{T} μ ) {( {w}^{T} {μ}_{g} - {w}^{T} μ )}^{T}}{{\sum }_{g = 1}^{G} \text{?} ( {w}^{T} {x}_{i} - {w}^{T} {μ}_{g} ) ( {w}^{T} {x}_{i} - {w}^{T} {μ}_{g} )} \qquad Formula 2$$

$$subjectto μ = {\sum }_{i = 1}^{n} \frac{{x}_{i}}{n} , {μ}_{g} = \text{?} \frac{{x}_{i}}{{n}_{g}}$$

$$\text{?} \text{indicates text missing or illegible when filed}$$

The Mahalanobis' distance is calculated according to Formula 3 in consideration of data correlation and can be used as nonlinear discriminant analysis for determining a cluster in which a data point belongs to, based on a short Mahalanobis' distance from the data point to that cluster. In Formula 3, μ represents a central vector of each cluster, and S⁻¹ represents an inverse matrix of the variance-covariance matrix of the cluster. The central vector is calculated from explanatory variable x, and an average vector, a median value vector, or the like can be used.

$$D ( x , μ ) = {\{ ( x - μ \text{?} {S}^{- 1} ( x - μ ) \}}^{\frac{1}{2}} \qquad Formula 3$$

$$\text{?} \text{indicates text missing or illegible when filed}$$

SVM is a discriminant analysis method devised by V. Vapnik (The Nature of Statistical Leaning Theory, Springer, 1995). Particular data points of a data set having known classes are defined as explanatory variables, and classes are defined as objective variables. A boundary plane called hyperplane for correctly classifying the data set into the known classes is determined, and a discriminant for data classification is determined using the boundary plane. Then, the measurement values of a newly offered data set can be assigned as explanatory variables to the discriminant to determine classes. In this respect, the result of the discriminant analysis may be classes, may be a probability of being classified into correct classes, or may be the distance from the hyperplane. In SVM, a method of nonlinearly converting a feature vector to a high dimension and performing linear discriminant analysis in the space is known as a method for tackling nonlinear problems. An expression in which an inner product of two factors in a nonlinearly mapped space is expressed only by inputs in their original spaces is called kernel. Examples of the kernel can include a linear kernel, a RBF (Radial Basis Function) kernel, and a Gaussian kernel. While highly dimensional mapping is performed according to the kernel, the optimum discriminant, i.e., a discriminant, can be actually constructed by mere calculation according to the kernel, which avoids calculating features in the mapped space (e.g., Hideki Aso et al., Frontier of Statistical Science 6 “Statistics of pattern recognition and learning—New concepts and approaches”, Iwanami Shoten, Publishers (Tokyo, Japan) (2004); Nello Cristianini et al., Introduction to SVM, Kyoritsu Shuppan Co., Ltd. (Tokyo, Japan) (2008)).

C-support vector classification (C-SVC), a type of SVM, comprises preparing a hyperplane by training a data set with the explanatory variables of two groups and classifying an unknown data set into either of the groups (C. Cortes et al., 1995, Machine Learning, Vol. 20, p. 273-297).

Exemplary calculation of the C-SVC discriminant that can be used in the method of the present invention will be given below. First, all subjects are divided into two groups, i.e., a group of ovarian tumor patients and a group of test subjects having no ovarian tumor. For example, ovarian tissue examination can be used for a reference under which each subject is confirmed to have an ovarian tumor or not.

Next, a data set consisting of comprehensive gene expression levels of serum-derived samples of the two divided groups (hereinafter, this data set is referred to as a training cohort) is prepared, and a C-SVC discriminant is determined by using genes found to differ clearly in their gene expression levels between the two groups as explanatory variables and this grouping as objective variables (e.g., −1 and +1). An optimizing objective function is represented by Formula 4 wherein e represents all input vectors, y represents an objective variable, a represents a Lagrange's undetermined multiplier vector, Q represents a positive definite matrix, and C represents a parameter for adjusting constrained conditions.

$${min}_{a} \frac{1}{2} {a}^{T} Qa - {e}^{T} a \\ subject to {y}^{T} a = 0 , 0 \leq {a}_{i} \leq C , i = 1 , … , l \qquad Formula 4$$

Formula 5 is a finally obtained discriminant, and a group in which the data point belongs to can be determined on the basis of the sign of a value obtained according to the discriminant. In this formula, x represents a support vector, y represents a label indicating the belonging of a group, a represents the corresponding coefficient, b represents a constant term, and K represents a kernel function.

$$f ( x ) = sgn ( \text{?} ) {y}_{i} {a}_{i} K ( {x}_{i} , x ) + b ) \qquad Formula 5$$

$$\text{?} \text{indicates text missing or illegible when filed}$$

For example, a RBF kernel defined by Formula 6 can be used as the kernel function. In this formula, x represents a support vector, and y represents a kernel parameter for adjusting the complexity of the hyperplane.

$$K ( {x}_{i} , {x}_{j} ) = exp ( - r {‖ {x}_{i} - {x}_{j} ‖}^{2} ) , r < 0 \qquad Formula 6$$

In addition, an approach such as neural network, k-nearest neighbor algorithms, decision trees, or logistic regression analysis can be selected as a method for determining or evaluating the presence or absence of ovarian tumor in a sample from a subject.

In an embodiment, the method of the present invention can comprise, for example, the following steps (a), (b) and (c):
(a) a step of measuring an expression level(s) of a target gene(s) in samples already known to be from ovarian tumor patients and to be subjects having no ovarian tumor, using the polynucleotide(s), the kit, or the device (e.g., DNA chip) for detection or diagnosis according to the present invention;
(b) a step of preparing the discriminants of Formulas 1 to 3, 5 and 6 described above from the measurement values of the expression level determined in the step (a), and
(c) a step of measuring an expression level(s) of the target gene(s) in a sample from a subject using the polynucleotide(s), the kit, or the device (e.g., DNA chip) for detection or diagnosis according to the present invention, and assigning the obtained measurement value(s) to the discriminants prepared in the step (b), and determining or evaluating that the subject has an ovarian tumor or not on the basis of the obtained results, or evaluating the expression level derived from an ovarian tumor by comparison with a control from a subject having no ovarian tumor (including, e.g., a healthy subject).

In this context, in the discriminants of Formulas 1 to 3, 5 and 6, x represents an explanatory variable and includes a value obtained by measuring a polynucleotide(s) selected from the polynucleotides serving as a target nucleic acid described in Section 2 above or a fragment thereof. Specifically, the explanatory variable of the present invention for discriminating the presence or absence of an ovarian tumor is a gene expression level(s) selected from, for example, the following expression level (1) or (2).
(1) a gene expression level(s) in the serum of an ovarian tumor patient and a test subject having no ovarian tumor measured by any nucleic acid (e.g., DNA or RNA) comprising 15 or more consecutive nucleotides in the nucleotide sequence represented by any of SEQ ID NOs: 1 to 247, 251 and 268 or a complementary sequence thereof; and
(2) a gene expression level(s) in the serum of an ovarian tumor patient and a test subject having no ovarian tumor measured by any nucleic acid (e.g., DNA or RNA) comprising 15 or more consecutive nucleotides in the nucleotide sequence represented by any of SEQ ID NOs: 248 to 250, 252 to 267 and 269 to 275 or a complementary sequence thereof.

As described above, as the method for determining or evaluating whether a subject has an ovarian tumor or not in a sample from the subject, it is necessary to use a discriminant employing one or more gene expression levels as an explanatory variable(s). In particular, for enhancing the accuracy of the discriminant using a single gene expression level alone, it is necessary to use a gene having a clear difference in expression level between two groups consisting of a group of ovarian tumor patients and a group of subjects having no ovarian tumor, in a discriminant.

Specifically, the gene that is used for an explanatory variable of a discriminant is preferably determined as follows. First, using comprehensive gene expression levels of a group of ovarian tumor patients and comprehensive gene expression levels of a group of test subjects having no ovarian tumor, both of which are in a training cohort, as a data set, the degree of difference in the expression level of each gene between the two groups is obtained by use of, for example, the P value of a parametric analysis such as t-test, the P value of a nonparametric analysis such as the Mann-Whitney's U test or the P value of the Wilcoxon test.

The gene can be regarded as being statistically significant when the critical rate (significance level) as the P value obtained by the test is smaller than, for example, 5%, 1%, or 0.01%.

In order to correct an increased probability of type I error attributed to the repetition of a test, a method known in the art, for example, Bonferroni or Holm method, can be used for the correction (e.g., Yasushi Nagata et al., “Basics of statistical multiple comparison methods”, Scientist Press Co., Ltd. (Tokyo, Japan) (2007)). As an example of the Bonferroni correction, for example, the P value obtained by a test is multiplied by the number of repetitions of the test, i.e., the number of genes used in the analysis, and the obtained value can be compared with a desired significance level to suppress a probability of causing type I error in the whole test.

Instead of the test, the absolute value (fold change) of an expression ratio of a median value of each gene expression level between gene expression levels of a group of patients having ovarian tumor and gene expression levels of a group of test subjects having no ovarian tumor may be calculated to select a gene that is used for an explanatory variable in a discriminant. Alternatively, ROC curves may be prepared using gene expression levels of a group of patients having ovarian tumor and a group of test subjects having no ovarian tumor, and a gene that is used for an explanatory variable in a discriminant can be selected on the basis of an AUROC value.

Next, a discriminant that can be calculated by various methods described above is prepared using any number of genes having large difference in their gene expression levels determined here. Examples of the method for constructing a discriminant that produces the largest discrimination accuracy include a method of constructing a discriminant in every combination of genes that satisfy the significance level being P value, and a method of repetitively evaluating the genes for use in the preparation of a discriminant while increasing the number of genes one by one in a descending order of difference in gene expression level (Furey T S. et al., 2000, Bioinformatics., Vol. 16, p. 906-14). To the discriminant, the gene expression level of another independent patient having an ovarian tumor or a test subject having no ovarian tumor is assigned as an explanatory variable to calculate discrimination results of the group to which the independent patient having an ovarian tumor or the test subject having no ovarian tumor belongs. Specifically, the gene set for diagnosis found and the discriminant constructed using the gene set for diagnosis can be evaluated in an independent sample cohort to find more universal gene set for diagnosis that can detect an ovarian tumor and a more universal method for discriminating an ovarian tumor.

In preparing a discriminant using expression levels of a plurality of genes as an explanatory variable, it is not necessary to select a gene having a clear difference in expression level between the group of ovarian tumor patients and the group of test subjects having no ovarian tumor as described above. Specifically, if expression levels of a plurality of genes are used in combination even though the expression levels of individual genes do not clearly differ, a discriminant having high discriminant performance can be obtained, as the case may be. Because of this, it is preferable to search a discriminant having high discriminant performance without selecting the gene to be employed in the discriminant.

Split-sample method is preferably used for evaluating the performance (generality) of the discriminant. Specifically, a data set is divided into a training cohort and a validation cohort, and gene selection by a statistical test and discriminant preparation are performed using the training cohort. Accuracy, sensitivity, and specificity are calculated using a result of discriminating a validation cohort according to the discriminant, and a true group to which the validation cohort belongs, to evaluate the performance of the discriminant. On the other hand, instead of dividing a data set, the gene selection by a statistical test and discriminant preparation may be performed using all of samples, and accuracy, sensitivity, and specificity can be calculated by the discriminant using a newly prepared sample cohort for evaluation of the performance of the discriminant.

The present invention provides a polynucleotide(s) for diagnosis or detection of a disease useful for diagnosing and treating an ovarian tumor, a method for detecting an ovarian tumor using the polynucleotide(s), and a kit and device for detecting or diagnosing an ovarian tumor, comprising the polynucleotide(s). Particularly, in order to select a gene(s) for diagnosis and prepare a discriminant so as to exhibit accuracy beyond the ovarian tumor diagnosis method using the existing tumor marker CA-125, a gene set for diagnosis and a discriminant for the method of present invention can be constructed, which exhibit accuracy beyond CA-125, for example, by comparing expressed genes in serum from a patient confirmed to be negative using a known tumor marker such as CA-125 but finally found to have an ovarian tumor by detailed examination such as computed tomography using a contrast medium, with genes expressed in serum from a test subject having no ovarian tumor.

For example, the gene set for diagnosis is set to any combination selected from one or two or more of the polynucleotides based on a nucleotide sequence represented by any of SEQ ID NOs: 1 to 247, 251, and 268 as described above; and optionally one or two or more of the polynucleotides based on a nucleotide sequence represented by any of SEQ ID NOs: 248 to 250, 252 to 267, and 269 to 275. Further, a discriminant is constructed using the expression levels of the gene set for diagnosis in samples from an ovarian tumor patient as a result of tissue diagnosis and samples from a test subject having no ovarian tumor as a result of tissue diagnosis. As a result, whether a subject, from which an unknown sample is provided, has an ovarian tumor or not can be determined with 100% accuracy at the maximum by measuring expression levels of the gene set for diagnosis in an unknown sample.

### EXAMPLES

The present invention will be described further specifically with reference to Examples below. However, the scope of the present invention is not intended to be limited by these Examples.

### Reference Example

### <Collection of Samples>

Sera were collected using VENOJECT II vacuum blood collecting tube VP-AS109K60 (Terumo Corp. (Japan)) from 400 healthy subjects, 327 benign bone and soft tissue tumor patients, 41 benign breast disease patients, 406 ovarian cancer patients, 28 ovarian benign tumor patients, and 50 respective patients having breast cancer, biliary cancer, pancreatic cancer, colorectal cancer, esophagus cancer, stomach cancer, liver cancer or lung cancer, after obtainment of informed consent. Note that patients having breast cancer, biliary cancer, pancreatic cancer, colorectal cancer, esophagus cancer, stomach cancer, liver cancer or lung cancer will be collectively referred hereinafter to as “patients having a cancer other than ovarian cancer”.

### <Extraction of Total RNA>

Total RNA was obtained using a reagent for RNA extraction in 3D-Gene(™) RNA extraction reagent from liquid sample kit (Toray Industries, Inc. (Japan)) according to the protocol provided by the manufacturer, from 300 μL of the serum sample obtained from each of 1602 persons in total of 400 healthy subjects, 368 benign bone and soft tissue tumor patients and benign breast disease patients, 434 ovarian tumor patients and 400 patients having a cancer other than ovarian cancer.

### <Measurement of Gene Expression Level>

First, miRNA in the total RNA, which was obtained from the serum samples of a total of 1602 people consisting of 400 healthy subjects, 368 benign bone and soft tissue tumor patients and benign breast disease patients, 434 ovarian tumor patients and 400 patients having a cancer other than ovarian cancer, was fluorescently labeled by use of 3D-Gene(™) miRNA Labeling kit (Toray Industries, Inc.) according to the protocol provided by the manufacturer. The oligo DNA chip used was 3D-Gene(™) Human miRNA Oligo chip (Toray Industries, Inc.) with attached probes having sequences complementary to 2,565 miRNAs among the miRNAs registered in miRBase Release 21. Hybridization under stringent conditions and washing following the hybridization were performed according to the protocol provided by the manufacturer. The DNA chip was scanned using 3D-Gene(™) scanner (Toray Industries, Inc.) to obtain images. Fluorescence intensity was digitized using 3D-Gene(™) Extraction (Toray Industries, Inc.). The digitized fluorescence intensity was converted to a logarithmic value having a base of 2 and used as a gene expression level, from which a blank value was subtracted. A missing value was replaced with a signal value 0.1. As a result, the comprehensive gene expression levels of the miRNAs in the sera were obtained for 400 healthy subjects, 368 benign bone and soft tissue tumor patients and benign breast disease patients, 434 ovarian tumor patients, and 400 patients having a cancer other than ovarian cancer. Subsequently, 70% of each sample group was used as a training cohort and 30% thereof as a validation cohort. Specifically, the training cohort consisted of 280 healthy subjects, 257 benign bone and soft tissue tumor patients and benign breast disease patients, 303 ovarian tumor patients and 280 patients having a cancer other than ovarian cancer; while the validation cohort consisted of 120 healthy subjects, 111 benign bone and soft tissue tumor patients and benign breast disease patients, 131 ovarian tumor patients, and 120 patients having a cancer other than ovarian cancer. Calculation and statistical analysis using the digitized gene expression levels of the miRNAs were carried out using R language 3.3.1 (R Core Team (2016). R: A language and environment for statistical computing. R Foundation for Statistical Computing, Vienna, Austria. URL https://www.R-project.org/.) and MASS package 7.3.45 (Venables, W. N. & Ripley, B. D. (2002) Modern Applied Statistics with S. Fourth Edition. Springer, New York. ISBN 0-387-95457-0).

### Example 1

<Selection of Ovarian Tumor Gene Marker by Comparison with Healthy Subject and Method for Evaluating Ovarian Tumor Discriminant Performance by Single Gene Marker>

In this Example, miRNAs whose expression level significantly differs between ovarian tumor patients and healthy subjects were selected as ovarian tumor gene markers. A discriminant(s) was prepared using one or two gene markers in the training cohort, and then, the discrimination accuracy in the validation cohort was calculated. Based on the calculation, the performance of the gene marker(s) to distinguish ovarian tumor patients from healthy subjects was evaluated.

Specifically, first, the miRNA expression levels of the training cohort and the validation cohort obtained in the preceding Reference Examples were combined and normalized by global normalization.

Next, genes for diagnosis were selected. Here, in order to acquire diagnostic markers with higher reliability, only genes having the expression level of 2⁶ or higher in 50% or more of the samples in either of the ovarian tumor patient group or the healthy subject group were selected. Subsequently, in order to obtain a gene whose expression level significantly differs in statistics between an ovarian tumor patient group and a healthy subject group, a two-sided t-test assuming equal variance was carried out, and then, a gene having a P value obtained after the Bonferroni correction of less than 0.01 was selected. Furthermore, in order to select a gene rarely affected by noise at the time of measurement, a gene(s) having an absolute value of the difference (hold change) in gene expression level, which is obtained by logarithmic conversion between the ovarian tumor patient group and the healthy group, and which is larger than 0.5, was selected as a diagnostic marker(s) for an ovarian tumor and a healthy subject. The results are shown in Table 2.

In this way, hsa-miR-4675, hsa-miR-4783-3p, hsa-miR-1228-5p, hsa-miR-4532, hsa-miR-6802-5p, hsa-miR-6784-5p, hsa-miR-3940-5p, hsa-miR-1307-3p, hsa-miR-8073, hsa-miR-3184-5p, hsa-miR-1233-5p, hsa-miR-6088, hsa-miR-5195-3p, hsa-miR-320b, hsa-miR-4649-5p, hsa-miR-6800-5p, hsa-miR-1343-3p, hsa-miR-4730, hsa-miR-6885-5p, hsa-miR-5100, hsa-miR-1203, hsa-miR-6756-5p, hsa-miR-373-5p, hsa-miR-1268a, hsa-miR-1260b, hsa-miR-4258, hsa-miR-4697-5p, hsa-miR-1469, hsa-miR-4515, hsa-miR-6861-5p, hsa-miR-6821-5p, hsa-miR-575, hsa-miR-6805-5p, hsa-miR-4758-5p, hsa-miR-3663-3p, hsa-miR-4530, hsa-miR-6798-5p, hsa-miR-6781-5p, hsa-miR-885-3p, hsa-miR-1273g-3p, hsa-miR-4787-3p, hsa-miR-4454, hsa-miR-4706, hsa-miR-1249-3p, hsa-miR-887-3p, hsa-miR-6786-5p, hsa-miR-1238-5p, hsa-miR-6749-5p, hsa-miR-6729-5p, hsa-miR-6825-5p, hsa-miR-663b, hsa-miR-6858-5p, hsa-miR-4690-5p, hsa-miR-6765-5p, hsa-miR-4710, hsa-miR-6775-5p, hsa-miR-371a-5p, hsa-miR-6816-5p, hsa-miR-296-3p, hsa-miR-7977, hsa-miR-8069, hsa-miR-6515-3p, hsa-miR-4687-5p, hsa-miR-1343-5p, hsa-miR-7110-5p, hsa-miR-4525, hsa-miR-3158-5p, hsa-miR-6787-5p, hsa-miR-614, hsa-miR-4689, hsa-miR-1185-2-3p, hsa-miR-1268b, hsa-miR-1228-3p, hsa-miR-1185-1-3p, hsa-miR-940, hsa-miR-939-5p, hsa-miR-6757-5p, hsa-miR-1275, hsa-miR-5001-5p, hsa-miR-6826-5p, hsa-miR-6765-3p, hsa-miR-3679-3p, hsa-miR-4718, hsa-miR-4286, hsa-miR-8059, hsa-miR-4447, hsa-miR-4448, hsa-miR-658, hsa-miR-6766-3p, hsa-miR-197-5p, hsa-miR-6887-5p, hsa-miR-6742-5p, hsa-miR-6729-3p, hsa-miR-5090, hsa-miR-7975, hsa-miR-4505, hsa-miR-6889-5p, hsa-miR-4708-3p, hsa-miR-6131, hsa-miR-1225-3p, hsa-miR-6132, hsa-miR-4734, hsa-miR-3194-3p, hsa-miR-638, hsa-miR-2467-3p, hsa-miR-4728-5p, hsa-miR-5572, hsa-miR-6789-5p, hsa-miR-8063, hsa-miR-4429, hsa-miR-6840-3p, hsa-miR-4476, hsa-miR-675-5p, hsa-miR-711, hsa-miR-6875-5p, hsa-miR-3160-5p, hsa-miR-1908-5p, hsa-miR-6726-5p, hsa-miR-1913, hsa-miR-8071, hsa-miR-3648, hsa-miR-4732-5p, hsa-miR-4787-5p, hsa-miR-3917, hsa-miR-619-5p, hsa-miR-1231, hsa-miR-342-5p, hsa-miR-4433a-5p, hsa-miR-6766-5p, hsa-miR-4707-5p, hsa-miR-7114-5p, hsa-miR-6872-3p, hsa-miR-6780b-5p, hsa-miR-7845-5p, hsa-miR-6798-3p, hsa-miR-665, hsa-miR-6848-5p, hsa-miR-5008-5p, hsa-miR-4294, hsa-miR-6511a-5p, hsa-miR-4435, hsa-miR-4747-3p, hsa-miR-6880-3p, hsa-miR-6869-5p, hsa-miR-7150, hsa-miR-1260a, hsa-miR-6877-5p, hsa-miR-6721-5p, hsa-miR-4656, hsa-miR-1229-5p, hsa-miR-4433a-3p, hsa-miR-4274, hsa-miR-4419b, hsa-miR-4674, hsa-miR-6893-5p, hsa-miR-6763-3p, hsa-miR-6762-5p, hsa-miR-6738-5p, hsa-miR-4513, hsa-miR-6746-5p, hsa-miR-6880-5p, hsa-miR-4736, hsa-miR-718, hsa-miR-6717-5p, hsa-miR-7847-3p, hsa-miR-760, hsa-miR-1199-5p, hsa-miR-6813-5p, hsa-miR-6769a-5p, hsa-miR-1193, hsa-miR-7108-3p, hsa-miR-6741-5p, hsa-miR-4298, hsa-miR-6796-3p, hsa-miR-4750-5p, hsa-miR-6785-5p, hsa-miR-1292-3p, hsa-miR-4749-3p, hsa-miR-6800-3p, hsa-miR-4722-5p, hsa-miR-4746-3p, hsa-miR-4450, hsa-miR-6795-5p, hsa-miR-365a-5p, hsa-miR-498, hsa-miR-6797-5p, hsa-miR-1470, hsa-miR-6851-5p, hsa-miR-1247-3p, hsa-miR-5196-5p, hsa-miR-208a-5p, hsa-miR-6842-5p, hsa-miR-150-3p, hsa-miR-4534, hsa-miR-3135b, hsa-miR-3131, hsa-miR-4792, hsa-miR-6510-5p, hsa-miR-504-3p, hsa-miR-3619-3p, hsa-miR-671-5p, hsa-miR-4667-5p, hsa-miR-4430, hsa-miR-320a, hsa-miR-663a, hsa-miR-328-5p, hsa-miR-642b-3p, hsa-miR-128-2-5p, hsa-miR-125a-3p, hsa-miR-191-5p, hsa-miR-92b-5p, hsa-miR-296-5p, hsa-miR-1246, hsa-miR-92a-2-5p, hsa-miR-128-1-5p, hsa-miR-1290, hsa-miR-211-3p, hsa-miR-744-5p, hsa-miR-135a-3p, hsa-miR-451a, hsa-miR-625-3p, hsa-miR-92a-3p, hsa-miR-422a and hsa-miR-642a-3p genes, and the relevant polynucleotides consisting of nucleotide sequences of SEQ ID NOs: 1 to 203 and 248 to 268 were found.

Of them, the genes newly found as the marker for examining the presence or absence of an ovarian tumor are polynucleotides consisting of the nucleotide sequences represented by SEQ ID NOs: 1 to 203.

A discriminant for determining the presence or absence of an ovarian tumor was further prepared by the Fisher's discriminant analysis with the expression levels of these genes in the training cohort as indicators. Specifically, the gene expression level of a polynucleotide consisting of the nucleotide sequence represented by any of SEQ ID NOs: 1 to 203 and 248 to 268 found above was input to Formula 2 to prepare a discriminant. Calculated accuracy, sensitivity, and specificity in the training cohort are shown in Table 3. In this respect, discriminant coefficients and constant terms are shown in Table 4.

Next, accuracy, sensitivity, and specificity in the validation cohort were calculated using the discriminant prepared above and the discriminant performance of the selected polynucleotides was validated using independent samples (Table 3). For example, the rate of detecting an ovarian tumor was calculated using the threshold value (7.75) of the gene expression level of the nucleotide sequence represented by SEQ ID NO: 1, for use in discriminating both groups set in the training cohort. As a result, 127 true positives, 117 true negatives, 3 false positives and 4 false negatives were obtained in the validation cohort. From these values, 97.2% accuracy, 96.9% sensitivity, and 97.5% specificity were obtained as the detection performance. In this way, the detection performance values of all polynucleotides represented by SEQ ID NOs: 1 to 203 and 248 to 268 were calculated and described in Table 3.

For example, 143 polynucleotides consisting of the nucleotide sequences represented by SEQ ID NOs: 1 to 78, 80 to 90, 92 to 94, 97, 99, 100, 104, 107, 109 to 111, 113, 115, 119, 121 to 125, 128, 129, 135 to 138, 142 to 146, 148 to 150, 152, 153, 156, 158, 159, 248 to 262 and 265 exhibited sensitivities of 97.2%, 94.4%, 98.0%, 94.4%, 94.8%, 97.6%, 94.4%, 93.6%, 92.8%, 95.6%, 92.8%, 93.2%, 90.8%, 94.4%, 90.0%, 97.6%, 91.2%, 92.0%, 88.0%, 88.8%, 90.8%, 89.2%, 90.8%, 86.9%, 86.9%, 89.2%, 88.4%, 89.2%, 88.0%, 88.4%, 90.0%, 86.1%, 89.2%, 87.6%, 90.8%, 86.1%, 85.3%, 91.2%, 89.6%, 86.5%, 89.2%, 84.9%, 86.5%, 88.4%, 88.8%, 86.9%, 90.4%, 85.7%, 85.3%, 84.5%, 83.7%, 83.3%, 88.4%, 86.5%, 80.5%, 84.9%, 87.3%, 83.7%, 87.6%, 79.7%, 85.3%, 84.1%, 90.8%, 82.9%, 84.9%, 79.7%, 89.2%, 83.3%, 78.5%, 83.7%, 82.1%, 81.3%, 88.0%, 81.3%, 79.7%, 84.5%, 82.9%, 83.7%, 80.1%, 77.7%, 84.9%, 79.7%, 81.3%, 77.7%, 83.3%, 78.5%, 86.5%, 78.9%, 79.7%, 82.9%, 86.5%, 87.6%, 80.1%, 80.5%, 78.5%, 79.3%, 78.9%, 80.5%, 78.1%, 80.5%, 86.1%, 78.9%, 82.5%, 80.9%, 82.1%, 78.5%, 80.9%, 78.1%, 82.9%, 80.1%, 79.7%, 78.9%, 80.5%, 78.5%, 80.1%, 81.3%, 77.7%, 78.9%, 80.1%, 79.3%, 79.3%, 77.7%, 80.1%, 78.9%, 80.1%, 77.7%, 77.7%, 97.6%, 93.2%, 94.8%, 93.2%, 88.4%, 90.0%, 88.8%, 90.4%, 92.4%, 83.7%, 88.0%, 93.2%, 85.7%, 84.9%, 79.3%, and 79.3%, respectively in the validation cohort (Table 3). Herein, a general sensitivity of the existing marker CA-125 is reported to be 77.4% (Non Patent Literature 4). Accordingly, it was demonstrated that the polynucleotides consisting of these sequences singly discriminate an ovarian tumor with sensitivity beyond CA-125.

Also, a discriminant was constructed using each of all polynucleotides represented by SEQ ID NOs: 1 to 203 and 248 to 268 in combination with another polynucleotide appropriately selected; in other words, discriminants each were constructed using two polynucleotides. The discrimination accuracy of an ovarian tumor in the validation cohort by the discriminants thus constructed increased, compared with the discriminants using individual genes alone. Specifically, discriminants prepared using combinations of SEQ ID NOs: 1 and 3, 2 and 3, 1 and 3, 3 and 4, 3 and 5, 3 and 6, 3 and 7, 6 and 8, 3 and 9, 248 and 10, 11 and 18, 3 and 12, 3 and 13, 14 and 16, 6 and 15, 248 and 16, 6 and 17, 248 and 18, 3 and 19, 3 and 20, 3 and 21, 3 and 22, 3 and 23, 3 and 24, 1 and 25, 3 and 26, 3 and 27, 1 and 28, 3 and 29, 16 and 30, 3 and 31, 3 and 32, 3 and 33, 6 and 34, 3 and 35, 3 and 36, 3 and 37, 11 and 38, 3 and 39, 3 and 40, 3 and 41, 3 and 42, 3 and 43, 3 and 44, 3 and 45, 3 and 46, 248 and 47, 6 and 48, 249 and 49, 3 and 50, 3 and 51, 3 and 52, 16 and 53, 3 and 54, 7 and 55, 248 and 56, 3 and 57, 249 and 58, 3 and 59, 1 and 60, 249 and 61, 3 and 62, 3 and 63, 248 and 64, 3 and 65, 3 and 66, 3 and 67, 16 and 68, 3 and 69, 3 and 70, 248 and 71, 3 and 72, 3 and 73, 3 and 74, 1 and 75, 3 and 76, 3 and 77, 3 and 78, 1 and 79, 18 and 80, 3 and 81, 3 and 82, 3 and 83, 3 and 84, 10 and 85, 16 and 86, 16 and 87, 3 and 88, 3 and 89, 3 and 90, 248 and 91, 249 and 92, 3 and 93, 3 and 94, 3 and 95, 1 and 96, 3 and 97, 16 and 98, 6 and 99, 10 and 100, 1 and 101, 6 and 102, 3 and 103, 249 and 104, 16 and 105, 3 and 106, 3 and 107, 7 and 108, 3 and 109, 10 and 110, 248 and 111, 3 and 112, 248 and 113, 249 and 114, 248 and 115, 3 and 116, 249 and 117, 3 and 118, 3 and 119, 3 and 120, 3 and 121, 3 and 122, 3 and 123, 3 and 124, 3 and 125, 248 and 126, 10 and 127, 248 and 128, 16 and 129, 3 and 130, 3 and 131, 3 and 132, 1 and 133, 3 and 134, 3 and 135, 10 and 136, 3 and 137, 3 and 138, 249 and 139, 10 and 140, 3 and 141, 3 and 142, 3 and 143, 19 and 144, 3 and 145, 3 and 146, 3 and 147, 248 and 148, 3 and 149, 3 and 150, 3 and 151, 3 and 152, 3 and 153, 3 and 154, 3 and 155, 10 and 156, 3 and 157, 248 and 158, 10 and 159, 10 and 160, 6 and 161, 16 and 162, 3 and 163, 248 and 164, 248 and 165, 3 and 166, 3 and 167, 248 and 168, 3 and 169, 3 and 170, 3 and 171, 3 and 172, 3 and 173, 3 and 174, 3 and 175, 3 and 176, 3 and 177, 6 and 178, 3 and 179, 3 and 180, 3 and 181, 3 and 182, 3 and 183, 3 and 184, 3 and 185, 18 and 186, 3 and 187, 1 and 188, 10 and 189, 3 and 190, 3 and 191, 6 and 192, 3 and 193, 3 and 194, 3 and 195, 3 and 196, 3 and 197, 10 and 198, 3 and 199, 3 and 200, 3 and 201, 250 and 202, 248 and 203, 248 and 10, 1 and 249, 3 and 250, 251 and 18, 6 and 252, 248 and 253, 6 and 254, 3 and 255, 3 and 256, 3 and 257, 3 and 258, 3 and 259, 16 and 260, 10 and 261, 3 and 262, 3 and 263, 3 and 264, 3 and 265, 3 and 266, 248 and 267, and 3 and 268 had discrimination accuracies, in the validation cohort, of 100%, 98.8%, 100%, 99.6%, 100%, 99.6%, 99.6%, 99.2%, 99.2%, 100%, 100%, 99.2%, 99.6%, 100%, 99.2%, 100%, 99.2%, 100%, 99.2%, 99.2%, 98.8%, 99.2%, 99.6%, 99.2%, 99.2%, 98.4%, 99.6%, 99.2%, 99.6%, 99.2%, 99.6%, 99.2%, 98.8%, 99.2%, 98.8%, 99.2%, 99.6%, 99.2%, 99.2%, 99.2%, 98.8%, 99.2%, 99.2%, 98.8%, 99.2%, 98.8%, 99.2%, 98.8%, 99.2%, 98.8%, 98.8%, 98.8%, 99.6%, 99.6%, 97.2%, 99.6%, 99.2%, 99.6%, 98.8%, 98.8%, 98.8%, 99.2%, 99.2%, 98%, 99.2%, 99.2%, 98.8%, 99.6%, 98.4%, 99.2%, 98.8%, 99.6%, 98.8%, 99.2%, 98.8%, 98.4%, 99.2%, 99.6%, 99.6%, 99.6%, 98.4%, 98.8%, 99.2%, 98.4%, 98.8%, 99.6%, 99.2%, 98.8%, 98.8%, 98.8%, 98%, 99.2%, 98.8%, 99.2%, 98.4%, 98.4%, 99.2%, 99.2%, 98.8%, 99.2%, 99.2%, 98.8%, 98.4%, 99.6%, 100%, 99.2%, 98.8%, 98.4%, 98.8%, 98.8%, 98.4%, 99.2%, 98.8%, 99.6%, 99.6%, 98.8%, 99.6%, 98.4%, 99.2%, 98.8%, 99.2%, 98.4%, 98.8%, 98.8%, 98.4%, 98.8%, 99.2%, 98%, 98.8%, 98.4%, 98.8%, 98.4%, 99.6%, 98.8%, 98.4%, 98.8%, 99.2%, 98.4%, 98.8%, 99.2%, 98.8%, 99.2%, 98.4%, 99.2%, 99.2%, 98.4%, 98.4%, 98%, 98.8%, 99.2%, 98.8%, 98.8%, 98.4%, 99.2%, 98.8%, 98.8%, 98.4%, 98.4%, 98.4%, 99.6%, 98.4%, 99.6%, 98.4%, 98.8%, 98%, 98.8%, 98.8%, 98%, 98.4%, 98.4%, 98.4%, 98.4%, 98.4%, 98.8%, 98.4%, 98.8%, 98.4%, 98.4%, 98.4%, 98.4%, 98.4%, 98.8%, 98.4%, 98.4%, 98.4%, 95.6%, 98.8%, 98%, 98.4%, 98.4%, 98.4%, 98%, 98.8%, 98.4%, 98.4%, 98.4%, 98.4%, 98.8%, 98.8%, 98.4%, 98.8%, 97.6%, 98.4%, 100%, 99.6%, 100%, 100%, 99.2%, 99.2%, 99.6%, 98.8%, 99.6%, 99.2%, 98.8%, 99.2%, 100%, 98.8%, 99.6%, 98.4%, 98.4%, 98.8%, 98.4%, 98.8%, and 98.8%, respectively (Table 5). These all exceed discriminant performance of the existing marker CA-125. Note that, in Table 5, in the column of “SEQ ID No”, the combinations of a plurality of polynucleotides used are described by SEQ ID Nos. (the same applies to the tables later described).

From the above, it was demonstrated that all polynucleotides consisting of the nucleotide sequences represented by SEQ ID NOs: 1 to 203 and 248 to 268 are genes capable of discriminating an ovarian tumor patient and a healthy subject with high accuracy if these are used singly or in combinations of two or more.

TABLE 2

| | | | Fold change |
|---|---|---|---|
| | | P value | of healthy |
| SEQ | | after | subject to |
| ID | | Bonferroni | ovarian tumor |
| NO. | Name of gene | correction | patient |
| 1 | hsa-miR-4675 | 4.96E−307 | −2.00 |
| 2 | hsa-miR-4783-3p | 2.73E−296 | −3.02 |
| 3 | hsa-miR-1228-5p | 2.40E−291 | 0.83 |
| 4 | hsa-miR-4532 | 1.59E−270 | −2.71 |
| 5 | hsa-miR-6802-5p | 1.40E−264 | −1.13 |
| 6 | hsa-miR-6784-5p | 8.08E−257 | 1.35 |
| 7 | hsa-miR-3940-5p | 5.59E−227 | 0.98 |
| 8 | hsa-miR-1307-3p | 1.31E−224 | −2.79 |
| 9 | hsa-miR-8073 | 6.97E−223 | −2.32 |
| 10 | hsa-miR-3184-5p | 2.06E−214 | 2.53 |
| 11 | hsa-miR-1233-5p | 4.37E−211 | −2.33 |
| 12 | hsa-miR-6088 | 5.55E−203 | −1.27 |
| 13 | hsa-miR-5195-3p | 1.21E−200 | −1.61 |
| 14 | hsa-miR-320b | 1.36E−198 | −3.20 |
| 15 | hsa-miR-4649-5p | 2.36E−193 | −1.81 |
| 16 | hsa-miR-6800-5p | 1.67E−188 | 1.31 |
| 17 | hsa-miR-1343-3p | 1.81E−180 | −1.65 |
| 18 | hsa-miR-4730 | 2.00E−178 | 2.35 |
| 19 | hsa-miR-6885-5p | 8.17E−177 | −1.59 |
| 20 | hsa-miR-5100 | 2.57E−176 | −2.85 |
| 21 | hsa-miR-1203 | 1.01E−173 | 2.18 |
| 22 | hsa-miR-6756-5p | 1.89E−168 | −0.88 |
| 23 | hsa-miR-373-5p | 6.84E−164 | −1.60 |
| 24 | hsa-miR-1268a | 9.51E−161 | 0.69 |
| 25 | hsa-miR-1260b | 3.63E−160 | −1.66 |
| 26 | hsa-miR-4258 | 4.68E−160 | −1.77 |
| 27 | hsa-miR-4697-5p | 3.86E−158 | −0.99 |
| 28 | hsa-miR-1469 | 6.86E−158 | −1.19 |
| 29 | hsa-miR-4515 | 1.11E−153 | −3.34 |
| 30 | hsa-miR-6861-5p | 1.34E−150 | −1.05 |
| 31 | hsa-miR-6821-5p | 1.36E−150 | −0.54 |
| 32 | hsa-miR-575 | 2.21E−150 | 2.12 |
| 33 | hsa-miR-6805-5p | 7.36E−150 | 0.84 |
| 34 | hsa-miR-4758-5p | 7.32E−148 | −0.89 |
| 35 | hsa-miR-3663-3p | 1.12E−147 | −1.08 |
| 36 | hsa-miR-4530 | 1.32E−144 | 1.04 |
| 37 | hsa-miR-6798-5p | 1.72E−143 | 0.94 |
| 38 | hsa-miR-6781-5p | 2.15E−142 | 0.83 |
| 39 | hsa-miR-885-3p | 1.27E−139 | −2.10 |
| 40 | hsa-miR-1273g-3p | 5.04E−139 | −1.80 |
| 41 | hsa-miR-4787-3p | 1.23E−136 | −1.73 |
| 42 | hsa-miR-4454 | 1.31E−135 | −2.01 |
| 43 | hsa-miR-4706 | 1.55E−131 | −1.01 |
| 44 | hsa-miR-1249-3p | 1.50E−130 | −0.98 |
| 45 | hsa-miR-887-3p | 1.57E−129 | 1.29 |
| 46 | hsa-miR-6786-5p | 6.98E−129 | 0.54 |
| 47 | hsa-miR-1238-5p | 4.70E−128 | −1.93 |
| 48 | hsa-miR-6749-5p | 2.87E−127 | −0.72 |
| 49 | hsa-miR-6729-5p | 3.83E−124 | 0.82 |
| 50 | hsa-miR-6825-5p | 7.08E−124 | 1.72 |
| 51 | hsa-miR-663b | 1.78E−123 | −1.27 |
| 52 | hsa-miR-6858-5p | 1.20E−122 | −0.82 |
| 53 | hsa-miR-4690-5p | 2.08E−119 | −1.23 |
| 54 | hsa-miR-6765-5p | 1.17E−116 | 0.57 |
| 55 | hsa-miR-4710 | 2.39E−115 | −2.97 |
| 56 | hsa-miR-6775-5p | 5.02E−115 | −0.76 |
| 57 | hsa-miR-371a-5p | 1.87E−114 | −1.02 |
| 58 | hsa-miR-6816-5p | 4.12E−113 | 0.56 |
| 59 | hsa-miR-296-3p | 5.77E−113 | −1.79 |
| 60 | hsa-miR-7977 | 2.70E−111 | −1.57 |
| 61 | hsa-miR-8069 | 3.35E−109 | 0.74 |
| 62 | hsa-miR-6515-3p | 7.14E−108 | −0.90 |
| 63 | hsa-miR-4687-5p | 1.19E−107 | −1.21 |
| 64 | hsa-miR-1343-5p | 1.57E−107 | 0.51 |
| 65 | hsa-miR-7110-5p | 3.09E−106 | 1.30 |
| 66 | hsa-miR-4525 | 8.77E−106 | −2.49 |
| 67 | hsa-miR-3158-5p | 3.78E−105 | −1.90 |
| 68 | hsa-miR-6787-5p | 4.50E−105 | −0.86 |
| 69 | hsa-miR-614 | 4.58E−105 | −1.96 |
| 70 | hsa-miR-4689 | 1.93E−103 | −0.67 |
| 71 | hsa-miR-1185-2-3p | 4.33E−101 | −1.56 |
| 72 | hsa-miR-1268b | 2.66E−95 | 0.53 |
| 73 | hsa-miR-1228-3p | 4.24E−95 | −0.69 |
| 74 | hsa-miR-1185-1-3p | 9.39E−94 | −1.46 |
| 75 | hsa-miR-940 | 5.02E−93 | −1.05 |
| 76 | hsa-miR-939-5p | 2.03E−92 | 1.32 |
| 77 | hsa-miR-6757-5p | 3.72E−92 | −1.47 |
| 78 | hsa-miR-1275 | 1.36E−91 | −0.86 |
| 79 | hsa-miR-5001-5p | 8.91E−91 | −0.77 |
| 80 | hsa-miR-6826-5p | 1.03E−90 | −1.30 |
| 81 | hsa-miR-6765-3p | 1.75E−90 | −1.40 |
| 82 | hsa-miR-3679-3p | 9.05E−90 | −0.90 |
| 83 | hsa-miR-4718 | 5.79E−89 | −3.06 |
| 84 | hsa-miR-4286 | 4.60E−88 | −1.51 |
| 85 | hsa-miR-8059 | 3.10E−87 | −1.52 |
| 86 | hsa-miR-4447 | 6.80E−86 | −1.13 |
| 87 | hsa-miR-4448 | 4.46E−83 | −2.39 |
| 88 | hsa-miR-658 | 4.20E−82 | −1.18 |
| 89 | hsa-miR-6766-3p | 3.46E−81 | −0.93 |
| 90 | hsa-miR-197-5p | 5.57E−81 | −1.18 |
| 91 | hsa-miR-6887-5p | 4.12E−80 | −1.24 |
| 92 | hsa-miR-6742-5p | 9.48E−80 | −1.33 |
| 93 | hsa-miR-6729-3p | 1.41E−79 | −0.94 |
| 94 | hsa-miR-5090 | 3.17E−79 | 1.03 |
| 95 | hsa-miR-7975 | 7.82E−79 | −1.52 |
| 96 | hsa-miR-4505 | 4.34E−77 | −0.74 |
| 97 | hsa-miR-6889-5p | 4.87E−76 | 0.88 |
| 98 | hsa-miR-4708-3p | 6.45E−75 | −2.41 |
| 99 | hsa-miR-6131 | 3.49E−72 | −3.15 |
| 100 | hsa-miR-1225-3p | 1.65E−71 | −0.75 |
| 101 | hsa-miR-6132 | 4.26E−71 | −0.83 |
| 102 | hsa-miR-4734 | 7.61E−71 | −0.87 |
| 103 | hsa-miR-3194-3p | 2.18E−70 | −2.78 |
| 104 | hsa-miR-638 | 4.09E−70 | 0.63 |
| 105 | hsa-miR-2467-3p | 5.90E−70 | −2.32 |
| 106 | hsa-miR-4728-5p | 1.30E−69 | −0.83 |
| 107 | hsa-miR-5572 | 1.81E−69 | 1.05 |
| 108 | hsa-miR-6789-5p | 5.37E−69 | −0.56 |
| 109 | hsa-miR-8063 | 9.86E−69 | −1.28 |
| 110 | hsa-miR-4429 | 1.64E−68 | −1.41 |
| 111 | hsa-miR-6840-3p | 4.67E−68 | −0.77 |
| 112 | hsa-miR-4476 | 1.61E−67 | 1.44 |
| 113 | hsa-miR-675-5p | 9.52E−67 | −0.84 |
| 114 | hsa-miR-711 | 2.88E−64 | 0.66 |
| 115 | hsa-miR-6875-5p | 6.25E−64 | 0.99 |
| 116 | hsa-miR-3160-5p | 3.59E−62 | −2.33 |
| 117 | hsa-miR-1908-5p | 4.73E−62 | 0.56 |
| 118 | hsa-miR-6726-5p | 1.03E−61 | −0.77 |
| 119 | hsa-miR-1913 | 3.07E−60 | −0.66 |
| 120 | hsa-miR-8071 | 5.14E−60 | 1.20 |
| 121 | hsa-miR-3648 | 1.35E−59 | −0.87 |
| 122 | hsa-miR-4732-5p | 2.69E−59 | −1.98 |
| 123 | hsa-miR-4787-5p | 3.13E−59 | 0.53 |
| 124 | hsa-miR-3917 | 4.64E−59 | −0.94 |
| 125 | hsa-miR-619-5p | 7.20E−58 | −1.58 |
| 126 | hsa-miR-1231 | 2.21E−57 | 0.98 |
| 127 | hsa-miR-342-5p | 5.05E−57 | −1.66 |
| 128 | hsa-miR-4433a-5p | 5.32E−56 | −0.84 |
| 129 | hsa-miR-6766-5p | 1.42E−53 | −1.03 |
| 130 | hsa-miR-4707-5p | 4.15E−53 | −0.52 |
| 131 | hsa-miR-7114-5p | 1.40E−52 | 0.85 |
| 132 | hsa-miR-6872-3p | 2.48E−52 | −1.01 |
| 133 | hsa-miR-6780b-5p | 4.10E−52 | −0.62 |
| 134 | hsa-miR-7845-5p | 2.32E−51 | 0.91 |
| 135 | hsa-miR-6798-3p | 6.04E−51 | −0.96 |
| 136 | hsa-miR-665 | 9.73E−51 | −0.80 |
| 137 | hsa-miR-6848-5p | 1.70E−50 | 0.73 |
| 138 | hsa-miR-5008-5p | 2.00E−50 | −1.34 |
| 139 | hsa-miR-4294 | 1.56E−49 | 0.58 |
| 140 | hsa-miR-6511a-5p | 3.13E−49 | −0.91 |
| 141 | hsa-miR-4435 | 4.39E−49 | 1.64 |
| 142 | hsa-miR-4747-3p | 7.86E−49 | −1.02 |
| 143 | hsa-miR-6880-3p | 3.19E−48 | −0.76 |
| 144 | hsa-miR-6869-5p | 6.95E−48 | 0.66 |
| 145 | hsa-miR-7150 | 7.08E−48 | −0.57 |
| 146 | hsa-miR-1260a | 1.22E−47 | −0.90 |
| 147 | hsa-miR-6877-5p | 7.53E−47 | −0.67 |
| 148 | hsa-miR-6721-5p | 9.70E−47 | 0.79 |
| 149 | hsa-miR-4656 | 1.13E−46 | −0.55 |
| 150 | hsa-miR-1229-5p | 1.72E−46 | −0.57 |
| 151 | hsa-miR-4433a-3p | 2.51E−46 | −0.52 |
| 152 | hsa-miR-4274 | 6.43E−46 | −0.71 |
| 153 | hsa-miR-4419b | 3.59E−44 | −1.11 |
| 154 | hsa-miR-4674 | 1.71E−40 | 0.71 |
| 155 | hsa-miR-6893-5p | 1.48E−38 | 0.69 |
| 156 | hsa-miR-6763-3p | 2.51E−38 | −0.67 |
| 157 | hsa-miR-6762-5p | 1.13E−37 | −0.64 |
| 158 | hsa-miR-6738-5p | 2.42E−37 | −0.73 |
| 159 | hsa-miR-4513 | 2.19E−35 | −0.66 |
| 160 | hsa-miR-6746-5p | 2.84E−35 | −0.76 |
| 161 | hsa-miR-6880-5p | 4.90E−35 | 0.95 |
| 162 | hsa-miR-4736 | 1.07E−34 | −0.89 |
| 163 | hsa-miR-718 | 3.73E−34 | −0.57 |
| 164 | hsa-miR-6717-5p | 2.68E−33 | −1.29 |
| 165 | hsa-miR-7847-3p | 8.51E−33 | −0.78 |
| 166 | hsa-miR-760 | 4.19E−31 | 0.66 |
| 167 | hsa-miR-1199-5p | 5.43E−31 | −0.73 |
| 168 | hsa-miR-6813-5p | 1.37E−30 | 0.76 |
| 169 | hsa-miR-6769a-5p | 2.48E−29 | −0.75 |
| 170 | hsa-miR-1193 | 2.51E−29 | −0.62 |
| 171 | hsa-miR-7108-3p | 6.05E−28 | −0.59 |
| 172 | hsa-miR-6741-5p | 1.26E−27 | −0.64 |
| 173 | hsa-miR-4298 | 1.63E−27 | −0.65 |
| 174 | hsa-miR-6796-3p | 4.73E−27 | −0.59 |
| 175 | hsa-miR-4750-5p | 6.28E−27 | −0.63 |
| 176 | hsa-miR-6785-5p | 4.16E−26 | 0.64 |
| 177 | hsa-miR-1292-3p | 7.45E−26 | −0.60 |
| 178 | hsa-miR-4749-3p | 1.01E−25 | −0.51 |
| 179 | hsa-miR-6800-3p | 9.04E−25 | −0.51 |
| 180 | hsa-miR-4722-5p | 9.15E−25 | −0.55 |
| 181 | hsa-miR-4746-3p | 2.32E−24 | 0.52 |
| 182 | hsa-miR-4450 | 4.34E−24 | 0.89 |
| 183 | hsa-miR-6795-5p | 4.71E−24 | −0.65 |
| 184 | hsa-miR-365a-5p | 1.05E−23 | −0.52 |
| 185 | hsa-miR-498 | 5.72E−22 | −0.51 |
| 186 | hsa-miR-6797-5p | 1.89E−21 | −0.69 |
| 187 | hsa-miR-1470 | 2.34E−21 | −0.56 |
| 188 | hsa-miR-6851-5p | 7.59E−21 | 0.55 |
| 189 | hsa-miR-1247-3p | 1.84E−20 | −0.54 |
| 190 | hsa-miR-5196-5p | 1.34E−18 | −0.55 |
| 191 | hsa-miR-208a-5p | 2.20E−18 | −0.79 |
| 192 | hsa-miR-6842-5p | 2.95E−18 | 0.54 |
| 193 | hsa-miR-150-3p | 6.45E−18 | 0.60 |
| 194 | hsa-miR-4534 | 7.92E−18 | −0.66 |
| 195 | hsa-miR-3135b | 3.33E−17 | −0.51 |
| 196 | hsa-miR-3131 | 1.08E−15 | −0.58 |
| 197 | hsa-miR-4792 | 1.21E−15 | 0.60 |
| 198 | hsa-miR-6510-5p | 3.26E−15 | −0.53 |
| 199 | hsa-miR-504-3p | 4.36E−15 | −0.56 |
| 200 | hsa-miR-3619-3p | 4.56E−14 | −0.53 |
| 201 | hsa-miR-671-5p | 8.88E−13 | −0.50 |
| 202 | hsa-miR-4667-5p | 8.26E−12 | 0.50 |
| 203 | hsa-miR-4430 | 1.84E−09 | −0.52 |
| 248 | hsa-miR-320a | 6.45E−226 | −2.63 |
| 249 | hsa-miR-663a | 8.51E−226 | −1.68 |
| 250 | hsa-miR-328-5p | 7.43E−192 | −0.58 |
| 251 | hsa-miR-642b-3p | 2.09E−186 | −1.92 |
| 252 | hsa-miR-128-2-5p | 1.24E−177 | −1.68 |
| 253 | hsa-miR-125a-3p | 7.31E−175 | 2.85 |
| 254 | hsa-miR-191-5p | 1.36E−174 | -4.42 |
| 255 | hsa-miR-92b-5p | 2.93E−156 | −1.19 |
| 256 | hsa-miR-296-5p | 1.66E−155 | −1.02 |
| 257 | hsa-miR-1246 | 5.89E−152 | −5.34 |
| 258 | hsa-miR-92a-2-5p | 1.32E−143 | 1.64 |
| 259 | hsa-miR-128-1-5p | 8.05E−142 | 1.57 |
| 260 | hsa-miR-1290 | 6.57E−134 | −4.66 |
| 261 | hsa-miR-211-3p | 4.84E−106 | −1.24 |
| 262 | hsa-miR-744-5p | 2.06E−94 | −1.08 |
| 263 | hsa-miR-135a-3p | 4.88E−79 | 1.43 |
| 264 | hsa-miR-451a | 1.22E−75 | −3.37 |
| 265 | hsa-miR-625-3p | 1.43E−61 | −1.10 |
| 266 | hsa-miR-92a-3p | 5.16E−53 | −1.61 |
| 267 | hsa-miR-422a | 2.70E−40 | −1.08 |
| 268 | hsa-miR-642a-3p | 4.24E−18 | −0.58 |

TABLE 3

| | Training cohort | | | Validation cohort | | |
|---|---|---|---|---|---|---|
| | Accuracy | Sensitivity | Specificity | Accuracy | Sensitivity | |
| SEQ ID NO. | (%) | (%) | (%) | (%) | (%) | Specificity (%) |
| 1 | 96.9 | 98.3 | 95.4 | 97.2 | 96.9 | 97.5 |
| 2 | 96.1 | 92.4 | 100.0 | 94.4 | 89.3 | 100.0 |
| 3 | 97.6 | 96.7 | 98.6 | 98.0 | 96.9 | 99.2 |
| 4 | 95.2 | 91.4 | 99.3 | 94.4 | 89.3 | 100.0 |
| 5 | 96.6 | 97.0 | 96.1 | 94.8 | 92.4 | 97.5 |
| 6 | 96.2 | 98.0 | 94.3 | 97.6 | 98.5 | 96.7 |
| 7 | 93.3 | 87.8 | 99.3 | 94.4 | 90.8 | 98.3 |
| 8 | 95.9 | 92.1 | 100.0 | 93.6 | 87.8 | 100.0 |
| 9 | 94.5 | 95.4 | 93.6 | 92.8 | 91.6 | 94.2 |
| 10 | 96.7 | 93.7 | 100.0 | 95.6 | 91.6 | 100.0 |
| 11 | 93.8 | 88.8 | 99.3 | 92.8 | 87.0 | 99.2 |
| 12 | 90.9 | 89.1 | 92.9 | 93.2 | 91.6 | 95.0 |
| 13 | 93.5 | 93.4 | 93.6 | 90.8 | 87.8 | 94.2 |
| 14 | 93.1 | 92.1 | 94.3 | 94.4 | 96.9 | 91.7 |
| 15 | 91.6 | 91.4 | 91.8 | 90.0 | 87.8 | 92.5 |
| 16 | 97.1 | 97.4 | 96.8 | 97.6 | 96.2 | 99.2 |
| 17 | 91.1 | 89.4 | 92.9 | 91.2 | 89.3 | 93.3 |
| 18 | 91.4 | 88.8 | 94.3 | 92.0 | 93.9 | 90.0 |
| 19 | 90.6 | 87.8 | 93.6 | 88.0 | 82.4 | 94.2 |
| 20 | 90.9 | 92.7 | 88.9 | 88.8 | 92.4 | 85.0 |
| 21 | 90.7 | 83.8 | 98.2 | 90.8 | 84.0 | 98.3 |
| 22 | 89.2 | 91.1 | 87.1 | 89.2 | 86.3 | 92.5 |
| 23 | 91.4 | 88.1 | 95.0 | 90.8 | 88.5 | 93.3 |
| 24 | 88.3 | 88.8 | 87.9 | 86.9 | 80.9 | 93.3 |
| 25 | 88.5 | 87.5 | 89.6 | 86.9 | 87.0 | 86.7 |
| 26 | 89.5 | 87.5 | 91.8 | 89.2 | 84.0 | 95.0 |
| 27 | 91.3 | 89.4 | 93.2 | 88.4 | 82.4 | 95.0 |
| 28 | 89.9 | 83.5 | 96.8 | 89.2 | 80.2 | 99.2 |
| 29 | 90.7 | 95.0 | 86.1 | 88.0 | 92.4 | 83.3 |
| 30 | 90.1 | 88.8 | 91.4 | 88.4 | 82.4 | 95.0 |
| 31 | 88.2 | 90.1 | 86.1 | 90.0 | 93.9 | 85.8 |
| 32 | 90.9 | 85.5 | 96.8 | 86.1 | 77.1 | 95.8 |
| 33 | 90.2 | 85.5 | 95.4 | 89.2 | 84.0 | 95.0 |
| 34 | 88.0 | 82.8 | 93.6 | 87.6 | 78.6 | 97.5 |
| 35 | 90.7 | 93.7 | 87.5 | 90.8 | 90.1 | 91.7 |
| 36 | 88.9 | 91.1 | 86.4 | 86.1 | 85.5 | 86.7 |
| 37 | 85.4 | 84.8 | 86.1 | 85.3 | 78.6 | 92.5 |
| 38 | 90.6 | 83.5 | 98.2 | 91.2 | 83.2 | 100.0 |
| 39 | 89.9 | 86.1 | 93.9 | 89.6 | 84.7 | 95.0 |
| 40 | 88.3 | 86.1 | 90.7 | 86.5 | 80.9 | 92.5 |
| 41 | 89.0 | 87.1 | 91.1 | 89.2 | 89.3 | 89.2 |
| 42 | 85.2 | 85.1 | 85.4 | 84.9 | 87.8 | 81.7 |
| 43 | 86.8 | 81.8 | 92.1 | 86.5 | 80.2 | 93.3 |
| 44 | 87.7 | 90.4 | 84.6 | 88.4 | 84.0 | 93.3 |
| 45 | 88.3 | 87.8 | 88.9 | 88.8 | 83.2 | 95.0 |
| 46 | 86.4 | 83.8 | 89.3 | 86.9 | 84.0 | 90.0 |
| 47 | 89.7 | 83.8 | 96.1 | 90.4 | 86.3 | 95.0 |
| 48 | 86.6 | 82.2 | 91.4 | 85.7 | 79.4 | 92.5 |
| 49 | 88.3 | 83.5 | 93.6 | 85.3 | 77.9 | 93.3 |
| 50 | 89.4 | 82.5 | 96.8 | 84.5 | 74.8 | 95.0 |
| 51 | 84.6 | 82.2 | 87.1 | 83.7 | 81.7 | 85.8 |
| 52 | 84.9 | 86.5 | 83.2 | 83.3 | 82.4 | 84.2 |
| 53 | 88.9 | 90.4 | 87.1 | 88.4 | 92.4 | 84.2 |
| 54 | 89.0 | 82.5 | 96.1 | 86.5 | 77.9 | 95.8 |
| 55 | 84.9 | 83.8 | 86.1 | 80.5 | 80.2 | 80.8 |
| 56 | 83.7 | 88.4 | 78.6 | 84.9 | 87.8 | 81.7 |
| 57 | 85.6 | 86.5 | 84.6 | 87.3 | 86.3 | 88.3 |
| 58 | 84.2 | 82.5 | 86.1 | 83.7 | 80.2 | 87.5 |
| 59 | 87.0 | 84.8 | 89.3 | 87.6 | 83.2 | 92.5 |
| 60 | 82.8 | 84.8 | 80.7 | 79.7 | 84.0 | 75.0 |
| 61 | 88.0 | 81.8 | 94.6 | 85.3 | 77.9 | 93.3 |
| 62 | 83.9 | 86.8 | 80.7 | 84.1 | 84.7 | 83.3 |
| 63 | 90.6 | 88.1 | 93.2 | 90.8 | 88.5 | 93.3 |
| 64 | 82.2 | 84.5 | 79.6 | 82.9 | 83.2 | 82.5 |
| 65 | 86.1 | 81.5 | 91.1 | 84.9 | 75.6 | 95.0 |
| 66 | 80.6 | 79.9 | 81.4 | 79.7 | 78.6 | 80.8 |
| 67 | 85.1 | 81.8 | 88.6 | 89.2 | 87.0 | 91.7 |
| 68 | 88.3 | 80.2 | 97.1 | 83.3 | 72.5 | 95.0 |
| 69 | 83.5 | 85.1 | 81.8 | 78.5 | 76.3 | 80.8 |
| 70 | 83.0 | 85.1 | 80.7 | 83.7 | 80.9 | 86.7 |
| 71 | 80.8 | 79.5 | 82.1 | 82.1 | 82.4 | 81.7 |
| 72 | 81.5 | 77.9 | 85.4 | 81.3 | 71.8 | 91.7 |
| 73 | 88.9 | 89.4 | 88.2 | 88.0 | 87.8 | 88.3 |
| 74 | 80.4 | 80.5 | 80.4 | 81.3 | 82.4 | 80.0 |
| 75 | 83.0 | 84.5 | 81.4 | 79.7 | 83.2 | 75.8 |
| 76 | 82.2 | 74.9 | 90.0 | 84.5 | 73.3 | 96.7 |
| 77 | 82.2 | 82.8 | 81.4 | 82.9 | 78.6 | 87.5 |
| 78 | 82.3 | 78.2 | 86.8 | 83.7 | 76.3 | 91.7 |
| 79 | 82.0 | 82.8 | 81.1 | 76.5 | 78.6 | 74.2 |
| 80 | 79.8 | 84.5 | 74.6 | 80.1 | 79.4 | 80.8 |
| 81 | 80.6 | 85.1 | 75.7 | 77.7 | 80.9 | 74.2 |
| 82 | 83.2 | 86.5 | 79.6 | 84.9 | 83.2 | 86.7 |
| 83 | 83.4 | 85.8 | 80.7 | 79.7 | 80.2 | 79.2 |
| 84 | 82.5 | 81.2 | 83.9 | 81.3 | 80.9 | 81.7 |
| 85 | 82.0 | 80.2 | 83.9 | 77.7 | 74.0 | 81.7 |
| 86 | 82.5 | 84.5 | 80.4 | 83.3 | 84.0 | 82.5 |
| 87 | 79.8 | 79.2 | 80.4 | 78.5 | 75.6 | 81.7 |
| 88 | 83.9 | 86.8 | 80.7 | 86.5 | 87.8 | 85.0 |
| 89 | 80.4 | 83.5 | 77.1 | 78.9 | 81.7 | 75.8 |
| 90 | 81.0 | 73.6 | 88.9 | 79.7 | 73.3 | 86.7 |
| 91 | 77.5 | 81.2 | 73.6 | 77.3 | 75.6 | 79.2 |
| 92 | 85.2 | 79.5 | 91.4 | 82.9 | 80.2 | 85.8 |
| 93 | 85.1 | 85.5 | 84.6 | 86.5 | 81.7 | 91.7 |
| 94 | 85.2 | 75.9 | 95.4 | 87.6 | 84.0 | 91.7 |
| 95 | 78.4 | 78.5 | 78.2 | 76.1 | 79.4 | 72.5 |
| 96 | 77.7 | 75.9 | 79.6 | 76.5 | 76.3 | 76.7 |
| 97 | 82.0 | 76.9 | 87.5 | 80.1 | 71.0 | 90.0 |
| 98 | 80.4 | 80.9 | 80.0 | 77.3 | 74.8 | 80.0 |
| 99 | 78.0 | 73.9 | 82.5 | 80.5 | 77.1 | 84.2 |
| 100 | 81.8 | 84.2 | 79.3 | 78.5 | 80.9 | 75.8 |
| 101 | 76.8 | 77.9 | 75.7 | 75.3 | 76.3 | 74.2 |
| 102 | 83.5 | 73.3 | 94.6 | 77.3 | 58.8 | 97.5 |
| 103 | 78.6 | 82.8 | 73.9 | 76.1 | 79.4 | 72.5 |
| 104 | 82.0 | 76.9 | 87.5 | 79.3 | 72.5 | 86.7 |
| 105 | 77.2 | 77.9 | 76.4 | 75.7 | 76.3 | 75.0 |
| 106 | 75.6 | 75.6 | 75.7 | 76.9 | 74.0 | 80.0 |
| 107 | 80.1 | 71.3 | 89.6 | 78.9 | 67.2 | 91.7 |
| 108 | 77.4 | 74.3 | 80.7 | 76.5 | 73.3 | 80.0 |
| 109 | 79.9 | 78.2 | 81.8 | 80.5 | 74.0 | 87.5 |
| 110 | 78.7 | 78.2 | 79.3 | 78.1 | 78.6 | 77.5 |
| 111 | 75.1 | 76.2 | 73.9 | 80.5 | 78.6 | 82.5 |
| 112 | 77.0 | 73.9 | 80.4 | 74.5 | 67.2 | 82.5 |
| 113 | 81.0 | 80.5 | 81.4 | 86.1 | 84.0 | 88.3 |
| 114 | 78.2 | 75.6 | 81.1 | 69.7 | 64.9 | 75.0 |
| 115 | 81.3 | 75.6 | 87.5 | 78.9 | 70.2 | 88.3 |
| 116 | 76.0 | 76.6 | 75.4 | 76.1 | 78.6 | 73.3 |
| 117 | 73.1 | 72.6 | 73.6 | 69.7 | 66.4 | 73.3 |
| 118 | 75.6 | 72.3 | 79.3 | 75.7 | 72.5 | 79.2 |
| 119 | 79.6 | 81.5 | 77.5 | 82.5 | 84.0 | 80.8 |
| 120 | 76.3 | 75.9 | 76.8 | 75.3 | 73.3 | 77.5 |
| 121 | 74.1 | 80.5 | 67.1 | 80.9 | 86.3 | 75.0 |
| 122 | 79.9 | 71.0 | 89.6 | 82.1 | 73.3 | 91.7 |
| 123 | 77.7 | 75.9 | 79.6 | 78.5 | 75.6 | 81.7 |
| 124 | 80.8 | 80.9 | 80.7 | 80.9 | 79.4 | 82.5 |
| 125 | 76.8 | 77.9 | 75.7 | 78.1 | 81.7 | 74.2 |
| 126 | 79.8 | 71.3 | 88.9 | 75.3 | 61.8 | 90.0 |
| 127 | 76.2 | 76.9 | 75.4 | 76.1 | 80.9 | 70.8 |
| 128 | 81.0 | 81.8 | 80.0 | 82.9 | 84.7 | 80.8 |
| 129 | 79.8 | 79.5 | 80.0 | 80.1 | 80.2 | 80.0 |
| 130 | 74.8 | 74.3 | 75.4 | 74.5 | 74.0 | 75.0 |
| 131 | 76.8 | 77.6 | 76.1 | 74.9 | 73.3 | 76.7 |
| 132 | 79.8 | 80.2 | 79.3 | 76.1 | 77.9 | 74.2 |
| 133 | 73.2 | 75.9 | 70.4 | 75.3 | 75.6 | 75.0 |
| 134 | 76.2 | 65.3 | 87.9 | 76.9 | 62.6 | 92.5 |
| 135 | 81.5 | 84.5 | 78.2 | 79.7 | 81.7 | 77.5 |
| 136 | 77.9 | 78.5 | 77.1 | 78.9 | 75.6 | 82.5 |
| 137 | 83.2 | 74.3 | 92.9 | 80.5 | 67.9 | 94.2 |
| 138 | 84.4 | 77.9 | 91.4 | 78.5 | 68.7 | 89.2 |
| 139 | 73.2 | 70.6 | 76.1 | 72.9 | 72.5 | 73.3 |
| 140 | 76.3 | 81.2 | 71.1 | 73.7 | 77.9 | 69.2 |
| 141 | 71.5 | 71.9 | 71.1 | 68.9 | 67.9 | 70.0 |
| 142 | 77.9 | 84.5 | 70.7 | 80.1 | 82.4 | 77.5 |
| 143 | 82.0 | 81.2 | 82.9 | 81.3 | 81.7 | 80.8 |
| 144 | 75.1 | 69.6 | 81.1 | 77.7 | 74.0 | 81.7 |
| 145 | 73.2 | 70.0 | 76.8 | 78.9 | 74.8 | 83.3 |
| 146 | 77.2 | 76.2 | 78.2 | 80.1 | 81.7 | 78.3 |
| 147 | 71.5 | 69.3 | 73.9 | 69.3 | 71.0 | 67.5 |
| 148 | 78.7 | 65.7 | 92.9 | 79.3 | 64.1 | 95.8 |
| 149 | 74.8 | 71.6 | 78.2 | 79.3 | 74.8 | 84.2 |
| 150 | 75.3 | 71.3 | 79.6 | 77.7 | 77.1 | 78.3 |
| 151 | 76.8 | 76.6 | 77.1 | 76.1 | 74.8 | 77.5 |
| 152 | 78.2 | 79.2 | 77.1 | 80.1 | 74.0 | 86.7 |
| 153 | 75.8 | 71.0 | 81.1 | 78.9 | 74.8 | 83.3 |
| 154 | 71.7 | 69.0 | 74.6 | 69.7 | 74.8 | 64.2 |
| 155 | 69.0 | 67.3 | 70.7 | 69.3 | 68.7 | 70.0 |
| 156 | 77.9 | 81.5 | 73.9 | 80.1 | 87.0 | 72.5 |
| 157 | 69.0 | 72.6 | 65.0 | 66.9 | 67.2 | 66.7 |
| 158 | 76.7 | 83.2 | 69.6 | 77.7 | 81.7 | 73.3 |
| 159 | 76.5 | 80.2 | 72.5 | 77.7 | 79.4 | 75.8 |
| 160 | 73.8 | 73.9 | 73.6 | 75.7 | 75.6 | 75.8 |
| 161 | 65.7 | 63.7 | 67.9 | 59.8 | 56.5 | 63.3 |
| 162 | 73.4 | 73.6 | 73.2 | 73.3 | 71.8 | 75.0 |
| 163 | 80.3 | 84.5 | 75.7 | 74.5 | 80.9 | 67.5 |
| 164 | 71.5 | 72.9 | 70.0 | 66.9 | 65.6 | 68.3 |
| 165 | 69.1 | 74.3 | 63.6 | 67.3 | 70.2 | 64.2 |
| 166 | 65.2 | 70.0 | 60.0 | 62.9 | 65.6 | 60.0 |
| 167 | 70.3 | 73.6 | 66.8 | 71.3 | 76.3 | 65.8 |
| 168 | 71.0 | 57.1 | 86.1 | 72.1 | 58.0 | 87.5 |
| 169 | 70.8 | 69.6 | 72.1 | 73.7 | 71.0 | 76.7 |
| 170 | 72.6 | 74.6 | 70.4 | 74.5 | 80.2 | 68.3 |
| 171 | 72.9 | 77.6 | 67.9 | 66.5 | 75.6 | 56.7 |
| 172 | 61.2 | 64.7 | 57.5 | 61.8 | 61.1 | 62.5 |
| 173 | 70.0 | 76.9 | 62.5 | 72.9 | 77.9 | 67.5 |
| 174 | 73.1 | 79.2 | 66.4 | 73.3 | 80.2 | 65.8 |
| 175 | 72.7 | 75.9 | 69.3 | 69.7 | 75.6 | 63.3 |
| 176 | 64.3 | 67.3 | 61.1 | 61.8 | 65.6 | 57.5 |
| 177 | 72.7 | 76.6 | 68.6 | 71.3 | 80.9 | 60.8 |
| 178 | 75.1 | 77.2 | 72.9 | 73.3 | 78.6 | 67.5 |
| 179 | 73.8 | 78.2 | 68.9 | 76.9 | 80.9 | 72.5 |
| 180 | 70.7 | 77.6 | 63.2 | 68.1 | 65.6 | 70.8 |
| 181 | 65.9 | 66.7 | 65.0 | 70.1 | 65.6 | 75.0 |
| 182 | 67.2 | 61.7 | 73.2 | 68.1 | 58.8 | 78.3 |
| 183 | 66.7 | 72.9 | 60.0 | 66.5 | 67.9 | 65.0 |
| 184 | 69.3 | 73.6 | 64.6 | 68.9 | 70.2 | 67.5 |
| 185 | 68.4 | 74.9 | 61.4 | 68.5 | 73.3 | 63.3 |
| 186 | 68.8 | 73.6 | 63.6 | 71.3 | 73.3 | 69.2 |
| 187 | 68.8 | 75.6 | 61.4 | 71.3 | 83.2 | 58.3 |
| 188 | 66.0 | 54.8 | 78.2 | 63.3 | 48.1 | 80.0 |
| 189 | 69.6 | 69.6 | 69.6 | 69.7 | 69.5 | 70.0 |
| 190 | 64.0 | 72.6 | 54.6 | 68.5 | 76.3 | 60.0 |
| 191 | 67.9 | 74.3 | 61.1 | 68.5 | 76.3 | 60.0 |
| 192 | 64.3 | 60.7 | 68.2 | 64.5 | 55.7 | 74.2 |
| 193 | 63.5 | 64.7 | 62.1 | 60.6 | 58.0 | 63.3 |
| 194 | 64.0 | 64.0 | 63.9 | 66.5 | 61.8 | 71.7 |
| 195 | 67.6 | 67.3 | 67.9 | 62.2 | 56.5 | 68.3 |
| 196 | 66.7 | 71.6 | 61.4 | 71.3 | 73.3 | 69.2 |
| 197 | 63.0 | 58.7 | 67.5 | 62.2 | 55.0 | 70.0 |
| 198 | 63.0 | 69.3 | 56.1 | 69.7 | 74.8 | 64.2 |
| 199 | 63.5 | 70.6 | 55.7 | 68.1 | 68.7 | 67.5 |
| 200 | 62.4 | 66.0 | 58.6 | 60.2 | 59.5 | 60.8 |
| 201 | 64.7 | 68.3 | 60.7 | 64.1 | 63.4 | 65.0 |
| 202 | 58.3 | 54.1 | 62.9 | 59.4 | 54.2 | 65.0 |
| 203 | 57.1 | 68.6 | 44.6 | 62.9 | 73.3 | 51.7 |
| 248 | 95.5 | 95.7 | 95.4 | 97.6 | 99.2 | 95.8 |
| 249 | 94.3 | 90.8 | 98.2 | 93.2 | 87.8 | 99.2 |
| 250 | 93.1 | 93.7 | 92.5 | 94.8 | 93.1 | 96.7 |
| 251 | 91.3 | 88.4 | 94.3 | 93.2 | 90.8 | 95.8 |
| 252 | 90.2 | 89.1 | 91.4 | 88.4 | 83.2 | 94.2 |
| 253 | 89.5 | 87.5 | 91.8 | 90.0 | 87.8 | 92.5 |
| 254 | 84.9 | 83.8 | 86.1 | 88.8 | 89.3 | 88.3 |
| 255 | 90.2 | 84.8 | 96.1 | 90.4 | 82.4 | 99.2 |
| 256 | 87.3 | 91.4 | 82.9 | 92.4 | 94.7 | 90.0 |
| 257 | 86.1 | 84.5 | 87.9 | 83.7 | 80.2 | 87.5 |
| 258 | 87.8 | 82.8 | 93.2 | 88.0 | 84.7 | 91.7 |
| 259 | 93.8 | 89.4 | 98.6 | 93.2 | 90.8 | 95.8 |
| 260 | 84.6 | 94.4 | 73.9 | 85.7 | 95.4 | 75.0 |
| 261 | 82.2 | 84.5 | 79.6 | 84.9 | 80.9 | 89.2 |
| 262 | 80.4 | 82.5 | 78.2 | 79.3 | 81.7 | 76.7 |
| 263 | 80.1 | 73.3 | 87.5 | 74.5 | 65.6 | 84.2 |
| 264 | 79.2 | 83.5 | 74.6 | 76.9 | 80.2 | 73.3 |
| 265 | 82.2 | 81.8 | 82.5 | 79.3 | 74.8 | 84.2 |
| 266 | 77.0 | 77.6 | 76.4 | 76.9 | 77.9 | 75.8 |
| 267 | 70.2 | 73.9 | 66.1 | 72.5 | 77.1 | 67.5 |
| 268 | 65.5 | 63.0 | 68.2 | 63.3 | 61.8 | 65.0 |

TABLE 4

| SEQ | | Constant |
|---|---|---|
| ID NO. | Coefficient | term |
| 1 | 2.123 | 16.461 |
| 2 | 1.384 | 9.575 |
| 3 | 4.869 | 56.826 |
| 4 | 1.396 | 17.610 |
| 5 | 3.373 | 29.135 |
| 6 | 2.664 | 32.360 |
| 7 | 3.268 | 38.312 |
| 8 | 1.163 | 7.999 |
| 9 | 1.368 | 9.338 |
| 10 | 1.159 | 7.999 |
| 11 | 1.289 | 15.195 |
| 12 | 2.319 | 25.799 |
| 13 | 1.757 | 12.187 |
| 14 | 0.879 | 4.347 |
| 15 | 1.520 | 15.872 |
| 16 | 1.908 | 15.742 |
| 17 | 1.661 | 11.329 |
| 18 | 1.089 | 9.869 |
| 19 | 1.629 | 18.376 |
| 20 | 0.893 | 10.428 |
| 21 | 1.170 | 6.573 |
| 22 | 2.781 | 23.633 |
| 23 | 1.499 | 9.978 |
| 24 | 3.569 | 39.272 |
| 25 | 1.449 | 12.427 |
| 26 | 1.356 | 12.568 |
| 27 | 2.427 | 20.795 |
| 28 | 2.016 | 22.808 |
| 29 | 0.705 | 3.606 |
| 30 | 2.106 | 15.535 |
| 31 | 4.170 | 38.602 |
| 32 | 1.077 | 6.983 |
| 33 | 2.681 | 29.153 |
| 34 | 2.551 | 22.591 |
| 35 | 2.124 | 25.916 |
| 36 | 2.250 | 22.402 |
| 37 | 2.375 | 24.609 |
| 38 | 2.812 | 28.525 |
| 39 | 1.058 | 5.677 |
| 40 | 1.194 | 9.320 |
| 41 | 1.227 | 7.549 |
| 42 | 1.044 | 10.432 |
| 43 | 2.130 | 16.511 |
| 44 | 2.163 | 14.618 |
| 45 | 1.545 | 10.595 |
| 46 | 3.952 | 49.258 |
| 47 | 1.061 | 7.156 |
| 48 | 2.814 | 27.273 |
| 49 | 2.491 | 31.077 |
| 50 | 1.162 | 7.912 |
| 51 | 1.595 | 13.403 |
| 52 | 2.381 | 19.963 |
| 53 | 1.479 | 9.318 |
| 54 | 3.435 | 36.734 |
| 55 | 0.662 | 4.595 |
| 56 | 2.481 | 21.976 |
| 57 | 1.778 | 12.553 |
| 58 | 3.391 | 35.584 |
| 59 | 1.051 | 6.829 |
| 60 | 1.161 | 9.868 |
| 61 | 2.574 | 32.431 |
| 62 | 1.991 | 14.191 |
| 63 | 1.457 | 8.857 |
| 64 | 3.740 | 37.811 |
| 65 | 1.321 | 9.518 |
| 66 | 0.742 | 7.056 |
| 67 | 0.934 | 5.001 |
| 68 | 2.165 | 18.275 |
| 69 | 0.924 | 7.932 |
| 70 | 2.595 | 23.645 |
| 71 | 1.101 | 8.477 |
| 72 | 3.198 | 31.760 |
| 73 | 2.473 | 16.893 |
| 74 | 1.157 | 9.485 |
| 75 | 1.585 | 11.119 |
| 76 | 1.212 | 8.366 |
| 77 | 1.082 | 8.518 |
| 78 | 1.893 | 14.755 |
| 79 | 2.213 | 18.358 |
| 80 | 1.235 | 8.533 |
| 81 | 1.182 | 8.574 |
| 82 | 1.723 | 11.225 |
| 83 | 0.541 | 3.403 |
| 84 | 1.024 | 6.335 |
| 85 | 1.054 | 9.531 |
| 86 | 1.358 | 8.692 |
| 87 | 0.647 | 4.678 |
| 88 | 1.240 | 7.612 |
| 89 | 1.568 | 9.448 |
| 90 | 1.250 | 9.664 |
| 91 | 1.205 | 7.983 |
| 92 | 1.149 | 6.022 |
| 93 | 1.556 | 9.561 |
| 94 | 1.388 | 9.969 |
| 95 | 0.968 | 7.612 |
| 96 | 2.008 | 18.975 |
| 97 | 1.616 | 11.918 |
| 98 | 0.618 | 4.008 |
| 99 | 0.445 | 3.689 |
| 100 | 1.881 | 12.492 |
| 101 | 1.709 | 15.312 |
| 102 | 1.633 | 20.622 |
| 103 | 0.498 | 2.830 |
| 104 | 2.266 | 28.614 |
| 105 | 0.604 | 4.325 |
| 106 | 1.675 | 12.286 |
| 107 | 1.282 | 8.167 |
| 108 | 2.394 | 23.435 |
| 109 | 1.053 | 8.539 |
| 110 | 0.985 | 6.096 |
| 111 | 1.694 | 14.189 |
| 112 | 0.931 | 6.212 |
| 113 | 1.531 | 10.326 |
| 114 | 2.056 | 16.586 |
| 115 | 1.298 | 11.492 |
| 116 | 0.569 | 3.293 |
| 117 | 2.355 | 27.797 |
| 118 | 1.646 | 15.659 |
| 119 | 1.815 | 11.796 |
| 120 | 1.022 | 7.122 |
| 121 | 1.426 | 18.104 |
| 122 | 0.629 | 3.413 |
| 123 | 2.416 | 32.426 |
| 124 | 1.319 | 7.733 |
| 125 | 0.771 | 5.365 |
| 126 | 1.242 | 8.113 |
| 127 | 0.719 | 3.627 |
| 128 | 1.421 | 7.974 |
| 129 | 1.134 | 6.426 |
| 130 | 2.258 | 18.373 |
| 131 | 1.364 | 10.682 |
| 132 | 1.137 | 6.086 |
| 133 | 1.921 | 19.578 |
| 134 | 1.217 | 7.932 |
| 135 | 1.182 | 6.733 |
| 136 | 1.373 | 10.118 |
| 137 | 1.550 | 11.154 |
| 138 | 0.848 | 5.153 |
| 139 | 2.009 | 21.173 |
| 140 | 1.277 | 9.081 |
| 141 | 0.676 | 3.802 |
| 142 | 1.076 | 6.019 |
| 143 | 1.466 | 8.667 |
| 144 | 1.735 | 23.462 |
| 145 | 1.904 | 15.630 |
| 146 | 1.196 | 7.706 |
| 147 | 1.661 | 12.333 |
| 148 | 1.332 | 9.486 |
| 149 | 1.902 | 14.524 |
| 150 | 1.901 | 15.043 |
| 151 | 2.365 | 17.993 |
| 152 | 1.519 | 8.468 |
| 153 | 0.925 | 5.559 |
| 154 | 1.424 | 14.174 |
| 155 | 1.445 | 11.773 |
| 156 | 1.406 | 7.825 |
| 157 | 1.499 | 11.695 |
| 158 | 1.306 | 8.480 |
| 159 | 1.422 | 8.864 |
| 160 | 1.166 | 7.753 |
| 161 | 0.986 | 7.102 |
| 162 | 1.026 | 6.301 |
| 163 | 1.550 | 10.712 |
| 164 | 0.695 | 3.993 |
| 165 | 1.148 | 7.715 |
| 166 | 1.311 | 10.092 |
| 167 | 1.170 | 6.320 |
| 168 | 1.097 | 6.199 |
| 169 | 1.178 | 7.806 |
| 170 | 1.298 | 7.638 |
| 171 | 1.404 | 8.033 |
| 172 | 1.291 | 9.864 |
| 173 | 1.190 | 7.474 |
| 174 | 1.278 | 7.428 |
| 175 | 1.277 | 8.008 |
| 176 | 1.228 | 10.468 |
| 177 | 1.309 | 7.689 |
| 178 | 1.543 | 8.824 |
| 179 | 1.442 | 8.131 |
| 180 | 1.429 | 8.934 |
| 181 | 1.530 | 10.398 |
| 182 | 0.834 | 4.706 |
| 183 | 1.229 | 7.422 |
| 184 | 1.446 | 9.586 |
| 185 | 1.379 | 8.242 |
| 186 | 1.046 | 5.969 |
| 187 | 1.330 | 7.589 |
| 188 | 1.277 | 7.572 |
| 189 | 1.307 | 8.341 |
| 190 | 1.194 | 7.388 |
| 191 | 0.838 | 4.687 |
| 192 | 1.219 | 7.400 |
| 193 | 1.091 | 6.825 |
| 194 | 1.007 | 8.299 |
| 195 | 1.271 | 9.982 |
| 196 | 1.044 | 7.688 |
| 197 | 1.019 | 6.103 |
| 198 | 1.082 | 7.372 |
| 199 | 1.068 | 6.872 |
| 200 | 1.129 | 8.982 |
| 201 | 1.114 | 7.351 |
| 202 | 1.082 | 6.606 |
| 203 | 0.916 | 5.481 |
| 248 | 1.159 | 6.820 |
| 249 | 1.888 | 21.584 |
| 250 | 4.673 | 53.498 |
| 251 | 1.396 | 12.941 |
| 252 | 1.537 | 16.846 |
| 253 | 0.902 | 4.750 |
| 254 | 0.576 | 2.111 |
| 255 | 1.989 | 18.569 |
| 256 | 2.231 | 17.384 |
| 257 | 0.430 | 2.206 |
| 258 | 1.303 | 11.311 |
| 259 | 1.409 | 9.381 |
| 260 | 0.454 | 3.040 |
| 261 | 1.401 | 9.414 |
| 262 | 1.566 | 13.242 |
| 263 | 1.007 | 6.487 |
| 264 | 0.430 | 2.247 |
| 265 | 1.145 | 6.375 |
| 266 | 0.721 | 3.898 |
| 267 | 0.914 | 6.308 |
| 268 | 1.098 | 8.628 |

TABLE 5

| | Training cohort | | | Validation cohort | | |
|---|---|---|---|---|---|---|
| | Accuracy | Sensitivity | Specificity | Accuracy | Sensitivity | |
| SEQ ID NO. | (%) | (%) | (%) | (%) | (%) | Specificity (%) |
| 1_3 | 99.5 | 100.0 | 98.9 | 100.0 | 100.0 | 100.0 |
| 2_3 | 98.3 | 97.0 | 99.6 | 98.8 | 97.7 | 100.0 |
| 1_3 | 99.5 | 100.0 | 98.9 | 100.0 | 100.0 | 100.0 |
| 3_4 | 99.1 | 99.0 | 99.3 | 99.6 | 99.2 | 100.0 |
| 3_5 | 99.5 | 100.0 | 98.9 | 100.0 | 100.0 | 100.0 |
| 3_6 | 99.1 | 99.0 | 99.3 | 99.6 | 99.2 | 100.0 |
| 3_7 | 98.3 | 97.0 | 99.6 | 99.6 | 99.2 | 100.0 |
| 6_8 | 99.3 | 98.7 | 100.0 | 99.2 | 98.5 | 100.0 |
| 3_9 | 98.8 | 98.7 | 98.9 | 99.2 | 98.5 | 100.0 |
| 248_10 | 99.3 | 100.0 | 98.6 | 100.0 | 100.0 | 100.0 |
| 11_18 | 99.7 | 99.3 | 100.0 | 100.0 | 100.0 | 100.0 |
| 3_12 | 98.8 | 98.3 | 99.3 | 99.2 | 98.5 | 100.0 |
| 3_13 | 99.0 | 98.7 | 99.3 | 99.6 | 100.0 | 99.2 |
| 14_16 | 97.6 | 98.0 | 97.1 | 100.0 | 100.0 | 100.0 |
| 6_15 | 97.8 | 99.7 | 95.7 | 99.2 | 99.2 | 99.2 |
| 248_16 | 97.6 | 97.7 | 97.5 | 100.0 | 100.0 | 100.0 |
| 6_17 | 98.8 | 98.7 | 98.9 | 99.2 | 98.5 | 100.0 |
| 248_18 | 97.9 | 97.0 | 98.9 | 100.0 | 100.0 | 100.0 |
| 3_19 | 98.8 | 98.3 | 99.3 | 99.2 | 98.5 | 100.0 |
| 3_20 | 98.3 | 97.7 | 98.9 | 99.2 | 98.5 | 100.0 |
| 3_21 | 98.5 | 98.0 | 98.9 | 98.8 | 98.5 | 99.2 |
| 3_22 | 98.8 | 98.7 | 98.9 | 99.2 | 99.2 | 99.2 |
| 3_23 | 99.1 | 99.0 | 99.3 | 99.6 | 100.0 | 99.2 |
| 3_24 | 99.0 | 99.0 | 98.9 | 99.2 | 99.2 | 99.2 |
| 1_25 | 99.0 | 99.3 | 98.6 | 99.2 | 98.5 | 100.0 |
| 3_26 | 98.3 | 97.4 | 99.3 | 98.4 | 97.7 | 99.2 |
| 3_27 | 99.0 | 98.7 | 99.3 | 99.6 | 99.2 | 100.0 |
| 1_28 | 97.9 | 99.0 | 96.8 | 99.2 | 99.2 | 99.2 |
| 3_29 | 98.8 | 98.3 | 99.3 | 99.6 | 99.2 | 100.0 |
| 16_30 | 97.9 | 98.0 | 97.9 | 99.2 | 98.5 | 100.0 |
| 3_31 | 98.3 | 97.4 | 99.3 | 99.6 | 99.2 | 100.0 |
| 3_32 | 98.8 | 98.3 | 99.3 | 99.2 | 98.5 | 100.0 |
| 3_33 | 97.6 | 96.4 | 98.9 | 98.8 | 97.7 | 100.0 |
| 6_34 | 98.3 | 99.7 | 96.8 | 99.2 | 99.2 | 99.2 |
| 3_35 | 98.8 | 98.3 | 99.3 | 98.8 | 97.7 | 100.0 |
| 3_36 | 98.5 | 98.0 | 98.9 | 99.2 | 99.2 | 99.2 |
| 3_37 | 99.0 | 99.0 | 98.9 | 99.6 | 99.2 | 100.0 |
| 11_38 | 99.7 | 99.3 | 100.0 | 99.2 | 98.5 | 100.0 |
| 3_39 | 97.6 | 96.0 | 99.3 | 99.2 | 98.5 | 100.0 |
| 3_40 | 98.5 | 98.0 | 98.9 | 99.2 | 98.5 | 100.0 |
| 3_41 | 98.1 | 97.4 | 98.9 | 98.8 | 98.5 | 99.2 |
| 3_42 | 97.6 | 96.7 | 98.6 | 99.2 | 98.5 | 100.0 |
| 3_43 | 98.1 | 97.4 | 98.9 | 99.2 | 99.2 | 99.2 |
| 3_44 | 98.6 | 98.3 | 98.9 | 98.8 | 98.5 | 99.2 |
| 3_45 | 99.0 | 98.7 | 99.3 | 99.2 | 98.5 | 100.0 |
| 3_46 | 98.5 | 97.7 | 99.3 | 98.8 | 97.7 | 100.0 |
| 248_47 | 95.2 | 93.7 | 96.8 | 99.2 | 99.2 | 99.2 |
| 6_48 | 98.1 | 99.0 | 97.1 | 98.8 | 98.5 | 99.2 |
| 249_49 | 99.3 | 99.0 | 99.6 | 99.2 | 98.5 | 100.0 |
| 3_50 | 98.8 | 98.7 | 98.9 | 98.8 | 97.7 | 100.0 |
| 3_51 | 97.8 | 97.0 | 98.6 | 98.8 | 97.7 | 100.0 |
| 3_52 | 99.0 | 99.0 | 98.9 | 98.8 | 98.5 | 99.2 |
| 16_53 | 97.6 | 96.7 | 98.6 | 99.6 | 99.2 | 100.0 |
| 3_54 | 97.4 | 96.0 | 98.9 | 99.6 | 99.2 | 100.0 |
| 7_55 | 96.2 | 93.1 | 99.6 | 97.2 | 94.7 | 100.0 |
| 248_56 | 95.9 | 95.7 | 96.1 | 99.6 | 99.2 | 100.0 |
| 3_57 | 98.6 | 98.0 | 99.3 | 99.2 | 99.2 | 99.2 |
| 249_58 | 99.5 | 100.0 | 98.9 | 99.6 | 100.0 | 99.2 |
| 3_59 | 97.6 | 96.4 | 98.9 | 98.8 | 98.5 | 99.2 |
| 1_60 | 98.1 | 98.7 | 97.5 | 98.8 | 97.7 | 100.0 |
| 249_61 | 99.1 | 98.7 | 99.6 | 98.8 | 97.7 | 100.0 |
| 3_62 | 98.8 | 98.7 | 98.9 | 99.2 | 99.2 | 99.2 |
| 3_63 | 97.6 | 96.7 | 98.6 | 99.2 | 99.2 | 99.2 |
| 248_64 | 96.6 | 95.4 | 97.9 | 98.0 | 97.7 | 98.3 |
| 3_65 | 99.0 | 98.7 | 99.3 | 99.2 | 98.5 | 100.0 |
| 3_66 | 98.3 | 97.7 | 98.9 | 99.2 | 99.2 | 99.2 |
| 3_67 | 98.1 | 97.4 | 98.9 | 98.8 | 98.5 | 99.2 |
| 16_68 | 99.3 | 98.7 | 100.0 | 99.6 | 99.2 | 100.0 |
| 3_69 | 98.3 | 97.7 | 98.9 | 98.4 | 97.7 | 99.2 |
| 3_70 | 99.0 | 99.3 | 98.6 | 99.2 | 99.2 | 99.2 |
| 248_71 | 95.7 | 95.4 | 96.1 | 98.8 | 100.0 | 97.5 |
| 3_72 | 98.6 | 98.3 | 98.9 | 99.6 | 99.2 | 100.0 |
| 3_73 | 97.8 | 97.4 | 98.2 | 98.8 | 98.5 | 99.2 |
| 3_74 | 98.1 | 97.4 | 98.9 | 99.2 | 99.2 | 99.2 |
| 1_75 | 97.3 | 98.7 | 95.7 | 98.8 | 98.5 | 99.2 |
| 3_76 | 98.8 | 98.3 | 99.3 | 98.4 | 96.9 | 100.0 |
| 3_77 | 98.1 | 97.4 | 98.9 | 99.2 | 99.2 | 99.2 |
| 3_78 | 97.9 | 97.0 | 98.9 | 99.6 | 99.2 | 100.0 |
| 1_79 | 97.9 | 99.0 | 96.8 | 99.6 | 99.2 | 100.0 |
| 18_80 | 99.0 | 98.3 | 99.6 | 99.6 | 99.2 | 100.0 |
| 3_81 | 97.9 | 97.4 | 98.6 | 98.4 | 98.5 | 98.3 |
| 3_82 | 98.5 | 98.0 | 98.9 | 98.8 | 98.5 | 99.2 |
| 3_83 | 97.9 | 97.0 | 98.9 | 99.2 | 98.5 | 100.0 |
| 3_84 | 97.4 | 96.4 | 98.6 | 98.4 | 97.7 | 99.2 |
| 10_85 | 98.1 | 97.4 | 98.9 | 98.8 | 97.7 | 100.0 |
| 16_86 | 98.5 | 97.7 | 99.3 | 99.6 | 99.2 | 100.0 |
| 16_87 | 97.1 | 95.4 | 98.9 | 99.2 | 99.2 | 99.2 |
| 3_88 | 98.6 | 98.0 | 99.3 | 98.8 | 98.5 | 99.2 |
| 3_89 | 98.3 | 97.7 | 98.9 | 98.8 | 98.5 | 99.2 |
| 3_90 | 97.8 | 97.0 | 98.6 | 98.8 | 98.5 | 99.2 |
| 248_91 | 95.7 | 95.4 | 96.1 | 98.0 | 99.2 | 96.7 |
| 249_92 | 97.6 | 97.0 | 98.2 | 99.2 | 98.5 | 100.0 |
| 3_93 | 97.9 | 97.0 | 98.9 | 98.8 | 98.5 | 99.2 |
| 3_94 | 98.1 | 97.4 | 98.9 | 99.2 | 98.5 | 100.0 |
| 3_95 | 97.4 | 96.4 | 98.6 | 98.4 | 97.7 | 99.2 |
| 1_96 | 97.3 | 99.0 | 95.4 | 98.4 | 97.7 | 99.2 |
| 3_97 | 98.6 | 98.3 | 98.9 | 99.2 | 98.5 | 100.0 |
| 16_98 | 96.2 | 95.0 | 97.5 | 99.2 | 98.5 | 100.0 |
| 6_99 | 97.9 | 97.4 | 98.6 | 98.8 | 98.5 | 99.2 |
| 10_100 | 98.6 | 97.4 | 100.0 | 99.2 | 98.5 | 100.0 |
| 1_101 | 97.6 | 99.0 | 96.1 | 99.2 | 99.2 | 99.2 |
| 6_102 | 98.8 | 99.0 | 98.6 | 98.8 | 97.7 | 100.0 |
| 3_103 | 98.1 | 97.4 | 98.9 | 98.4 | 98.5 | 98.3 |
| 249_104 | 99.7 | 99.7 | 99.6 | 99.6 | 99.2 | 100.0 |
| 16_105 | 96.1 | 95.0 | 97.1 | 100.0 | 100.0 | 100.0 |
| 3_106 | 98.3 | 97.7 | 98.9 | 99.2 | 99.2 | 99.2 |
| 3_107 | 98.6 | 98.3 | 98.9 | 98.8 | 97.7 | 100.0 |
| 7_108 | 96.4 | 93.4 | 99.6 | 98.4 | 96.9 | 100.0 |
| 3_109 | 97.9 | 97.0 | 98.9 | 98.8 | 98.5 | 99.2 |
| 10_110 | 99.1 | 99.0 | 99.3 | 98.8 | 97.7 | 100.0 |
| 248_111 | 95.5 | 95.7 | 95.4 | 98.4 | 99.2 | 97.5 |
| 3_112 | 98.3 | 97.7 | 98.9 | 99.2 | 98.5 | 100.0 |
| 248_113 | 95.0 | 94.4 | 95.7 | 98.8 | 100.0 | 97.5 |
| 249_114 | 99.3 | 99.3 | 99.3 | 99.6 | 99.2 | 100.0 |
| 248_115 | 96.4 | 96.4 | 96.4 | 99.6 | 100.0 | 99.2 |
| 3_116 | 97.8 | 96.7 | 98.9 | 98.8 | 98.5 | 99.2 |
| 249_117 | 98.8 | 100.0 | 97.5 | 99.6 | 100.0 | 99.2 |
| 3_118 | 97.6 | 96.7 | 98.6 | 98.4 | 96.9 | 100.0 |
| 3_119 | 97.9 | 97.4 | 98.6 | 99.2 | 98.5 | 100.0 |
| 3_120 | 98.5 | 98.0 | 98.9 | 98.8 | 97.7 | 100.0 |
| 3_121 | 98.6 | 98.3 | 98.9 | 99.2 | 99.2 | 99.2 |
| 3_122 | 97.6 | 96.7 | 98.6 | 98.4 | 97.7 | 99.2 |
| 3_123 | 97.4 | 96.0 | 98.9 | 98.8 | 98.5 | 99.2 |
| 3_124 | 97.4 | 96.4 | 98.6 | 98.8 | 98.5 | 99.2 |
| 3_125 | 97.6 | 96.4 | 98.9 | 98.4 | 97.7 | 99.2 |
| 248_126 | 96.4 | 96.4 | 96.4 | 98.8 | 99.2 | 98.3 |
| 10_127 | 97.9 | 97.0 | 98.9 | 99.2 | 98.5 | 100.0 |
| 248_128 | 95.0 | 94.7 | 95.4 | 98.0 | 98.5 | 97.5 |
| 16_129 | 96.6 | 93.7 | 99.6 | 98.8 | 98.5 | 99.2 |
| 3_130 | 98.3 | 97.4 | 99.3 | 98.4 | 97.7 | 99.2 |
| 3_131 | 98.5 | 98.0 | 98.9 | 98.8 | 98.5 | 99.2 |
| 3_132 | 97.8 | 97.0 | 98.6 | 98.4 | 97.7 | 99.2 |
| 1_133 | 98.1 | 99.0 | 97.1 | 99.6 | 99.2 | 100.0 |
| 3_134 | 98.3 | 97.7 | 98.9 | 98.8 | 97.7 | 100.0 |
| 3_135 | 97.8 | 96.7 | 98.9 | 98.4 | 97.7 | 99.2 |
| 10_136 | 98.8 | 98.3 | 99.3 | 98.8 | 97.7 | 100.0 |
| 3_137 | 98.5 | 98.0 | 98.9 | 99.2 | 98.5 | 100.0 |
| 3_138 | 97.3 | 96.0 | 98.6 | 98.4 | 97.7 | 99.2 |
| 249_139 | 96.6 | 94.1 | 99.3 | 98.8 | 97.7 | 100.0 |
| 10_140 | 97.4 | 97.4 | 97.5 | 99.2 | 98.5 | 100.0 |
| 3_141 | 97.9 | 97.0 | 98.9 | 98.8 | 97.7 | 100.0 |
| 3_142 | 97.8 | 97.0 | 98.6 | 99.2 | 98.5 | 100.0 |
| 3_143 | 97.8 | 97.0 | 98.6 | 98.4 | 97.7 | 99.2 |
| 19_144 | 97.8 | 97.4 | 98.2 | 99.2 | 98.5 | 100.0 |
| 3_145 | 97.8 | 96.7 | 98.9 | 99.2 | 98.5 | 100.0 |
| 3_146 | 97.6 | 96.7 | 98.6 | 98.4 | 97.7 | 99.2 |
| 3_147 | 97.8 | 97.0 | 98.6 | 98.4 | 97.7 | 99.2 |
| 248_148 | 95.5 | 94.7 | 96.4 | 98.0 | 98.5 | 97.5 |
| 3_149 | 97.8 | 96.7 | 98.9 | 98.8 | 98.5 | 99.2 |
| 3_150 | 98.3 | 97.7 | 98.9 | 99.2 | 99.2 | 99.2 |
| 3_151 | 97.4 | 96.4 | 98.6 | 98.8 | 98.5 | 99.2 |
| 3_152 | 97.4 | 96.4 | 98.6 | 98.8 | 98.5 | 99.2 |
| 3_153 | 97.4 | 96.4 | 98.6 | 98.4 | 97.7 | 99.2 |
| 3_154 | 97.8 | 96.7 | 98.9 | 99.2 | 98.5 | 100.0 |
| 3_155 | 98.8 | 98.3 | 99.3 | 98.8 | 98.5 | 99.2 |
| 10_156 | 97.8 | 97.0 | 98.6 | 98.8 | 97.7 | 100.0 |
| 3_157 | 97.9 | 96.7 | 99.3 | 98.4 | 97.7 | 99.2 |
| 248_158 | 95.9 | 96.0 | 95.7 | 98.4 | 100.0 | 96.7 |
| 10_159 | 98.8 | 99.3 | 98.2 | 98.4 | 96.9 | 100.0 |
| 10_160 | 98.5 | 97.4 | 99.6 | 99.6 | 99.2 | 100.0 |
| 6_161 | 97.8 | 97.0 | 98.6 | 98.4 | 97.7 | 99.2 |
| 16_162 | 97.4 | 96.0 | 98.9 | 99.6 | 100.0 | 99.2 |
| 3_163 | 97.8 | 97.0 | 98.6 | 98.4 | 97.7 | 99.2 |
| 248_164 | 95.4 | 96.0 | 94.6 | 98.8 | 100.0 | 97.5 |
| 248_165 | 95.2 | 95.0 | 95.4 | 98.0 | 99.2 | 96.7 |
| 3_166 | 98.3 | 97.7 | 98.9 | 98.8 | 97.7 | 100.0 |
| 3_167 | 98.1 | 97.7 | 98.6 | 98.8 | 98.5 | 99.2 |
| 248_168 | 95.9 | 95.4 | 96.4 | 98.0 | 98.5 | 97.5 |
| 3_169 | 97.6 | 96.7 | 98.6 | 98.4 | 97.7 | 99.2 |
| 3_170 | 97.9 | 97.4 | 98.6 | 98.4 | 97.7 | 99.2 |
| 3_171 | 97.3 | 96.0 | 98.6 | 98.4 | 97.7 | 99.2 |
| 3_172 | 97.6 | 96.7 | 98.6 | 98.4 | 97.7 | 99.2 |
| 3_173 | 97.4 | 96.0 | 98.9 | 98.4 | 97.7 | 99.2 |
| 3_174 | 97.6 | 96.7 | 98.6 | 98.8 | 98.5 | 99.2 |
| 3_175 | 97.4 | 96.4 | 98.6 | 98.4 | 97.7 | 99.2 |
| 3_176 | 98.3 | 97.7 | 98.9 | 98.8 | 97.7 | 100.0 |
| 3_177 | 97.3 | 96.0 | 98.6 | 98.4 | 97.7 | 99.2 |
| 6_178 | 97.3 | 98.0 | 96.4 | 98.4 | 98.5 | 98.3 |
| 3_179 | 98.3 | 98.0 | 98.6 | 98.4 | 97.7 | 99.2 |
| 3_180 | 97.6 | 96.7 | 98.6 | 98.4 | 97.7 | 99.2 |
| 3_181 | 97.8 | 96.7 | 98.9 | 98.4 | 97.7 | 99.2 |
| 3_182 | 98.5 | 98.3 | 98.6 | 98.8 | 97.7 | 100.0 |
| 3_183 | 97.6 | 96.7 | 98.6 | 98.4 | 97.7 | 99.2 |
| 3_184 | 97.6 | 96.7 | 98.6 | 98.4 | 97.7 | 99.2 |
| 3_185 | 97.6 | 96.7 | 98.6 | 98.4 | 97.7 | 99.2 |
| 18_186 | 95.7 | 93.1 | 98.6 | 95.6 | 93.9 | 97.5 |
| 3_187 | 97.6 | 96.7 | 98.6 | 98.8 | 97.7 | 100.0 |
| 1_188 | 97.3 | 98.7 | 95.7 | 98.0 | 97.7 | 98.3 |
| 10_189 | 98.6 | 98.0 | 99.3 | 98.4 | 97.7 | 99.2 |
| 3_190 | 97.6 | 96.7 | 98.6 | 98.4 | 97.7 | 99.2 |
| 3_191 | 97.4 | 96.4 | 98.6 | 98.4 | 97.7 | 99.2 |
| 6_192 | 96.6 | 97.4 | 95.7 | 98.0 | 98.5 | 97.5 |
| 3_193 | 98.3 | 98.0 | 98.6 | 98.8 | 98.5 | 99.2 |
| 3_194 | 97.6 | 96.7 | 98.6 | 98.4 | 97.7 | 99.2 |
| 3_195 | 97.6 | 96.7 | 98.6 | 98.4 | 97.7 | 99.2 |
| 3_196 | 97.6 | 96.7 | 98.6 | 98.4 | 97.7 | 99.2 |
| 3_197 | 97.9 | 97.0 | 98.9 | 98.4 | 97.7 | 99.2 |
| 10_198 | 98.5 | 99.0 | 97.9 | 98.8 | 98.5 | 99.2 |
| 3_199 | 97.6 | 96.7 | 98.6 | 98.8 | 98.5 | 99.2 |
| 3_200 | 97.4 | 96.4 | 98.6 | 98.4 | 97.7 | 99.2 |
| 3_201 | 97.8 | 96.7 | 98.9 | 98.8 | 98.5 | 99.2 |
| 250_202 | 93.3 | 93.1 | 93.6 | 97.6 | 96.2 | 99.2 |
| 248_203 | 95.5 | 95.7 | 95.4 | 98.4 | 100.0 | 96.7 |
| 248_10 | 99.3 | 100.0 | 98.6 | 100.0 | 100.0 | 100.0 |
| 1_249 | 98.5 | 99.7 | 97.1 | 99.6 | 99.2 | 100.0 |
| 3_250 | 99.7 | 99.7 | 99.6 | 100.0 | 100.0 | 100.0 |
| 251_18 | 99.0 | 98.3 | 99.6 | 100.0 | 100.0 | 100.0 |
| 6_252 | 97.3 | 99.3 | 95.0 | 99.2 | 99.2 | 99.2 |
| 248_253 | 97.6 | 97.0 | 98.2 | 99.2 | 99.2 | 99.2 |
| 6_254 | 99.0 | 98.7 | 99.3 | 99.6 | 99.2 | 100.0 |
| 3_255 | 98.1 | 97.0 | 99.3 | 98.8 | 97.7 | 100.0 |
| 3_256 | 99.3 | 99.7 | 98.9 | 99.6 | 100.0 | 99.2 |
| 3_257 | 98.5 | 97.7 | 99.3 | 99.2 | 98.5 | 100.0 |
| 3_258 | 97.9 | 97.0 | 98.9 | 98.8 | 97.7 | 100.0 |
| 3_259 | 98.5 | 98.0 | 98.9 | 99.2 | 98.5 | 100.0 |
| 16_260 | 98.1 | 98.3 | 97.9 | 100.0 | 100.0 | 100.0 |
| 10_261 | 99.0 | 99.3 | 98.6 | 98.8 | 98.5 | 99.2 |
| 3_262 | 97.8 | 96.7 | 98.9 | 99.6 | 99.2 | 100.0 |
| 3_263 | 98.1 | 98.3 | 97.9 | 98.4 | 97.7 | 99.2 |
| 3_264 | 97.6 | 96.4 | 98.9 | 98.4 | 97.7 | 99.2 |
| 3_265 | 97.6 | 96.4 | 98.9 | 98.8 | 98.5 | 99.2 |
| 3_266 | 97.8 | 97.0 | 98.6 | 98.4 | 97.7 | 99.2 |
| 248_267 | 95.4 | 95.4 | 95.4 | 98.8 | 100.0 | 97.5 |
| 3_268 | 97.8 | 97.0 | 98.6 | 98.8 | 98.5 | 99.2 |

### Example 2

<Selection of Ovarian Tumor Gene Marker by Comparison with Benign Bone and Soft Tissue Tumor Patients and Benign Breast Disease Patients and Method for Evaluating Ovarian Tumor Discriminant Performance by Single Gene Marker>

In this Example, miRNAs whose expression levels significantly differ between an ovarian tumor and a set of benign bone and soft tissue tumor and breast benign disease were selected as ovarian tumor gene markers. A discriminant(s) was prepared using one or two gene markers in the training cohort, and then, the discrimination accuracy in the validation cohort was calculated. Based on the calculation, the performance of the gene marker(s) to distinguish an ovarian tumor from benign bone and soft tissue tumor and breast benign disease was evaluated.

Specifically, first, miRNA expression levels in the training cohort and the validation cohort obtained in the Reference Example above were added up and normalized in accordance with global normalization.

Then, a gene for diagnosis was selected. Herein, in order to obtain a more highly reliable diagnostic marker(s), only a gene(s) having a gene expression level of 2⁶ or more in 50% or more samples in either one of a group of ovarian tumor patients or a group of benign bone and soft tissue tumor patients and breast benign disease patients was selected. Next, in order to obtain a gene(s) whose gene expression level significantly differs in statistics between the group of ovarian tumor patients and the group of benign bone and soft tissue tumor patients and breast benign disease patients, a two-sided t-test assuming equal variance was carried out, and then, a gene(s) having a P value obtained after the Bonferroni correction of less than 0.01 was selected. Furthermore, in order to select a gene(s) rarely affected by noise at the time of measurement, a gene(s) having an absolute value of the difference (hold change) in gene expression level, which is obtained by logarithmic conversion between the group of ovarian tumor patients and the group of benign bone and soft tissue tumor patients and breast benign disease patients, and which is larger than 0.5, was selected as diagnostic markers for ovarian tumor and for benign bone and soft tissue tumor and breast benign disease. The results are shown in Table 6.

In this way, hsa-miR-4532, hsa-miR-8073, hsa-miR-320b, hsa-miR-1343-3p, hsa-miR-4730, hsa-miR-5100, hsa-miR-4515, hsa-miR-1273g-3p, hsa-miR-1238-5p, hsa-miR-6515-3p, hsa-miR-4525, hsa-miR-614, hsa-miR-1275, hsa-miR-3679-3p, hsa-miR-4718, hsa-miR-4447, hsa-miR-4448, hsa-miR-6766-3p, hsa-miR-3160-5p, hsa-miR-619-5p, hsa-miR-342-5p, hsa-miR-6872-3p, hsa-miR-6798-3p, hsa-miR-665, hsa-miR-6877-5p, hsa-miR-718, hsa-miR-6717-5p, hsa-miR-1199-5p, hsa-miR-6796-3p, hsa-miR-1292-3p, hsa-miR-365a-5p, hsa-miR-150-3p, hsa-miR-3195, hsa-miR-3679-5p, hsa-miR-6076, hsa-miR-6515-5p, hsa-miR-6820-5p, hsa-miR-4634, hsa-miR-187-5p, hsa-miR-6763-5p, hsa-miR-1908-3p, hsa-miR-1181, hsa-miR-6782-5p, hsa-miR-5010-5p, hsa-miR-320a, hsa-miR-296-5p, hsa-miR-1290, hsa-miR-451a, hsa-miR-625-3p, hsa-miR-642a-3p, hsa-miR-483-5p, and hsa-miR-652-5p genes, and the relevant polynucleotides consisting of the nucleotide sequences of SEQ ID NOs: 4, 9, 14, 17, 18, 20, 29, 40, 47, 62, 66, 69, 78, 82, 83, 86, 87, 89, 116, 125, 127, 132, 135, 136, 147, 163, 164, 167, 174, 177, 184, 193, 204 to 215, 248, 256, 260, 264, 265, 268, 269 and 270 were found.

Of them, the genes newly found as a marker for examining the presence or absence of an ovarian tumor are polynucleotides consisting of the nucleotide sequences represented by SEQ ID NOs: 4, 9, 14, 17, 18, 20, 29, 40, 47, 62, 66, 69, 78, 82, 83, 86, 87, 89, 116, 125, 127, 132, 135, 136, 147, 163, 164, 167, 174, 177, 184, 193, and 204 to 215.

A discriminant for determining the presence or absence of an ovarian tumor was further prepared by Fisher's discriminant analysis with the expression levels of these genes in the training cohort as indicators. Specifically, the gene expression level of a polynucleotide consisting of the nucleotide sequence represented by any of SEQ ID NOs: 4, 9, 14, 17, 18, 20, 29, 40, 47, 62, 66, 69, 78, 82, 83, 86, 87, 89, 116, 125, 127, 132, 135, 136, 147, 163, 164, 167, 174, 177, 184, 193, 204 to 215, 248, 256, 260, 264, 265, 268, 269 and 270 found above was input to Formula 2 to prepare a discriminant. Calculated accuracy, sensitivity, and specificity in the training cohort are shown in Table 7. In this respect, discriminant coefficients and constant terms are shown in Table 8.

Next, accuracy, sensitivity, and specificity in the validation cohort were calculated using the discriminant prepared above and the discriminant performance of the selected polynucleotides was validated using independent samples (Table 7). For example, the rate of detecting an ovarian tumor was calculated using the threshold value (11.59) of the gene expression level of the nucleotide sequence represented by SEQ ID NO: 20, for use in discriminating both groups set in the training cohort. As a result, 122 true positives, 110 true negatives, 1 false positive and 9 false negatives were obtained in the validation cohort. From these values, 95.9% accuracy, 93.1% sensitivity, and 99.1% specificity were obtained as the detection performance. Similarly, the sensitivities of all polynucleotides represented by SEQ ID NOs: 4, 9, 14, 17, 18, 20, 29, 40, 47, 62, 66, 69, 78, 82, 83, 86, 87, 89, 116, 125, 127, 132, 135, 136, 147, 163, 164, 167, 174, 177, 184, 193, 204 to 215, 248, 256, 260, 264, 265, 268, 269 and 270 were 69.5%, 76.3%, 89.3%, 73.3%, 71.8%, 93.1%, 70.2%, 73.3%, 78.6%, 81.7%, 62.6%, 71.0%, 75.6%, 78.6%, 74.8%, 81.7%, 68.7%, 82.4%, 72.5%, 81.7%, 64.9%, 81.7%, 84.0%, 80.2%, 77.1%, 87.0%, 64.1%, 80.9%, 89.3%, 87.8%, 68.7%, 77.1%, 84.7%, 66.4%, 75.6%, 61.1%, 68.7%, 73.3%, 79.4%, 61.8%, 81.7%, 84.0%, 60.3%, 80.9%, 80.9%, 87.8%, 96.2%, 69.5%, 76.3%, 71.8%, 66.4% and 79.4%, respectively; and the specificities thereof were 61.3%, 67.6%, 53.2%, 53.2%, 45.9%, 99.1%, 45.0%, 61.3%, 62.2%, 57.7%, 64.0%, 55.0%, 65.8%, 49.5%, 28.8%, 40.5%, 36.0%, 48.6%, 37.8%, 49.5%, 56.8%, 40.5%, 39.6%, 62.2%, 65.8%, 43.2%, 68.5%, 43.2%, 28.8%, 51.4%, 65.8%, 40.5%, 61.3%, 73.0%, 49.5%, 74.8%, 73.0%, 55.0%, 48.6%, 59.5%, 44.1%, 37.8%, 71.2%, 32.4%, 50.5%, 76.6%, 89.2%, 49.5%, 47.7%, 56.8%, 72.1% and 41.4%, respectively (Table 7).

Also, a discriminant was constructed using each of all polynucleotides represented by SEQ ID NOs: 4, 9, 14, 17, 18, 20, 29, 40, 47, 62, 66, 69, 78, 82, 83, 86, 87, 89, 116, 125, 127, 132, 135, 136, 147, 163, 164, 167, 174, 177, 184, 193, 204 to 215, 248, 256, 260, 264, 265, 268, 269 and 270 in combination with another polynucleotide appropriately selected; in other words, discriminants each were constructed using two polynucleotides. The discrimination accuracy of an ovarian tumor in the validation cohort by the discriminants thus constructed increased compared with the discriminants using individual genes alone. Specifically, discriminants prepared using combinations of SEQ ID NOs: 256 and 4,260 and 9,256 and 14, 20 and 17, 20 and 18, 20 and 69, 20 and 29, 256 and 40, 260 and 47, 20 and 62, 20 and 66, 20 and 69, 20 and 78, 260 and 82, 20 and 83, 207 and 86, 20 and 87, 20 and 89, 20 and 116, 260 and 125, 20 and 127, 20 and 132, 260 and 135, 20 and 136, 147 and 164, 256 and 163, 20 and 164, 260 and 167, 20 and 174, 20 and 177, 260 and 184, 260 and 193, 260 and 204, 260 and 205, 260 and 206, 20 and 207, 260 and 208, 20 and 209, 256 and 210, 260 and 211, 256 and 212, 20 and 213, 20 and 214, 20 and 215, 269 and 248, 260 and 256, 260 and 69, 20 and 264, 20 and 265, 260 and 268, 20 and 269, and 260 and 270 had discrimination accuracies, in the validation cohort, of 83.5%, 93.8%, 86.8%, 97.5%, 96.3%, 99.6%, 96.7%, 83.9%, 93.4%, 96.3%, 97.9%, 99.6%, 97.5%, 94.2%, 97.5%, 77.3%, 97.9%, 96.3%, 96.7%, 93.4%, 98.8%, 96.3%, 93.8%, 97.1%, 90.9%, 83.1%, 97.5%, 93.4%, 96.3%, 96.3%, 96.7%, 93.4%, 93.8%, 93.4%, 93.4%, 98.3%, 95.9%, 96.3%, 84.3%, 95.5%, 85.1%, 96.3%, 96.3%, 96.3%, 87.2%, 97.5%, 98.3%, 96.7%, 96.3%, 93.4%, 96.3% and 93.8%, respectively (Table 9).

From the above, all polynucleotides consisting of the nucleotide sequences represented by SEQ ID NOs: 4, 9, 14, 17, 18, 20, 29, 40, 47, 62, 66, 69, 78, 82, 83, 86, 87, 89, 116, 125, 127, 132, 135, 136, 147, 163, 164, 167, 174, 177, 184, 193, 204 to 215, 248, 256, 260, 264, 265, 268, 269 and 270 are genes or nucleic acids capable of discriminating an ovarian tumor patient from benign bone and soft tissue tumor patients and benign breast disease patients with high accuracy if these are used singly or in combinations of two or more.

TABLE 6

| | | | Fold change |
|---|---|---|---|
| | | | of benign bone |
| | | | and soft tissue |
| | | | tumor patient |
| | | | and benign |
| | | P value after | breast disease |
| SEQ | | Bonferroni | patient to ovarian |
| ID NO. | Name of gene | correction | tumor patient |
| 4 | hsa-miR-4532 | 1.94.E−22 | −0.67 |
| 9 | hsa-miR-8073 | 5.36.E−53 | −0.97 |
| 14 | hsa-miR-320b | 2.63.E−37 | −1.11 |
| 17 | hsa-miR-1343-3p | 2.00.E−20 | −0.53 |
| 18 | hsa-miR-4730 | 1.19.E−11 | −0.60 |
| 20 | hsa-miR-5100 | 4.36.E−235 | −3.07 |
| 29 | hsa-miR-4515 | 8.99.E−13 | −0.69 |
| 40 | hsa-miR-1273g-3p | 1.55.E−30 | −0.91 |
| 47 | hsa-miR-1238-5p | 4.28.E−35 | −1.03 |
| 62 | hsa-miR-6515-3p | 3.65.E−40 | −0.52 |
| 66 | hsa-miR-4525 | 3.74.E−26 | −1.08 |
| 69 | hsa-miR-614 | 2.07.E−10 | 0.52 |
| 78 | hsa-miR-1275 | 7.46.E−34 | −0.59 |
| 82 | hsa-miR-3679-3p | 1.01.E−21 | −0.51 |
| 83 | hsa-miR-4718 | 3.14.E−03 | −0.55 |
| 86 | hsa-miR-4447 | 2.33.E−14 | −0.55 |
| 87 | hsa-miR-4448 | 2.82.E−06 | −0.64 |
| 89 | hsa-miR-6766-3p | 4.01.E−29 | −0.71 |
| 116 | hsa-miR-3160-5p | 4.57.E−09 | −0.84 |
| 125 | hsa-miR-619-5p | 2.81.E−15 | −0.97 |
| 127 | hsa-miR-342-5p | 8.24.E−09 | 0.63 |
| 132 | hsa-miR-6872-3p | 3.39.E−10 | −0.57 |
| 135 | hsa-miR-6798-3p | 7.08.E−28 | −0.88 |
| 136 | hsa-miR-665 | 1.06.E−38 | −0.91 |
| 147 | hsa-miR-6877-5p | 1.32.E−40 | −0.69 |
| 163 | hsa-miR-718 | 1.44.E−14 | −0.55 |
| 164 | hsa-miR-6717-5p | 1.85.E−22 | 1.07 |
| 167 | hsa-miR-1199-5p | 3.54.E−16 | −0.72 |
| 174 | hsa-miR-6796-3p | 7.02.E−13 | −0.53 |
| 177 | hsa-miR-1292-3p | 4.01.E−28 | −0.90 |
| 184 | hsa-miR-365a-5p | 4.94.E−24 | 0.54 |
| 193 | hsa-miR-150-3p | 1.94.E−08 | −0.53 |
| 204 | hsa-miR-3195 | 1.78.E−43 | −0.70 |
| 205 | hsa-miR-3679-5p | 3.16.E−37 | 0.65 |
| 206 | hsa-miR-6076 | 1.67.E−31 | −0.73 |
| 207 | hsa-miR-6515-5p | 2.68.E−28 | 1.21 |
| 208 | hsa-miR-6820-5p | 1.67.E−23 | 0.50 |
| 209 | hsa-miR-4634 | 1.10.E−22 | −0.52 |
| 210 | hsa-miR-187-5p | 1.01.E−17 | −0.55 |
| 211 | hsa-miR-6763-5p | 1.15.E−12 | 0.55 |
| 212 | hsa-miR-1908-3p | 1.07.E−11 | −0.58 |
| 213 | hsa-miR-1181 | 1.31.E−10 | −0.52 |
| 214 | hsa-miR-6782-5p | 1.68.E−09 | 0.53 |
| 215 | hsa-miR-5010-5p | 3.83.E−06 | 0.59 |
| 248 | hsa-miR-320a | 1.60.E−23 | −0.65 |
| 256 | hsa-miR-296-5p | 1.90.E−87 | −0.68 |
| 260 | hsa-miR-1290 | 7.49.E−208 | −4.86 |
| 264 | hsa-miR-451a | 1.06.E−21 | −1.55 |
| 265 | hsa-miR-625-3p | 6.47.E−20 | −0.80 |
| 268 | hsa-miR-642a-3p | 7.46.E−21 | −0.66 |
| 269 | hsa-miR-483-5p | 9.83.E−30 | 1.00 |
| 270 | hsa-miR-652-5p | 1.89.E−12 | −0.69 |

TABLE 7

| | Training cohort | | | Validation cohort | | |
|---|---|---|---|---|---|---|
| | Accuracy | Sensitivity | Specificity | Accuracy | Sensitivity | |
| SEQ ID NO. | (%) | (%) | (%) | (%)) | (%) | Specificity (%) |
| 4 | 66.8 | 78.5 | 52.9 | 65.7 | 69.5 | 61.3 |
| 9 | 73.4 | 78.2 | 67.7 | 72.3 | 76.3 | 67.6 |
| 14 | 69.6 | 82.2 | 54.9 | 72.7 | 89.3 | 53.2 |
| 17 | 63.6 | 71.9 | 53.7 | 64.0 | 73.3 | 53.2 |
| 18 | 66.4 | 78.9 | 51.8 | 59.9 | 71.8 | 45.9 |
| 20 | 95.7 | 93.1 | 98.8 | 95.9 | 93.1 | 99.1 |
| 29 | 58.6 | 70.3 | 44.7 | 58.7 | 70.2 | 45.0 |
| 40 | 69.6 | 76.6 | 61.5 | 67.8 | 73.3 | 61.3 |
| 47 | 68.0 | 75.9 | 58.8 | 71.1 | 78.6 | 62.2 |
| 62 | 73.4 | 80.9 | 64.6 | 70.7 | 81.7 | 57.7 |
| 66 | 64.8 | 66.3 | 63.0 | 63.2 | 62.6 | 64.0 |
| 69 | 59.6 | 71.0 | 46.3 | 63.6 | 71.0 | 55.0 |
| 78 | 70.4 | 73.9 | 66.1 | 71.1 | 75.6 | 65.8 |
| 82 | 67.0 | 80.5 | 51.0 | 65.3 | 78.6 | 49.5 |
| 83 | 56.6 | 78.9 | 30.4 | 53.7 | 74.8 | 28.8 |
| 86 | 61.6 | 81.8 | 37.7 | 62.8 | 81.7 | 40.5 |
| 87 | 57.0 | 71.6 | 39.7 | 53.7 | 68.7 | 36.0 |
| 89 | 70.4 | 83.5 | 54.9 | 66.9 | 82.4 | 48.6 |
| 116 | 55.5 | 70.3 | 38.1 | 56.6 | 72.5 | 37.8 |
| 125 | 64.3 | 78.2 | 47.9 | 66.9 | 81.7 | 49.5 |
| 127 | 59.3 | 65.0 | 52.5 | 61.2 | 64.9 | 56.8 |
| 132 | 61.1 | 82.2 | 36.2 | 62.8 | 81.7 | 40.5 |
| 135 | 73.4 | 88.4 | 55.6 | 63.6 | 84.0 | 39.6 |
| 136 | 74.8 | 84.8 | 63.0 | 71.9 | 80.2 | 62.2 |
| 147 | 71.2 | 73.9 | 68.1 | 71.9 | 77.1 | 65.8 |
| 163 | 70.9 | 89.4 | 49.0 | 66.9 | 87.0 | 43.2 |
| 164 | 63.4 | 62.7 | 64.2 | 66.1 | 64.1 | 68.5 |
| 167 | 63.4 | 78.9 | 45.1 | 63.6 | 80.9 | 43.2 |
| 174 | 63.4 | 85.8 | 37.0 | 61.6 | 89.3 | 28.8 |
| 177 | 68.0 | 85.1 | 47.9 | 71.1 | 87.8 | 51.4 |
| 184 | 65.2 | 67.7 | 62.3 | 67.4 | 68.7 | 65.8 |
| 193 | 60.5 | 82.2 | 35.0 | 60.3 | 77.1 | 40.5 |
| 204 | 77.1 | 88.4 | 63.8 | 74.0 | 84.7 | 61.3 |
| 205 | 72.0 | 73.9 | 69.6 | 69.4 | 66.4 | 73.0 |
| 206 | 69.8 | 77.6 | 60.7 | 63.6 | 75.6 | 49.5 |
| 207 | 68.4 | 66.0 | 71.2 | 67.4 | 61.1 | 74.8 |
| 208 | 68.8 | 70.0 | 67.3 | 70.7 | 68.7 | 73.0 |
| 209 | 70.9 | 79.2 | 61.1 | 64.9 | 73.3 | 55.0 |
| 210 | 69.8 | 82.2 | 55.3 | 65.3 | 79.4 | 48.6 |
| 211 | 60.2 | 67.7 | 51.4 | 60.7 | 61.8 | 59.5 |
| 212 | 64.1 | 82.8 | 42.0 | 64.5 | 81.7 | 44.1 |
| 213 | 62.5 | 84.5 | 36.6 | 62.8 | 84.0 | 37.8 |
| 214 | 66.1 | 67.7 | 64.2 | 65.3 | 60.3 | 71.2 |
| 215 | 57.7 | 81.2 | 30.0 | 58.7 | 80.9 | 32.4 |
| 248 | 64.3 | 75.6 | 51.0 | 66.9 | 80.9 | 50.5 |
| 256 | 79.3 | 83.8 | 73.9 | 82.6 | 87.8 | 76.6 |
| 260 | 93.6 | 94.7 | 92.2 | 93.0 | 96.2 | 89.2 |
| 264 | 65.5 | 77.2 | 51.8 | 60.3 | 69.5 | 49.5 |
| 265 | 67.3 | 82.5 | 49.4 | 63.2 | 76.3 | 47.7 |
| 268 | 64.1 | 69.0 | 58.4 | 64.9 | 71.8 | 56.8 |
| 269 | 69.6 | 70.0 | 69.3 | 69.0 | 66.4 | 72.1 |
| 270 | 61.6 | 78.5 | 41.6 | 62.0 | 79.4 | 41.4 |

TABLE 8

| SEQ | | Constant |
|---|---|---|
| ID NO. | Coefficient | term |
| 4 | 1.163 | 15.873 |
| 9 | 1.284 | 9.651 |
| 14 | 0.874 | 5.236 |
| 17 | 1.441 | 10.642 |
| 18 | 0.889 | 6.725 |
| 20 | 1.095 | 12.683 |
| 29 | 0.867 | 5.603 |
| 40 | 0.985 | 8.121 |
| 47 | 0.939 | 6.768 |
| 62 | 1.991 | 14.542 |
| 66 | 0.800 | 8.178 |
| 69 | 1.039 | 10.209 |
| 78 | 1.705 | 13.533 |
| 82 | 1.520 | 10.204 |
| 83 | 0.601 | 4.544 |
| 86 | 1.088 | 7.271 |
| 87 | 0.642 | 5.196 |
| 89 | 1.250 | 7.650 |
| 116 | 0.585 | 3.824 |
| 125 | 0.635 | 4.619 |
| 127 | 0.756 | 4.670 |
| 132 | 0.889 | 4.985 |
| 135 | 0.956 | 5.435 |
| 136 | 1.078 | 7.870 |
| 147 | 1.515 | 11.241 |
| 163 | 1.101 | 7.632 |
| 164 | 0.695 | 4.807 |
| 167 | 0.894 | 4.845 |
| 174 | 1.038 | 6.049 |
| 177 | 0.942 | 5.418 |
| 184 | 1.423 | 10.156 |
| 193 | 0.891 | 5.086 |
| 204 | 1.598 | 13.356 |
| 205 | 1.574 | 12.205 |
| 206 | 1.283 | 9.375 |
| 207 | 0.712 | 3.814 |
| 208 | 1.578 | 11.937 |
| 209 | 1.474 | 13.007 |
| 210 | 1.184 | 9.382 |
| 211 | 1.069 | 7.298 |
| 212 | 0.963 | 6.212 |
| 213 | 0.992 | 5.528 |
| 214 | 0.956 | 5.581 |
| 215 | 0.681 | 3.993 |
| 248 | 1.158 | 7.969 |
| 256 | 2.371 | 18.848 |
| 260 | 0.629 | 4.171 |
| 264 | 0.481 | 2.967 |
| 265 | 0.895 | 5.115 |
| 268 | 1.077 | 8.405 |
| 269 | 0.861 | 5.053 |
| 270 | 0.831 | 4.838 |

TABLE 9

| | Training cohort | | | Validation cohort | | |
|---|---|---|---|---|---|---|
| | Accuracy | Sensitivity | Specificity | Accuracy | Sensitivity | |
| SEQ ID NO. | (%) | (%) | (%) | (%) | (%) | Specificity (%) |
| 256_4 | 84.1 | 90.1 | 77.0 | 83.5 | 83.2 | 83.8 |
| 260_9 | 93.4 | 95.0 | 91.4 | 93.8 | 96.2 | 91.0 |
| 256_14 | 82.3 | 87.1 | 76.7 | 86.8 | 92.4 | 80.2 |
| 20_17 | 96.1 | 94.4 | 98.1 | 97.5 | 96.9 | 98.2 |
| 20_18 | 95.4 | 92.7 | 98.4 | 96.3 | 93.9 | 99.1 |
| 20_69 | 98.6 | 98.3 | 98.8 | 99.6 | 100.0 | 99.1 |
| 20_29 | 96.4 | 94.4 | 98.8 | 96.7 | 95.4 | 98.2 |
| 256_40 | 80.4 | 85.1 | 74.7 | 83.9 | 87.0 | 80.2 |
| 260_47 | 93.4 | 94.7 | 91.8 | 93.4 | 96.2 | 90.1 |
| 20_62 | 96.4 | 94.4 | 98.8 | 96.3 | 93.9 | 99.1 |
| 20_66 | 96.4 | 94.4 | 98.8 | 97.9 | 96.9 | 99.1 |
| 20_69 | 98.6 | 98.3 | 98.8 | 99.6 | 100.0 | 99.1 |
| 20_78 | 96.4 | 94.4 | 98.8 | 97.5 | 95.4 | 100.0 |
| 260_82 | 93.4 | 95.4 | 91.1 | 94.2 | 96.2 | 91.9 |
| 20_83 | 96.8 | 95.4 | 98.4 | 97.5 | 96.9 | 98.2 |
| 207_86 | 79.1 | 80.9 | 77.0 | 77.3 | 74.0 | 81.1 |
| 20_87 | 96.2 | 93.7 | 99.2 | 97.9 | 96.9 | 99.1 |
| 20_89 | 95.7 | 93.1 | 98.8 | 96.3 | 93.9 | 99.1 |
| 20_116 | 96.2 | 94.1 | 98.8 | 96.7 | 95.4 | 98.2 |
| 260_125 | 93.4 | 94.4 | 92.2 | 93.4 | 96.2 | 90.1 |
| 20_127 | 98.0 | 97.0 | 99.2 | 98.8 | 98.5 | 99.1 |
| 20_132 | 95.9 | 93.1 | 99.2 | 96.3 | 93.9 | 99.1 |
| 260_135 | 94.5 | 95.0 | 93.8 | 93.8 | 95.4 | 91.9 |
| 20_136 | 96.1 | 94.1 | 98.4 | 97.1 | 95.4 | 99.1 |
| 147_164 | 88.4 | 88.8 | 87.9 | 90.9 | 91.6 | 90.1 |
| 256_163 | 80.2 | 85.8 | 73.5 | 83.1 | 88.5 | 76.6 |
| 20_164 | 97.1 | 95.4 | 99.2 | 97.5 | 96.2 | 99.1 |
| 260_167 | 93.4 | 95.0 | 91.4 | 93.4 | 95.4 | 91.0 |
| 20_174 | 95.9 | 93.1 | 99.2 | 96.3 | 93.9 | 99.1 |
| 20_177 | 95.7 | 93.1 | 98.8 | 96.3 | 93.9 | 99.1 |
| 260_184 | 95.7 | 97.0 | 94.2 | 96.7 | 96.9 | 96.4 |
| 260_193 | 93.2 | 94.7 | 91.4 | 93.4 | 96.2 | 90.1 |
| 260_204 | 93.4 | 95.0 | 91.4 | 93.8 | 96.2 | 91.0 |
| 260_205 | 94.5 | 95.4 | 93.4 | 93.4 | 94.7 | 91.9 |
| 260_206 | 93.9 | 95.0 | 92.6 | 93.4 | 96.2 | 90.1 |
| 20_207 | 96.8 | 94.7 | 99.2 | 98.3 | 98.5 | 98.2 |
| 260_208 | 96.1 | 97.0 | 94.9 | 95.9 | 95.4 | 96.4 |
| 20_209 | 95.5 | 92.7 | 98.8 | 96.3 | 93.9 | 99.1 |
| 256_210 | 81.2 | 86.8 | 74.7 | 84.3 | 88.5 | 79.3 |
| 260_211 | 95.0 | 96.0 | 93.8 | 95.5 | 96.2 | 94.6 |
| 256_212 | 82.3 | 87.5 | 76.3 | 85.1 | 90.1 | 79.3 |
| 20_213 | 95.5 | 92.7 | 98.8 | 96.3 | 93.9 | 99.1 |
| 20_214 | 95.7 | 93.1 | 98.8 | 96.3 | 94.7 | 98.2 |
| 20_215 | 95.5 | 92.7 | 98.8 | 96.3 | 93.9 | 99.1 |
| 269_248 | 78.2 | 79.2 | 77.0 | 87.2 | 86.3 | 88.3 |
| 260_256 | 95.9 | 97.0 | 94.6 | 97.5 | 97.7 | 97.3 |
| 260_69 | 97.7 | 98.3 | 96.9 | 98.3 | 98.5 | 98.2 |
| 20_264 | 95.9 | 93.4 | 98.8 | 96.7 | 94.7 | 99.1 |
| 20_265 | 95.5 | 92.7 | 98.8 | 96.3 | 93.9 | 99.1 |
| 260_268 | 93.8 | 95.0 | 92.2 | 93.4 | 96.2 | 90.1 |
| 20_269 | 95.5 | 92.7 | 98.8 | 96.3 | 93.9 | 99.1 |
| 260_270 | 93.4 | 95.7 | 90.7 | 93.8 | 95.4 | 91.9 |

### Example 3

<Selection of Ovarian Tumor Gene Marker by Comparison with Patients Having a Cancer Other than Ovarian Cancer and Method for Evaluating Ovarian Tumor Discriminant Performance by Single Gene Marker>

In this Example, miRNAs whose expression level significantly differs between an ovarian tumor and a cancer other than ovarian cancer, such as breast cancer, biliary cancer, pancreatic cancer, colorectal cancer, esophagus cancer, stomach cancer, liver cancer and lung cancer, were selected as ovarian tumor gene markers. A discriminant(s) was prepared using one or two gene markers in the training cohort, and then, the discrimination accuracy in the validation cohort was calculated. Based on the calculation, the performance of the gene marker(s) to distinguish an ovarian tumor from a cancer other than ovarian cancer was evaluated.

Specifically, first, miRNA expression levels in the training cohort and the validation cohort obtained in the Reference Example above were added up and normalized in accordance with global normalization.

Then, genes for diagnosis were selected. Herein, in order to obtain a more highly reliable diagnostic marker, only a gene having a gene expression level of 2⁶ or more in 50% or more samples in either one of a group of ovarian tumor patients or a group of patients having a cancer other than ovarian cancer was selected. Next, in order to obtain a gene(s) whose gene expression level significantly differs in statistics between the ovarian tumor patient group or the group of patients having a cancer other than ovarian cancer, a two-sided t-test assuming equal variance was carried out and a gene(s) having a P value obtained after the Bonferroni correction of less than 0.01 was selected. Furthermore, in order to select a gene(s) rarely affected by noise at the time of measurement, a gene(s) having an absolute value of the difference (hold change) in gene expression level, which is obtained by logarithmic conversion, between the group of ovarian tumor patients and the group of patients having a cancer other than ovarian cancer, and which is larger than 0.5, was selected as a diagnostic marker(s) for an ovarian tumor and a cancer other than ovarian cancer. The results are shown in Table 10.

In this way, hsa-miR-4532, hsa-miR-1233-5p, hsa-miR-1343-3p, hsa-miR-6787-5p, hsa-miR-614, hsa-miR-939-5p, hsa-miR-1275, hsa-miR-3679-3p, hsa-miR-8059, hsa-miR-6766-3p, hsa-miR-6131, hsa-miR-3194-3p, hsa-miR-2467-3p, hsa-miR-8063, hsa-miR-342-5p, hsa-miR-4433a-5p, hsa-miR-6798-3p, hsa-miR-7150, hsa-miR-6746-5p, hsa-miR-6717-5p, hsa-miR-6769a-5p, hsa-miR-3131, hsa-miR-6510-5p, hsa-miR-6515-5p, hsa-miR-6763-5p, hsa-miR-6870-5p, hsa-miR-6124, hsa-miR-1249-5p, hsa-miR-6511b-5p, hsa-miR-1254, hsa-miR-4727-3p, hsa-miR-4259, hsa-miR-4771, hsa-miR-3622a-5p, hsa-miR-4480, hsa-miR-4740-5p, hsa-miR-6777-5p, hsa-miR-451a, hsa-miR-92a-3p, hsa-miR-422a, hsa-miR-642a-3p, hsa-miR-24-3p, hsa-miR-23b-3p, hsa-miR-23a-3p, hsa-miR-92b-3p and hsa-miR-22-3p genes, and the relevant polynucleotides consisting of the nucleotide sequences of SEQ ID NOs: 4, 11, 17, 68, 69, 76, 78, 82, 85, 89, 99, 103, 105, 109, 127, 128, 135, 145, 160, 164, 169, 196, 198, 207, 211, 216 to 227,264,266, 267, 268, 271, 272, 273, 274 and 275 were found.

Of them, the genes newly found as the marker for examining the presence or absence of an ovarian tumor are polynucleotides consisting of the nucleotide sequences represented by SEQ ID NOs: 4, 11, 17, 68, 69, 76, 78, 82, 85, 89, 99, 103, 105, 109, 127, 128, 135, 145, 160, 164, 169, 196, 198, 207, 211 and 216 to 227.

A discriminant for determining the presence or absence of an ovarian tumor was further prepared by Fisher's discriminant analysis with the expression levels of these genes in the training cohort as indicators. Specifically, the gene expression level of a polynucleotide consisting of the nucleotide sequence represented by any of SEQ ID NOs: 4, 11, 17, 68, 69, 76, 78, 82, 85, 89, 99, 103, 105, 109, 127, 128, 135, 145, 160, 164, 169, 196, 198, 207, 211, 216 to 227, 264, 266, 267, 268, 271, 272, 273, 274 and 275 found above was input to Formula 2 to prepare a discriminant. Calculated accuracy, sensitivity, and specificity in the training cohort are shown in Table 11. In this respect, discriminant coefficients and constant terms are shown in Table 12.

Next, accuracy, sensitivity, and specificity in the validation cohort were calculated using the discriminant prepared above and the discriminant performance of the selected polynucleotides was validated using independent samples (Table 11). For example, the rate of detecting an ovarian tumor was calculated using the threshold value (8.07) of the gene expression level of the nucleotide sequence represented by SEQ ID NO: 17, for use in discriminating both groups set in the training cohort. As a result, 97 true positives, 74 true negatives, 46 false positives and 34 false negatives were obtained in the validation cohort. From these values, 68.1% accuracy, 74.0% sensitivity, and 61.7% specificity were obtained as the detection performance. Similarly, the sensitivities of all polynucleotides represented by SEQ ID NOs: 4, 11, 17, 68, 69, 76, 78, 82, 85, 89, 99, 103, 105, 109, 127, 128, 135, 145, 160, 164, 169, 196, 198, 207, 211, and 216 to 227 were 65.6%, 62.6%, 74.0%, 74.8%, 67.9%, 63.4%, 74.0%, 77.1%, 67.9%, 80.2%, 61.1%, 65.6%, 68.7%, 64.9%, 60.3%, 80.9%, 80.2%, 74.8%, 54.2%, 64.1%, 63.4%, 58.8%, 77.1%, 57.3%, 61.1%, 80.9%, 78.6%, 71.8%, 58.8%, 59.5%, 71.0%, 54.2%, 66.4%, 55.7%, 65.6%, 66.4%, 48.9%, 70.2%, 62.6%, 77.1%, 63.4%, 62.6%, 65.6%, 67.2%, 61.1% and 63.4%, respectively, and the specificities thereof were 51.7%, 59.2%, 61.7%, 60.0%, 71.7%, 66.7%, 60.0%, 58.3%, 66.7%, 47.5%, 71.7%, 57.5%, 48.3%, 50.0%, 72.5%, 40.0%, 46.7%, 65.0%, 66.7%, 82.5%, 70.0%, 61.7%, 45.0%, 81.7%, 66.7%, 49.2%, 56.7%, 50.0%, 65.8%, 67.5%, 62.5%, 65.8%, 61.7%, 61.7%, 60.8%, 55.0%, 60.0%, 65.0%, 67.5%, 39.2%, 53.3%, 68.3%, 64.2%, 63.3%, 64.2% and 63.3%, respectively (Table 11).

Also, a discriminant was constructed using each of all polynucleotides represented by SEQ ID NOs: 4, 11, 17, 68, 69, 76, 78, 82, 85, 89, 99, 103, 105, 109, 127, 128, 135, 145, 160, 164, 169, 196, 198, 207, 211, 216 to 227,264,266, 267, 268, 271, 272, 273, 274 and 275 in combination with another polynucleotide appropriately selected; in other words, discriminants each were constructed using two polynucleotides. The discrimination accuracy of an ovarian tumor in the validation cohort by the discriminants thus constructed increased, compared with the discriminants using individual genes alone. Specifically, discriminants prepared using combinations of SEQ ID NOs: 164 and 4, 17 and 11, 17 and 82, 82 and 68, 69 and 82, 82 and 76, 164 and 78, 164 and 82, 82 and 85, 164 and 89, 145 and 99, 217 and 103, 164 and 105, 69 and 109, 82 and 127, 164 and 128, 17 and 135, 145 and 164, 82 and 160, 164 and 82, 82 and 169, 82 and 196, 164 and 198, 82 and 207, 82 and 211, 164 and 216, 164 and 217, 164 and 218, 82 and 219, 82 and 220, 82 and 221, 145 and 222, 82 and 223, 78 and 224, 82 and 225, 217 and 226, 82 and 227, 82 and 264, 145 and 266, 145 and 267, 160 and 268, 82 and 271, 82 and 272, 164 and 273, 145 and 274, and 217 and 275 had discrimination accuracies, in the validation cohort, of 73.3%, 74.1%, 80.5%, 76.1%, 79.7%, 80.1%, 78.5%, 83.3%, 79.7%, 82.5%, 73.7%, 72.9%, 74.5%, 71.3%, 79.3%, 77.7%, 77.3%, 78.5%, 82.5%, 83.3%, 80.5%, 83.3%, 77.7%, 78.5%, 76.5%, 77.7%, 78.5%, 76.9%, 76.9%, 82.5%, 80.1%, 73.3%, 75.3%, 74.1%, 72.5%, 71.7%, 74.1%, 74.1%, 73.3%, 73.7%, 80.9%, 75.3%, 71.7%, 73.3%, 73.3% and 73.7%, respectively (Table 13).

From the above, all polynucleotides consisting of the nucleotide sequences represented by SEQ ID NOs: 4, 11, 17, 68, 69, 76, 78, 82, 85, 89, 99, 103, 105, 109, 127, 128, 135, 145, 160, 164, 169, 196, 198, 207, 211, 216 to 227, 264, 266, 267, 268, 271, 272, 273, 274 and 275 are genes or nucleic acids capable of discriminating an ovarian tumor patient from patients having a cancer other than ovarian cancer with high accuracy if these are used singly or in combinations of two or more.

TABLE 10

| | | | Fold change |
|---|---|---|---|
| | | | of patient |
| | | | having cancer |
| | | | other than |
| | | P value after | ovarian cancer |
| SEQ | | Bonferroni | to ovarian |
| ID NO. | Name of gene | correction | tumor patient |
| 4 | hsa-miR-4532 | 7.46.E−09 | −0.51 |
| 11 | hsa-miR-1233-5p | 5.68.E−14 | −0.53 |
| 17 | hsa-miR-1343-3p | 1.00.E−30 | 0.90 |
| 68 | hsa-miR-6787-5p | 1.25.E−24 | 0.58 |
| 69 | hsa-miR-614 | 5.61.E−27 | 0.84 |
| 76 | hsa-miR-939-5p | 5.01.E−13 | 0.57 |
| 78 | hsa-miR-1275 | 1.64.E−27 | −0.53 |
| 82 | hsa-miR-3679-3p | 6.03.E−25 | −0.57 |
| 85 | hsa-miR-8059 | 1.86.E−11 | 0.56 |
| 89 | hsa-miR-6766-3p | 1.12.E−22 | −0.65 |
| 99 | hsa-miR-6131 | 5.83.E−09 | 1.24 |
| 103 | hsa-miR-3194-3p | 2.11.E−07 | 0.84 |
| 105 | hsa-miR-2467-3p | 9.35.E−04 | 0.58 |
| 109 | hsa-miR-8063 | 2.24.E−08 | −0.50 |
| 127 | hsa-miR-342-5p | 5.64.E−12 | 0.75 |
| 128 | hsa-miR-4433a-5p | 2.92.E−11 | −0.50 |
| 135 | hsa-miR-6798-3p | 5.26.E−19 | −0.72 |
| 145 | hsa-miR-7150 | 3.07.E−30 | −0.54 |
| 160 | hsa-miR-6746-5p | 7.58.E−15 | 0.63 |
| 164 | hsa-miR-6717-5p | 6.16.E−29 | 1.38 |
| 169 | hsa-miR-6769a-5p | 2.10.E−14 | 0.61 |
| 196 | hsa-miR-3131 | 1.75.E−11 | 0.51 |
| 198 | hsa-miR-6510-5p | 8.28.E−17 | −0.71 |
| 207 | hsa-miR-6515-5p | 4.94.E−24 | 1.18 |
| 211 | hsa-miR-6763-5p | 8.39.E−11 | 0.58 |
| 216 | hsa-miR-6870-5p | 1.33.E−27 | −0.78 |
| 217 | hsa-miR-6124 | 1.25.E−26 | −0.75 |
| 218 | hsa-miR-1249-5p | 1.73.E−21 | −0.78 |
| 219 | hsa-miR-6511b-5p | 1.65.E−15 | 0.87 |
| 220 | hsa-miR-1254 | 8.73.E−14 | 0.69 |
| 221 | hsa-miR-4727-3p | 1.89.E−09 | 0.71 |
| 222 | hsa-miR-4259 | 1.87.E−08 | 0.71 |
| 223 | hsa-miR-4771 | 5.92.E−08 | 0.76 |
| 224 | hsa-miR-3622a-5p | 1.63.E−07 | 0.52 |
| 225 | hsa-miR-4480 | 1.26.E−06 | 0.73 |
| 226 | hsa-miR-4740-5p | 7.43.E−05 | 0.60 |
| 227 | hsa-miR-6777-5p | 1.46.E−04 | 0.50 |
| 264 | hsa-miR-451a | 2.61.E−08 | 0.99 |
| 266 | hsa-miR-92a-3p | 8.20.E−10 | 0.77 |
| 267 | hsa-miR-422a | 2.83.E−08 | −0.67 |
| 268 | hsa-miR-642a-3p | 5.51.E−13 | −0.53 |
| 271 | hsa-miR-24-3p | 5.29.E−14 | 0.91 |
| 272 | hsa-miR-23b-3p | 9.72.E−12 | 1.11 |
| 273 | hsa-miR-23a-3p | 9.52.E−10 | 1.09 |
| 274 | hsa-miR-92b-3p | 2.81.E−09 | 0.73 |
| 275 | hsa-miR-22-3p | 9.10.E−09 | 0.86 |

TABLE 11

| | Training cohort | | | Validation cohort | | |
|---|---|---|---|---|---|---|
| | Accuracy | Sensitivity | Specificity | Accuracy | Sensitivity | |
| SEQ ID NO. | (%) | (%) | (%) | (%) | (%) | Specificity (%) |
| 17 | 65.7 | 77.9 | 52.5 | 68.1 | 74.0 | 61.7 |
| 145 | 65.5 | 70.6 | 60.0 | 70.1 | 74.8 | 65.0 |
| 164 | 67.6 | 61.4 | 74.3 | 72.9 | 64.1 | 82.5 |
| 216 | 69.1 | 79.9 | 57.5 | 65.7 | 80.9 | 49.2 |
| 78 | 66.9 | 71.3 | 62.1 | 67.3 | 74.0 | 60.0 |
| 69 | 67.1 | 68.0 | 66.1 | 69.7 | 67.9 | 71.7 |
| 217 | 65.9 | 75.6 | 55.4 | 68.1 | 78.6 | 56.7 |
| 82 | 67.2 | 78.9 | 54.6 | 68.1 | 77.1 | 58.3 |
| 68 | 66.7 | 77.9 | 54.6 | 67.7 | 74.8 | 60.0 |
| 207 | 65.2 | 63.0 | 67.5 | 68.9 | 57.3 | 81.7 |
| 89 | 66.0 | 81.2 | 49.6 | 64.5 | 80.2 | 47.5 |
| 218 | 65.7 | 73.6 | 57.1 | 61.4 | 71.8 | 50.0 |
| 135 | 67.9 | 84.2 | 50.4 | 64.1 | 80.2 | 46.7 |
| 198 | 62.3 | 74.6 | 48.9 | 61.8 | 77.1 | 45.0 |
| 219 | 60.4 | 60.1 | 60.7 | 62.2 | 58.8 | 65.8 |
| 160 | 63.0 | 65.0 | 60.7 | 60.2 | 54.2 | 66.7 |
| 169 | 59.0 | 60.4 | 57.5 | 66.5 | 63.4 | 70.0 |
| 271 | 60.9 | 59.1 | 62.9 | 65.3 | 62.6 | 68.3 |
| 11 | 64.0 | 65.0 | 62.9 | 61.0 | 62.6 | 59.2 |
| 220 | 61.7 | 63.0 | 60.4 | 63.3 | 59.5 | 67.5 |
| 76 | 63.0 | 63.4 | 62.5 | 64.9 | 63.4 | 66.7 |
| 268 | 58.8 | 65.0 | 52.1 | 58.6 | 63.4 | 53.3 |
| 127 | 62.6 | 61.1 | 64.3 | 66.1 | 60.3 | 72.5 |
| 272 | 60.0 | 64.4 | 55.4 | 64.9 | 65.6 | 64.2 |
| 196 | 62.8 | 68.3 | 56.8 | 60.2 | 58.8 | 61.7 |
| 85 | 61.7 | 65.3 | 57.9 | 67.3 | 67.9 | 66.7 |
| 128 | 62.6 | 80.2 | 43.6 | 61.4 | 80.9 | 40.0 |
| 211 | 64.5 | 64.0 | 65.0 | 63.7 | 61.1 | 66.7 |
| 266 | 60.0 | 62.4 | 57.5 | 64.9 | 62.6 | 67.5 |
| 273 | 59.9 | 64.4 | 55.0 | 65.3 | 67.2 | 63.3 |
| 221 | 62.4 | 65.7 | 58.9 | 66.9 | 71.0 | 62.5 |
| 274 | 61.6 | 66.0 | 56.8 | 62.5 | 61.1 | 64.2 |
| 99 | 59.5 | 53.8 | 65.7 | 66.1 | 61.1 | 71.7 |
| 4 | 62.1 | 77.6 | 45.4 | 59.0 | 65.6 | 51.7 |
| 275 | 57.6 | 60.7 | 54.3 | 63.3 | 63.4 | 63.3 |
| 222 | 59.7 | 62.0 | 57.1 | 59.8 | 54.2 | 65.8 |
| 109 | 60.9 | 71.0 | 50.0 | 57.8 | 64.9 | 50.0 |
| 264 | 58.0 | 62.0 | 53.6 | 67.7 | 70.2 | 65.0 |
| 267 | 58.0 | 73.9 | 40.7 | 59.0 | 77.1 | 39.2 |
| 223 | 62.8 | 64.0 | 61.4 | 64.1 | 66.4 | 61.7 |
| 224 | 56.6 | 61.1 | 51.8 | 58.6 | 55.7 | 61.7 |
| 103 | 59.2 | 63.7 | 54.3 | 61.8 | 65.6 | 57.5 |
| 225 | 59.9 | 63.4 | 56.1 | 63.3 | 65.6 | 60.8 |
| 226 | 59.2 | 61.7 | 56.4 | 61.0 | 66.4 | 55.0 |
| 227 | 55.7 | 56.8 | 54.6 | 54.2 | 48.9 | 60.0 |
| 105 | 59.3 | 68.0 | 50.0 | 59.0 | 68.7 | 48.3 |

TABLE 12

| SEQ ID NO. | Coefficient | Constant term |
|---|---|---|
| 4 | 0.922 | 12.664 |
| 11 | 1.105 | 14.004 |
| 17 | 0.960 | 7.747 |
| 68 | 1.375 | 12.584 |
| 69 | 0.985 | 9.816 |
| 76 | 0.965 | 6.289 |
| 78 | 1.556 | 12.359 |
| 82 | 1.331 | 8.889 |
| 85 | 0.947 | 9.520 |
| 89 | 1.112 | 6.837 |
| 99 | 0.380 | 3.958 |
| 103 | 0.509 | 3.812 |
| 105 | 0.585 | 5.043 |
| 109 | 0.897 | 7.611 |
| 127 | 0.695 | 4.323 |
| 128 | 1.011 | 5.816 |
| 135 | 0.904 | 5.237 |
| 145 | 1.582 | 13.004 |
| 160 | 0.891 | 6.528 |
| 164 | 0.601 | 4.237 |
| 169 | 0.977 | 7.088 |
| 196 | 1.000 | 7.867 |
| 198 | 0.884 | 5.915 |
| 207 | 0.644 | 3.437 |
| 211 | 0.882 | 6.012 |
| 216 | 1.037 | 7.417 |
| 217 | 1.063 | 7.712 |
| 218 | 0.904 | 6.645 |
| 219 | 0.688 | 3.697 |
| 220 | 0.807 | 4.862 |
| 221 | 0.652 | 3.708 |
| 222 | 0.646 | 3.514 |
| 223 | 0.586 | 3.132 |
| 224 | 0.826 | 4.941 |
| 225 | 0.562 | 3.235 |
| 226 | 0.614 | 3.688 |
| 227 | 0.697 | 4.274 |
| 264 | 0.463 | 3.401 |
| 266 | 0.636 | 4.190 |
| 267 | 0.670 | 4.758 |
| 268 | 1.011 | 7.943 |
| 271 | 0.631 | 3.810 |
| 272 | 0.482 | 2.852 |
| 273 | 0.448 | 2.494 |
| 274 | 0.661 | 3.646 |
| 275 | 0.544 | 3.116 |

TABLE 13

| | Training cohort | | | Validation cohort | | |
|---|---|---|---|---|---|---|
| | Accuracy | Sensitivity | Specificity | Accuracy | Sensitivity | |
| SEQ ID NO. | (%) | (%) | (%) | (%) | (%) | Specificity (%) |
| 164_4 | 71.0 | 69.6 | 72.5 | 73.3 | 67.9 | 79.2 |
| 17_11 | 73.1 | 81.8 | 63.6 | 74.1 | 79.4 | 68.3 |
| 17_82 | 78.9 | 91.4 | 65.4 | 80.5 | 90.1 | 70.0 |
| 82_68 | 76.2 | 85.5 | 66.1 | 76.1 | 79.4 | 72.5 |
| 69_82 | 75.1 | 81.2 | 68.6 | 79.7 | 81.7 | 77.5 |
| 82_76 | 77.0 | 80.9 | 72.9 | 80.1 | 78.6 | 81.7 |
| 164_78 | 75.8 | 76.9 | 74.6 | 78.5 | 77.9 | 79.2 |
| 164_82 | 80.1 | 82.5 | 77.5 | 83.3 | 81.7 | 85.0 |
| 82_85 | 71.9 | 78.5 | 64.6 | 79.7 | 83.2 | 75.8 |
| 164_89 | 80.1 | 82.8 | 77.1 | 82.5 | 84.0 | 80.8 |
| 145_99 | 67.2 | 69.3 | 65.0 | 73.7 | 79.4 | 67.5 |
| 217_103 | 69.3 | 75.6 | 62.5 | 72.9 | 80.9 | 64.2 |
| 164_105 | 68.6 | 63.4 | 74.3 | 74.5 | 64.9 | 85.0 |
| 69_109 | 68.4 | 71.3 | 65.4 | 71.3 | 69.5 | 73.3 |
| 82_127 | 75.0 | 79.2 | 70.4 | 79.3 | 80.2 | 78.3 |
| 164_128 | 78.2 | 82.2 | 73.9 | 77.7 | 77.9 | 77.5 |
| 17_135 | 75.5 | 86.1 | 63.9 | 77.3 | 87.0 | 66.7 |
| 145_164 | 75.3 | 77.2 | 73.2 | 78.5 | 77.1 | 80.0 |
| 82_160 | 81.6 | 86.5 | 76.4 | 82.5 | 83.2 | 81.7 |
| 164_82 | 80.1 | 82.5 | 77.5 | 83.3 | 81.7 | 85.0 |
| 82_169 | 74.6 | 81.2 | 67.5 | 80.5 | 81.7 | 79.2 |
| 82_196 | 78.9 | 87.8 | 69.3 | 83.3 | 85.5 | 80.8 |
| 164_198 | 75.6 | 76.9 | 74.3 | 77.7 | 77.9 | 77.5 |
| 82_207 | 77.7 | 80.2 | 75.0 | 78.5 | 74.8 | 82.5 |
| 82_211 | 76.5 | 80.5 | 72.1 | 76.5 | 76.3 | 76.7 |
| 164_216 | 76.3 | 78.9 | 73.6 | 77.7 | 80.9 | 74.2 |
| 164_217 | 75.3 | 79.2 | 71.1 | 78.5 | 77.9 | 79.2 |
| 164_218 | 77.2 | 78.9 | 75.4 | 76.9 | 77.1 | 76.7 |
| 82_219 | 76.7 | 81.5 | 71.4 | 76.9 | 75.6 | 78.3 |
| 82_220 | 79.4 | 83.5 | 75.0 | 82.5 | 84.0 | 80.8 |
| 82_221 | 75.1 | 81.8 | 67.9 | 80.1 | 84.7 | 75.0 |
| 145_222 | 69.8 | 75.9 | 63.2 | 73.3 | 74.8 | 71.7 |
| 82_223 | 69.0 | 76.6 | 60.7 | 75.3 | 80.9 | 69.2 |
| 78_224 | 68.8 | 71.3 | 66.1 | 74.1 | 79.4 | 68.3 |
| 82_225 | 69.6 | 77.9 | 60.7 | 72.5 | 80.2 | 64.2 |
| 217_226 | 67.9 | 73.6 | 61.8 | 71.7 | 80.9 | 61.7 |
| 82_227 | 73.1 | 80.2 | 65.4 | 74.1 | 74.8 | 73.3 |
| 82_264 | 70.8 | 78.2 | 62.9 | 74.1 | 79.4 | 68.3 |
| 145_266 | 67.9 | 69.6 | 66.1 | 73.3 | 77.9 | 68.3 |
| 145_267 | 69.0 | 74.6 | 62.9 | 73.7 | 83.2 | 63.3 |
| 160_268 | 80.4 | 77.2 | 83.9 | 80.9 | 77.1 | 85.0 |
| 82_271 | 72.4 | 76.6 | 67.9 | 75.3 | 76.3 | 74.2 |
| 82_272 | 67.9 | 74.6 | 60.7 | 71.7 | 71.0 | 72.5 |
| 164_273 | 68.1 | 64.0 | 72.5 | 73.3 | 64.9 | 82.5 |
| 145_274 | 68.8 | 72.6 | 64.6 | 73.3 | 77.9 | 68.3 |
| 217_275 | 68.6 | 75.2 | 61.4 | 73.7 | 80.2 | 66.7 |

### Example 4

### <Method for Evaluating Ovarian Tumor Discriminant Performance by Combination of a Plurality of Gene Markers>

In this Example, healthy subjects, benign bone and soft tissue tumor patients and benign breast disease patients, and patients having a cancer other than ovarian cancer (i.e., test subjects having no ovarian tumor) used in Examples 1, 2 and 3 were used as a negative sample group; and ovarian tumor patients were used as a positive sample group. The combinations of gene markers having high performance to discriminate an ovarian tumor were searched.

Specifically, miRNA expression levels in sera of 280 healthy subjects, 257 benign bone and soft tissue tumor patients and benign breast disease patients, 303 ovarian tumor patients, and 280 patients having a cancer other than ovarian cancer obtained in the Reference Example above were used as a training cohort; whereas miRNA expression levels in sera of 120 healthy subjects, 111 benign bone and soft tissue tumor patients and benign breast disease patients, 131 ovarian tumor patients and 120 patients having a cancer other than ovarian cancer were used as a validation cohort. In order to obtain a more highly reliable diagnostic marker(s), only a gene(s) having a gene expression level of 2⁶ or more in 50% or more samples in either one of a group of ovarian tumor patients or a group of test subjects having no ovarian tumor was selected.

Next, using combinations of 1 to 5 of these genes, discriminants were prepared by the Fisher's discriminant analysis and using the training cohort. Of the discriminants, those having the top to 30th ovarian tumor discriminant performance were searched for each of the above combinations of the genes. In the search, a modified greedy algorithm was used. Specifically, 30 genes singly showing satisfactory discriminant performance are used as pivots. Each of the genes used as pivots is used in combination with other genes and the Fisher's discriminant analysis was carried out. Discriminants (constituted of two genes) having the top to 30th discriminant performance are used as new pivots. This operation was repeated until the number of combined genes reached 5. In this manner, 150 discriminants for combined gene numbers having the top-level discriminant performance were obtained. As the genes constituting these discriminants, 73 genes represented by SEQ ID NOs: 2, 3, 4, 9, 11, 13, 15, 20, 33, 34, 38, 40, 44, 47, 56, 62, 68, 77, 78, 80, 82, 86, 89, 90, 91, 102, 104, 109, 117, 118, 135, 136, 145, 150, 157, 160, 161, 164, 169, 172, 196, 199, 211, 216, 217, 218, 220, 228 to 247, 250, 252, 256, 260, 267 and 268 were obtained (Table 14). Specifically, polynucleotides consisting of nucleotide sequences represented by the aforementioned SEQ ID NOs (SEQ ID NOs: 2, 3, 4, 9, 11, 13, 15, 20, 33, 34, 38, 40, 44, 47, 56, 62, 68, 77, 78, 80, 82, 86, 89, 90, 91, 102, 104, 109, 117, 118, 135, 136, 145, 150, 157, 160, 161, 164, 169, 172, 196, 199, 211, 216, 217, 218, 220, 228 to 247, 250, 252, 256, 260, 267 and 268) or complementary sequences thereof were found as target markers (specific polynucleotide group 1) which can specifically discriminate the presence or absence of an ovarian tumor.

The specific polynucleotide group 1 newly contains, in addition to the polynucleotides consisting of SEQ ID NOs: 2, 3, 4, 9, 11, 13, 15, 20, 33, 34, 38, 40, 44, 47, 56, 62, 68, 77, 78, 80, 82, 86, 89, 90, 91, 102, 104, 109, 117, 118, 135, 136, 145, 150, 157, 160, 161, 164, 169, 172, 196, 199, 250, 252, 256, 260, 267 and 268 and obtained in Example 1; the polynucleotide consisting of SEQ ID NO: 211 and obtained in Example 2; and the polynucleotides consisting of SEQ ID NOs: 216 to 218 and 220 and obtained in Example 3, hsa-miR-6794-5p, hsa-miR-4687-3p, hsa-miR-6743-5p, hsa-miR-6771-5p, hsa-miR-3141, hsa-miR-3162-5p, hsa-miR-4271, hsa-miR-1227-5p, hsa-miR-4257, hsa-miR-4270, hsa-miR-4516, hsa-miR-4651, hsa-miR-4725-3p, hsa-miR-6125, hsa-miR-6732-5p, hsa-miR-6791-5p, hsa-miR-6819-5p, hsa-miR-6891-5p, hsa-miR-7108-5p, and hsa-miR-7109-5p genes and the relevant SEQ ID NOs: 228, 229, 230, 231, 232, 233, 234, 235, 236, 237, 238, 239, 240, 241, 242, 243, 244, 245, 246 and 247.

The polynucleotides consisting of the nucleotide sequences represented by SEQ ID NOs: 228, 229, 230, 231, 232, 233, 234, 235, 236, 237, 238, 239, 240, 241, 242, 243, 244, 245, 246 and 247, which were newly found in this Example, are all genes newly found as the markers for examining the presence or absence of an ovarian tumor.

Of the above polynucleotides, four polynucleotides consisting of the nucleotide sequences represented by, for example, SEQ ID NOs: 260, 256, 20 and 89, had sensitivities of 87.0%, 80.9%, 77.9% and 77.9%, respectively in the validation cohort (Table 15). Herein, the general sensitivity of the existing marker CA-125 is reported as 77.4% (Non Patent Literature 4). Accordingly, it was demonstrated that the polynucleotides consisting of these sequences singly discriminate an ovarian tumor with sensitivity beyond CA-125.

These genes, if they are used not only singly but also in combinations of, for example, 2, 3, 4, 5, 6, 7, 8, 9, 10 or more, provide excellent ovarian tumor discriminant performance. For example, if a discriminant was prepared using gene expression level of the nucleotide sequence represented by SEQ ID NO: 256, alone, the discrimination accuracy in the validation cohort was 79.5%; however, if a discriminant was prepared using 2 genes (SEQ ID NOs: 256 and 196) in combination, the discrimination accuracy in the validation cohort was 84.4%, if a discriminant was prepared using 3 genes (SEQ ID NOs: 256, 196 and 260), the discrimination accuracy in the validation cohort was 87.6%, if a discriminant was prepared using 4 genes (SEQ ID NOs: 256, 196, 260 and 268), the discrimination accuracy in the validation cohort was 90.2%, and if a discriminant was prepared using 5 genes (SEQ ID NOs: 256, 196, 260, 268 and 252), the discrimination accuracy in the validation cohort was 92.3%.

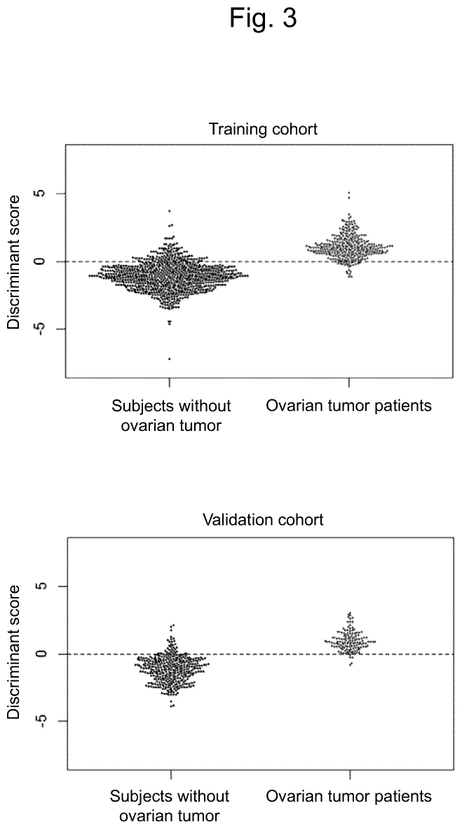

As to the discriminant prepared using measurement values of nucleotide sequences represented by SEQ ID NOs: 256, 196, 260, 268 and 252 in combination, discriminant scores of 303 ovarian tumor patients and 817 test subjects having no ovarian tumor in the training cohort are significantly separated, as shown in the upper panel of FIG. 3. The same results were able to be reproduced also in the validation cohort (FIG. 3, lower panel).

Of the 150 discriminants having the top-level discriminant performance and obtained above, the number of discriminants exhibiting a discrimination accuracy of 80% or more both in the training cohort and verification cohort was 94. Of them, 93 discriminants contained, as an explanatory variance, a target nucleic acid, which is any of polynucleotides consisting of nucleotide sequences represented by SEQ ID NOs: 228, 9, 196, 229, 145 or 164 or a complementary sequence thereof. In other words, of the combinations of a plurality of polynucleotides selected from the specific polynucleotide group 1, a combination containing at least one polynucleotide selected from the group (specific polynucleotide group 2) consisting of a polynucleotide consisting of the nucleotide sequence represented by SEQ ID NO: 228, 9, 196, 229, 145 or 164 or a complementary sequence thereof contained in the specific polynucleotide group 1 was able to specifically discriminate the presence or absence of an ovarian tumor with high accuracy.

Specifically, when measurement was carried out using a polynucleotide consisting of the nucleotide sequence represented by SEQ ID NO: 228 or a complementary sequence thereof as a target nucleic acid, discrimination accuracy is shown in Table 16-1. The measurement using a combination of 1, 2, 3, 4 or 5 genes comprising the polynucleotide consisting of the nucleotide sequence represented by SEQ ID NO: 228 or a complementary sequence thereof exhibited the highest accuracy of 58.9%, 81.7%, 85.5%, 89.8% and 91.3%, respectively, in the validation cohort.

Further, when measurement was carried out using a polynucleotide consisting of the nucleotide sequence represented by SEQ ID NO: 9 or a complementary sequence thereof as a target nucleic acid, discrimination accuracy is shown in Table 16-2. The measurement using a combination of 1, 2, 3, 4 or 5 genes comprising the polynucleotide consisting of the nucleotide sequence represented by SEQ ID NO: 9 or a complementary sequence thereof exhibited the highest accuracy of 66.8%, 79.0%, 85.7%, 89.8% and 91.1%, respectively, in the validation cohort.

Further, when measurement was carried out using a polynucleotide consisting of the nucleotide sequence represented by SEQ ID NO: 196 or a complementary sequence thereof as a target nucleic acid, discrimination accuracy is shown in Table 16-3. The measurement using a combination of 1, 2, 3, 4 or 5 genes comprising the polynucleotide consisting of the nucleotide sequence represented by SEQ ID NO: 196 or a complementary sequence thereof exhibited the highest accuracy of 58.9%, 84.4%, 87.6%, 90.2% and 92.3%, respectively, in the validation cohort.

Further, when measurement was carried out using a polynucleotide consisting of the nucleotide sequence represented by SEQ ID NO: 229 or a complementary sequence thereof as a target nucleic acid, discrimination accuracy is shown in Table 16-4. The measurement using a combination of 1, 2, 3, 4 or 5 genes comprising the polynucleotide consisting of the nucleotide sequence represented by SEQ ID NO: 229 or a complementary sequence thereof exhibited the highest accuracy of 74.9%, 81.5%, 85.9%, 89.4% and 91.3%, respectively, in the validation cohort.

Further, when measurement was carried out using a polynucleotide consisting of the nucleotide sequence represented by SEQ ID NO: 145 or a complementary sequence thereof as a target nucleic acid, discrimination accuracy is shown in Table 16-5. The measurement using a combination of 1, 2, 3, or 4 genes comprising the polynucleotide consisting of the nucleotide sequence represented by SEQ ID NO: 145 or a complementary sequence thereof exhibited the highest accuracy of 70.3%, 82.6%, 87.3% and 89.8%, respectively, in the validation cohort.

Further, when measurement was carried out using a polynucleotide consisting of the nucleotide sequence represented by SEQ ID NO: 164 or a complementary sequence thereof as a target nucleic acid, discrimination accuracy is shown in Table 16-6. The measurement using a combination of 1, 2 or 3 genes comprising the polynucleotide consisting of the nucleotide sequence represented by SEQ ID NO: 164 or a complementary sequence thereof exhibited thee highest accuracy of 53.1%, 81.1% and 85.5%, respectively, in the validation cohort.

The specific polynucleotide groups 1 and 2 obtained in this Example can be deemed to be gene groups based on which an ovarian tumor patient can be specifically discriminated from any of healthy subjects, benign bone and soft tissue tumor patients and benign breast disease patients, and patients having a cancer other than ovarian cancer. It was demonstrated that high ovarian tumor discriminant performance can be obtained by using a plurality of polynucleotides in combination as a target nucleic acid rather than using a single polynucleotide or a few polynucleotides in combination. Herein, the combination of the plurality of polynucleotides is not limited to those mentioned above. Even if the polynucleotides are used in any combination, an ovarian tumor can be detected.

TABLE 14

| SEQ ID NO. | Name of gene | ID |
|---|---|---|
| 2 | hsa-miR-4783-3p | MIMAT0019947 |
| 3 | hsa-miR-1228-5p | MIMAT0005582 |
| 4 | hsa-miR-4532 | MIMAT0019071 |
| 9 | hsa-miR-8073 | MIMAT0031000 |
| 11 | hsa-miR-1233-5p | MIMAT0022943 |
| 13 | hsa-miR-5195-3p | MIMAT0021127 |
| 15 | hsa-miR-4649-5p | MIMAT0019711 |
| 20 | hsa-miR-5100 | MIMAT0022259 |
| 33 | hsa-miR-6805-5p | MIMAT0027510 |
| 34 | hsa-miR-4758-5p | MIMAT0019903 |
| 38 | hsa-miR-6781-5p | MIMAT0027462 |
| 40 | hsa-miR-1273g-3p | MIMAT0022742 |
| 44 | hsa-miR-1249-3p | MIMAT0005901 |
| 47 | hsa-miR-1238-5p | MIMAT0022947 |
| 56 | hsa-miR-6775-5p | MIMAT0027450 |
| 62 | hsa-miR-6515-3p | MIMAT0025487 |
| 68 | hsa-miR-6787-5p | MIMAT0027474 |
| 77 | hsa-miR-6757-5p | MIMAT0027414 |
| 78 | hsa-miR-1275 | MIMAT0005929 |
| 80 | hsa-miR-6826-5p | MIMAT0027552 |
| 82 | hsa-miR-3679-3p | MIMAT0018105 |
| 86 | hsa-miR-4447 | MIMAT0018966 |
| 89 | hsa-miR-6766-3p | MIMAT0027433 |
| 90 | hsa-miR-197-5p | MIMAT0022691 |
| 91 | hsa-miR-6887-5p | MIMAT0027674 |
| 102 | hsa-miR-4734 | MIMAT0019859 |
| 104 | hsa-miR-638 | MIMAT0003308 |
| 109 | hsa-miR-8063 | MIMAT0030990 |
| 117 | hsa-miR-1908-5p | MIMAT0007881 |
| 118 | hsa-miR-6726-5p | MIMAT0027353 |
| 135 | hsa-miR-6798-3p | MIMAT0027497 |
| 136 | hsa-miR-665 | MIMAT0004952 |
| 145 | hsa-miR-7150 | MIMAT0028211 |
| 150 | hsa-miR-1229-5p | MIMAT0022942 |
| 157 | hsa-miR-6762-5p | MIMAT0027424 |
| 160 | hsa-miR-6746-5p | MIMAT0027392 |
| 161 | hsa-miR-6880-5p | MIMAT0027660 |
| 164 | hsa-miR-6717-5p | MIMAT0025846 |
| 169 | hsa-miR-6769a-5p | MIMAT0027438 |
| 172 | hsa-miR-6741-5p | MIMAT0027383 |
| 196 | hsa-miR-3131 | MIMAT0014996 |
| 199 | hsa-miR-504-3p | MIMAT0026612 |
| 211 | hsa-miR-6763-5p | MIMAT0027426 |
| 216 | hsa-miR-6870-5p | MIMAT0027640 |
| 217 | hsa-miR-6124 | MIMAT0024597 |
| 218 | hsa-miR-1249-5p | MIMAT0032029 |
| 220 | hsa-miR-1254 | MIMAT0005905 |
| 228 | hsa-miR-6794-5p | MIMAT0027488 |
| 229 | hsa-miR-4687-3p | MIMAT0019775 |
| 230 | hsa-miR-6743-5p | MIMAT0027387 |
| 231 | hsa-miR-6771-5p | MIMAT0027442 |
| 232 | hsa-miR-3141 | MIMAT0015010 |
| 233 | hsa-miR-3162-5p | MIMAT0015036 |
| 234 | hsa-miR-4271 | MIMAT0016901 |
| 235 | hsa-miR-1227-5p | MIMAT0022941 |
| 236 | hsa-miR-4257 | MIMAT0016878 |
| 237 | hsa-miR-4270 | MIMAT0016900 |
| 238 | hsa-miR-4516 | MIMAT0019053 |
| 239 | hsa-miR-4651 | MIMAT0019715 |
| 240 | hsa-miR-4725-3p | MIMAT0019844 |
| 241 | hsa-miR-6125 | MIMAT0024598 |
| 242 | hsa-miR-6732-5p | MIMAT0027365 |
| 243 | hsa-miR-6791-5p | MIMAT0027482 |
| 244 | hsa-miR-6819-5p | MIMAT0027538 |
| 245 | hsa-miR-6891-5p | MIMAT0027682 |
| 246 | hsa-miR-7108-5p | MIMAT0028113 |
| 247 | hsa-miR-7109-5p | MIMAT0028115 |
| 250 | hsa-miR-328-5p | MIMAT0026486 |
| 252 | hsa-miR-128-2-5p | MIMAT0031095 |
| 256 | hsa-miR-296-5p | MIMAT0000690 |
| 260 | hsa-miR-1290 | MIMAT0005880 |
| 267 | hsa-miR-422a | MIMAT0001339 |
| 268 | hsa-miR-642a-3p | MIMAT0020924 |

TABLE 15

| | Training cohort | | | Validation cohort | | |
|---|---|---|---|---|---|---|
| | Accuracy | Sensitivity | Specificity | Accuracy | Sensitivity | |
| SEQ ID NO. | (%) | (%) | (%) | (%) | (%) | Specificity (%) |
| 2 | 61.0 | 81.8 | 53.2 | 61.2 | 74.8 | 56.1 |
| 3 | 50.4 | 62.0 | 46.0 | 55.2 | 71.0 | 49.3 |
| 4 | 69.2 | 82.5 | 64.3 | 64.7 | 72.5 | 61.8 |
| 9 | 66.0 | 73.9 | 63.0 | 66.8 | 73.3 | 64.4 |
| 11 | 68.8 | 72.6 | 67.3 | 66.8 | 66.4 | 67.0 |
| 13 | 65.0 | 75.2 | 61.2 | 66.0 | 64.9 | 66.4 |
| 15 | 66.2 | 74.9 | 63.0 | 66.0 | 62.6 | 67.2 |
| 20 | 73.5 | 82.2 | 70.3 | 74.1 | 77.9 | 72.6 |
| 33 | 61.3 | 68.6 | 58.6 | 64.1 | 70.2 | 61.8 |
| 34 | 63.5 | 67.0 | 62.2 | 61.4 | 61.8 | 61.3 |
| 38 | 63.5 | 66.3 | 62.4 | 65.1 | 65.6 | 65.0 |
| 40 | 67.0 | 69.0 | 66.2 | 70.3 | 69.5 | 70.7 |
| 44 | 68.1 | 76.9 | 64.9 | 68.9 | 72.5 | 67.5 |
| 47 | 66.4 | 69.6 | 65.2 | 66.6 | 74.0 | 63.8 |
| 56 | 63.4 | 69.3 | 61.2 | 66.2 | 59.5 | 68.7 |
| 62 | 69.6 | 75.2 | 67.6 | 72.8 | 74.8 | 72.1 |
| 68 | 48.7 | 53.5 | 46.9 | 47.3 | 58.8 | 43.0 |
| 77 | 56.8 | 59.4 | 55.8 | 62.7 | 64.9 | 61.8 |
| 78 | 72.7 | 71.3 | 73.2 | 77.8 | 74.0 | 79.2 |
| 80 | 61.9 | 67.7 | 59.7 | 65.4 | 65.6 | 65.2 |
| 82 | 68.8 | 77.6 | 65.5 | 73.0 | 76.3 | 71.8 |
| 86 | 62.3 | 71.6 | 58.9 | 66.6 | 69.5 | 65.5 |
| 89 | 67.9 | 77.6 | 64.3 | 72.4 | 77.9 | 70.4 |
| 90 | 60.0 | 55.1 | 61.8 | 65.6 | 61.8 | 67.0 |
| 91 | 57.4 | 63.7 | 55.1 | 62.4 | 61.1 | 63.0 |
| 102 | 61.2 | 61.4 | 61.2 | 59.3 | 47.3 | 63.8 |
| 104 | 64.6 | 64.4 | 64.7 | 67.2 | 61.8 | 69.2 |
| 109 | 63.3 | 68.6 | 61.3 | 63.9 | 60.3 | 65.2 |
| 117 | 62.6 | 58.7 | 64.0 | 63.9 | 55.0 | 67.2 |
| 118 | 64.1 | 61.7 | 65.0 | 64.9 | 60.3 | 66.7 |
| 135 | 67.6 | 79.9 | 63.0 | 71.8 | 74.8 | 70.7 |
| 136 | 70.8 | 74.6 | 69.4 | 74.9 | 70.2 | 76.6 |
| 145 | 68.8 | 64.4 | 70.5 | 70.3 | 67.2 | 71.5 |
| 150 | 63.5 | 59.4 | 65.0 | 70.3 | 63.4 | 72.9 |
| 157 | 58.8 | 62.7 | 57.4 | 61.0 | 62.6 | 60.4 |
| 160 | 48.8 | 48.5 | 49.0 | 41.3 | 43.5 | 40.5 |
| 161 | 56.2 | 49.2 | 58.8 | 55.2 | 48.9 | 57.5 |
| 164 | 56.1 | 49.2 | 58.6 | 53.1 | 48.9 | 54.7 |
| 169 | 49.2 | 44.2 | 51.0 | 46.9 | 48.1 | 46.4 |
| 172 | 55.7 | 57.1 | 55.2 | 58.5 | 56.5 | 59.3 |
| 196 | 53.9 | 57.4 | 52.6 | 58.9 | 58.0 | 59.3 |
| 199 | 57.9 | 63.4 | 55.8 | 59.1 | 61.8 | 58.1 |
| 211 | 69.1 | 55.1 | 74.3 | 68.0 | 57.3 | 72.1 |
| 216 | 54.8 | 61.4 | 52.4 | 53.9 | 62.6 | 50.7 |
| 217 | 61.7 | 64.4 | 60.7 | 60.0 | 64.1 | 58.4 |
| 218 | 56.1 | 58.7 | 55.1 | 56.0 | 60.3 | 54.4 |
| 220 | 57.4 | 49.8 | 60.2 | 52.3 | 42.0 | 56.1 |
| 228 | 62.9 | 66.7 | 61.6 | 58.9 | 61.1 | 58.1 |
| 229 | 69.2 | 56.4 | 73.9 | 74.9 | 61.1 | 80.1 |
| 230 | 59.8 | 63.7 | 58.4 | 56.4 | 57.3 | 56.1 |
| 231 | 61.5 | 62.7 | 61.1 | 62.0 | 58.8 | 63.2 |
| 232 | 57.5 | 58.4 | 57.2 | 61.8 | 61.1 | 62.1 |
| 233 | 52.4 | 47.5 | 54.2 | 52.1 | 46.6 | 54.1 |
| 234 | 64.9 | 60.7 | 66.5 | 67.2 | 60.3 | 69.8 |
| 235 | 58.4 | 54.1 | 60.0 | 57.7 | 52.7 | 59.5 |
| 236 | 48.7 | 47.5 | 49.1 | 45.2 | 47.3 | 44.4 |
| 237 | 50.5 | 47.5 | 51.7 | 55.0 | 48.1 | 57.5 |
| 238 | 64.4 | 62.7 | 65.0 | 62.7 | 55.7 | 65.2 |
| 239 | 63.0 | 57.1 | 65.2 | 62.9 | 55.0 | 65.8 |
| 240 | 50.9 | 53.8 | 49.8 | 46.5 | 47.3 | 46.2 |
| 241 | 66.0 | 61.4 | 67.7 | 63.7 | 55.0 | 67.0 |
| 242 | 69.9 | 65.7 | 71.5 | 66.2 | 58.0 | 69.2 |
| 243 | 65.1 | 62.7 | 66.0 | 62.4 | 51.9 | 66.4 |
| 244 | 55.3 | 51.5 | 56.7 | 56.6 | 55.7 | 57.0 |
| 245 | 51.6 | 60.4 | 48.3 | 53.5 | 63.4 | 49.9 |
| 246 | 57.1 | 55.8 | 57.6 | 57.5 | 56.5 | 57.8 |
| 247 | 59.0 | 56.8 | 59.9 | 59.5 | 55.0 | 61.3 |
| 250 | 63.6 | 71.9 | 60.5 | 67.8 | 69.5 | 67.2 |
| 252 | 67.5 | 73.6 | 65.2 | 68.5 | 64.9 | 69.8 |
| 256 | 72.5 | 80.5 | 69.5 | 79.5 | 80.9 | 78.9 |
| 260 | 72.5 | 88.4 | 66.6 | 73.4 | 87.0 | 68.4 |
| 267 | 57.3 | 65.0 | 54.5 | 61.8 | 64.1 | 61.0 |
| 268 | 65.4 | 57.1 | 68.4 | 66.4 | 58.8 | 69.2 |

TABLE 16-1

| | Combined | Validation cohort | | |
|---|---|---|---|---|
| | gene | Accuracy | Sensitivity | Specificity |
| SEQ ID NO. | number | (%) | (%) | (%) |
| 228 | 1 | 58.9 | 61.1 | 58.1 |
| 256_228 | 2 | 81.7 | 78.6 | 82.9 |
| 47_228 | 2 | 80.9 | 83.2 | 80.1 |
| 228_9_229 | 3 | 85.7 | 86.3 | 85.5 |
| 228_9_78 | 3 | 83.6 | 87.8 | 82.1 |
| 228_9_82 | 3 | 83.0 | 88.5 | 80.9 |
| 228_9_268 | 3 | 83.8 | 87.0 | 82.6 |
| 229_260_228 | 3 | 85.5 | 87.0 | 84.9 |
| 228_9_247 | 3 | 81.3 | 86.3 | 79.5 |
| 228_9_62 | 3 | 84.9 | 89.3 | 83.2 |
| 228_9_217 | 3 | 81.5 | 86.3 | 79.8 |
| 256_228_9 | 3 | 84.4 | 88.5 | 82.9 |
| 256_228_260 | 3 | 83.8 | 90.1 | 81.5 |
| 228_9_234 | 3 | 80.9 | 82.4 | 80.3 |
| 229_20_228 | 3 | 85.3 | 86.3 | 84.9 |
| 47_228_82 | 3 | 83.0 | 87.0 | 81.5 |
| 145_260_228 | 3 | 85.5 | 91.6 | 83.2 |
| 228_9_145 | 3 | 82.6 | 90.1 | 79.8 |
| 228_9_268_160 | 4 | 88.0 | 89.3 | 87.5 |
| 228_9_82_268 | 4 | 85.5 | 88.5 | 84.3 |
| 228_9_268_196 | 4 | 86.7 | 87.8 | 86.3 |
| 145_260_228_250 | 4 | 89.8 | 95.4 | 87.7 |
| 145_260_228_62 | 4 | 88.0 | 93.9 | 85.8 |
| 228_9_268_230 | 4 | 86.1 | 89.3 | 84.9 |
| 229_260_228_2 | 4 | 87.1 | 87.0 | 87.2 |
| 228_9_268_62 | 4 | 86.5 | 90.1 | 85.2 |
| 229_260_228_172 | 4 | 87.1 | 87.0 | 87.2 |
| 229_260_228_4 | 4 | 86.9 | 89.3 | 86.0 |
| 229_260_228_34 | 4 | 85.9 | 84.7 | 86.3 |
| 229_260_228_56 | 4 | 88.4 | 90.8 | 87.5 |
| 229_20_228_4 | 4 | 86.9 | 87.8 | 86.6 |
| 229_20_228_102 | 4 | 85.5 | 81.7 | 86.9 |
| 145_260_228_89 | 4 | 89.6 | 95.4 | 87.5 |
| 228_9_229_82 | 4 | 87.8 | 88.5 | 87.5 |
| 228_9_78_62 | 4 | 86.7 | 90.1 | 85.5 |
| 229_260_228_157 | 4 | 87.1 | 88.5 | 86.6 |
| 229_260_228_231 | 4 | 89.4 | 90.8 | 88.9 |
| 256_228_260_232 | 4 | 87.8 | 91.6 | 86.3 |
| 228_9_229_117 | 4 | 85.3 | 82.4 | 86.3 |
| 228_9_229_62 | 4 | 86.9 | 86.3 | 87.2 |
| 228_9_78_172 | 4 | 84.9 | 87.8 | 83.8 |
| 228_9_78_3 | 4 | 84.0 | 83.2 | 84.3 |
| 228_9_82_268_196 | 5 | 89.0 | 91.6 | 88.0 |
| 229_20_228_4_172 | 5 | 87.3 | 86.3 | 87.7 |
| 228_9_82_268_233 | 5 | 87.8 | 90.8 | 86.6 |
| 228_9_268_196_89 | 5 | 88.0 | 90.8 | 86.9 |
| 228_9_268_160_82 | 5 | 89.8 | 91.6 | 89.2 |
| 228_9_268_196_256 | 5 | 91.1 | 95.4 | 89.5 |
| 228_9_78_62_238 | 5 | 87.1 | 90.1 | 86.0 |
| 228_9_268_196_172 | 5 | 87.1 | 88.5 | 86.6 |
| 256_228_260_232_196 | 5 | 90.0 | 94.7 | 88.3 |
| 228_9_268_160_230 | 5 | 88.6 | 90.1 | 88.0 |
| 256_196_260_268_228 | 5 | 90.9 | 95.4 | 89.2 |
| 228_9_268_196_62 | 5 | 89.8 | 90.8 | 89.5 |
| 228_9_268_196_244 | 5 | 85.7 | 87.8 | 84.9 |
| 228_9_268_196_135 | 5 | 87.8 | 89.3 | 87.2 |
| 229_260_228_2_217 | 5 | 88.6 | 92.4 | 87.2 |
| 228_9_268_62_233 | 5 | 87.6 | 90.8 | 86.3 |
| 229_260_228_172_230 | 5 | 88.8 | 90.1 | 88.3 |
| 229_260_228_56_33 | 5 | 90.2 | 91.6 | 89.7 |
| 229_260_228_56_231 | 5 | 91.3 | 92.4 | 90.9 |
| 229_260_228_231_91 | 5 | 90.2 | 93.9 | 88.9 |
| 228_9_268_160_245 | 5 | 88.6 | 89.3 | 88.3 |
| 228_9_82_268_237 | 5 | 86.1 | 90.1 | 84.6 |
| 228_9_82_268_236 | 5 | 88.2 | 90.1 | 87.5 |

TABLE 16-2

| | Combined | Validation cohort | | |
|---|---|---|---|---|
| | gene | Accuracy | Sensitivity | Specificity |
| SEQ ID NO. | number | (%) | (%) | (%) |
| 9 | 1 | 66.8 | 73.3 | 64.4 |
| 9_164 | 2 | 79.0 | 88.5 | 75.5 |
| 228_9_229 | 3 | 85.7 | 86.3 | 85.5 |
| 228_9_78 | 3 | 83.6 | 87.8 | 82.1 |
| 228_9_82 | 3 | 83.0 | 88.5 | 80.9 |
| 228_9_268 | 3 | 83.8 | 87.0 | 82.6 |
| 228_9_247 | 3 | 81.3 | 86.3 | 79.5 |
| 228_9_62 | 3 | 84.9 | 89.3 | 83.2 |
| 228_9_217 | 3 | 81.5 | 86.3 | 79.8 |
| 256_228_9 | 3 | 84.4 | 88.5 | 82.9 |
| 228_9_234 | 3 | 80.9 | 82.4 | 80.3 |
| 228_9_145 | 3 | 82.6 | 90.1 | 79.8 |
| 256_196_9 | 3 | 84.4 | 89.3 | 82.6 |
| 228_9_268_160 | 4 | 88.0 | 89.3 | 87.5 |
| 256_196_9_68 | 4 | 85.9 | 88.5 | 84.9 |
| 228_9_82_268 | 4 | 85.5 | 88.5 | 84.3 |
| 228_9_268_196 | 4 | 86.7 | 87.8 | 86.3 |
| 228_9_268_230 | 4 | 86.1 | 89.3 | 84.9 |
| 228_9_268_62 | 4 | 86.5 | 90.1 | 85.2 |
| 228_9_229_82 | 4 | 87.8 | 88.5 | 87.5 |
| 228_9_78_62 | 4 | 86.7 | 90.1 | 85.5 |
| 228_9_229_117 | 4 | 85.3 | 82.4 | 86.3 |
| 228_9_229_62 | 4 | 86.9 | 86.3 | 87.2 |
| 228_9_78_172 | 4 | 84.9 | 87.8 | 83.8 |
| 228_9_78_3 | 4 | 84.0 | 83.2 | 84.3 |
| 228_9_82_268_196 | 4 | 89.0 | 91.6 | 88.0 |
| 228_9_82_268_233 | 4 | 87.8 | 90.8 | 86.6 |
| 228_9_268_196_89 | 4 | 88.0 | 90.8 | 86.9 |
| 228_9_268_160_82 | 4 | 89.8 | 91.6 | 89.2 |
| 256_196_9_68_86 | 5 | 87.8 | 88.5 | 87.5 |
| 228_9_268_196_256 | 5 | 91.1 | 95.4 | 89.5 |
| 228_9_78_62_238 | 5 | 87.1 | 90.1 | 86.0 |
| 228_9_268_196_172 | 5 | 87.1 | 88.5 | 86.6 |
| 228_9_268_160_230 | 5 | 88.6 | 90.1 | 88.0 |
| 228_9_268_196_62 | 5 | 89.8 | 90.8 | 89.5 |
| 228_9_268_196_244 | 5 | 85.7 | 87.8 | 84.9 |
| 228_9_268_196_135 | 5 | 87.8 | 89.3 | 87.2 |
| 228_9_268_62_233 | 5 | 87.6 | 90.8 | 86.3 |
| 228_9_268_160_245 | 5 | 88.6 | 89.3 | 88.3 |
| 256_196_9_68_268 | 5 | 89.4 | 90.8 | 88.9 |
| 228_9_82_268_237 | 5 | 86.1 | 90.1 | 84.6 |
| 228_9_82_268_236 | 5 | 88.2 | 90.1 | 87.5 |

TABLE 16-3

| | Combined | Validation cohort | | |
|---|---|---|---|---|
| | gene | Accuracy | Sensitivity | Specificity |
| SEQ ID NO. | number | (%) | (%) | (%) |
| 196 | 1 | 58.9 | 58.0 | 59.3 |
| 256_196 | 2 | 84.4 | 84.7 | 84.3 |
| 256_196_260 | 3 | 87.6 | 93.9 | 85.2 |
| 256_196_172 | 3 | 85.5 | 84.7 | 85.8 |
| 256_196_47 | 3 | 86.7 | 93.1 | 84.3 |
| 256_196_11 | 3 | 85.1 | 88.5 | 83.8 |
| 256_196_136 | 3 | 85.9 | 85.5 | 86.0 |
| 256_196_80 | 3 | 86.9 | 90.1 | 85.8 |
| 256_196_9 | 3 | 84.4 | 89.3 | 82.6 |
| 256_196_260_268 | 4 | 90.2 | 93.9 | 88.9 |
| 256_196_9_68 | 4 | 85.9 | 88.5 | 84.9 |
| 256_196_260_216 | 4 | 90.2 | 96.9 | 87.7 |
| 256_196_260_11 | 4 | 89.0 | 93.1 | 87.5 |
| 228_9_268_196 | 4 | 86.7 | 87.8 | 86.3 |
| 256_196_260_217 | 4 | 89.6 | 93.9 | 88.0 |
| 228_9_82_268_196 | 5 | 89.0 | 91.6 | 88.0 |
| 228_9_268_196_89 | 5 | 88.0 | 90.8 | 86.9 |
| 256_196_9_68_86 | 5 | 87.8 | 88.5 | 87.5 |
| 228_9_268_196_256 | 5 | 91.1 | 95.4 | 89.5 |
| 256_196_260_268_252 | 5 | 92.3 | 96.2 | 90.9 |
| 228_9_268_196_172 | 5 | 87.1 | 88.5 | 86.6 |
| 256_228_260_232_196 | 5 | 90.0 | 94.7 | 88.3 |
| 256_196_260_268_228 | 5 | 90.9 | 95.4 | 89.2 |
| 256_196_260_268_218 | 5 | 90.9 | 94.7 | 89.5 |
| 256_196_260_268_267 | 5 | 90.9 | 96.2 | 88.9 |
| 228_9_268_196_62 | 5 | 89.8 | 90.8 | 89.5 |
| 228_9_268_196_244 | 5 | 85.7 | 87.8 | 84.9 |
| 228_9_268_196_135 | 5 | 87.8 | 89.3 | 87.2 |
| 256_196_260_217_199 | 5 | 89.2 | 94.7 | 87.2 |
| 256_196_260_268_161 | 5 | 91.5 | 96.2 | 89.7 |
| 256_196_9_68_268 | 5 | 89.4 | 90.8 | 88.9 |

TABLE 16-4

| | Combined | Validation cohort | | |
|---|---|---|---|---|
| | gene | Accuracy | Sensitivity | Specificity |
| SEQ ID NO. | number | (%) | (%) | (%) |
| 229 | 1 | 74.9 | 61.1 | 80.1 |
| 229_256 | 2 | 81.5 | 74.8 | 84.0 |
| 228_9_229 | 3 | 85.7 | 86.3 | 85.5 |
| 229_260_228 | 3 | 85.5 | 87.0 | 84.9 |
| 229_260_230 | 3 | 85.9 | 88.5 | 84.9 |
| 229_260_169 | 3 | 83.0 | 84.7 | 82.3 |
| 229_260_160 | 3 | 83.0 | 78.6 | 84.6 |
| 229_20_228 | 3 | 85.3 | 86.3 | 84.9 |
| 229_260_228_2 | 4 | 87.1 | 87.0 | 87.2 |
| 229_260_169_2 | 4 | 89.2 | 90.1 | 88.9 |
| 229_260_228_172 | 4 | 87.1 | 87.0 | 87.2 |
| 229_260_228_4 | 4 | 86.9 | 89.3 | 86.0 |
| 229_260_228_34 | 4 | 85.9 | 84.7 | 86.3 |
| 229_260_228_56 | 4 | 88.4 | 90.8 | 87.5 |
| 229_20_228_4 | 4 | 86.9 | 87.8 | 86.6 |
| 229_20_228_102 | 4 | 85.5 | 81.7 | 86.9 |
| 228_9_229_82 | 4 | 87.8 | 88.5 | 87.5 |
| 229_260_228_157 | 4 | 87.1 | 88.5 | 86.6 |
| 229_260_228_231 | 4 | 89.4 | 90.8 | 88.9 |
| 228_9_229_117 | 4 | 85.3 | 82.4 | 86.3 |
| 228_9_229_62 | 4 | 86.9 | 86.3 | 87.2 |
| 229_20_228_4_172 | 5 | 87.3 | 86.3 | 87.7 |
| 229_260_228_2_217 | 5 | 88.6 | 92.4 | 87.2 |
| 229_260_228_172_230 | 5 | 88.8 | 90.1 | 88.3 |
| 229_260_228_56_33 | 5 | 90.2 | 91.6 | 89.7 |
| 229_260_228_56_231 | 5 | 91.3 | 92.4 | 90.9 |
| 229_260_228_231_91 | 5 | 90.2 | 93.9 | 88.9 |

TABLE 16-5

| | Combined | Validation cohort | | |
|---|---|---|---|---|
| | gene | Accuracy | Sensitivity | Specificity |
| | number | (%) | (%) | (%) |
| 145 | 1 | 70.3 | 67.2 | 71.5 |
| 145_256 | 2 | 82.6 | 84.0 | 82.1 |
| 145_260_164 | 3 | 82.0 | 90.8 | 78.6 |
| 145_260_230 | 3 | 83.4 | 88.5 | 81.5 |
| 145_260_211 | 3 | 87.3 | 88.5 | 86.9 |
| 145_260_228 | 3 | 85.5 | 91.6 | 83.2 |
| 228_9_145 | 3 | 82.6 | 90.1 | 79.8 |
| 145_260_228_250 | 4 | 89.8 | 95.4 | 87.7 |
| 145_260_228_62 | 4 | 88.0 | 93.9 | 85.8 |
| 145_260_228_89 | 4 | 89.6 | 95.4 | 87.5 |

TABLE 16-6

| | Combined | Validation cohort | | |
|---|---|---|---|---|
| | gene | Accuracy | Sensitivity | Specificity |
| SEQ ID NO. | number | (%) | (%) | (%) |
| 164 | 1 | 53.1 | 48.9 | 54.7 |
| 164_256 | 2 | 81.1 | 80.9 | 81.2 |
| 11_164_260 | 3 | 85.3 | 86.3 | 84.9 |
| 11_164_20 | 3 | 85.5 | 84.7 | 85.8 |
| 145_260_164 | 3 | 82.0 | 90.8 | 78.6 |

Specifically, as shown in Tables 4 to 16 of Examples 1 to 4, it can be concluded that in all of the polynucleotides consisting of the nucleotide sequences represented by SEQ ID NOs: 1 to 275 or complementary sequences thereof as the target nucleic acid, a combination of 1, 2, 3, 4 or 5 exhibits discriminant performance beyond the existing ovarian tumor markers, and thus, such polynucleotides serve as excellent diagnostic markers.

As shown in the above Examples, the kit, device and method of the present invention can detect an ovarian tumor with higher sensitivity than the existing tumor markers and therefore permit early detection of an ovarian tumor. As a result, a treatment such as a chemotherapy, a radiotherapy, an immunotherapy, a molecular targeted therapy, or surgery with a high degree of probability for complete therapy can be early applied, significantly improving a survival rate.

### INDUSTRIAL APPLICABILITY

According to the present invention, an ovarian tumor can be effectively detected by a simple and inexpensive method. This enables early detection, diagnosis and treatment of an ovarian tumor. Also, the method of the present invention enables less-invasive detection of an ovarian tumor using patient's blood and therefore an ovarian tumor can be simply and quickly detected.

All publications, patents, and patent applications cited herein are incorporated herein by reference in their entirety.

The invention claimed is:

1. A method for detecting ovarian tumor, comprising:
1) measuring an expression level of miR-6765-3p in a sample from a subject; and evaluating in vitro whether or not the subject has ovarian tumor using the measured expression level;
2) measuring an expression level of a target gene in a sample from a subject using a kit or device comprising a nucleic acid capable of specifically binding to a polynucleotide of miR-6765-3p or to a complementary strand of the polynucleotide; and evaluating in vitro whether or not the subject has ovarian tumor using both of the measured expression levels and a control expression level from a healthy subject measured in the same way; or
3) measuring an expression level of a target gene in a sample from a subject using a kit or device comprising a nucleic acid capable of specifically binding a polynucleotide of miR-6765-3p or to a complementary strand of the polynucleotide; and assigning the expression level of the target gene in the sample from the subject to a discriminant, which is capable of discriminatorily determining the presence or absence of ovarian tumor and is prepared with gene expression levels in a sample(s) from a subject(s) known to have ovarian tumor and a sample(s) from a subject(s) without ovarian tumor as a training sample(s), and thereby evaluating in vitro the presence or absence of ovarian tumor.

2. The method according to claim 1, wherein the nucleic acid is a polynucleotide selected from the group consisting of the following polynucleotides (a) to (e):
(a) a polynucleotide consisting of a nucleotide sequence represented by SEQ ID NO: 81 or a nucleotide sequence derived from the nucleotide sequence by the replacement of u with t, a variant thereof, a derivative thereof, or a fragment thereof comprising 15 or more consecutive nucleotides;
(b) a polynucleotide comprising a nucleotide sequence represented by SEQ ID NO: 81 or a nucleotide sequence derived from the nucleotide sequence by the replacement of u with t;
(c) a polynucleotide consisting of a nucleotide sequence complementary to a nucleotide sequence represented by SEQ ID NO: 81 or a nucleotide sequence derived from the nucleotide sequence by the replacement of u with t, a variant thereof, a derivative thereof, or a fragment thereof comprising 15 or more consecutive nucleotides;
(d) a polynucleotide comprising a nucleotide sequence complementary to a nucleotide sequence represented by SEQ ID NO: 81 or a nucleotide sequence derived from the nucleotide sequence by the replacement of u with t; and
(e) a polynucleotide hybridizing under stringent conditions to any of the polynucleotides (a) to (d).

3. The method according to claim 1, wherein the kit or device further comprises a nucleic acid(s) capable of specifically binding to at least one polynucleotide selected from the following other ovarian tumor markers: miR-4675, miR-4783-3p, miR-1228-5p, miR-4532, miR-6802-5p, miR-6784-5p, miR-3940-5p, miR-1307-3p, miR-8073, miR-3184-5p, miR-6088, miR-5195-3p, miR-320b, miR-4649-5p, miR-6800-5p, miR-1343-3p, miR-4730, miR-6885-5p, miR-5100, miR-1203, miR-6756-5p, miR-373-5p, miR-1268a, miR-1260b, miR-4258, miR-4697-5p, miR-1469, miR-4515, miR-6861-5p, miR-6821-5p, miR-575, miR-6805-5p, miR-4758-5p, miR-3663-3p, miR-4530, miR-6798-5p, miR-6781-5p, miR-885-3p, miR-1273g-3p, miR-4787-3p, miR-4454, miR-4706, miR-1249-3p, miR-887-3p, miR-6786-5p, miR-1238-5p, miR-6749-5p, miR-6729-5p, miR-6825-5p, miR-663b, miR-6858-5p, miR-4690-5p, miR-6765-5p, miR-4710, miR-6775-5p, miR-371a-5p, miR-6816-5p, miR-296-3p, miR-7977, miR-8069, miR-6515-3p, miR-4687-5p, miR-1343-5p, miR-7110-5p, miR-4525, miR-3158-5p, miR-6787-5p, miR-614, miR-4689, miR-1268b, miR-1228-3p, miR-1185-1-3p, miR-940, miR-939-5p, miR-6757-5p, miR-1275, miR-5001-5p, miR-6826-5p, miR-3679-3p, miR-4718, miR-4286, miR-8059, miR-4447, miR-4448, miR-658, miR-6766-3p, miR-197-5p, miR-6887-5p, miR-6742-5p, miR-6729-3p, miR-5090, miR-7975, miR-4505, miR-6889-5p, miR-4708-3p, miR-6131, miR-1225-3p, miR-6132, miR-4734, miR-3194-3p, miR-638, miR-2467-3p, miR-4728-5p, miR-5572, miR-6789-5p, miR-8063, miR-4429, miR-6840-3p, miR-4476, miR-675-5p, miR-711, miR-6875-5p, miR-3160-5p, miR-1908-5p, miR-6726-5p, miR-1913, miR-8071, miR-3648, miR-4732-5p, miR-4787-5p, miR-3917, miR-619-5p, miR-1231, miR-342-5p, miR-4433a-5p, miR-6766-5p, miR-4707-5p, miR-7114-5p, miR-6872-3p, miR-6780b-5p, miR-7845-5p, miR-6798-3p, miR-665, miR-6848-5p, miR-5008-5p, miR-4294, miR-6511a-5p, miR-4435, miR-4747-3p, miR-6880-3p, miR-6869-5p, miR-7150, miR-1260a, miR-6877-5p, miR-6721-5p, miR-4656, miR-1229-5p, miR-4433a-3p, miR-4274, miR-4419b, miR-4674, miR-6893-5p, miR-6763-3p, miR-6762-5p, miR-6738-5p, miR-4513, miR-6746-5p, miR-6880-5p, miR-4736, miR-718, miR-6717-5p, miR-7847-3p, miR-760, miR-1199-5p, miR-6813-5p, miR-6769a-5p, miR-1193, miR-7108-3p, miR-6741-5p, miR-4298, miR-6796-3p, miR-4750-5p, miR-6785-5p, miR-1292-3p, miR-4749-3p, miR-6800-3p, miR-4722-5p, miR-4746-3p, miR-4450, miR-6795-5p, miR-365a-5p, miR-498, miR-6797-5p, miR-1470, miR-6851-5p, miR-1247-3p, miR-5196-5p, miR-208a-5p, miR-6842-5p, miR-150-3p, miR-4534, miR-3135b, miR-3131, miR-4792, miR-6510-5p, miR-504-3p, miR-3619-3p, miR-671-5p, miR-4667-5p, miR-4430, miR-3195, miR-3679-5p, miR-6076, miR-6515-5p, miR-6820-5p, miR-4634, miR-187-5p, miR-6763-5p, miR-1908-3p, miR-1181, miR-6782-5p, miR-5010-5p, miR-6870-5p, miR-6124, miR-1249-5p, miR-6511b-5p, miR-1254, miR-4727-3p, miR-4259, miR-4771, miR-3622a-5p, miR-4480, miR-4740-5p, miR-6777-5p, miR-6794-5p, miR-4687-3p, miR-6743-5p, miR-6771-5p, miR-3141, miR-3162-5p, miR-4271, miR-1227-5p, miR-4257, miR-4270, miR-4516, miR-4651, miR-4725-3p, miR-6125, miR-6732-5p, miR-6791-5p, miR-6819-5p, miR-6891-5p, miR-7108-5p, miR-7109-5p, miR-642b-3p, miR-642a-3p, miR-320a, miR-663a, miR-328-5p, miR-128-2-5p, miR-125a-3p, miR-191-5p, miR-92b-5p, miR-296-5p, miR-1246, miR-92a-2-5p, miR-128-1-5p, miR-1290, miR-211-3p, miR-744-5p, miR-135a-3p, miR-451a, miR-625-3p, miR-92a-3p, miR-422a, miR-483-5p, miR-652-5p, miR-24-3p, miR-23b-3p, miR-23a-3p, miR-92b-3p, and miR-22-3p, or to a complementary strand of the polynucleotide.

4. The method according to claim 3, wherein the nucleic acid(s) is a polynucleotide(s) selected from the group consisting of the following polynucleotides (a)′ to (e)′:
(a)′ a polynucleotide consisting of a nucleotide sequence represented by any of SEQ ID NOs: 1 to 10, 12 to 70, 72 to 80, 82 to 247, 251, and 268 or a nucleotide sequence derived from the nucleotide sequence by the replacement of u with t, a variant thereof, a derivative thereof, or a fragment thereof comprising 15 or more consecutive nucleotides;
(b)′ a polynucleotide comprising a nucleotide sequence represented by any of SEQ ID NOs: 1 to 10, 12 to 70, 72 to 80, 82 to 247, 251, and 268 or a nucleotide sequence derived from the nucleotide sequence by the replacement of u with t;
(c)′ a polynucleotide consisting of a nucleotide sequence complementary to a nucleotide sequence represented by any of SEQ ID NOs: 1 to 10, 12 to 70, 72 to 80, 82 to 247, 251, and 268 or a nucleotide sequence derived from the nucleotide sequence by the replacement of u with t, a variant thereof, a derivative thereof, or a fragment thereof comprising 15 or more consecutive nucleotides;
(d)′ a polynucleotide comprising a nucleotide sequence complementary to a nucleotide sequence represented by any of SEQ ID NOs: 1 to 10, 12 to 70, 72 to 80, 82 to 247, 251, and 268 or a nucleotide sequence derived from the nucleotide sequence by the replacement of u with t; and
(e)′ a polynucleotide hybridizing under stringent conditions to any of the polynucleotides (a)′ to (d)′; and/or
a polynucleotide(s) selected from the group consisting of the following polynucleotides (f) to (j):
(f) a polynucleotide consisting of a nucleotide sequence represented by any of SEQ ID NOs: 248 to 250, 252 to 267, and 269 to 275 or a nucleotide sequence derived from the nucleotide sequence by the replacement of u with t, a variant thereof, a derivative thereof, or a fragment thereof comprising 15 or more consecutive nucleotides;
(g) a polynucleotide comprising a nucleotide sequence represented by any of SEQ ID NOs: 248 to 250, 252 to 267, and 269 to 275 or a nucleotide sequence derived from the nucleotide sequence by the replacement of u with t;
(h) a polynucleotide consisting of a nucleotide sequence complementary to a nucleotide sequence represented by any of SEQ ID NOs: 248 to 250, 252 to 267, and 269 to 275 or a nucleotide sequence derived from the nucleotide sequence by the replacement of u with t, a variant thereof, a derivative thereof, or a fragment thereof comprising 15 or more consecutive nucleotides;
(i) a polynucleotide comprising a nucleotide sequence complementary to a nucleotide sequence represented by any of SEQ ID NOs: 248 to 250, 252 to 267, and 269 to 275 or a nucleotide sequence derived from the nucleotide sequence by the replacement of u with t; and
j) a polynucleotide hybridizing under stringent conditions to any of the polynucleotides (f) to (i).

5. The method according to claim 1, wherein the subject is a human.

6. The method according to claim 1, wherein the sample is blood, serum, or plasma.
